(19) United States
(12) Patent Application Publication
(10) Pub. No.: US 2025/0066350 A1
(43) Pub. Date: Feb. 27, 2025

(54) AROMATIC HETEROCYCLE-SUBSTITUTED COMPOUNDS, AND PREPARATION METHOD THEREFOR AND USE THEREOF

(71) Applicant: Innovstone Therapeutics Limited, Shanghai (CN)

(72) Inventors: Yunlong Song, Shanghai (CN); Wenqing Xu, Shanghai (CN); Xinyuan MIAO, Shanghai (CN); Kun ZHANG, Shanghai (CN); Dapei LI, Shanghai (CN); Jian CHEN, Shanghai (CN); Wei LI, Shanghai (CN); Kai LU, Shanghai (CN); Hongyan KOU, Shanghai (CN); Disha WANG, Shanghai (CN); Qiangqiang JIANG, Shanghai (CN)

(73) Assignee: Innovstone Therapeutics Limited, Shanghai (CN)

(21) Appl. No.: 18/720,535

(22) PCT Filed: Dec. 15, 2022
(86) PCT No.: PCT/CN2022/139194

§ 371 Date: Jun. 14, 2024

(30) Foreign Application Priority Data
Dec. 15, 2021 (CN) 202111577425.5
Jul. 20, 2022 (CN) 202210853001.5

(51) Int. Cl. C07D 471/04; A61K 31/5377; A61K 31/55; A61P 35/00; C07D 519/00

(52) U.S. Cl. CPC C07D 471/04; A61K 31/5377; A61K 31/55; A61P 35/00; C07D 519/00

(57) ABSTRACT

Disclosed are a class of compounds having a new structure as an ATR inhibitor, and a stereoisomer, an optical isomer, a pharmaceutical salt, a prodrug and a solvate thereof. An in-vitro enzyme inhibitory activity study shows that the compounds have a strong inhibitory effect on an ATR enzyme, and can be used as prospecting compounds for treating ATR-mediated diseases.

### TECHNICAL FIELD

The present invention relates to the technical field of medicines, and specifically relates to a class of aromatic heterocycle-substituted compounds, a preparation method therefor and the use thereof.

### BACKGROUND ART

ATR (Ataxia telangiectasia and Rad3-related protein) belongs to a class of protein kinases involved in genome stability and DNA damage repair, and is a member of the PIKK family. ATR can be activated by stalled replication forks or DNA single-strand breaks (SSBs). The activated ATR recruits repair proteins or factors to repair the damaged sites and delays the mitotic process (especially in the G2/M phase of mitosis), which not only stabilizes the replication forks, but also ensures the genome stability. Once activated, ATR activates three signaling pathways by regulating its downstream regulators (mainly including Chk1, WRN and FANCI) to block cell cycle progression, promote DNA repair and stabilize replication forks. Tumor cells harbor defects in some DNA repairs due to the presence of various mutations, therefore display a greater reliance on undamaged DNA repair pathways. The theory of synthetic lethality can be used to kill specific tumor cells while sparing healthy cells. Current cancer treatments, including chemotherapy and ionizing radiation, can induce DNA damage and replication fork stalling, so as to activate cell cycle checkpoints and lead to cell cycle arrest. This action mechanism is important in helping cancer cells survive the treatment. Broken double-stranded DNA or replication stress can rapidly activate ATR, and the corresponding ATR can activate a series of downstream targets such as Chk1 (ATR substrate), p53, and DNA topoisomerase 2-binding protein (TopBP1), leading to DNA repair and cell cycle arrest. The ATR gene is highly susceptible to activation during cancer chemotherapy because it is rarely mutated. Therefore, ATR inhibition can be used in combination with chemotherapeutic agents to synergistically enhance the effect.

Currently, some molecules disclosed in the prior art have entered the clinical phase, for example, Berzosertib disclosed in WO 2010071837 A1, Elimusertib disclosed in WO 2011154737 A1, and RP3500 disclosed in WO 2020087170 A1 are all in phase 1/11 clinical trials.

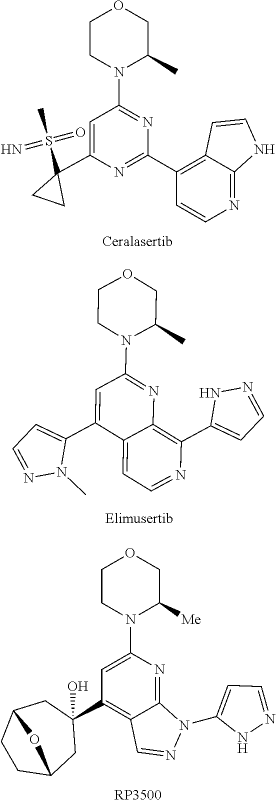 [SMILES: C[C@@H]1COCCN1c1cc(C2([S@](C)(=N)=O)CC2)nc(-c2ccnc3[nH]ccc23)n1 ; C[C@@H]1COCCN1c1cc(-c2ccnn2C)c2ccnc(-c3ccn[nH]3)c2n1 ; C[C@@H]1COCCN1c1cc([C@]2(O)C[C@H]3CC[C@@H](C2)O3)c2cnn(-c3ccn[nH]3)c2n1]

To date, there are no ATR inhibitors on the market. There is still a need to find ATR inhibitors with improved effect and safety.

### SUMMARY OF THE INVENTION

The objective of the present invention is to provide a compound with a novel structure as an ATR inhibitor, a method for preparing the compound and the use thereof in the treatment of an ATR-mediated disease.

A first aspect of the present invention provides a compound as shown in formula (A), and a stereoisomer, an optical isomer, a pharmaceutical salt, a prodrug and a solvate thereof,

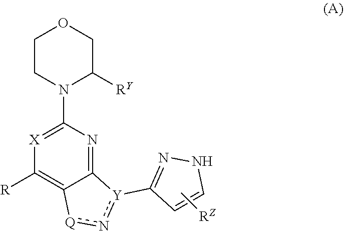

wherein,
one of the bond connecting Q and N and the bond connecting N and Y is a double bond; when the bond connecting Q and N is a double bond, the bond connecting N and Y is a single bond; when the bond connecting N and Y is a double bond, the bond connecting Q and N is a single bond;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{Y} is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl;
R is selected from one of C₆₋₁₂ aryl, 5- to 12-membered heteroaryl, C₂₋₆ alkynyl, C₃₋₁₂ carbocyclyl and 3- to 12-membered heterocyclyl, wherein the C₆₋₁₂ aryl, 5- to 12-membered heteroaryl, C₂₋₆ alkynyl, C₃₋₁₂ carbocyclyl and 3- to 12-membered heterocyclyl are optionally substituted with one or more of the following substituents: hydroxyl, sulfhydryl, amino, carboxyl, cyano, halogen, oxo, aminoacyl, C₁₋₆ alkyl, C₂₋₆ alkenyl, C₂₋₆ alkynyl, C₃₋₁₂ carbocyclyl, C₆₋₁₂ aryl, C₁₋₆ alkoxy, C₁₋₆ alkylthio, 3- to 12-membered heterocyclyl, 5- to 12-membered heteroaryl, —C₁₋₆ alkyl-NH₂, —NHC₁₋₆ alkyl, —NH-5- to 12-membered heteroaryl, —N(C₁₋₆ alkyl)₂, —NHCOC₁₋₆ alkyl, —NHCOC₃₋₆ carbocyclyl, —NHCOC₃₋₁₂ aryl, —NHCO-3- to 12-membered heteroaryl, —NHCO-3- to 12-membered heterocyclyl, —NHCONHC₁₋₆ alkyl, —NHCONHC₃₋₁₂ carbocyclyl, —NHCONH-3- to 12-membered heterocyclyl, —CONH C₁₋₆ alkyl, —CON(C₁₋₆ alkyl)₂, —C₁₋₆ alkyl-C₃₋₁₂ carbocyclyl, —C₁₋₆ alkyl-5- to 12-membered heteroaryl, —C₁₋₆ alkyl-3- to 12-membered heterocyclyl and —C₁₋₆ alkyl-C₆₋₁₂ aryl, wherein the C₁₋₆ alkyl, C₂₋₆ alkenyl, C₂₋₆ alkynyl, C₃₋₁₂ carbocyclyl, C₆₋₁₂ aryl, C₁₋₆ alkoxy, C₁₋₆ alkylthio, 3- to 12-membered heterocyclyl, 5- to 12-membered heteroaryl, —C₁₋₆ alkyl-NH₂, —NHC₁₋₆ alkyl, —NH-5- to 12-membered heteroaryl, —N(C₁₋₆ alkyl)₂, —NHCOC₁₋₆ alkyl, —NHCOC₃₋₆ carbocyclyl, —NHCOC₃₋₁₂ aryl, —NHCO-3- to 12-membered heteroaryl, —NHCO-3- to 12-membered heterocyclyl, —NHCONHC₁₋₆ alkyl, —NHCONHC₃₋₆ carbocyclyl, —CONHC₁₋₆ alkyl, —CON(C₁₋₆ alkyl)₂, —C₁₋₆ alkyl-C₃₋₁₂ carbocyclyl, —C₁₋₆ alkyl-5- to 12-membered heteroaryl, —C₁₋₆ alkyl-3- to 12-membered heterocyclyl and —C₁₋₆ alkyl-C₆₋₁₂ aryl are optionally substituted with one or more of the following substituents: hydroxyl, sulfhydryl, amino, carboxyl, cyano, halogen, oxo, amido, aminoacyl, —SO₂NH₂, C₁₋₆ alkyl optionally substituted with halogen or hydroxyl, C₂₋₆ alkenyl optionally substituted with halogen or hydroxyl, C₂₋₆ alkynyl optionally substituted with halogen or hydroxyl, C₁₋₆ alkoxy optionally substituted with halogen or hydroxyl, —C₁₋₆ alkyl-OH optionally substituted with halogen or hydroxyl, —C₁₋₆ alkyl-O—C₁₋₆ alkyl optionally substituted with halogen or hydroxyl, C₃₋₆ cycloalkyl optionally substituted with halogen or hydroxyl, C₆₋₁₂ aryl optionally substituted with halogen or hydroxyl, —CH₂—C₆₋₁₂ aryl optionally substituted with halogen or hydroxyl, 3- to 6-membered heterocyclyl optionally substituted with halogen, hydroxyl or C₁₋₃ alkyl, 5- to 10-membered heteroaryl optionally substituted with halogen, hydroxyl or C₁₋₃ alkyl, —SONHC₁₋₆ alkyl optionally substituted with halogen or hydroxyl, —SO₂C₁₋₆ alkyl optionally substituted with halogen or hydroxyl, —COC₁₋₆ alkyl optionally substituted with halogen or hydroxyl, —COC₃₋₆ cycloalkyl optionally substituted with halogen or hydroxyl, —COC₆₋₁₂ aryl optionally substituted with halogen or hydroxyl, —NHSO₂C₁₋₆ alkyl optionally substituted with halogen or hydroxyl, —CONHC₁₋₆ alkyl optionally substituted with halogen or hydroxyl, —NHC₁₋₆ alkyl optionally substituted with halogen or hydroxyl, —N(C₁₋₆ alkyl)₂ optionally substituted with halogen or hydroxyl, and —NHC₃₋₆ cycloalkyl optionally substituted with halogen or hydroxyl;
when the bond connecting Q and N is a double bond, the bond connecting N and Y is a single bond, in which case Q and Y are each selected from CR¹ or N; wherein R¹, at each occurrence, is independently selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
when the bond connecting N and Y is a double bond, the bond connecting Q and N is a single bond, in which case Y is selected from C, and Q is selected from CR²R³ or NR⁴; wherein R², R³ and R⁴, at each occurrence, are independently selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy and 4- to 6-membered heterocyclyl. the heteroatoms in the “heterocyclyl” and “heteroaryl” are selected from N, O or S, and the number of the heteroatoms is 1, 2, 3 or 4.

In a preferred embodiment of the present invention, the compound as shown in formula (A) is further represented by formula (A-1):

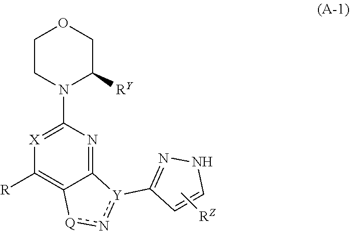

wherein the substituents in formula (A-1) are as defined in formula (A).

In a preferred embodiment of the present invention, the compound as shown in formula (A) is further represented by formula (A-2):

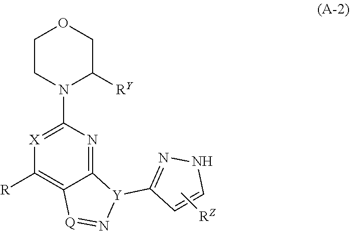

wherein the substituents in formula (A-2) are as defined in formula (A).

In a preferred embodiment of the present invention, the compound as shown in formula (A) is further represented by formula (A-3):

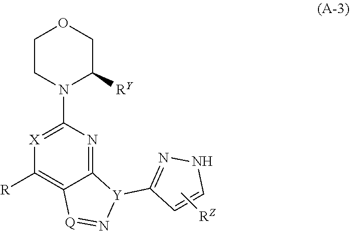

wherein the substituents in formula (A-3) are as defined in formula (A).

In a preferred embodiment of the present invention, the compound as shown in formula (A) is further represented by formula (A-4):

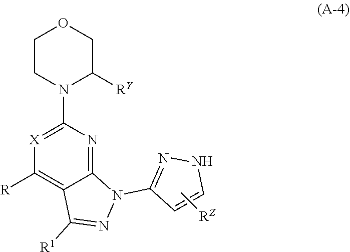

wherein the substituents in formula (A-4) are as defined in formula (A).

In a preferred embodiment of the present invention, the compound as shown in formula (A) is further represented by formula (A-5):

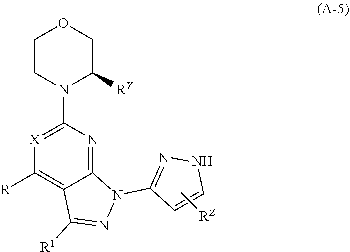

wherein the substituents in formula (A-5) are as defined in formula (A).

In a preferred embodiment of the present invention, the compound as shown in formula (A) is further represented by formula (A-6):

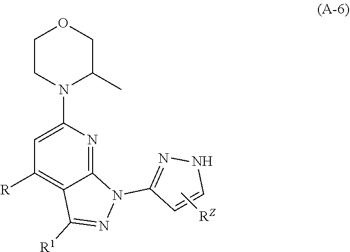

wherein the substituents in formula (A-6) are as defined in formula (A).

In a preferred embodiment of the present invention, the compound as shown in formula (A) is further represented by formula (A-7):

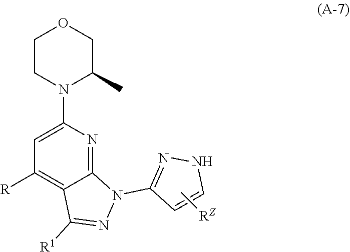

wherein the substituents in formula (A-7) are as defined in formula (A).

The present invention further provides a compound as shown in formula (I), and a stereoisomer, an optical isomer, a pharmaceutical salt, a prodrug and a solvate thereof,

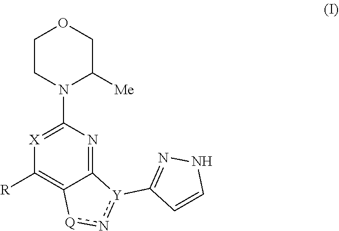

wherein,
one of the bond connecting Q and N and the bond connecting N and Y is a double bond; when the bond connecting Q and N is a double bond, the bond connecting N and Y is a single bond; when the bond connecting N and Y is a double bond, the bond connecting Q and N is a single bond;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R is selected from one of C₆₋₁₂ aryl, 5- to 12-membered heteroaryl, C₂₋₆ alkynyl, C₃₋₁₂ carbocyclyl and 3- to 12-membered heterocyclyl, wherein the C₆₋₁₂ aryl, 5- to 12-membered heteroaryl, C₂₋₆ alkynyl, C₃₋₁₂ carbocyclyl and 3- to 12-membered heterocyclyl are optionally substituted with one or more of the following substituents: hydroxyl, sulfhydryl, amino, carboxyl, cyano, halogen, oxo, aminoacyl, C₁₋₆ alkyl, C₂₋₆ alkenyl, C₂₋₆ alkynyl, C₃₋₁₂ carbocyclyl, C₆₋₁₂ aryl, C₁₋₆ alkoxy, C₁₋₆ alkylthio, 3- to 12-membered heterocyclyl, 5- to 12-membered heteroaryl, —C₁₋₆ alkyl-NH₂, —NHC₁₋₆ alkyl, —NH-5- to 12-membered heteroaryl, —N(C₁₋₆ alkyl)₂, —NHCOC₁₋₆ alkyl, —NHCOC₃₋₆ carbocyclyl, —NHCOC₃₋₁₂ aryl, —NHCO-3- to 12-membered heteroaryl, —NHCO-3- to 12-membered heterocyclyl, —NHCONHC₁₋₆ alkyl, —NHCONHC₃₋₁₂ carbocyclyl, —NHCONH-3- to 12-membered heterocyclyl, —CONH C₁₋₆ alkyl, —CON(C₁₋₆ alkyl)₂, —C₁₋₆ alkyl-C₃₋₁₂ carbocyclyl, —C₁₋₆ alkyl-5- to 12-membered heteroaryl, —C₁₋₆ alkyl-3- to 12-membered heterocyclyl and —C₁₋₆ alkyl-C₆₋₁₂ aryl, wherein the C₁₋₆ alkyl, C₂₋₆ alkenyl, C₂₋₆ alkynyl, C₃₋₁₂ carbocyclyl, C₆₋₁₂ aryl, C₁₋₆ alkoxy, C₁₋₆ alkylthio, 3- to 12-membered heterocyclyl, 5- to 12-membered heteroaryl, —C₁₋₆ alkyl-NH₂, —NHC₁₋₆ alkyl, —NH-5- to 12-membered heteroaryl, —N(C₁₋₆ alkyl)₂, —NHCOC₁₋₆ alkyl, —NHCOC₃₋₆ carbocyclyl, —NHCOC₃₋₁₂ aryl, —NHCO-3- to 12-membered heteroaryl, —NHCO-3- to 12-membered heterocyclyl, —NHCONHC₁₋₆ alkyl, —NHCONHC₃₋₆ carbocyclyl, —CONHC₁₋₆ alkyl, —CON(C₁₋₆ alkyl)₂, —C₁₋₆ alkyl-C₃₋₁₂ carbocyclyl, —C₁₋₆ alkyl-5- to 12-membered heteroaryl, —C₁₋₆ alkyl-3- to 12-membered heterocyclyl and —C₁₋₆ alkyl-C₆₋₁₂ aryl are optionally substituted with one or more of the following substituents: hydroxyl, sulfhydryl, amino, carboxyl, cyano, halogen, oxo, amido, aminoacyl, —SO₂NH₂, C₁₋₆ alkyl optionally substituted with halogen or hydroxyl, C₂₋₆ alkenyl optionally substituted with halogen or hydroxyl, C₂₋₆ alkynyl optionally substituted with halogen or hydroxyl, C₁₋₆ alkoxy optionally substituted with halogen or hydroxyl, —C₁₋₆ alkyl-OH optionally substituted with halogen or hydroxyl, —C₁₋₆ alkyl-O—C₁₋₆ alkyl optionally substituted with halogen or hydroxyl, C₃₋₆ cycloalkyl optionally substituted with halogen or hydroxyl, C₆₋₁₂ aryl optionally substituted with halogen or hydroxyl, 3- to 6-membered heterocyclyl optionally substituted with halogen or hydroxyl, 5- to 10-membered heteroaryl optionally substituted with halogen or hydroxyl, —SONHC₁₋₆ alkyl optionally substituted with halogen or hydroxyl, —SO₂C₁₋₆ alkyl optionally substituted with halogen or hydroxyl, —COC₁₋₆ alkyl optionally substituted with halogen or hydroxyl, —COC₃₋₆ cycloalkyl optionally substituted with halogen or hydroxyl, —COC₆₋₁₂ aryl optionally substituted with halogen or hydroxyl, —NHSO₂C₁₋₆ alkyl optionally substituted with halogen or hydroxyl, —CONHC₁₋₆ alkyl optionally substituted with halogen or hydroxyl, —NHC₁₋₆ alkyl optionally substituted with halogen or hydroxyl, —N(C₁₋₆ alkyl)₂ optionally substituted with halogen or hydroxyl, and —NHC₃₋₆ cycloalkyl optionally substituted with halogen or hydroxyl; when the bond connecting Q and N is a double bond, the bond connecting N and Y is a single bond, in which case Q and Y are each selected from CR¹ or N; wherein R¹, at each occurrence, is independently selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
when the bond connecting N and Y is a double bond, the bond connecting Q and N is a single bond, in which case Y is selected from C, and Q is selected from CR²R³ or NR⁴; wherein R², R³ and R⁴, at each occurrence, are independently selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy and 4- to 6-membered heterocyclyl. the heteroatoms in the “heterocyclyl” and “heteroaryl” are selected from N, O or S, and the number of the heteroatoms is 1, 2, 3 or 4.

In a preferred embodiment of the present invention, the compound as shown in formula (I) is further represented by formula (II):

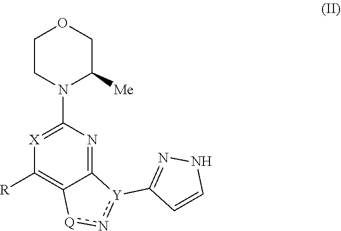

wherein the substituents in formula (II) are as defined in formula (I).

The present invention further provides a compound as shown in formula (B), and a stereoisomer, an optical isomer, a pharmaceutical salt, a prodrug and a solvate thereof,

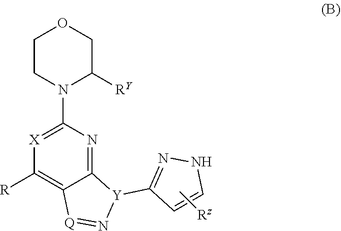

wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{Y} is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl;
R is selected from one of the following substituents:

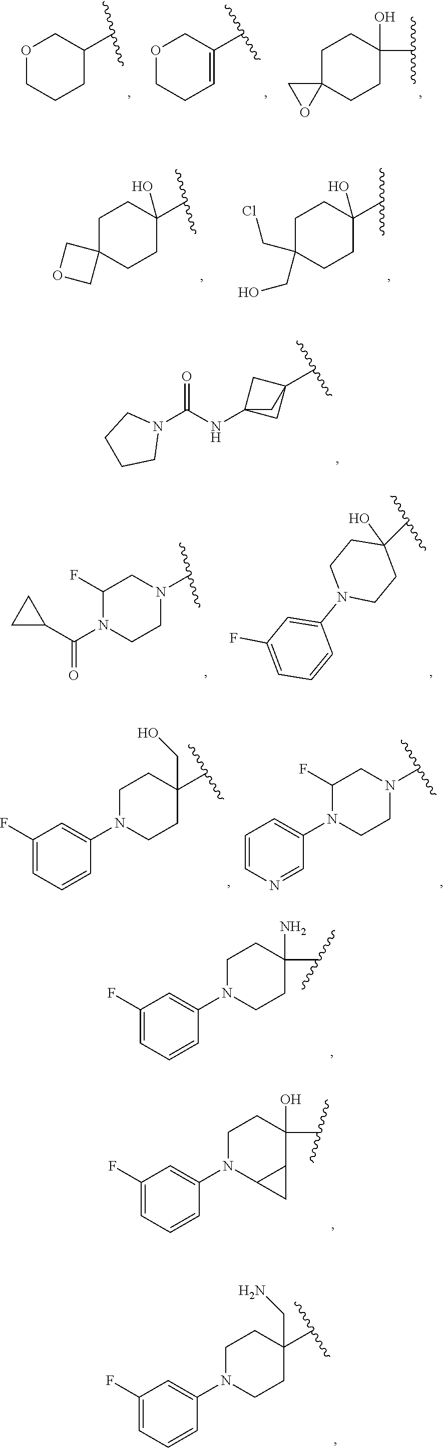

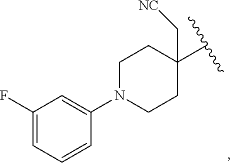 [SMILES: *C1(CC#N)CCN(c2cccc(F)c2)CC1]

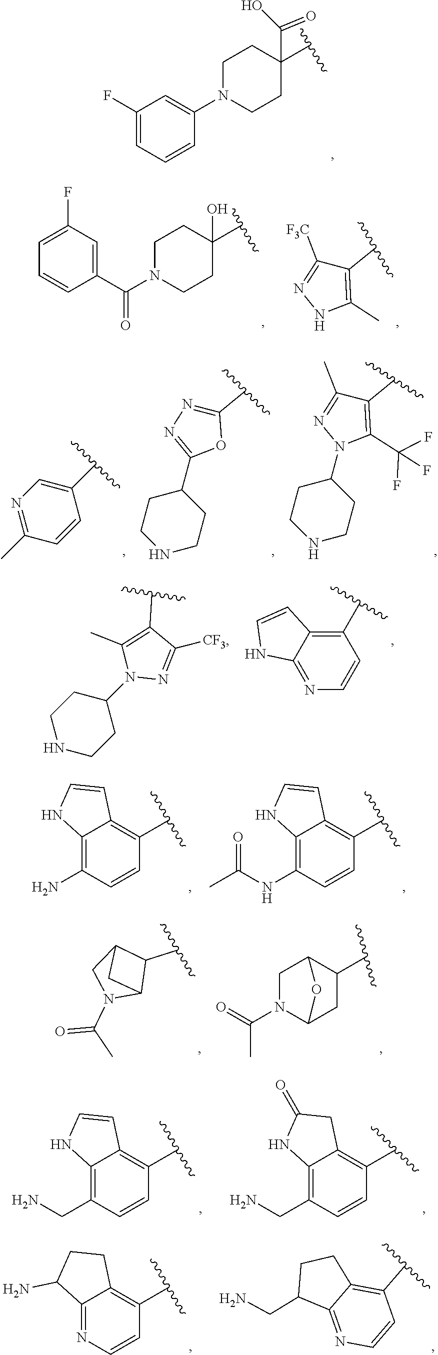

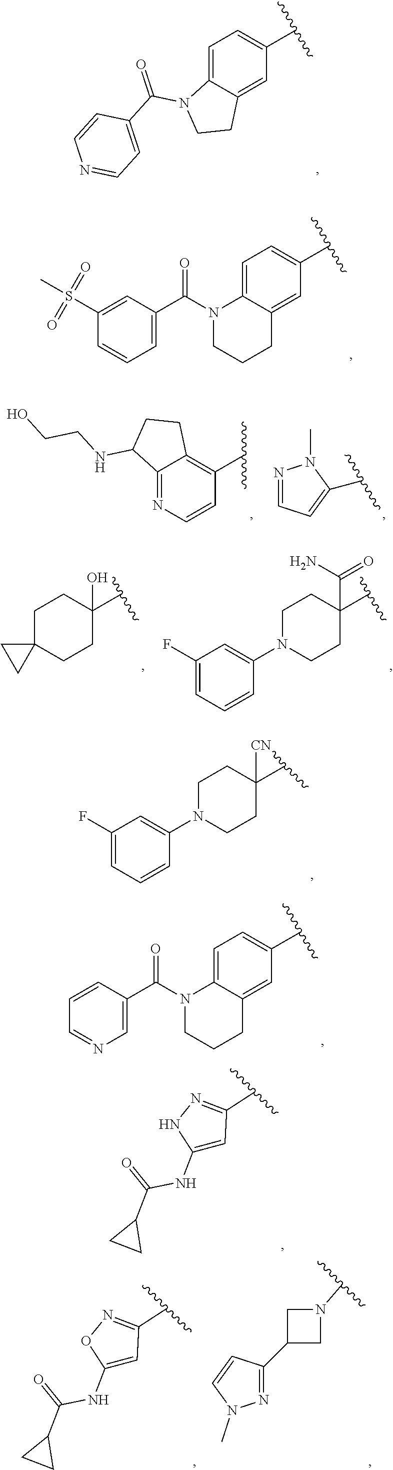

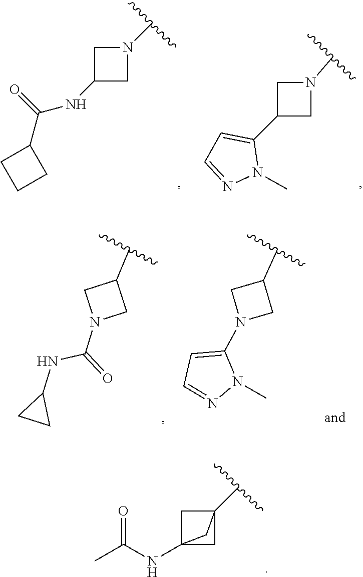

In preferred embodiments of the present invention, Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio;
further preferably, Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio;
further preferably, Y is selected from N, and Q is selected from CR¹; R¹ is selected from hydrogen, halogen, cyano, amido, methyl, ethyl, methoxy, ethoxy, methylthio or ethylthio; still further preferably, Y is selected from N, and Q is selected from CR¹; R¹ is selected from hydrogen, F, Cl, Br, cyano, amido, methyl, ethyl, methoxy or methylthio;
still further preferably, Y is selected from N, and Q is selected from CR¹; R¹ is selected from hydrogen, F, cyano, amido or methylthio;
most preferably, Y is selected from N, and Q is selected from CR¹; R¹ is selected from hydrogen.

In preferred embodiments of the present invention, X is selected from CR^{X}; wherein R^{X} is selected from hydrogen, halogen, hydroxyl, sulfhydryl, amino, cyano or C₁₋₆ alkyl; further preferably, R^{X} is selected from hydrogen, F, Cl, Br, hydroxyl, amino, cyano or methyl;
still further preferably, R^{X} is selected from hydrogen.

In preferred embodiments of the present invention, R¹ is F, Cl, Br, methyl, ethyl, n-propyl, isopropyl or hydrogen;
further preferably, R¹ is F, Cl, Br, methyl, ethyl or hydrogen;
still further preferably, R¹ is methyl.

In preferred embodiments of the present invention, the number of R^{Z} is 0, 1, 2 or 3, and R^{Z}, at each occurrence, is independently selected from F, Cl, Br, hydroxyl, cyano, amino, methyl, ethyl, monofluoromethyl, difluoromethyl or trifluoromethyl;
further preferably, the number of R^{Z} is 0, 1 or 2, and R^{Z}, at each occurrence, is independently selected from F, Cl, Br, hydroxyl, methyl, monofluoromethyl, difluoromethyl or trifluoromethyl;
still further preferably, the number of R^{Z} is 0.

In a preferred embodiment of the present invention, the compound as shown in formula (B) is further represented by formula (B-1):

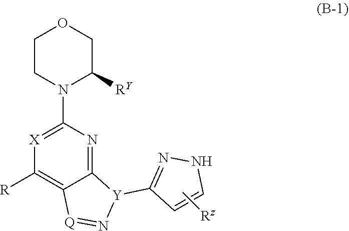

wherein the substituents in formula (B-1) are as defined in formula (B).

In a preferred embodiment of the present invention, the compound as shown in formula (B) is further represented by formula (B-2):

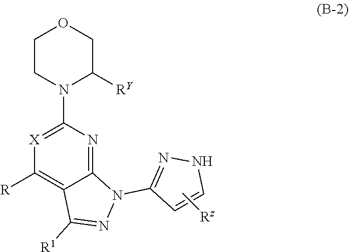

wherein the substituents in formula (B-2) are as defined in formula (B).

In a preferred embodiment of the present invention, the compound as shown in formula (B) is further represented by formula (B-3):

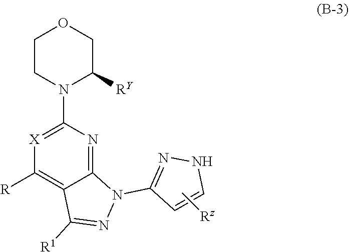

wherein the substituents in formula (B-3) are as defined in formula (B).

In a preferred embodiment of the present invention, the compound as shown in formula (B) is further represented by formula (B-4):

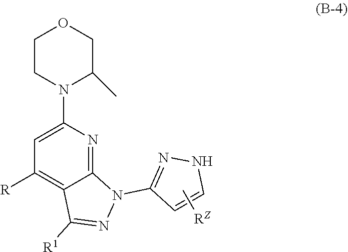

wherein the substituents in formula (B-4) are as defined in formula (B).

In a preferred embodiment of the present invention, the compound as shown in formula (B) is further represented by formula (B-5):

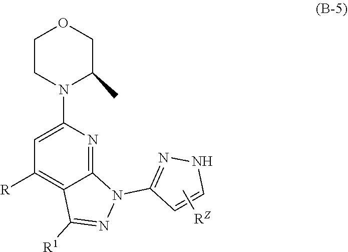

wherein the substituents in formula (B-5) are as defined in formula (B).

The present invention further provides a compound as shown in formula (III), and a stereoisomer, an optical isomer, a pharmaceutical salt, a prodrug and a solvate thereof,

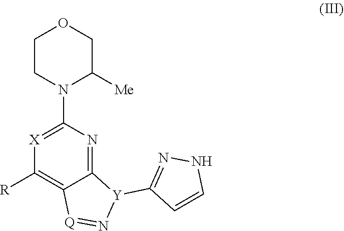

wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R is selected from one of the following substituents:

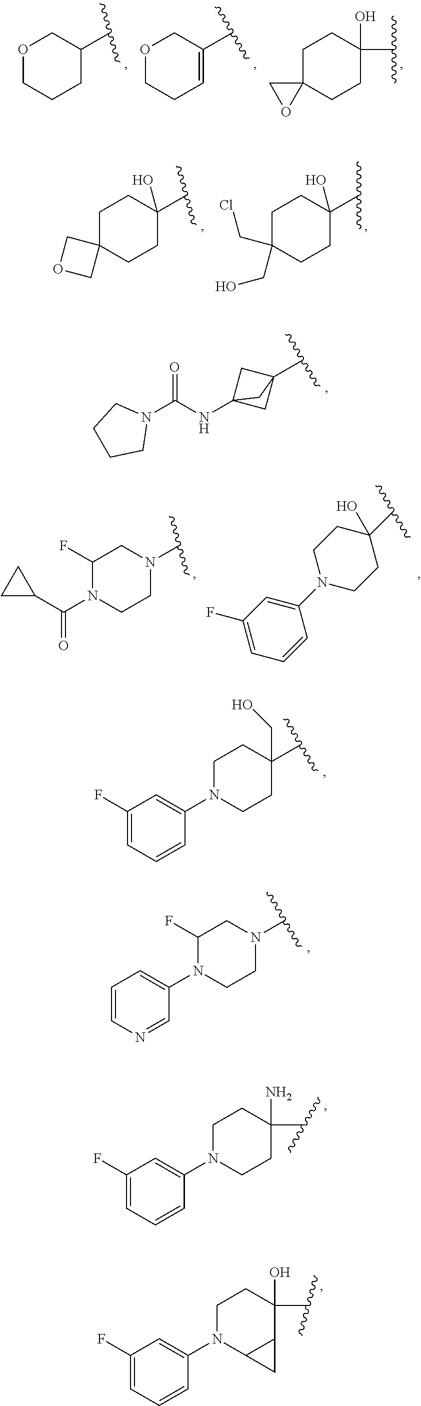

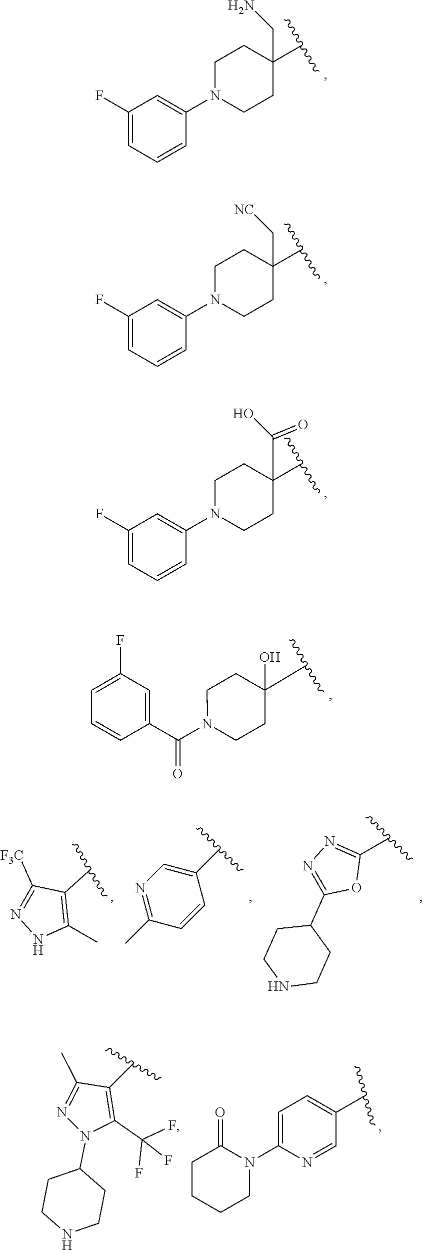

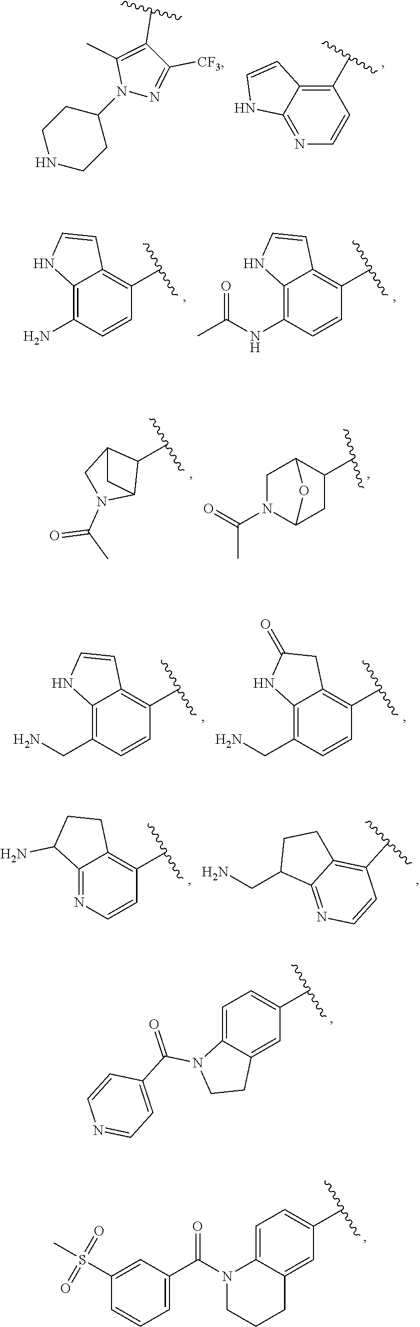

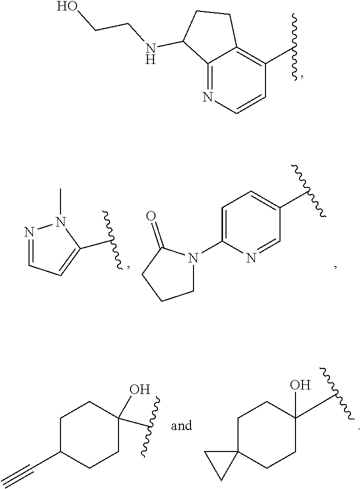

In preferred embodiments of the present invention, Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio;
further preferably, Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio;
further preferably, Y is selected from N, and Q is selected from CR¹; R¹ is selected from hydrogen, halogen, cyano, amido, methyl, ethyl, methoxy, ethoxy, methylthio or ethylthio;
still further preferably, Y is selected from N, and Q is selected from CR¹; R¹ is selected from hydrogen, F, Cl, Br, cyano, amido, methyl, ethyl, methoxy or methylthio;
still further preferably, Y is selected from N, and Q is selected from CR¹; R¹ is selected from hydrogen, F, cyano, amido or methylthio.

In preferred embodiments of the present invention, X is selected from CR^{X}; wherein R^{X} is selected from hydrogen, halogen, hydroxyl, sulfhydryl, amino, cyano or C₁₋₆ alkyl; further preferably, R^{X} is selected from hydrogen, F, Cl, Br, hydroxyl, amino, cyano or methyl; still further preferably, R^{X} is selected from hydrogen.

In a preferred embodiment of the present invention, the compound as shown in formula (III) is further represented by formula (IV):

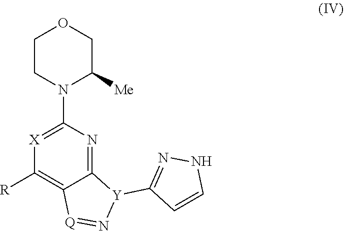

wherein the substituents in formula (IV) are as defined in formula (III).

The present invention further provides a compound as shown in formula (C), and a stereoisomer, an optical isomer, a pharmaceutical salt, a prodrug and a solvate thereof,

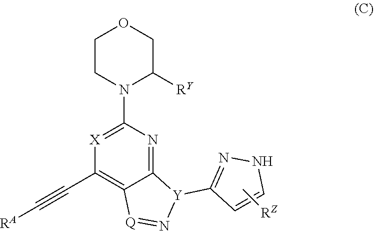

wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{Y} is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl;
R^{A} is selected from hydrogen, carboxyl, —C₁₋₆ alkyl-NH₂, —Z—C₁₋₆ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl or —CONHC₁₋₆ alkyl; wherein —Z— is selected from a bond, —C(R¹⁰)(R¹¹)—, —C(R¹²)(R¹³) C(R¹⁴)(R¹⁵)—, —N(R¹⁶)—, —O— or —S—, wherein R¹⁰, R¹¹, R¹², R¹³, R¹⁴, R¹⁵ and R¹⁶ are each independently selected from hydrogen, methyl, ethyl, hydroxyl, carboxyl, amino, amido, cyano and oxo, and when one substituent of R¹⁰ and R¹¹, R¹² and R¹³, or R¹⁴ and R¹⁵ connected to the same atom is selected from oxo, the other substituent is absent; the —C₁₋₆ alkyl-NH₂, —Z—C₁₋₆ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl and —CONHC₁₋₆ alkyl are optionally substituted with one or more of the following substituents: hydroxyl, cyano, halogen, oxo, amido, —SO₂NH₂, optionally substituted C₁₋₆ alkyl, optionally substituted C₁₋₆ alkoxy, optionally substituted —C₁₋₆ hydroxyalkyl, optionally substituted C₆₋₁₂ aryl, optionally substituted 3- to 6-membered heterocyclyl, optionally substituted 5- to 10-membered heteroaryl, optionally substituted —SONHC₁₋₆ alkyl, optionally substituted —SO₂C₁₋₆ alkyl, optionally substituted —COC₁₋₆ alkyl, optionally substituted —COC₃₋₆ cycloalkyl, optionally substituted —COC₆₋₁₂ aryl, optionally substituted —NHSO₂C₁₋₆ alkyl, and optionally substituted —CONHC₁₋₆ alkyl; the expression “optionally substituted” refers to the case of being unsubstituted or substituted with one or more of the following substituents: C₁₋₆ alkyl, hydroxyl, halogen and oxo;
the heteroatoms in the “heterocyclyl” and “heteroaryl” are selected from N, O or S, and the number of the heteroatoms is 1, 2, 3 or 4.

In preferred embodiments of the present invention, Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio;
further preferably, Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio;
further preferably, Y is selected from N, and Q is selected from CR¹; R¹ is selected from hydrogen, halogen, cyano, amido, methyl, ethyl, methoxy, ethoxy, methylthio or ethylthio;
still further preferably, Y is selected from N, and Q is selected from CR¹; R¹ is selected from hydrogen, F, Cl, Br, cyano, amido, methyl, ethyl, methoxy or methylthio;
still further preferably, Y is selected from N, and Q is selected from CR¹; R¹ is selected from hydrogen, F, cyano, amido or methylthio;
most preferably, Y is selected from N, and Q is selected from CR¹; R¹ is selected from hydrogen.

In preferred embodiments of the present invention, X is selected from CR^{X}; wherein R^{X} is selected from hydrogen, halogen, hydroxyl, sulfhydryl, amino, cyano or C₁₋₆ alkyl; further preferably, R^{X} is selected from hydrogen, F, Cl, Br, hydroxyl, amino, cyano or methyl;
still further preferably, R^{X} is selected from hydrogen.

In preferred embodiments of the present invention, R^{Y} is F, Cl, Br, methyl, ethyl, n-propyl, isopropyl or hydrogen;
further preferably, R^{Y} is F, Cl, Br, methyl, ethyl or hydrogen;
still further preferably, R^{Y} is methyl.

In preferred embodiments of the present invention, the number of R^{Z} is 0, 1, 2 or 3, and R^{Z}, at each occurrence, is independently selected from F, Cl, Br, hydroxyl, cyano, amino, methyl, ethyl, monofluoromethyl, difluoromethyl or trifluoromethyl;
further preferably, the number of R^{Z} is 0, 1 or 2, and R^{Z}, at each occurrence, is independently selected from F, Cl, Br, hydroxyl, methyl, monofluoromethyl, difluoromethyl or trifluoromethyl;
still further preferably, the number of R^{Z} is 0.

In preferred embodiments of the present invention, R^{A} is selected from hydrogen, carboxyl, amido, —C₁₋₄ alkyl-NH₂, —Z—C₁₋₄ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl or —CONHC₁₋₄ alkyl; wherein —Z— is selected from a bond, —C(R¹⁰)(R¹¹)—, —C(R¹²)(R¹³) C(R¹⁴)(R¹⁵)— or —N(R¹⁶)—, wherein R¹⁰, R¹¹, R¹², R¹³, R¹⁴, R¹⁵ and R¹⁶ are each independently selected from hydrogen, methyl, hydroxyl, amino, cyano and oxo, and when one substituent of R¹⁰ and R¹¹, R¹² and R¹³, or R¹⁴ and R¹⁵ connected to the same atom is selected from oxo, the other substituent is absent; the amido, —C₁₋₄ alkyl-NH₂, —Z—C₁₋₄ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl and —CONHC₁₋₄ alkyl are optionally substituted with one or more of the following substituents: hydroxyl, cyano, halogen, oxo, amido, —SO₂NH₂, optionally substituted C₁₋₄ alkyl, optionally substituted C₁₋₄ alkoxy, optionally substituted —C₁₋₄ alkyl-OH, optionally substituted C₆₋₁₂ aryl, optionally substituted 3- to 6-membered heterocyclyl, optionally substituted 5- to 10-membered heteroaryl, optionally substituted —SONHC₁₋₄ alkyl, optionally substituted —SO₂C₁₋₄ alkyl, optionally substituted —COC₁₋₄ alkyl, optionally substituted —COC₃₋₆ cycloalkyl, optionally substituted —COC₆₋₁₂ aryl, optionally substituted —NHSO₂C₁₋₄ alkyl, and optionally substituted —CONHC₁₋₄ alkyl; the expression “optionally substituted” refers to the case of being unsubstituted or substituted with one or more of the following substituents: methyl, ethyl, n-propyl, isopropyl, hydroxyl, halogen and oxo;
further preferably, R^{A} is selected from hydrogen, carboxyl, amido, —C₁₋₄ alkyl-NH₂, —Z—C₁₋₄ alkyl, —Z—C₃₋₆ monocyclic cycloalkyl, —Z-6- to 8-membered spirocycloalkyl, —Z—C₆ cycloalkenyl, —Z-phenyl, —Z-4- to 7-membered monocyclic heterocyclyl, —Z-6- to 8-membered bridged heterocyclyl, —Z-6- to 9-membered fused heterocyclyl, —Z-7- to 11-membered spiro heterocyclyl, —Z-5- to 6-membered monocyclic heteroaryl or —Z-7- to 9-membered fused heteroaryl, wherein —Z— is selected from a bond, —CH₂—, —CH₂CH₂—, —NH—, —CH(OH)—, —CH(CN)—, —CH(CH₃)—, —CO—, —COCH₂—, —CH₂CO—, —COCO—, —CH(OH)CH₂—, —CH₂CH(OH)—, —CH(CN)CH₂—, —CH₂CH(CN)—, —CH(CH₃)CH₂—, —CH₂CH(CH₃)—, —CONH— or —CON(CH₃)—; the amido, C₁₋₄ alkyl-NH₂, —Z—C₁₋₄ alkyl, —Z—C₃₋₆ monocyclic cycloalkyl, —Z-6- to 8-membered spirocycloalkyl, —Z—C₆ cycloalkenyl, —Z-phenyl, —Z-4- to 7-membered monocyclic heterocyclyl, —Z-6- to 8-membered bridged heterocyclyl, —Z-6- to 9-membered fused heterocyclyl, —Z-7- to 11-membered spiro heterocyclyl, —Z-5- to 6-membered monocyclic heteroaryl and —Z-7- to 9-membered fused heteroaryl are optionally substituted with one or more of the following substituents: hydroxyl, cyano, halogen, oxo, amido, —SO₂NH₂, optionally substituted methyl, optionally substituted ethyl, optionally substituted n-propyl, optionally substituted isopropyl, optionally substituted methoxy, optionally substituted ethoxy, optionally substituted hydroxymethyl, optionally substituted hydroxyethyl, optionally substituted phenyl, optionally substituted 5- to 6-membered heterocyclyl, optionally substituted 5- to 6-membered heteroaryl, optionally substituted —SONHCH₃, optionally substituted —SO₂CH₃, optionally substituted —COCH₃, optionally substituted —COCH₂CH₃, optionally substituted —COC₃₋₆ cycloalkyl, optionally substituted —CO-phenyl, optionally substituted —NHSO₂CH₃, and optionally substituted —CONHCH₃; the expression “optionally substituted” refers to the case of being unsubstituted or substituted with one or more of the following substituents: methyl, ethyl, hydroxyl, halogen and oxo;
further preferably, R^{A} is selected from hydrogen, carboxyl, or the following substituents which are optionally substituted: amido, —Z-methyl, —Z-ethyl, —Z-n-propyl, —Z-isopropyl, —Z-cyclopropyl, —Z-cyclobutyl, —Z-cyclopentyl, —Z-cyclohexyl, —Z—C₆ cycloalkenyl, —Z—C₆/C₃ spirocycloalkyl, —Z-5-membered monocyclic heterocyclyl, —Z-6-membered monocyclic heterocyclyl, —Z-7-membered monocyclic heterocyclyl, —Z-5-membered monocyclic heteroaryl, —Z-6-membered monocyclic heteroaryl, —Z-phenyl, —Z-7-membered bridged heterocyclyl, —Z-8-membered bridged heterocyclyl, —Z-6-membered/3-membered fused heterocyclyl, —Z-6-membered/4-membered fused heterocyclyl, —Z-6-membered/5-membered fused heterocyclyl, —Z-3-membered/6-membered fused heterocyclyl, —Z-4-membered/6-membered fused heterocyclyl, —Z-5-membered/6-membered fused heterocyclyl, —Z-3-membered/4-membered fused heterocyclyl, —Z-4-membered/3-membered fused heterocyclyl, —Z-5-membered/3-membered fused heterocyclyl, —Z-3-membered/5-membered fused heterocyclyl, —Z-5-membered/4-membered fused heterocyclyl, —Z-4-membered/5-membered fused heterocyclyl, —Z-5-membered/5-membered fused heterocyclyl, —Z-4-membered/4-membered fused heterocyclyl, —Z-4-membered/4-membered spiro heterocyclyl, —Z-5-membered/4-membered spiro heterocyclyl, —Z-4-membered/5-membered spiro heterocyclyl, —Z-5-membered/5-membered spiro heterocyclyl, —Z-4-membered/6-membered spiro heterocyclyl, —Z-6-membered/4-membered spiro heterocyclyl, —Z-5-membered/6-membered spiro heterocyclyl, —Z-6-membered/5-membered spiro heterocyclyl, —Z-6-membered/6-membered spiro heterocyclyl, —Z-5-membered/5-membered fused heteroaryl, —Z-5-membered/6-membered fused heteroaryl, —Z-6-membered/5-membered fused heteroaryl, -methyl-NH₂, -ethyl-NH₂, —CONHCH₃ and —CONHCH₂CH₃, wherein —Z— is selected from a bond, —CH₂—, —CH₂CH₂—, —NH— or —CONH—; the expression “optionally substituted” refers to the case of being unsubstituted or substituted with one or more of the following substituents: hydroxyl, cyano, halogen, oxo, amido, —SO₂NH₂, methyl, ethyl, n-propyl, isopropyl, halomethyl, haloethyl, halo n-propyl, halo isopropyl, methoxy, ethoxy, hydroxymethyl, hydroxyethyl, phenyl, benzyl, halophenyl, 5- to 6-membered heterocyclyl, 5- to 6-membered heterocyclyl substituted with methyl, halo 5- to 6-membered heterocyclyl, 5- to 6-membered heteroaryl, 5- to 6-membered heteroaryl substituted with methyl, halo 5- to 6-membered heteroaryl, —SONHCH₃, —SO₂CH₃, —COCH₃, —COCH₂CH₃, —COC₃₋₆ cycloalkyl, —CO-phenyl, —NHSO₂CH₃ and —CONHCH₃;
further preferably, R^{A} is selected from hydrogen, carboxyl, or the following substituents which are optionally substituted: methyl, ethyl, n-propyl, isopropyl, cyclopropyl, cyclobutyl, cyclopentyl, cyclohexyl, methylamino, ethylamino, phenyl, amido,

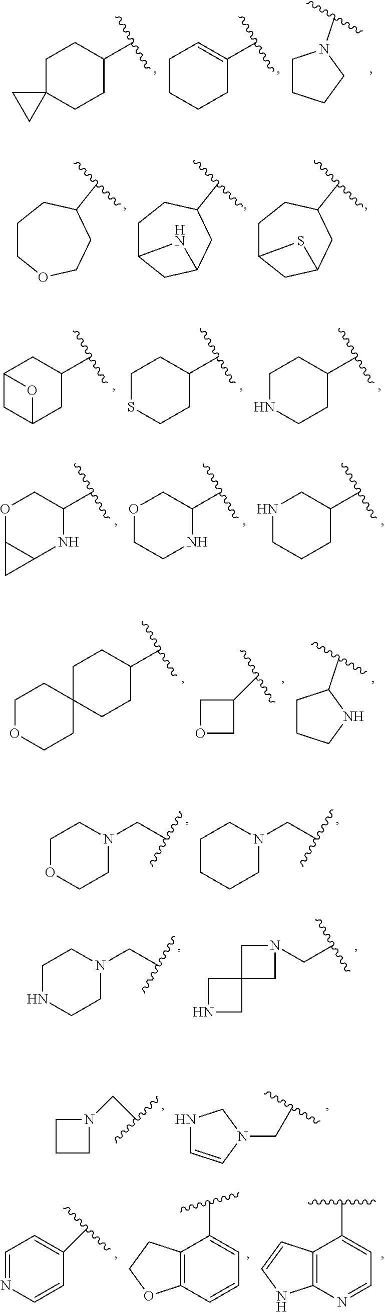

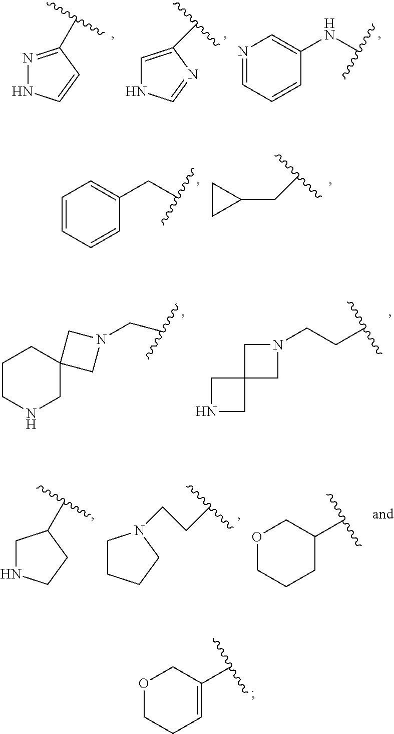

the expression “optionally substituted” refers to the case of being unsubstituted or substituted with one or more of the substituents selected from: hydroxyl, cyano, F, Cl, Br, oxo, amido, —SO₂NH₂, methyl, ethyl, n-propyl, isopropyl, monofluoromethyl, difluoromethyl, trifluoromethyl, methoxy, hydroxymethyl, benzyl, phenyl optionally substituted with methyl or halogen, pyridyl optionally substituted with methyl or halogen, pyrazolyl optionally substituted with methyl or halogen, —SONHCH₃, —SO₂CH₃, —COCH₃, —COCH₂CH₃, —CO-cyclopropyl, —CO-cyclobutyl, —CO— cyclopentyl, —CO-phenyl, —NHSO₂CH₃ and —CONHCH₃; further preferably, R^{A} is selected from one of hydrogen, carboxyl,

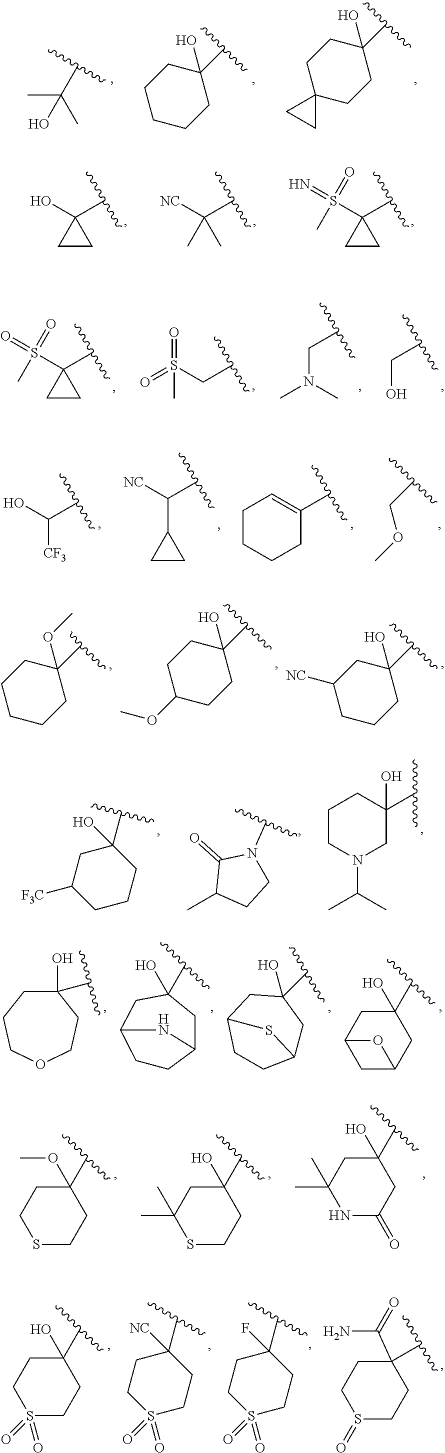

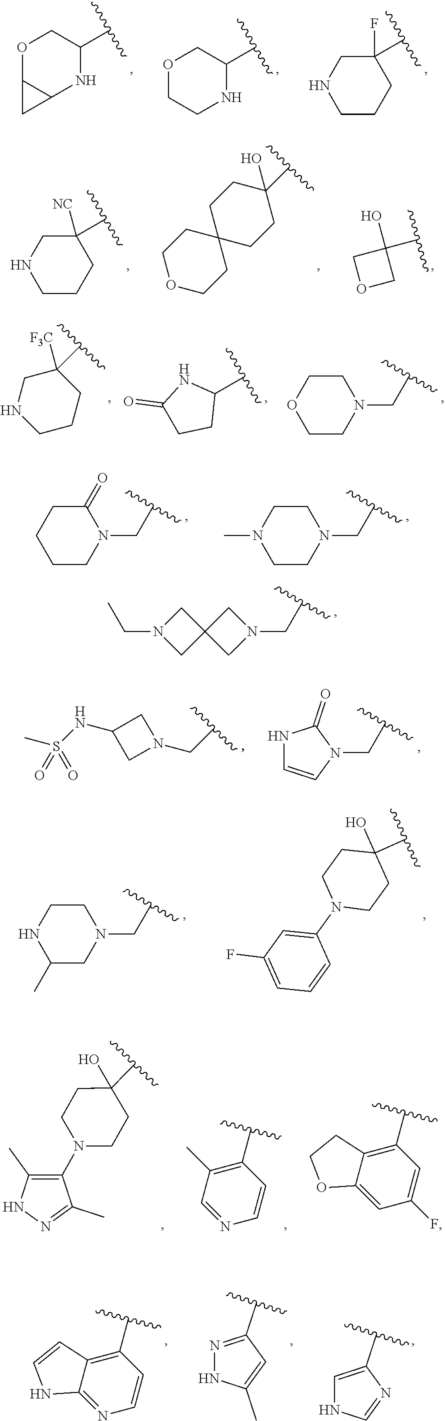

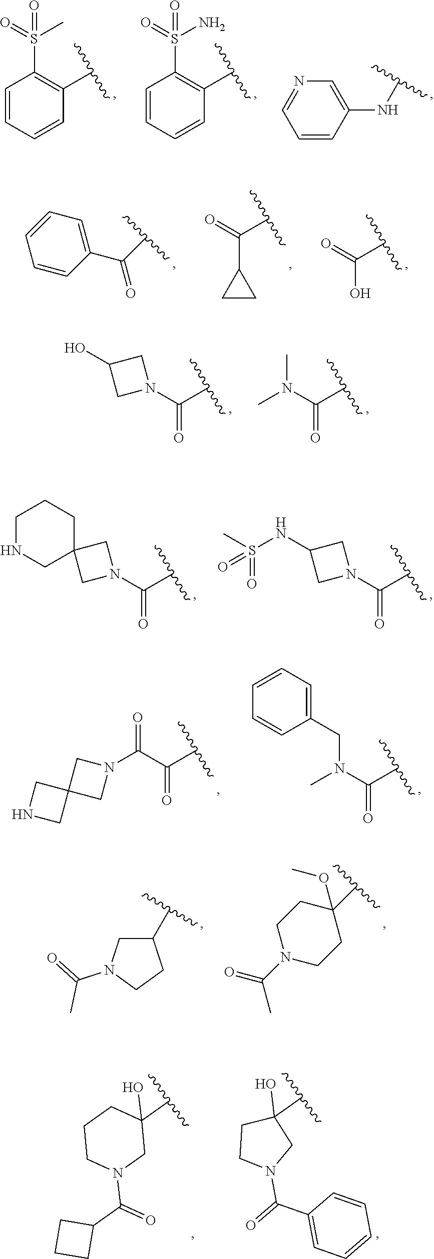

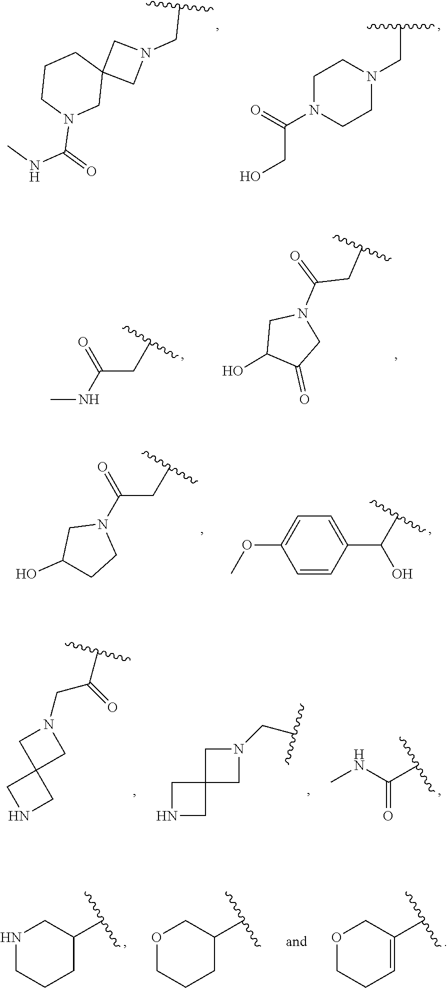

In a preferred embodiment of the present invention, the compound as shown in formula (C) is further represented by formula (C-1):

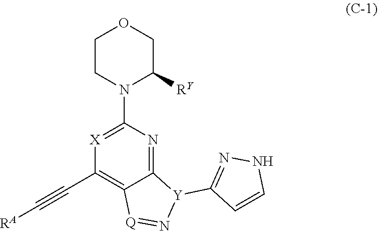

wherein the substituents in formula (C-1) are as defined in formula (C).

In a preferred embodiment of the present invention, the compound as shown in formula (C) is further represented by formula (C-2):

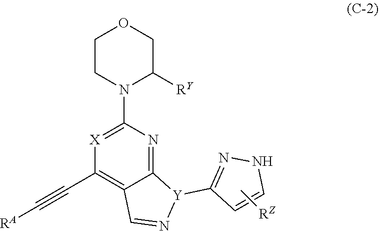

wherein the substituents in formula (C-2) are as defined in formula (C).

In a preferred embodiment of the present invention, the compound as shown in formula (C) is further represented by formula (C-3):

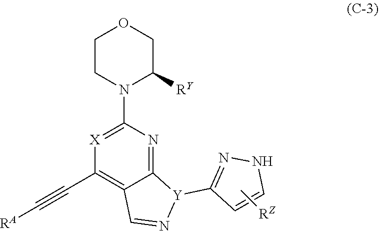

wherein the substituents in formula (C-3) are as defined in formula (C).

In a preferred embodiment of the present invention, the compound as shown in formula (C) is further represented by formula (C-4):

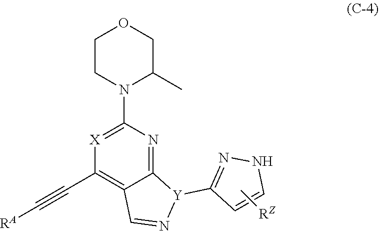

wherein the substituents in formula (C-4) are as defined in formula (C).

In a preferred embodiment of the present invention, the compound as shown in formula (C) is further represented by formula (C-5):

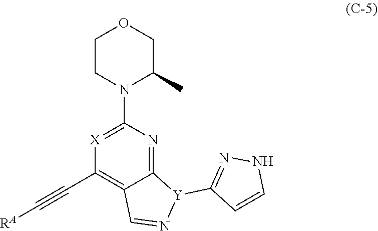

wherein the substituents in formula (C-5) are as defined in formula (C).

The present invention further provides a compound as shown in formula (V), and a stereoisomer, an optical isomer, a pharmaceutical salt, a prodrug and a solvate thereof,

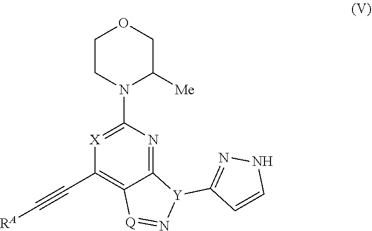

wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{A} is selected from hydrogen, carboxyl, —C₁₋₆ alkyl-NH₂, —Z—C₁₋₆ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl or —CONHC₁₋₆ alkyl; wherein —Z— is selected from a bond, —C(R¹⁰)(R¹¹)—, —C(R¹²)(R¹³) C(R¹⁴)(R¹⁵)—, —N(R¹⁶)—, —O— or —S—, wherein R¹⁰, R¹¹, R¹², R¹³, R¹⁴, R¹⁵ and R¹⁶ are each independently selected from hydrogen, methyl, ethyl, hydroxyl, carboxyl, amino, amido, cyano and oxo, and when one substituent of R¹⁰ and R¹¹, R¹² and R¹³, or R¹⁴ and R¹⁵ connected to the same atom is selected from oxo, the other substituent is absent; the —C₁₋₆ alkyl-NH₂, —Z—C₁₋₆ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl and —CONHC₁₋₆ alkyl are optionally substituted with one or more of the following substituents: hydroxyl, cyano, halogen, oxo, amido, —SO₂NH₂, optionally substituted C₁₋₆ alkyl, optionally substituted C₁₋₆ alkoxy, optionally substituted —C₁₋₆ alkylhydroxyl, optionally substituted C₆₋₁₂ aryl, optionally substituted 3- to 6-membered heterocyclyl, optionally substituted 5- to 10-membered heteroaryl, optionally substituted —SONHC₁₋₆ alkyl, optionally substituted —SO₂C₁₋₆ alkyl, optionally substituted —COC₁₋₆ alkyl, optionally substituted —COC₃₋₆ cycloalkyl, optionally substituted —COC₆₋₁₂ aryl, optionally substituted —NHSO₂C₁₋₆ alkyl, and optionally substituted —CONHC₁₋₆ alkyl; the expression “optionally substituted” refers to the case of being unsubstituted or substituted with one or more of the following substituents: C₁₋₆ alkyl, hydroxyl, halogen and oxo;
the heteroatoms in the “heterocyclyl” and “heteroaryl” are selected from N, O or S, and the number of the heteroatoms is 1, 2, 3 or 4.

In preferred embodiments of the present invention, Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio;
further preferably, Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio;
further preferably, Y is selected from N, and Q is selected from CR¹; R¹ is selected from hydrogen, halogen, cyano, amido, methyl, ethyl, methoxy, ethoxy, methylthio or ethylthio;
still further preferably, Y is selected from N, and Q is selected from CR¹; R¹ is selected from hydrogen, F, Cl, Br, cyano, amido, methyl, ethyl, methoxy or methylthio;
still further preferably, Y is selected from N, and Q is selected from CR¹; R¹ is selected from hydrogen, F, cyano, amido or methylthio.

In preferred embodiments of the present invention, X is selected from CR^{X}; wherein R^{X} is selected from hydrogen, halogen, hydroxyl, sulfhydryl, amino, cyano or C₁₋₆ alkyl;
further preferably, R^{X} is selected from hydrogen, F, Cl, Br, hydroxyl, amino, cyano or methyl;
still further preferably, R^{X} is selected from hydrogen.

In preferred embodiments of the present invention, R^{A} is selected from hydrogen, carboxyl, —C₁₋₄ alkyl-NH₂, —Z—C₁₋₄ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl or —CONHC₁₋₄ alkyl; wherein —Z— is selected from a bond, —C(R¹⁰)(R¹¹)—, —C(R¹²)(R¹³) C(R¹⁴)(R¹⁵)— or —N(R¹⁶)—, wherein R¹⁰, R¹¹, R¹², R¹³, R¹⁴, R¹⁵ and R¹⁶ are each independently selected from hydrogen, methyl, hydroxyl, amino, cyano and oxo, and when one substituent of R¹⁰ and R¹¹, R¹² and R¹³, or R¹⁴ and R¹⁵ connected to the same atom is selected from oxo, the other substituent is absent; the —C₁₋₄ alkyl-NH₂, —Z—C₁₋₄ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl and —CONHC₁₋₄ alkyl are optionally substituted with one or more of the following substituents: hydroxyl, cyano, halogen, oxo, amido, —SO₂NH₂, optionally substituted C₁. 4 alkyl, optionally substituted C₁₋₄ alkoxy, optionally substituted —C₁₋₄ alkyl-OH, optionally substituted C₆₋₁₂ aryl, optionally substituted 3- to 6-membered heterocyclyl, optionally substituted 5- to 10-membered heteroaryl, optionally substituted —SONHC₁₋₄ alkyl, optionally substituted —SO₂C₁₋₄ alkyl, optionally substituted —COC₁₋₄ alkyl, optionally substituted —COC₃₋₆ cycloalkyl, optionally substituted —COC₆₋₁₂ aryl, optionally substituted —NHSO₂C₁₋₄ alkyl, and optionally substituted —CONHC₁₋₄ alkyl; the expression “optionally substituted” refers to the case of being unsubstituted or substituted with one or more of the following substituents: methyl, ethyl, n-propyl, isopropyl, hydroxyl, halogen and oxo;
further preferably, R^{A} is selected from hydrogen, carboxyl, —C₁₋₄ alkyl-NH₂, —Z—C₁₋₄ alkyl, —Z—C₃₋₆ monocyclic cycloalkyl, —Z-6- to 8-membered spirocycloalkyl, —Z—C₆ cycloalkenyl, —Z-phenyl, —Z-4- to 7-membered monocyclic heterocyclyl, —Z-6- to 8-membered bridged heterocyclyl, —Z-6- to 9-membered fused heterocyclyl, —Z-7- to 11-membered spiro heterocyclyl, —Z-5- to 6-membered monocyclic heteroaryl or —Z-7- to 9-membered fused heteroaryl, wherein —Z— is selected from a bond, —CH₂—, —CH₂CH₂—, —NH—, —CH(OH)—, —CH(CN)—, —CH(CH₃)—, —CO—, —COCH₂—, —CH₂CO—, —COCO—, —CH(OH)CH₂—, —CH₂CH(OH)—, —CH(CN)CH₂—, —CH₂CH(CN)—, —CH(CH₃)CH₂—, —CH₂CH(CH₃)—, —CONH— or —CON(CH₃)—; the C₁₋₄ alkyl-NH₂, —Z—C₁₋₄ alkyl, —Z—C₃₋₆ monocyclic cycloalkyl, —Z-6- to 8-membered spirocycloalkyl, —Z—C₆ cycloalkenyl, —Z-phenyl, —Z-4- to 7-membered monocyclic heterocyclyl, —Z-6-to 8-membered bridged heterocyclyl, —Z-6- to 9-membered fused heterocyclyl, —Z-7- to 11-membered spiro heterocyclyl, —Z-5- to 6-membered monocyclic heteroaryl and —Z-7- to 9-membered fused heteroaryl are optionally substituted with one or more of the following substituents: hydroxyl, cyano, halogen, oxo, amido, —SO₂NH₂, optionally substituted methyl, optionally substituted ethyl, optionally substituted n-propyl, optionally substituted isopropyl, optionally substituted methoxy, optionally substituted ethoxy, optionally substituted hydroxymethyl, optionally substituted hydroxyethyl, optionally substituted phenyl, optionally substituted 5- to 6-membered heterocyclyl, optionally substituted 5- to 6-membered heteroaryl, optionally substituted —SONHCH₃, optionally substituted —SO₂ CH₃, optionally substituted —COCH₃, optionally substituted —COCH₂CH₃, optionally substituted —COC₃₋₆ cycloalkyl, optionally substituted —CO-phenyl, optionally substituted —NHSO₂CH₃, and optionally substituted —CONHCH₃; the expression “optionally substituted” refers to the case of being unsubstituted or substituted with one or more of the following substituents: methyl, ethyl, hydroxyl, halogen and oxo;
further preferably, R^{A} is selected from hydrogen, carboxyl, or the following substituents which are optionally substituted: —Z-methyl, —Z-ethyl, —Z-n-propyl, —Z-isopropyl, —Z-cyclopropyl, —Z— cyclobutyl, —Z-cyclopentyl, —Z-cyclohexyl, —Z—C₆ cycloalkenyl, —Z—C₆/C₃ spirocycloalkyl, —Z-5-membered monocyclic heterocyclyl, —Z-6-membered monocyclic heterocyclyl, —Z-7-membered monocyclic heterocyclyl, —Z-5-membered monocyclic heteroaryl, —Z-6-membered monocyclic heteroaryl, —Z-phenyl, —Z-7-membered bridged heterocyclyl, —Z-8-membered bridged heterocyclyl, —Z-6-membered/3-membered fused heterocyclyl, —Z-6-membered/4-membered fused heterocyclyl, —Z-6-membered/5-membered fused heterocyclyl, —Z-3-membered/6-membered fused heterocyclyl, —Z-4-membered/6-membered fused heterocyclyl, —Z-5-membered/6-membered fused heterocyclyl, —Z-3-membered/4-membered fused heterocyclyl, —Z-4-membered/3-membered fused heterocyclyl, —Z-5-membered/3-membered fused heterocyclyl, —Z-3-membered/5-membered fused heterocyclyl, —Z-5-membered/4-membered fused heterocyclyl, —Z-4-membered/5-membered fused heterocyclyl, —Z-5-membered/5-membered fused heterocyclyl, —Z-4-membered/4-membered fused heterocyclyl, —Z-5-membered/4-membered spiro heterocyclyl, —Z-4-membered/5-membered spiro heterocyclyl, —Z-5-membered/5-membered spiro heterocyclyl, —Z-4-membered/6-membered spiro heterocyclyl, —Z-6-membered/4-membered spiro heterocyclyl, —Z-5-membered/6-membered spiro heterocyclyl, —Z-6-membered/5-membered spiro heterocyclyl, —Z-6-membered/6-membered spiro heterocyclyl, —Z-5-membered/5-membered fused heteroaryl, —Z-5-membered/6-membered fused heteroaryl, —Z-6-membered/5-membered fused heteroaryl, -methyl-NH₂, -ethyl-NH₂, —CONHCH₃ and —CONHCH₂CH₃, wherein —Z— is selected from a bond, —CH₂—, —CH₂CH₂—, —NH— or —CONH—; the expression “optionally substituted” refers to the case of being unsubstituted or substituted with one or more of the following substituents: hydroxyl, cyano, halogen, oxo, amido, —SO₂NH₂, methyl, ethyl, n-propyl, isopropyl, halomethyl, haloethyl, halo n-propyl, halo isopropyl, methoxy, ethoxy, hydroxymethyl, hydroxyethyl, phenyl, benzyl, halophenyl, 5- to 6-membered heterocyclyl, 5- to 6-membered heterocyclyl substituted with methyl, halo 5- to 6-membered heterocyclyl, 5- to 6-membered heteroaryl, 5- to 6-membered heteroaryl substituted with methyl, halo 5- to 6-membered heteroaryl, —SONHCH₃, —SO₂CH₃, —COCH₃, —COCH₂CH₃, —COC₃₋₆ cycloalkyl, —CO-phenyl, —NHSO₂CH₃ and —CONHCH₃;
further preferably, R^{A} is selected from hydrogen, carboxyl, or the following substituents which are optionally substituted: methyl, ethyl, n-propyl, isopropyl, cyclopropyl, cyclobutyl, cyclopentyl, cyclohexyl, methylamino, ethylamino, phenyl,

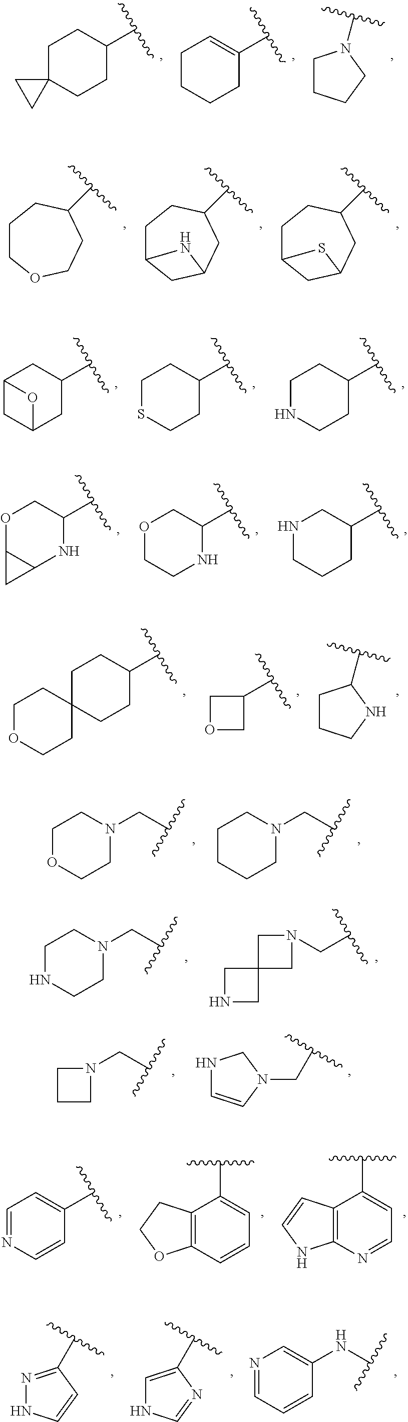

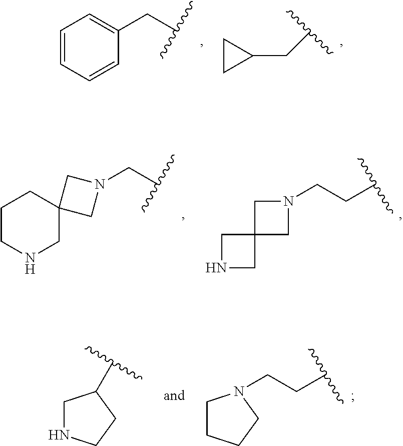 [SMILES: *Cc1ccccc1 ; *CC1CC1 ; *CN1CC2(CCCNC2)C1 ; *CCN1CC2(CNC2)C1 ; *C1CCNC1 ; *CCN1CCCC1]

the expression “optionally substituted” refers to the case of being unsubstituted or substituted with one or more of the substituents selected from: hydroxyl, cyano, F, Cl, Br, oxo, amido, —SO₂NH₂, methyl, ethyl, n-propyl, isopropyl, monofluoromethyl, difluoromethyl, trifluoromethyl, methoxy, hydroxymethyl, benzyl, phenyl optionally substituted with methyl or halogen, pyridyl optionally substituted with methyl or halogen, pyrazolyl optionally substituted with methyl or halogen, —SONHCH₃, —SO₂CH₃, —COCH₃, —COCH₂CH₃, —CO-cyclopropyl, —CO-cyclobutyl, —CO-cyclopentyl, —CO-phenyl, —NHSO₂CH₃ and —CONHCH₃;
further preferably, R^{A} is selected from hydrogen, carboxyl,

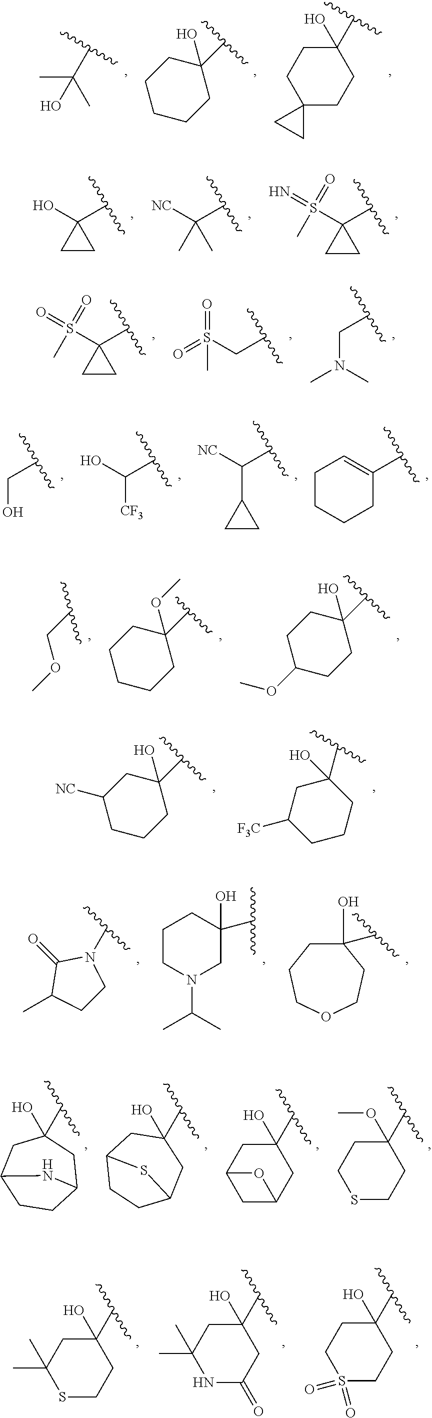

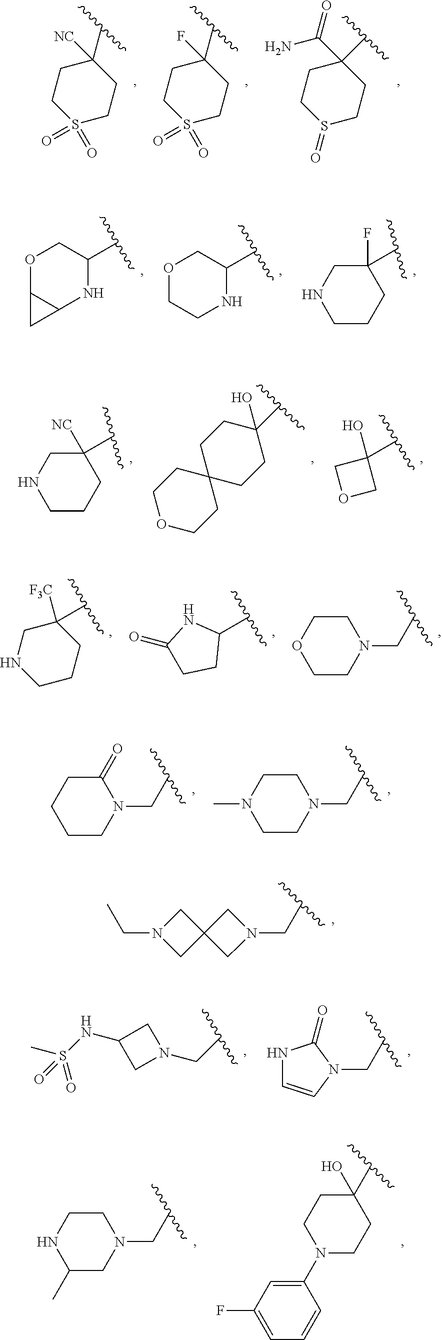

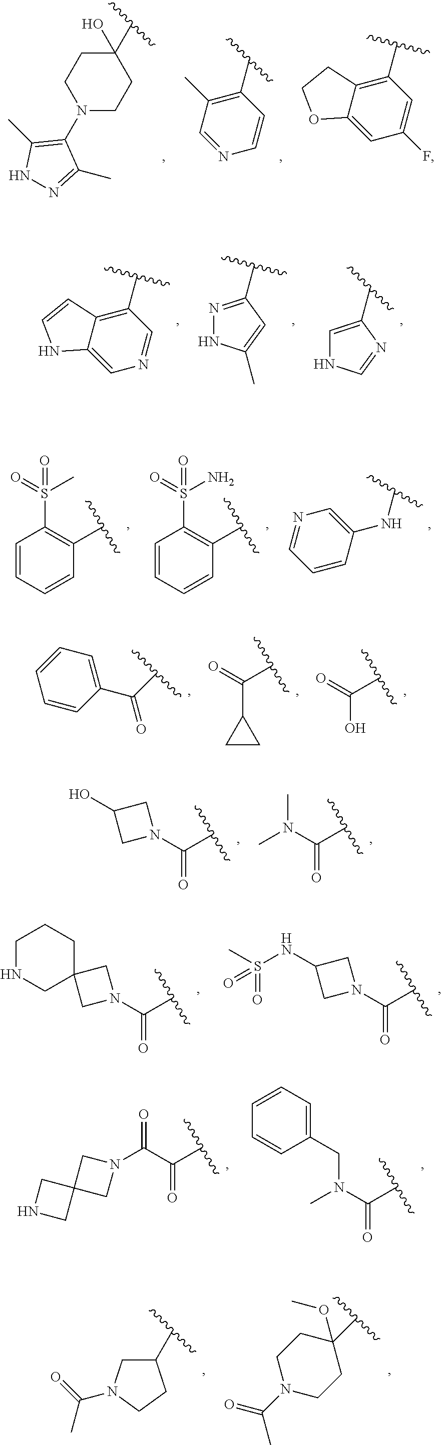

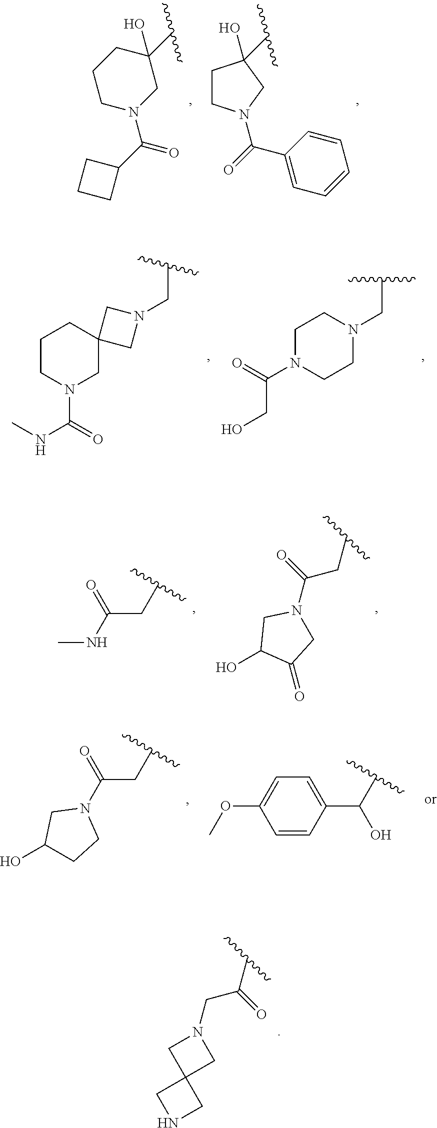

In a preferred embodiment of the present invention, the compound as shown in formula (V) is further represented by formula (VI):

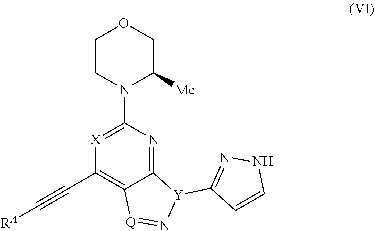

wherein the substituents in formula (VI) are as defined in formula (V).

The present invention further provides a compound as shown in formula (D), and a stereoisomer, an optical isomer, a pharmaceutical salt, a prodrug and a solvate thereof,

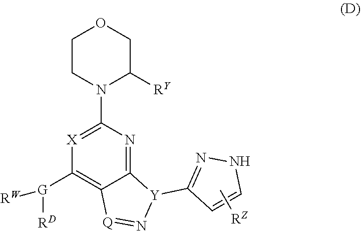

wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{Y} is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl; G is selected from a benzene ring or a pyridine ring;
the number of R^{W} is 1, 2 or 3, and each R^{W} is independently selected from hydrogen, halogen, cyano, amino, hydroxyl, carboxyl, sulfonyl, sulfonamido, sulfone, C₁₋₆ alkyl, halo C₁₋₆ alkyl, C₁₋₆ alkoxy, C₂₋₆ alkenyl, C₂₋₆ alkynyl, C₃₋₆ cycloalkyl, C₃₋₆ cycloalkenyl, 3- to 10-membered heterocyclyl, C₆₋₁₂ aryl, 5- to 10-membered heteroaryl, —NHC₁₋₆ alkyl or —N(C₁₋₆ alkyl)₂;
R^{D} is selected from —NR⁷C(O)R⁸ or —NR⁷C(O)NR⁷R⁸, wherein each R⁷ is independently selected from hydrogen, cyano, hydroxyl, halogen, C₁₋₃ alkyl, halo C₁₋₃ alkyl, C₃₋₆ carbocyclyl or aryl, and R⁸ is selected from the following substituent which is optionally substituted: C₁₋₆ alkyl, C₃₋₁₂ carbocyclyl, C₆₋₁₂ aryl, 3- to 12-membered heterocyclyl or 5- to 12-membered heteroaryl; the expression “optionally substituted” refers to the case of being unsubstituted or substituted with one or more of the following substituents: hydroxyl, amino, cyano, halogen, oxo, C₁₋₆ alkyl, halo C₁₋₆ alkyl, —S(O)₂C₁₋₆ alkyl and —COC₁₋₆ alkyl;
the heteroatoms in the “heterocyclyl” and “heteroaryl” are selected from N, O or S, and the number of the heteroatoms is 1, 2, 3 or 4.

In preferred embodiments of the present invention, Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio;
further preferably, Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio;
further preferably, Y is selected from N, and Q is selected from CR¹; R¹ is selected from hydrogen, halogen, cyano, amido, methyl, ethyl, methoxy, ethoxy, methylthio or ethylthio;
still further preferably, Y is selected from N, and Q is selected from CR¹; R¹ is selected from hydrogen, F, Cl, Br, cyano, amido, methyl, ethyl, methoxy or methylthio;
still further preferably, Y is selected from N, and Q is selected from CR¹; R¹ is selected from hydrogen, F, cyano, amido or methylthio;
most preferably, Y is selected from N, and Q is selected from CR¹; R¹ is selected from hydrogen.

In preferred embodiments of the present invention, X is selected from CR^{X}; wherein R^{X} is selected from hydrogen, halogen, hydroxyl, sulfhydryl, amino, cyano or C₁₋₆ alkyl; further preferably, R^{X} is selected from hydrogen, F, Cl, Br, hydroxyl, amino, cyano or methyl; still further preferably, R^{X} is selected from hydrogen.

In preferred embodiments of the present invention, R^{Y} is F, Cl, Br, methyl, ethyl, n-propyl, isopropyl or hydrogen;
further preferably, R^{Y} is F, Cl, Br, methyl, ethyl or hydrogen;
still further preferably, R^{Y} is methyl.

In preferred embodiments of the present invention, the number of R^{Z} is 0, 1, 2 or 3, and R^{Z}, at each occurrence, is independently selected from F, Cl, Br, hydroxyl, cyano, amino, methyl, ethyl, monofluoromethyl, difluoromethyl or trifluoromethyl;
further preferably, the number of R^{Z} is 0, 1 or 2, and R^{Z}, at each occurrence, is independently selected from F, Cl, Br, hydroxyl, methyl, monofluoromethyl, difluoromethyl or trifluoromethyl;
still further preferably, the number of R^{Z} is 0.

In preferred embodiments of the present invention, the number of R^{W} is 1, 2 or 3, and each R^{W} is independently selected from hydrogen, halogen, cyano, amino, hydroxyl, carboxyl, C₁₋₃ alkyl, halo C₁₋₃ alkyl, C₁₋₃ alkoxy, —NHC₁₋₃ alkyl or —N(C₁₋₃ alkyl)₂; further preferably, the number of R^{W} is 1, 2 or 3, and each R^{W} is independently selected from hydrogen, F, Cl, Br, cyano, amino, hydroxyl, carboxyl, methyl, ethyl, n-propyl, isopropyl, monofluoromethyl, difluoromethyl, trifluoromethyl, methoxy, —NHCH₃ or —N(CH₃)₂; further preferably, the number of R^{W} is 1, 2 or 3, and each R^{W} is independently selected from hydrogen, F, Cl, Br, cyano, amino, hydroxyl, carboxyl, methyl, monofluoromethyl, difluoromethyl, trifluoromethyl or methoxy;
still further preferably, the number of R^{W} is 1 or 2, and R^{W} is selected from hydrogen, methyl, F, cyano or methoxy.

In preferred embodiments of the present invention, R^{D} is selected from —NR⁷C(O)R⁸ or —NR⁷C(O)NR⁷R⁸, wherein each R⁷ is independently selected from hydrogen, cyano, hydroxyl, F, Cl, Br, methyl, ethyl, cyclopropyl or phenyl, and R⁸ is selected from the following substituent which is optionally substituted: C₁₋₄ alkyl, C₃₋₁₀ cycloalkyl, C₆₋₁₀ aryl, 3- to 8-membered heterocyclyl or 5-to 6-membered heteroaryl; the expression “optionally substituted” refers to the case of being unsubstituted or substituted with one or more of the following substituents: hydroxyl, amino, cyano, halogen, oxo, C₁₋₃ alkyl, halo C₁₋₃ alkyl, —S(O)₂C₁₋₃ alkyl and —COC₁₋₃ alkyl;
further preferably, R^{D} is selected from —NR⁷C(O)R⁸ or —NR⁷C(O)NR⁷R⁸, wherein each R⁷ is independently selected from hydrogen, cyano, methyl, ethyl, cyclopropyl or phenyl, and R⁸ is selected from the following substituent which is optionally substituted: methyl, ethyl, n-propyl, isopropyl, C₃₋₆ monocyclic cycloalkyl, phenyl, 3- to 6-membered monocyclic heterocyclyl, 7- to 9-membered bridged heterocyclyl, C₇₋₁₀ bridged cycloalkyl or 5- to 6-membered monocyclic heteroaryl; the expression “optionally substituted” refers to the case of being unsubstituted or substituted with one or more of the following substituents: hydroxyl, amino, cyano, halogen, oxo, C₁₋₃ alkyl, halo C₁₋₃ alkyl, —S(O)₂C₁₋₃ alkyl and —COC₁₋₃ alkyl;
further preferably, R^{D} is selected from —NR⁷C(O)R⁸ or —NR⁷C(O)NR⁷R⁸, wherein each R⁷ is independently selected from hydrogen, methyl, ethyl, cyclopropyl or phenyl, and R⁸ is selected from the following substituent which is optionally substituted: methyl, ethyl, n-propyl, isopropyl, cyclopropyl, cyclobutyl, cyclopentyl, cyclohexyl, phenyl, pyrrolidyl, tetrahydrofuryl, tetrahydropyranyl, piperidyl, pyridyl, thienyl, oxazolyl, thiazolyl, furyl, pyrazolyl, imidazolyl, pyrrolyl, piperazinyl, C₁₀ bridged cycloalkyl or 8-membered bridged heterocyclyl; the expression “optionally substituted” refers to the case of being unsubstituted or substituted with one or more of the following substituents: hydroxyl, amino, cyano, F, Cl, Br, oxo, methyl, ethyl, —S(O)₂CH₃alkyl and acetyl;
further preferably, G is a pyridine ring, and the pyridine ring connected to R^{D} and R^{W} is selected from

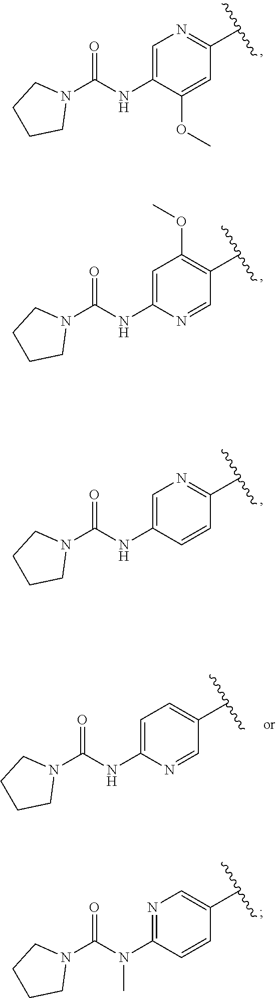 [SMILES: *c1cc(OC)c(NC(=O)N2CCCC2)cn1 ; *c1cnc(NC(=O)N2CCCC2)cc1OC ; *c1ccc(NC(=O)N2CCCC2)cn1 ; *c1ccc(NC(=O)N2CCCC2)nc1 ; *c1ccc(N(C)C(=O)N2CCCC2)nc1]

further preferably, G is a benzene ring, and the benzene ring connected to R^{D} and R^{W} is selected from the structure:

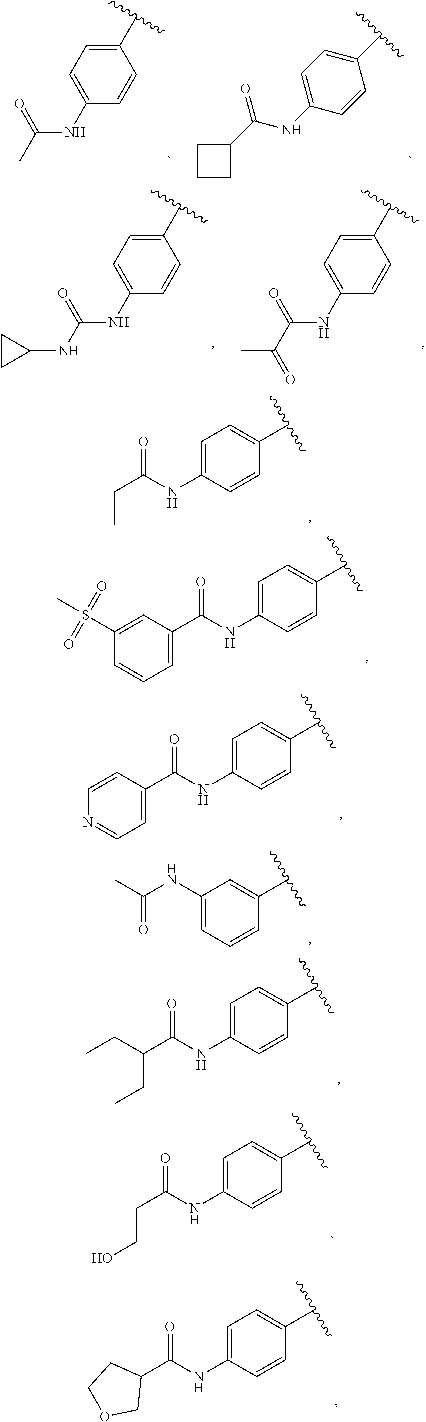

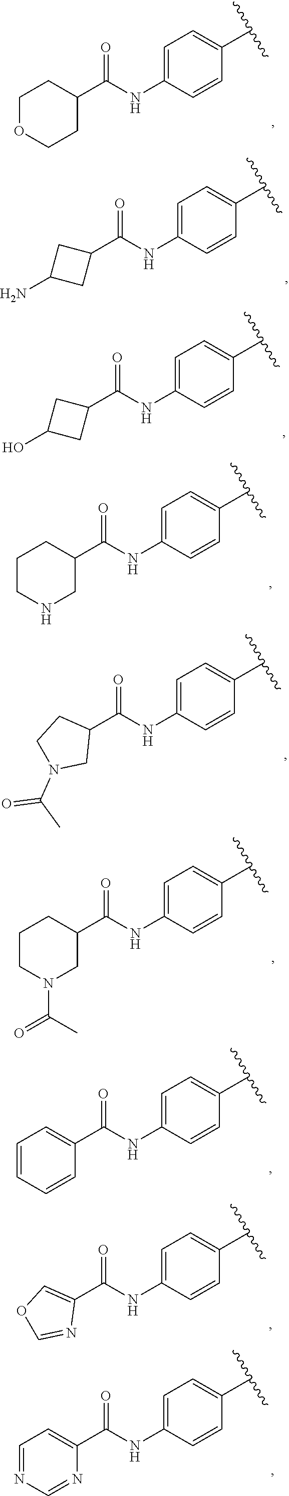

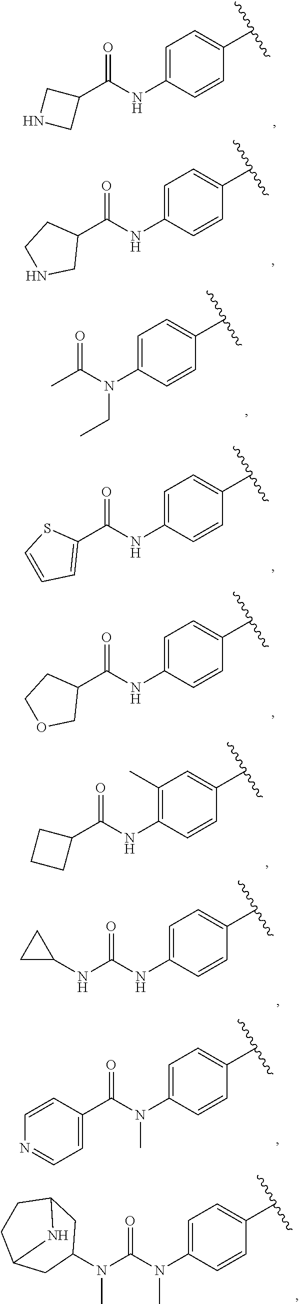

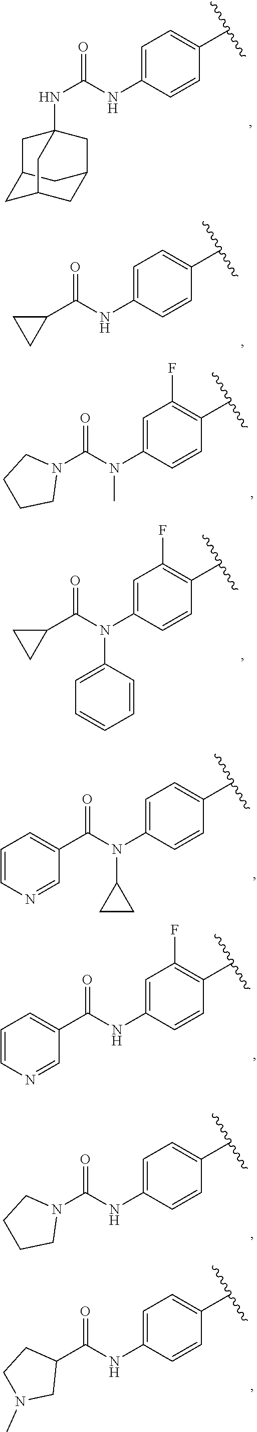

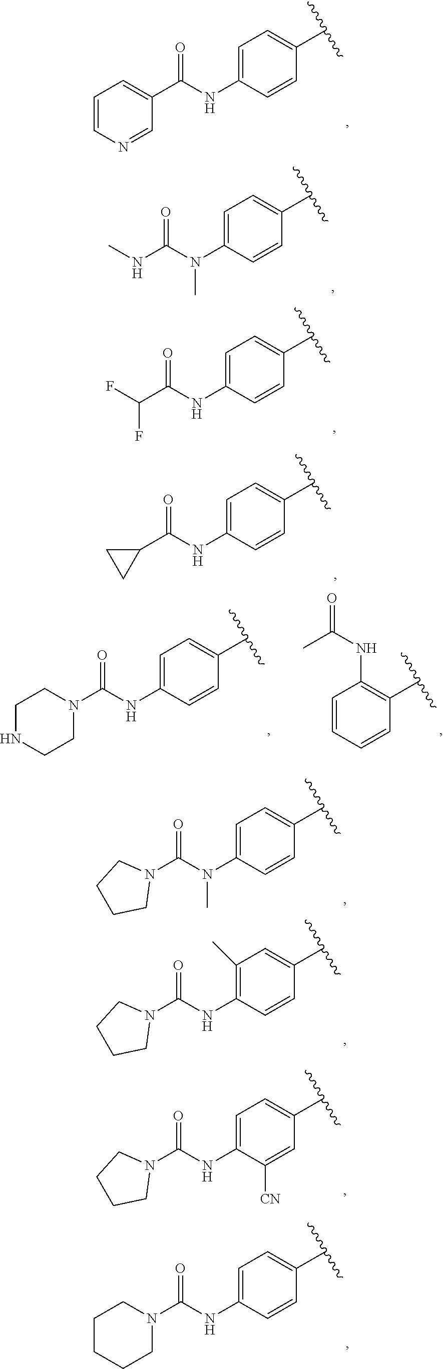

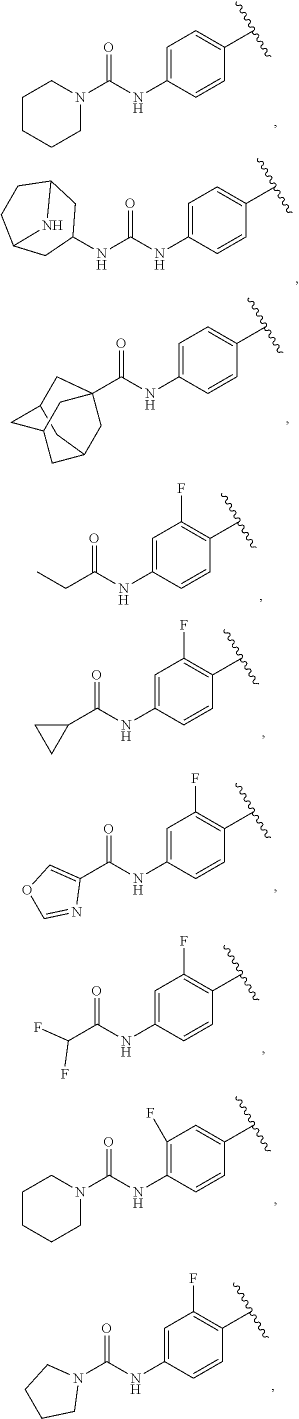

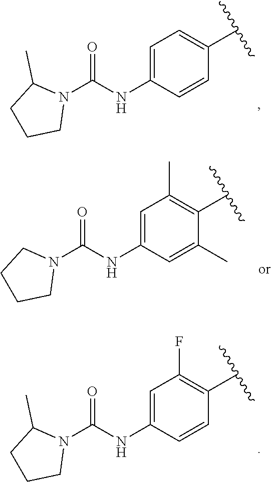 [SMILES: *c1ccc(NC(=O)N2CCCC2C)cc1 ; *c1c(C)cc(NC(=O)N2CCCC2)cc1C ; *c1ccc(NC(=O)N2CCCC2C)cc1F]

In a preferred embodiment of the present invention, the compound as shown in formula (D) is further represented by formula (D-1):

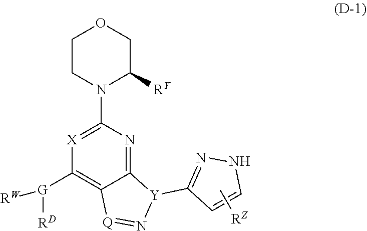

wherein the substituents in formula (D-1) are as defined in formula (D).

In a preferred embodiment of the present invention, the compound as shown in formula (D) is further represented by formula (D-2):

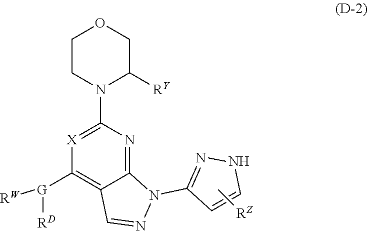

wherein the substituents in formula (D-2) are as defined in formula (D).

In a preferred embodiment of the present invention, the compound as shown in formula (D) is further represented by formula (D-3):

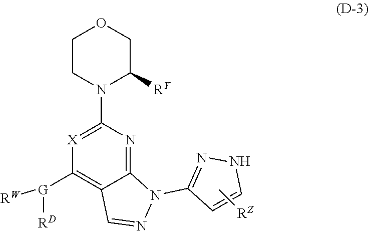

wherein the substituents in formula (D-3) are as defined in formula (D).

In a preferred embodiment of the present invention, the compound as shown in formula (D) is further represented by formula (D-4):

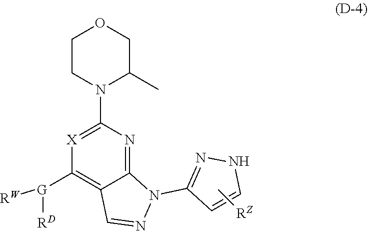

wherein the substituents in formula (D-4) are as defined in formula (D).

In a preferred embodiment of the present invention, the compound as shown in formula (D) is further represented by formula (D-5):

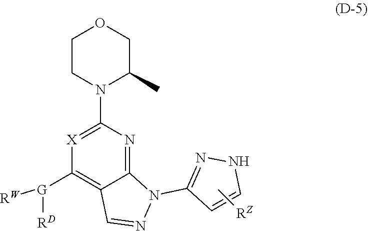

wherein the substituents in formula (D-5) are as defined in formula (D).

The present invention further provides a compound as shown in formula (VII), and a stereoisomer, an optical isomer, a pharmaceutical salt, a prodrug and a solvate thereof,

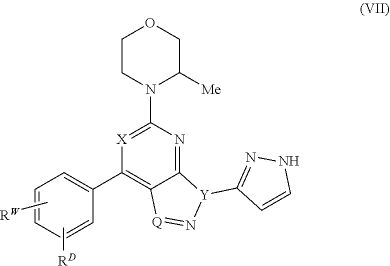

wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
the number of R^{W} is 1, 2 or 3, and each R^{W} is independently selected from hydrogen, halogen, cyano, amino, hydroxyl, carboxyl, sulfonyl, sulfonamido, sulfone, C₁₋₆ alkyl, halo C₁₋₆ alkyl, C₁₋₆ alkoxy, C₂₋₆ alkenyl, C₂₋₆ alkynyl, C₃₋₆ cycloalkyl, C₃₋₆ cycloalkenyl, 3- to 10-membered heterocyclyl, C₆₋₁₂ aryl, 5- to 10-membered heteroaryl, —NHC₁₋₆ alkyl or —N(C₁₋₆ alkyl)₂;
R^{D} is selected from —NR⁷C(O)R⁸ or —NR⁷C(O)NR⁷R⁸, wherein each R⁷ is independently selected from hydrogen, cyano, hydroxyl, halogen, C₁₋₃ alkyl, halo C₁₋₃ alkyl, C₃₋₆ carbocyclyl or aryl, and R⁸ is selected from the following substituent which is optionally substituted: C₁₋₆ alkyl, C₃₋₁₂ carbocyclyl, C₆₋₁₂ aryl, 3- to 12-membered heterocyclyl or 5- to 12-membered heteroaryl; the expression “optionally substituted” refers to the case of being unsubstituted or substituted with one or more of the following substituents: hydroxyl, amino, cyano, halogen, oxo, C₁₋₆ alkyl, halo C₁₋₆ alkyl, —S(O)₂C₁₋₆ alkyl and —COC₁₋₆ alkyl;
the heteroatoms in the “heterocyclyl” and “heteroaryl” are selected from N, O or S, and the number of the heteroatoms is 1, 2, 3 or 4.

In preferred embodiments of the present invention, Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio;
further preferably, Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio;
further preferably, Y is selected from N, and Q is selected from CR¹; R¹ is selected from hydrogen, halogen, cyano, amido, methyl, ethyl, methoxy, ethoxy, methylthio or ethylthio;
still further preferably, Y is selected from N, and Q is selected from CR¹; R¹ is selected from hydrogen, F, Cl, Br, cyano, amido, methyl, ethyl, methoxy or methylthio;
still further preferably, Y is selected from N, and Q is selected from CR¹; R¹ is selected from hydrogen, F, cyano, amido or methylthio.

In preferred embodiments of the present invention, X is selected from CR^{X}; wherein R^{X} is selected from hydrogen, halogen, hydroxyl, sulfhydryl, amino, cyano or C₁₋₆ alkyl;
further preferably, R^{X} is selected from hydrogen, F, Cl, Br, hydroxyl, amino, cyano or methyl;
still further preferably, R^{X} is selected from hydrogen.

In preferred embodiments of the present invention, the number of R^{W} is 1, 2 or 3, and each R^{W} is independently selected from hydrogen, halogen, cyano, amino, hydroxyl, carboxyl, C₁₋₃ alkyl, halo C₁₋₃ alkyl, C₁₋₃ alkoxy, —NHC₁₋₃ alkyl or —N(C₁₋₃ alkyl)₂;
further preferably, the number of R^{W} is 1, 2 or 3, and each R^{W} is independently selected from hydrogen, F, Cl, Br, cyano, amino, hydroxyl, carboxyl, methyl, ethyl, n-propyl, isopropyl, monofluoromethyl, difluoromethyl, trifluoromethyl, methoxy, —NHCH₃ or —N(CH₃)₂;
further preferably, the number of R^{W} is 1, 2 or 3, and each R^{W} is independently selected from hydrogen, F, Cl, Br, cyano, amino, hydroxyl, carboxyl, methyl, monofluoromethyl, difluoromethyl, trifluoromethyl or methoxy;
still further preferably, the number of R^{W} is 1 or 2, and R^{W} is selected from hydrogen, methyl or F.

In preferred embodiments of the present invention, R^{D} is selected from —NR⁷C(O)R⁸ or —NR⁷C(O)NR⁷R⁸, wherein each R⁷ is independently selected from hydrogen, cyano, hydroxyl, F, Cl, Br, methyl, ethyl, cyclopropyl or phenyl, and R⁸ is selected from the following substituent which is optionally substituted: C₁₋₄ alkyl, C₃₋₁₀ cycloalkyl, C₆₋₁₀ aryl, 3- to 8-membered heterocyclyl or 5-to 6-membered heteroaryl; the expression “optionally substituted” refers to the case of being unsubstituted or substituted with one or more of the following substituents: hydroxyl, amino, cyano, halogen, oxo, C₁₋₃ alkyl, halo C₁₋₃ alkyl, —S(O)₂C₁₋₃ alkyl and —COC₁₋₃ alkyl;
further preferably, R^{D} is selected from —NR⁷C(O)R⁸ or —NR⁷C(O)NR⁷R⁸, wherein each R⁷ is independently selected from hydrogen, cyano, methyl, ethyl, cyclopropyl or phenyl, and R⁸ is selected from the following substituent which is optionally substituted: methyl, ethyl, n-propyl, isopropyl, C₃₋₆ monocyclic cycloalkyl, phenyl, 3- to 6-membered monocyclic heterocyclyl, 7- to 9-membered bridged heterocyclyl, C₇₋₁₀ bridged cycloalkyl or 5- to 6-membered monocyclic heteroaryl; the expression “optionally substituted” refers to the case of being unsubstituted or substituted with one or more of the following substituents: hydroxyl, amino, cyano, halogen, oxo, C₁₋₃ alkyl, halo C₁₋₃ alkyl, —S(O)₂C₁₋₃ alkyl and —COC₁₋₃ alkyl;
further preferably, R^{D} is selected from —NR⁷C(O)R⁸ or —NR⁷C(O)NR⁷R⁸, wherein each R⁷ is independently selected from hydrogen, methyl, cyclopropyl or phenyl, and R⁸ is selected from the following substituent which is optionally substituted: methyl, ethyl, n-propyl, isopropyl, cyclopropyl, cyclobutyl, cyclopentyl, cyclohexyl, phenyl, pyrrolidyl, tetrahydrofuryl, tetrahydropyranyl, piperidyl, pyridyl, thienyl, oxazolyl, thiazolyl, furyl, pyrazolyl, imidazolyl, pyrrolyl, C₁₀ bridged cycloalkyl or 8-membered bridged heterocyclyl; the expression “optionally substituted” refers to the case of being unsubstituted or substituted with one or more of the following substituents: hydroxyl, amino, cyano, F, Cl, Br, oxo, methyl, ethyl, —S(O)₂CH₃alkyl and acetyl;
further preferably, R^{D} is para to a fused ring connected to a benzene ring;
still further preferably, the benzene ring connected to R^{D} and R^{W} is selected from the structure:

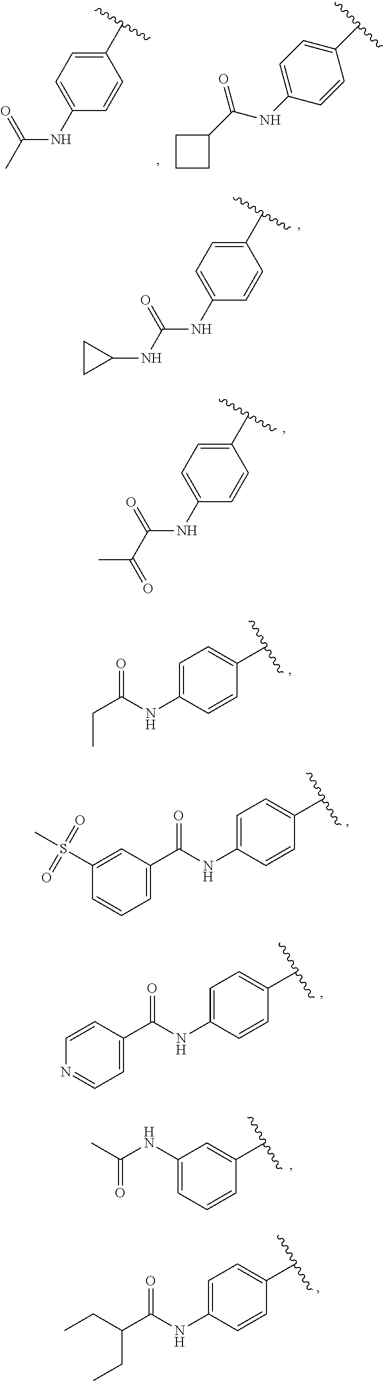

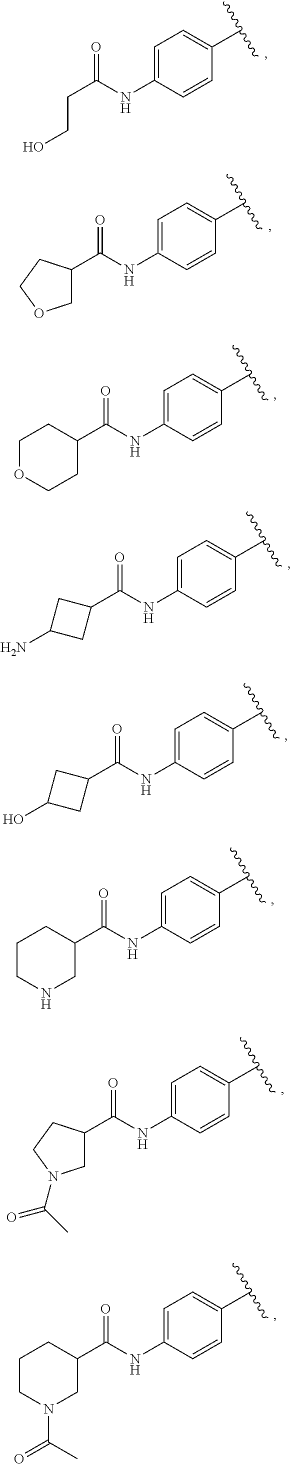

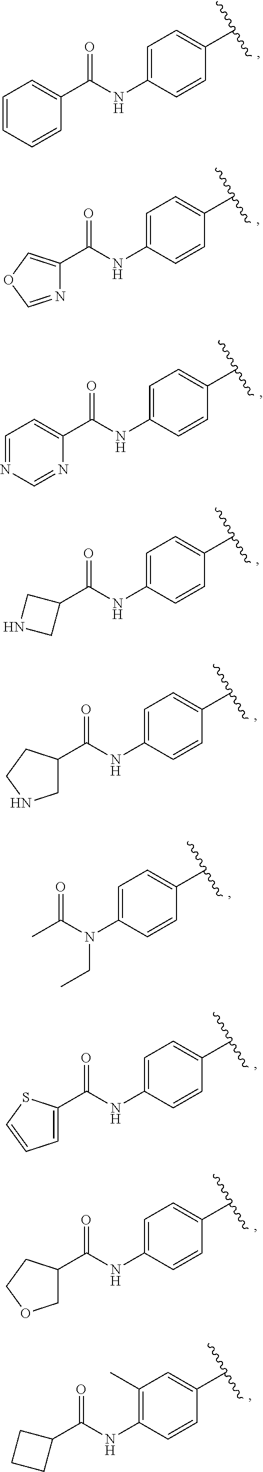

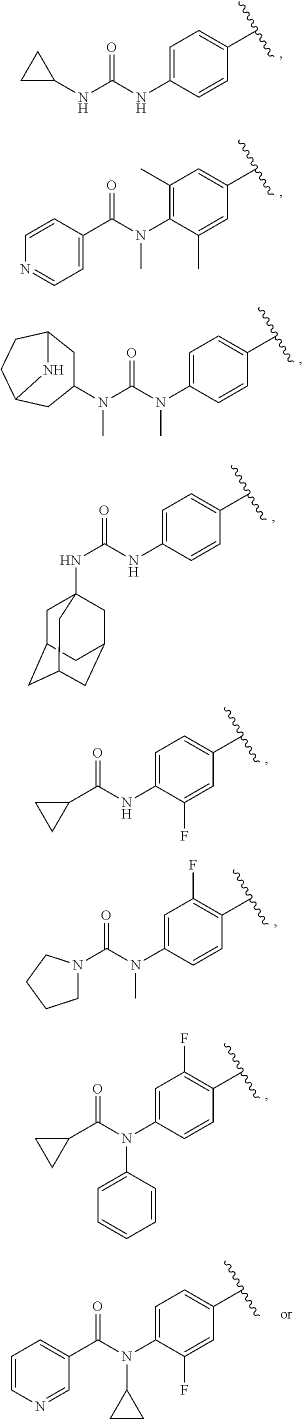

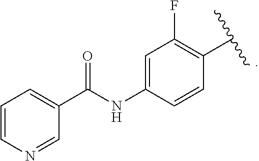 [SMILES: *c1ccc(NC(=O)c2cccnc2)cc1F]

In a preferred embodiment of the present invention, the compound as shown in formula (VII) is further represented by formula (IX):

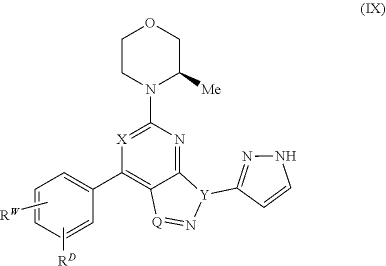

wherein the substituents in formula (IX) are as defined in formula (VII).

The present invention further provides a compound as shown in formula (E), and a stereoisomer, an optical isomer, a pharmaceutical salt, a prodrug and a solvate thereof,

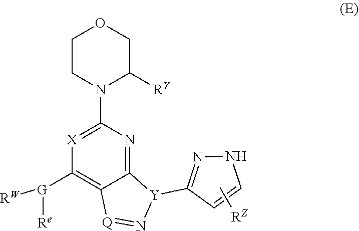

wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{Y} is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl;
G is selected from a benzene ring or a pyridine ring;
the number of R^{W} is 1, 2 or 3, and each R^{W} is independently selected from hydrogen, halogen, cyano, amino, hydroxyl, carboxyl, sulfonyl, sulfonamido, sulfone, C₁₋₆ alkyl, halo C₁₋₆ alkyl, C₁₋₆ alkoxy, C₂₋₆ alkenyl, C₂₋₆ alkynyl, C₃₋₆ cycloalkyl, C₃₋₆ cycloalkenyl, 3- to 10-membered heterocyclyl, C₆₋₁₂ aryl, 5- to 10-membered heteroaryl, —NHC₁₋₆ alkyl or —N(C₁₋₆ alkyl)₂;
R^{e} is selected from 4- to 12-membered heterocyclyl, 5- to 12-membered heteroaryl, C₃₋₁₂ carbocyclyl or C₆₋₁₂ aryl, wherein the 4- to 12-membered heterocyclyl, 5- to 12-membered heteroaryl, C₃₋₁₂ carbocyclyl and C₆₋₁₂ aryl are optionally substituted with one or more of the following substituents: halogen, hydroxyl, amino, cyano, nitro, carboxyl, oxo, C₁₋₆ alkyl, halo C₁₋₆ alkyl, C₁₋₆ alkoxy, C₁₋₆ hydroxyalkyl, C₂₋₆ alkenyl, C₂₋₆ alkynyl, C₃₋₆ cycloalkyl, C₃₋₆ cycloalkenyl, 3- to 10-membered heterocyclyl, —NHC₁₋₆ alkyl, —N(C₁₋₆ alkyl)₂, —C₁₋₆ alkyl-O—C₁₋₆ alkyl and —C₁₋₆ alkyl-C₆₋₁₂ aryl;
the heteroatoms in the “heterocyclyl” and “heteroaryl” are selected from N, O or S, and the number of the heteroatoms is 1, 2, 3 or 4.

In preferred embodiments of the present invention, Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio;
further preferably, Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio;
further preferably, Y is selected from N, and Q is selected from CR¹; R¹ is selected from hydrogen, halogen, cyano, amido, methyl, ethyl, methoxy, ethoxy, methylthio or ethylthio;
still further preferably, Y is selected from N, and Q is selected from CR¹; R¹ is selected from hydrogen, F, Cl, Br, cyano, amido, methyl, ethyl, methoxy or methylthio;
still further preferably, Y is selected from N, and Q is selected from CR¹; R¹ is selected from hydrogen, F, cyano, amido or methylthio;
most preferably, Y is selected from N, and Q is selected from CR¹; R¹ is selected from hydrogen.

In preferred embodiments of the present invention, X is selected from CR^{X}; wherein R^{X} is selected from hydrogen, halogen, hydroxyl, sulfhydryl, amino, cyano or C₁₋₆ alkyl;
further preferably, R^{X} is selected from hydrogen, F, Cl, Br, hydroxyl, amino, cyano or methyl;
still further preferably, R^{X} is selected from hydrogen.

In preferred embodiments of the present invention, R^{Y} is F, Cl, Br, methyl, ethyl, n-propyl, isopropyl or hydrogen;
further preferably, R^{Y} is F, Cl, Br, methyl, ethyl or hydrogen;
still further preferably, R^{Y} is methyl.

In preferred embodiments of the present invention, the number of R^{Z} is 0, 1, 2 or 3, and R^{Z}, at each occurrence, is independently selected from F, Cl, Br, hydroxyl, cyano, amino, methyl, ethyl, monofluoromethyl, difluoromethyl or trifluoromethyl;
further preferably, the number of R^{Z} is 0, 1 or 2, and R^{Z}, at each occurrence, is independently selected from F, Cl, Br, hydroxyl, methyl, monofluoromethyl, difluoromethyl or trifluoromethyl;
still further preferably, the number of R^{Z} is 0.

In preferred embodiments of the present invention, the number of R^{W} is 1, 2 or 3, and each R^{W} is independently selected from hydrogen, halogen, cyano, amino, hydroxyl, carboxyl, C₁-3 alkyl, halo C₁₋₃ alkyl, C₁₋₃ alkoxy, —NHC₁₋₃ alkyl or —N(C₁₋₃ alkyl)₂;

further preferably, the number of R^{W} is 1, 2 or 3, and each R^{W} is independently selected from hydrogen, F, Cl, Br, cyano, amino, hydroxyl, carboxyl, methyl, ethyl, n-propyl, isopropyl, monofluoromethyl, difluoromethyl, trifluoromethyl, methoxy, —NHCH₃ or —N(CH₃)₂;
further preferably, the number of R^{W} is 1, 2 or 3, and each R^{W} is independently selected from hydrogen, F, Cl, Br, cyano, amino, hydroxyl, carboxyl, methyl, monofluoromethyl, difluoromethyl, trifluoromethyl, methoxy or —NHCH₃;
still further preferably, the number of R^{W} is 1 or 2, and each R^{W} is independently selected from hydrogen, methyl, cyano, F, trifluoromethyl or —NHCH₃.

In preferred embodiments of the present invention, R^{e} is selected from 4- to 7-membered monocyclic heterocyclyl, 6- to 8-membered bridged heterocyclyl, 7- to 11-membered spiro heterocyclyl, 6- to 10-membered fused heterocyclyl, 5- to 6-membered monocyclic heteroaryl, C₅₋₆ monocyclic cycloalkyl, C₆ cycloalkenyl or phenyl, wherein the 4- to 7-membered monocyclic heterocyclyl, 6- to 8-membered bridged heterocyclyl, 7- to 11-membered spiro heterocyclyl, 8- to 10-membered fused heterocyclyl, 5- to 6-membered monocyclic heteroaryl, C₃₋₆ monocyclic cycloalkyl, C₆ cycloalkenyl and phenyl are optionally substituted with one or more of the following substituents: halogen, hydroxyl, amino, cyano, nitro, carboxyl, oxo, C₁₋₃ alkyl, halo C₁₋₃ alkyl, C₁₋₃ alkoxy, C₁₋₃ hydroxyalkyl, C₃₋₆ cycloalkyl, 3- to 6-membered monocyclic heterocyclyl, —NHC₁₋₃ alkyl, —N(C₁₋₃ alkyl)₂, C₁₋₃ alkyl-O—C₁₋₃ alkyl and —C₁₋₃ alkyl-phenyl; further preferably, R^{e} is selected from the following substituent which is optionally substituted: 4-membered monocyclic heterocyclyl, 5-membered monocyclic heterocyclyl, 6-membered monocyclic heterocyclyl, 7-membered monocyclic heterocyclyl, 7-membered bridged heterocyclyl, 8-membered bridged heterocyclyl, 4-membered/4-membered spiro heterocyclyl, 4-membered/5-membered spiro heterocyclyl, 5-membered/4-membered spiro heterocyclyl, 5-membered/5-membered spiro heterocyclyl, 4-membered/6-membered spiro heterocyclyl, 6-membered/4-membered spiro heterocyclyl, 5-membered/6-membered spiro heterocyclyl, 6-membered/5-membered spiro heterocyclyl, 6-membered/6-membered spiro heterocyclyl, 5-membered/3-membered fused heterocyclyl, 5-membered/5-membered fused heterocyclyl, 5-membered/6-membered fused heterocyclyl, 6-membered/5-membered fused heterocyclyl, 6-membered/6-membered fused heterocyclyl, 5-membered monocyclic heteroaryl, 6-membered monocyclic heteroaryl, cyclopentyl, cyclohexyl, C₆ cycloalkenyl or phenyl; the expression “optionally substituted” refers to the case of being unsubstituted or substituted with one or more of the following substituents: halogen, hydroxyl, amino, cyano, nitro, carboxyl, oxo, methyl, ethyl, n-propyl, isopropyl, monofluoromethyl, difluoromethyl, trifluoromethyl, methoxy, ethoxy, hydroxymethyl, hydroxyethyl, cyclopropyl, cyclobutyl, 3-membered monocyclic heterocyclyl, 4-membered monocyclic heterocyclyl, —CH₂OCH₃, —CH₂CH₂OCH₃ and —CH₂-phenyl; further preferably, R^{e} is selected from the following substituent which is optionally substituted:

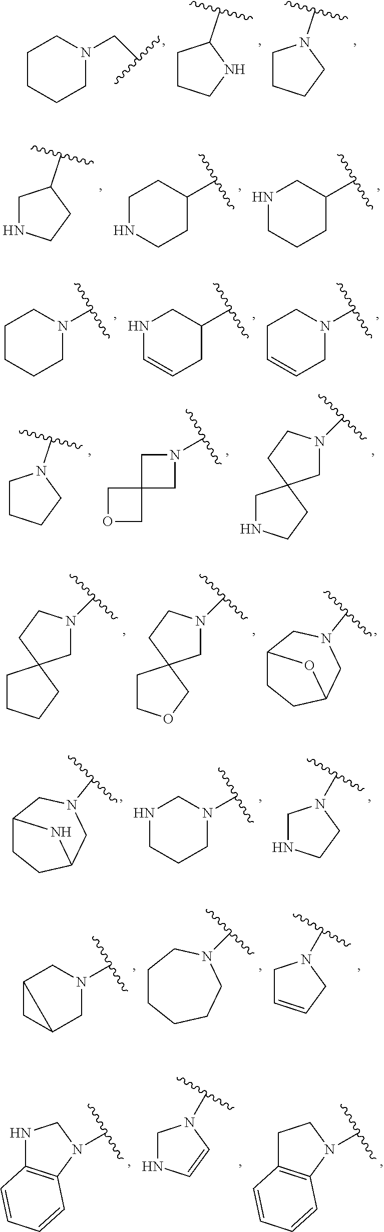

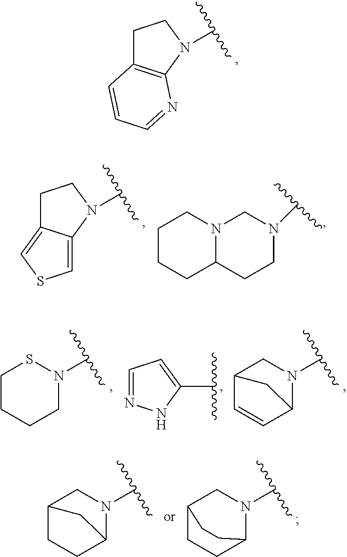

the expression “optionally substituted” refers to the case of being unsubstituted or substituted with one or more of the following substituents: F, Cl, Br, hydroxyl, cyano, oxo, methyl, ethyl, n-propyl, isopropyl, hydroxymethyl, hydroxyethyl, cyclopropyl, cyclobutyl, —CH₂OCH₃, —CH₂CH₂OCH₃ and —CH₂-phenyl;
still further preferably, G is a pyridine ring, and the pyridine ring connected to R^{W} and R^{e} is selected from the structure:

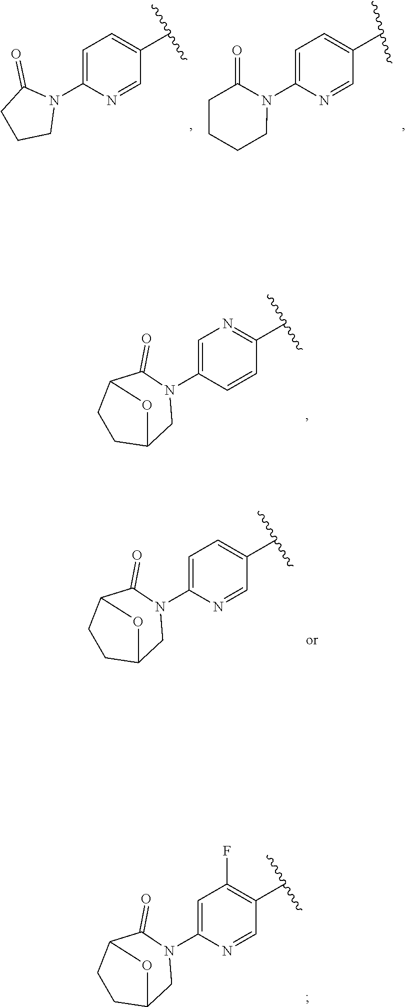 [SMILES: *c1ccc(N2CCCC2=O)nc1 ; *c1ccc(N2CCCCC2=O)nc1 ; *c1ccc(N2CC3CCC(O3)C2=O)cn1 ; *c1ccc(N2CC3CCC(O3)C2=O)nc1 ; *c1cnc(N2CC3CCC(O3)C2=O)cc1F]

still further preferably, G is a benzene ring, and the benzene ring connected to R^{W} and R^{e} is selected from the structure:

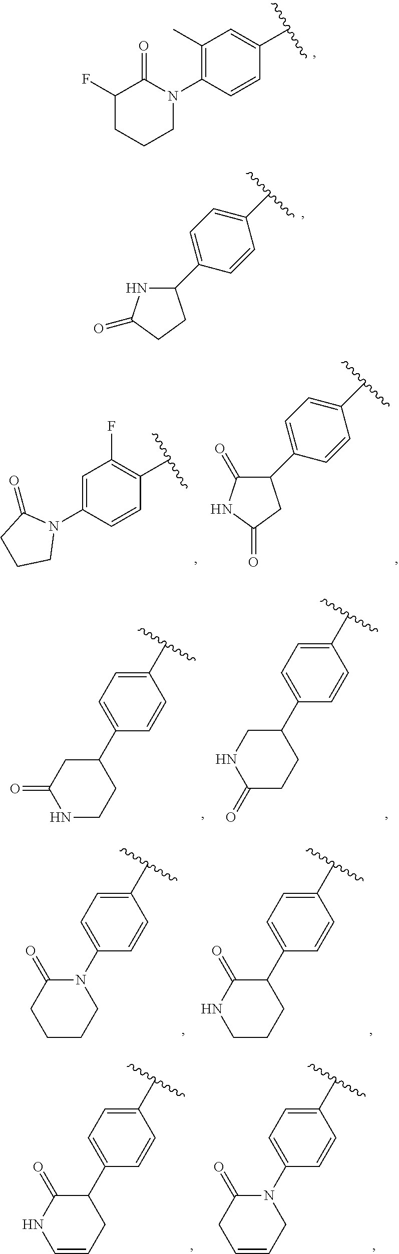

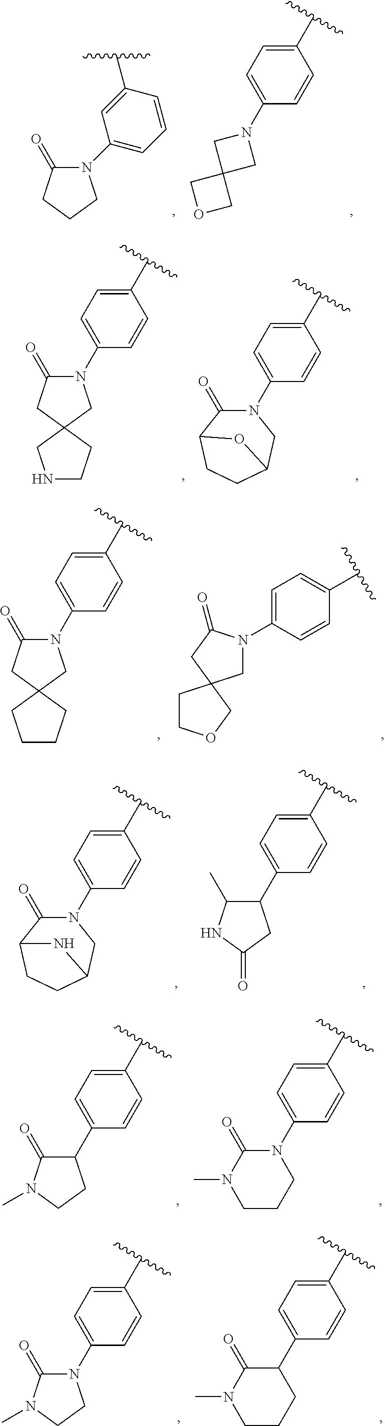

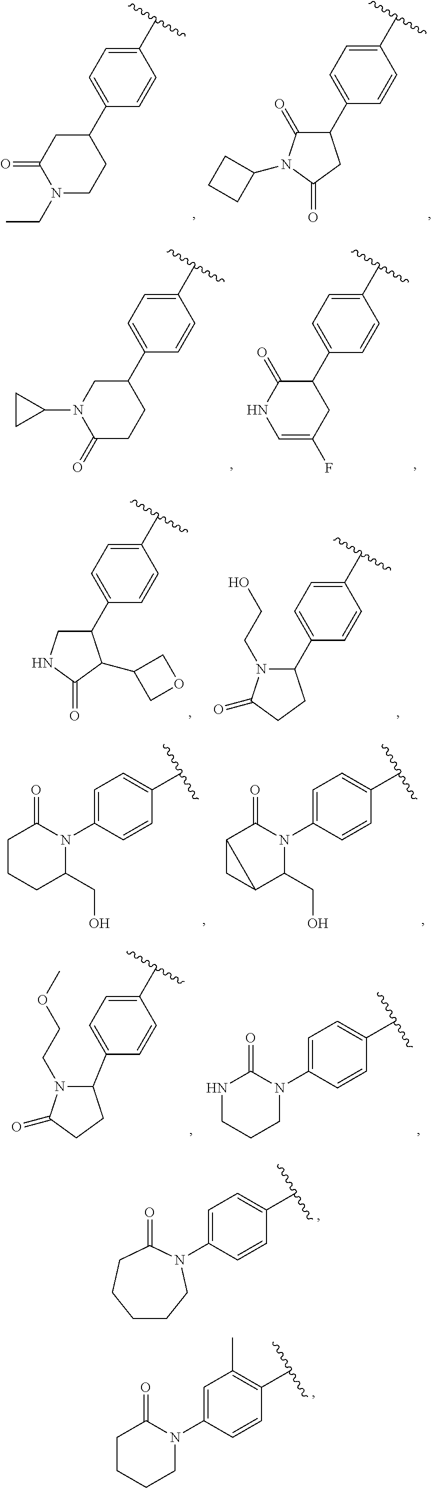

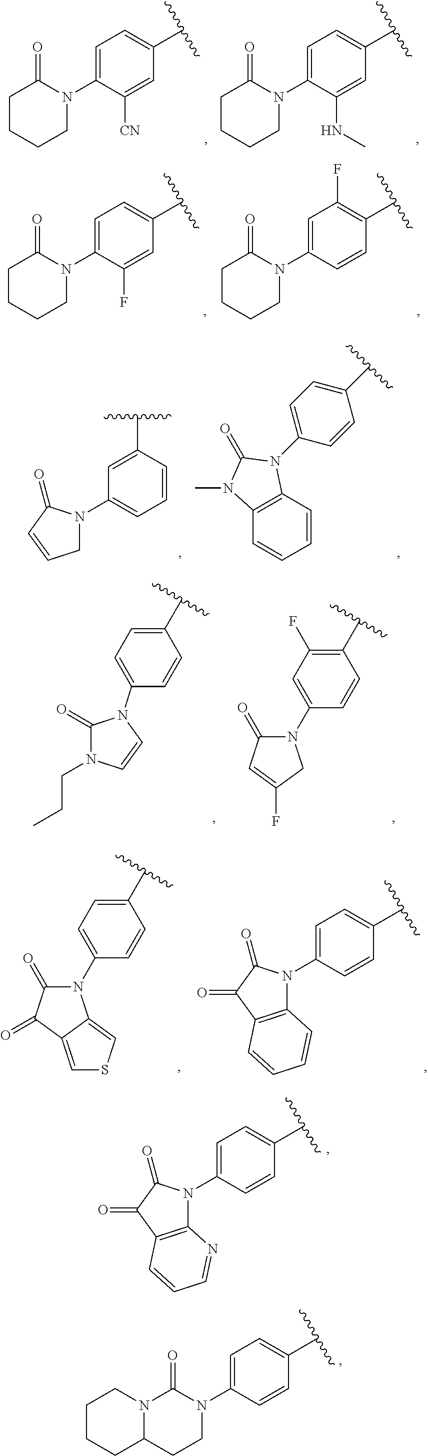

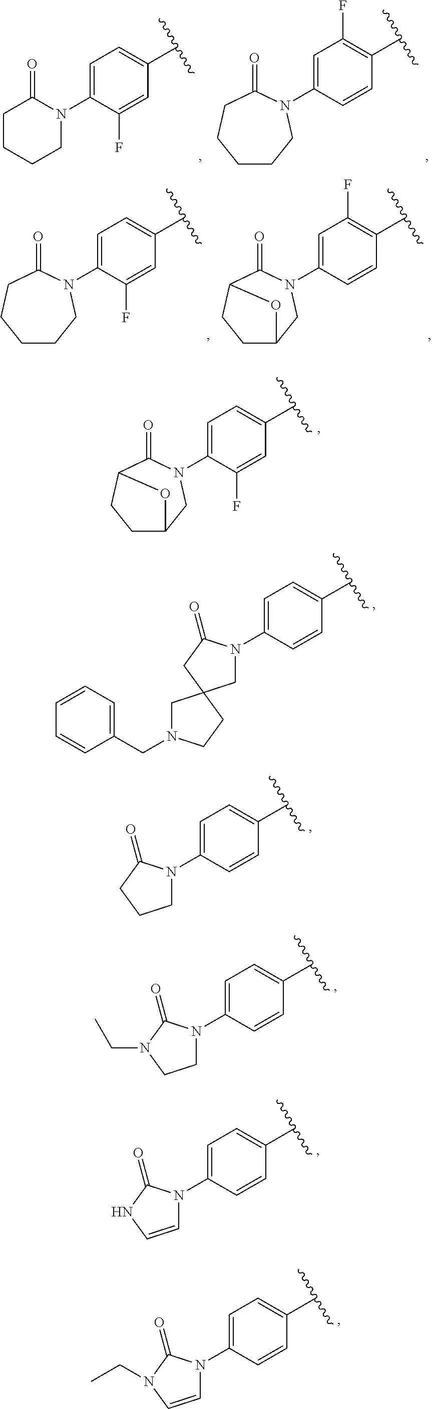

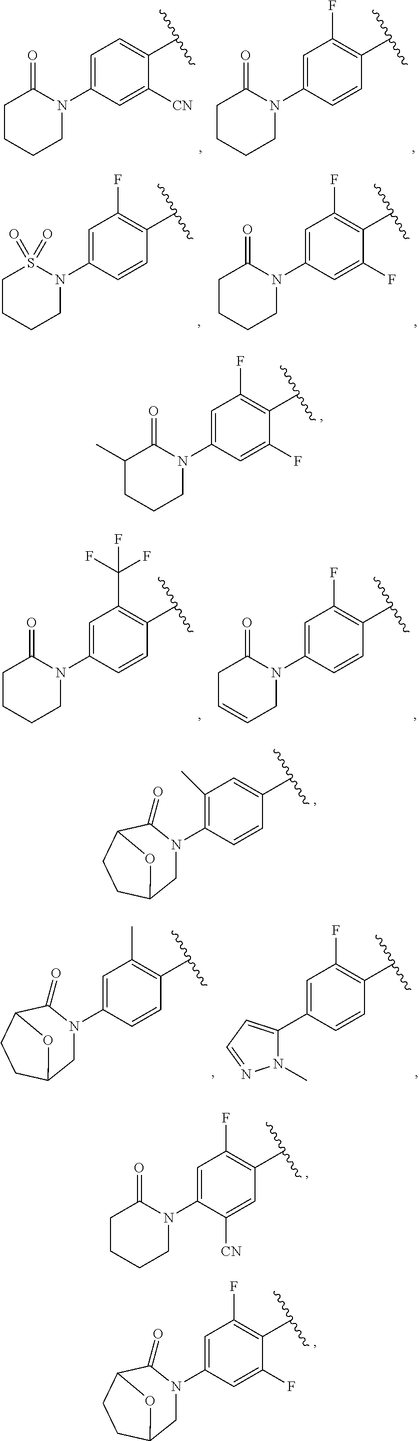

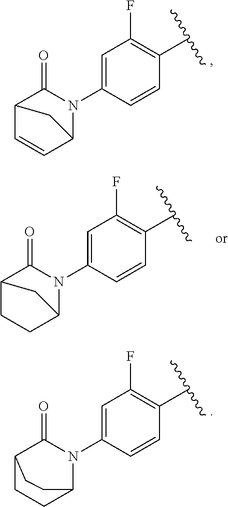 [SMILES: *c1ccc(N2C(=O)C3C=CC2C3)cc1F ; *c1ccc(N2C(=O)C3CCC2C3)cc1F ; *c1ccc(N2C(=O)C3CCC2CC3)cc1F]

In a preferred embodiment of the present invention, the compound as shown in formula (E) is further represented by formula (E-1):

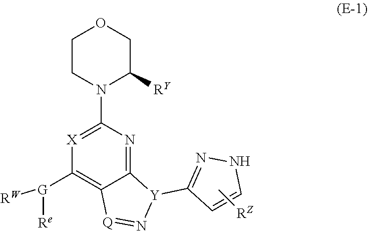

wherein the substituents in formula (E-1) are as defined in formula (E).

In a preferred embodiment of the present invention, the compound as shown in formula (E) is further represented by formula (E-2):

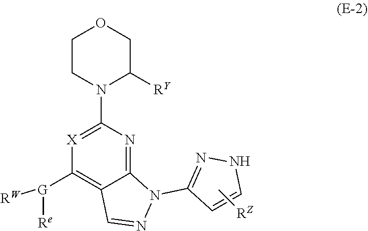

wherein the substituents in formula (E-2) are as defined in formula (E).

In a preferred embodiment of the present invention, the compound as shown in formula (E) is further represented by formula (E-3):

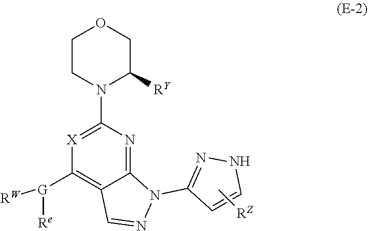

wherein the substituents in formula (E-3) are as defined in formula (E).

In a preferred embodiment of the present invention, the compound as shown in formula (E) is further represented by formula (E-4):

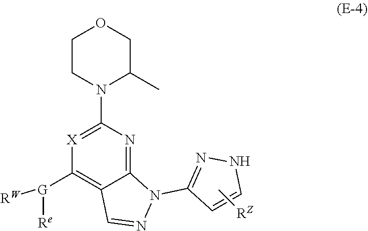

wherein the substituents in formula (E-4) are as defined in formula (E).

In a preferred embodiment of the present invention, the compound as shown in formula (E) is further represented by formula (E-5):

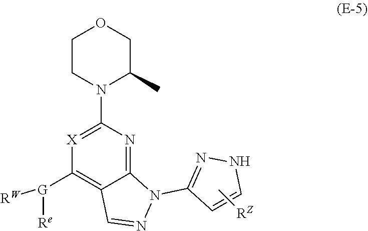

wherein the substituents in formula (E-5) are as defined in formula (E).

The present invention further provides a compound as shown in formula (X), and a stereoisomer, an optical isomer, a pharmaceutical salt, a prodrug and a solvate thereof,

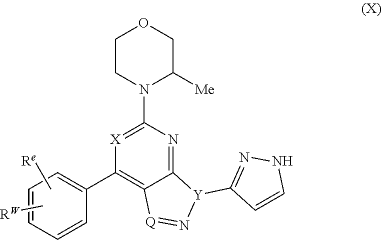

wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
the number of R^{W} is 1, 2 or 3, and each R^{W} is independently selected from hydrogen, halogen, cyano, amino, hydroxyl, carboxyl, sulfonyl, sulfonamido, sulfone, C₁₋₆ alkyl, halo C₁₋₆ alkyl, C₁₋₆ alkoxy, C₂₋₆ alkenyl, C₂₋₆ alkynyl, C₃₋₆ cycloalkyl, C₃₋₆ cycloalkenyl, 3- to 10-membered heterocyclyl, C₆₋₁₂ aryl, 5- to 10-membered heteroaryl, —NHC₁₋₆ alkyl or —N(C₁₋₆ alkyl)₂;
R^{e} is selected from 4- to 12-membered heterocyclyl, 5- to 12-membered heteroaryl, C₃₋₁₂ carbocyclyl or C₆₋₁₂ aryl, wherein the 4- to 12-membered heterocyclyl, 5- to 12-membered heteroaryl, C₃₋₁₂ carbocyclyl and C₆₋₁₂ aryl are optionally substituted with one or more of the following substituents: halogen, hydroxyl, amino, cyano, nitro, carboxyl, oxo, C₁₋₆ alkyl, halo C₁₋₆ alkyl, C₁₋₆ alkoxy, C₁₋₆ hydroxyalkyl, C₂₋₆ alkenyl, C₂₋₆ alkynyl, C₃₋₆ cycloalkyl, C₃₋₆ cycloalkenyl, 3- to 10-membered heterocyclyl, —NHC₁₋₆ alkyl, —N(C₁₋₆ alkyl)₂ and C₁₋₆ alkyl-O—C₁₋₆ alkyl; the heteroatoms in the “heterocyclyl” and “heteroaryl” are selected from N, O or S, and the number of the heteroatoms is 1, 2, 3 or 4.

In preferred embodiments of the present invention, Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio;
further preferably, Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio;
further preferably, Y is selected from N, and Q is selected from CR¹; R¹ is selected from hydrogen, halogen, cyano, amido, methyl, ethyl, methoxy, ethoxy, methylthio or ethylthio;
still further preferably, Y is selected from N, and Q is selected from CR¹; R¹ is selected from hydrogen, F, Cl, Br, cyano, amido, methyl, ethyl, methoxy or methylthio;
still further preferably, Y is selected from N, and Q is selected from CR¹; R¹ is selected from hydrogen, F, cyano, amido or methylthio.

In preferred embodiments of the present invention, X is selected from CR^{X}; wherein R^{X} is selected from hydrogen, halogen, hydroxyl, sulfhydryl, amino, cyano or C₁₋₆ alkyl;
further preferably, R^{X} is selected from hydrogen, F, Cl, Br, hydroxyl, amino, cyano or methyl;
still further preferably, R^{X} is selected from hydrogen.

In preferred embodiments of the present invention, the number of R^{W} is 1, 2 or 3, and each R^{W} is independently selected from hydrogen, halogen, cyano, amino, hydroxyl, carboxyl, C₁-3 alkyl, halo C₁₋₃ alkyl, C₁₋₃ alkoxy, —NHC₁₋₃ alkyl or —N(C₁₋₃ alkyl)₂;
further preferably, the number of R^{W} is 1, 2 or 3, and each R^{W} is independently selected from hydrogen, F, Cl, Br, cyano, amino, hydroxyl, carboxyl, methyl, ethyl, n-propyl, isopropyl, monofluoromethyl, difluoromethyl, trifluoromethyl, methoxy, —NHCH₃ or —N(CH₃)₂;
further preferably, the number of R^{W} is 1, 2 or 3, and each R^{W} is independently selected from hydrogen, F, Cl, Br, cyano, amino, hydroxyl, carboxyl, methyl, monofluoromethyl, difluoromethyl, trifluoromethyl, methoxy or —NHCH₃;
still further preferably, the number of R^{W} is 1, and R^{W} is selected from hydrogen, methyl, cyano, F or —NHCH₃.

In preferred embodiments of the present invention, R^{e} is selected from 4- to 7-membered monocyclic heterocyclyl, 6- to 8-membered bridged heterocyclyl, 7- to 11-membered spiro heterocyclyl, 6- to 10-membered fused heterocyclyl, 5- to 6-membered monocyclic heteroaryl, C₃₋₆ monocyclic cycloalkyl, C₆ cycloalkenyl or phenyl, wherein the 4- to 7-membered monocyclic heterocyclyl, 6- to 8-membered bridged heterocyclyl, 7- to 11-membered spiro heterocyclyl, 8- to 10-membered fused heterocyclyl, 5- to 6-membered monocyclic heteroaryl, C₃₋₆ monocyclic cycloalkyl, C₆ cycloalkenyl and phenyl are optionally substituted with one or more of the following substituents: halogen, hydroxyl, amino, cyano, nitro, carboxyl, oxo, C₁₋₃ alkyl, halo C₁₋₃ alkyl, C₁₋₃ alkoxy, C₁₋₃ hydroxyalkyl, C₃₋₆ cycloalkyl, 3- to 6-membered monocyclic heterocyclyl, —NHC₁₋₃ alkyl, —N(C₁₋₃ alkyl)₂ and C₁₋₃ alkyl-O—C₁₋₃ alkyl;
further preferably, R^{e} is selected from the following substituent which is optionally substituted: 4-membered monocyclic heterocyclyl, 5-membered monocyclic heterocyclyl, 6-membered monocyclic heterocyclyl, 7-membered monocyclic heterocyclyl, 7-membered bridged heterocyclyl, 8-membered bridged heterocyclyl, 4-membered/4-membered spiro heterocyclyl, 4-membered/5-membered spiro heterocyclyl, 5-membered/4-membered spiro heterocyclyl, 5-membered/5-membered spiro heterocyclyl, 4-membered/6-membered spiro heterocyclyl, 6-membered/4-membered spiro heterocyclyl, 5-membered/6-membered spiro heterocyclyl, 6-membered/5-membered spiro heterocyclyl, 6-membered/6-membered spiro heterocyclyl, 5-membered/3-membered fused heterocyclyl, 5-membered/5-membered fused heterocyclyl, 5-membered/6-membered fused heterocyclyl, 6-membered/5-membered fused heterocyclyl, 6-membered/6-membered fused heterocyclyl, 5-membered monocyclic heteroaryl, 6-membered monocyclic heteroaryl, cyclopropyl, cyclobutyl, cyclopentyl, cyclohexyl, C₆ cycloalkenyl or phenyl; the expression “optionally substituted” refers to the case of being unsubstituted or substituted with one or more of the following substituents: halogen, hydroxyl, amino, cyano, nitro, carboxyl, oxo, methyl, ethyl, n-propyl, isopropyl, monofluoromethyl, difluoromethyl, trifluoromethyl, methoxy, ethoxy, hydroxymethyl, hydroxyethyl, cyclopropyl, cyclobutyl, 3-membered monocyclic heterocyclyl, 4-membered monocyclic heterocyclyl, —CH₂OCH₃ and —CH₂CH₂OCH₃;
further preferably, R^{e} is selected from the following substituent which is optionally substituted:

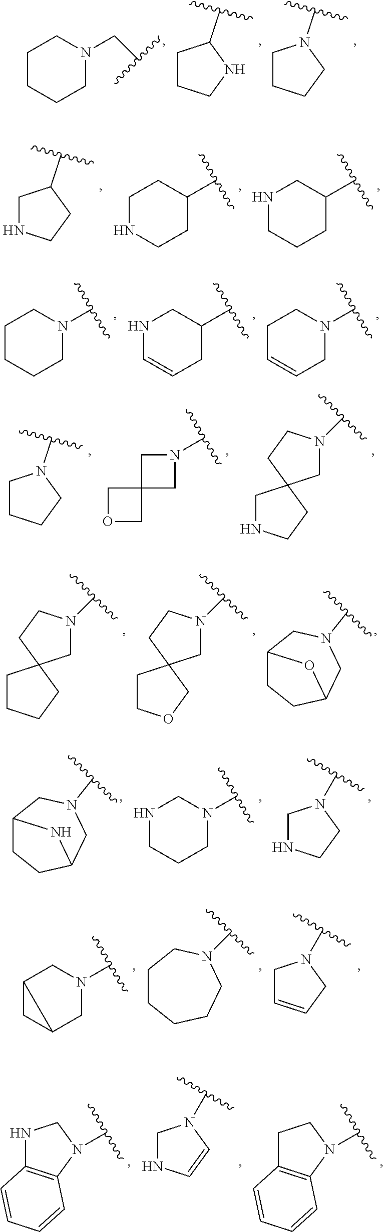

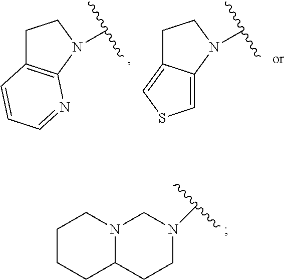 [SMILES: *N1CCc2cccnc21 ; *N1CCc2cscc21 ; *N1CCC2CCCCN2C1]

the expression “optionally substituted” refers to the case of being unsubstituted or substituted with one or more of the following substituents: F, Cl, Br, hydroxyl, cyano, oxo, methyl, ethyl, n-propyl, isopropyl, hydroxymethyl, hydroxyethyl, cyclopropyl, cyclobutyl, —CH₂OCH₃ and —CH₂CH₂OCH₃.

In preferred embodiments of the present invention, R^{e} is meta or para to a fused ring connected to a benzene ring; more preferably, R^{e} is para to a fused ring connected to a benzene ring;
still further preferably, the benzene ring connected to R^{D} and R^{e} is selected from the structure:

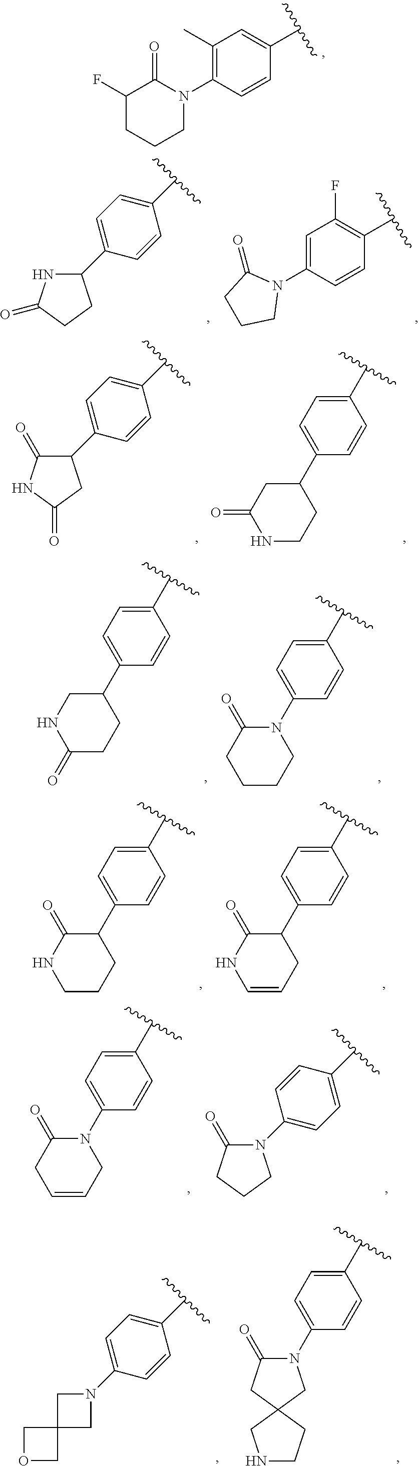

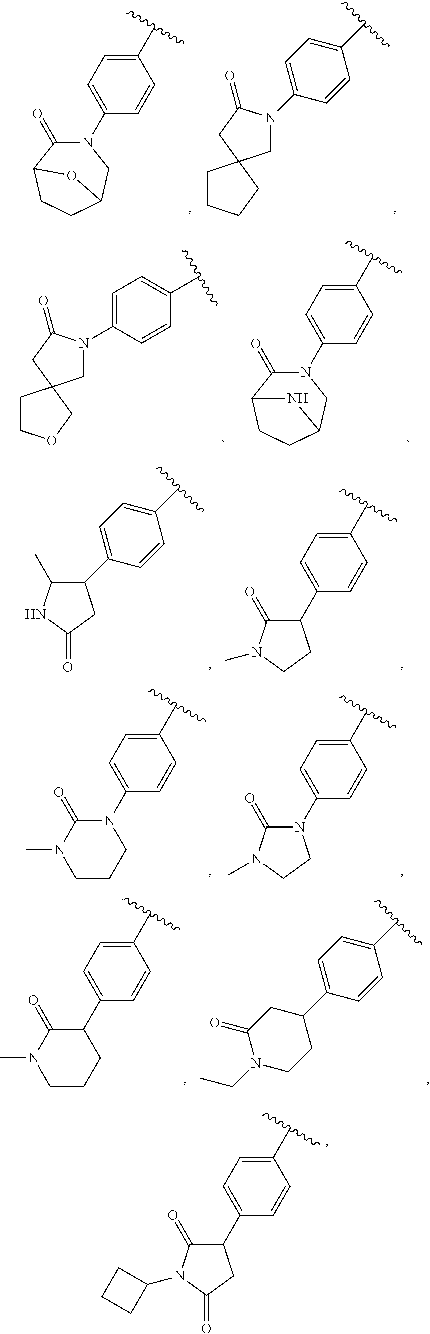

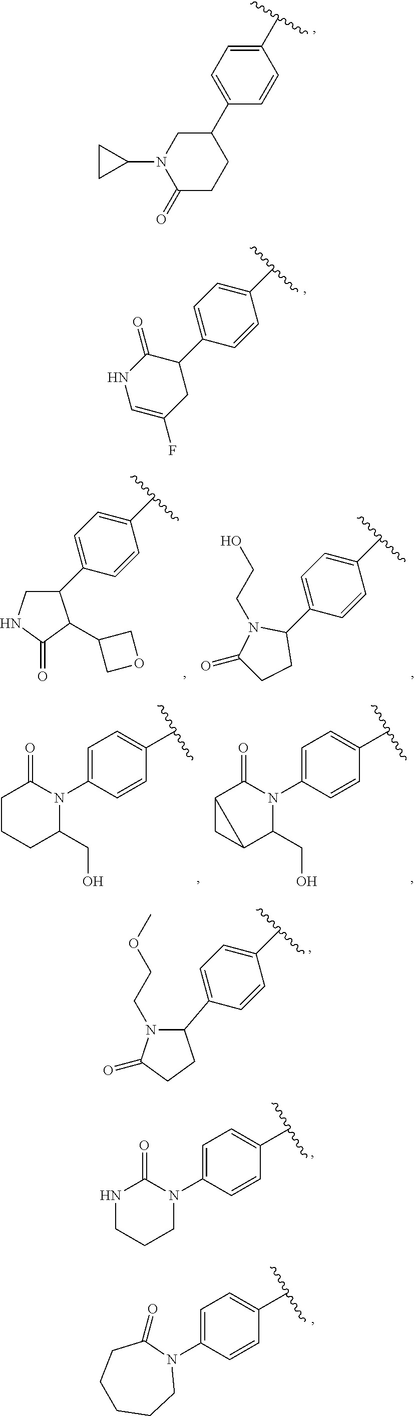

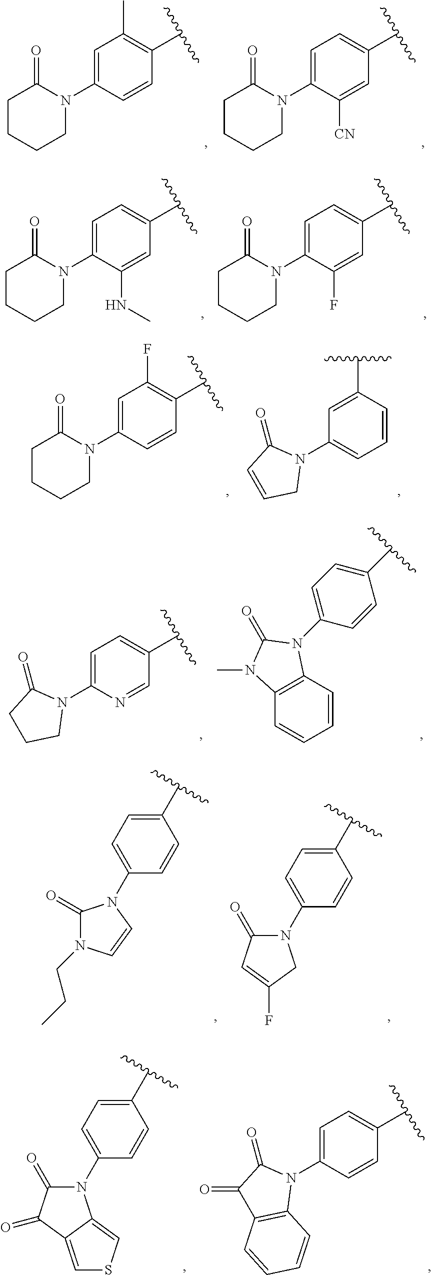

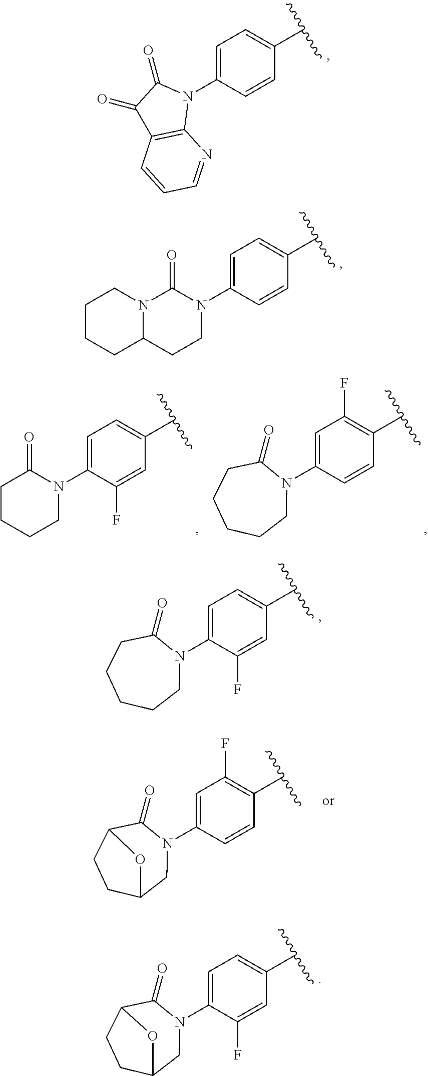

In a preferred embodiment of the present invention, the compound as shown in formula (X) is further represented by formula (XI):

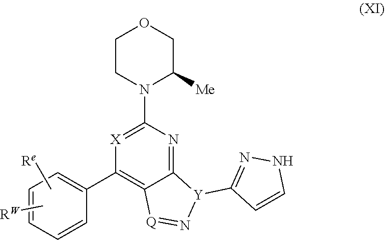

wherein the substituents in formula (XI) are as defined in formula (X).

The present invention further provides a compound as shown in formula (F), and a stereoisomer, an optical isomer, a pharmaceutical salt, a prodrug and a solvate thereof,

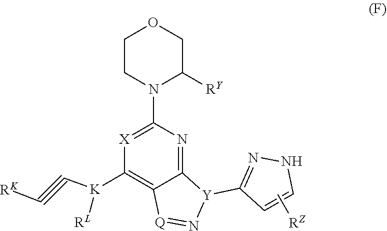

wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{Y} is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl;
K is selected from C₅₋₆ carbocyclyl or 5- to 7-membered heterocyclyl;
the number of R^{L} is 1 or 2, and R^{L}, at each occurrence, is independently selected from hydrogen, halogen, hydroxyl, cyano or C₁₋₃ alkyl;
R^{K} is selected from hydrogen, C₁₋₃ alkyl, —C(O) C₁₋₃ alkyl, halo C₁₋₃ alkyl, C₃₋₆ cycloalkyl, 5- to 6-membered heteroaryl, 5- to 8-membered heterocyclyl or phenyl, wherein the C₁₋₃ alkyl, —C(O) C₁₋₃ alkyl, halo C₁₋₃ alkyl, C₃₋₆ cycloalkyl, 5- to 6-membered heteroaryl, 5- to 8-membered heterocyclyl and phenyl are optionally substituted with substituents selected from hydroxyl, C₁₋₃ alkyl and halogen;
the heteroatoms in the “heterocyclyl” and “heteroaryl” are selected from N, O or S, and the number of the heteroatoms is 1, 2, 3 or 4.

In preferred embodiments of the present invention, Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio;
further preferably, Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio;
further preferably, Y is selected from N, and Q is selected from CR¹; R¹ is selected from hydrogen, halogen, cyano, amido, methyl, ethyl, methoxy, ethoxy, methylthio or ethylthio;
still further preferably, Y is selected from N, and Q is selected from CR¹; R¹ is selected from hydrogen, F, Cl, Br, cyano, amido, methyl, ethyl, methoxy or methylthio;
still further preferably, Y is selected from N, and Q is selected from CR¹; R¹ is selected from hydrogen, F, cyano, amido or methylthio;
most preferably, Y is selected from N, and Q is selected from CR¹; R¹ is selected from hydrogen.

In preferred embodiments of the present invention, X is selected from CR^{X}; wherein R^{X} is selected from hydrogen, halogen, hydroxyl, sulfhydryl, amino, cyano or C₁₋₆ alkyl;
further preferably, R^{X} is selected from hydrogen, F, Cl, Br, hydroxyl, amino, cyano or methyl;
still further preferably, R^{X} is selected from hydrogen.

In preferred embodiments of the present invention, R^{Y} is F, Cl, Br, methyl, ethyl, n-propyl, isopropyl or hydrogen;
further preferably, R^{Y} is F, Cl, Br, methyl, ethyl or hydrogen;
still further preferably, R^{Y} is methyl.

In preferred embodiments of the present invention, the number of R^{Z} is 0, 1, 2 or 3, and R^{Z}, at each occurrence, is independently selected from F, Cl, Br, hydroxyl, cyano, amino, methyl, ethyl, monofluoromethyl, difluoromethyl or trifluoromethyl;
further preferably, the number of R^{Z} is 0, 1 or 2, and R^{Z}, at each occurrence, is independently selected from F, Cl, Br, hydroxyl, methyl, monofluoromethyl, difluoromethyl or trifluoromethyl;
still further preferably, the number of R^{Z} is 0.

In preferred embodiments of the present invention, K is selected from cyclopentyl, cyclohexyl, 5-membered monocyclic heterocycloalkyl, 6-membered monocyclic heterocycloalkyl, 7-membered bridged heterocyclyl or cyclohexenyl;
further preferably, K is selected from cyclopentyl, cyclohexyl,

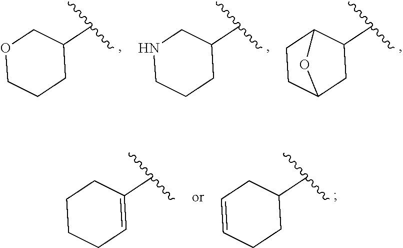 [SMILES: *C1CCCOC1 ; *C1CCCNC1 ; *C1CC2CCC1O2 ; *C1=CCCCC1 ; *C1CC=CCC1]

still further preferably, K is selected from cyclopentyl, cyclohexyl or

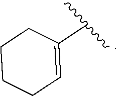 [SMILES: *C1=CCCCC1]

In preferred embodiments of the present invention, the number of R^{L} is 1 or 2, and R^{L} is selected from hydrogen, halogen, hydroxyl, cyano or methyl;
further preferably, the number of R^{L} is 1, and R^{L} is selected from hydrogen, hydroxyl or methyl;
still further preferably, the number of R^{L} is 1, and R^{L} is selected from hydrogen or hydroxyl.

In preferred embodiments of the present invention, R^{K} is selected from hydrogen, methyl, ethyl, n-propyl, isopropyl, —C(O) CH₃, —C(O) CH₂CH₃, C₃₋₆ monocyclic cycloalkyl, 5- to 6-membered monocyclic heteroaryl, 5- to 6-membered monocyclic heterocyclyl, 6- to 8-membered spiro heterocyclyl or phenyl, wherein the methyl, ethyl, n-propyl, isopropyl, —C(O) CH₃, —C(O) CH₂CH₃, C₃₋₆ monocyclic cycloalkyl, 5- to 6-membered monocyclic heteroaryl, 5- to 6-membered monocyclic heterocyclyl, 6- to 8-membered spiro heterocyclyl and phenyl are optionally substituted with substituents selected from hydroxyl, methyl, ethyl and halogen;
further preferably, R^{K} is selected from hydrogen, methyl, ethyl, —C(O) CH₃, cyclopropyl, cyclobutyl, cyclopentyl, 5- to 6-membered monocyclic heteroaryl, 5- to 6-membered monocyclic heterocyclyl, 7-membered spiro heterocyclyl or phenyl, wherein the methyl, ethyl, —C(O) CH₃, cyclopropyl, cyclobutyl, cyclopentyl, 5- to 6-membered monocyclic heteroaryl, 5- to 6-membered monocyclic heterocyclyl, 7-membered spiro heterocyclyl and phenyl are optionally substituted with substituents selected from hydroxyl, methyl, ethyl and halogen;
still further preferably, R^{K} is selected from hydrogen, methyl, ethyl, —C(O) CH₃, cyclopropyl, cyclobutyl, imidazolyl, pyrazolyl, tetrahydrofuryl,

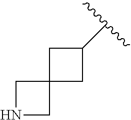 [SMILES: *C1CC2(CNC2)C1]

or phenyl, wherein the methyl, ethyl, —C(O) CH₃, cyclopropyl, imidazolyl, pyrazolyl, tetrahydrofuryl,

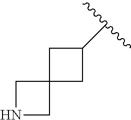 [SMILES: *C1CC2(CNC2)C1]

and phenyl are optionally substituted with substituents selected from hydroxyl, methyl, F, Cl and Br; even still further preferably, R^{K} is selected from hydrogen, methyl, hydroxymethyl, —CF₂CH₃, —C(O) CH₃, cyclopropyl,

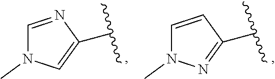

tetrahydrofuryl,

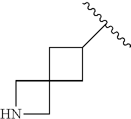 [SMILES: *C1CC2(CNC2)C1]

or phenyl; most preferably, R^{K} is selected from hydrogen.

In preferred embodiments of the present invention,

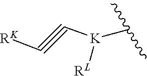

is selected from the following structure:

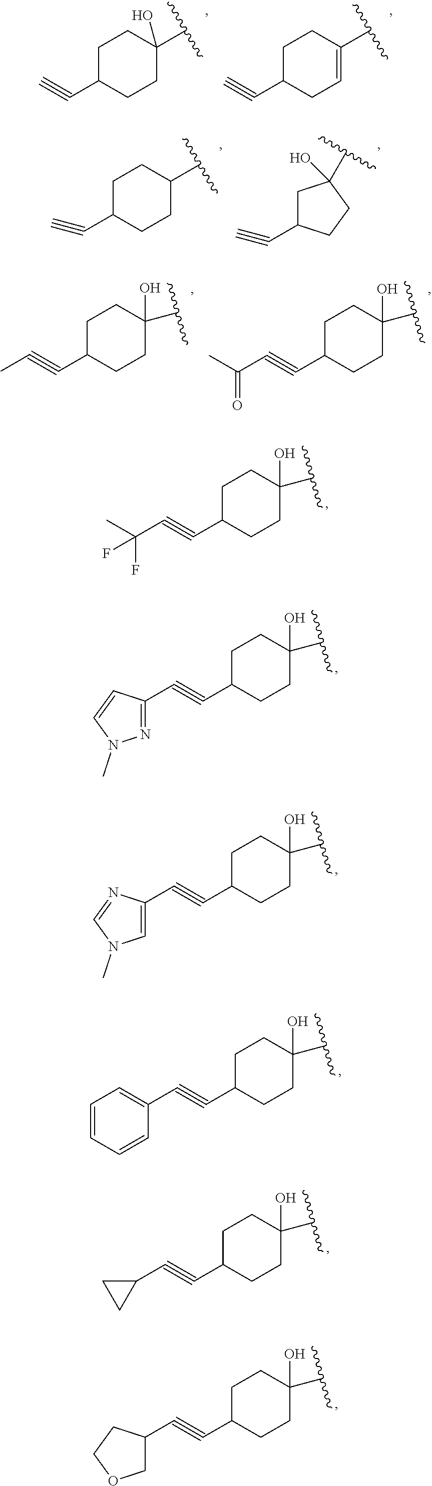

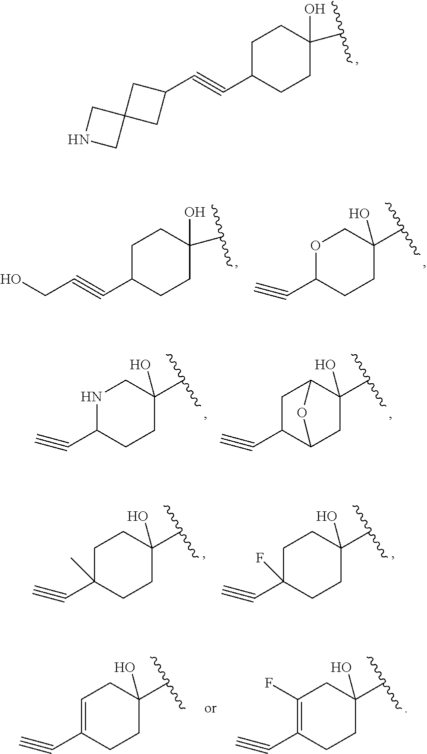

In a preferred embodiment of the present invention, the compound as shown in formula (F) is further represented by formula (F-1):

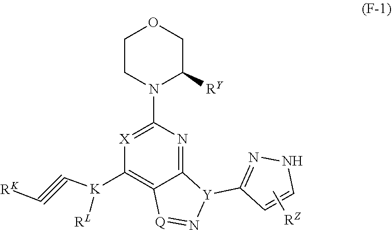

wherein the substituents in formula (F-1) are as defined in formula (F).

In a preferred embodiment of the present invention, the compound as shown in formula (F) is further represented by formula (F-2):

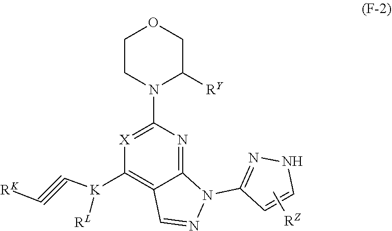

wherein the substituents in formula (F-2) are as defined in formula (F).

In a preferred embodiment of the present invention, the compound as shown in formula (F) is further represented by formula (F-3):

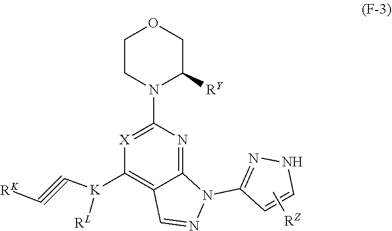

wherein the substituents in formula (F-3) are as defined in formula (F).

In a preferred embodiment of the present invention, the compound as shown in formula (F) is further represented by formula (F-4):

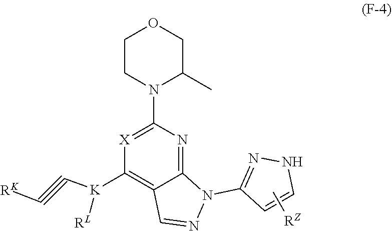

wherein the substituents in formula (F-4) are as defined in formula (F).

In a preferred embodiment of the present invention, the compound as shown in formula (F) is further represented by formula (F-5):

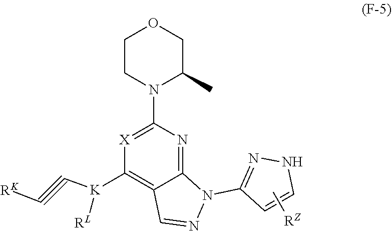

wherein the substituents in formula (F-5) are as defined in formula (F).

In a preferred embodiment of the present invention, the compound as shown in formula (F) further relates to a compound having a structure as shown in formula (F-3), and a stereoisomer, an optical isomer, a pharmaceutical salt, a prodrug and a solvate thereof:

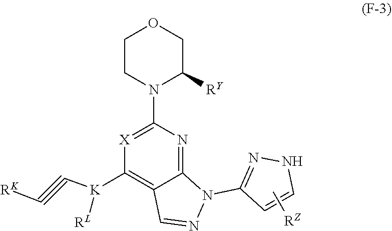

wherein R^{Y} is methyl;
X is CR^{X}, wherein R^{X} is hydrogen;
the number of R^{Z} s 0;

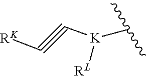

is selected from the following structure:

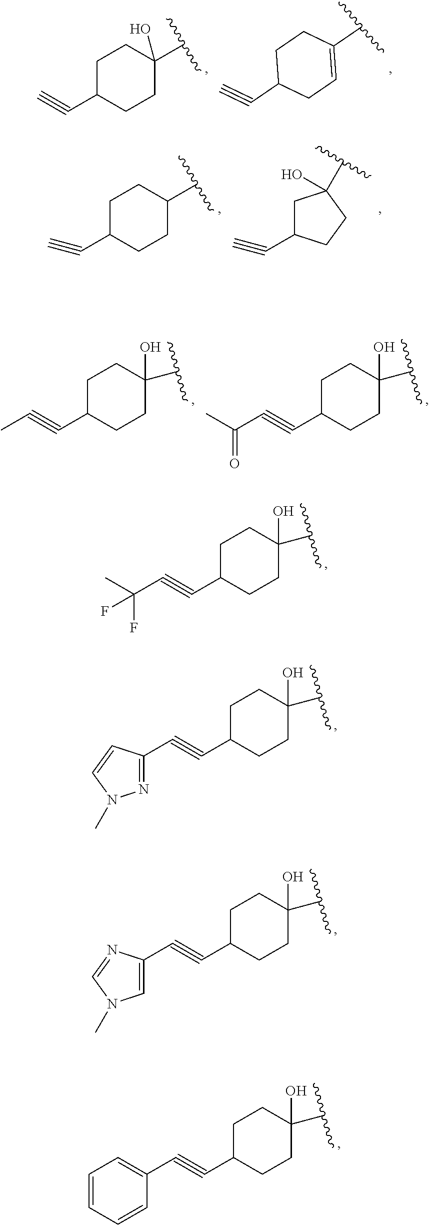

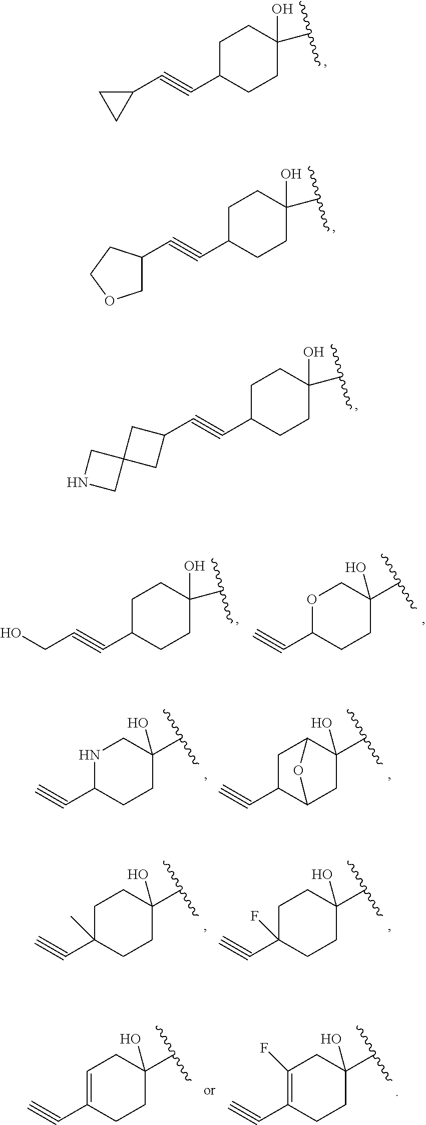

The compound of the present invention, and the stereoisomer, the optical isomer, the pharmaceutical salt, the prodrug and the solvate thereof, are selected from:

| No. | Structure |
|---|---|
| 1 | 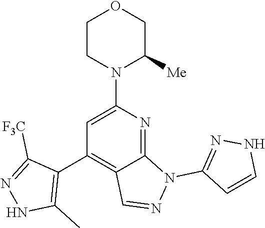 |
| 2 | 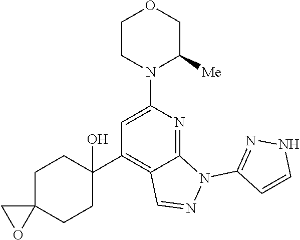 |
| 3 | 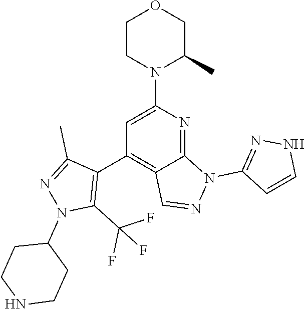 |
| 4 | 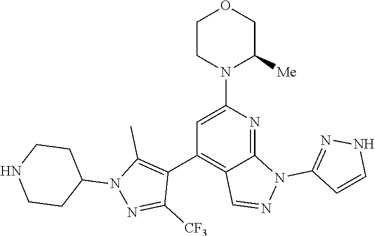 |
| 5 | 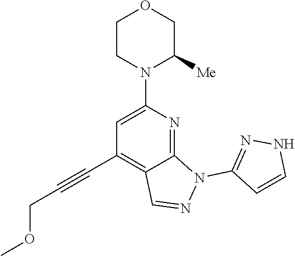 |
| 6 | 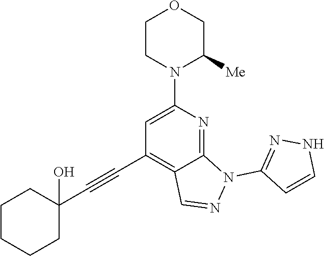 |
| 7 | 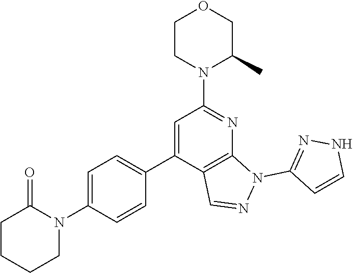 |
| 8 | 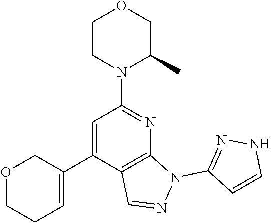 |
| 9 | 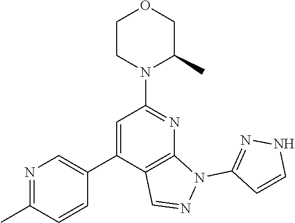 |
| 10 | 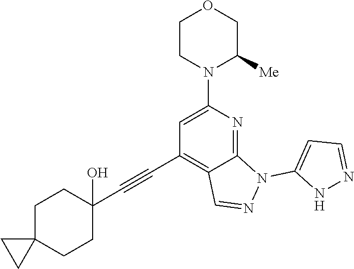 |
| 11 | 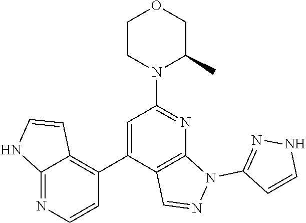 |
| 12 | 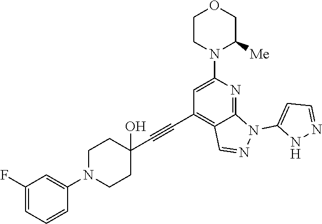 |
| 13 | 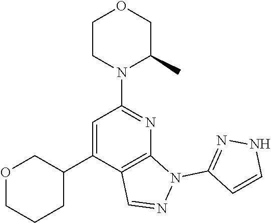 |
| 14 | 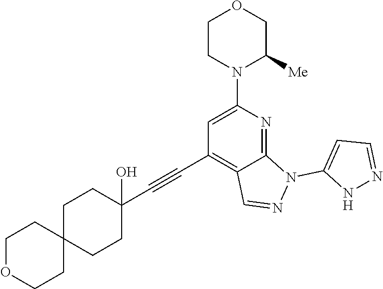 |
| 15 | 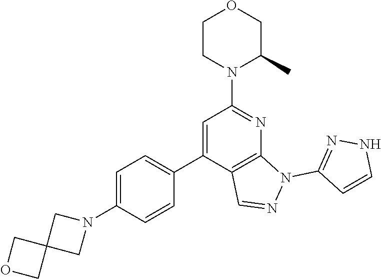 |
| 16 | 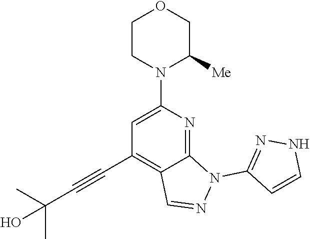 |
| 17 | 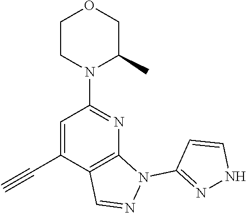 |
| 18 | 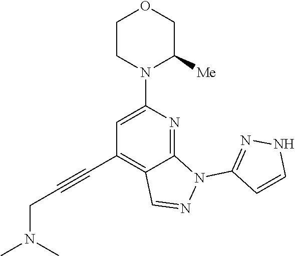 |
| 19 | 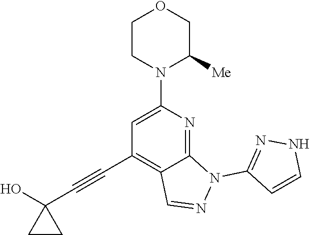 |
| 20 | 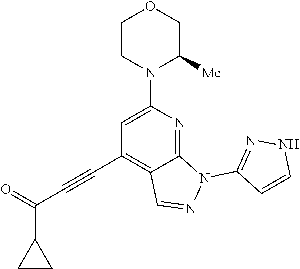 |
| 21 | 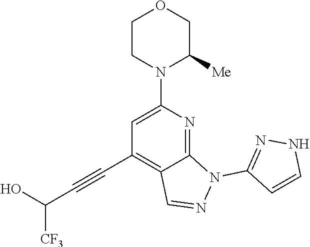 |
| 22 | 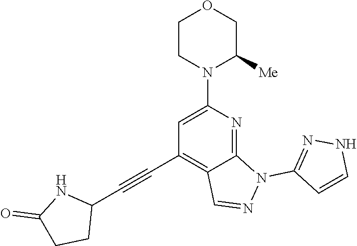 |
| 23 | 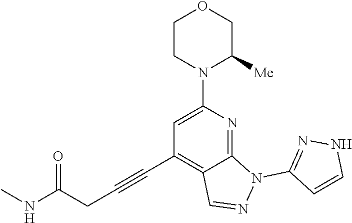 |
| 24 | 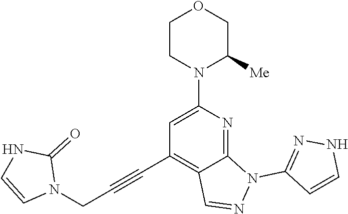 |
| 25 | 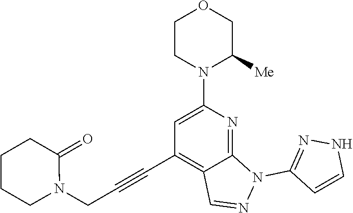 |
| 26 | 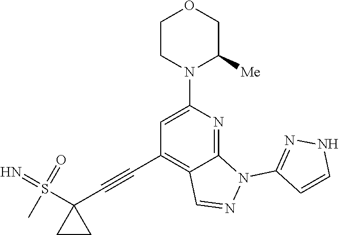 |
| 27 | 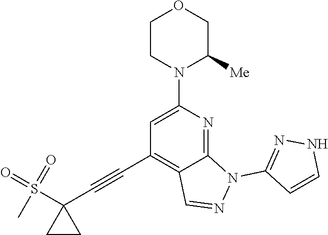 |
| 28 | 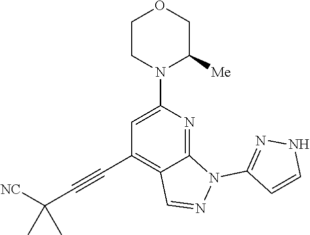 |
| 29 | 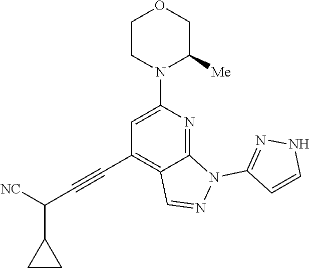 |
| 30 | 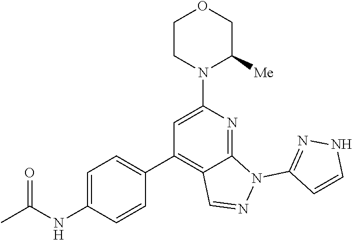 |
| 31 | 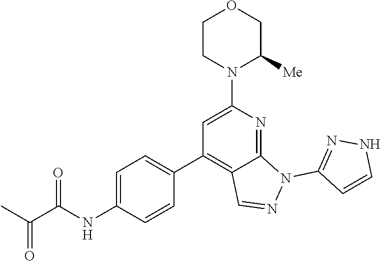 |
| 32 | 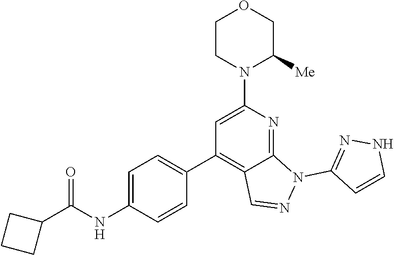 |
| 33 | 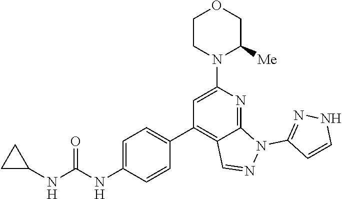 |
| 34 | 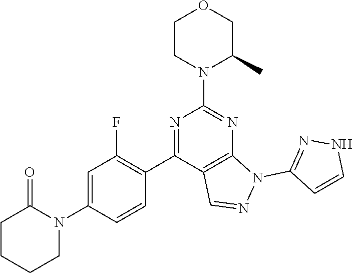 |
| 35 | 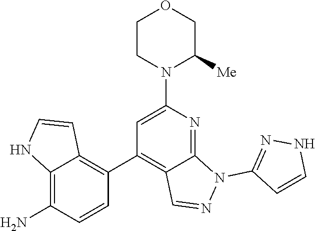 |
| 36 | 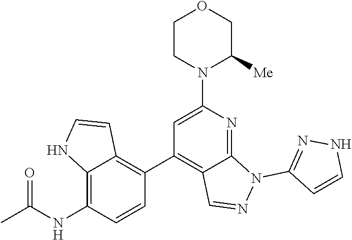 |
| 37 | 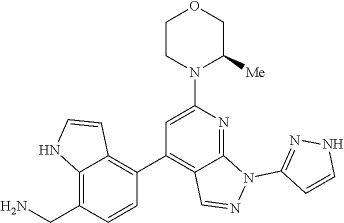 |
| 38 | 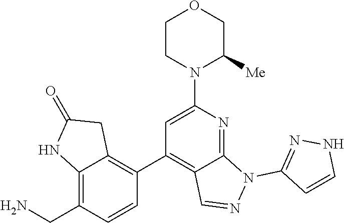 |
| 39 | 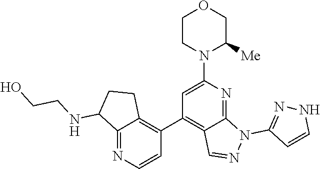 |
| 40 | 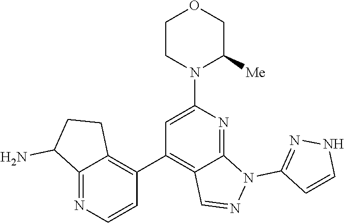 |
| 41 | 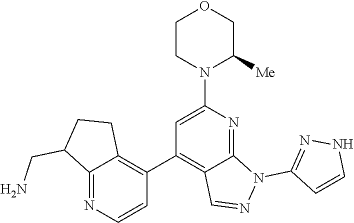 |
| 42 | 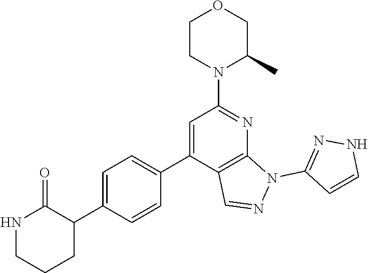 |
| 43 | 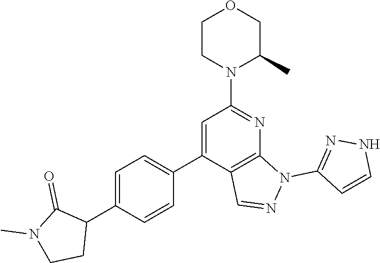 |
| 44 | 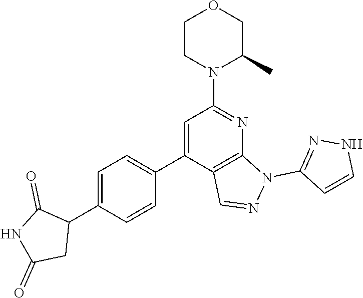 |
| 45 | 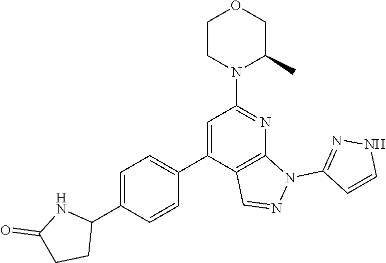 |
| 46 | 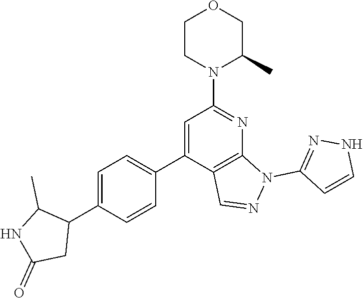 |
| 47 | 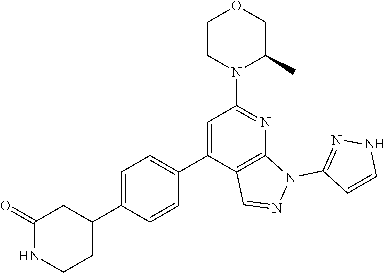 |
| 48 | 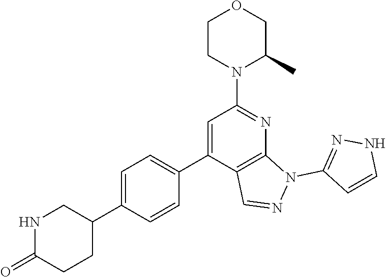 |
| 49 | 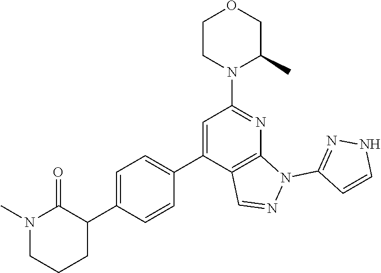 |
| 50 | 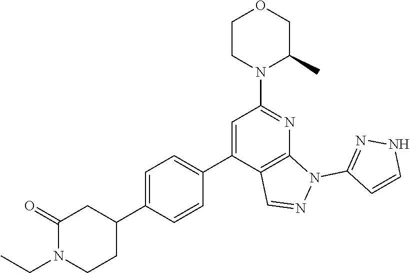 |
| 51 | 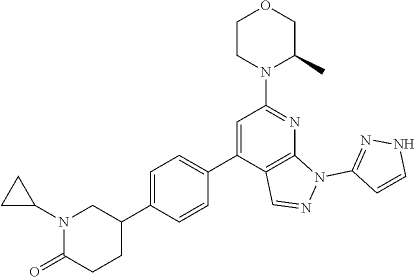 |
| 52 | 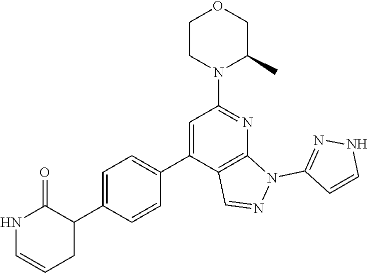 |
| 53 | 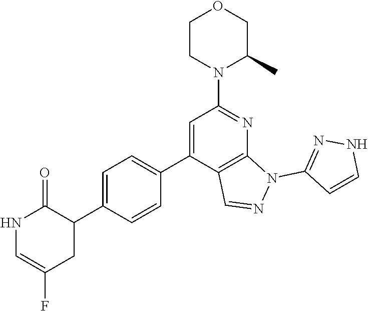 |
| 54 | 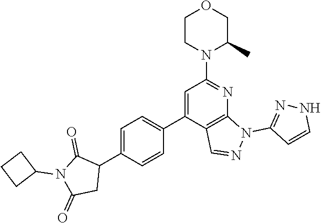 |
| 55 | 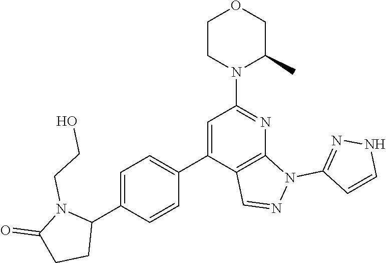 |
| 56 | 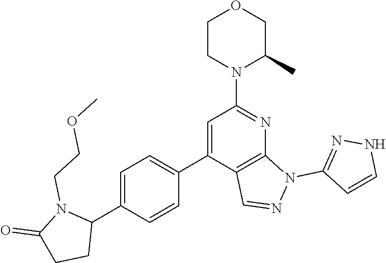 |
| 57 | 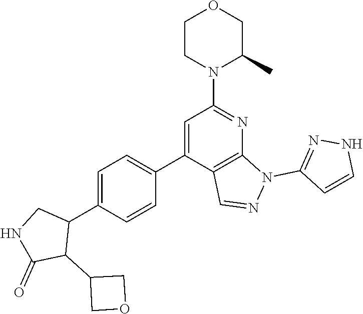 |
| 58 | 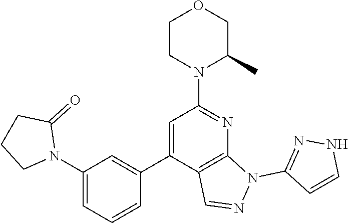 |
| 59 | 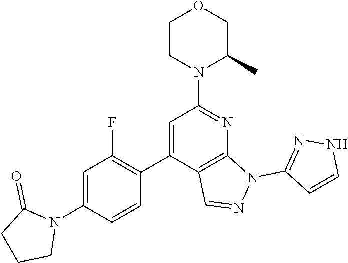 |
| 60 | 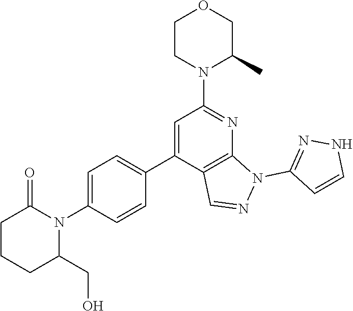 |
| 61 | 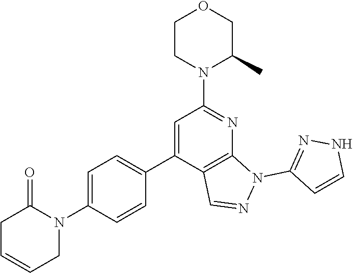 |
| 62 | 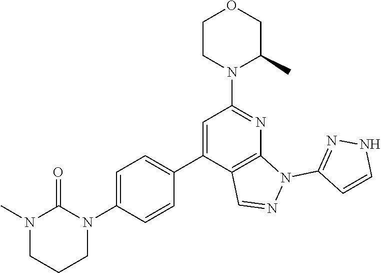 |
| 63 | 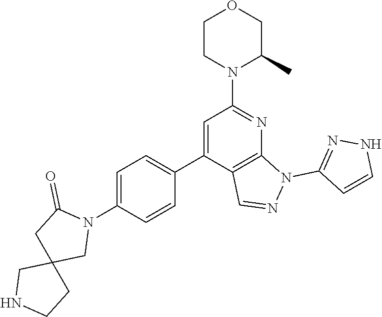 |
| 64 | 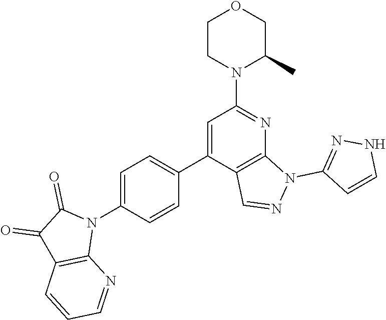 |
| 65 | 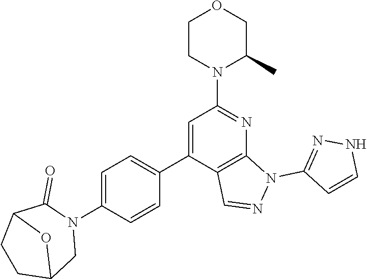 |
| 66 | 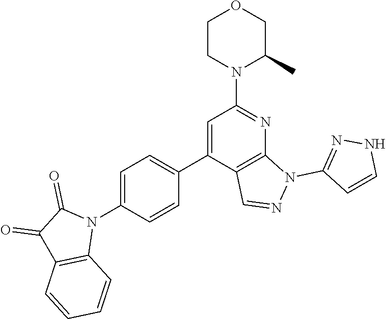 |
| 67 | 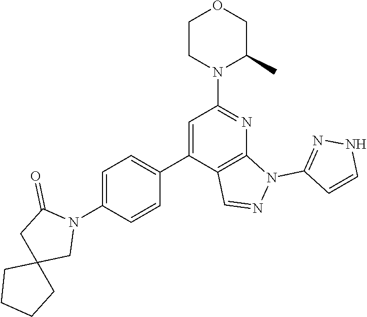 |
| 68 | 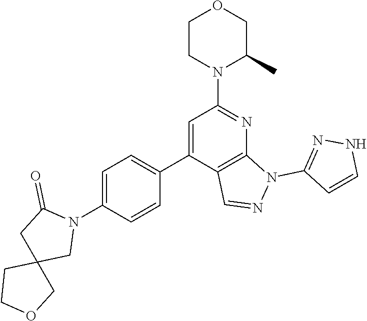 |
| 69 | 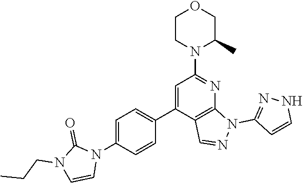 |
| 70 | 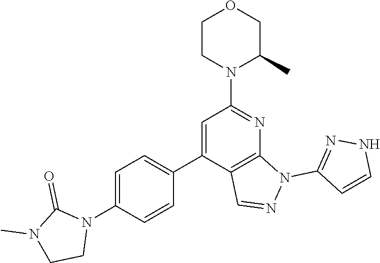 |
| 71 | 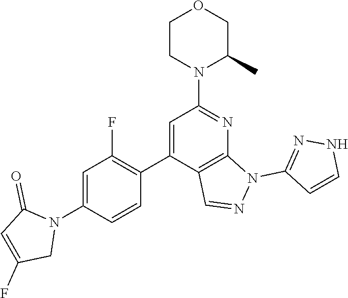 |
| 72 | 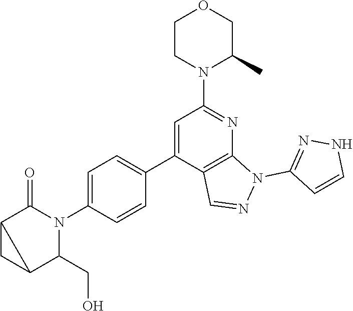 |
| 73 | 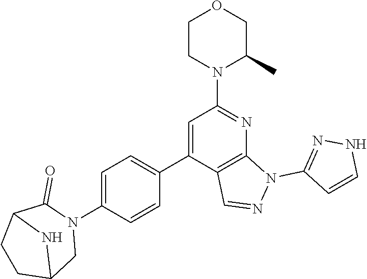 |
| 74 | 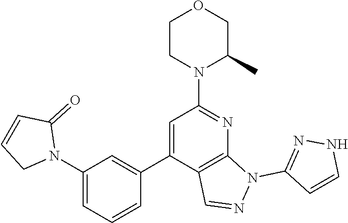 |
| 75 | 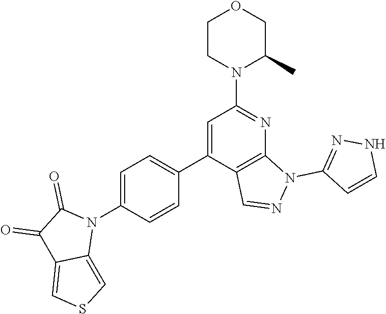 |
| 76 | 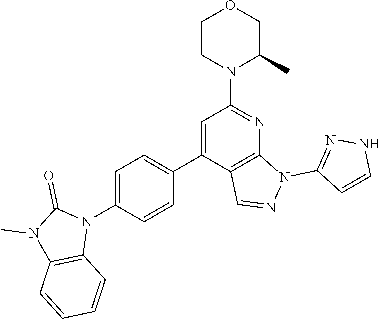 |
| 77 | 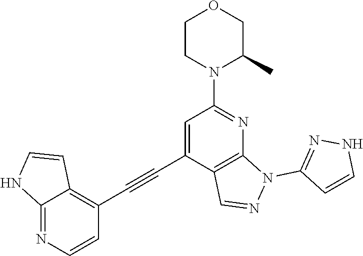 |
| 78 | 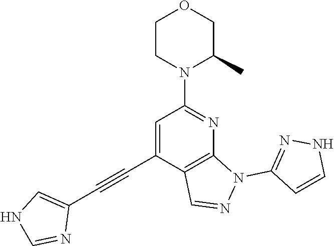 |
| 79 | 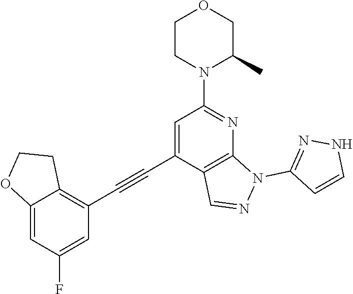 |
| 80 | 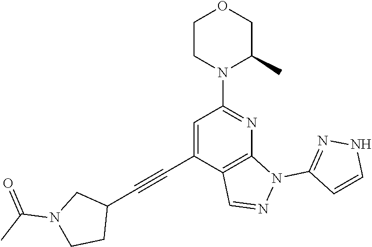 |
| 81 | 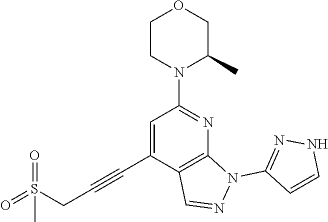 |
| 82 | 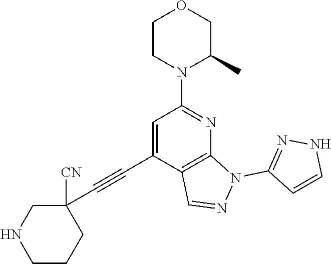 |
| 83 | 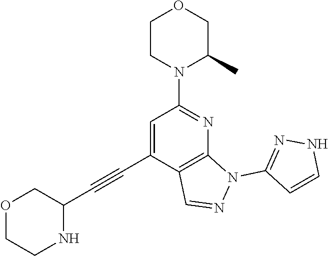 |
| 84 | 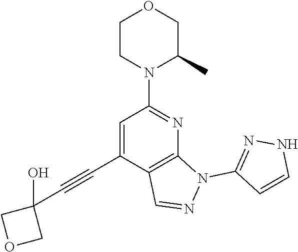 |
| 85 | 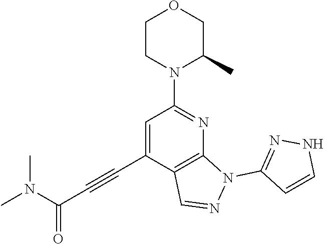 |
| 86 | 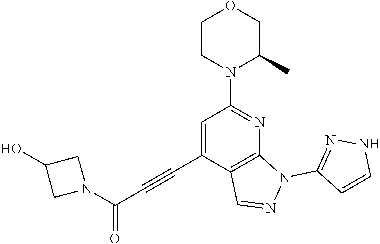 |
| 87 | 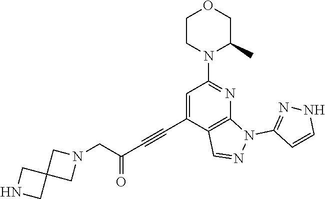 |
| 88 | 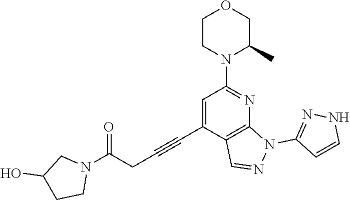 |
| 89 | 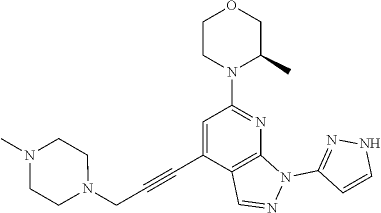 |
| 90 | 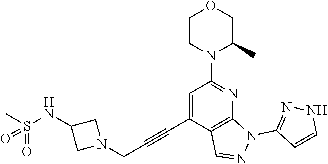 |
| 91 | 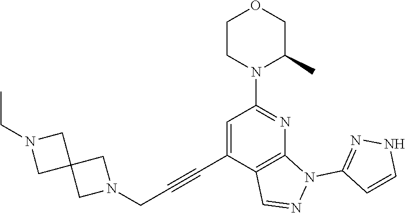 |
| 92 | 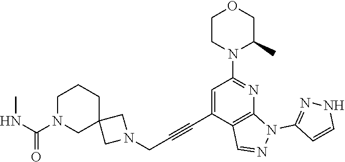 |
| 93 | 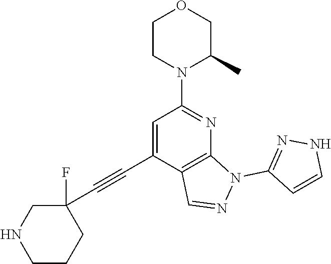 |
| 94 | 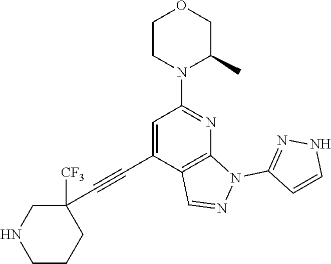 |
| 95 | 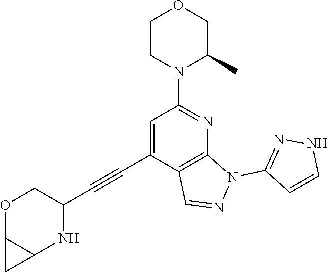 |
| 96 | 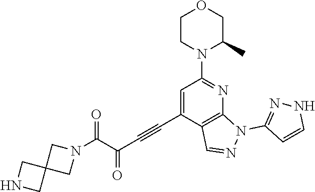 |
| 97 | 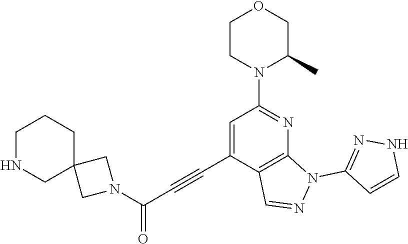 |
| 98 | 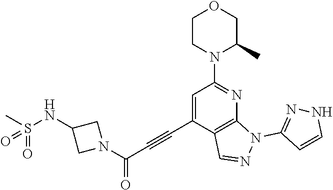 |
| 99 | 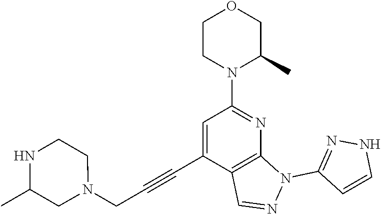 |
| 100 | 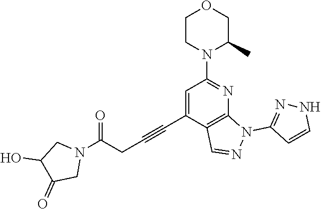 |
| 101 | 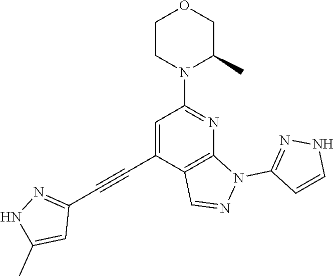 |
| 102 | 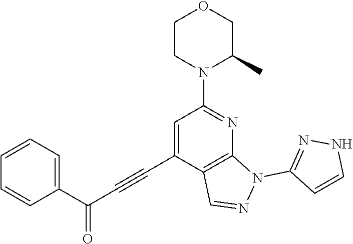 |
| 103 | 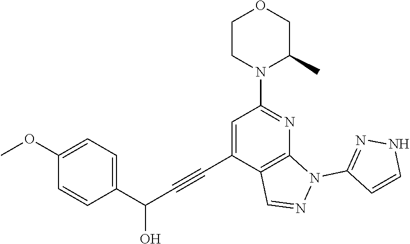 |
| 104 | 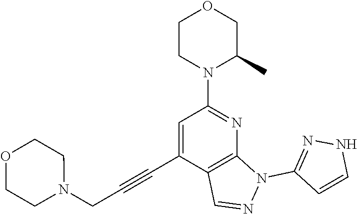 |
| 105 | 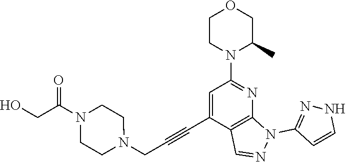 |
| 106 | 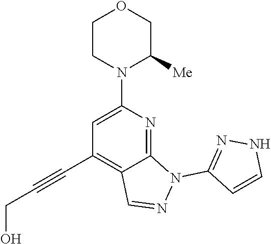 |
| 107 | 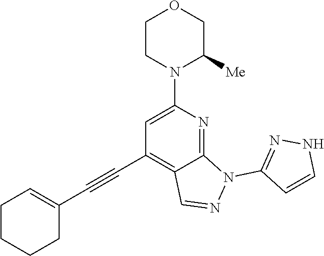 |
| 108 | 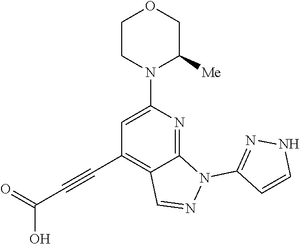 |
| 109 | 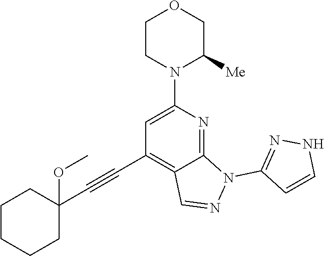 |
| 110 | 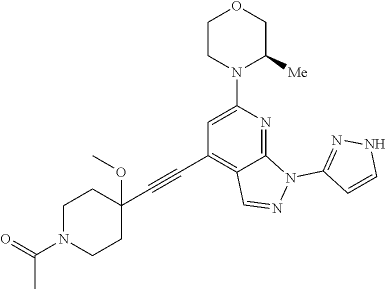 |
| 111 | 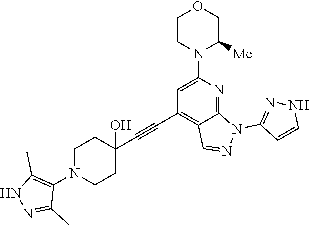 |
| 112 | 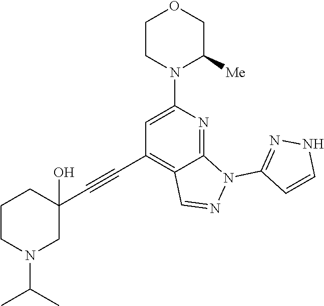 |
| 113 | 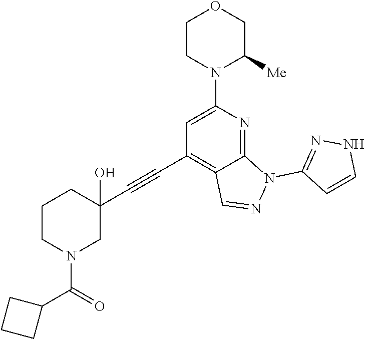 |
| 114 | 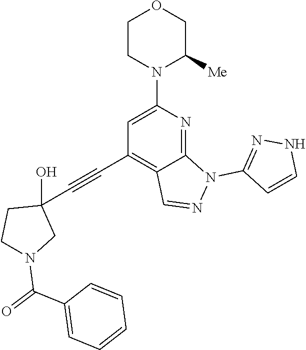 |
| 115 | 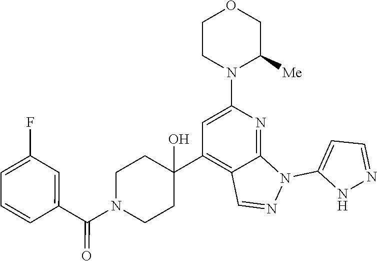 |
| 116 | 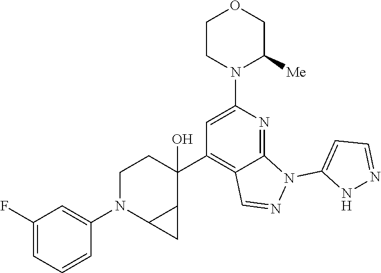 |
| 117 | 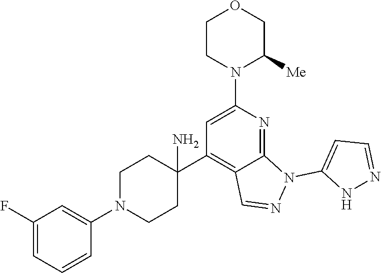 |
| 118 | 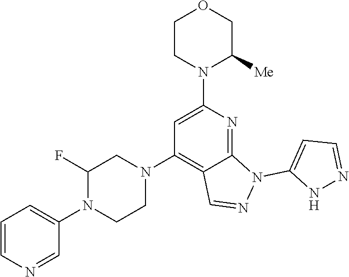 |
| 119 | 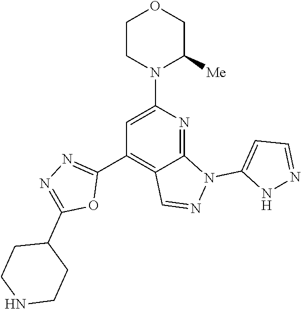 |
| 120 | 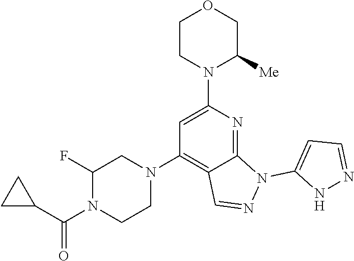 |
| 121 | 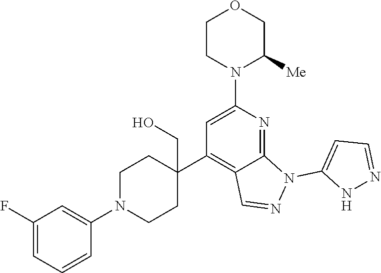 |
| 122 | 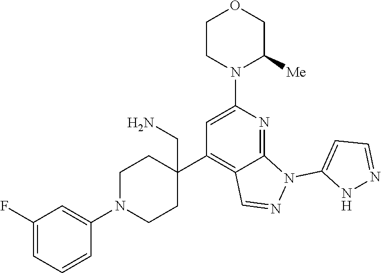 |
| 123 | 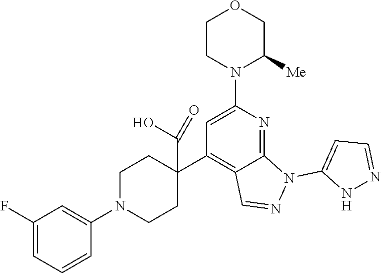 |
| 124 | 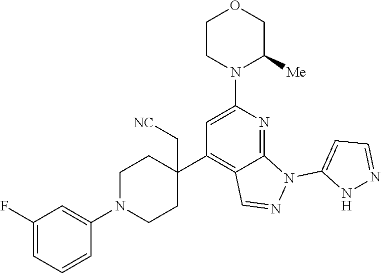 |
| 125 | 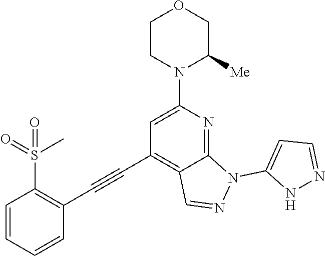 |
| 126 | 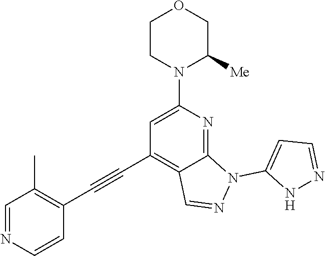 |
| 127 | 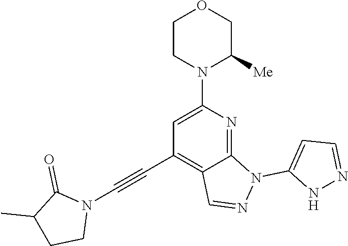 |
| 128 | 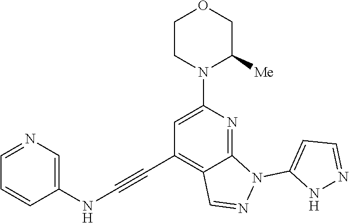 |
| 129 | 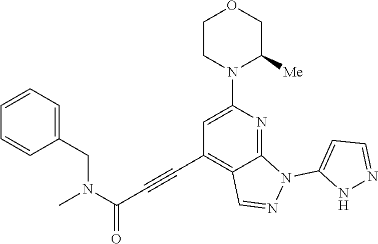 |
| 130 | 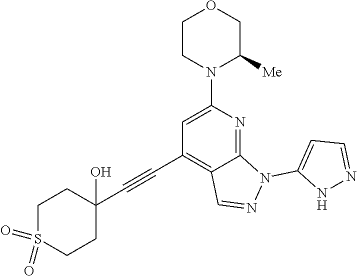 |
| 131 | 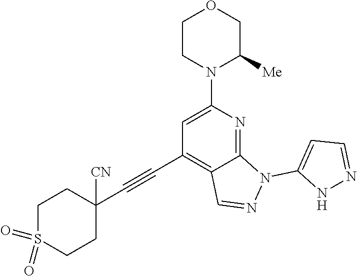 |
| 132 | 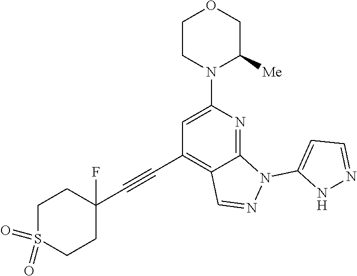 |
| 133 | 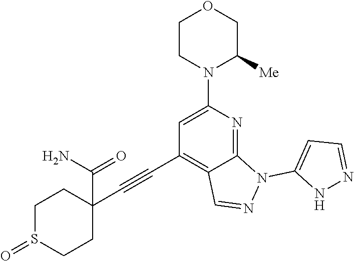 |
| 134 | 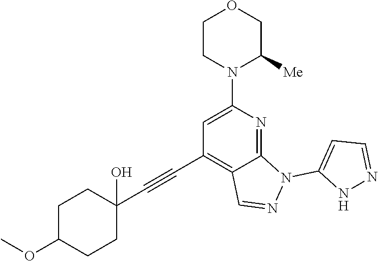 |
| 135 | 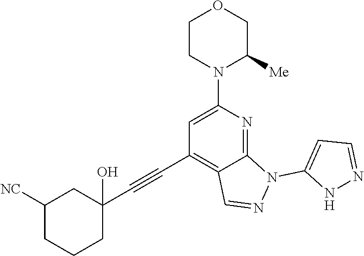 |
| 136 | 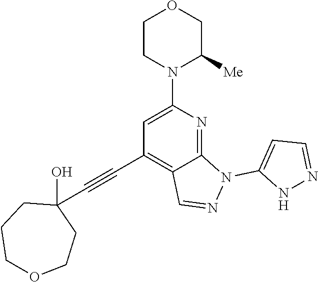 |
| 137 | 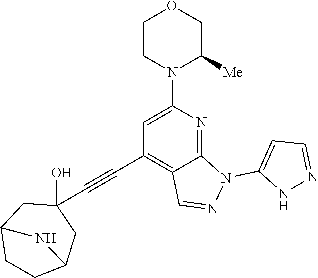 |
| 138 | 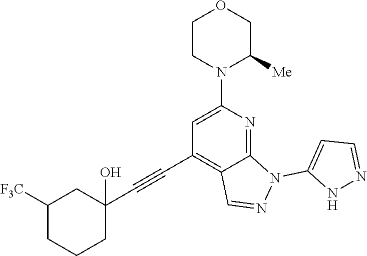 |
| 139 | 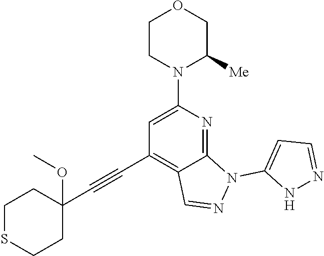 |
| 140 | 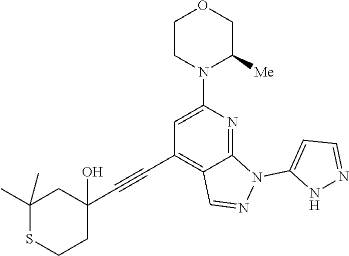 |
| 141 | 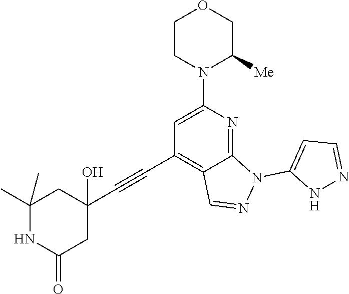 |
| 142 | 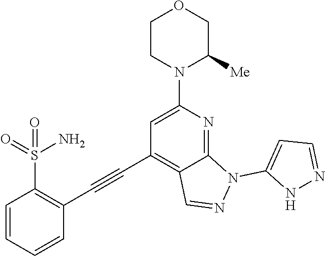 |
| 143 | 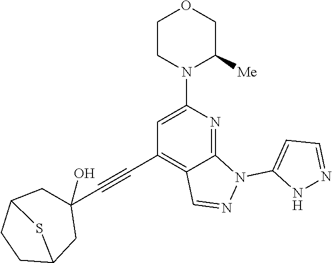 |
| 144 | 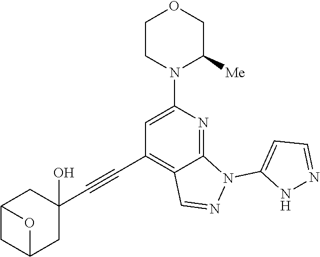 |
| 145 | 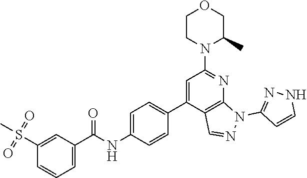 |
| 146 | 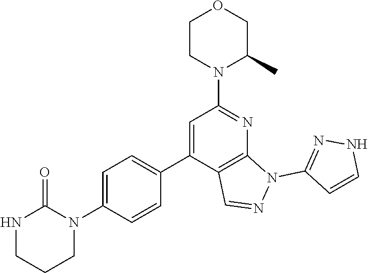 |
| 147 | 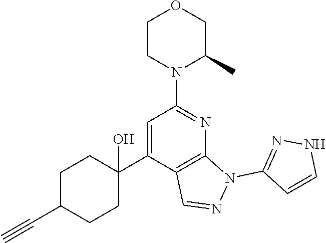 |
| 148 | 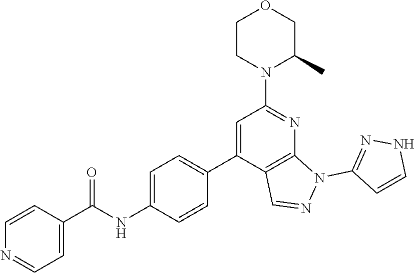 |
| 149 | 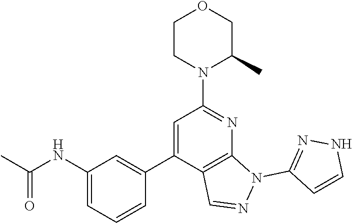 |
| 150 | 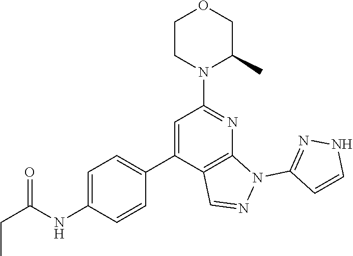 |
| 151 | 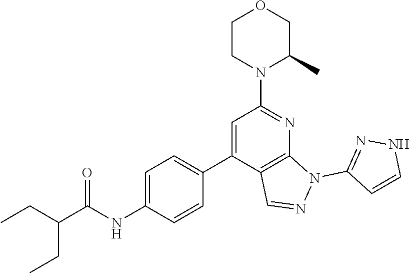 |
| 152 | 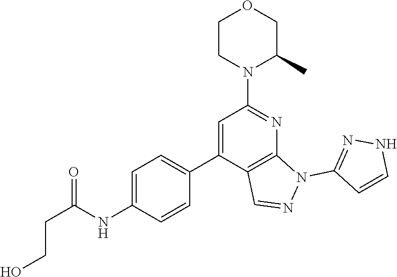 |
| 153 | 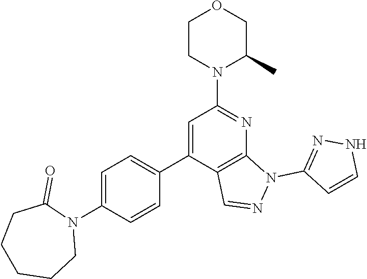 |
| 154 | 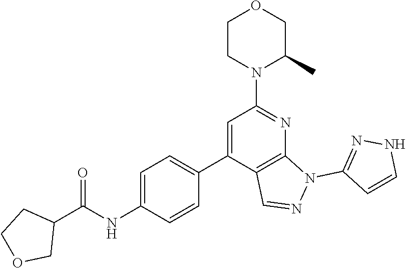 |
| 155 | 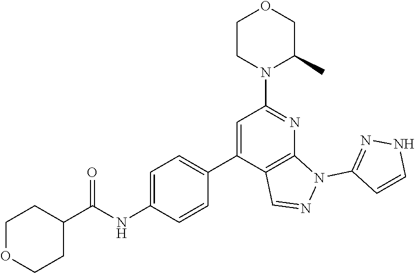 |
| 156 | 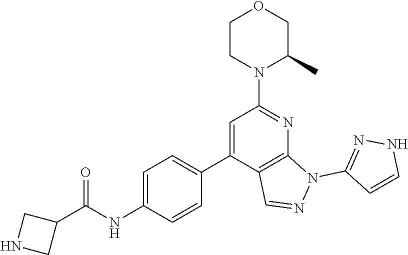 |
| 157 | 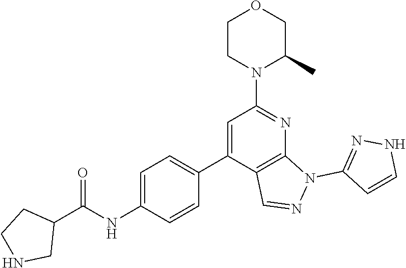 |
| 158 | 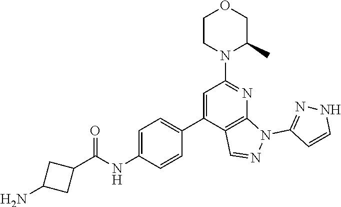 |
| 159 | 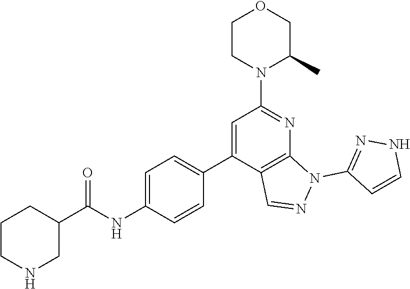 |
| 160 | 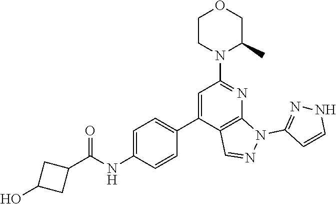 |
| 161 | 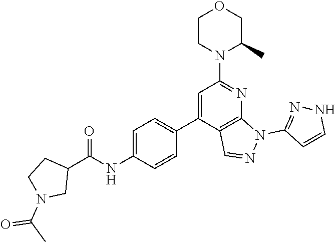 |
| 162 | 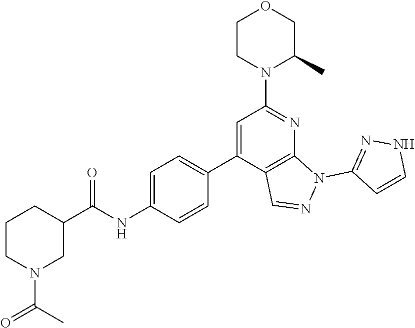 |
| 163 | 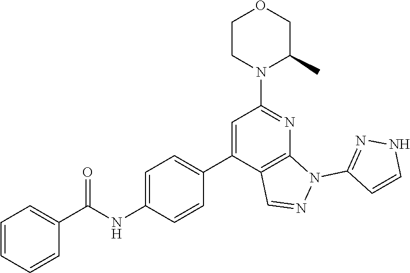 |
| 164 | 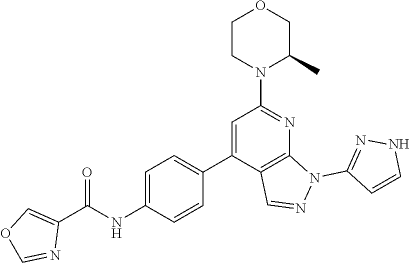 |
| 165 | 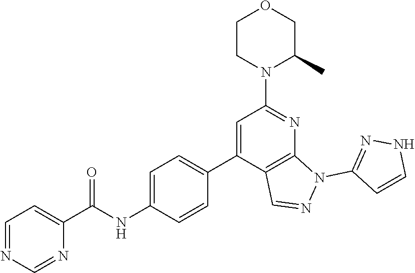 |
| 166 | 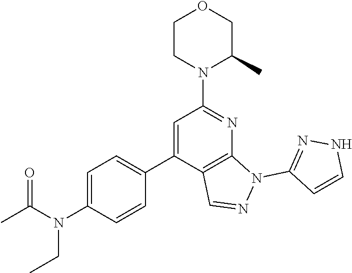 |
| 167 | 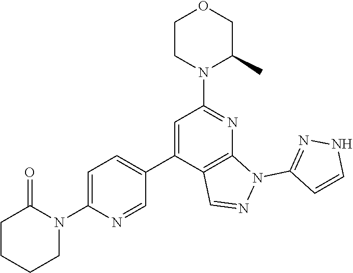 |
| 168 | 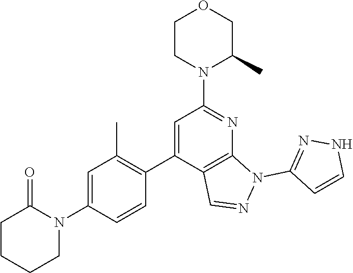 |
| 169 | 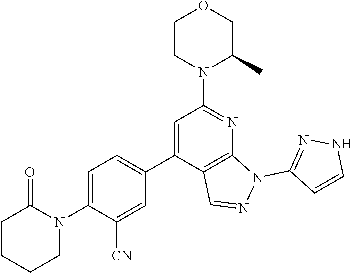 |
| 170 | 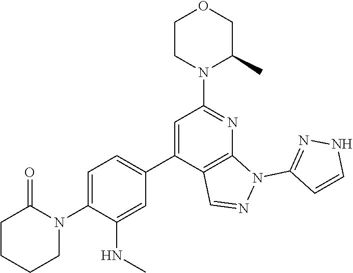 |
| 171 | 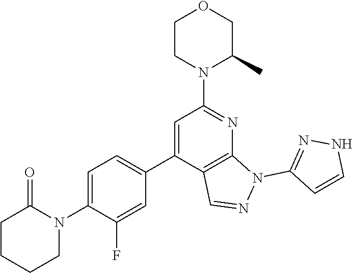 |
| 172 | 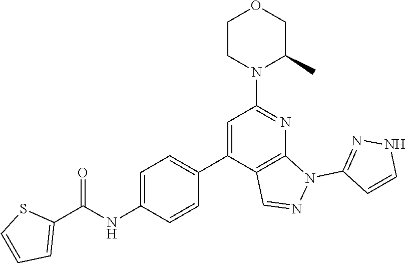 |
| 173 | 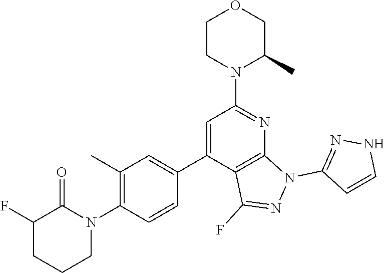 |
| 174 | 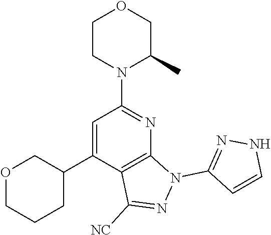 |
| 175 | 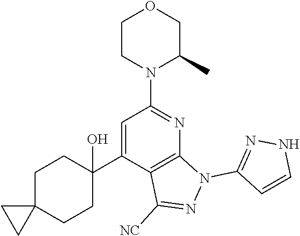 |
| 176 | 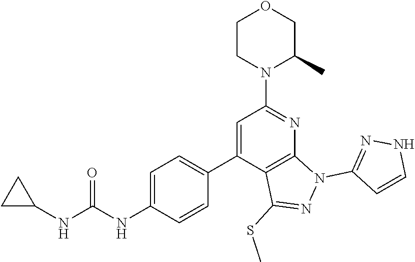 |
| 177 | 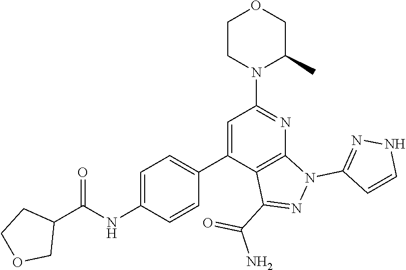 |
| 178 | 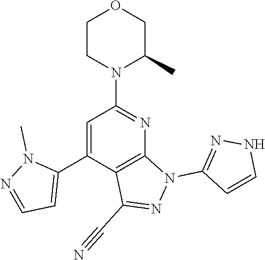 |
| 179 | 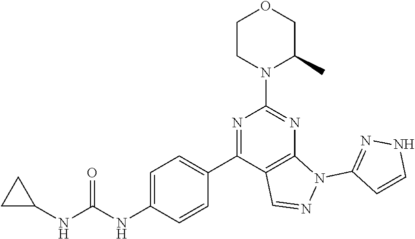 |
| 180 | 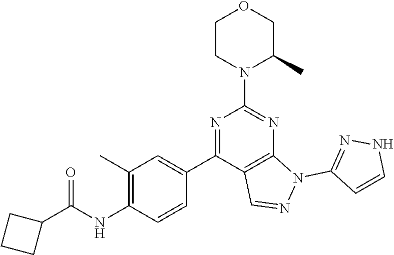 |
| 181 | 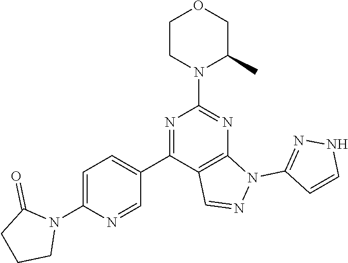 |
| 182 | 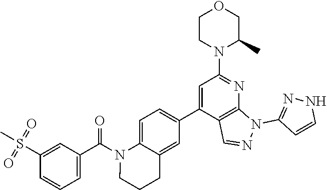 |
| 183 | 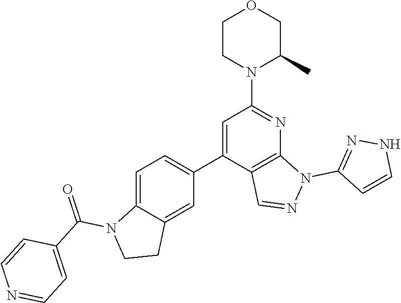 |
| 184 | 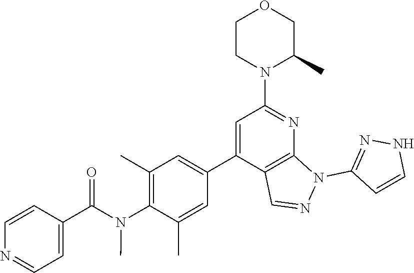 |
| 185 | 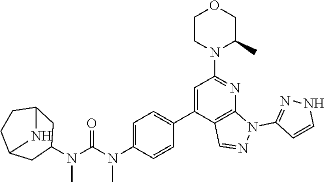 |
| 186 | 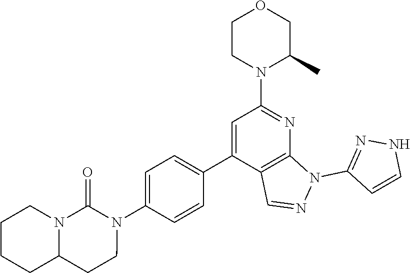 |
| 187 | 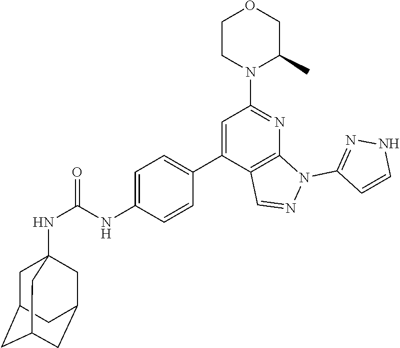 |
| 188 | 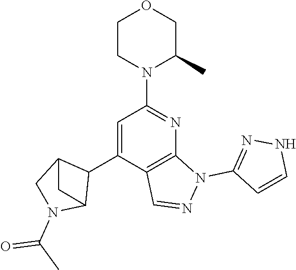 |
| 189 | 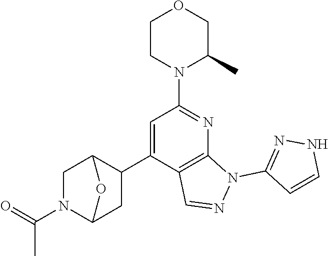 |
| 190 | 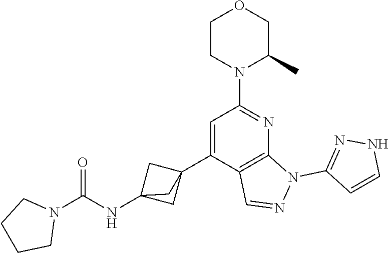 |
| 191 | 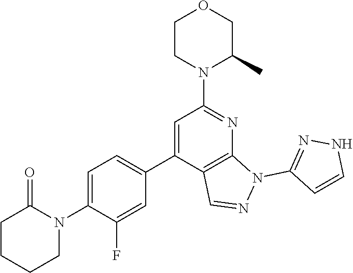 |
| 192 | 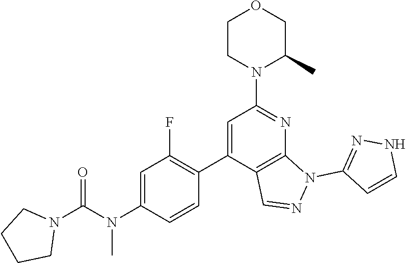 |
| 193 | 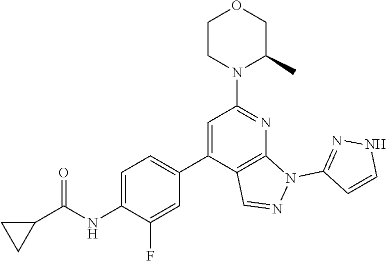 |
| 194 | 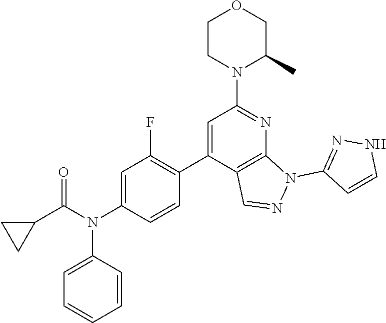 |
| 195 | 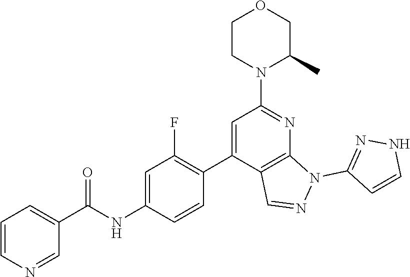 |
| 196 | 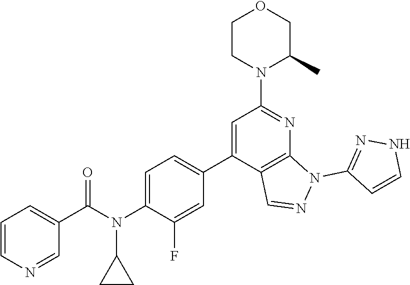 |
| 197 | 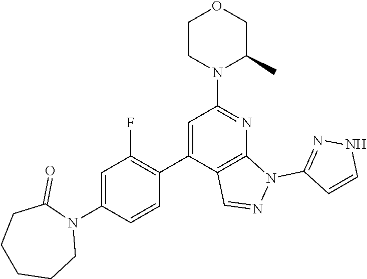 |
| 198 | 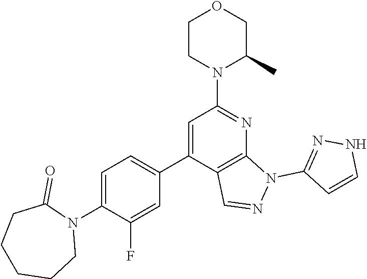 |
| 199 | 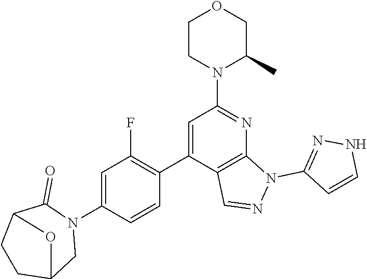 |
| 200 | 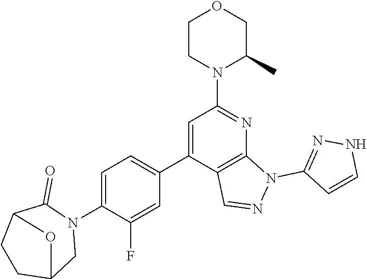 |
| 201 | 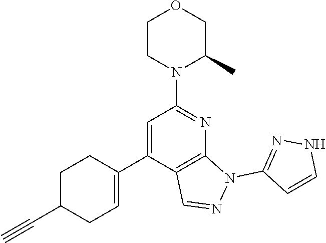 |
| 202 | 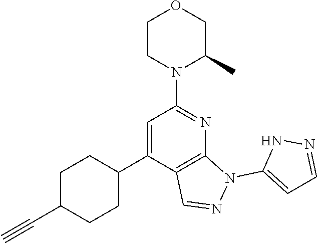 |
| 203 | 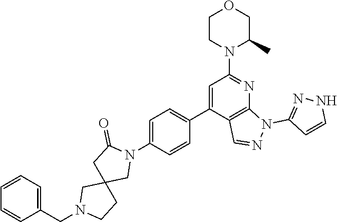 |
| 204 | 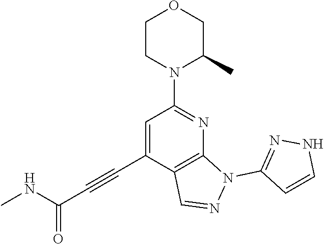 |
| 205 | 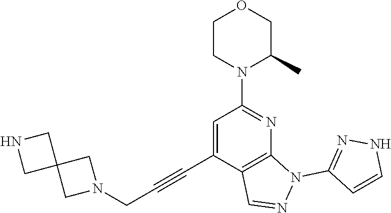 |
| 206 | 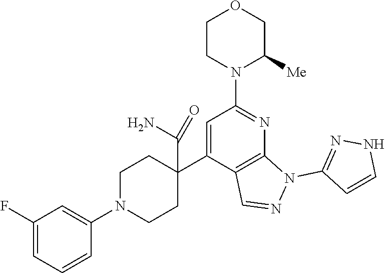 |
| 207 | 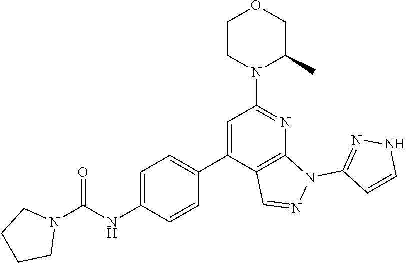 |
| 208 | 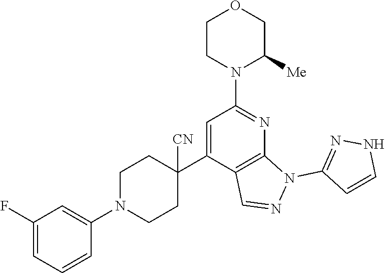 |
| 209 | 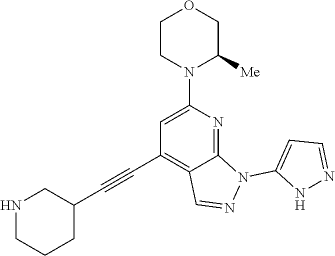 |
| 210 | 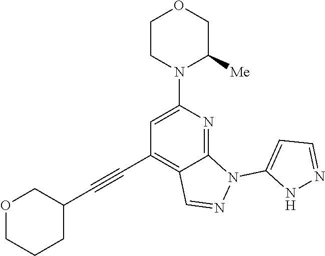 |
| 211 | 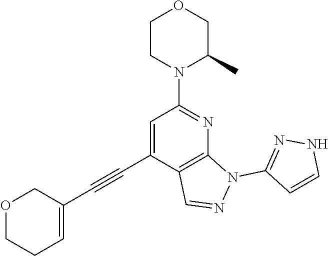 |
| 212 | 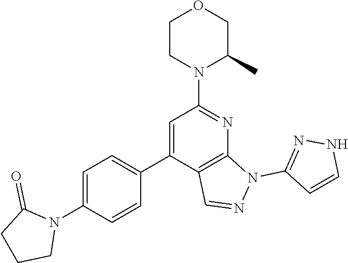 |
| 213 | 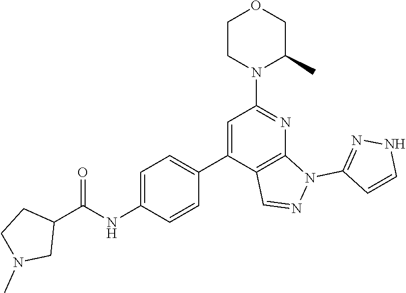 |
| 214 | 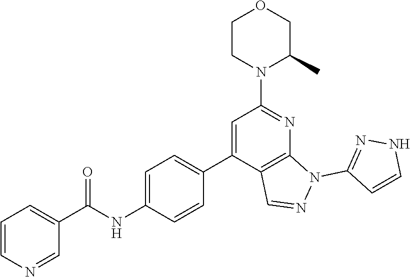 |
| 215 | 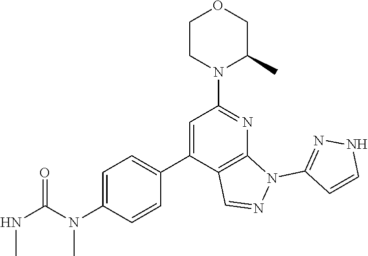 |
| 216 | 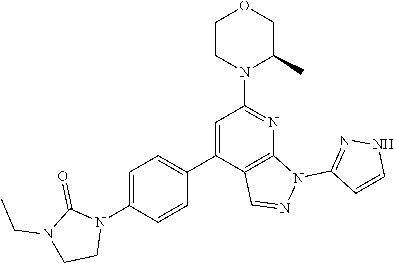 |
| 217 | 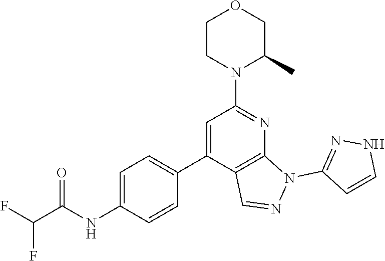 |
| 218 | 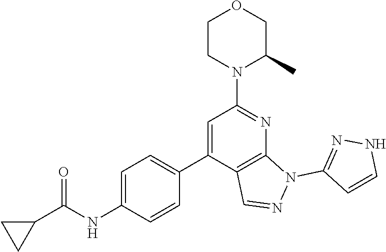 |
| 219 | 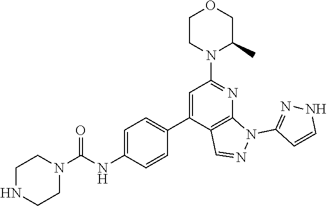 |
| 220 | 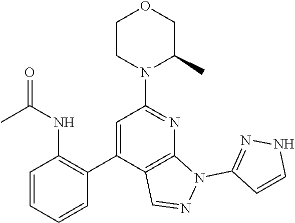 |
| 221 | 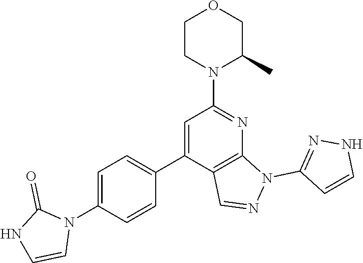 |
| 222 | 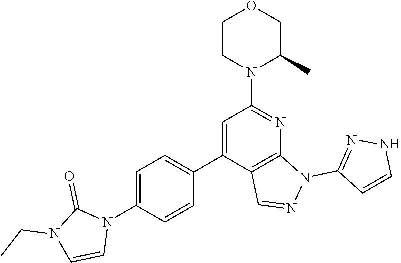 |
| 223 | 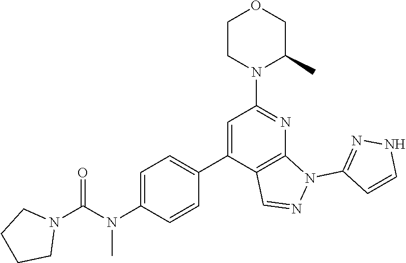 |
| 224 | 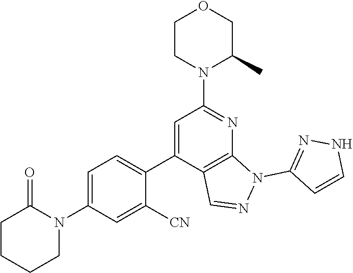 |
| 225 | 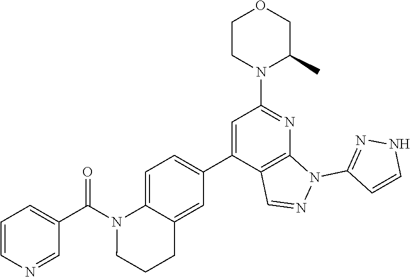 |
| 226 | 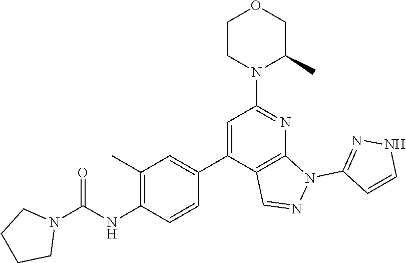 |
| 227 | 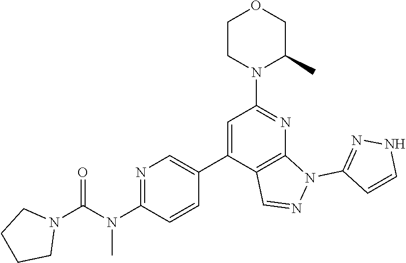 |
| 228 | 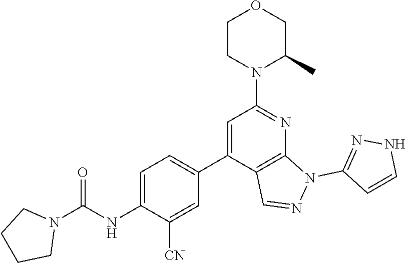 |
| 229 | 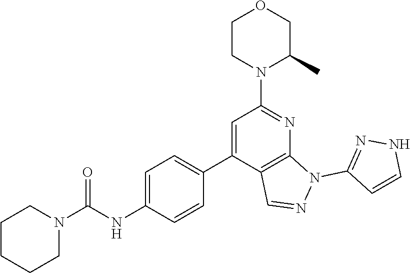 |
| 230 | 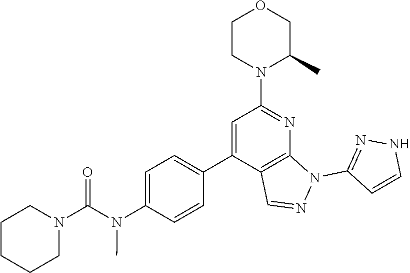 |
| 231 | 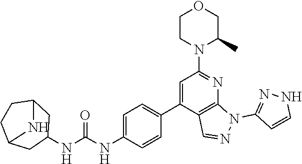 |
| 232 | 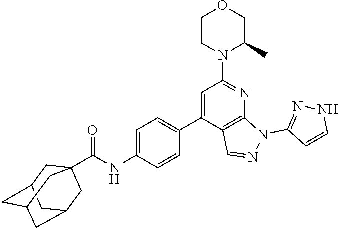 |
| 233 | 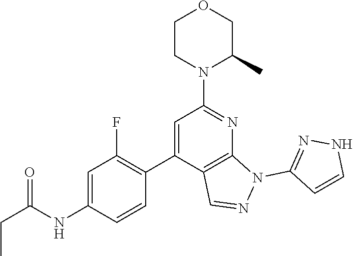 |
| 234 | 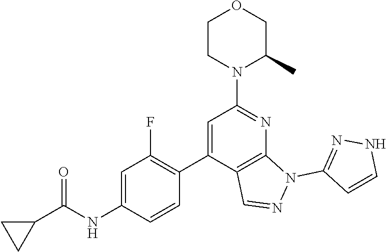 |
| 235 | 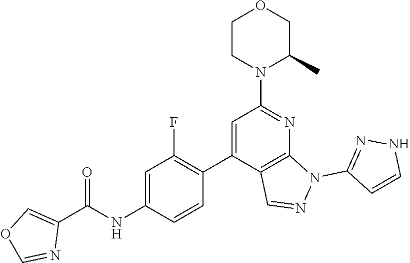 |
| 236 | 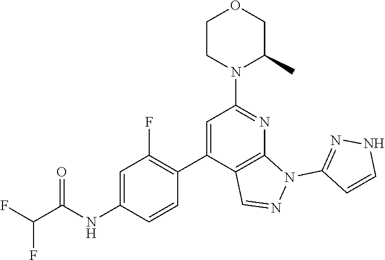 |
| 237 | 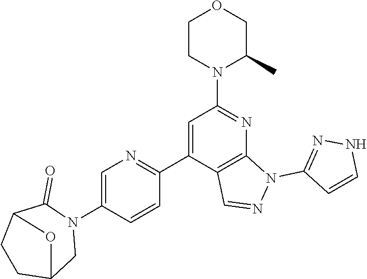 |
| 238 | 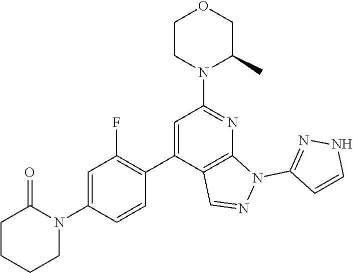 |
| 239 | 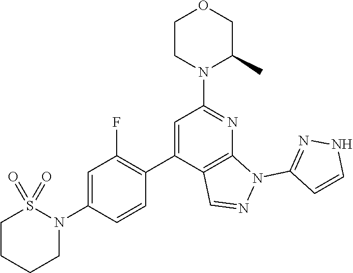 |
| 240 | 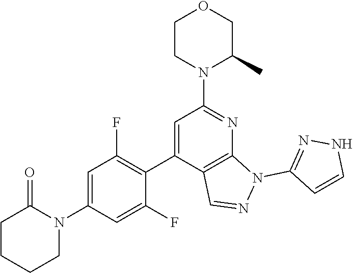 |
| 241 | 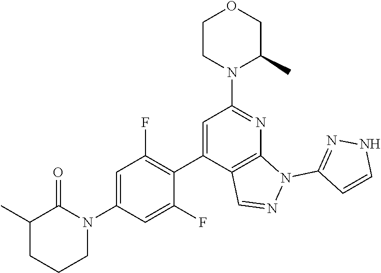 |
| 242 | 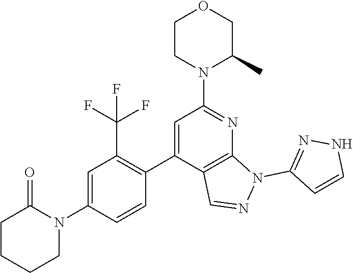 |
| 243 | 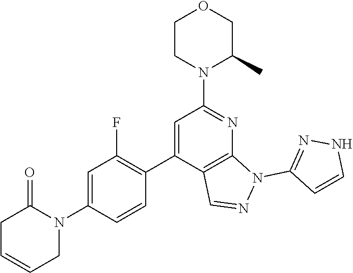 |
| 244 | 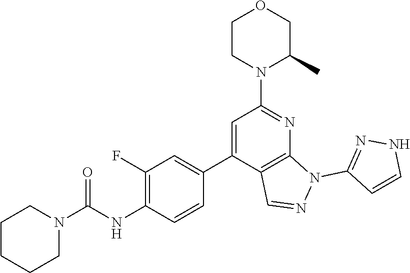 |
| 245 | 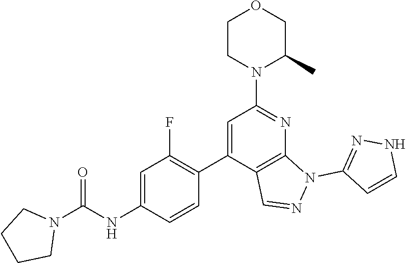 |
| 246 | 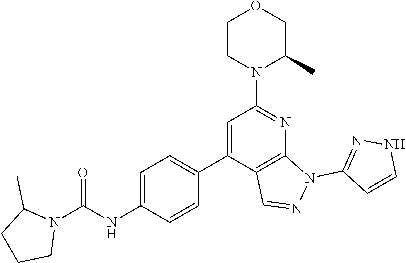 |
| 247 | 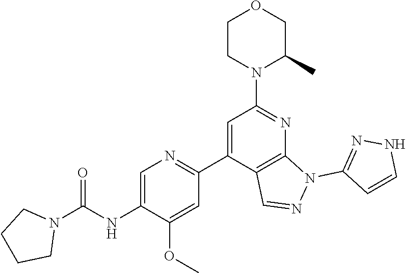 |
| 248 | 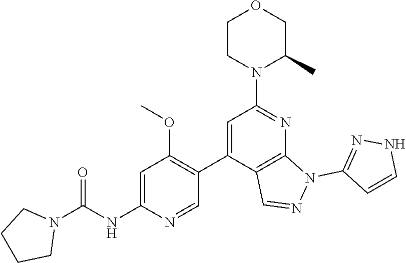 |
| 249 | 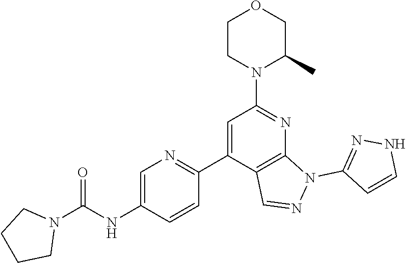 |
| 250 | 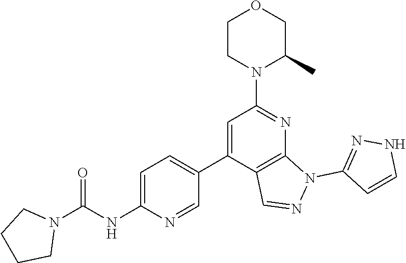 |
| 251 | 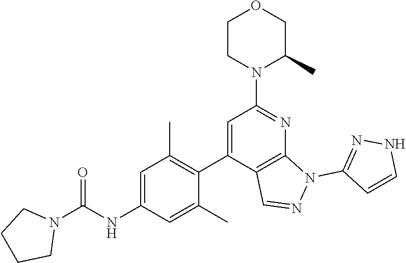 |
| 252 | 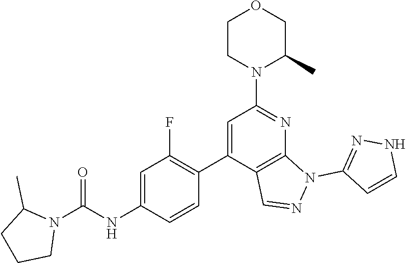 |
| 253 | 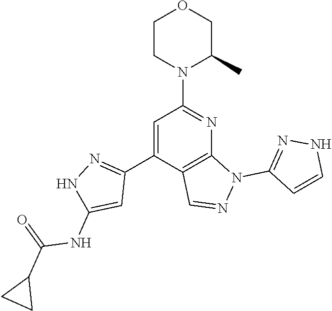 |
| 254 | 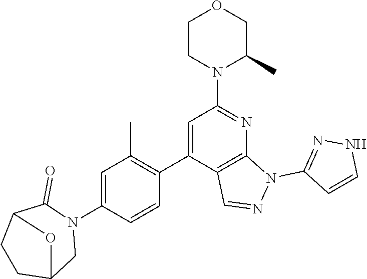 |
| 255 | 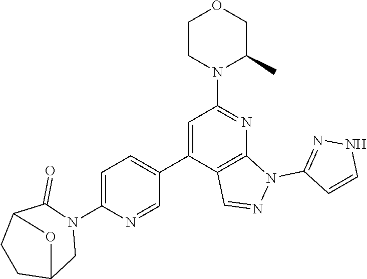 |
| 256 | 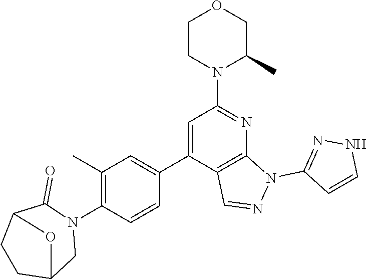 |
| 257 | 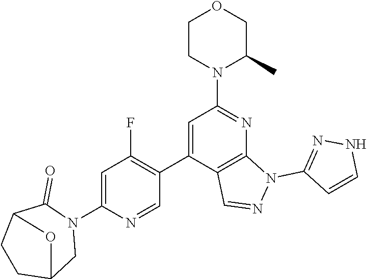 |
| 258 | 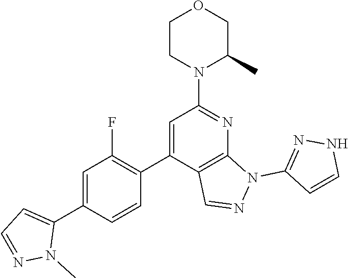 |
| 259 | 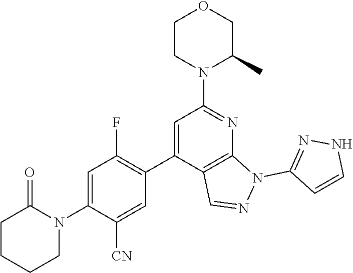 |
| 260 | 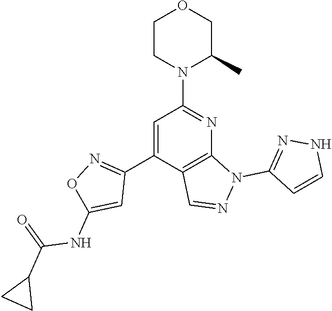 |
| 261 | 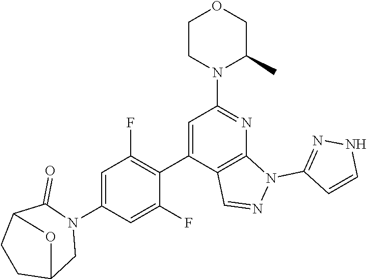 |
| 262 | 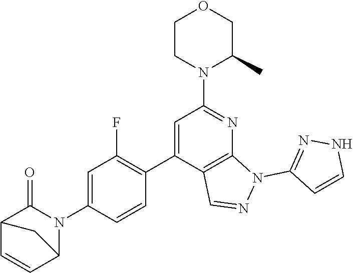 |
| 263 | 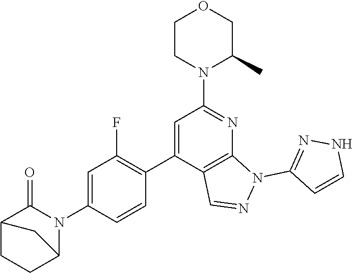 |
| 264 | 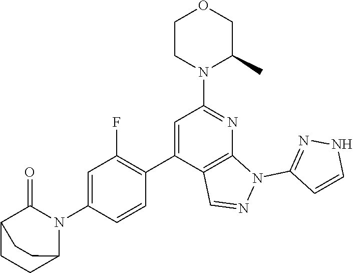 |
| 265 | 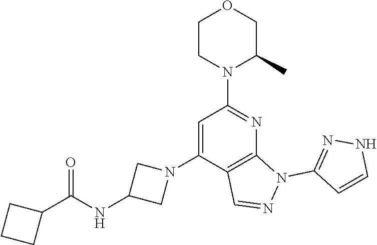 |
| 266 | 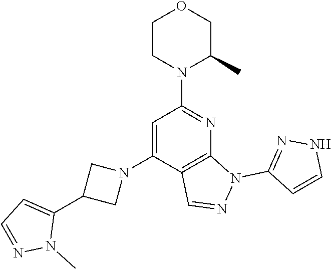 |
| 267 | 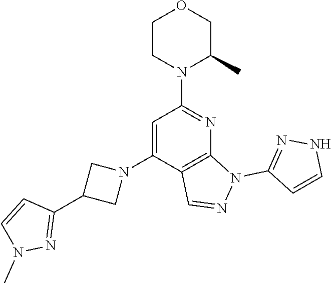 |
| 268 | 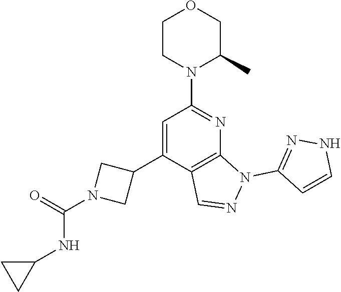 |
| 269 | 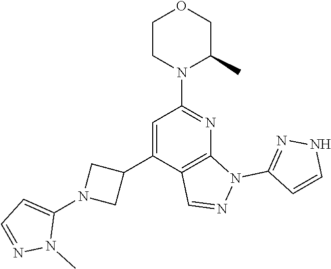 |
| 270 | 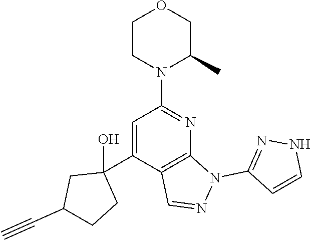 |
| 271 | 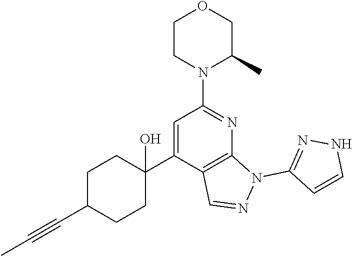 |
| 272 | 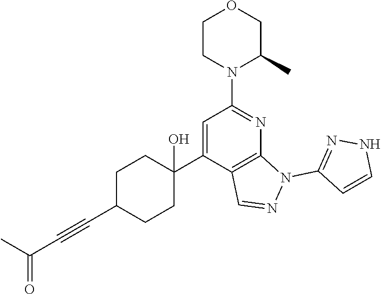 |
| 273 | 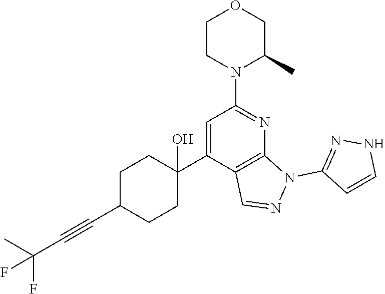 |
| 274 | 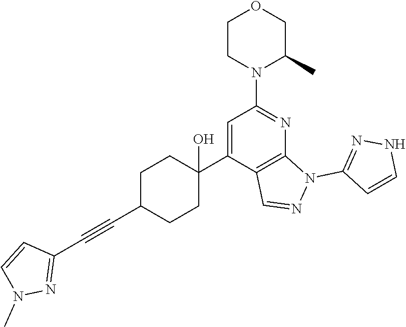 |
| 275 | 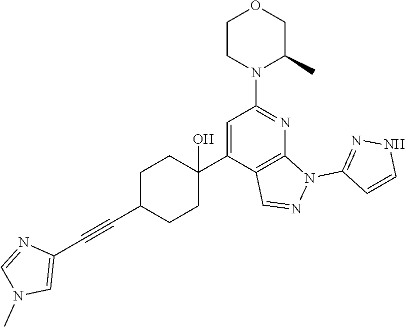 |
| 276 | 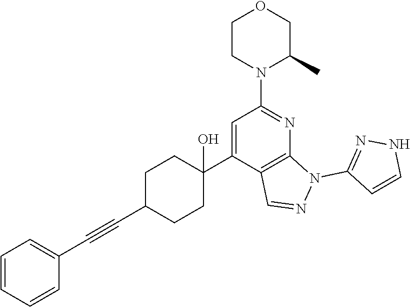 |
| 277 | 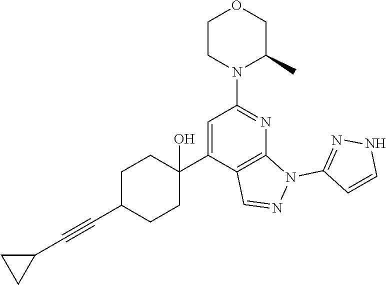 |
| 278 | 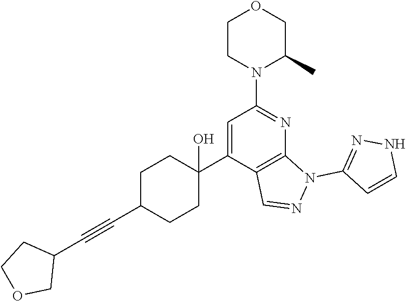 |
| 279 | 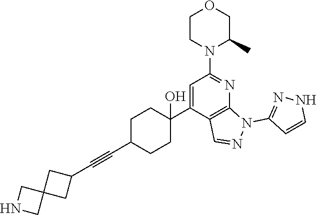 |
| 280 | 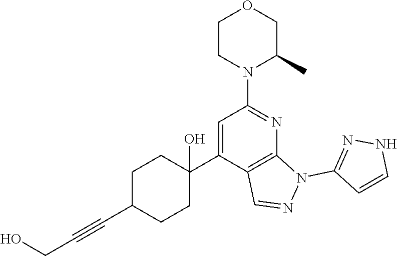 |
| 281 | 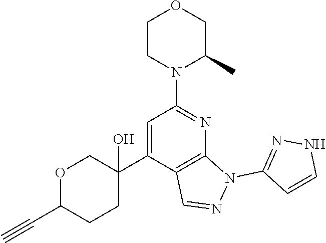 |
| 282 | 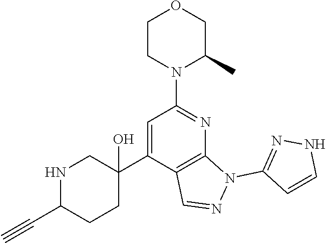 |
| 283 | 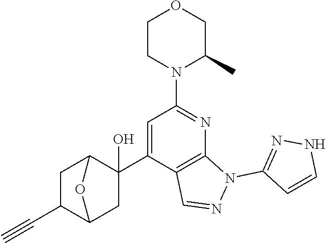 |
| 284 | 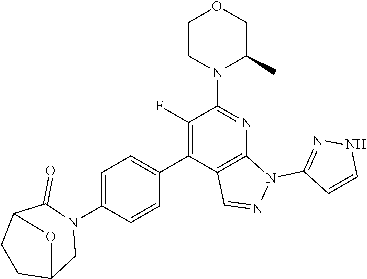 |
| 285 | 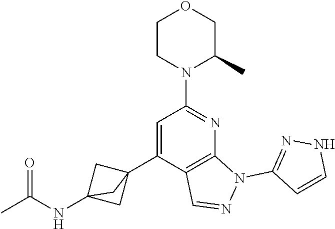 |
| 286 | 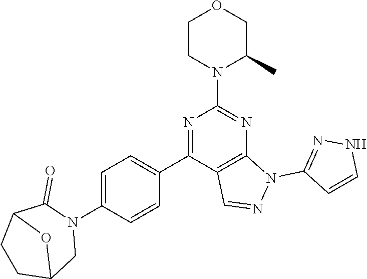 |
| 287 | 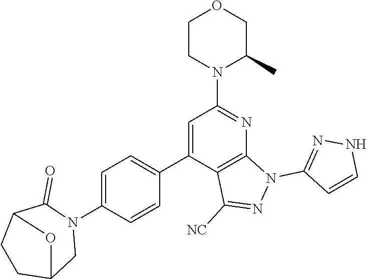 |
| 288 | 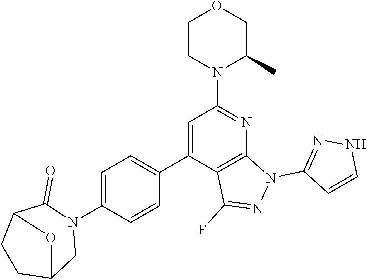 |
| 289 | 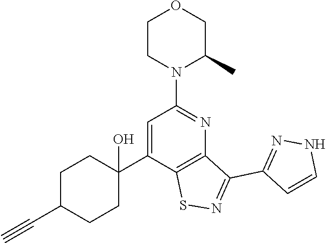 [SMILES: C#CC1CCC(O)(c2cc(N3CCOC[C@H]3C)nc3c(-c4cc[nH]n4)nsc23)CC1] |
| 290 | 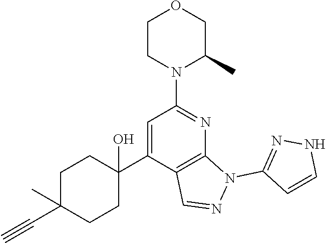 |
| 291 | 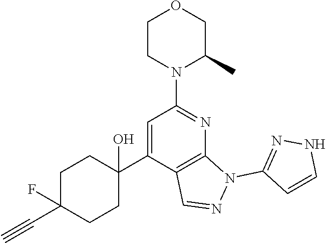 |
| 292 | 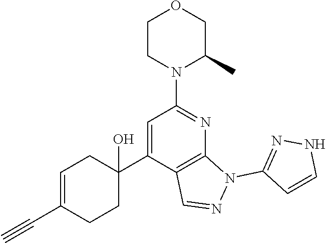 |
| 293 | 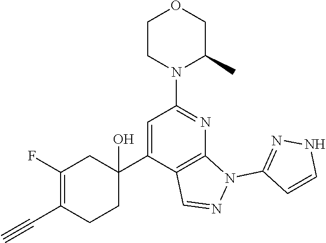 |

The present invention further provides a method for preparing the compound of the present invention, and the stereoisomer, the optical isomer, the pharmaceutical salt, the prodrug and the solvate thereof, wherein the method is selected from:

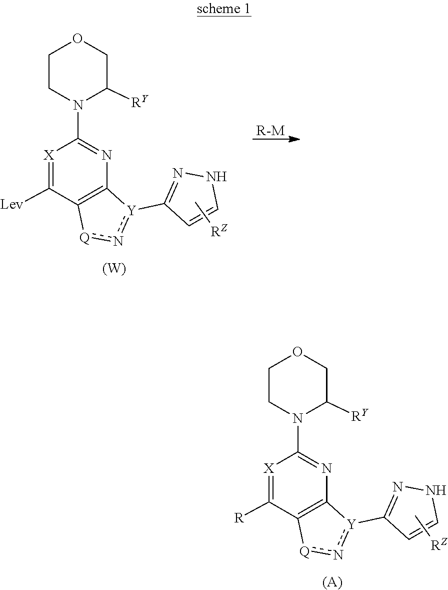

subjecting (W) and R-M to Suzuki cross-coupling reaction to obtain a compound as shown in formula (A);
wherein Lev is a leaving group selected from Cl, Br, I or OTf, more preferably I; M is a borate group or a boronic acid group; R is C₆₋₁₂ aryl, 5- to 12-membered heteroaryl, C₃₋₁₂ carbocyclyl or 3- to 12-membered heterocyclyl in the aforementioned formula (A), and other substituents are as defined previously;

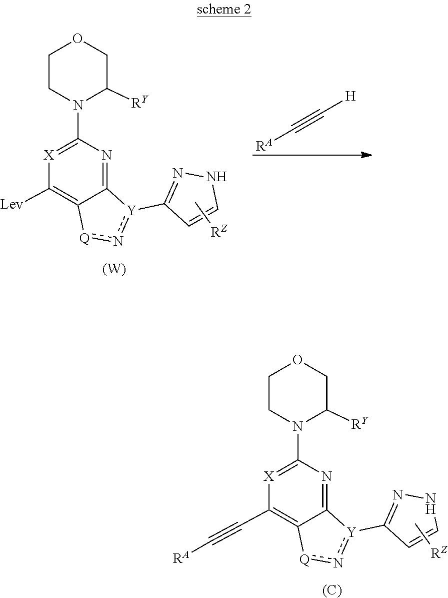

subjecting (W) and

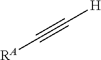

to Sonogashia cross-coupling reaction to obtain a compound as shown in formula (C);
wherein Lev is a leaving group selected from Cl, Br, I or OTf, more preferably I; other substituents are as defined previously.

The present invention further provides a pharmaceutical composition, comprising the compound of the present invention, and the stereoisomer, the optical isomer, the pharmaceutical salt, the prodrug and the solvate thereof.

The present invention further provides a pharmaceutical composition, comprising the compound of the present invention, and the stereoisomer, the optical isomer, the pharmaceutical salt, the prodrug and the solvate thereof, and a pharmaceutically acceptable excipient.

The objective of the present invention further includes providing the use of the compound of the present invention, and the stereoisomer, the optical isomer, the pharmaceutical salt, the prodrug and the solvate thereof, or the pharmaceutical composition of the present invention in the preparation of a drug for treating an ATR-mediated disease; preferably, the ATR-mediated disease is a cancer or tumor-related disease.

Further, the objective of the present invention further includes providing the use of the compound of the present invention, and the stereoisomer, the optical isomer, the pharmaceutical salt, the prodrug and the solvate thereof, or the pharmaceutical composition of the present invention in the preparation of a drug for treating a cancer or tumor-related disease; preferably, the cancer or tumor-related disease is a solid tumor; more preferably, the cancer or tumor-related disease is a digestive tract tumor; more preferably, the cancer or tumor-related disease is gastric cancer or colorectal cancer.

In some contexts of the art, the cancer may also be referred to as a tumor.

Regarding the use of the compound of the present invention, and the stereoisomer, the optical isomer, the pharmaceutical salt, the prodrug and the solvate thereof in the preparation of a drug for treating an ATR-mediated disease, the compound, and the stereoisomer, the optical isomer, the pharmaceutical salt, the prodrug and the solvate thereof are administered in combination with additional anti-cancer agents or immune checkpoint inhibitors for the treatment of cancers or tumors.

Regarding the use of the compound of the present invention, and the stereoisomer, the optical isomer, the pharmaceutical salt, the prodrug and the solvate thereof in the preparation of a drug for treating an ATR-mediated disease, the compound, and the stereoisomer, the optical isomer, the pharmaceutical salt, the prodrug and the solvate thereof are used in combination with additional treatments (e.g., radiotherapy) for the treatment of cancers or tumors.

The compound of the present invention, and the stereoisomer, the optical isomer, the pharmaceutical salt, the prodrug and the solvate thereof may provide enhanced anti-cancer effects when administered in combination with additional anti-cancer agents or immune checkpoint inhibitors for the treatment of cancers or tumors.

The objective of the present invention further includes providing a method for preventing and/or treating an ATR-mediated disease, comprising administering a therapeutically effective amount of the compound of the present invention, and the stereoisomer, the optical isomer, the pharmaceutical salt, the prodrug and the solvate thereof, or the pharmaceutical composition of the present invention to a patient. Further, the ATR-mediated disease according to the present invention is a cancer or tumor-related disease. Preferably, the ATR-mediated disease is a solid tumor; more preferably, the ATR-mediated disease is a digestive tract tumor; more preferably, the ATR-mediated disease is gastric cancer or colorectal cancer.

The objective of the present invention further includes providing a compound or a pharmaceutical composition comprising the compound, for use in the prevention and/or treatment of an ATR-mediated disease, wherein the compound is the compound of the present invention, and the stereoisomer, the optical isomer, the pharmaceutical salt, the prodrug and the solvate thereof. Further, the ATR-mediated disease according to the present invention is a cancer or tumor-related disease. Preferably, the ATR-mediated disease is a solid tumor; more preferably, the ATR-mediated disease is a digestive tract tumor; more preferably, the ATR-mediated disease is gastric cancer or colorectal cancer.

### Definitions

The term “optional”, “selectable”, “optionally” or “selectably” means that the subsequently described event or circumstance may, but not necessarily occur, and that the description includes instances where the event or circumstance occurs and instances where the event or circumstance does not occur.

The term “oxo” means that two hydrogen atoms at the same substitution site are replaced by the same oxygen atom to form a double bond.

Unless otherwise specified, the term “alkyl” refers to a monovalent saturated aliphatic hydrocarbon group, which is a linear or branched chain group containing 1 to 20 carbon atoms, preferably containing 1 to 10 carbon atoms (i.e., C₁₋₁₀ alkyl), further preferably containing 1 to 8 carbon atoms (C₁₋₈ alkyl), and more preferably containing 1 to 6 carbon atoms (i.e., C₁₋₆ alkyl). For example, “C₁₋₆ alkyl” means that the group is alkyl and the number of carbon atoms on the carbon chain is between 1 and 6 (specifically 1, 2, 3, 4, 5 or 6). Examples of alkyl include, but are not limited to methyl, ethyl, n-propyl, isopropyl, n-butyl, isobutyl, tert-butyl, sec-butyl, n-pentyl, neopentyl, 1,1-dimethylpropyl, 1,2-dimethylpropyl, 2,2-dimethylpropyl, 1-ethylpropyl, 2-methylbutyl, 3-methylbutyl, n-hexyl, n-heptyl, n-octyl, etc.

Unless otherwise specified, the term “alkenyl” refers to a linear or branched, unsaturated aliphatic hydrocarbon group consisting of carbon atoms and hydrogen atoms and having at least one double bond. Alkenyl may contain 2 to 20 carbon atoms, preferably contain 2 to 10 carbon atoms (i.e., C₂₋₁₀ alkenyl), further preferably contain 2 to 8 carbon atoms (C₂₋₈ alkenyl), and more preferably contain 2 to 6 carbon atoms (i.e., C₂₋₆ alkenyl), 2 to 5 carbon atoms (i.e., C₂₋₅ alkenyl), 2 to 4 carbon atoms (i.e., C₂₋₄ alkenyl), 2 to 3 carbon atoms (i.e., C₂₋₃ alkenyl) or 2 carbon atoms (i.e., C₂ alkenyl). For example, “C₂₋₆ alkenyl” means that the group is alkenyl and the number of carbon atoms on the carbon chain is between 2 and 6 (specifically 2, 3, 4, 5 or 6). Non-limiting examples of alkenyl include, but are not limited to ethenyl, 1-propenyl, 2-propenyl, 1-butenyl, isobutenyl, 1,3-butadienyl, etc.

Unless otherwise specified, the term “alkynyl” refers to a linear or branched, unsaturated aliphatic hydrocarbon group consisting of carbon atoms and hydrogen atoms and having at least one triple bond. Alkynyl may contain 2 to 20 carbon atoms, preferably contain 2 to 10 carbon atoms (i.e., C₂₋₁₀ alkynyl), further preferably contain 2 to 8 carbon atoms (C₂₋₈ alkynyl), and more preferably contain 2 to 6 carbon atoms (i.e., C₂₋₆ alkynyl), 2 to 5 carbon atoms (i.e., C₂₋₅ alkynyl), 2 to 4 carbon atoms (i.e., C₂₋₄ alkynyl), 2 to 3 carbon atoms (i.e., C₂₋₃ alkynyl) or 2 carbon atoms (i.e., C₂ alkynyl). For example, “C₂₋₆ alkynyl” means that the group is alkynyl and the number of carbon atoms on the carbon chain is between 2 and 6 (specifically 2, 3, 4, 5 or 6). Non-limiting examples of alkynyl include, but are not limited to ethynyl, 1-propynyl, 2-propynyl, 1-butynyl, etc.

Unless otherwise specified, the term “cycloalkyl” refers to a monocyclic or polycyclic saturated aliphatic hydrocarbon group with a specific number of carbon atoms, preferably containing 3 to 12 carbon atoms (i.e., C₃₋₁₂ cycloalkyl), more preferably containing 3 to 10 carbon atoms (C₃₋₁₀ cycloalkyl), and further preferably containing 3 to 6 carbon atoms (C₃₋₆ cycloalkyl), 4 to 6 carbon atoms (C₄₋₆ cycloalkyl) or 5 to 6 carbon atoms (C₅₋₆ cycloalkyl). Examples of cycloalkyl include, but are not limited to cyclopropyl, cyclobutyl, cyclopentyl, cyclohexyl, methylcyclopropyl, 2-ethyl-cyclopentyl, dimethylcyclobutyl, etc.

Unless otherwise specified, the term “alkoxy” refers to —O-alkyl, the alkyl being defined as above as containing 1 to 20 carbon atoms, preferably containing 1 to 10 carbon atoms, preferably 1 to 8 carbon atoms, and more preferably 1 to 6 carbon atoms (specifically 1, 2, 3, 4, 5 or 6). Representative examples of alkoxy include, but are not limited to methoxy, ethoxy, propoxy, isopropoxy, butoxy, 1-methylpropoxy, 2-methylpropoxy, tert-butoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, etc.

Unless otherwise specified, the term “halogen” or “halo” refers to F, Cl, Br and I. The term “haloalkyl” means an alkyl group as defined above in which one, two or more hydrogen atoms or all hydrogen atoms have been replaced by halogen. Representative examples of haloalkyl include CCl₃, CF₃, CHCl₂, CH₂Cl, CH₂Br, CH₂I, CH₂CF₃, CF₂CF₃, etc.

Unless otherwise specified, the term “heterocyclyl” refers to a saturated or partially unsaturated monocyclic, bicyclic or polycyclic hydrocarbon substituent that is non-aromatic, including those with some rings in aromatic structure for a polycyclic system. Heterocyclyl contains 3 to 20 ring atoms, wherein 1, 2, 3 or more ring atoms are selected from N, O or S, and the remaining ring atoms are C. Heterocyclyl preferably contains 3 to 12 ring atoms, further preferably contains 3 to 10 ring atoms, or 3 to 8 ring atoms, or 3 to 6 ring atoms, or 4 to 6 ring atoms, or 5 to 6 ring atoms. The number of heteroatoms is preferably from 1 to 4, more preferably from 1 to 3 (i.e., 1, 2 or 3). Examples of monocyclic heterocyclyl include pyrrolidyl, imidazolidinyl, tetrahydrofuryl, dihydropyrrolyl, piperidyl, piperazinyl, pyranyl, etc. Polycyclic heterocyclyl includes spiro, fused and bridged heterocyclyl.

Unless otherwise specified, “heterocycloalkyl” refers to saturated “heterocyclyl” or “heterocycle” as defined above, with ring atoms as defined above, i.e., containing 3 to 20 ring atoms (“3- to 20-membered heterocycloalkyl”), and the number of heteroatoms being from 1 to 4 (1, 2, 3 or 4), preferably from 1 to 3 (1, 2 or 3), wherein the heteroatoms are each independently selected from N, O or S. Heterocycloalkyl preferably contains 3 to 14 ring atoms (“3- to 14-membered heterocycloalkyl”), further preferably contains 3 to 10 ring atoms (“3- to 10-membered heterocycloalkyl”), still further preferably contains 3 to 8 ring atoms (“3- to 8-membered heterocycloalkyl”), still further preferably contains 4 to 7 ring atoms (“4- to 7-membered heterocycloalkyl”), still further preferably contains 5 to 10 ring atoms (“5- to 10-membered heterocycloalkyl”), and still further preferably contains 5 to 6 ring atoms (“5- to 6-membered heterocycloalkyl”). In some embodiments, each example of heterocycloalkyl is independently optionally substituted, e.g., unsubstituted (“unsubstituted heterocycloalkyl”) or substituted with one or more substituents (“substituted heterocycloalkyl”). Some examples of “heterocycloalkyl” are listed in the “heterocyclyl” or “heterocycle” section above, and include, but are not limited to aziridinyl, oxiranyl, thiiranyl, azetidinyl, oxetanyl, thietanyl, tetrahydrofuryl, oxanyl, piperidyl, piperazinyl, morpholinyl, thiomorpholinyl, oxathianyl, oxazolidinyl, dioxanyl, dithianyl, thiazolidinyl, pyrrolidyl, pyrazolidinyl, imidazolidine, etc.

Unless otherwise specified, the term “carbocyclyl” or “carbocycle” refers to a non-aromatic cyclic hydrocarbon group having ring carbon atoms ranging from 3 to 14 (“C₃₋₁₄ carbocyclyl”) and not having heteroatoms in the non-aromatic ring system. In some embodiments, the carbocyclyl group has 3 to 12 ring carbon atoms (“C₃₋₁₂ carbocyclyl”), or 4 to 12 ring carbon atoms (“C₄₋₁₂ carbocyclyl”), or 3 to 10 ring carbon atoms (“C₃₋₁₀ carbocyclyl”). In some embodiments, the carbocyclyl group has 3 to 8 ring carbon atoms (“C₃₋₈ carbocyclyl”). In some embodiments, the carbocyclyl group has 3 to 7 ring carbon atoms (“C₃₋₇ carbocyclyl”). In some embodiments, the carbocyclyl group has 4 to 6 ring carbon atoms (“C₄₋₆ carbocyclyl”). In some embodiments, the carbocyclyl group has 5 to 10 ring carbon atoms (“C₅₋₁₀ carbocyclyl”), or 5 to 7 ring carbon atoms (“C₅₋₇ carbocyclyl”). Exemplary C₃₋₆ carbocyclyl groups include, but are not limited to cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), etc. Exemplary C₃₋₈ carbocyclyl groups include, but are not limited to C₃₋₆ carbocyclyl groups as mentioned above, cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), etc. Exemplary C₃₋₁₀ carbocyclyl groups include, but are not limited to C₃₋₈ carbocyclyl groups as mentioned above, cyclononyl (C₉), cyclononenyl (C₉), cyclodecyl (C₁₀), cyclodecenyl (C₁₀), octahydro-1H-indenyl (C₉), decahydronaphthyl (C₁₀), spiro[4.5]decanyl (C₁₀), etc. As illustrated in the above examples, in some embodiments, the carbocyclyl group is monocyclic (“monocyclic carbocyclyl”), or is a fused (fused ring group), bridged (bridged ring group), or spiro-fused (spiro ring group) cyclic system, such as a bicyclic system (“bicyclic carbocyclyl”), and may be saturated or may be partially unsaturated. “Carbocyclyl” further includes a cyclic system in which the carbocyclic ring as defined above is fused with one or more aryl or heteroaryl groups, wherein the point of attachment is on the carbocyclic ring, and wherein in such cases the number of carbons continues to be indicative of the number of carbons in the carbocyclic system. In some embodiments, each example of carbocyclyl groups is independently optionally substituted, e.g., unsubstituted (“unsubstituted carbocyclyl”) or substituted with one or more substituents (“substituted carbocyclyl”). In some embodiments, the carbocyclyl group is unsubstituted C₃₋₁₀ carbocyclyl. In some embodiments, the carbocyclyl group is substituted C₃₋₁₀ carbocyclyl.

Unless otherwise specified, “cycloalkenyl” refers to a group consisting of subgroups monocyclic hydrocarbon ring, bicyclic hydrocarbon ring and spiro hydrocarbon ring; however, the system is unsaturated, i.e., there is at least one C═C double bond, but no aromatic system. Cycloalkenyl preferably contains 3 to 12 carbon atoms (i.e., C₃₋₁₂ cycloalkenyl), more preferably contains 3 to 10 carbon atoms (C₃₋₁₀ cycloalkenyl), and further preferably 3 to 6 carbon atoms (C₃₋₆ cycloalkenyl), 4 to 6 carbon atoms (C₄₋₆ cycloalkenyl) or 5 to 6 carbon atoms (C₅₋₆ cycloalkenyl).

Unless otherwise specified, the term “fused ring” refers to a non-aromatic saturated or partially unsaturated bicyclic or polycyclic system formed by two or more cyclic structures sharing two neighboring atoms with each other, including fused carbocyclyl and fused heterocyclyl, wherein the “fused heterocyclyl” optionally contains one or more heteroatoms independently selected from oxygen, nitrogen and sulfur.

Unless otherwise specified, the term “spirocycloalkyl” refers to a saturated ring system with a specific number of carbon atoms consisting of carbon and hydrogen atoms sharing only one ring carbon atom. The spirocycloalkyl is preferably 6- to 14-membered, and more preferably 7- to 10-membered. Non-limiting examples of monospiro ring groups include 3-membered/5-membered, 4-membered/4-membered, 4-membered/5-membered, 4-membered/6-membered, 5-membered/5-membered and 5-membered/6-membered monospiro ring groups, wherein the number of ring atoms in each case includes the spiro atoms. Non-limiting examples of monospiro ring groups include:

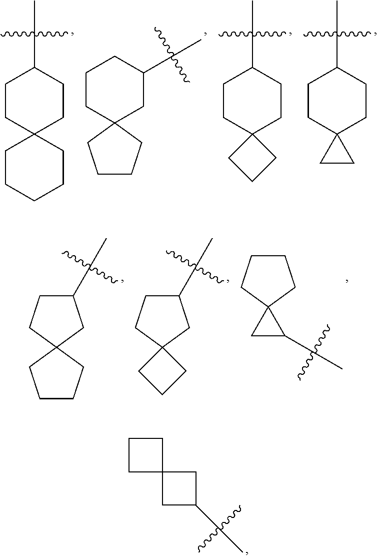

etc.

Unless otherwise specified, the term “heterospiro ring group” or “spiro heterocyclyl” refers to a cyclic structure with a specific number of carbon atoms and heteroatoms formed by two or more saturated rings sharing one ring carbon atom. The number of heteroatoms in the spiro heterocyclyl is preferably from 1 to 4, and more preferably from 1 to 3 (i.e., 1, 2 or 3), and the heteroatoms are independently selected from N, O and S. The spiro heterocyclyl is preferably 6- to 14-membered, and more preferably 7- to 10-membered. Non-limiting examples of spiro heterocyclyl include 3-membered/5-membered, 4-membered/4-membered, 4-membered/5-membered, 4-membered/6-membered, 5-membered/5-membered and 5-membered/6-membered spiro heterocyclyl groups, wherein the number of rings in each case includes the spiro atoms. Non-limiting examples of hetero-monospiro ring groups include:

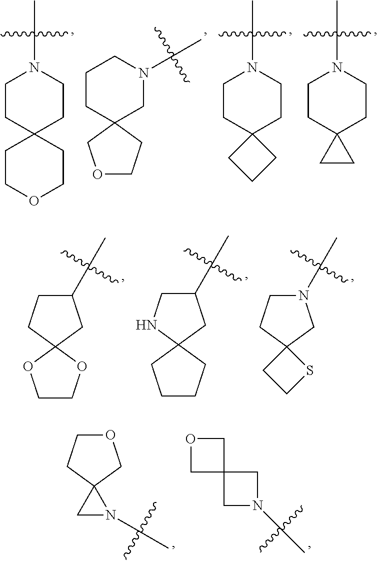

, etc.

Unless otherwise specified, the term “bridged ring group” refers to a 5- to 20-membered all-carbon polycyclic group with any two rings sharing two carbon atoms that are not directly connected. It may contain one or more double bonds, but none of the rings has a fully conjugated n electron system. The bridged ring group is preferably 6- to 14-membered, and more preferably 7- to 10-membered. According to the number of constituent rings, it can be divided into bicyclic, tricyclic, tetracyclic or polycyclic bridged cycloalkyl, preferably bicyclic, tricyclic or tetracyclic, and more preferably bicyclic or tricyclic. Non-limiting examples of bridged cycloalkyl include:

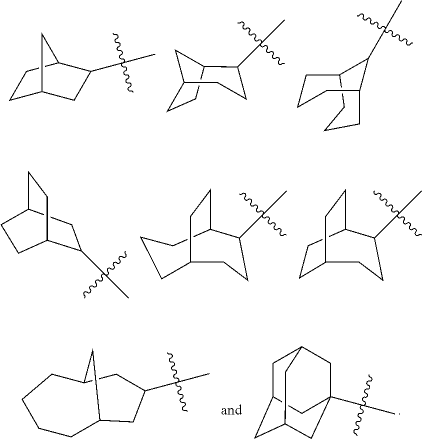

Unless otherwise specified, the term “heterobridged ring group” or “bridged heterocyclyl” refers to 5- to 14-membered polycyclic heterocyclic group with any two rings sharing two ring atoms that are not directly connected. It may contain one or more double bonds, but none of the rings has a fully conjugated π electron system. The number of heteroatoms in the bridged heterocyclyl is one or more, preferably from 1 to 4, and more preferably from 1 to 3 (i.e., 1, 2 or 3), and the heteroatoms are independently selected from N, O or S(O)m (wherein m is an integer from 0 to 2), with the remaining ring atoms being carbon. The bridged heterocyclyl is preferably 6- to 14-membered, and more preferably 7- to 10-membered. According to the number of constituent rings, it can be divided into bicyclic, tricyclic, tetracyclic or polycyclic bridged heterocyclyl, preferably bicyclic, tricyclic or tetracyclic, and more preferably bicyclic or tricyclic. Non-limiting examples of bridged heterocyclyl include:

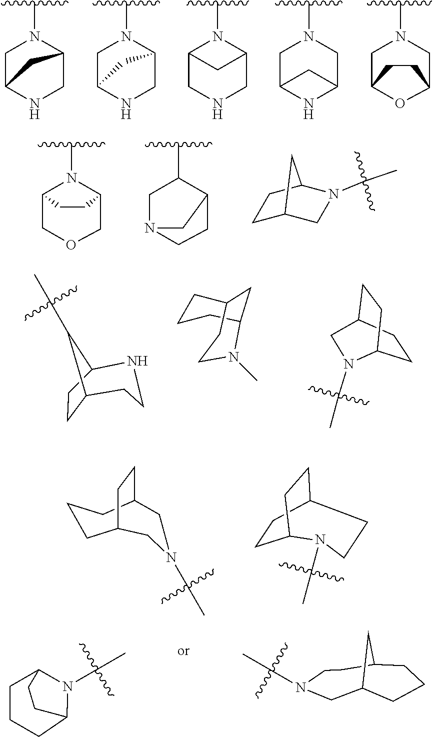

etc.

Unless otherwise specified, the term “aryl” refers to monocyclic, bicyclic and tricyclic aromatic carbocyclic systems containing 6 to 16 carbon atoms, or 6 to 14 carbon atoms, or 6 to 12 carbon atoms, or 6 to 10 carbon atoms, preferably 6 to 10 carbon atoms. The term “aryl” can be used interchangeably with the term “aromatic ring group”. Examples of aryl groups may include, but are not limited to phenyl, naphthyl, anthryl, phenanthryl, pyrenyl, etc.

Unless otherwise specified, the term “heteroaryl” refers to an aromatic monocyclic or polycyclic system containing a 5- to 12-membered structure, or preferably a 5- to 10-membered structure or a 5- to 8-membered structure, and more preferably a 5- to 6-membered structure, wherein 1, 2, 3 or more ring atoms are heteroatoms and the remaining atoms are carbon, the heteroatoms are independently selected from O, N or S, and the number of heteroatoms is preferably 1, 2 or 3. Examples of heteroaryl include, but are not limited to furyl, thienyl, oxazolyl, thiazolyl, isoxazolyl, oxadiazolyl, thiadiazolyl, pyrrolyl, pyrazolyl, imidazolyl, triazolyl, tetrazolyl, pyridyl, pyrimidyl, pyrazinyl, pyridazinyl, thiodiazolyl, triazinyl, phthalazinyl, quinolyl, isoquinolyl, pteridinyl, purinyl, indolyl, isoindolyl, indazolyl, benzofuryl, benzothienyl, benzopyridyl, benzopyrimidyl, benzopyrazinyl, benzoimidazolyl, benzophthalazinyl, pyrrolo[2,3-b]pyridyl, imidazo[1,2-a]pyridyl, pyrazolo[1,5-a]pyridyl, pyrazolo[1,5-a]pyrimidyl, imidazo[1,2-b]pyridazinyl, [1,2,4]triazolo[4,3-b]pyridazinyl, [1,2,4]triazolo[1,5-a]pyrimidyl, [1,2,4]triazolo[1,5-a]pyridyl, etc.

Unless otherwise specified, the term “pharmaceutically acceptable salt”, “pharmaceutical salt” or “medicinal salt” refers to salts that are, within the scope of sound medical judgment, suitable for contact with mammalian tissues, particularly human tissues without excessive toxicity, irritation, allergic response, etc., and that are commensurate with a reasonable benefit/risk ratio. The salts may be prepared in situ during the final isolation and purification of the compound of the present invention, or separately by reacting the free base or free acid with a suitable reagent.

Unless otherwise specified, the term “solvate” means a physical association of the compound of the present invention with one or more solvent molecules, whether organic or inorganic. This physical association involves hydrogen bonding. In certain cases, the solvate can be isolated, for example when one or more solvent molecules are incorporated in the crystal lattice of the crystalline solid. The solvent molecules in a solvate may exist in a regular and/or disordered arrangement. Solvates may include stoichiometric or non-stoichiometric amounts of solvent molecules. “Solvate” encompasses both solution-phase and isolatable solvates. Exemplary solvates include, but are not limited to hydrates, ethanolates, methanolates, and isopropanolates. Solvation methods are well known in the art.

Unless otherwise specified, the term “isotopically labeled analog” or “isotopic derivative” refers to molecules of the compounds of formula I to formula II that are isotopically labeled, thereby providing isotopically labeled analogs that may have improved pharmacological activities. The isotopes commonly used for isotopic labeling are: hydrogen isotopes ²H and ³H; carbon isotopes ¹¹C, ¹³C and ¹⁴C; chlorine isotopes ³⁵Cl and ³⁷Cl; fluorine isotope ¹⁸F; iodine isotopes ¹²³I and ¹²⁵I; nitrogen isotopes ¹³N and ¹⁵N; oxygen isotopes ¹⁵O, ¹⁷O and ¹⁸O, and sulfur isotope ³⁵S. These isotopically labeled compounds can be used to study the distribution of pharmaceutical molecules in tissues. Particularly, deuterium ³H and carbon ¹³C are more widely used because they are easy to label and convenient to detect. Substitution with certain heavy isotopes, such as deuterium (²H), can enhance metabolic stability and prolong half-life, thereby achieving the goal of reducing dosage and providing therapeutic advantages. The synthesis of isotopically labeled compounds is generally performed in the same manner as non-isotopically labeled compounds, starting from labeled starting materials using known synthetic techniques. Generally, the compound of the present invention includes isotopic derivatives thereof (such as deuterated compounds).

Unless otherwise specified, the term “optical isomer” refers to substances that have exactly the same molecular structure, similar physical and chemical properties, but different optical rotations.

Unless otherwise specified, the term “stereoisomer” refers to compounds that have identical chemical constitutions, but differ in the way the atoms or groups are arranged in space. Stereoisomers include enantiomers, diastereomers, conformational isomers (rotamers), geometric isomers (cis/trans isomers), atropisomers, etc. Any resulting mixture of stereoisomers may be separated into pure or substantially pure geometric isomers, enantiomers, or diastereomers based on differences in the physical and chemical properties of the components, e.g., by chromatography and/or fractional crystallization.

Unless otherwise specified, the term “tautomer” refers to structural isomers of different energies that are interconvertible via a low energy barrier. Where tautomerization is possible (e.g., in solution), a chemical equilibrium of tautomers can be achieved. For example, proton tautomers (also known as prototropic tautomers) include interconversions via migration of a proton, such as keto-enol and imine-enamine isomerizations. Valence tautomers involve interconversions by rearrangement of some of the bonding electrons.

Unless otherwise indicated, the structural formula described in the present invention includes all isomeric forms (such as enantiomeric, diastereomeric, and geometric isomeric (or conformational isomeric) forms), such as R and S configurations containing asymmetric centers, (Z) and (E) isomers of double bonds, and (Z) and (E) conformational isomers. Therefore, a single stereochemical isomer of the compound of the present invention, or mixtures of enantiomers, diastereomers, or geometric isomers (or conformational isomers) thereof are within the scope of the present invention.

Unless otherwise specified, the term “prodrug” refers to a drug that is converted in vivo to the parent drug. Prodrugs are often useful because, in some situations, they may be easier to administer than the parent drug. For example, they may be bioavailable through oral administration, whereas the parent is not. The solubility of the prodrug in pharmaceutical compositions is also improved compared with the parent drug. An example of a prodrug may include, but is not limited to, any compound of formula I which is administered as an ester (“prodrug”) to facilitate the delivery across the cell membrane where water solubility is detrimental to mobility, the prodrug is then metabolically hydrolyzed to the carboxylic acid, the active entity, once inside the cell where water solubility is beneficial. Another example of a prodrug may be a short peptide (polyamino acid) bound to an acid group, wherein the peptide is metabolized to provide the active moiety.

Unless otherwise specified, the term “optionally substituted” means that the hydrogen at the substitution site of the group is unsubstituted, or is substituted with one or more substituents preferably selected from the following group: halogen, hydroxyl, sulfhydryl, cyano, nitro, amino, azide group, oxo, carboxyl, C₂₋₆ alkenyl, C₂₋₆ alkynyl, C₁₋₆ alkyl, C₁₋₆ alkoxy, C₃₋₁₀ cycloalkyl, C₃₋₁₀ cycloalkylsulfonyl, 3- to 10-membered heterocycloalkyl, C₆₋₁₄ aryl or 5- to 10-membered heteroaromatic ring group, wherein the C₂₋₆ alkenyl, C₂₋₆ alkynyl, C₁₋₆ alkyl, C₁₋₆ alkoxy, C₃₋₁₀ cycloalkyl, C₃₋₁₀ cycloalkylsulfonyl, 3- to 10-membered heterocycloalkyl, C₆₋₁₄ aryl or 5- to 10-membered heteroaromatic ring group may be optionally substituted with one or more substituents selected from halogen, hydroxyl, amino, cyano, C₁₋₆ alkyl or C₁₋₆ alkoxy, and the oxo means that two H at the same substitution site are replaced by the same O to form a double bond.

The beneficial effects of the present invention are as follows.

The present invention designs a class of compounds with novel structures, providing a new direction for the development of ATR inhibitor drugs. An in vitro enzyme inhibitory activity study shows that the compounds of the present invention have a strong inhibitory effect on an ATR enzyme; an in vitro experimental study regarding the inhibitory effect on cell proliferation shows that the compounds of the present invention have a significant inhibitory effect on the proliferation of both LoVo cells and SNU-601 cells; therefore, the compounds of the present invention may serve as promising compounds for the treatment of ATR-mediated diseases. In addition, the present invention explores a specific synthesis method, which is simple in process, convenient in operation, and conducive to large-scale industrial production and application.

### DETAILED DESCRIPTION OF EMBODIMENTS

The present invention is further described below in conjunction with specific examples. It should be understood that these examples are merely used for describing the present invention, rather than limiting the scope of the present invention. In the following examples, conventional conditions or conditions suggested by the manufacturers are generally used, unless specific conditions indicated in an experimental method. Unless otherwise defined, all professional and scientific terms used herein have the same meanings as those commonly understood by a person skilled in the art. In addition, any methods and materials similar or equivalent to the content described herein can all be applied in the method of the present invention. The preferred embodiments and materials described herein are meant for exemplary purposes only.

The structure of the compound of the present invention is determined by nuclear magnetic resonance (NMR) or/and liquid chromatography-mass spectrometry (LC-MS) or/and high performance liquid chromatography (HPLC). The instrument used for NMR determination is the Agilent 400/54 Premium Shielded NMR Magnet System; the instrument used for LC-MS is the Shimadzu LCMS2020; the instrument used for HPLC is the Agilent 1200.

The starting materials in the examples of the present invention are known and commercially available, or can be synthesized by or in accordance with the methods known in the art.

### Description of Terms or Abbreviations

LDA: Lithium diisopropylamide
NMP: N-methylpyrrolidone
SEMCI: [2-(Chloromethoxy)ethyl]trimethylsilane
THF: Tetrahydrofuran
DMSO: Dimethyl sulfoxide
Pd(dppf)Cl₂: [1,1′-Bis(diphenylphosphino)ferrocene]dichloropalladium (II)
DCM: Dichloromethane
TFA: Trifluoroacetic acid
Et₃SiH: Triethylsilane
mCPBA: m-Chloroperoxybenzoic acid
Xphos: Dicyclohexyl[2′,4′,6′-tri(propan-2-yl)-[1,1′-biphenyl]-2-yl]phosphine
n-BuLi: n-Butyllithium
TBAF: Tetrabutylammonium fluoride
DIEA: N,N-Diisopropylethylamine
PdCl₂(PPh₃)₂: Bis(triphenylphosphine)dichloropalladium (II)
B₂Pin₂: Bis(pinacolato)diboron
HATU: 2-(7-Azabenzotriazole)-N,N,N′,N′-tetramethyluronium hexafluorophosphate
TBDPSCl: Tert-butyl diphenylsilyl chloride
LiHMDS: Lithium hexamethyldisilazide
TMSI: Trimethyliodosilane Example 1

### Preparation of (3R)-3-methyl-4-(4-(5-methyl-3-(trifluoromethyl)-1H-pyrazolyl-4-yl)-1-(1H-pyrazolyl-3-yl)-1H-pyrazolyl[3,4-b]pyridin-6-yl)morpholine

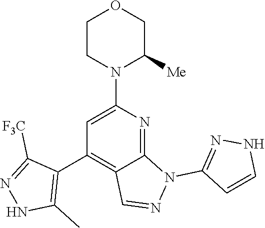

### Step 1: Preparation of 5-hydrazinyl-1H-pyrazole

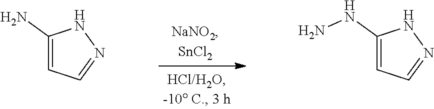 [SMILES: Nc1ccn[nH]1 ; NNc1ccn[nH]1]

5-Amino-1H-pyrazole (16 g, 192.56 mmol, 1 equiv.) was weighed, 6 M aqueous hydrogen chloride solution (320 mL, 1.92 mol, 10 equiv.) was added, and the reaction liquid was cooled to −10° C. with stirring. Sodium nitrite (13.29 g, 192.56 mmol, 1 equiv.) dissolved in water (200 ml) was slowly added dropwise to the reaction liquid, and the mixture was stirred for additional 1 hour. Stannous chloride (72.6 g, 383.2 mmol, 1.99 equiv.) dissolved in 6 M aqueous hydrogen chloride solution (80 mL) was slowly added dropwise to the reaction liquid, and the mixture was stirred for additional 2 hours. After the reaction was completed as monitored by TLC, the reaction liquid was concentrated, and the residue was purified by reverse-phase column chromatography (C18, acetonitrile:water=0%-5%), to afford the target compound (32 g, yield: 100%). ¹H NMR (400 MHz, DMSO-d6) δ 7.58 (d, J=2.3 Hz, 1H), 5.78 (d, J=2.2 Hz, 1H).

### Step 2: Preparation of 2,6-difluoro-4-iodonicotinaldehyde

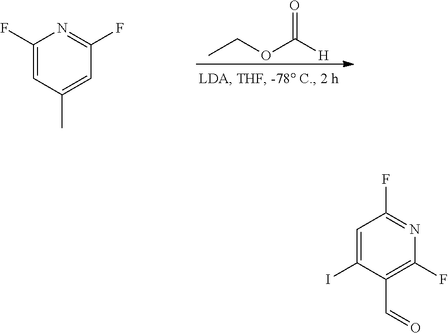

2,6-Difluoro-4-iodopyridine (14 g, 58.1 mmol, 1 equiv.) was dissolved in tetrahydrofuran (150 mL), and the mixture was cooled to −78° C. Lithium diisopropylamide (34.85 mL, 325.34 mmol, 5.6 equiv.) was added dropwise and the mixture was stirred for additional 1 hour, and then ethyl formate (6.46 g, 87.14 mmol, 1.5 equiv.) was added and the resulting mixture was stirred for additional half an hour. After the reaction was completed as monitored by TLC, the reaction was quenched with saturated ammonium chloride solution, and the reaction liquid was stirred for additional 10 minutes and extracted with dichloromethane (300 mL) and water (300 mL). The organic phase was dried, filtered and concentrated, and the residue was purified by column chromatography (ethyl acetate:petroleum ether=0%-5%), to afford the target compound (5 g, yield: 32%). ¹H NMR (400 MHz, DMSO-d6) δ 9.93 (s, 1H), 7.95 (d, J=2.3 Hz, 1H).

### Step 3: Preparation of (E)-3-((2-(1H-pyrazol-5-yl)hydrazone)methyl)-2,6-difluoro-4-iodopyridine

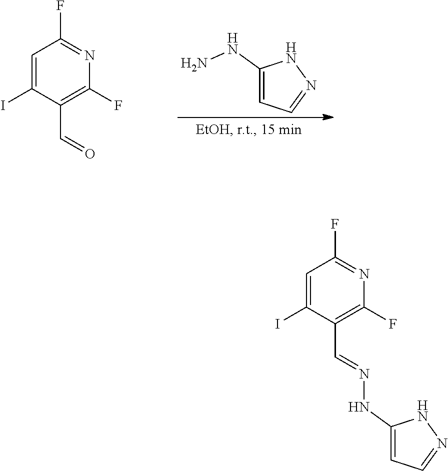 [SMILES: O=Cc1c(I)cc(F)nc1F ; NNc1ccn[nH]1 ; Fc1cc(I)c(/C=N/Nc2ccn[nH]2)c(F)n1]

2,6-Difluoro-4-iodonicotinaldehyde (7 g, 26.02 mmol, 1 equiv.) and 5-hydrazinyl-1H-pyrazole (28.05 g, 286.22 mmol, 11 equiv.) were dissolved in ethanol (500 mL), and the reaction liquid was stirred at room temperature for 15 minutes. After the reaction was completed as monitored by LCMS, the reaction liquid was concentrated and filtered with diatomaceous earth, the filter cake was washed with a small amount of methanol, and the filtrate was purified by reverse-phase column chromatography (C18, acetonitrile:water=0%-50%), to afford the target compound (5 g, yield: 55.07%). LCMS (ESI) [M+H]+=349.90.

### Step 4: Preparation of 6-fluoro-4-iodo-1-(1H-pyrazol-5-yl)-1H-pyrazole[3,4-b]pyridine

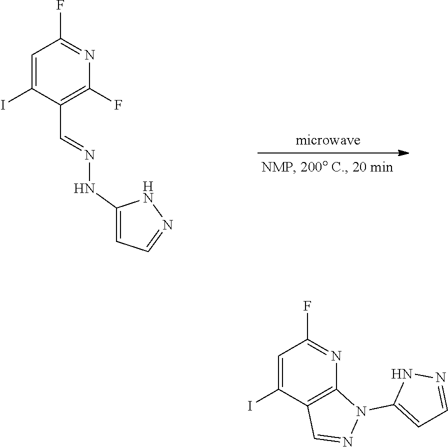 [SMILES: Fc1cc(I)c(/C=N/Nc2ccn[nH]2)c(F)n1 ; Fc1cc(I)c2cnn(-c3ccn[nH]3)c2n1]

(E)-3-((2-(1H-Pyrazol-5-yl)hydrazone)methyl)-2,6-difluoro-4-iodopyridine (1 g, 2.86 mmol, 1 equiv.) dissolved in N-methylpyrrolidone (15 mL) was placed in a microwave tube, and the reaction liquid was heated to 200° C. under microwave and stirred for 20 minutes. After the reaction was completed as monitored by LCMS, the reaction liquid was extracted with ethyl acetate (30 mL) and water (30 mL), the organic phase was dried, filtered and concentrated, and the residue was purified by column chromatography (ethyl acetate:petroleum ether=1:1), to afford the target compound (320 mg, yield: 33.97%). LCMS (ESI) [M+H]+=329.85.

### Step 5: Preparation of (R)-4-(4-iodo-1-(1H-pyrazol-5-yl)-1H-pyrazole[3,4-b]pyridin-6-yl)-3-methylmorpholine

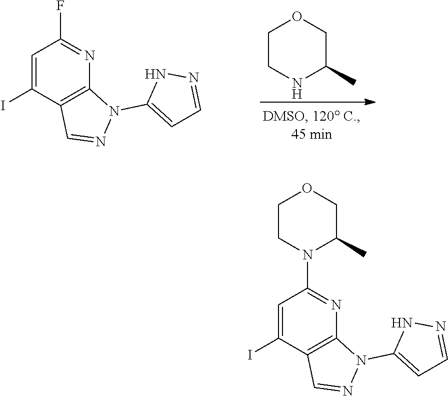

6-Fluoro-4-iodo-1-(1H-pyrazol-5-yl)-1H-pyrazole[3,4-b]pyridine (319.2 mg, 0.97 mmol, 1 equiv.) dissolved in DMSO (5 mL) was placed in a sealed tube, (R)-3-methylmorpholine (117.74 mg, 1.16 mmol, 1.2 equiv.) was added, and the reaction liquid was heated to 120° C. and stirred for 1 hour. After the reaction was completed as monitored by LCMS, the reaction liquid was extracted with water (10 mL) and ethyl acetate (10 mL), the organic phase was concentrated, and the residue was purified by thin layer chromatography (ethyl acetate:petroleum ether=2:1), to afford the target compound (239 mg, yield: 60.06%). LCMS (ESI) [M+H]+=411.00.

### Step 6: Preparation of (R)-4-(4-iodo-1-(1-((2-(trimethylsilyl)ethoxy)methyl)-1H-pyrazol-5-yl)-1H-pyrazole[3,4-b]pyridin-6-yl)-3-methylmorpholine

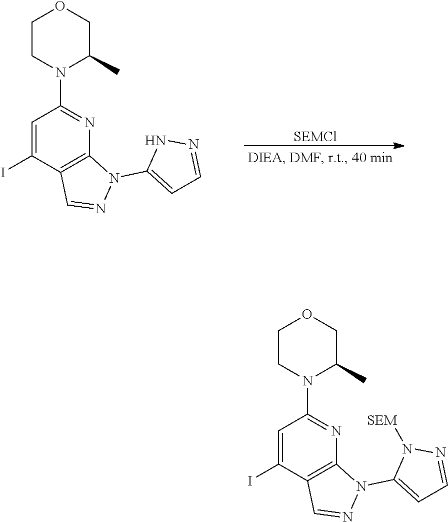 [SMILES: C[C@@H]1COCCN1c1cc(I)c2cnn(-c3ccn[nH]3)c2n1 ; "SEMCl" ; C[C@@H]1COCCN1c1cc(I)c2cnn(-c3ccnn3COCC[Si](C)(C)C)c2n1]

(R)-4-(4-Iodo-1-(1H-pyrazol-5-yl)-1H-pyrazole[3,4-b]pyridin-6-yl)-3-methylmorpholine (239 mg, 0.58 mmol, 1 equiv.) was dissolved in DMF (4 mL), 2-(trimethylsilyl)ethoxymethyl chloride (291.41 mg, 1.75 mmol, 3 equiv.) was added, followed by triethylamine (353.74 mg, 3.5 mmol, 6 equiv.), and the reaction liquid was stirred at room temperature for 1 hour. After the reaction was completed as monitored by LCMS, the reaction liquid was extracted with ethyl acetate (15 mL) and water (15 mL), the organic phase was concentrated, and the residue was purified by thin layer chromatography (ethyl acetate:petroleum ether=1:3), to afford the target compound (144 mg, yield: 45.73%). LCMS (ESI) [M+H]+=541.10.

### Step 7: Preparation of 3-methyl-4-(4,4,5,5-tetramethyl-1,3,2-dioxaborolan-2-yl)-5-(trifluoromethyl)-1H-pyrazole

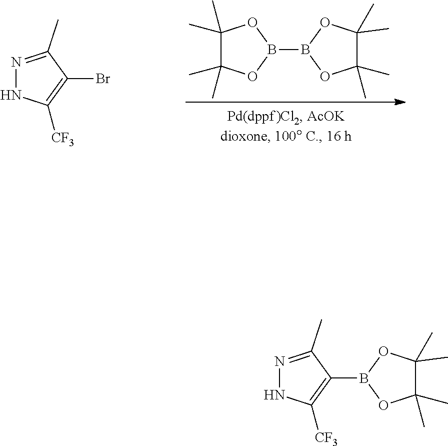

4-Bromo-3-methyl-5-(trifluoromethyl)-1H-pyrazole (100 mg, 0.44 mmol, 1 equiv.), bis(pinacolato)diboron (330 mg, 1.3 mmol, 3 equiv.), [1,1′-bis(diphenylphosphino)ferrocene]dichloropalladium (II) (10 mg, 0.04 mmol, 0.1 equiv.) and potassium acetate (214 mg, 2.2 mmol, 5 equiv.) were added to 1.4-dioxane solution (4 mL), and the mixture was reacted at 100° C. for 16 hours. After the reaction was completed as monitored by LCMS, the reaction liquid was filtered, the filtrate was extracted with water and ethyl acetate, the organic phase was concentrated, and the residue was purified by column chromatography (methanol:dichloromethane=0%-5%), to afford 3-methyl-4-(4,4,5,5-tetramethyl-1,3,2-dioxaborolan-2-yl)-5-(trifluoromethyl)-1H-pyrazole (20 mg, yield: 15.91%). LCMS (ESI) [M+H]+=277.05.

### Step 8: Preparation of (3R)-3-methyl-4-(4-(5-methyl-3-(trifluoromethyl)-1H-pyrazol-4-yl)-1-(1-((2-(trimethylsilyl)ethoxy)methyl)-1H-pyrazol-3-yl)-1H-pyrazolyl[3,4-b]pyridin-6-yl)morpholine

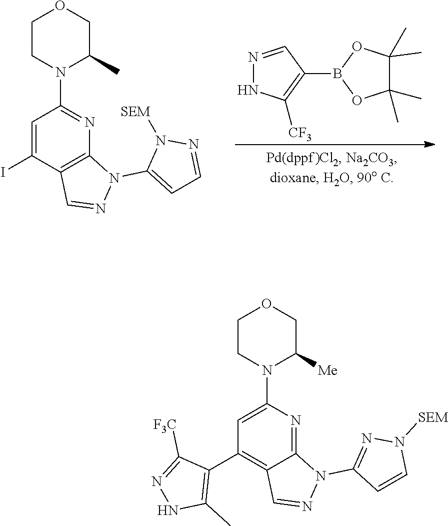 [SMILES: C[C@@H]1COCCN1c1cc(I)c2cnn(-c3ccnn3COCC[Si](C)(C)C)c2n1 ; CC1(C)OB(c2cn[nH]c2C(F)(F)F)OC1(C)C ; Cc1[nH]nc(C(F)(F)F)c1-c1cc(N2CCOC[C@H]2C)nc2c1cnn2-c1ccn(COCC[Si](C)(C)C)n1]

(R)-4-(4-Iodo-1-(1-((2-(trimethylsilyl)ethoxy)methyl)-1H-pyrazol-5-yl)-1H-pyrazole[3,4-b]pyridin-6-yl)-3-methylmorpholine (30 mg, 0.06 mmol, 1 equiv.) was added to a mixed solution of water (1 mL) and 1,4-dioxane (2 mL), then 3-methyl-4-(4,4,5,5-tetramethyl-1,3,2-dioxaborolan-2-yl)-5-(trifluoromethyl)-1H-pyrazole (18.6 mg, 0.07 mmol, 1.21 equiv.), sodium carbonate (12 mg, 0.11 mmol, 2.04 equiv.) and [1,1′-bis(diphenylphosphino)ferrocene]dichloropalladium (II) (4 mg) were added to the reaction liquid, and gas replacement was performed several times to ensure the absence of oxygen. The mixture was stirred and reacted at 90° C. for 16 hours. After a product was generated as monitored by LCMS, the reaction liquid was extracted three times with ethyl acetate and dried, the organic phases were combined and spun to dryness, and the residue was purified by thin layer chromatography (petroleum ether:ethyl acetate=2:1), to afford the target compound (4.2 mg, yield: 12.44%). LCMS (ESI) [M+H]+=563.20.

### Step 9: Preparation of (3R)-3-methyl-4-(4-(5-methyl-3-(trifluoromethyl)-1H-pyrazolyl-4-yl)-1-(1H-pyrazolyl-3-yl)-1H-pyrazolyl[3,4-b]pyridin-6-yl)morpholine

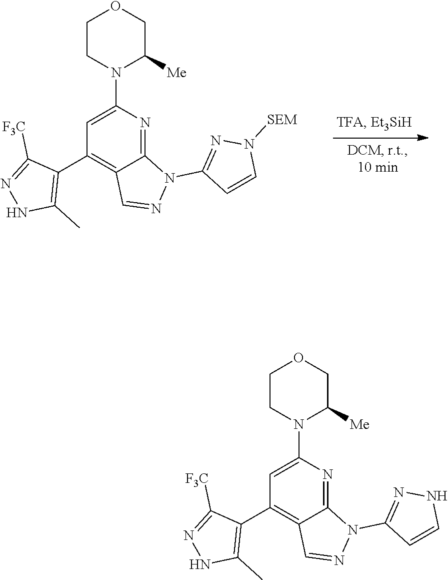 [SMILES: Cc1[nH]nc(C(F)(F)F)c1-c1cc(N2CCOC[C@H]2C)nc2c1cnn2-c1ccn(COCC[Si](C)(C)C)n1 ; Cc1[nH]nc(C(F)(F)F)c1-c1cc(N2CCOC[C@H]2C)nc2c1cnn2-c1cc[nH]n1]

(3R)-3-Methyl-4-(4-(5-methyl-3-(trifluoromethyl)-1H-pyrazol-4-yl)-1-(1-((2-(trimethylsilyl)ethoxy)methyl)-1H-pyrazol-3-yl)-1H-pyrazolyl[3,4-b]pyridin-6-yl)morpholine (4.2 mg, 0.01 mmol, 1 equiv.) was added to dichloromethane (0.3 mL), followed by triethylsilane (0.04 mL, 0.34 mmol, 46.08 equiv.) and trifluoroacetic acid (0.1 mL). The mixture was stirred at room temperature for 10 minutes. After the reaction was completed as monitored by LCMS, the reaction liquid was extracted three times with dichloromethane and dried over anhydrous sodium sulfate, the extract phases were combined and spun to dryness, and the residue was purified by thin layer chromatography (dichloromethane:methanol=15:1), to afford the target compound (1.2 mg, yield: 27.75%). LCMS (ESI) [M+H]+=433.15; ¹H NMR (399 MHz, DMSO-d6) δ 13.70 (s, 1H), 12.81 (s, 1H), 7.81 (d, J=15.6 Hz, 2H), 6.77 (s, 1H), 6.63 (s, 1H), 4.35 (d, J=3.6 Hz, 1H), 4.05 (d, J=11.9 Hz, 1H), 3.95 (dd, J=12.5, 4.0 Hz, 1H), 3.72 (d, J=11.4 Hz, 1H), 3.63 (dd, J=11.9, 2.5 Hz, 1H), 3.47 (td, J=11.8, 2.0 Hz, 1H), 3.14 (td, J=12.8, 3.8 Hz, 1H), 2.22 (s, 3H), 1.17 (d, J=6.7 Hz, 3H).

### Example 2

### Preparation of (R)-6-(6-(3-methylmorpholinyl)-1-(1H-pyrazol-3-yl)-1H-pyrazole[3,4-b]pyridin-4-yl)-1-oxaspiro[2.5]octan-6-ol

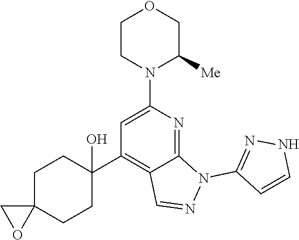

### Step 1: Preparation of A-methylenecyclohexanone

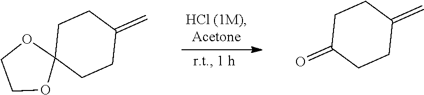

8-Methylene-1,4-dioxaspiro[4,5]decane (2 g, 12.97 mmol, 1 equiv.) was dissolved in acetone (30 mL), hydrochloric acid (30 mL, 1 M) was added, and the mixture was stirred at room temperature for 1 hour. After the reaction was completed as monitored by TLC (potassium permanganate color developer), the reaction liquid was extracted three times with ethyl acetate, and the organic phase was concentrated at low temperature (25° C.), to afford A-methylenecyclohexanone (2 g, crude).

### Step 2: Preparation of 1-oxaspiro[2.5]octan-6-one

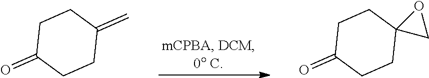

A-methylenecyclohexanone (1.3 g, 11.8 mmol, 1 equiv.) was added to dichloromethane (40 mL). m-Chloroperoxybenzoic acid (6.11 g, 35.41 mmol, 1.95 equiv.) was slowly added, and the mixture was stirred at 0° C. for 1 hour. Nuclear magnetic detection was performed, and the reaction liquid was spun to dryness at low temperature, to afford the crude 1-oxaspiro[2.5]octan-6-one (750 mg, 5.95 mmol, 50.38%). ¹H NMR (399 MHz, CDCl3) δ 2.81 (s, 2H), 2.71-2.59 (m, 2H), 2.42 (dt, J=9.7, 4.3 Hz, 2H), 2.19-2.10 (m, 2H), 1.81-1.71 (m, 2H).

### Step 3: Preparation of (R)-6-(6-(3-methylmorpholinyl)-1-(1-((2-(trimethylsilyl)ethoxy)methyl)-1H-pyrazol-5-yl)-1H-pyrazole[3,4-b]pyridin-4-yl)-1-oxaspiro[2.5]octan-6-ol

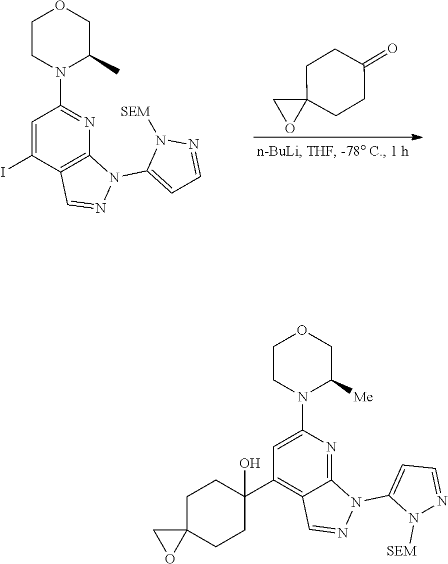 [SMILES: C[C@@H]1COCCN1c1cc(I)c2cnn(-c3ccnn3COCC[Si](C)(C)C)c2n1 ; O=C1CCC2(CC1)CO2 ; C[C@@H]1COCCN1c1cc(C2(O)CCC3(CC2)CO3)c2cnn(-c3ccnn3COCC[Si](C)(C)C)c2n1]

(R)-4-(4-Iodo-1-(1-((2-(trimethylsilyl)ethoxy)methyl)-1H-pyrazol-5-yl)-1H-pyrazole[3,4-b]pyridin-6-yl)-3-methylmorpholine (100 mg, 0.19 mmol, 1 equiv.) was added to tetrahydrofuran (3 mL), and 1-oxaspiro[2.5]octan-6-one (70 mg, 0.55 mmol, 3 equiv.) was added to the reaction liquid. n-Butyllithium (0.02 mL, 0.31 mmol, 1.69 equiv.) was slowly added at −78° C., and the mixture was stirred for 1 hour. After the raw materials were reacted completely as monitored by TLC, the reaction was quenched with saturated ammonium chloride aqueous solution, and the reaction liquid was extracted three times with dichloromethane. The extract phases were dried, combined and spun to dryness, and the residue was purified by thin layer chromatography (petroleum ether:ethyl acetate=2:1), to afford the target compound (22 mg, yield: 21.99%). LCMS (ESI) [M+H]⁺=541.10.

### Step 4: Preparation of (R)-6-(6-(3-methylmorpholinyl)-1-(1H-pyrazol-3-yl)-1H-pyrazole[3,4-b]pyridin-4-yl)-1-oxaspiro[2.5]octan-6-ol

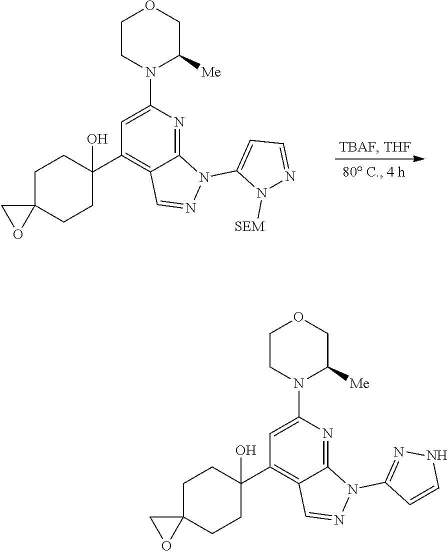 [SMILES: C[C@@H]1COCCN1c1cc(C2(O)CCC3(CC2)CO3)c2cnn(-c3ccnn3COCC[Si](C)(C)C)c2n1 ; C[C@@H]1COCCN1c1cc(C2(O)CCC3(CC2)CO3)c2cnn(-c3cc[nH]n3)c2n1]

(R)-6-(6-(3-Methylmorpholinyl)-1-(1-((2-(trimethylsilyl)ethoxy)methyl)-1H-pyrazol-5-yl)-1H-pyrazole[3,4-b]pyridin-4-yl)-1-oxaspiro[2.5]octan-6-ol (22 mg, 0.04 mmol, 1 equiv.) was added to tetrahydrofuran (2 mL), followed by tetrabutylammonium fluoride (0.2 mL, 0.76 mmol, 18.78 equiv.), and the mixture was heated to 80° C. and stirred for 4 hours. After the raw materials were reacted completely as monitored by TLC, the reaction liquid was extracted three times with ethyl acetate, and the organic phases were dried and combined. The residue was purified by thin layer chromatography (dichloromethane:methanol=10:1), and a product was scraped off, to afford the target product (9.0 mg, yield: 53.89%). LCMS (ESI) [M+H]+=411.40; ¹H NMR (400 MHz, CD3OD) δ 8.21 (s, 1H), 7.72 (s, 1H), 6.93 (s, 1H), 6.85 (s, 1H), 4.54 (d, J=8.4 Hz, 1H), 4.12-4.00 (m, 2H), 3.84-3.75 (m, 2H), 3.60 (d, J=11.5 Hz, 1H), 3.37-3.31 (m, 1H), 2.76 (s, 2H), 2.46 (td, J=12.6, 3.2 Hz, 2H), 2.30 (t, J=12.8 Hz, 2H), 2.03 (d, J=13.4 Hz, 2H), 1.29 (d, J=7.1 Hz, 5H).

### Example 3

### Preparation of (3R)-3-methyl-4-(4-(3-methyl-1-(piperidin-4-yl)-5-(trifluoromethyl)-1H-pyrazol-4-yl)-1-(1H-pyrazol-3-yl)-1H-pyrazole[3,4-b]pyridin-6-yl)morpholine

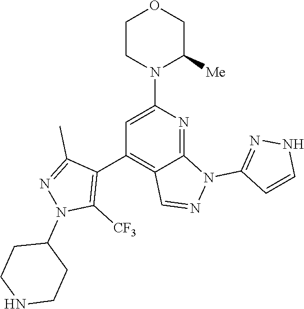

### Step 1: Preparation of tert-butyl 4-(methylsulfonyl)oxy)piperidine-1-carboxylate

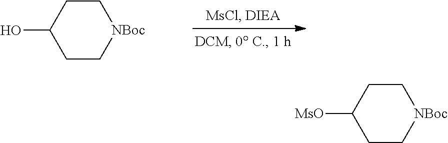 [SMILES: CC(C)(C)OC(=O)N1CCC(O)CC1 ; CC(C)(C)OC(=O)N1CCC(OS(C)(=O)=O)CC1]

Tert-butyl 4-hydroxypiperidine-1-carboxylate (50 mg, 0.25 mmol, 1 equiv.) and N,N-diisopropylethylamine (96.32 mg, 0.75 mmol, 3 equiv.) were added to dichloromethane (5 mL), and the mixture was cooled to 0° C. and stirred for 10 minutes after nitrogen replacement was performed three times. Methanesulfonyl chloride (42.69 mg, 0.37 mmol, 1.5 equiv.) was slowly added to the reaction liquid, and the resulting mixture was stirred for additional 1 hour. After the reaction was completed as monitored by TLC, the reaction liquid was diluted with water and extracted three times with dichloromethane, and the organic phases were combined, washed with saturated sodium chloride aqueous solution, dried, filtered and concentrated, to afford the crude target compound (40 mg, yield: 57.64%).

### Step 2: Preparation of tert-butyl 4-(5-methyl-4-(6-((R)-3-methylmorpholine)-1-(1-((2-(trimethylsilyl)ethoxy)methyl)-1H-pyrazol-3-yl)-1H-pyrazole[3,4-b]pyridin-4-yl)-3-(trifluoromethyl)-1H-pyrazol-1-yl)piperidine-1-carboxylate

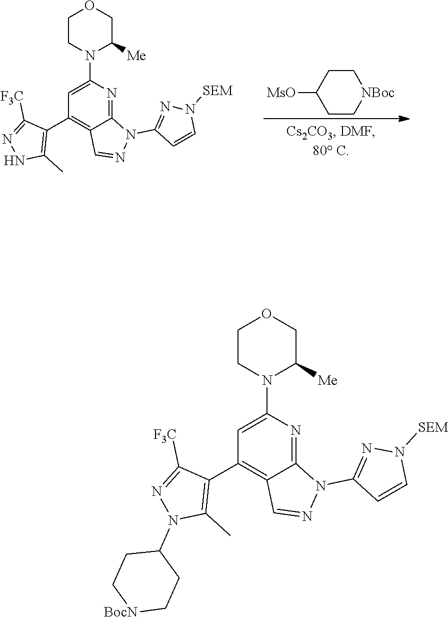 [SMILES: Cc1[nH]nc(C(F)(F)F)c1-c1cc(N2CCOC[C@H]2C)nc2c1cnn2-c1ccn(COCC[Si](C)(C)C)n1 ; CC(C)(C)OC(=O)N1CCC(OS(C)(=O)=O)CC1 ; Cc1c(-c2cc(N3CCOC[C@H]3C)nc3c2cnn3-c2ccn(COCC[Si](C)(C)C)n2)c(C(F)(F)F)nn1C1CCN(C(=O)OC(C)(C)C)CC1]

Tert-butyl 4-(methylsulfonyl)oxy)piperidine-1-carboxylate (30 mg, 0.05 mmol, 1 equiv.), (3R)-3-methyl-4-(4-(5-methyl-3-(trifluoromethyl)-1H-pyrazol-4-yl)-1-(1-((2-(trimethylsilyl)ethoxy)methyl)-1H-pyrazol-3-yl)-1H-pyrazole[3,4-b]pyridin-6-yl)morpholine (22.34 mg, 0.08 mmol, 1.5 equiv.) and cesium carbonate (52.12 mg, 0.16 mmol, 3 equiv.) were added to N,N-dimethylformamide (5 mL) at room temperature, and the mixture was heated to 80° C. and stirred for 16 hours after nitrogen replacement was performed three times. After the reaction was completed as monitored by LCMS, the reaction liquid was diluted with water and extracted three times with ethyl acetate, the organic phases were combined, washed with saturated sodium chloride aqueous solution, dried, filtered and concentrated, and the concentrate was purified by thin layer chromatography (TLC) (petroleum ether:ethyl acetate=1:1), to afford the target compound (35 mg, yield: 88%). LCMS (ESI) [M+H]+=746.91.

### Step 3: Preparation of (3R)-3-methyl-4-(4-(3-methyl-1-(piperidin-4-yl)-5-(trifluoromethyl)-1H-pyrazol-4-yl)-1-(1H-pyrazol-3-yl)-1H-pyrazole[3,4-b]pyridin-6-yl)morpholine

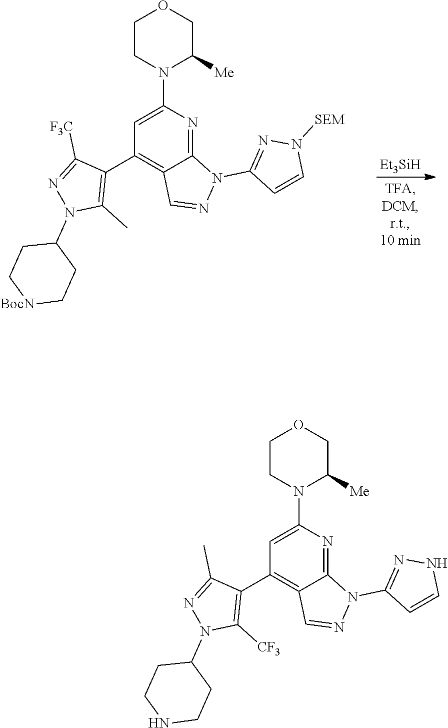 [SMILES: "Et3SiH" ; Cc1c(-c2cc(N3CCOC[C@H]3C)nc3c2cnn3-c2ccn(COCC[Si](C)(C)C)n2)c(C(F)(F)F)nn1C1CCN(C(=O)OC(C)(C)C)CC1 ; Cc1nn(C2CCNCC2)c(C(F)(F)F)c1-c1cc(N2CCOC[C@H]2C)nc2c1cnn2-c1cc[nH]n1]

Tert-butyl 4-(5-methyl-4-(6-((R)-3-methylmorpholine)-1-(1-((2-(trimethylsilyl)ethoxy)methyl)-1H-pyrazol-3-yl)-1H-pyrazole[3,4-b]pyridin-4-yl)-3-(trifluoromethyl)-1H-pyrazol-1-yl)piperidine-1-carboxylate (100 mg, 0.13 mmol, 1 equiv.) and triethylsilane (0.57 ml, 4.91 mmol, 36.66 equiv.) were dissolved in a mixed solution of dichloromethane (2 mL) and trifluoroacetic acid (2 mL), and the mixture was stirred at room temperature for 10 minutes after nitrogen replacement was performed three times. After the reaction was completed as monitored by TLC, the reaction liquid was adjusted to pH 9 with saturated sodium bicarbonate aqueous solution and then extracted three times with dichloromethane, the organic phases were combined, washed with saturated sodium chloride aqueous solution, dried, filtered and concentrated, and the concentrate was purified by preparative chromatography, to afford the target product (20.8 mg, yield: 31.04%). LCMS (ESI) [M+H]+=516.53; ¹H NMR (400 MHz, DMSO-d6) 8.30 (s, 1H), 7.80 (s, 1H), 6.78 (d, J=2.0 Hz, 1H), 6.65 (s, 1H), 4.41 (d, J=50.5 Hz, 2H), 4.06 (d, J=11.6 Hz, 1H), 3.95 (d, J=7.4 Hz, 1H), 3.72 (d, J=12.1 Hz, 2H), 3.62 (d, J=11.2 Hz, 2H), 3.15 (d, J=11.4 Hz, 4H), 2.74 (s, 2H), 2.26 (s, 3H), 1.96 (s, 4H), 1.17 (d, J=6.6 Hz, 3H).

### Example 4

### Preparation of (3R)-3-methyl-4-(4-(5-methyl-1-(piperidin-4-yl)-3-(trifluoromethyl)-1H-pyrazol-4-yl)-1-(1H-pyrazol-3-yl)-1H-pyrazole[3,4-b]pyridin-6-yl)morpholine

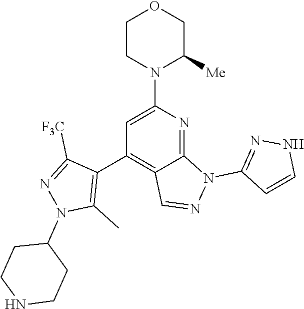

### Step 1: Preparation of (3R)-3-methyl-4-(4-(5-methyl-1-(piperidin-4-yl)-3-(trifluoromethyl)-1H-pyrazol-4-yl)-1-(1H-pyrazol-3-yl)-1H-pyrazole[3,4-b]pyridin-6-yl)morpholine

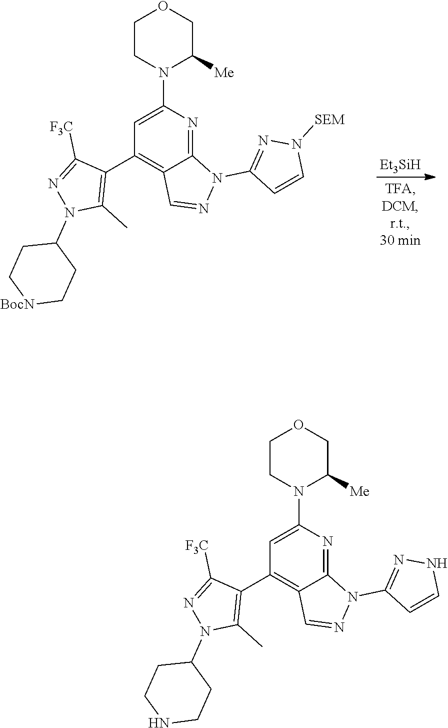 [SMILES: "Et3SiH" ; Cc1c(-c2cc(N3CCOC[C@H]3C)nc3c2cnn3-c2ccn(COCC[Si](C)(C)C)n2)c(C(F)(F)F)nn1C1CCN(C(=O)OC(C)(C)C)CC1 ; Cc1c(-c2cc(N3CCOC[C@H]3C)nc3c2cnn3-c2cc[nH]n2)c(C(F)(F)F)nn1C1CCNCC1]

Tert-butyl 4-(5-methyl-4-(6-((R)-3-methylmorpholine)-1-(1-((2-(trimethylsilyl)ethoxy)methyl)-1H-pyrazol-3-yl)-1H-pyrazole[3,4-b]pyridin-4-yl)-3-(trifluoromethyl)-1H-pyrazol-1-yl)piperidine-1-carboxylate (30 mg, 0.04 mmol, 1 equiv.) was dissolved in trifluoroacetic acid (2 mL) and dichloromethane (2 mL), triethylsilane (0.2 mL) was added, and the mixture was stirred at room temperature for 30 minutes. After the reaction was completed as monitored by LCMS, the reaction liquid was adjusted to be alkaline with saturated sodium bicarbonate aqueous solution and extracted with ethyl acetate and water, the organic phases were combined, dried, filtered and concentrated, and the residue was purified by thin layer chromatography (dichloromethane:methanol=8:1), to afford the target compound (4.9 mg, yield: 23.63%). LCMS (ESI) [M+H]+=516.20; ¹H NMR (399 MHz, CD3OD) δ 7.75 (s, 1H), 7.70 (s, 1H), 7.46-7.17 (m, 1H), 6.96 (s, 1H), 6.66 (s, 1H), 4.45 (d, J=13.0 Hz, 2H), 4.08 (dd, J=47.7, 11.2 Hz, 2H), 3.84-3.75 (m, 2H), 3.63 (t, J=11.5 Hz, 1H), 3.34 (s, 1H), 3.23 (s, 2H), 2.78 (t, J=13.0 Hz, 2H), 2.23 (d, J=20.5 Hz, 2H), 2.14 (s, 3H), 2.08-1.97 (m, 2H), 1.30 (d, J=6.3 Hz, 3H).

### Example 5

### Preparation of (R)-4-(4-(3-methoxypropyl-1-alkynyl)-1-(1H-pyrazol-3-yl)-1H-pyrazole[3,4-b]pyridin-6-yl)-3-methylmorpholine

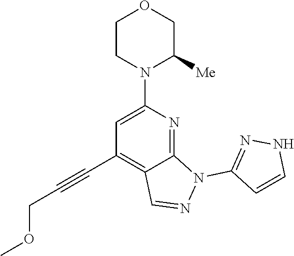

### Step 1: Preparation of (R)-4-(4-(3-methoxypropyl-1-alkynyl)-1-(1-((2-(trimethylsilyl)ethoxy)methyl)-1H-pyrazol-3-yl)-1H-pyrazole[3,4-b]pyridin-6-yl)-3-methylmorpholine

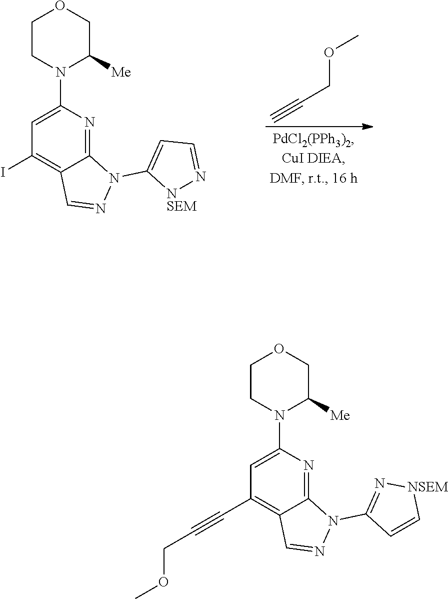

(R)-4-(4-Iodo-1-(1-((2-(trimethylsilyl)ethoxy)methyl)-1H-pyrazol-5-yl)-1H-pyrazole[3,4-b]pyridin-6-yl)-3-methylmorpholine (50 mg, 0.09 mmol, 1 equiv.), 3-methoxypropyne (13.62 mg, 0.19 mmol, 2.1 equiv.), bis(triphenylphosphine)dichloropalladium (II) (1.95 mg, 0 mmol, 0.03 equiv.), cuprous iodide (0.88 mg, 0 mmol, 0.05 equiv.) and diisopropylethylamine (38.26 mg, 0.3 mmol, 3.2 equiv.) were dissolved in N,N-dimethylformamide (4 mL), and the mixture was stirred overnight at room temperature. After the reaction was completed as monitored by LCMS, the reaction liquid was extracted with ethyl acetate and water, the organic phases were combined, dried, filtered and spun to dryness, and the residue was separated and purified by a TLC plate (petroleum ether:ethyl acetate=2:1), to afford the target compound (34 mg, yield: 76.15%). LCMS (ESI) [M+H]+=483.45

### Step 2: Preparation of (R)-4-(4-(3-methoxypropyl-1-alkynyl)-1-(1H-pyrazol-3-yl)-1H-pyrazole[3,4-b]pyridin-6-yl)-3-methylmorpholine

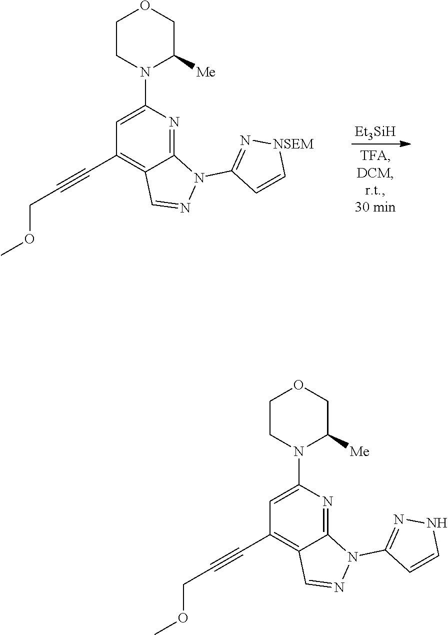 [SMILES: "Et3SiH" ; COCC#Cc1cc(N2CCOC[C@H]2C)nc2c1cnn2-c1ccn(COCC[Si](C)(C)C)n1 ; COCC#Cc1cc(N2CCOC[C@H]2C)nc2c1cnn2-c1cc[nH]n1]

(R)-4-(4-(3-Methoxypropyl-1-alkynyl)-1-(1-((2-(trimethylsilyl)ethoxy)methyl)-1H-pyrazol-3-yl)-1H-pyrazole[3,4-b]pyridin-6-yl)-3-methylmorpholine (30 mg, 0.04 mmol, 1 equiv.) was dissolved in trifluoroacetic acid (2 mL) and dichloromethane (2 mL), triethylsilane (0.2 mL) was added, and the mixture was stirred at room temperature for 30 minutes. After the reaction was completed as monitored by LCMS, the reaction liquid was adjusted to be alkaline with saturated sodium bicarbonate aqueous solution and extracted with ethyl acetate and water, the organic phases were combined, dried, filtered and concentrated, and the residue was purified by thin layer chromatography (dichloromethane:methanol=10:1), to afford the target compound (14.3 mg, yield: 57.61%). LCMS (ESI) [M+H]+=353.35; ¹H NMR (399 MHz, CD3OD) δ 7.98 (s, 1H), 7.73 (s, 1H), 6.90 (s, 1H), 6.83 (s, 1H), 4.45 (s, 1H), 4.42 (s, 2H), 4.03 (dd, J=27.5, 10.3 Hz, 2H), 3.81-3.72 (m, 2H), 3.59 (t, J=10.4 Hz, 1H), 3.47 (s, 3H), 3.25 (s, 1H), 1.27 (d, J=6.7 Hz, 3H).

### Example 6

### Preparation of (R)-1-((6-(3-methylmorpholinyl)-1-(1H-pyrazolyl-3-yl)-1H-pyrazolyl[3,4-b]pyridin-4-yl)ethynyl)cyclohexanol

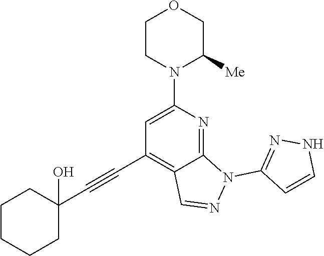

### Step 1: Preparation of (R)-1-((6-(3-methylmorpholinyl)-1-(1-((2-(trimethylsilyl)ethoxy)methyl)-1H-pyrazolyl-3-yl)-1H-pyrazolyl[3,4-b]pyridin-4-yl)ethynyl)cyclohexanol

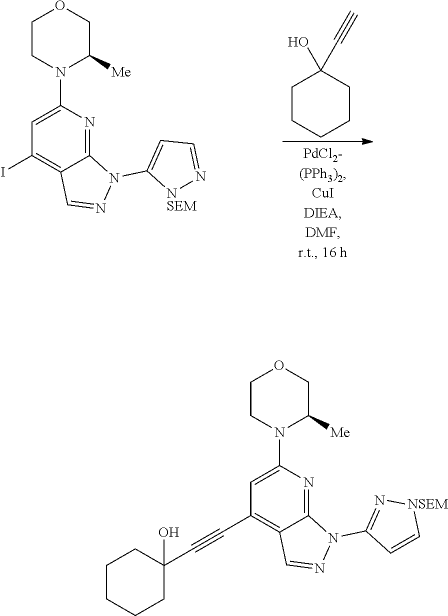

(R)-4-(4-Iodo-1-(1-((2-(trimethylsilyl)ethoxy)methyl)-1H-pyrazol-5-yl)-1H-pyrazole[3,4-b]pyridin-6-yl)-3-methylmorpholine (50 mg, 0.09 mmol, 1 equiv.), 1-ethynylcyclohexanol (23.5 mg, 0.19 mmol, 2.1 equiv.), bis(triphenylphosphine)dichloropalladium (II) (1.89 mg, 0.0027 mmol, 0.03 equiv.), cuprous iodide (0.86 mg, 0.0045 mmol, 0.05 equiv.) and diisopropylethylamine (37.2 mg, 0.29 mmol, 3.2 equiv.) were dissolved in N,N-dimethylformamide (4 mL), and the mixture was stirred overnight at room temperature. After the reaction was completed as monitored by LCMS, the reaction liquid was extracted with ethyl acetate and water, the organic phases were combined, dried, filtered and spun to dryness, and the residue was purified by prep TLC (petroleum ether:ethyl acetate=1:1), to afford the target compound (40 mg, yield: 82.65%). LCMS (ESI) [M+H]+=537.25.

### Step 2: Preparation of (R)-1-((6-(3-methylmorpholinyl)-1-(1H-pyrazolyl-3-yl)-1H-pyrazolyl[3,4-b]pyridin-4-yl)ethynyl)cyclohexanol

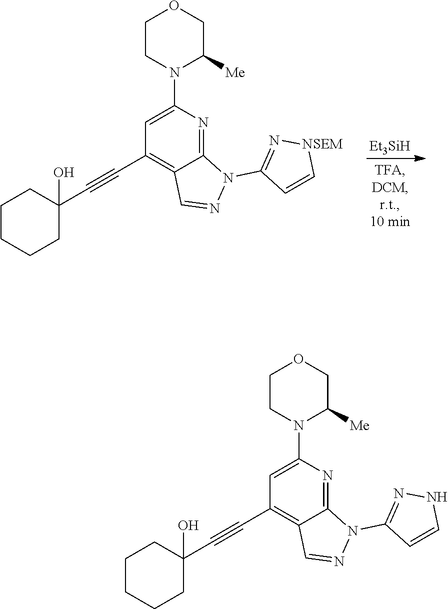 [SMILES: "Et3SiH" ; C[C@@H]1COCCN1c1cc(C#CC2(O)CCCCC2)c2cnn(-c3ccn(COCC[Si](C)(C)C)n3)c2n1 ; C[C@@H]1COCCN1c1cc(C#CC2(O)CCCCC2)c2cnn(-c3cc[nH]n3)c2n1]

(R)-1-((6-(3-Methylmorpholinyl)-1-(1-((2-(trimethylsilyl)ethoxy)methyl)-1H-pyrazolyl-3-yl)-1H-pyrazolyl[3,4-b]pyridin-4-yl)ethynyl)cyclohexanol (40 mg, 0.07 mmol, 1 equiv.) was dissolved in trifluoroacetic acid (1 mL) and dichloromethane (1 mL), triethylsilane (0.1 mL) was added, and the mixture was stirred at room temperature for 10 minutes. After the reaction was completed as monitored by LCMS, the reaction liquid was adjusted to be alkaline with saturated sodium bicarbonate aqueous solution and extracted with ethyl acetate and water, the organic phases were combined, dried, filtered and concentrated, and the residue was purified by thin layer chromatography (dichloromethane:methanol=10:1), to afford the target compound (10.5 mg, yield: 36.9%). LCMS (ESI) [M+H]+=407.20; ¹H NMR (399 MHz, CD3OD) δ 7.98 (s, 1H), 7.74 (s, 1H), 6.91 (s, 1H), 6.80 (s, 1H), 4.62-4.44 (m, 2H), 4.04 (dd, J=29.4, 12.2 Hz, 2H), 3.79 (t, J=12.5 Hz, 2H), 3.63-3.56 (m, 1H), 2.04 (d, J=11.7 Hz, 2H), 1.81-1.75 (m, 2H), 1.67 (t, J=10.3 Hz, 6H), 1.28 (d, J=6.7 Hz, 3H).

### Example 7

### Preparation of (R)-1-(4-(6-(3-methylmorpholinyl)-1-(1H-pyrazolyl-3-yl)-1H-pyrazolyl[3,4-b]pyridin-4-yl)phenyl)piperidin-2-one

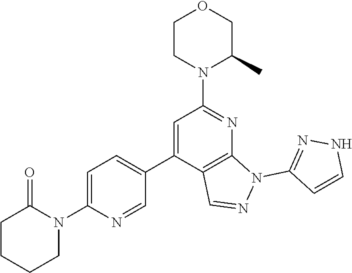

### Step 1: Preparation of 5-bromo-N-(4-(4,4,5,5-tetramethyl-1,3,2-dioxaborolan-2-yl)phenyl)pentaamide

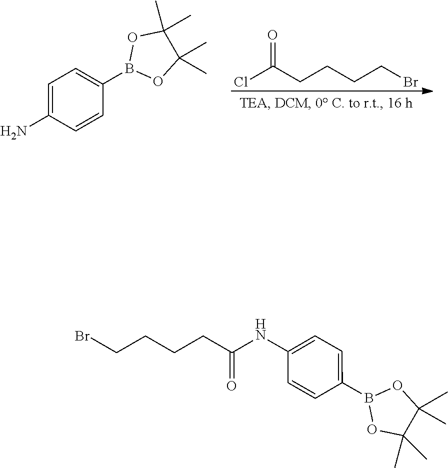

p-Aminophenylboronic acid (500 mg, 2.28 mmol, 1.0 equiv.) and triethylamine (346 mg, 3.42 mmol, 1.5 equiv) were weighed and dissolved in dichloromethane (10 mL), then 5-bromopentanoyl chloride (500 mg, 2.51 mmol, 1.1 equiv.) was added under ice bath, and the mixture was heated to room temperature and reacted for 16 hours after nitrogen replacement was performed three times. After the raw materials were reacted completely as monitored by TLC and a product was generated as monitored by LCMS, the reaction liquid was extracted with water (100 mL) and ethyl acetate (50 mL×3), the organic phases were combined and spun to dryness, and the residue was purified by column chromatography (petroleum ether:ethyl acetate=20/1-5/1), to afford the target compound (868 mg, yield: 99.8%). LCMS (ESI) [M+H]⁺=382.05.

### Step 2: Preparation of 1-(4-(4,4,5,5-tetramethyl-1,3,2-dioxaborolan-2-yl)phenyl)piperidin-2-one

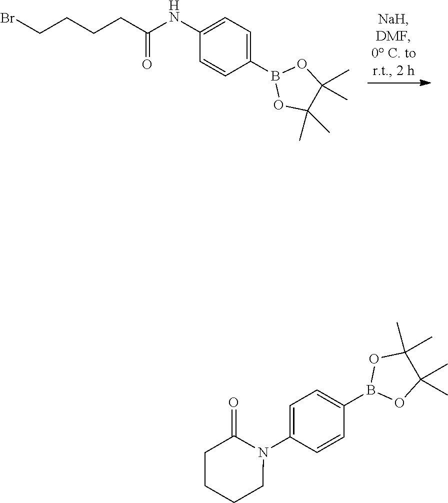 [SMILES: CC1(C)OB(c2ccc(NC(=O)CCCCBr)cc2)OC1(C)C ; CC1(C)OB(c2ccc(N3CCCCC3=O)cc2)OC1(C)C]

5-Bromo-N-(4-(4,4,5,5-tetramethyl-1,3,2-dioxaborolan-2-yl)phenyl)pentaamide (868 mg, 2.28 mmol, 1.0 equiv.) was dissolved in N,N-dimethylformamide (10 mL), then sodium hydride (136 mg, 2.42 mmol, 1.5 equiv) was added under ice bath, and then the mixture was reacted at room temperature for 2 hours after nitrogen replacement was performed three times. After the raw materials were reacted completely as monitored by TLC and a product was generated as monitored by LCMS, the reaction was quenched with water (50 mL), and then the reaction liquid was extracted with ethyl acetate (50 mL×3). The organic phases were combined and spun to dryness, and the residue was purified by thin layer chromatography (petroleum ether:ethyl acetate=3:1), to afford the product (300 mg, yield: 43.6%). LCMS (ESI) [M+H]⁺=302.1.

### Step 3: Preparation of (R)-1-(4-(6-(3-methylmorpholinyl)-1-(1-(2-(trimethylsilyl)ethoxy)methyl)-1H-pyrazol-3-yl)-1H-pyrazole[3,4-b]pyridin-4-yl)phenyl)piperidin-2-one

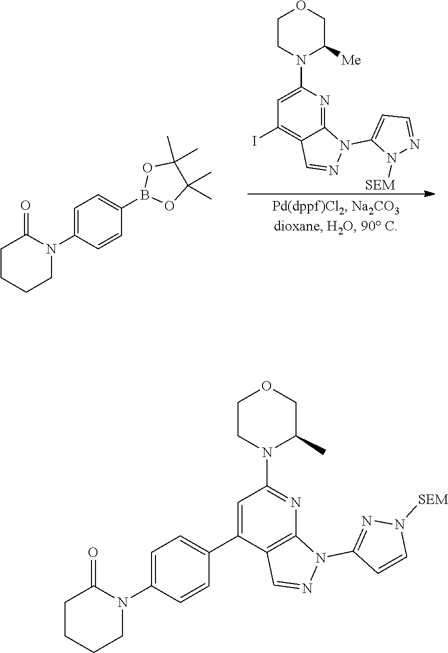 [SMILES: CC1(C)OB(c2ccc(N3CCCCC3=O)cc2)OC1(C)C ; C[C@@H]1COCCN1c1cc(I)c2cnn(-c3ccnn3COCC[Si](C)(C)C)c2n1 ; C[C@@H]1COCCN1c1cc(-c2ccc(N3CCCCC3=O)cc2)c2cnn(-c3ccn(COCC[Si](C)(C)C)n3)c2n1]

1-(4-(4,4,5,5-Tetramethyl-1,3,2-dioxaborolan-2-yl)phenyl)piperidin-2-one (60 mg, 0.198 mmol, 1.1 equiv.), (R)-4-(4-iodo-1-(1-((2-(trimethylsilyl)ethoxy)methyl)-1H-pyrazol-5-yl)-1H-pyrazole[3,4-b]pyridin-6-yl)-3-methylmorpholine (100 mg, 0.185 mmol, 1.0 equiv.), [1,1′-bis(diphenylphosphino)ferrocene]dichloropalladium (II) (14 mg, 0.018 mmol, 0.1 equiv) and sodium carbonate (40 mg, 0.37 mmol, 2.0 equiv.) were dissolved in 1,4-dioxane (4 mL) and water (2 mL), and then the mixture was heated to 90° C. and reacted at this temperature for 3 hours after nitrogen replacement was performed three times. After the raw materials were reacted completely as monitored by TLC and a product was generated as monitored by LCMS, the reaction liquid was filtered and then extracted with ethyl acetate (20 mL×3), the organic phases were combined and spun to dryness, and the residue was purified by thin layer chromatography (petroleum ether:ethyl acetate=1:1), to afford the target compound (88 mg, yield: 75.7%). LCMS (ESI) [M+H]+=588.25;

### Step 4: Preparation of (R)-1-(4-(6-(3-methylmorpholinyl)-1-(1H-pyrazolyl-3-yl)-1H-pyrazolyl[3,4-b]pyridin-4-yl)phenyl)piperidin-2-one

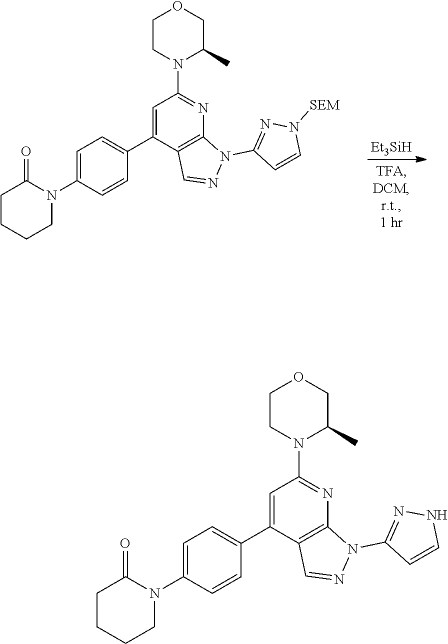 [SMILES: "Et3SiH" ; C[C@@H]1COCCN1c1cc(-c2ccc(N3CCCCC3=O)cc2)c2cnn(-c3ccn(COCC[Si](C)(C)C)n3)c2n1 ; C[C@@H]1COCCN1c1cc(-c2ccc(N3CCCCC3=O)cc2)c2cnn(-c3cc[nH]n3)c2n1]

(R)-1-(4-(6-(3-Methylmorpholinyl)-1-(1-(2-(trimethylsilyl)ethoxy)methyl)-1H-pyrazol-3-yl)-1H-pyrazole[3,4-b]pyridin-4-yl)phenyl)piperidin-2-one (88 mg, 0.15 mmol, 1.0 equiv.) was dissolved in dichloromethane (4 mL), then trifluoroacetic acid (4 mL) and triethylsilane (0.4 mL) were added, and then the mixture was reacted at 25° C. for 1 hour after nitrogen replacement was performed three times. After the raw materials were reacted completely as monitored by TLC and a product was generated as monitored by LCMS, the reaction was quenched with saturated sodium bicarbonate (50 mL), and then the reaction liquid was extracted with ethyl acetate (20 mL×3). The organic phases were combined and spun to dryness, and the residue was purified by thin layer chromatography (dichloromethane/methanol=10:1), to afford the target compound (37 mg, yield: 53.9%). LCMS (ESI) [M+H]+=458.20; ¹H NMR (400 MHz, DMSO-d6) δ 12.82 (s, 1H), 8.14 (s, 1H), 7.87-7.80 (m, 3H), 7.45 (d, J=8.3 Hz, 2H), 6.90 (s, 1H), 6.80 (s, 1H), 4.53 (s, 1H), 4.13 (d, J=12.5 Hz, 1H), 3.95 (d, J=9.4 Hz, 1H), 3.74 (d, J=11.1 Hz, 1H), 3.67-3.62 (m, 3H), 3.49 (t, J=10.7 Hz, 1H), 3.18 (t, J=11.6 Hz, 1H), 2.41 (t, J=5.9 Hz, 2H), 1.85 (s, 4H), 1.22-1.17 (m, 3H).

### Example 8

### Preparation of (R)-4-(4,6-dihydro-2H-pyran-3-yl)-1-(1H-pyrazol-3-yl)-1H-pyrazole[3,4-b]pyridin-6-yl)-3-methylmorpholine

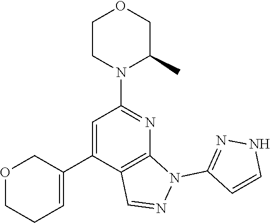

### Step 1: Preparation of 3,4-dihydro-2H-pyran-5-yl trifluoromethanesulfonate

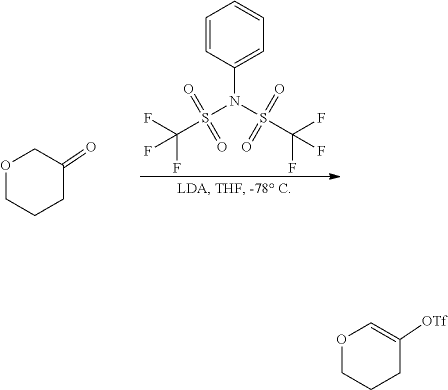 [SMILES: O=C1CCCOC1 ; O=S(=O)(N(c1ccccc1)S(=O)(=O)C(F)(F)F)C(F)(F)F ; O=S(=O)(OC1=COCCC1)C(F)(F)F]

Tetrahydro-2H-pyran-3-one (500 mg, 4.99 mmol, 1 equiv.) was dissolved in tetrahydrofuran (10 mL), lithium diisopropylamide (3 mL, 6.0 mmol, 1.2 equiv.) was added at −78° C., and the mixture was stirred at −78° C. for 45 minutes. 1, 1, 1-Trifluoro-N-phenyl-N-((trifluoromethyl)sulfonyl)methanesulfonamide (1.96 g, 5.49 mmol, 1.1 equiv.) was added, and the resulting mixture was stirred at room temperature for 3 hours. After the reaction was completed as monitored by TLC (potassium permanganate color developer), the reaction was quenched with saturated ammonium chloride aqueous solution, and the reaction liquid was extracted with ethyl acetate and water. The organic phases were combined, dried, filtered and spun to dryness, and the residue was purified by column chromatography (petroleum ether:ethyl acetate=100:1), to afford the target compound (200 mg, yield: 17.25%).

### Step 2: Preparation of 2-(3,4-dihydro-2H-pyran-5-yl)-4,4,5,5-tetramethyl-1,3,2-dioxaborolane

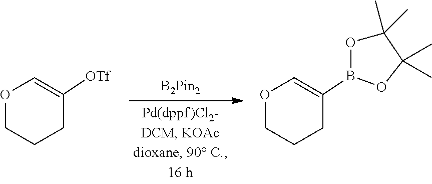

3,4-Dihydro-2H-pyran-5-yl trifluoromethanesulfonate (200 mg, 0.86 mmol, 1 equiv.), bis(pinacolato)diboron (328.12 mg, 1.29 mmol, 1.5 equiv.), [1,1′-bis(diphenylphosphino)ferrocene]dichloropalladium (II) (63.2 mg, 0.09 mmol, 0.1 equiv.) and potassium acetate (253.61 mg, 2.58 mmol, 3 equiv.) were dissolved in 1, 4-dioxane (5 mL), and the mixture was stirred overnight at 90° C. after nitrogen replacement was performed three times. After the reaction was completed as monitored by TLC (potassium permanganate color developer), the reaction liquid was filtered and extracted with ethyl acetate and water, the organic phases were combined, dried, filtered and concentrated, and the residue was purified by prepTLC (petroleum ether:ethyl acetate=5:1), to afford the target compound (50 mg, yield: 27.63%).

### Step 3: Preparation of (R)-4-(4-(5,6-dihydro-2H-pyran-3-yl)-1-(1-(2-(trimethylsilyl)ethoxy)methyl)-1H-pyrazol-3-yl)-1H-pyrazole[3,4-b]pyridin-6-yl)-3-methylmorpholine

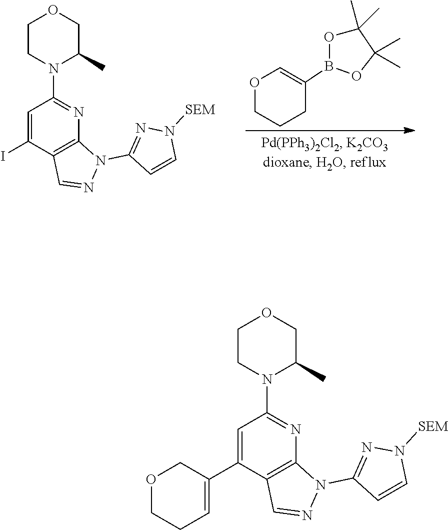 [SMILES: C[C@@H]1COCCN1c1cc(I)c2cnn(-c3ccn(COCC[Si](C)(C)C)n3)c2n1 ; CC1(C)OB(C2=COCCC2)OC1(C)C ; C[C@@H]1COCCN1c1cc(C2=CCCOC2)c2cnn(-c3ccn(COCC[Si](C)(C)C)n3)c2n1]

2-(3,4-Dihydro-2H-pyran-5-yl)-4,4,5,5-tetramethyl-1,3,2-dioxaborolane (60 mg, 0.11 mmol, 1 equiv.), (R)-4-(4-iodo-1-(1-((2-(trimethylsilyl)ethoxy)methyl)-1H-pyrazol-3-yl)-1H-pyrazole[3,4-b]pyridin-6-yl)-3-methylmorpholine (27.99 mg, 0.13 mmol, 1.2 equiv.), sodium carbonate (23.53 mg, 0.22 mmol, 2 equiv.) and [1,1′-bis(diphenylphosphino)ferrocene]dichloropalladium (II) (8.15 mg, 0.01 mmol, 0.1 equiv.) were dissolved in 1,4-dioxane (4 mL) and water (2 mL), and the mixture was stirred at 90° C. for 2 hours. After the reaction was completed as monitored by LCMS, the reaction liquid was extracted with ethyl acetate and water, the organic phases were combined, dried, filtered and spun to dryness, and the residue was purified by prepTLC (petroleum ether:ethyl acetate=1:1), to afford the target compound (20 mg, yield: 36.27%). LCMS (ESI) [M+H]⁺=497.30.

### Step 4: Preparation of (R)-4-(4,6-dihydro-2H-pyran-3-yl)-1-(1H-pyrazol-3-yl)-1H-pyrazole[3,4-b]pyridin-6-yl)-3-methylmorpholine

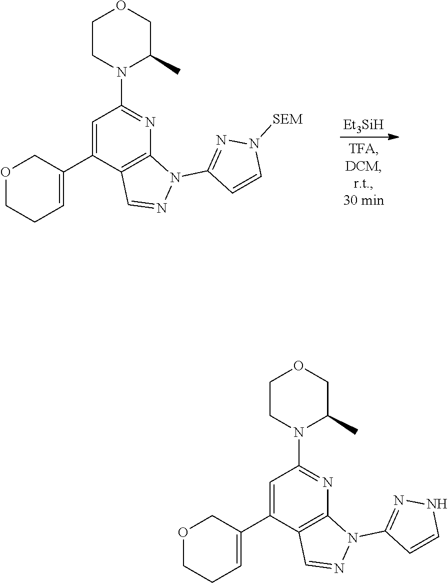 [SMILES: "Et3SiH" ; C[C@@H]1COCCN1c1cc(C2=CCCOC2)c2cnn(-c3ccn(COCC[Si](C)(C)C)n3)c2n1 ; C[C@@H]1COCCN1c1cc(C2=CCCOC2)c2cnn(-c3cc[nH]n3)c2n1]

(R)-4-(4-(5,6-Dihydro-2H-pyran-3-yl)-1-(1-(2-(trimethylsilyl)ethoxy)methyl)-1H-pyrazol-3-yl)-1H-pyrazole[3,4-b]pyridin-6-yl)-3-methylmorpholine (20 mg, 0.04 mmol, 1 equiv.) was dissolved in trifluoroacetic acid (2 mL) and dichloromethane (2 mL), triethylsilane (0.2 mL) was added, and the mixture was stirred at room temperature for 30 minutes. After the reaction was completed as monitored by LCMS, the reaction liquid was adjusted to be alkaline with saturated sodium bicarbonate aqueous solution and extracted with ethyl acetate and water, the organic phases were combined, dried, filtered and concentrated, and the residue was purified by prepTLC (dichloromethane:methanol=10:1), to afford the target compound (9.5 mg, yield: 64.39%). LCMS (ESI) [M+H]+=367.15; ¹H NMR (399 MHz, CD3OD) δ 8.07 (s, 1H), 7.73 (s, 1H), 6.93 (s, 1H), 6.59 (s, 1H), 6.54 (s, 1H), 4.54 (dd, J=26.1, 10.9 Hz, 4H), 4.04 (dd, J=26.3, 10.6 Hz, 2H), 3.89 (t, J=5.5 Hz, 2H), 3.78 (q, J=11.5 Hz, 2H), 3.60 (t, J=10.8 Hz, 1H), 2.41 (s, 2H), 1.27 (d, J=6.7 Hz, 3H).

### Example 9

### Preparation of (R)-3-methyl-4-(4-(6-methylpyridin-3-yl)-1-(1H-pyrazol-3-yl)-1H-pyrazolo[3,4-b]pyridin-6-yl)morpholine

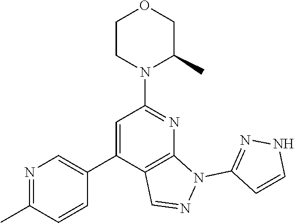

### Step 1: Preparation of (R)-3-methyl-4-(4-(6-methylpyridin-3-yl)-1-(1-((2-(trimethylsilyl)ethoxy)methyl)-1H-pyrazol-3-yl)-1H-pyrazole[3,4-b]pyridin-6-yl)morpholine

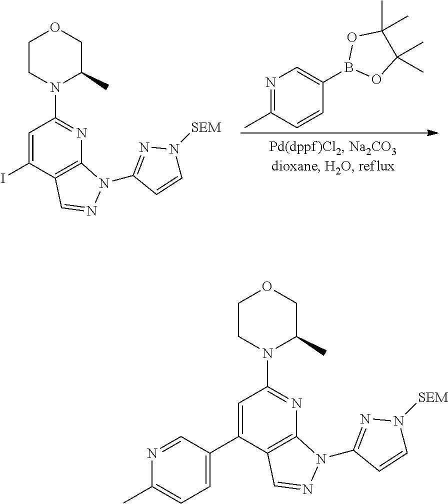

(R)-4-(4-Iodo-1-(1-((2-(trimethylsilyl)ethoxy)methyl)-1H-pyrazol-3-yl)-1H-pyrazole[3,4-b]pyridin-6-yl)-3-methylmorpholine (50 mg, 0.09 mmol, 1 equiv.), 2-methyl-5-(4,4,5,5-tetramethyl-1,3,2-dioxaborolan-2-yl)pyridine (15.2 mg, 0.11 mmol, 1.2 equiv.), sodium carbonate (19.61 mg, 0.19 mmol, 2 equiv.) and [1,1′-bis(diphenylphosphino)ferrocene]dichloropalladium (II) (6.79 mg, 0.01 mmol, 0.1 equiv.) were dissolved in 1,4-dioxane (2 mL) and water (1 mL), and the mixture was stirred at 90° C. for 2 hours. After the reaction was completed as monitored by LCMS, the reaction liquid was extracted with ethyl acetate and water, the organic phases were combined, dried, filtered and spun to dryness, and the residue was purified by thin layer chromatography (petroleum ether:ethyl acetate=1:1), to afford the target compound (38 mg, yield: 81.23%). LCMS (ESI) [M+H]⁺=506.45.

### Step 2: Preparation of (R)-3-methyl-4-(4-(6-methylpyridin-3-yl)-1-(1H-pyrazol-3-yl)-1H-pyrazolo[3,4-b]pyridin-6-yl)morpholine

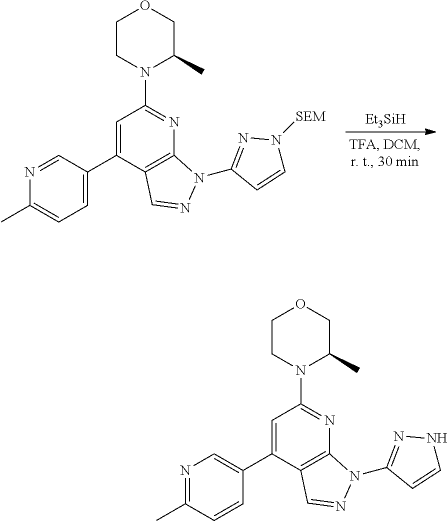 [SMILES: "Et3SiH" ; Cc1ccc(-c2cc(N3CCOC[C@H]3C)nc3c2cnn3-c2ccn(COCC[Si](C)(C)C)n2)cn1 ; Cc1ccc(-c2cc(N3CCOC[C@H]3C)nc3c2cnn3-c2cc[nH]n2)cn1]

(R)-3-Methyl-4-(4-(6-methylpyridin-3-yl)-1-(1-((2-(trimethylsilyl)ethoxy)methyl)-1H-pyrazol-3-yl)-1H-pyrazole[3,4-b]pyridin-6-yl)morpholine (38 mg, 0.08 mmol, 1 equiv.) was dissolved in trifluoroacetic acid (2 mL) and dichloromethane (2 mL), triethylsilane (0.2 mL) was added, and the mixture was stirred at room temperature for 30 minutes. After the reaction was completed as monitored by LCMS, the reaction liquid was adjusted to be alkaline with saturated sodium bicarbonate aqueous solution and extracted with ethyl acetate and water, the organic phases were combined, dried, filtered and concentrated, and the residue was purified by prepTLC (dichloromethane:methanol=10:1), to afford the target compound (12.1 mg, yield: 42.89%). LCMS (ESI) [M+H]⁺=376.15; ¹H NMR (399 MHz, DMSO-d6) δ 12.83 (s, 1H), 8.90 (s, 1H), 8.14 (d, J=7.7 Hz, 2H), 7.84 (s, 1H), 7.43 (d, J=7.8 Hz, 1H), 6.95 (s, 1H), 6.79 (s, 1H), 4.55 (s, 1H), 4.13 (d, J=13.2 Hz, 1H), 3.96 (d, J=10.7 Hz, 1H), 3.74 (d, J=11.7 Hz, 1H), 3.64 (d, J=11.4 Hz, 1H), 3.49 (t, J=11.4 Hz, 1H), 3.20 (d, J=12.2 Hz, 2H), 2.55 (s, 3H), 1.19 (d, J=6.0 Hz, 3H).

### Example 10

### (R)-6-((6-(3-Methylmorpholinyl)-1-(1H-pyrazolyl-5-yl)-1H-pyrazolyl[3,4-b]pyridin-4-yl)ethynyl)spiro[2.5]octan-6-ol

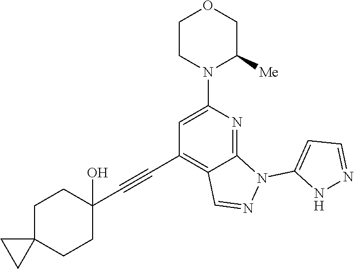

### Step 1: Preparation of 6-((trimethylsilyl)ethynyl)spiro[2.5]octan-6-ol

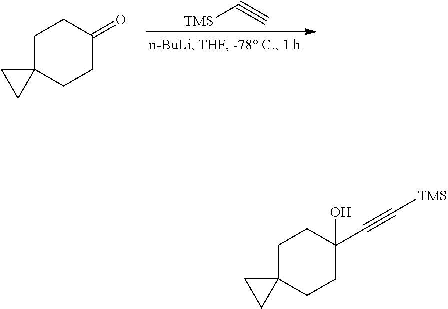

Spiro[2.5]octan-6-one (300 mg, 2.42 mmol, 1 equiv.) was dissolved in dry tetrahydrofuran (10 mL), and the reaction liquid was cooled to −78° C. under nitrogen protection. n-Butyllithium (1.45 mL, 3.63 mmol, 1.5 equiv., 2.5 M) was added dropwise, and the reaction liquid was stirred at −78° C. for 1 hour. Trimethylsilylacetylene (284.5 mg, 2.90 mmol, 1.2 equiv.) was added, and the reaction liquid was naturally warmed to room temperature and stirred for additional 1 hour. After the reaction was completed as monitored by TLC (potassium permanganate color developer), the reaction was quenched with saturated ammonium chloride aqueous solution (5 mL), the reaction liquid was extracted with water (5 mL) and ethyl acetate (5 mL×2), and the organic phase was dried and concentrated, to afford the target compound (380 mg, yield: 70.6%, crude).

### Step 2: Preparation of 6-ethylspiro[2.5]octan-6-ol

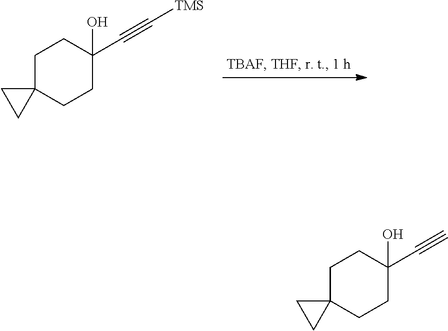

6-((Trimethylsilyl)ethynyl)spiro[2.5]octan-6-ol (380 mg, 1.71 mmol, 1 equiv.) was dissolved in tetrahydrofuran (5 mL), tetrabutylammonium fluoride (1.7 mL, 1.71 mmol, 1 equiv., 1 M) was added, and the reaction liquid was stirred at room temperature for 1 hour. After the reaction was completed as monitored by TLC (potassium permanganate color developer), the reaction liquid was extracted with ethyl acetate and water, and the organic phase was dried and concentrated, to afford the target compound (228 mg, yield: 88.8%, crude).

### Step 3: Preparation of (R)-6-((6-(3-methylmorpholinyl)-1-(1-((2-(trimethylsilyl)ethoxy)methyl)-1H-pyrazolyl-5-yl)-1H-pyrazolyl[3,4-b]pyridin-4-yl)ethynyl)spiro[2.5]octan-6-ol

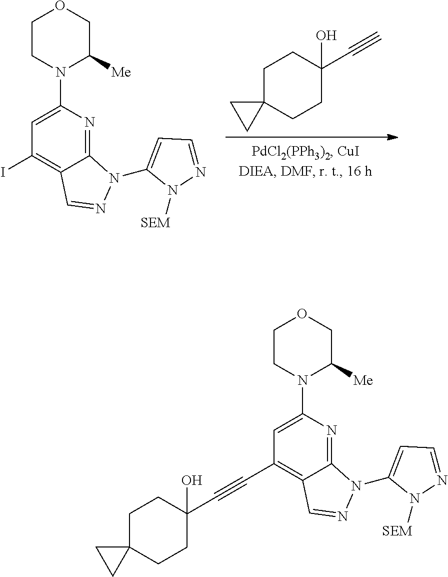 [SMILES: C[C@@H]1COCCN1c1cc(I)c2cnn(-c3ccnn3COCC[Si](C)(C)C)c2n1 ; C#CC1(O)CCC2(CC1)CC2 ; C[C@@H]1COCCN1c1cc(C#CC2(O)CCC3(CC2)CC3)c2cnn(-c3ccnn3COCC[Si](C)(C)C)c2n1]

(R)-4-(4-Iodo-1-(1-((2-(trimethylsilyl)ethoxy)methyl)-1H-pyrazol-5-yl)-1H-pyrazole[3,4-b]pyridin-6-yl)-3-methylmorpholine (50 mg, 0.09 mmol, 1 equiv.) was dissolved in N,N-dimethylformamide (2 mL), 6-ethylspiro[2.5]octan-6-ol (29 mg, 0.19 mmol, 2.1 equiv.), bis(triphenylphosphine)dichloropalladium (II) (2 mg, 0.003 mmol, 0.03 equiv.), cuprous iodide (1 mg, 0.005 mmol, 0.05 equiv.) and diisopropylethylamine (37 mg, 0.29 mmol, 3.1 equiv.) were added, and the reaction liquid was stirred at room temperature for 16 hours after nitrogen replacement was performed three times. After the reaction was completed as monitored by LCMS, the reaction liquid was diluted with water and extracted three times with ethyl acetate, the organic phases were combined, washed with saturated sodium chloride aqueous solution, dried and concentrated, and the residue was purified by thin layer chromatography (ethyl acetate:petroleum ether=1:3), to afford the target product (35 mg, yield: 69.1%). LCMS (ESI) [M+H]⁺=563.50.

### Step 4: Preparation of (R)-6-((6-(3-methylmorpholinyl)-1-(1H-pyrazolyl-5-yl)-1H-pyrazolyl[3,4-b]pyridin-4-yl)ethynyl)spiro[2.5]octan-6-ol

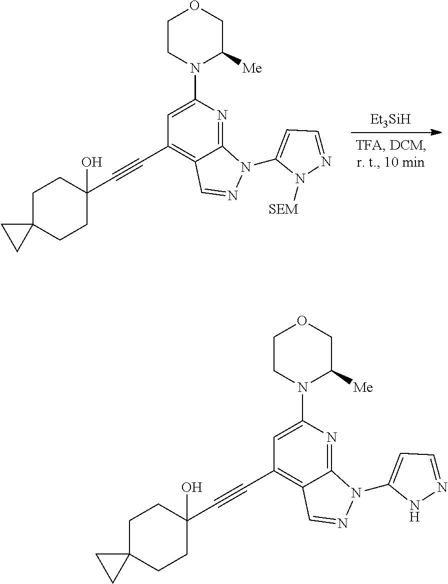 [SMILES: "Et3SiH" ; C[C@@H]1COCCN1c1cc(C#CC2(O)CCC3(CC2)CC3)c2cnn(-c3ccnn3COCC[Si](C)(C)C)c2n1 ; C[C@@H]1COCCN1c1cc(C#CC2(O)CCC3(CC2)CC3)c2cnn(-c3ccn[nH]3)c2n1]

(R)-6-((6-(3-Methylmorpholinyl)-1-(1-((2-(trimethylsilyl)ethoxy)methyl)-1H-pyrazolyl-5-yl)-1H-pyrazolyl[3,4-b]pyridin-4-yl)ethynyl)spiro[2.5]octan-6-ol (35 mg, 0.06 mmol, 1 equiv.) was dissolved in dichloromethane (0.6 mL), trifluoroacetic acid (0.8 mL) and triethylsilane (0.08 mL) were successively added, and the reaction liquid was stirred at room temperature for 10 minutes. After the reaction was completed as monitored by LCMS, the reaction liquid was adjusted to pH 9 with saturated sodium bicarbonate aqueous solution and then extracted three times with ethyl acetate, the organic phases were combined, washed with saturated sodium chloride aqueous solution, dried, filtered and concentrated, and the residue was purified by thin layer chromatography (dichloromethane:methanol=10:1), to afford the target compound (11.9 mg, yield: 45.8%). LCMS (ESI) [M+H]+=433.15; ¹H NMR (400 MHz, DMSO-d6) δ 12.83 (s, 1H), 7.96 (s, 1H), 7.82 (s, 1H), 6.82 (s, 1H), 6.75 (s, 1H), 5.67 (s, 1H), 4.43 (s, 1H), 4.03 (d, J=12.9 Hz, 1H), 3.93 (d, J=9.8 Hz, 1H), 3.71 (d, J=11.2 Hz, 1H), 3.60 (d, J=10.7 Hz, 1H), 3.45 (t, J=11.3 Hz, 1H), 3.14 (s, 1H), 1.96-1.90 (m, 2H), 1.73 (t, J=10.7 Hz, 2H), 1.59 (s, 2H), 1.24 (d, J=9.2 Hz, 2H), 1.16 (d, J=6.4 Hz, 3H), 0.31-0.26 (m, 2H), 0.23 (d, J=7.3 Hz, 2H).

### Example 11

### Preparation of (R)-4-(1-(1H-pyrazol-3-yl)-4-(1H-pyrrolyl[2,3-b]pyridin-4-yl)-1H-pyrazole[3,4-b]pyridin-6-yl)-3-methylmorpholine

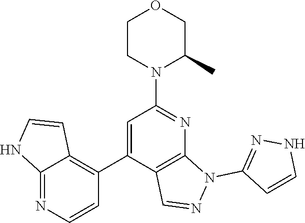

### Step 1: Preparation of (R)-4-(4-(1H-pyrrolyl[2,3-b]pyridin-4-yl)-1-(1-(2-(trimethylsilyl)ethoxy)methyl)-1H-pyrazol-3-yl)-1H-pyrazole[3,4-b]pyridin-6-yl)-3-methylmorpholine

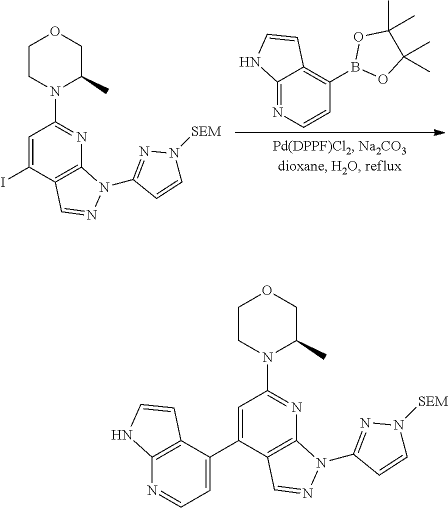 [SMILES: C[C@@H]1COCCN1c1cc(I)c2cnn(-c3ccn(COCC[Si](C)(C)C)n3)c2n1 ; CC1(C)OB(c2ccnc3[nH]ccc23)OC1(C)C ; C[C@@H]1COCCN1c1cc(-c2ccnc3[nH]ccc23)c2cnn(-c3ccn(COCC[Si](C)(C)C)n3)c2n1]

(R)-4-(4-Iodo-1-(1-((2-(trimethylsilyl)ethoxy)methyl)-1H-pyrazol-3-yl)-1H-pyrazole[3,4-b]pyridin-6-yl)-3-methylmorpholine (0 mg, 0.09 mmol, 1 equiv.), 4-(4,4,5,5-tetramethyl-1,3,2-dioxaborolan-2-yl)-1H-pyrrole[2,3-b]pyridine (27 mg, 0.11 mmol, 1.2 equiv.), [1,1′-bis(diphenylphosphino)ferrocene]dichloropalladium (II) (6.8 mg, 0.001 mmol, 0.01 equiv.) and sodium carbonate (19.7 mg, 0.19 mmol, 2.0 equiv.) were dissolved in 1,4-dioxane (2 ml), water (2 mL) was added dropwise, and the mixture was stirred at room temperature for 2 hours. After the reaction was completed as monitored by LCMS, the reaction liquid was extracted with ethyl acetate and water, the organic phases were combined, dried, filtered and spun to dryness, and the residue was purified by prepTLC (petroleum ether:ethyl acetate=1:1), to afford the target compound (24 mg, yield: 50.25%). LCMS (ESI) [M+H]⁺=531.20.

### Step 2: Preparation of (R)-4-(1-(1H-pyrazol-3-yl)-4-(1H-pyrrolyl[2,3-b]pyridin-4-yl)-1H-pyrazole[3,4-b]pyridin-6-yl)-3-methylmorpholine

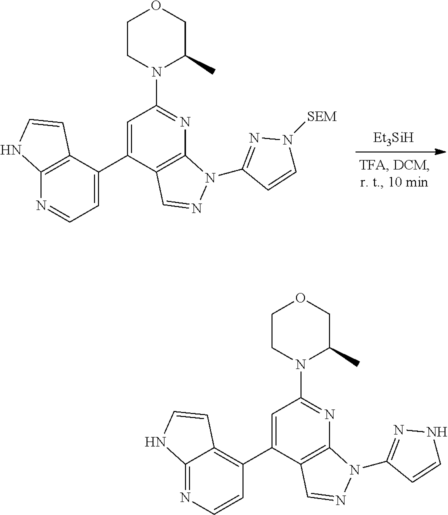 [SMILES: "Et3SiH" ; C[C@@H]1COCCN1c1cc(-c2ccnc3[nH]ccc23)c2cnn(-c3ccn(COCC[Si](C)(C)C)n3)c2n1 ; C[C@@H]1COCCN1c1cc(-c2ccnc3[nH]ccc23)c2cnn(-c3cc[nH]n3)c2n1]

((R)-4-(4-(1H-Pyrrolyl[2,3-b]pyridin-4-yl)-1-(1-(2-(trimethylsilyl)ethoxy)methyl)-1H-pyrazol-3-yl)-1H-pyrazole[3,4-b]pyridin-6-yl)-3-methylmorpholine (24 mg, 0.04 mmol, 1 equiv.) was dissolved in trifluoroacetic acid (1 mL) and dichloromethane (1 mL), triethylsilane (0.1 mL) was added, and the mixture was stirred at room temperature for 10 minutes. After the reaction was completed as monitored by LCMS, the reaction liquid was adjusted to be alkaline with saturated sodium bicarbonate aqueous solution and extracted with ethyl acetate and water, the organic phases were combined, dried, filtered and concentrated, and the residue was purified by prepTLC (dichloromethane:methanol=10:1), to afford the target compound (7.8 mg, yield: 38.96%). LCMS (ESI) [M+H]+=401.10; ¹H NMR (399 MHz, CD3OD) δ 8.35 (d, J=5.4 Hz, 1H), 7.93 (s, 1H), 7.77 (s, 1H), 7.50 (d, J=3.5 Hz, 1H), 7.38 (d, J=4.8 Hz, 1H), 7.00 (d, J=7.2 Hz, 2H), 6.55 (d, J=3.2 Hz, 1H), 4.57 (s, 1H), 4.17 (d, J=13.6 Hz, 1H), 4.02 (s, 1H), 3.82 (d, J=4.1 Hz, 2H), 3.65 (s, 1H), 3.37 (s, 1H), 1.35 (d, J=6.6 Hz, 3H).

### Example 12

### Preparation of (R)-1-(3-fluorophenyl)-4-((6-(3-methylmorpholinyl)-1-(1H-pyrazolyl-5-yl)-1H-pyrazolyl[3,4-b]pyridin-4-yl)ethynyl)piperidin-4-ol

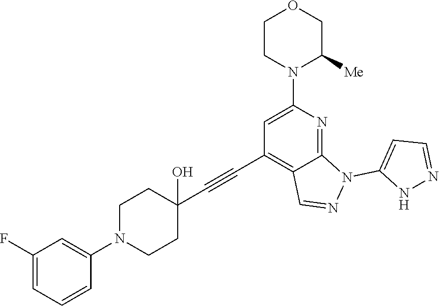

### Step 1: Preparation of 1-(3-fluorophenyl)-4-((trimethylsilyl)ethynyl)piperidin-4-ol

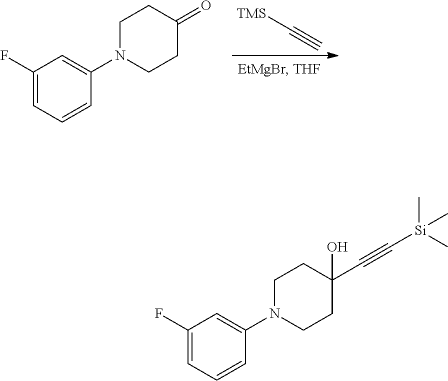

Trimethylsilylacetylene (381.25 mg, 3.88 mmol, 1.5 equiv.) was added to tetrahydrofuran (5 mL) at room temperature, and the mixture was cooled to −78° C. after nitrogen replacement was performed three times. Subsequently, n-butyllithium (207.21 mg, 3.23 mmol, 1.25 equiv.) was injected into the reaction system, and the resulting mixture was stirred at −78° C. for additional 1 hour. 1-(3-Fluorophenyl)piperidin-4-one (500 mg, 2.59 mmol, 1 equiv.) was dissolved in tetrahydrofuran solution (1 mL) and slowly injected into the reaction system, and the mixture was stirred at −78° C. for 0.5 hours, then slowly returned to room temperature and stirred for 30 minutes. After the reaction was completed as monitored by LCMS, the reaction was quenched with saturated ammonium chloride aqueous solution, and the reaction liquid was extracted three times with ethyl acetate. The organic phases were combined, washed with saturated sodium chloride aqueous solution, dried, filtered and concentrated, and the concentrate was purified by thin layer chromatography, to afford the target compound (310 mg, yield: 41.07%). LCMS (ESI) [M+H]+=292.44.

### Step 2: Preparation of 4-ethynyl-1-(3-fluorophenyl)piperidin-4-ol

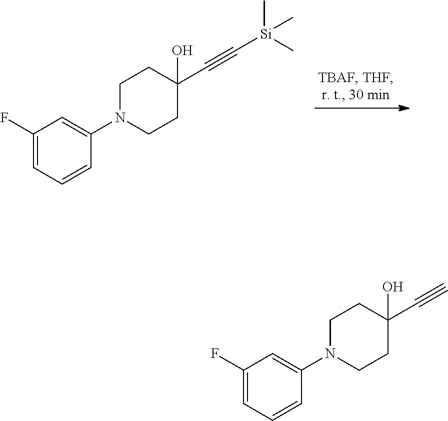 [SMILES: C[Si](C)(C)C#CC1(O)CCN(c2cccc(F)c2)CC1 ; C#CC1(O)CCN(c2cccc(F)c2)CC1]

1-(3-Fluorophenyl)-4-((trimethylsilyl)ethynyl)piperidin-4-ol (20 mg, 0.07 mmol, 1 equiv.) was dissolved in tetrahydrofuran (10 mL) at room temperature, then tetrabutylammonium fluoride (1 ml, 1 mmol, 14.57 equiv.) was slowly added to the reaction system, and the mixture was placed at room temperature for 30 minutes after nitrogen replacement was performed three times. After the reaction was completed as monitored by LCMS, the reaction liquid was diluted with water and extracted three times with ethyl acetate, and the organic phases were combined, washed with saturated sodium chloride aqueous solution, dried, filtered and concentrated, to afford the crude target compound (15 mg, yield: 97.74%). LCMS (ESI) [M+H]⁺=220.25.

### Step 3: Preparation of (R)-1-(3-fluorophenyl)-4-((6-(3-methylmorpholinyl)-1-(1-((2-(trimethylsilyl)ethoxy)methyl)-1H-pyrazol-5-yl)-1H-pyrazolyl[3,4-b]pyridin-4-yl)ethynyl)piperidin-4-ol

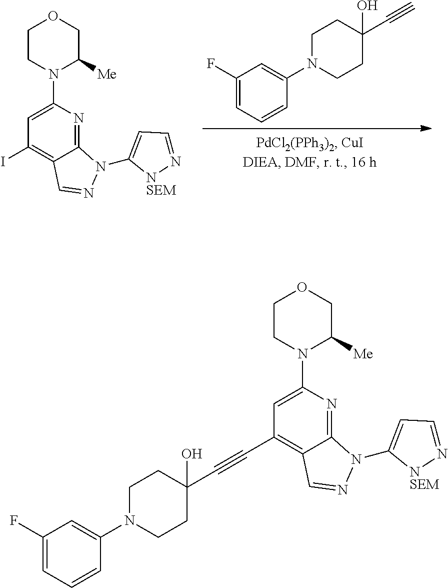 [SMILES: C[C@@H]1COCCN1c1cc(I)c2cnn(-c3ccnn3COCC[Si](C)(C)C)c2n1 ; C#CC1(O)CCN(c2cccc(F)c2)CC1 ; C[C@@H]1COCCN1c1cc(C#CC2(O)CCN(c3cccc(F)c3)CC2)c2cnn(-c3ccnn3COCC[Si](C)(C)C)c2n1]

(R)-4-(4-Iodo-1-(1-((2-(trimethylsilyl)ethoxy)methyl)-1H-pyrazol-5-yl)-1H-pyrazole[3,4-b]pyridin-6-yl)-3-methylmorpholine (175 mg, 0.32 mmol, 1 equiv.), 4-ethynyl-1-(3-fluorophenyl)piperidin-4-ol (149.08 mg, 0.68 mmol, 2.1 equiv.), cuprous iodide (3.08 mg, 0.02 mmol, 0.05 equiv.), N,N-diisopropylethylamine (133.91 mg, 1.04 mmol, 3.2 equiv.) and bis(triphenylphosphine)dichloropalladium (II) (6.82 mg, 0.01 mmol, 0.03 equiv.) were added to N,N-dimethylformamide (10 mL) at room temperature, and the mixture was stirred at room temperature for 16 hours after nitrogen replacement was performed three times. After the reaction was completed as monitored by LCMS, the reaction liquid was filtered, the filtrate was diluted with water and extracted three times with ethyl acetate, the organic phases were combined, washed with saturated sodium chloride aqueous solution, dried, filtered and concentrated, and the concentrate was purified by thin layer chromatography (petroleum ether:ethyl acetate=1:1), to afford the target compound (120 mg, yield: 58.66%). LCMS (ESI) [M+H]⁺=632.82.

### Step 4: Preparation of (R)-1-(3-fluorophenyl)-4-((6-(3-methylmorpholinyl)-1-(1H-pyrazolyl-5-yl)-1H-pyrazolyl[3,4-b]pyridin-4-yl)ethynyl)piperidin-4-ol

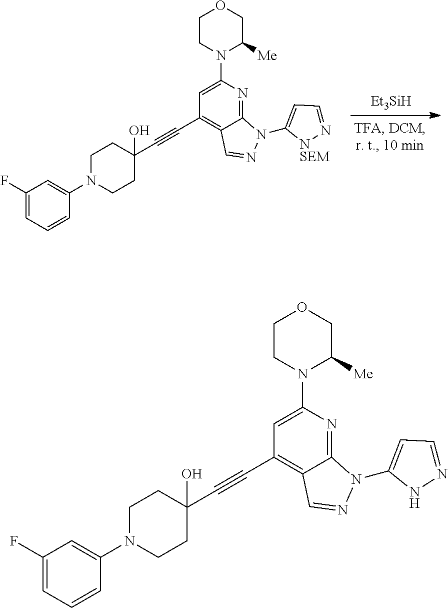 [SMILES: "Et3SiH" ; C[C@@H]1COCCN1c1cc(C#CC2(O)CCN(c3cccc(F)c3)CC2)c2cnn(-c3ccnn3COCC[Si](C)(C)C)c2n1 ; C[C@@H]1COCCN1c1cc(C#CC2(O)CCN(c3cccc(F)c3)CC2)c2cnn(-c3ccn[nH]3)c2n1]

(R)-1-(3-Fluorophenyl)-4-((6-(3-methylmorpholinyl)-1-(1-((2-(trimethylsilyl)ethoxy)methyl)-1H-pyrazol-5-yl)-1H-pyrazolyl[3,4-b]pyridin-4-yl)ethynyl)piperidin-4-ol (120 mg, 0.19 mmol, 1 equiv.) was added to a mixed solvent of trifluoroacetic acid (2 mL) and dichloromethane (2 mL) at room temperature, then triethylsilane (0.2 ml, 1.72 mmol, 9.06 equiv.) was slowly added to the reaction system, and the mixture was reacted at room temperature for 10 minutes after nitrogen replacement was performed three times. After the reaction was completed as monitored by LCMS, the reaction liquid was adjusted to pH 9 with saturated sodium bicarbonate aqueous solution and then extracted three times with ethyl acetate, the organic phases were combined, washed with saturated sodium chloride aqueous solution, dried, filtered and concentrated, and the concentrate was purified by thin layer chromatography (dichloromethane:methanol=10:1), to afford the target compound (34.4 mg, yield: 36.1%). LCMS (ESI) [M+H]+=502.56; ¹H NMR (400 MHz, DMSO-d6) δ 12.83 (s, 1H), 7.98 (s, 1H), 7.81 (s, 1H), 7.18 (d, J=7.5 Hz, 1H), 6.84 (s, 1H), 6.75 (d, J=11.5 Hz, 3H), 6.49 (s, 1H), 5.95 (s, 1H), 4.43 (s, 1H), 4.00 (s, 1H), 3.91 (s, 1H), 3.70 (d, J=11.2 Hz, 1H), 3.61 (s, 1H), 3.51 (s, 2H), 3.44 (s, 4H), 3.20 (s, 7H), 2.00 (s, 2H), 1.83 (s, 2H), 1.15 (d, J=6.6 Hz, 3H).

### Example 13

### Preparation of (3R)-4-(1-(1H-pyrazol-3-yl)-4-(tetrahydro-2H-pyran-3-yl)-1H-pyrazole[3,4-b]pyridin-6-yl)-3-methylmorpholine

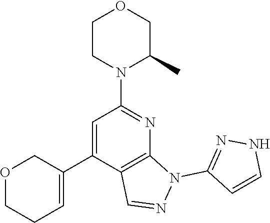

### Step 1: Preparation of (3R)-4-(1-(1H-pyrazol-3-yl)-4-(tetrahydro-2H-pyran-3-yl)-1H-pyrazole[3,4-b]pyridin-6-yl)-3-methylmorpholine

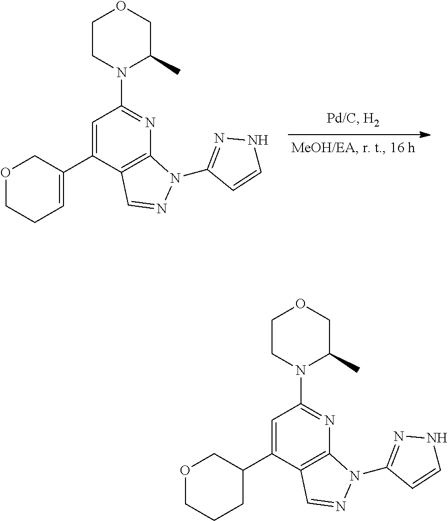 [SMILES: C[C@@H]1COCCN1c1cc(C2=CCCOC2)c2cnn(-c3cc[nH]n3)c2n1 ; C[C@@H]1COCCN1c1cc(C2CCCOC2)c2cnn(-c3cc[nH]n3)c2n1]

(R)-4-(4,6-Dihydro-2H-pyran-3-yl)-1-(1H-pyrazol-3-yl)-1H-pyrazole[3,4-b]pyridin-6-yl)-3-methylmorpholine (50 mg, 0.14 mmol, 1 equiv.) was dissolved in methanol (5 mL) and ethyl acetate (1 mL), palladium on carbon (10 mg, 0.09 mmol, 0.67 equiv.) was added, and the mixture was stirred overnight at room temperature after hydrogen replacement was performed several times. After the reaction was completed as monitored by LCMS, the palladium on carbon in the reaction liquid was filtered off, the filtrate was spun to dryness, and the residue was purified by thin layer chromatography (dichloromethane:methanol=10:1), to afford the target compound (36 mg, yield: 69.79%). LCMS (ESI) [M+H]+=369.20; ¹H NMR (399 MHz, DMSO-d6) δ 8.20 (s, 1H), 7.77 (s, 1H), 6.70 (d, J=31.5 Hz, 2H), 4.43 (s, 1H), 4.02 (d, J=12.9 Hz, 1H), 3.88 (d, J=10.3 Hz, 3H), 3.72 (d, J=11.4 Hz, 1H), 3.60 (d, J=11.3 Hz, 2H), 3.47 (d, J=10.8 Hz, 2H), 3.13 (d, J=10.5 Hz, 2H), 1.96 (s, 2H), 1.67 (s, 2H), 1.15 (d, J=5.2 Hz, 3H).

### Example 14

### Preparation of (R)-9-((6-(3-methylmorpholinyl)-1-(1H-pyrazol-5-yl)-1H-pyrazole[3,4-b]pyridin-4-yl)ethynyl)-3-oxaspiro[5.5]undecan-9-ol

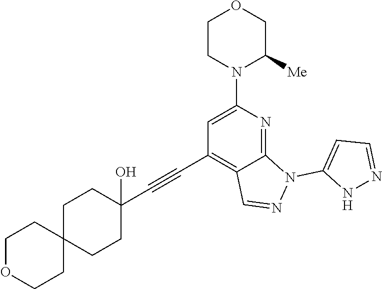

### Step 1: Preparation of 9-((trimethylsilyl)ethynyl)-3-oxaspiro[5.5]undecan-9-ol

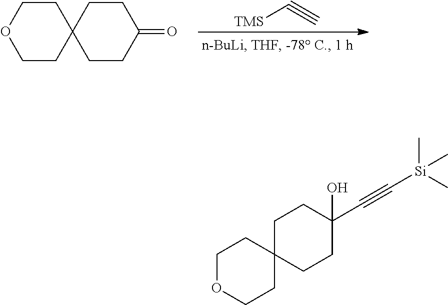

Trimethylsilylacetylene (241.2 mg, 2.46 mmol, 1.5 equiv.) was added to tetrahydrofuran (5 mL) at room temperature, and the mixture was cooled to −78° C. after nitrogen replacement was performed three times. Subsequently, n-butyllithium (131.09 mg, 2.05 mmol, 1.25 equiv.) was injected into the reaction system, and the resulting mixture was stirred at −78° C. for additional 1 hour. 3-Hydroxamic acid [5.5]undecan-9-one (300 mg, 1.64 mmol, 1 equiv.) was dissolved in tetrahydrofuran (1 mL) and slowly injected into the reaction system, and the mixture was stirred at −78° C. for 0.5 hours, then slowly returned to room temperature and stirred for 30 minutes. After the reaction was completed as monitored by LCMS, the reaction was quenched with saturated ammonium chloride aqueous solution, and the reaction liquid was extracted three times with ethyl acetate. The organic phases were combined, washed with saturated sodium chloride aqueous solution, dried, filtered and concentrated, and the concentrate was purified by thin layer chromatography, to afford the target compound (300 mg, yield: 64.99%). LCMS (ESI) [M+H]+=267.45.

### Step 2: Preparation of 9-ethynyl-3-oxaspiro[5.5]undecan-9-ol

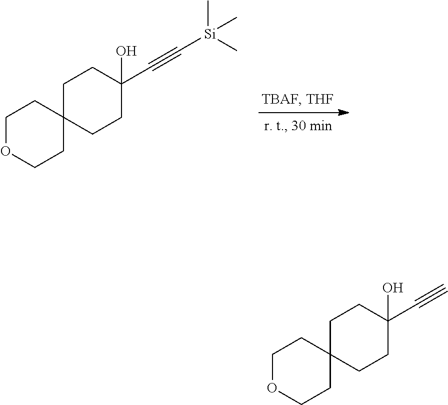 [SMILES: C[Si](C)(C)C#CC1(O)CCC2(CCOCC2)CC1 ; C#CC1(O)CCC2(CCOCC2)CC1]

9-((Trimethylsilyl)ethynyl)-3-oxaspiro[5.5]undecan-9-ol (310 mg, 1.06 mmol, 1 equiv.) was added to tetrahydrofuran (10 mL) at room temperature, then tetrabutylammonium fluoride (2 mL, 2 mmol, 1.88 equiv.) was slowly added to the reaction system, and the mixture was stirred at room temperature for 30 minutes after nitrogen replacement was performed three times. After the reaction was completed as monitored by LCMS, the reaction liquid was diluted with water and extracted three times with ethyl acetate, and the organic phases were combined, washed with saturated sodium chloride aqueous solution, dried, filtered and concentrated, to afford the crude target compound (230 mg, yield: 98.97%). LCMS (ESI) [M+H]⁺=195.27.

### Step 3: Preparation of (R)-9-((6-(3-methylmorpholinyl)-1-(1-((2-(trimethylsilyl)ethoxy)methyl)-1H-pyrazol-5-yl)-1H-pyrazole[3,4-b]pyridin-4-yl)ethynyl)-3-oxaspiro[5.5]undecan-9-ol

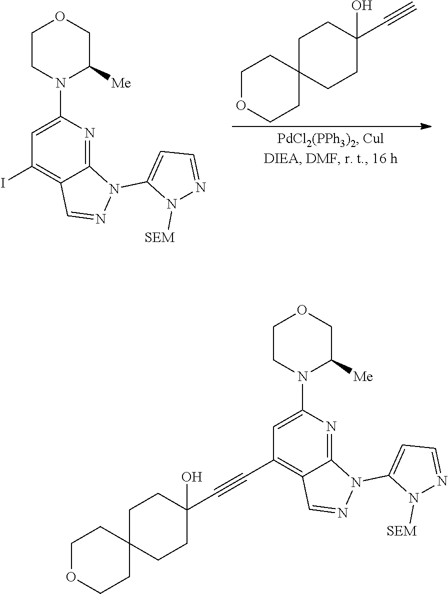 [SMILES: C[C@@H]1COCCN1c1cc(I)c2cnn(-c3ccnn3COCC[Si](C)(C)C)c2n1 ; C#CC1(O)CCC2(CCOCC2)CC1 ; C[C@@H]1COCCN1c1cc(C#CC2(O)CCC3(CCOCC3)CC2)c2cnn(-c3ccnn3COCC[Si](C)(C)C)c2n1]

(R)-4-(4-Iodo-1-(1-((2-(trimethylsilyl)ethoxy)methyl)-1H-pyrazol-5-yl)-1H-pyrazole[3,4-b]pyridin-6-yl)-3-methylmorpholine (55 mg, 0.1 mmol, 1 equiv.), 9-ethynyl-3-oxaspiro[5.5]undecan-9-ol (41.52 mg, 0.21 mmol, 2.1 equiv.), cuprous iodide (0.97 mg, 0.01 mmol, 0.05 equiv.), N,N-diisopropylethylamine (42.09 mg, 0.33 mmol, 3.2 equiv.) and bis(triphenylphosphine)dichloropalladium (II) (2.14 mg, 0.003 mmol, 0.03 equiv.) were added to N,N-dimethylformamide (10 mL) at room temperature, and the mixture was stirred at room temperature for additional 16 hours after nitrogen replacement was performed three times. After the reaction was completed as monitored by LCMS, the reaction liquid was filtered, the filtrate was diluted with water and extracted three times with ethyl acetate, the organic phases were combined, washed with saturated sodium chloride aqueous solution, dried, filtered and concentrated, and the concentrate was purified by thin layer chromatography (petroleum ether:ethyl acetate=1:1), to afford the target product (25 mg, yield: 52.46%). LCMS (ESI) [M+H]⁺=607.83

### Step 4: Preparation of (R)-9-((6-(3-methylmorpholinyl)-1-(1H-pyrazol-5-yl)-1H-pyrazole[3,4-b]pyridin-4-yl)ethynyl)-3-oxaspiro[5.5]undecan-9-ol

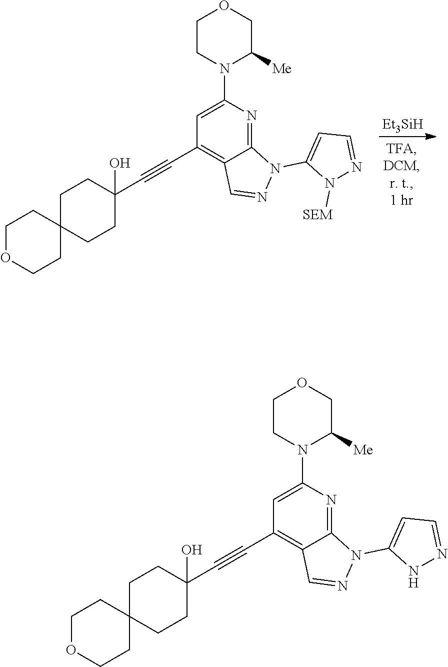 [SMILES: "Et3SiH" ; C[C@@H]1COCCN1c1cc(C#CC2(O)CCC3(CCOCC3)CC2)c2cnn(-c3ccnn3COCC[Si](C)(C)C)c2n1 ; C[C@@H]1COCCN1c1cc(C#CC2(O)CCC3(CCOCC3)CC2)c2cnn(-c3ccn[nH]3)c2n1]

(R)-9-((6-(3-Methylmorpholinyl)-1-(1-((2-(trimethylsilyl)ethoxy)methyl)-1H-pyrazol-5-yl)-1H-pyrazole[3,4-b]pyridin-4-yl)ethynyl)-3-oxaspiro[5.5]undecan-9-ol (25 mg, 0.04 mmol, 1 equiv.) was added to a mixed solvent of trifluoroacetic acid (2 mL) and dichloromethane (2 mL) at room temperature, then triethylsilane (0.2 ml, 1.72 mmol, 41.75 equiv.) was slowly added to the reaction system, and the mixture was reacted at room temperature for 1 hour after nitrogen replacement was performed three times. After the reaction was completed as monitored by LCMS, the reaction liquid was adjusted to pH 9 with saturated sodium bicarbonate aqueous solution and then extracted three times with ethyl acetate, the organic phases were combined, washed with saturated sodium chloride aqueous solution, dried, filtered and concentrated, and the concentrate was purified by thin layer chromatography (dichloromethane:methanol=10:1), to afford the target compound (4.8 mg, yield: 25.18%). LCMS (ESI) [M+H]+=477.57; ¹H NMR (400 MHz, DMSO-d6) δ 12.82 (s, 1H), 7.95 (s, 1H), 7.82 (s, 1H), 6.79 (s, 1H), 6.74 (s, 1H), 5.62 (s, 1H), 4.42 (s, 1H), 4.03 (d, J=13.8 Hz, 1H), 3.92 (d, J=10.6 Hz, 1H), 3.71 (d, J=11.2 Hz, 1H), 3.60 (d, J=13.1 Hz, 1H), 3.52 (s, 4H), 3.45 (s, 1H), 3.13 (s, 1H), 1.81 (s, 2H), 1.69 (d, J=11.1 Hz, 4H), 1.45 (s, 2H), 1.39 (s, 4H), 1.15 (d, J=6.6 Hz, 3H).

### Example 15

### Preparation of (R)-6-(4-(6-(3-methylmorpholinyl)-1-(1H-pyrazolyl-3-yl)-1H-pyrazolyl[3,4-b]pyridin-4-yl)phenyl)-2-oxo-6-azaspiro[3.3]heptane

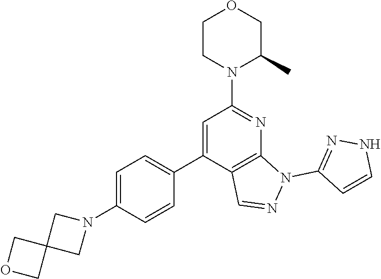

### Step 1: Preparation of ((R)-4-(4-(4-bromophenyl)-1-(1-(2-(trimethylsilyl)ethoxy)methyl)-1H-pyrazol-5-yl)-1H-pyrazole[3,4-b]pyridin-6-yl)-3-methylmorpholine

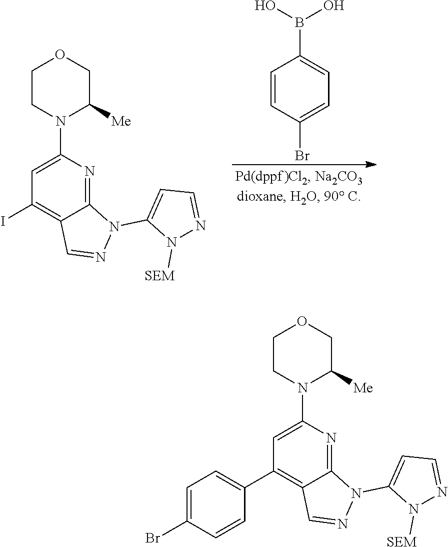 [SMILES: C[C@@H]1COCCN1c1cc(I)c2cnn(-c3ccnn3COCC[Si](C)(C)C)c2n1 ; OB(O)c1ccc(Br)cc1 ; C[C@@H]1COCCN1c1cc(-c2ccc(Br)cc2)c2cnn(-c3ccnn3COCC[Si](C)(C)C)c2n1]

p-Bromophenylboronic acid (41 mg, 0.204 mmol, 1.1 equiv.), (R)-4-(4-iodo-1-(1-((2-(trimethylsilyl)ethoxy)methyl)-1H-pyrazol-5-yl)-1H-pyrazole[3,4-b]pyridin-6-yl)-3-methylmorpholine (100 mg, 0.185 mmol, 1.0 equiv.), [1,1′-bis(diphenylphosphino)ferrocene]dichloropalladium (II) (14 mg, 0.018 mmol, 0.1 equiv) and sodium carbonate (40 mg, 0.37 mmol, 2.0 equiv.) were weighed and dissolved in 1,4-dioxane (4 mL) and water (2 mL), and then the mixture was heated to 90° C. and reacted at this temperature for 3 hours after nitrogen replacement was performed three times. After the raw materials were reacted completely as monitored by TLC and a product was generated as monitored by LCMS, the reaction liquid was filtered and then extracted with ethyl acetate (20 mL×3), the organic phases were combined and spun to dryness, and the residue was purified by thin layer chromatography (petroleum ether:ethyl acetate=3:1), to afford the target compound (50 mg, yield: 43.1%). LCMS (ESI) [M+H]+=569.15

### Step 2: Preparation of (R)-6-(4-(6-(3-methylmorpholinyl)-1-(1-(2-(trimethylsilyl)ethoxy)methyl)-1H-pyrazol-5-yl)-1H-pyrazole[3,4-b]pyridin-4-yl)phenyl)-2-oxo-6-azaspiro[3.3]heptane

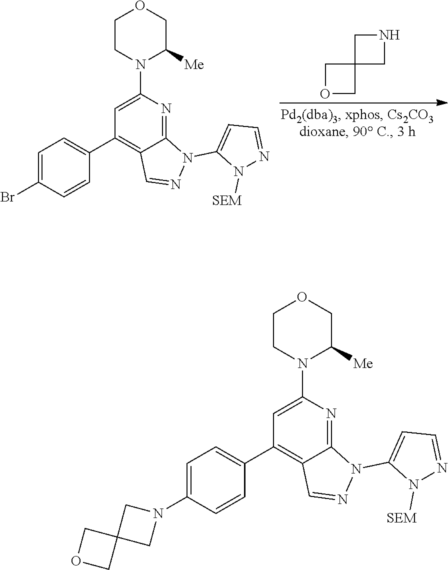 [SMILES: C[C@@H]1COCCN1c1cc(-c2ccc(Br)cc2)c2cnn(-c3ccnn3COCC[Si](C)(C)C)c2n1 ; C1NCC12COC2 ; C[C@@H]1COCCN1c1cc(-c2ccc(N3CC4(COC4)C3)cc2)c2cnn(-c3ccnn3COCC[Si](C)(C)C)c2n1]

((R)-4-(4-(4-Bromophenyl)-1-(1-(2-(trimethylsilyl)ethoxy)methyl)-1H-pyrazol-5-yl)-1H-pyrazole[3,4-b]pyridin-6-yl)-3-methylmorpholine (50 mg, 0.11 mmol, 1.0 equiv.), 2-oxo-6-azaspiro[3.3]heptane (12 mg, 0.117 mmol, 1.1 equiv.), tris(dibenzylideneacetone)dipalladium (20 mg, 0.022 mmol, 0.2 equiv), 2-dicyclohexylphosphino-2′,4′,6′-triisopropylbiphenyl (6 mg, 0.011 mmol, 0.1 equiv) and cesium carbonate (72 mg, 0.22 mmol, 2.0 equiv) were dissolved in 1,4-dioxane (5 mL), and the mixture was reacted at 90° C. for 3 hours after nitrogen replacement was performed three times. After the raw materials were reacted completely as monitored by TLC and a product was generated as monitored by LCMS, the reaction liquid was filtered and then extracted with ethyl acetate (20 mL×3), the organic phases were combined and spun to dryness, and the residue was purified by prepTLC (petroleum ether:ethyl acetate=1:1), to afford the product (20 mg, yield: 39.2%). LCMS (ESI) [M+H]+=588.30.

### Step 3: Preparation of (R)-6-(4-(6-(3-methylmorpholinyl)-1-(1H-pyrazolyl-3-yl)-1H-pyrazolyl[3,4-b]pyridin-4-yl)phenyl)-2-oxo-6-azaspiro[3.3]heptane

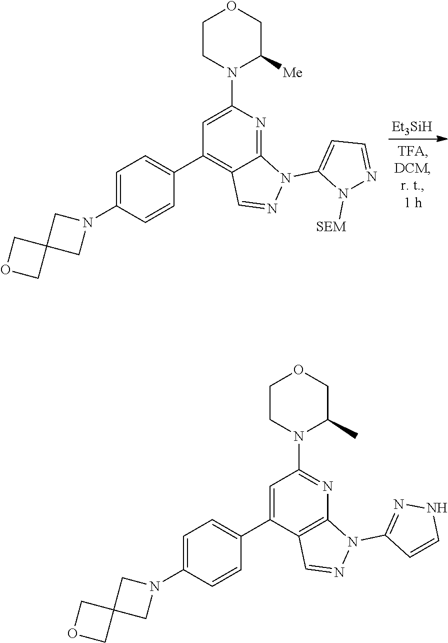 [SMILES: "Et3SiH" ; C[C@@H]1COCCN1c1cc(-c2ccc(N3CC4(COC4)C3)cc2)c2cnn(-c3ccnn3COCC[Si](C)(C)C)c2n1 ; C[C@@H]1COCCN1c1cc(-c2ccc(N3CC4(COC4)C3)cc2)c2cnn(-c3cc[nH]n3)c2n1]

(R)-6-(4-(6-(3-Methylmorpholinyl)-1-(1-(2-(trimethylsilyl)ethoxy)methyl)-1H-pyrazol-5-yl)-1H-pyrazole[3,4-b]pyridin-4-yl)phenyl)-2-oxo-6-azaspiro[3.3]heptane (20 mg, 0.034 mmol, 1.0 equiv.) was dissolved in dichloromethane (2 mL), then trifluoroacetic acid (2 mL) and triethylsilane (0.2 mL) were added, and then the mixture was reacted at 25° C. for 1 hour after nitrogen replacement was performed three times. After the raw materials were reacted completely as monitored by TLC and a product was generated as monitored by LCMS, the reaction was quenched with saturated sodium bicarbonate (50 mL), and then the reaction liquid was extracted with ethyl acetate (20 mL×3). The organic phases were combined and spun to dryness, and the residue was purified by thin layer chromatography (dichloromethane/methanol=10:1), to afford the product (5.9 mg, yield: 37.8%). LCMS (ESI) [M+H]+=458.20; ¹H NMR (400 MHz, DMSO-d6) δ 12.78 (s, 1H), 8.10 (s, 1H), 7.82 (s, 1H), 7.69 (d, J=8.7 Hz, 2H), 6.78 (d, J=8.6 Hz, 2H), 6.57 (d, J=8.6 Hz, 2H), 4.72 (s, 4H), 4.52 (s, 1H), 4.13-4.03 (m, 6H), 3.95 (d, J=10.9 Hz, 1H), 3.74 (d, J=11.4 Hz, 1H), 3.64 (d, J=11.1 Hz, 1H), 3.48 (s, 1H), 1.18 (d, J=6.3 Hz, 3H).

### Example 16

### (R)-2-Methyl-4-(6-(3-methylmorpholine)-1-(1H-pyrazol-3-yl)-1H-pyrazolo[3,4-b]pyridin-4-yl)3-methylbutynol

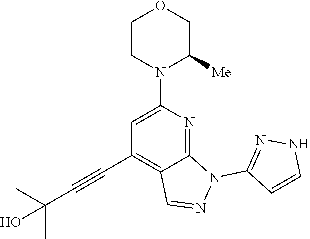

### Step 1: (R)-2-Methyl-4-(6-(3-methylmorpholinyl)-1-(1-((2-(trimethylsilyl)ethoxy)methyl)-1H-pyrazol-3-yl)-1H-pyrazole[3,4-b]pyridin-4-yl)3-methylbutynol

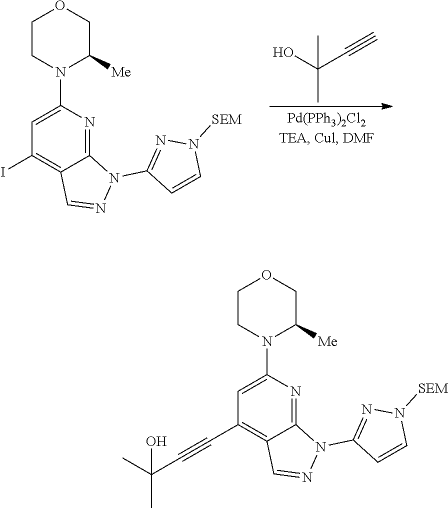

(R)-4-(4-Iodo-1-(1-((2-(trimethylsilyl)ethoxy)methyl)-1H-pyrazol-3-yl)-1H-pyrazole[3,4-b]pyridin-6-yl)-3-methylmorpholine (100.2 mg, 0.19 mmol, 1 equiv.), 3-methylbutynol-3 (17.1 mg, 0.20 mmol, 1.1 equiv.) and triethylamine (37.5 mg, 0.37 mmol, 2 equiv.) were weighed and dissolved in DMF (4.0 ml), then CuI (3.5 mg, 0.02 mmol, 0.1 equiv.) and Pd(PPh₃)₂Cl₂ (13.0 mg, 0.02 mmol, 0.1 equiv.) were weighed, and the mixture was heated to 40° C. and reacted for 1 h after gas replacement was performed under nitrogen protection. After the raw materials were consumed completely as monitored by TLC, the reaction liquid was cooled to room temperature (25° C.), 15 ml of water was added to the reaction liquid, and then 15 ml of EA was added to perform extraction and separation. The aqueous phase was extracted with EA (15 ml) to the point where no product remained in the aqueous phase, and the organic phases were combined, washed once with 20 ml of water, then washed once with 15 ml of saturated sodium chloride aqueous solution, dried, mixed, and purified by column chromatography (PE/EA=3:1), to afford the product (70.0 mg). For TLC, PE:EA=3:1. The product Rf value was (0.3). LCMS: [M+H]⁺=537.43.

### Step 2: (R)-2-Methyl-4-(6-(3-methylmorpholine)-1-(1H-pyrazol-3-yl)-1H-pyrazolo[3,4-b]pyridin-4-yl)3-methylbutynol

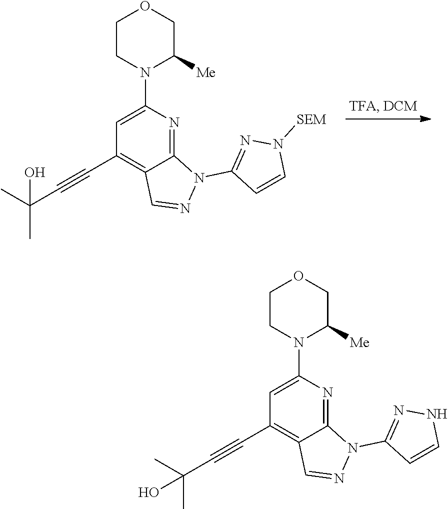 [SMILES: C[C@@H]1COCCN1c1cc(C#CC(C)(C)O)c2cnn(-c3ccn(COCC[Si](C)(C)C)n3)c2n1 ; C[C@@H]1COCCN1c1cc(C#CC(C)(C)O)c2cnn(-c3cc[nH]n3)c2n1]

(R)-2-Methyl-4-(6-(3-methylmorpholinyl)-1-(1-((2-(trimethylsilyl)ethoxy)methyl)-1H-pyrazol-3-yl)-1H-pyrazole[3,4-b]pyridin-4-yl)3-methylbutynol (70.0 mg, 0.14 mmol, 1 equiv.) was dissolved in dichloromethane (5.0 mL), TFA (1.0 ml) was added dropwise, and the mixture was stirred at room temperature (25° C.) for 5 hours. After the reaction was completed as monitored by LCMS, the reaction liquid was adjusted to pH=8 with saturated NaHCO₃ aqueous solution and extracted with DCM, and the organic phase was concentrated to dryness, separated by acetonitrile/water (0.1% NH4HCO3) and lyophilized, to afford the target compound (15 mg, purity: 90.5%, yield: 27%). LCMS: [M+H]⁺=367.34; ¹H NMR (400 MHz, DMSO) δ 12.86 (s, 1H), 8.03 (s, 1H), 7.84 (s, 1H), 6.83 (s, 1H), 6.78 (s, 1H), 5.68 (s, 1H), 4.45 (d, J=4.8 Hz, 1H), 4.07 (d, J=12.3 Hz, 1H), 4.00-3.91 (m, 1H), 3.74 (d, J=3.5 Hz, 1H), 3.64 (dd, J=11.4, 2.8 Hz, 1H), 3.49 (td, J=11.8, 2.8 Hz, 1H), 3.16 (d, J=3.5 Hz, 1H), 1.54 (s, 6H), 1.19 (d, J=6.7 Hz, 3H).

### Example 17

### (R)-4-(4-Ethyl-1-(1H-pyrazol-3-yl)-1H-pyrazole-[3,4-b]pyridin-6-yl)-3-methylmorpholine

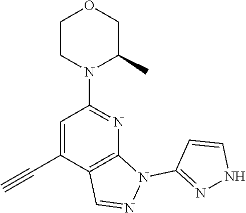

### Step 1: (R)-3-Methyl-4-(1-((2-(trimethylsilyl)ethoxy)methyl)-1H-pyrazol-3-yl)-4-((trimethylsilyl)ethynyl)-1H-pyrazolo[3,4-b]pyridin-6-yl)morpholine

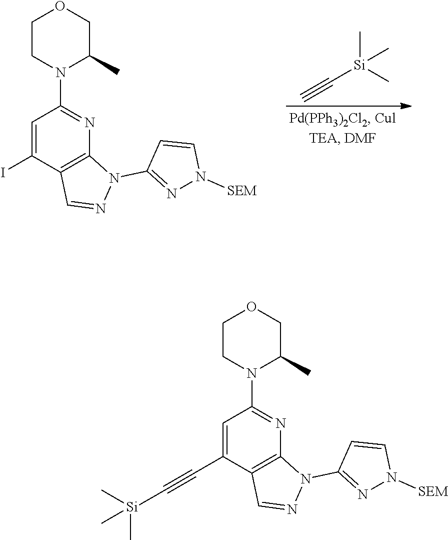

(R)-4-(4-Iodo-1-(1-((2-(trimethylsilyl)ethoxy)methyl)-1H-pyrazol-3-yl)-1H-pyrazolo[3,4-b]pyridin-6-yl)-3-methylmorpholine (120 mg, 0.22 mmol, 1 equiv.), Pd(PPh₃)₂Cl₂ (31 mg, 0.04 mmol, 0.2 equiv.), CuI (8.4 mg, 0.04 mmol, 0.2 equiv.) and triethylamine (44.5 mg, 0.44 mmol, 2 equiv.) were dissolved in DMF (4 mL), and the mixture was stirred at 40° C. for 1 hour under nitrogen protection. After a product was generated as monitored by LCMS, the reaction liquid was cooled to 25° C. The reaction was quenched with water (20 mL), and then the reaction liquid was extracted with ethyl acetate (30 mL). The aqueous phase was washed with ethyl acetate (3*30 mL), and the organic phases were combined, washed with saturated sodium chloride aqueous solution (3*20 mL), dried over anhydrous sodium sulfate, filtered and concentrated, to afford the crude product. The crude product was separated and purified by flash chromatography (100-200 mesh silica gel, petroleum ether:ethyl acetate=0-10%), to afford the title compound (100 mg, yield: 89.5%). LCMS: [M+H]⁺=511.40.

### Step 2: (R)-4-(4-Ethyl-1-(1-((2-(trimethylsilyl)ethoxy)methyl)-1H-pyrazol-3-yl)-1H-pyrazole[3,4-b]pyridin-6-yl)-3-methylmorpholine

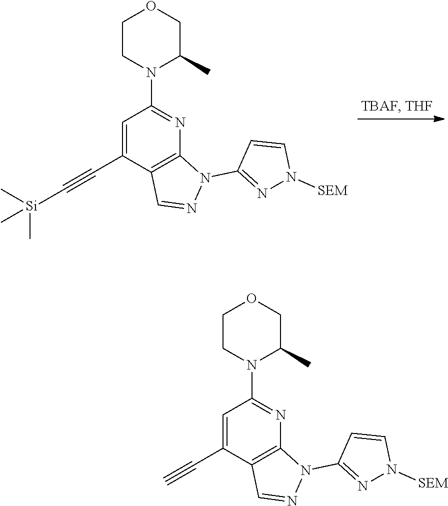 [SMILES: C[C@@H]1COCCN1c1cc(C#C[Si](C)(C)C)c2cnn(-c3ccn(COCC[Si](C)(C)C)n3)c2n1 ; C#Cc1cc(N2CCOC[C@H]2C)nc2c1cnn2-c1ccn(COCC[Si](C)(C)C)n1]

(R)-3-Methyl-4-(1-((2-(trimethylsilyl)ethoxy)methyl)-1H-pyrazol-3-yl)-4-((trimethylsilyl)ethynyl)-1H-pyrazolo[3,4-b]pyridin-6-yl)morpholine (100 mg, 0.19 mmol, 1 equiv.) was dissolved in THF (5 mL), TBAF (1 mL, 2.5 N) was added, and the mixture was stirred at 25° C. for 1 hour. After the raw materials were reacted completely as monitored by TLC, the reaction was quenched with water (20 mL), and then the reaction liquid was extracted with ethyl acetate (30 mL). The aqueous phase was washed with ethyl acetate (3*30 mL), and the organic phases were combined, washed with saturated sodium chloride aqueous solution (3*20 mL), dried over anhydrous sodium sulfate, filtered and concentrated, to afford the crude product (140 mg).

### Step 3: (R)-4-(4-Ethyl-1-(1H-pyrazol-3-yl)-1H-pyrazole-[3,4-b]pyridin-6-yl)-3-methylmorpholine

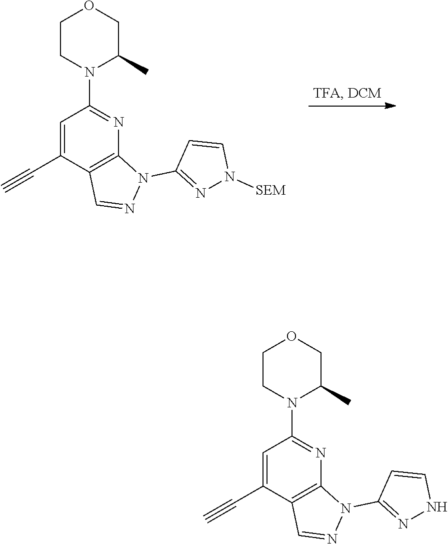 [SMILES: C#Cc1cc(N2CCOC[C@H]2C)nc2c1cnn2-c1ccn(COCC[Si](C)(C)C)n1 ; C#Cc1cc(N2CCOC[C@H]2C)nc2c1cnn2-c1cc[nH]n1]

(R)-4-(4-Ethyl-1-(1-((2-(trimethylsilyl)ethoxy)methyl)-1H-pyrazol-3-yl)-1H-pyrazole[3,4-b]pyridin-6-yl)-3-methylmorpholine (140 mg, 0.32 mmol, 1 equiv.) was dissolved in DCM (5 mL), then TFA (0.5 mL) was added, and the mixture was stirred at 25° C. for 2 h. After the reaction was completed as monitored by LCMS, the reaction was quenched with NaHCO₃ aqueous solution (10 mL), the reaction liquid was extracted with DCM (3*30 mL), and the organic phases were combined, washed with saturated sodium chloride aqueous solution (3*30 mL), dried over anhydrous sodium sulfate, filtered and concentrated, to afford the crude product. The crude product was separated and purified by flash chromatography (C18 reverse-phase column, water:acetonitrile=0-40%), to afford the target compound (20 mg, yield: 34.2%). LCMS: [M+H]⁺=309.22; ¹H NMR: (400 MHz, DMSO-d₆) δ 12.85 (s, 1H), 8.05 (s, 1H), 7.84 (s, 1H), 6.97 (s, 1H), 6.77 (d, J=2.3 Hz, 1H), 4.82 (s, 1H), 4.52-4.42 (m, 1H), 4.10-3.93 (m, 2H), 3.78-3.61 (m, 2H), 3.49 (dd, J=13.2, 10.4 Hz, 1H), 3.20-3.11 (m, 1H), 1.19 (d, J=6.7 Hz, 3H).

### Example 18

### (R)-N, N-Dimethyl-3-(6-(3-methylmorpholine)-1-(1H-pyrazol-3-yl)-1H-pyrazoline[3,4-b]pyridin-4-yl)propyl-2-yn-1-amine

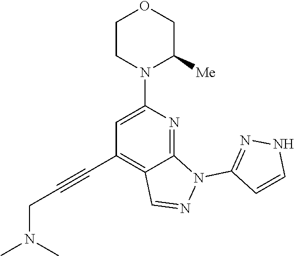

### Step 1: (R)-N, N-Dimethyl-3-(6-(3-methylmorpholine)-1-(1-((2-(trimethylsilyl)ethoxy)methyl)-1H-pyrazol-3-yl)-1H-pyrazole[3,4-b]pyridin-4-yl)propyl-2-yn-1-amine

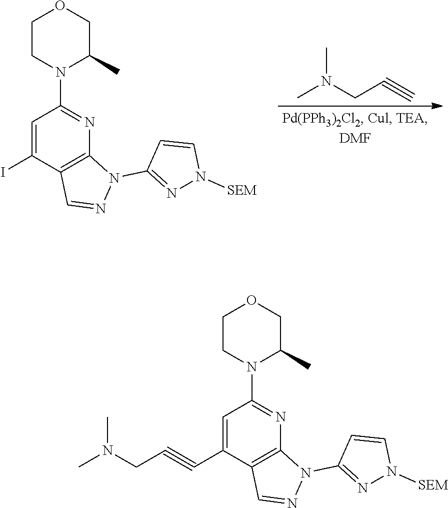

(R)-4-(4-Iodo-1-(1-((2-(trimethylsilyl)ethoxy)methyl)-1H-pyrazol-3-yl)-1H-pyrazolo[3,4-b]pyridin-6-yl)-3-methylmorpholine (120 mg, 0.22 mmol, 1 equiv.), Pd(PPh₃)₂Cl₂ (31 mg, 0.04 mmol, 0.2 equiv.), CuI (8.4 mg, 0.04 mmol, 0.2 equiv.) and triethylamine (44.5 mg, 0.44 mmol, 2 equiv.) were dissolved in DMF (4 mL), and the mixture was stirred at 40° C. for 1 hour under nitrogen protection. After a product was generated as monitored by LCMS, the reaction liquid was cooled to 25° C. The reaction was quenched with water (20 mL), and then the reaction liquid was extracted with ethyl acetate (30 mL). The aqueous phase was washed with ethyl acetate (3*30 mL), and the organic phases were combined, washed with saturated sodium chloride aqueous solution (3*20 mL), dried over anhydrous sodium sulfate, filtered and concentrated, to afford the crude product. The crude product was separated and purified by flash chromatography (100-200 mesh silica gel, petroleum ether:ethyl acetate=0-10%), to afford the target compound (100 mg, yield: 91.8%). For TLC, petroleum ether:ethyl acetate=10:1. The product Rf value was (0.2). LCMS: [M+H]⁺=496.42.

### Step 2: (R)-N, N-Dimethyl-3-(6-(3-methylmorpholine)-1-(1H-pyrazol-3-yl)-1H-pyrazoline[3,4-b]pyridin-4-yl)propyl-2-yn-1-amine

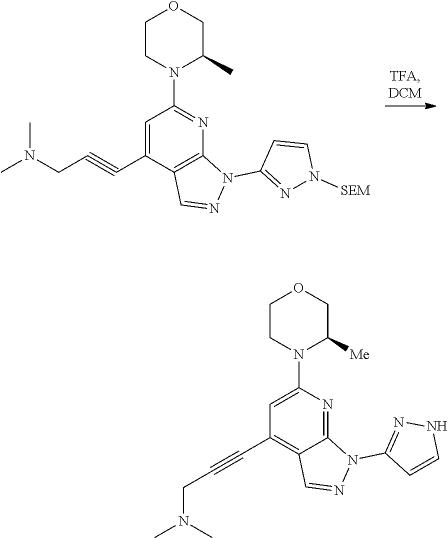 [SMILES: C[C@@H]1COCCN1c1cc(C#CCN(C)C)c2cnn(-c3ccn(COCC[Si](C)(C)C)n3)c2n1 ; C[C@@H]1COCCN1c1cc(C#CCN(C)C)c2cnn(-c3cc[nH]n3)c2n1]

(R)-N, N-Dimethyl-3-(6-(3-methylmorpholine)-1-(1-((2-(trimethylsilyl)ethoxy)methyl)-1H-pyrazol-3-yl)-1H-pyrazole[3,4-b]pyridin-4-yl)propyl-2-yn-1-amine (100 mg, 0.20 mmol, 1 equiv.) was dissolved in DCM (5 mL), then TFA (0.5 mL) was added, and the mixture was stirred at 25° C. for 2 h. After the reaction was completed as monitored by LCMS, the reaction was quenched with NaHCO₃ aqueous solution (10 mL), the reaction liquid was extracted with DCM (3*30 mL), and the organic phases were combined, washed with saturated sodium chloride aqueous solution (3*30 mL), dried over anhydrous sodium sulfate, filtered and concentrated, to afford the crude product. The crude product was separated and purified by flash chromatography (C18 reverse-phase column, water:acetonitrile=0-50%), to afford the title compound (38 mg, yield: 52.0%). For TLC, petroleum ether:ethyl acetate=1:2. The product Rf value was (0.2). LCMS: [M+H]⁺=366.1; ¹H NMR: (400 MHz, DMSO-d6) δ 12.85 (s, 1H), 8.01 (s, 1H), 7.84 (s, 1H), 6.91 (s, 1H), 6.77 (s, 1H), 4.47 (d, J=6.2 Hz, 1H), 4.06 (d, J=12.8 Hz, 1H), 3.96 (dd, J=11.3 Hz, 3.3 Hz, 1H), 3.75 (d, J=11.4 Hz, 1H), 3.68-3.60 (m, 3H), 3.49 (td, J=11.9, 2.9 Hz, 1H), 3.17 (td, J=12.9, 3.7 Hz, 1H), 2.33 (s, 6H), 1.19 (d, J=6.7 Hz, 3H).

### Examples 19-29

The following compounds of Examples 19-29 were prepared with reference to the preparation methods of Examples 1-18.

| Example | | |
|---|---|---|
| No. | Structure | LCMS (ESI) [M + H]⁺ |
| 19 | 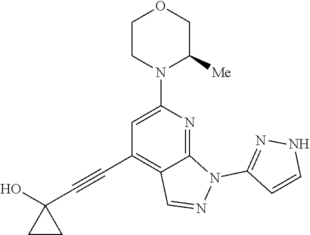 | 365.16 |
| 20 | 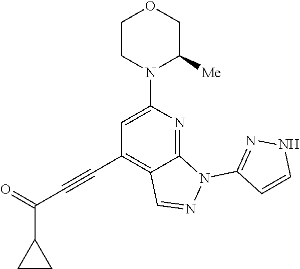 | 377.16 |
| 21 | 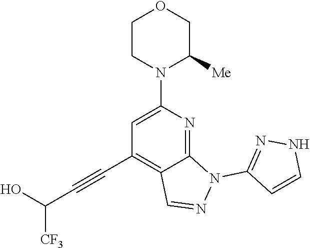 | 407.14 |
| 22 | 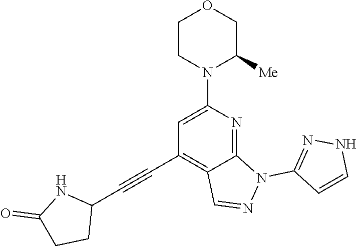 | 392.18 |
| 23 | 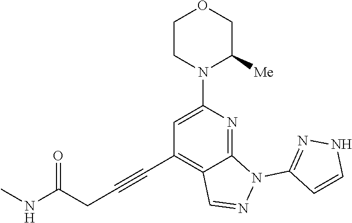 | 380.18 |
| 24 | 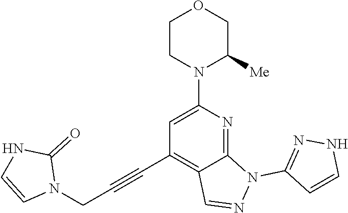 | 405.18 |
| 25 | 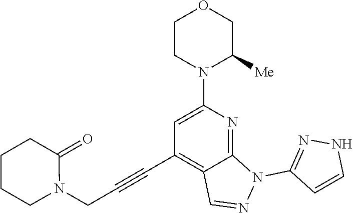 | 420.21 |
| 26 | 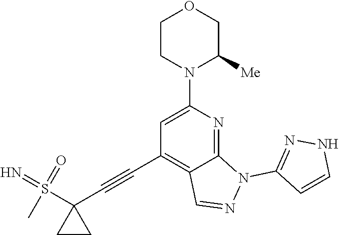 | 426.16 |
| 27 | 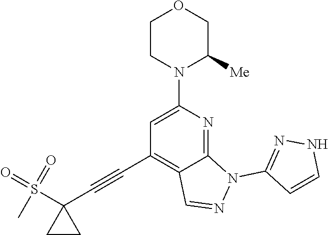 | 427.15 |
| 28 | 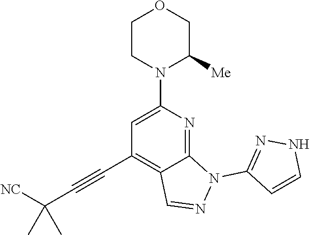 | 376.18 |
| 29 | 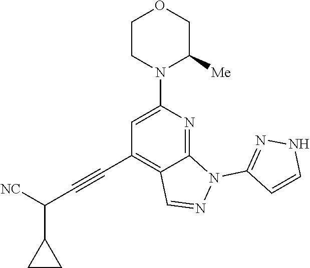 | 388.18 |

### Example 30

### Preparation of (R)-N-(4-(6-(3-methylmorpholinyl)-1-(1H-pyrazolyl-3-yl)-1H-pyrazolyl[3,4-b]pyridin-4-yl)phenyl)acetamide

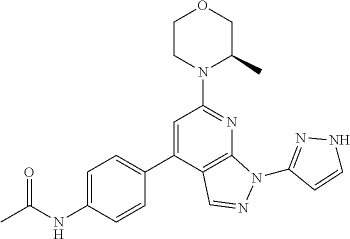 [SMILES: CC(=O)Nc1ccc(-c2cc(N3CCOC[C@H]3C)nc3c2cnn3-c2cc[nH]n2)cc1]

### Step 1: Preparation of (R)-N-(4-(6-(3-methylmorpholinyl)-1-(1-(2-(trimethylsilyl)ethoxy)methyl)-1H-pyrazol-3-yl)-1H-pyrazole[3,4-b]pyridin-4-yl)phenyl)acetamide

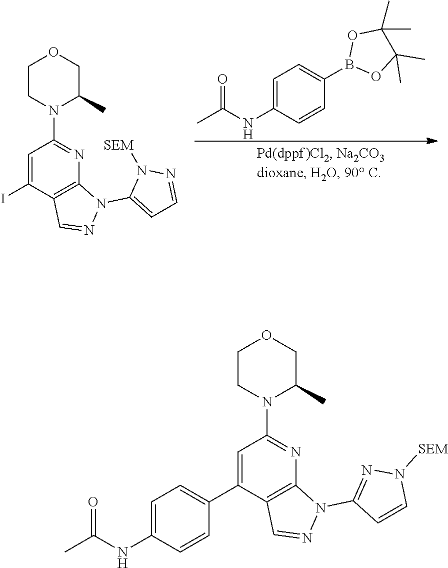 [SMILES: C[C@@H]1COCCN1c1cc(I)c2cnn(-c3ccnn3COCC[Si](C)(C)C)c2n1 ; CC(=O)Nc1ccc(B2OC(C)(C)C(C)(C)O2)cc1 ; CC(=O)Nc1ccc(-c2cc(N3CCOC[C@H]3C)nc3c2cnn3-c2ccn(COCC[Si](C)(C)C)n2)cc1]

(R)-4-(4-Iodo-1-(1-((2-(trimethylsilyl)ethoxy)methyl)-1H-pyrazol-5-yl)-1H-pyrazole[3,4-b]pyridin-6-yl)-3-methylmorpholine (50.0 mg, 0.09 mmol, 1.0 equiv.), N-(4,4,5,5-tetramethyl-1,3,2-dioxaborolan-2-yl)phenyl)acetamide (18.0 mg, 0.10 mmol, 1.1 equiv.), [1,1′-bis(diphenylphosphino)ferrocene]dichloropalladium (II) (7.3 mg, 0.01 mmol, 0.1 equiv.) and sodium carbonate (28.6 mg, 0.27 mmol, 3.0 equiv.) were weighed and dissolved in 1,4-dioxane (4 mL) and water (2 mL), and the mixture was heated to 90° C. and reacted at this temperature for 3 hours after nitrogen replacement was performed three times. After the raw materials were reacted completely as monitored by TLC and a product was generated as monitored by LCMS, the reaction liquid was filtered and extracted with ethyl acetate (20 mL×3), the organic phases were combined and spun to dryness, and the residue was purified by thin layer chromatography (petroleum ether:ethyl acetate=2:1), to afford the target compound (30.0 mg, yield: 60.8%). LCMS (ESI) [M+H]⁺=548.55.

### Step 2: Preparation of (R)-N-(4-(6-(3-methylmorpholinyl)-1-(1H-pyrazolyl-3-yl)-1H-pyrazolyl[3,4-b]pyridin-4-yl)phenyl)acetamide

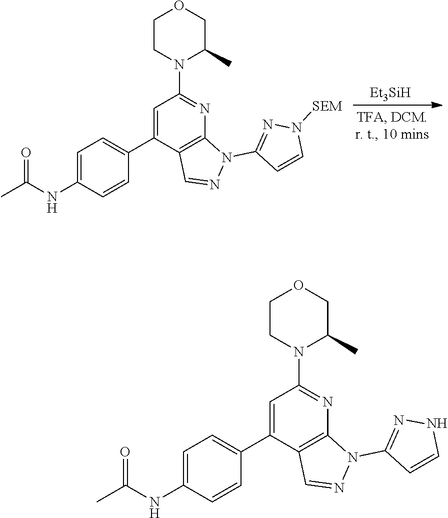 [SMILES: "Et3SiH" ; CC(=O)Nc1ccc(-c2cc(N3CCOC[C@H]3C)nc3c2cnn3-c2ccn(COCC[Si](C)(C)C)n2)cc1 ; CC(=O)Nc1ccc(-c2cc(N3CCOC[C@H]3C)nc3c2cnn3-c2cc[nH]n2)cc1]

(R)-N-(4-(6-(3-Methylmorpholinyl)-1-(1-(2-(trimethylsilyl)ethoxy)methyl)-1H-pyrazol-3-yl)-1H-pyrazole[3,4-b]pyridin-4-yl)phenyl)acetamide (30.0 mg, 0.05 mmol, 1.0 equiv.) was dissolved in dichloromethane (2 mL), trifluoroacetic acid (2 mL) and triethylsilane (0.2 mL) were added, and the mixture was reacted at 25° C. for 10 minutes after nitrogen replacement was performed three times. After the raw materials were reacted completely as monitored by TLC and a product was generated as monitored by LCMS, the reaction liquid was adjusted to be alkaline (pH) with saturated sodium bicarbonate and extracted with ethyl acetate (20 mL×3), the organic phases were combined and spun to dryness, and the residue was purified by thin layer chromatography (dichloromethane:methanol=10:1), to afford the target compound (8.8 mg, yield: 39.1%). LCMS (ESI) [M+H]⁺=418.20; ¹H NMR (400 MHz, DMSO-d6) δ 12.80 (s, 1H), 10.16 (s, 1H), 8.16 (s, 1H), 7.78 (dd, J=19.0, 8.4 Hz, 5H), 6.83 (d, J=26.3 Hz, 2H), 4.53 (s, 1H), 4.11 (d, J=13.3 Hz, 1H), 3.95 (d, J=10.9 Hz, 1H), 3.75 (d, J=11.2 Hz, 1H), 3.64 (d, J=11.6 Hz, 1H), 3.49 (t, J=10.9 Hz, 1H), 3.19 (d, J=11.8 Hz, 1H), 2.07 (s, 3H), 1.19 (d, J=6.4 Hz, 3H).

### Example 31

### Preparation of (R)-N-(4-(6-(3-methylmorpholino)-1-(1H-pyrazol-3-yl)-1H-pyrazolo[3,4-b]pyridin-4-yl)phenyl)-2-oxopropanamide

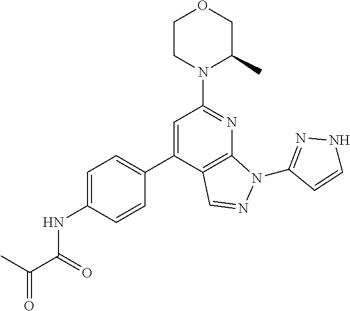 [SMILES: CC(=O)C(=O)Nc1ccc(-c2cc(N3CCOC[C@H]3C)nc3c2cnn3-c2cc[nH]n2)cc1]

### Step 1: Preparation of 2-oxo-N-(4-(4,4,5,5-tetramethyl-1,3,2-dioxaborolan-2-yl)phenyl)propanamide

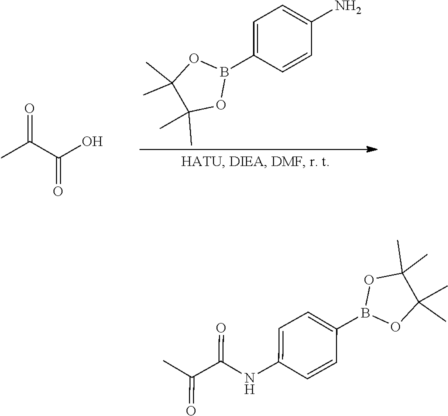

2-Oxopropanoic acid (200 mg, 2.27 mmol, 1.1 equiv.) was dissolved in N,N-dimethylformamide (2 mL), 2-(7-azabenzotriazole)-N,N,N′,N′-tetramethyluronium hexafluorophosphate (1.02 g, 2.68 mmol, 1.3 equiv.) was added under ice bath, and the mixture was stirred at room temperature for 30 minutes. 4-(4,4,5,5-Tetramethyl-1,3,2-dioxaborolan-2-yl)aniline (452.4 mg, 2.1 mmol, 1.0 equiv.) and N,N-diisopropylethylamine (800 mg, 6.2 mmol, 3.0 equiv.) dissolved in N,N-dimethylformamide (2 mL) were added, and the mixture was stirred at room temperature for 2 hours. After the raw materials were reacted completely as monitored by TLC, the reaction was quenched with water (20 mL), and the reaction liquid was extracted with ethyl acetate (30 mL). The aqueous phase was washed with ethyl acetate (3×30 mL), and the organic phases were combined, washed with saturated sodium chloride aqueous solution (3×20 mL), dried over anhydrous sodium sulfate, filtered and concentrated, to afford the crude product. The crude product was purified by column chromatography (petroleum ether:ethyl acetate=20:1), to afford the target compound (460 mg, yield: 70.11%). LCMS (ESI) [M+H]⁺=290.16.

### Step 2: Preparation of (R)-N-(4-(6-(3-methylmorpholino)-1-(1-((2-(trimethylsilyl)ethoxy)methyl)-1H-pyrazol-3-yl)-1H-pyrazolo[3, 4-b]pyridin-4-yl)phenyl)-2-oxopropanamide

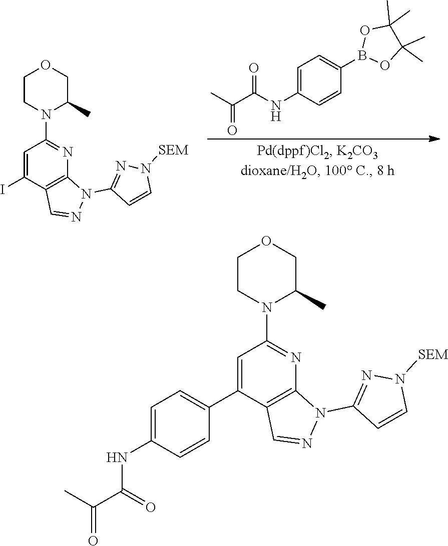

(R)-4-(4-Iodo-1-(1-((2-(trimethylsilyl)ethoxy)methyl)-1H-pyrazol-3-yl)-1H-pyrazolo[3,4-b]pyridin-6-yl)-3-methylmorpholine (100 mg, 0.18 mmol, 1.0 equiv.), 2-oxo-N-(4-(4,4,5,5-tetramethyl-1,3,2-dioxaborolan-2-yl)phenyl)propanamide (62.4 mg, 0.21 mmol, 1.2 equiv.), Pd(dppf)Cl₂ (26 mg, 0.04 mmol, 0.2 equiv.) and potassium carbonate (50 mg. 0.36 mmol, 2.0 equiv.) were dissolved in 1,4-dioxane (3 mL) and water (0.3 mL), and the mixture was reacted at 100° C. under nitrogen protection for 8 hours. After the reaction was completed as monitored by TLC, the reaction liquid was cooled to room temperature. The solid was filtered, and the filtrate was spun to dryness, to afford the crude product. The crude product was purified by column chromatography (petroleum ether:ethyl acetate=2:1), to afford the target compound (88 mg, yield: 85.02%). LCMS (ESI) [M+H]⁺=576.42.

### Step 3: Preparation of (R)-N-(4-(6-(3-methylmorpholino)-1-(1H-pyrazol-3-yl)-1H-pyrazolo[3,4-b]pyridin-4-yl)phenyl)-2-oxopropanamide

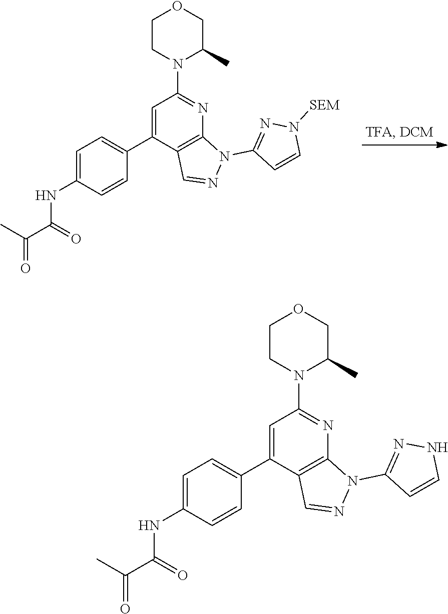

(R)-N-(4-(6-(3-Methylmorpholino)-1-(1-((2-(trimethylsilyl)ethoxy)methyl)-1H-pyrazol-3-yl)-1H-pyrazolo[3, 4-b]pyridin-4-yl)phenyl)-2-oxopropanamide (88 mg) was dissolved in dichloromethane (5 mL), trifluoroacetic acid (2 mL) was added, and the mixture was stirred at room temperature for 4 hours. After the reaction was completed as monitored by LCMS, the reaction liquid was spun to dryness to remove the solvent. The reaction was quenched with sodium bicarbonate aqueous solution (10 mL), the reaction liquid was extracted with ethyl acetate (3×30 mL), and the organic phases were combined, washed with saturated sodium chloride aqueous solution (3×30 mL), dried over anhydrous sodium sulfate, filtered and concentrated, to afford the crude product. The crude product was purified by reverse-phase column chromatography (C18, water:acetonitrile=1:1), to afford the target compound (15 mg, yield: 18.73%). LCMS (ESI) [M+H]⁺=446.34; ¹H NMR (400 MHz, DMSO-d6) δ12.85 (s, 1H), 10.69 (s, 1H), 8.21 (s, 1H), 8.05 (d, J=8.6 Hz, 2H), 7.89 (d, J=8.6 Hz, 2H), 7.85 (s, 1H), 6.93 (s, 1H), 6.83 (d, J=1.9 Hz, 1H), 4.65-4.51 (m, 1H), 4.23-4.09 (m, 1H), 3.99 (dd, J=11.1, 2.9 Hz, 1H), 3.78 (d, J=11.1 Hz, 1H), 3.68 (d, J=11.3 Hz, 1H), 3.52 (t, J=11.8 Hz, 1H), 3.25-3.17 (m, 2H), 2.47 (s, 3H), 1.23 (d, J=6.8 Hz, 4H).

### Example 32

### Preparation of (R)-N-(4-(6-(3-methylmorpholinyl)-1-(1H-pyrazolyl-3-yl)-1H-pyrazolyl[3,4-b]pyridin-4-yl)phenyl)cyclobutanecarboxamide

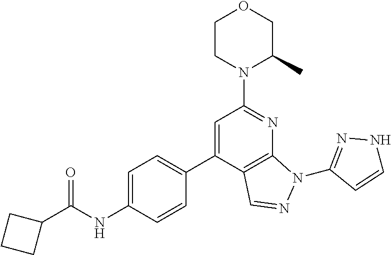 [SMILES: C[C@@H]1COCCN1c1cc(-c2ccc(NC(=O)C3CCC3)cc2)c2cnn(-c3cc[nH]n3)c2n1]

### Step 1: Preparation of N-(4-(4,4,5,5-tetramethyl-1,3,2-dioxaborolan-2-yl)phenyl)cyclobutaneamide

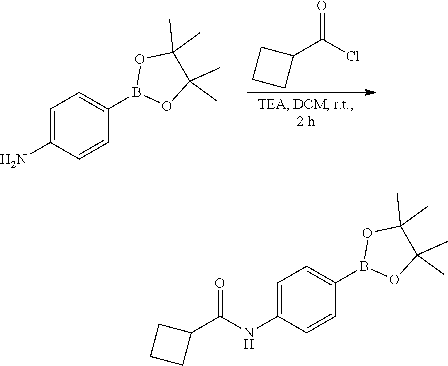

p-Aminophenylborate (300.0 mg, 1.37 mmol, 1.0 equiv.) and triethylamine (415.0 mg, 4.11 mmol, 3.0 equiv) were weighed and dissolved in dichloromethane (5 mL), cyclobutanecarbonyl chloride (195.0 mg, 1.64 mmol, 1.2 equiv) was added at 0° C., and the mixture was reacted at 25° C. for 2 hours. After the reaction was completed as monitored by TLC, the reaction liquid was subjected to liquid separation by adding water (200 mL) and ethyl acetate (80 mL×3), the organic phases were combined and spun to dryness, and the residue was purified by thin layer chromatography (ethyl acetate:petroleum ether=1:3), to afford the target compound (200.0 mg, yield: 48.5%). LCMS (ESI) [M+H]⁺=302.15.

### Step 2: Preparation of (R)-N-(4-(6-(3-methylmorpholinyl)-1-(1-(2-(trimethylsilyl)ethoxy)methyl)-1H-pyrazolyl-3-yl)-1H-pyrazolyl[3,4-b]pyridin-4-yl)phenyl)cyclobutanecarboxamide

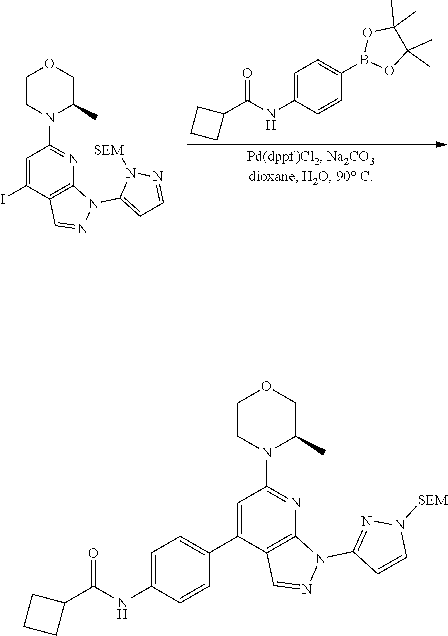

(R)-4-(4-Iodo-1-(1-((2-(trimethylsilyl)ethoxy)methyl)-1H-pyrazol-5-yl)-1H-pyrazole[3,4-b]pyridin-6-yl)-3-methylmorpholine (50.0 mg, 0.09 mmol, 1.0 equiv), N-(4-(4,4,5,5-tetramethyl-1,3,2-dioxaborolan-2-yl)phenyl)cyclobutaneamide (37.0 mg, 0.10 mmol, 1.1 equiv), [1,1′-bis(diphenylphosphino)ferrocene]dichloropalladium (II) (7.0 mg, 0.01 mmol, 0.1 equiv) and sodium carbonate (20.0 mg, 0.18 mmol, 2.0 equiv) were dissolved in 1,4-dioxane (2 mL) and water (1 mL), and the mixture was heated to 90° C. and stirred for 2 hours. After the reaction was completed as monitored by LCMS, the reaction liquid was filtered and subjected to liquid separation by adding water (50 mL) and ethyl acetate (20 mL×3), the organic phase was dried, filtered and concentrated, and the residue was purified by thin layer chromatography (ethyl acetate:petroleum ether=1:1), to afford the target compound (47.0 mg, yield: 87.0%). LCMS (ESI) [M+H]⁺=588.35.

### Step 3: Preparation of (R)-N-(4-(6-(3-methylmorpholinyl)-1-(1H-pyrazolyl-3-yl)-1H-pyrazolyl[3,4-b]pyridin-4-yl)phenyl)cyclobutanecarboxamide

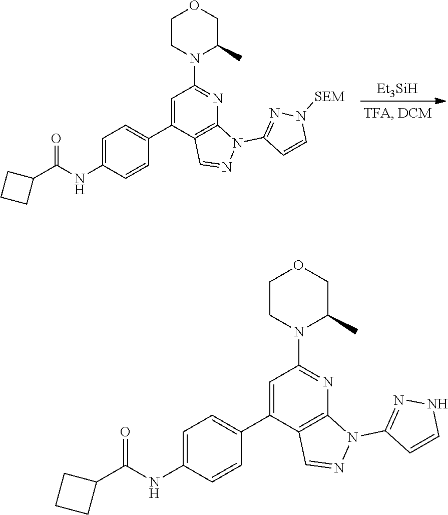

(R)-N-(4-(6-(3-Methylmorpholinyl)-1-(1-(2-(trimethylsilyl)ethoxy)methyl)-1H-pyrazolyl-3-yl)-1H-pyrazolyl[3,4-b]pyridin-4-yl)phenyl)cyclobutanecarboxamide (47.0 mg, 0.08 mmol, 1.0 equiv) was dissolved in dichloromethane (1 mL), trifluoroacetic acid (1 mL) and triethylsilane (0.1 mL) were added, and the mixture was reacted at room temperature for 1 hour. After the raw materials were reacted completely as monitored by TLC, the reaction was quenched with water (50 mL), and the reaction liquid was extracted with dichloromethane (20 mL×3). The organic phases were combined, dried and spun to dryness, and the residue was purified by thin layer chromatography (dichloromethane:methanol=10:1), to afford the target compound (19.0 mg, yield: 52.7%). LCMS (ESI) [M+H]⁺=458.25; ¹H NMR (400 MHz, DMSO-d6) δ 12.80 (s, 1H), 9.93 (s, 1H), 8.16 (s, 1H), 7.81 (d, J=12.8 Hz, 5H), 6.83 (d, J=24.9 Hz, 2H), 4.54 (s, 1H), 4.11 (d, J=12.6 Hz, 1H), 3.96 (d, J=10.0 Hz, 1H), 3.75 (d, J=11.2 Hz, 1H), 3.64 (d, J=11.7 Hz, 1H), 3.49 (q, J=11.0 Hz, 1H), 3.23 (dd, J=21.6, 13.0 Hz, 2H), 2.26-2.18 (m, 2H), 2.11 (d, J=8.2 Hz, 2H), 1.99-1.90 (m, 1H), 1.81 (s, 1H), 1.19 (d, J=6.4 Hz, 3H).

### Example 33

### Preparation of (R)-1-cyclopropyl-3-(4-(6-(3-methylmorpholinyl)-1-(1H-pyrazolyl-3-yl)-1H-pyrazolyl[3,4-b]pyridin-4-yl)phenyl)urea

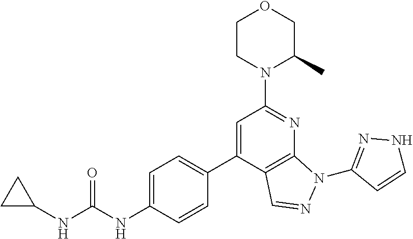 [SMILES: C[C@@H]1COCCN1c1cc(-c2ccc(NC(=O)NC3CC3)cc2)c2cnn(-c3cc[nH]n3)c2n1]

### Step 1: Preparation of (R)-1-cyclopropyl-3-(4-(6-(3-methylmorpholinyl)-1-(1-(2-(trimethylsilyl)ethoxy)methyl)-1H-pyrazol-3-yl)-1H-pyrazole[3,4-b]pyridin-4-yl)phenyl)urea

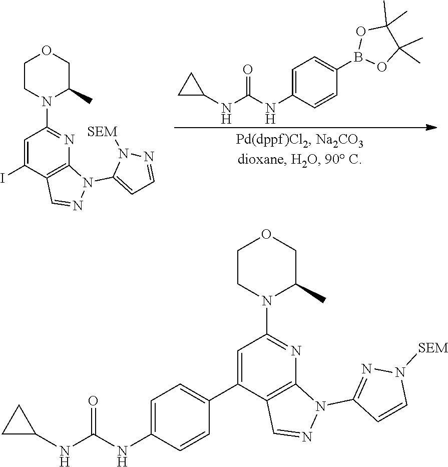

(R)-4-(4-Iodo-1-(1-((2-(trimethylsilyl)ethoxy)methyl)-1H-pyrazol-5-yl)-1H-pyrazole[3,4-b]pyridin-6-yl)-3-methylmorpholine (50.0 mg, 0.09 mmol, 1.0 equiv.), 1-cyclopropyl-3-(4-(4,4,5,5-tetramethyl-1,3,2-dioxaborolan-2-yl)phenyl)urea (30.0 mg, 0.10 mmol, 1.1 equiv.), [1,1′-bis(diphenylphosphino)ferrocene]dichloropalladium (II) (7.3 mg, 0.01 mmol, 0.1 equiv) and sodium carbonate (28.6 mg, 0.27 mmol, 3.0 equiv.) were weighed and dissolved in 1,4-dioxane (4 mL) and water (2 mL), and the mixture was heated to 90° C. and reacted at this temperature for 3 hours after nitrogen replacement was performed three times. After the raw materials were reacted completely as monitored by TLC and a product was generated as monitored by LCMS, the reaction liquid was filtered and extracted with ethyl acetate (20 mL×3), the organic phases were combined and spun to dryness, and the residue was purified by thin layer chromatography (petroleum ether:ethyl acetate=2:1), to afford the target compound (40.0 mg, yield: 74.1%). LCMS (ESI) [M+H]⁺=589.10.

### Step 2: Preparation of (R)-1-cyclopropyl-3-(4-(6-(3-methylmorpholinyl)-1-(1H-pyrazolyl-3-yl)-1H-pyrazolyl[3,4-b]pyridin-4-yl)phenyl)urea

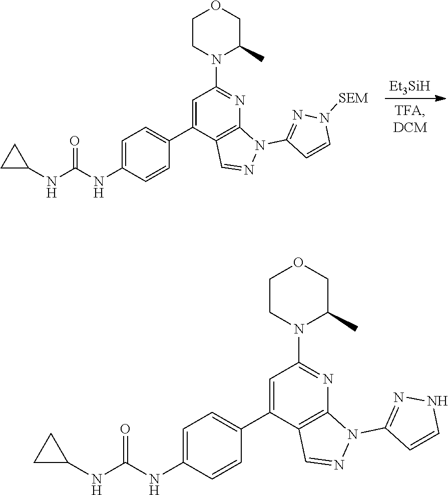

(R)-1-Cyclopropyl-3-(4-(6-(3-methylmorpholinyl)-1-(1-(2-(trimethylsilyl)ethoxy)methyl)-1H-pyrazol-3-yl)-1H-pyrazole[3,4-b]pyridin-4-yl)phenyl)urea (40.0 mg, 0.07 mmol, 1.0 equiv.) was dissolved in dichloromethane (2.0 mL), trifluoroacetic acid (2.0 mL) and triethylsilane (0.2 mL) were added, and the mixture was reacted at 25° C. for 1 hour after nitrogen replacement was performed three times. After the raw materials were reacted completely as monitored by TLC and a product was generated as monitored by LCMS, the reaction was quenched with saturated sodium bicarbonate (50.0 mL), and the reaction liquid was extracted with ethyl acetate (20 mL×3). The organic phases were combined and spun to dryness, and the residue was purified by thin layer chromatography (dichloromethane:methanol=10:1), to afford the target compound (6.4 mg, yield: 20.5%). LCMS (ESI) [M+H]+=459.20; ¹H NMR (400 MHz, DMSO-d6) δ 12.83 (s, 1H), 8.57 (s, 1H), 8.16 (s, 1H), 7.80 (s, 1H), 7.74 (d, J=8.5 Hz, 2H), 7.58 (d, J=8.6 Hz, 2H), 6.82 (d, J=17.0 Hz, 2H), 6.45 (s, 1H), 4.53 (s, 1H), 4.11 (d, J=13.2 Hz, 1H), 3.96 (d, J=10.4 Hz, 1H), 3.74 (d, J=11.8 Hz, 1H), 3.64 (d, J=9.0 Hz, 1H), 3.49 (s, 1H), 3.18 (s, 1H), 2.54 (s, 1H), 1.20 (s, 3H), 0.62 (d, J=6.8 Hz, 2H), 0.40 (s, 2H).

### Examples 34-58

The following compounds of Examples 34-58 were prepared with reference to the preparation methods of Examples 1-18 and 30-33.

| | | LCMS | |
|---|---|---|---|
| Example | | (ESI) | |
| No. | Structure | [M + H]⁺ | ¹H NMR |
| 34 | 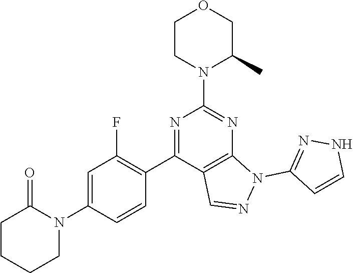 | 477.21 | |
| 35 | 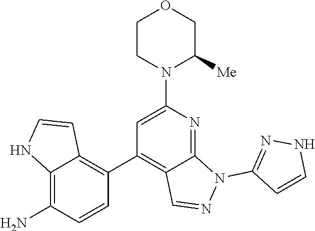 | 415.19 | |
| 36 | 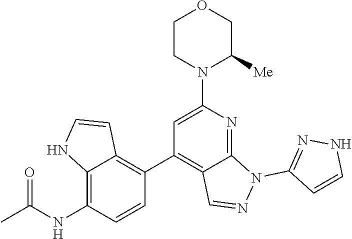 | 457.20 | |
| 37 | 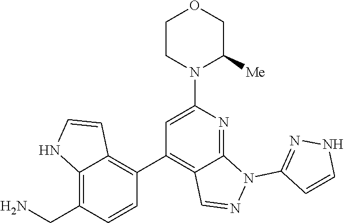 | 429.21 | |
| 38 | 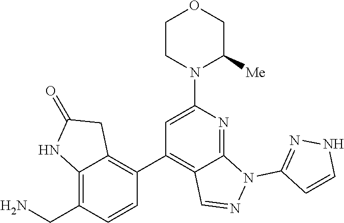 | 445.20 | |
| 39 | 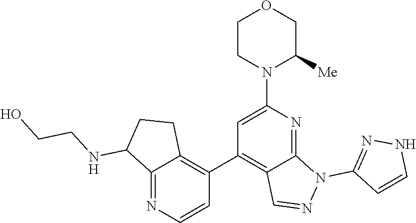 | 461.23 | |
| 40 | 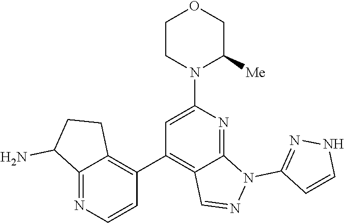 | 417.21 | |
| 41 | 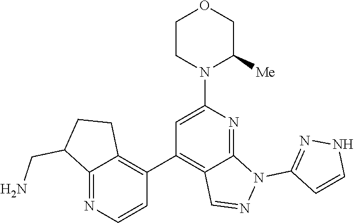 | 431.22 | |
| 42 | 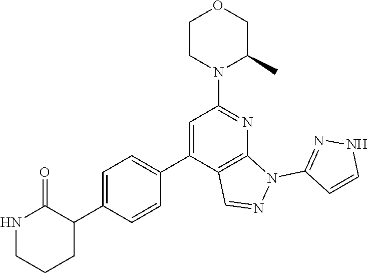 | 458.2 | |
| 43 | 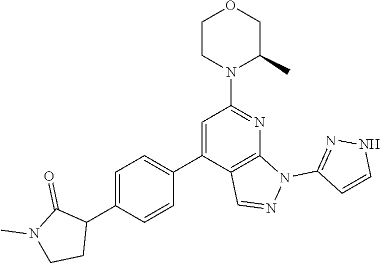 | 458.22 | |
| 44 | 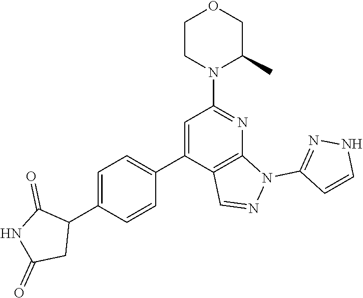 | 458.20 | |
| 45 | 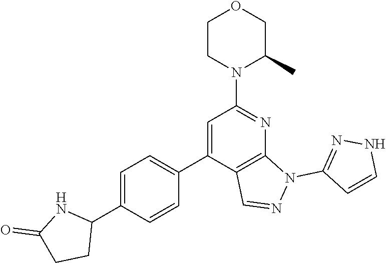 | 444.21 | |
| 46 | 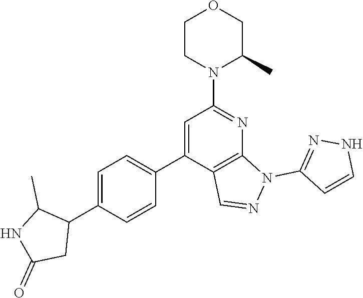 | 458.22 | |
| 47 | 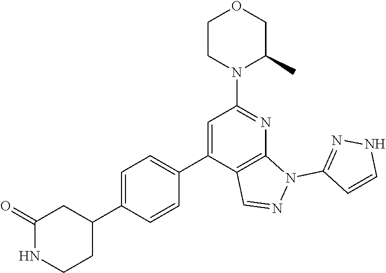 | 458.22 | |
| 48 | 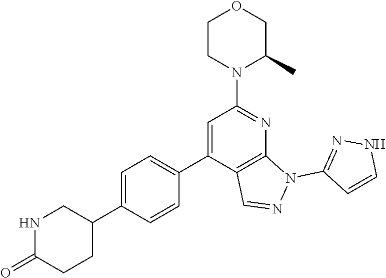 | 458.22 | |
| 49 | 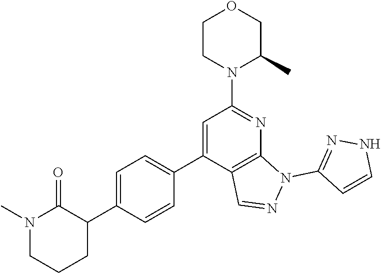 | 472.24 | |
| 50 | 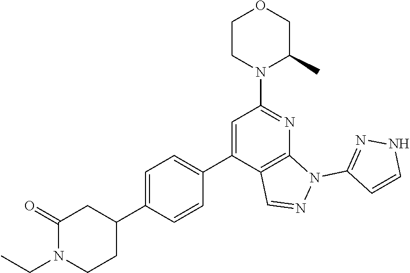 | 486.25 | |
| 51 | 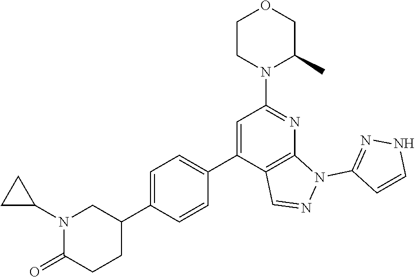 | 498.25 | |
| 52 | 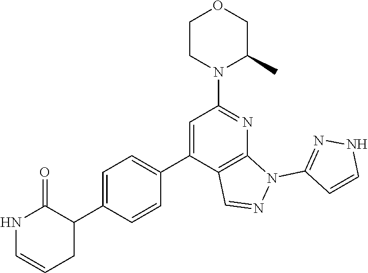 | 456.21 | |
| 53 | 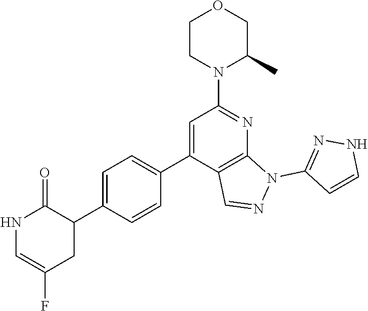 | 474.20 | |
| 54 | 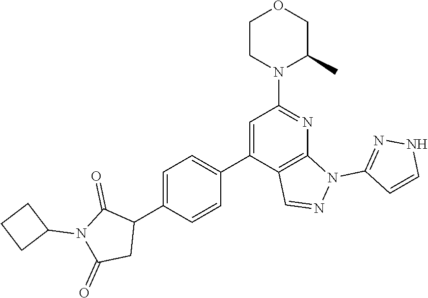 | 512.23 | |
| 55 | 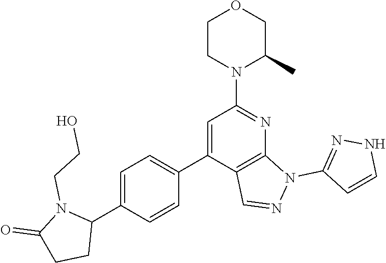 | 488.23 | |
| 56 | 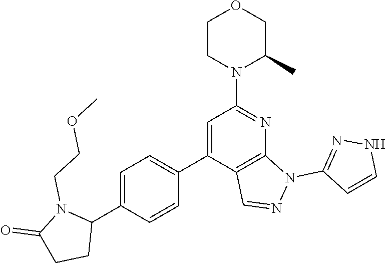 | 502.25 | |
| 57 | 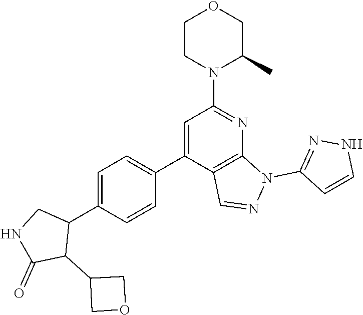 | 500.23 | |
| 58 | 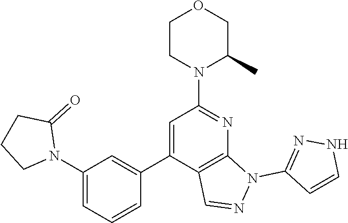 | 444.37 | ¹H NMR (400 MHz, DMSO-d6) δ 12.85 (s, 1H), 8.18 (d, J = 22.2 Hz, 2H), 7.86 (d, J = 1.5 Hz, 1H), 7.81- 7.74 (m, 1H), 7.65-7.54 (m, 2H), 6.94 (s, 1H), 6.83 (d, J = 2.0 Hz, 1H), 4.63- 4.51 (m, 1H), 4.15 (d, J = 13.2 Hz, 1H), 3.97 (dd, J = 14.8, 7.7 Hz, 3H), 3.78 (d, J = 11.3 Hz, 1H), 3.68 (dd, J = 11.4, 2.7 Hz, 1H), 3.57-3.48 (m, 1H), 3.22 (td, J = 13.0, 3.7 Hz, 1H), 2.55 (t, J = 8.0 Hz, 2H), 2.16-2.06 (m, 2H), 1.23 (d, J = 6.6 Hz, 3H) |

### Example 59

### Preparation of (R)-1-(3-fluoro-4-(6-(3-methylmorpholino)-1-(1H-pyrazol-3-yl)-1H-pyrazolo[3,4-b]pyridin-4-yl)phenyl)pyrrolidin-2-one

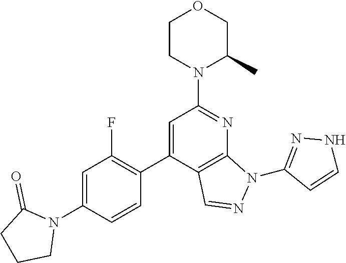 [SMILES: C[C@@H]1COCCN1c1cc(-c2ccc(N3CCCC3=O)cc2F)c2cnn(-c3cc[nH]n3)c2n1]

### Step 1: Preparation of 1-(4-bromo-3-fluorophenyl)pyrrolidin-2-one

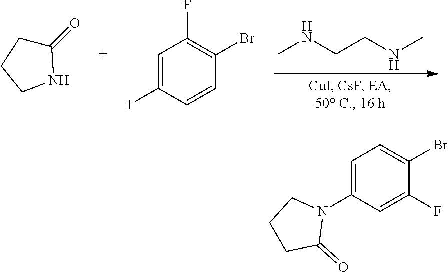

2-Pyrrolidone (1 g, 11.7 mmol, 1 equiv.), 1-bromo-2-fluoro-4-iodobenzene (3.53 g, 11.7 mmol, 1 equiv.), N,N-dimethyl-1,2-ethylenediamine (117 mg, 1.1 mmol, 0.1 equiv.), CsF (3.55 g, 23.4 mmol, 2 equiv.) and CuI (223 mg, 1.1 mmol, 0.1 equiv.) were dissolved in ethyl acetate (30 mL), and the mixture was reacted at 50° C. under nitrogen protection for 16 hours. After LCMS indicated that the product was a major peak, the temperature was lowered to room temperature and the solid was filtered. The filtrate was spun to dryness to afford the crude product, and the crude product was separated and purified by column chromatography (petroleum ether:ethyl acetate=3:1), to afford the target compound (1.7 g, yield: 56.54%). LCMS (ESI) [M+H]⁺=258.05.

### Step 2: Preparation of 1-(3-fluoro-4-(4,4,5,5-tetramethyl-1,3,2-dioxaborolan-2-yl)phenyl)pyrrolidone-2-one

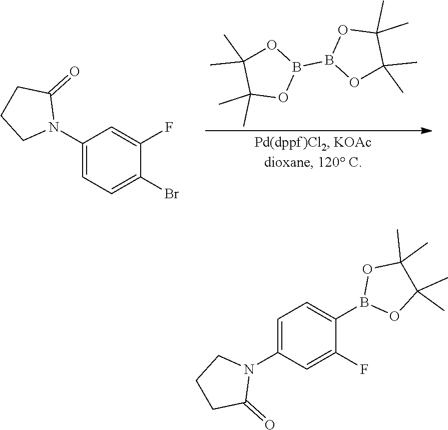

1-(4-Bromo-3-fluorophenyl)pyrrolidin-2-one (700 mg, 2.7 mmol, 1 equiv.), bis(pinacolato)diboron (1.38 g, 5.4 mmol, 2 equiv.), Pd(dppf)Cl₂ (197 mg, 0.27 mmol, 0.1 equiv.) and KOAc (529.2 mg, 5.4 mmol, 2 equiv.) were dissolved in 1, 4-dioxane (30 mL), and the mixture was reacted at 120° C. under nitrogen protection for 8 hours. After LCMS indicated that the product was a major peak, the temperature was lowered to room temperature. The solid was filtered, and the filtrate was spun to dryness, to afford the crude product. The crude product was purified by column chromatography (petroleum ether:ethyl acetate=3:1), to afford the target compound (800 mg, yield: 97.14%). LCMS (ESI) [M+H]⁺=306.23.

### Step 3: Preparation of (R)-1-(3-fluoro-4-(6-(3-methylmorpholine)-1-(2-(2-(trimethylsilyl)ethoxymethyl)-1H-pyrazol-3-yl)-1H-pyrazol-3-yl)-1H-pyrazole[3,4-b]pyridin-4-ylphenyl)-pyrrol-2-one

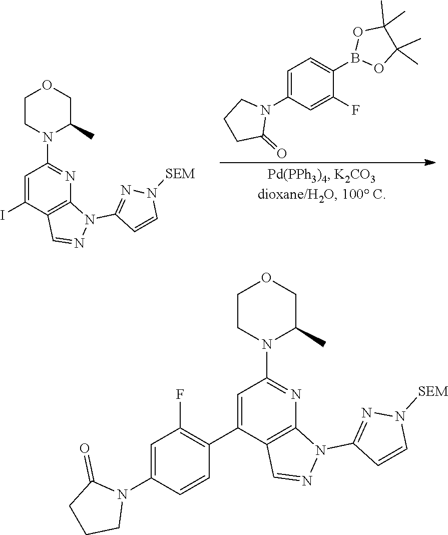

(R)-4-(4-Iodo-1-(1-((2-(trimethylsilyl)ethoxy)methyl)-1H-pyrazol-3-yl)-1H-pyrazolo[3,4-b]pyridin-6-yl)-3-methylmorpholine (100 mg, 0.18 mmol, 1.0 equiv.), 1-(3-fluoro-4-(4,4,5,5-tetramethyl-1,3,2-dioxaborolan-2-yl)phenyl)pyrrolidone-2-one (113 mg, 0.36 mmol, 2.0 equiv.), Pd(PPh₃)₄ (42 mg, 0.04 mmol, 0.2 equiv.) and K₂CO₃ (50 mg. 0.36 mmol, 2 equiv.) were dissolved in 1, 4-dioxane (5 mL) and water (0.5 mL), and the mixture was reacted at 100° C. under nitrogen protection for 4 hours. After the reaction was completed as monitored by LCMS, the reaction liquid was cooled to room temperature. The solid was filtered, and the filtrate was spun to dryness, to afford the crude product. The crude product was purified by column chromatography (petroleum ether:ethyl acetate=1:1), to afford the target compound (100 mg, yield: 93.93%). LCMS (ESI) [M+H]⁺=592.43.

### Step 4: Preparation of (R)-1-(3-fluoro-4-(6-(3-methylmorpholino)-1-(1H-pyrazol-3-yl)-1H-pyrazolo[3,4-b]pyridin-4-yl)phenyl)pyrrolidin-2-one

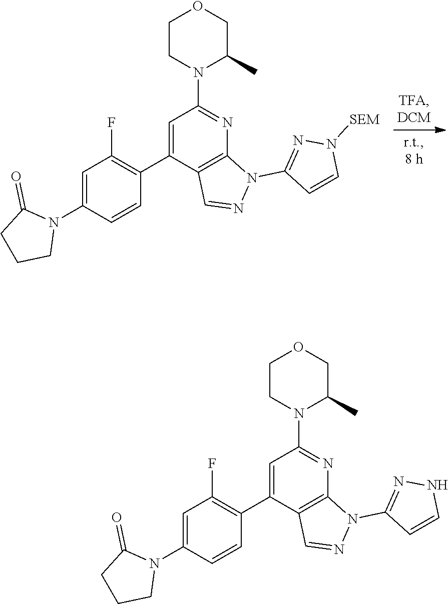

(R)-1-(3-Fluoro-4-(6-(3-methylmorpholine)-1-(2-(2-(trimethylsilyl)ethoxymethyl)-1H-pyrazol-3-yl)-1H-pyrazol-3-yl)-1H-pyrazole[3,4-b]pyridin-4-ylphenyl)-pyrrol-2-one (100 mg, 0.17 mmol) was dissolved in dichloromethane (5 mL), trifluoroacetic acid (2 mL) was added, and the mixture was stirred at room temperature for 8 hours. After the reaction was completed as monitored by LCMS, the reaction liquid was spun to dryness to remove the solvent. The reaction was quenched with NaHCO₃ aqueous solution (10 mL), the reaction liquid was extracted with ethyl acetate (3×30 mL), and the organic phases were combined, washed with saturated sodium chloride aqueous solution (3×30 mL), dried over anhydrous sodium sulfate, filtered and concentrated, to afford the crude product. The crude product was purified by reverse-phase column chromatography (C18, water:acetonitrile=1:1), to afford the target compound (7.7 mg, yield: 9.82%). LCMS (ESI) [M+H]⁺=462.37; ¹H NMR (400 MHz, DMSO-d6) δ 12.87 (s, 1H), 7.98-7.82 (m, 3H), 7.75 (t, J=8.6 Hz, 1H), 7.65 (dd, J=8.6, 2.0 Hz, 1H), 6.87 (s, 1H), 6.82 (d, J=2.2 Hz, 1H), 4.52 (q, J=6.3 Hz, 1H), 4.12 (d, J=12.8 Hz, 1H), 3.98 (d, J=14.1 Hz, 1H), 3.92 (t, J=7.1 Hz, 2H), 3.77 (d, J=11.3 Hz, 1H), 3.67 (dd, J=11.4, 2.7 Hz, 1H), 3.56-3.49 (m, 1H), 3.21 (td, J=12.8, 3.6 Hz, 1H), 2.58 (t, J=8.1 Hz, 2H), 2.17-2.06 (m, 2H), 1.23 (d, J=6.7 Hz, 3H).

### Examples 60-146

The following compounds of Examples 60-146 were prepared with reference to the preparation methods of Examples 1-18 and 30-33.

| | | LCMS | |
|---|---|---|---|
| Example | | (ESI) | |
| No. | Structure | [M + H]⁺ | ¹H NMR |
| 60 | 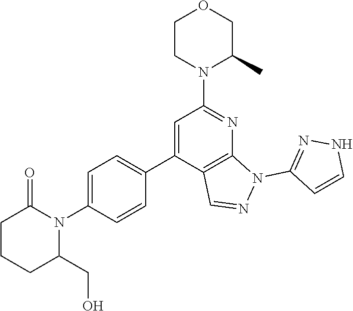 | 488.40 | ¹H NMR (400 MHz, DMSO-d6) δ 12.85 (s, 1H), 8.19 (s, 1H), 7.89 (t, J = 6.7 Hz, 3H), 7.43 (d, J = 8.4 Hz, 2H), 6.95 (s, 1H), 6.83 (s, 1H), 4.83 (t, J = 5.2 Hz, 1H), 4.67-4.52 (m, 1H), 4.18 (d, J = 12.8 Hz, 1H), 3.99 (dd, J = 11.1, 2.8 Hz, 1H), 3.88 (d, J = 4.0 Hz, 1H), 3.78 (d, J = 11.3 Hz, 1H), 3.68 (dd, J = 11.3, 2.4 Hz, 1H), 3.53 (td, J = 11.4, 2.2 Hz, 1H), 3.39 (d, J = 6.2 Hz, 1H), 3.26-3.16 (m, 1H), 2.40 (t, J = 6.4 Hz, 2H), 2.04 (dd, J = 31.3, 14.3 Hz, 3H), 1.77 (dd, J = 12.7, 6.9 Hz, 1H), 1.23 (d, J = 6.6 Hz, 3H) |
| 61 | 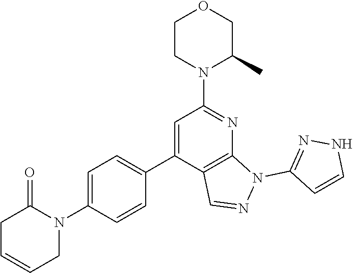 | 456.21 | |
| 62 | 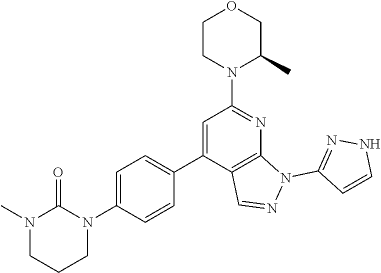 | 473.23 | |
| 63 | 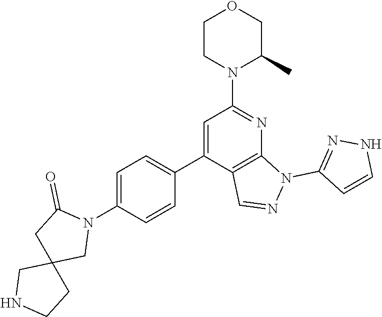 | 499.25 | |
| 64 | 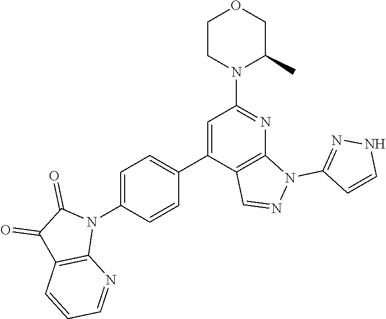 | 507.18 | |
| 65 | 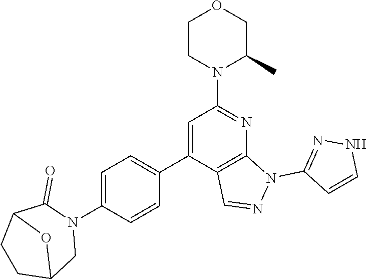 | 486.20 | ¹H NMR (400 MHz, DMSO-d6) δ 8.13 (s, 1H), 7.86 (d, J = 8.6 Hz, 3H), 7.55 (d, J = 7.9 Hz, 2H), 6.90 (s, 1H), 6.80 (s, 1H), 4.75 (s, 1H), 4.51-4.47 (m, 0H), 4.15 (s, 1H), 3.98 (s, 3H), 3.73 (s, 1H), 3.67-3.62 (m, 1H), 3.47 (s, 1H), 3.23-3.13 (m, 2H), 2.11 (s, 4H), 1.19 (d, J = 6.3 Hz, 3H) |
| 66 | 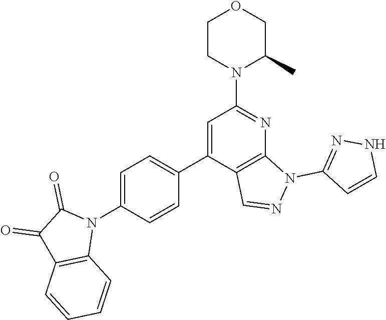 | 506.19 | |
| 67 | 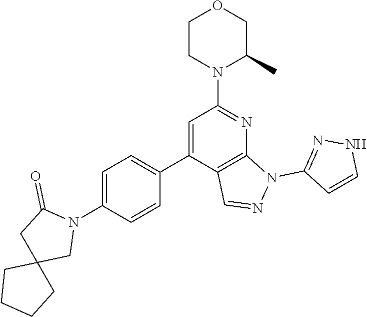 | 498.25 | |
| 68 | 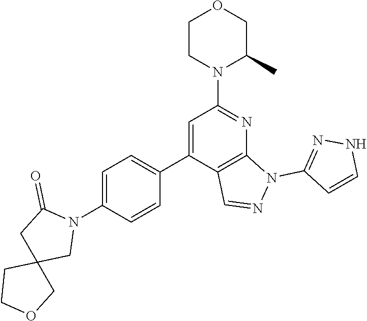 | 500.23 | |
| 69 | 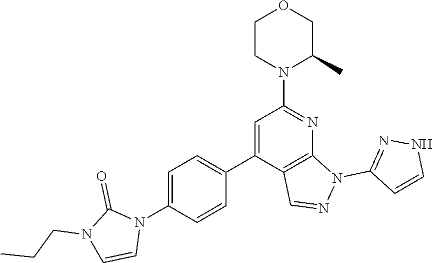 | 485.56 | |
| 70 | 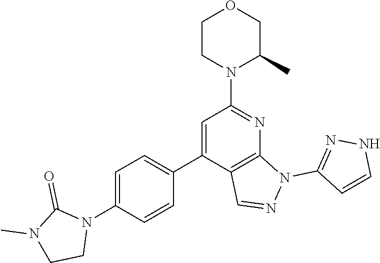 | 459.22 | |
| 71 | 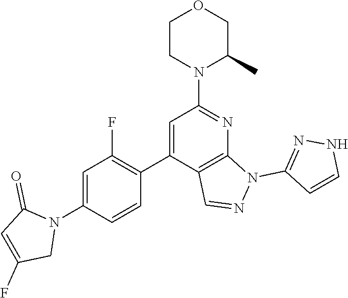 | 478.18 | |
| 72 | 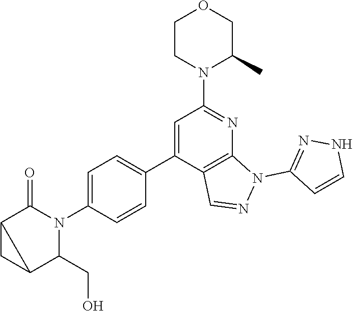 | 486.22 | |
| 73 | 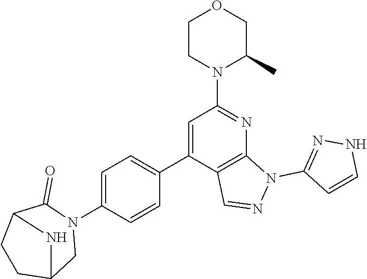 | 485.23 | |
| 74 | 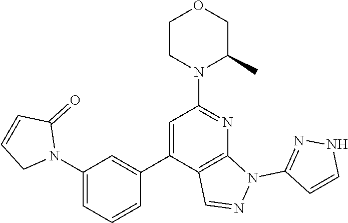 | 442.19 | |
| 75 | 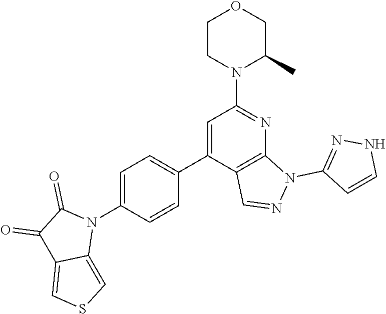 | 512.14 | |
| 76 | 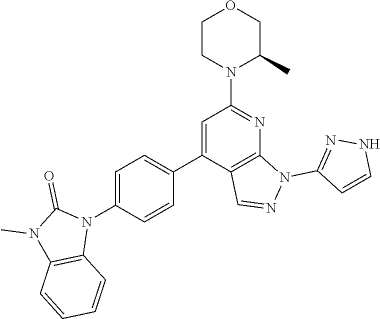 | 507.22 | |
| 77 | 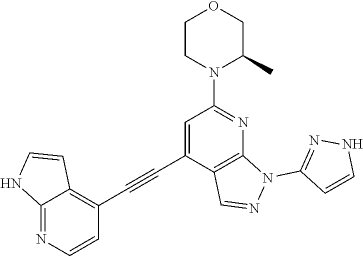 | 425.20 | ¹H NMR (400 MHz, DMSO-d6) δ 12.85 (s, 1H), 11.99 (s, 1H), 8.28 (d, J = 4.7 Hz, 1H), 8.22 (s, 1H), 7.85 (s, 1H), 7.65 (s, 1H), 7.36 (d, J = 4.8 Hz, 1H), 7.14 (s, 1H), 6.77 (s, 2H), 4.51 (s, 1H), 4.10 (d, J = 14.1 Hz, 1H), 3.96 (d, J = 12.0 Hz, 1H), 3.75 (d, J = 11.8 Hz, 1H), 3.64 (d, J = 12.8 Hz, 1H), 3.52- 3.45 (m, 2H), 1.20 (s, 3H) |
| 78 | 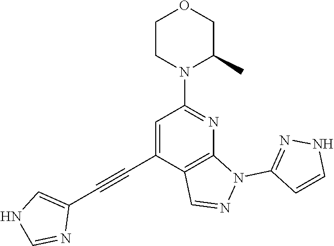 | 375.16 | ¹H NMR (400 MHz, DMSO-d6) δ 12.87 (s, 1H), 8.11 (s, 1H), 7.85 (s, 1H), 6.96 (s, 1H), 6.82 (s, 1H), 6.78 (d, J = 2.2 Hz, 1H), 4.88- 4.87 (d, J = 6.6 Hz, 2H), 4.67-4.65 (d, J = 6.6 Hz, 2H), 4.47-4.46 (d, J = 5.1 Hz, 1H), 4.10-4.07 (d, J = 12.5 Hz, 1H), 3.99-3.95 (dd, J = 11.2, 3.1 Hz, 1H), 3.77-3.74 (d, J = 11.3 Hz, 1H), 3.66-3.62 (dd, J = 11.4, 2.7 Hz, 1H), 3.52-3.46 (td, J = 11.8, 2.8 Hz, 1H), 3.21-3.14 (td, J = 12.9, 3.7 Hz, 1H), 1.20- 1.19 (d, J = 6.7 Hz, 3H) |
| 79 | 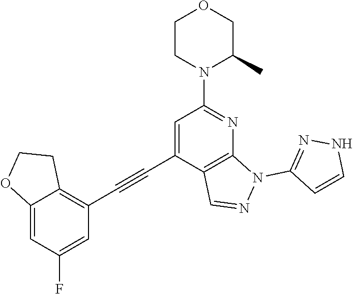 | 445.17 | |
| 80 | 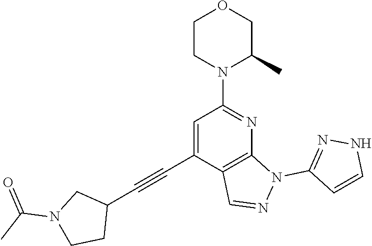 | 420.20 | ¹H NMR (400 MHz, DMSO-d6) δ 12.82 (s, 1H), 8.01 (s, 1H), 7.82 (s, 1H), 6.84 (s, 1H), 6.74 (s, 1H), 4.41 (s, 1H), 4.02 (d, J = 12.7 Hz, 1H), 3.92 (d, J = 10.5 Hz, 1H), 3.87- 3.67 (m, 2H), 3.63-3.36 (m, 6H), 3.13 (t, J = 11.2 Hz, 1H), 2.27 (dd, J = 29.8, 6.2 Hz, 1H), 2.06 (dd, J = 45.9, 7.6 Hz, 1H), 1.95 (d, J = 2.8 Hz, 3H), 1.15 (d, J = 6.5 Hz, 3H) |
| 81 | 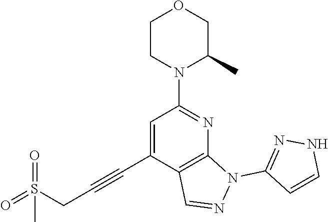 | 401.13 | |
| 82 | 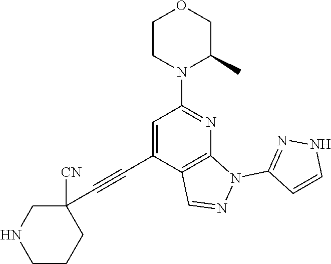 | 417.45 | ¹H NMR (400 MHz, DMSO-d6) δ 12.90- 12.79 (m, 1H), 8.27 (s, 1H), 8.06 (s, 1H), 7.82 (s, 1H), 6.98 (s, 1H), 6.74 (s, 1H), 4.45 (s, 1H), 4.06 (s, 1H), 3.92 (s, 1H), 3.71 (d, J = 11.0 Hz, 1H), 3.61 (s, 1H), 3.45 (s, 2H), 3.14 (s, 2H), 3.06 (d, J = 12.7 Hz, 1H), 2.77 (s, 1H), 2.64 (s, 1H), 2.30 (s, 1H), 2.08 (s, 1H), 1.61 (s, 2H), 1.16 (d, J = 6.3 Hz, 3H) |
| 83 | 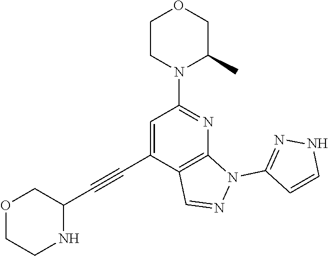 | 394.19 | |
| 84 | 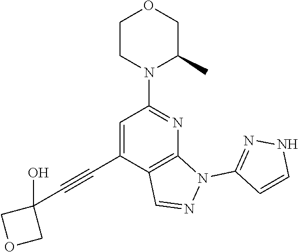 | 381.17 | ¹H NMR (400 MHz, DMSO-d6) δ 12.87 (s, 1H), 8.11 (s, 1H), 7.85 (s, 1H), 6.96 (s, 1H), 6.82 (s, 1H), 6.78 (d, J = 2.2 Hz, 1H), 4.88- 4.87 (d, J = 6.6 Hz, 2H), 4.67-4.55 (d, J = 6.6 Hz, 2H), 4.47-4.46 (d, J = 5.1 Hz, 1H), 4.10-4.07 (d, J = 12.5 Hz, 1H), 3.99-3.95 (dd, J = 11.2, 3.1 Hz, 1H), 3.77-3.74 (d, J = 11.3 Hz, 1H), 3.66-3.62 (dd, J = 11.4, 2.7 Hz, 1H), 3.52-3.46 (td, J = 11.8, 2.8 Hz, 1H), 3.21-3.14 (td, J = 12.9, 3.7 Hz, 1H), 1.20- 1.19 (d, J = 6.7 Hz, 3H) |
| 85 | 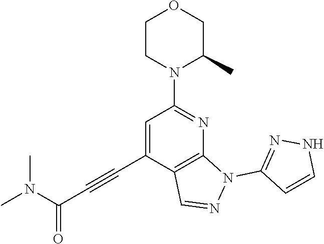 | 380.18 | |
| 86 | 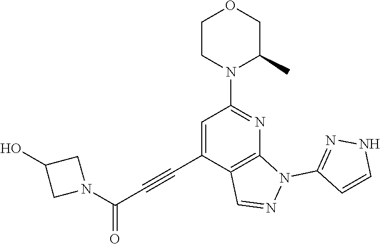 | 408.17 | ¹H NMR (400 MHz, DMSO-d6) δ 12.91 (s, 1H), 8.12 (s, 1H), 7.87 (s, 1H), 7.15 (s, 1H), 6.77 (d, J = 2.1 Hz, 1H), 5.93 (s, 1H), 4.58- 4.56 (d, J = 6.0 Hz, 2H), 4.50-4.48 (d, J = 5.6 Hz, 1H), 4.24-4.19 (dd, J = 11.5, 5.7 Hz, 1H), 4.11-4.08 (m, 2H), 4.02-3.93 (m, 1H), 3.77-3.71 (dd, J = 17.1, 6.8 Hz, 2H), 3.66- 3.63 (dd, J = 11.4, 2.7 Hz, 1H), 3.52-3.47 (dd, J = 11.7, 9.2 Hz, 1H), 3.23-3.15 (td, J = 13.1, 3.7 Hz, 1H), 1.21-1.19 (d, J = 6.7 Hz, 3H) |
| 87 | 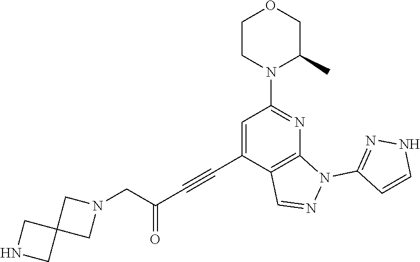 | 447.22 | |
| 88 | 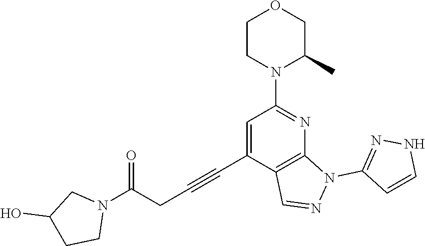 | 436.20 | |
| 89 | 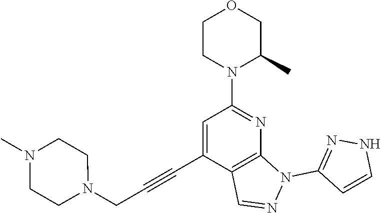 | 421.20 | 1H NMR (400 MHz, CD₃OD) δ 8.03 (s, 1H), 7.72 (s, 1H), 6.89 (s, 1H), 6.84 (s, 1H), 4.52 (d, J = 41.9 Hz, 2H), 4.07 (d, J = 13.6 Hz, 1H), 4.00 (dd, J = 11.7, 3.6 Hz, 1H), 3.76 (dt, J = 11.3, 7.3 Hz, 2H), 3.69 (s, 2H), 3.60 (td, J = 11.8, 3.0 Hz, 1H), 2.67 (d, J = 75.9 Hz, 8H), 2.31 (s, 3H), 1.28 (d, J = 6.7 Hz, 3H) |
| 90 | 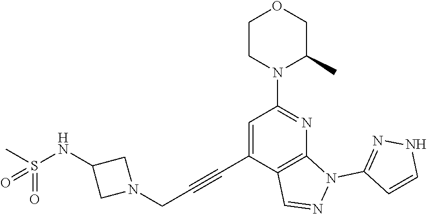 | 471.18 | |
| 91 | 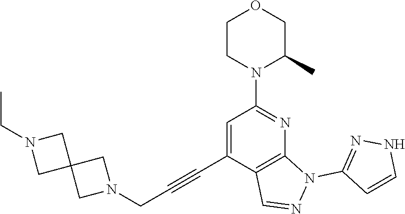 | 447.25 | |
| 92 | 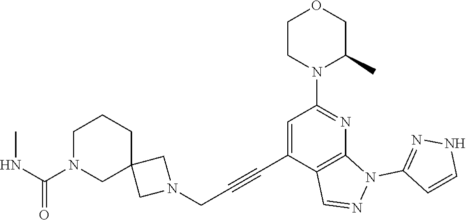 | 504.28 | |
| 93 | 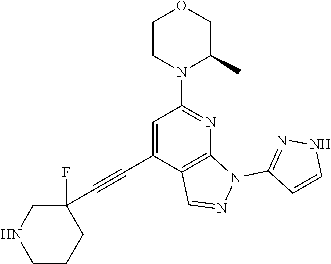 | 410.20 | |
| 94 | 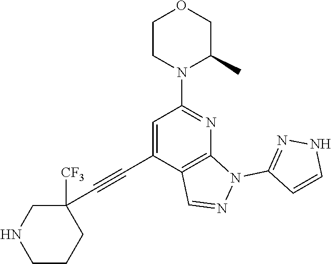 | 460.20 | |
| 95 | 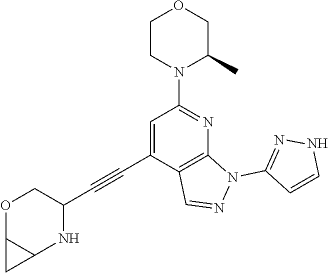 | 406.19 | |
| 96 | 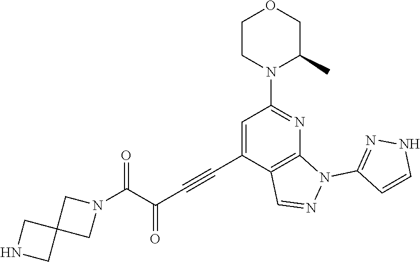 | 461.20 | |
| 97 | 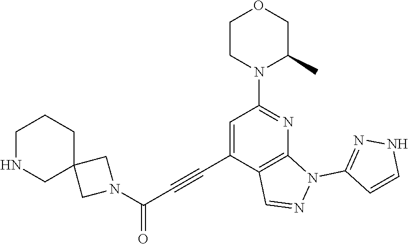 | 461.23 | |
| 98 | 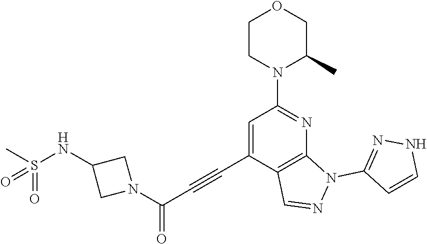 | 485.16 | |
| 99 | 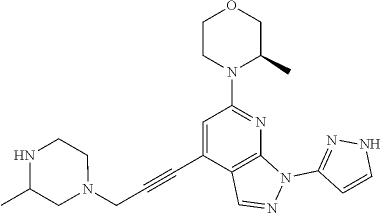 | 421.24 | |
| 100 | 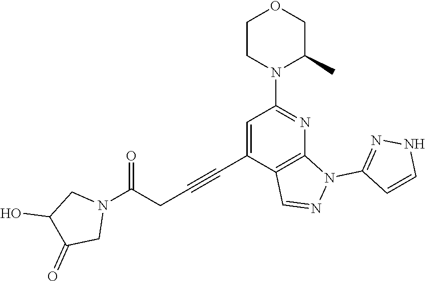 | 450.18 | |
| 101 | 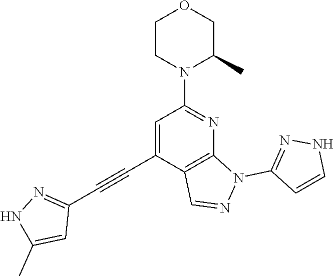 | 389.18 | |
| 102 | 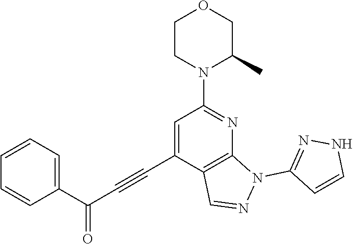 | 413.17 | |
| 103 | 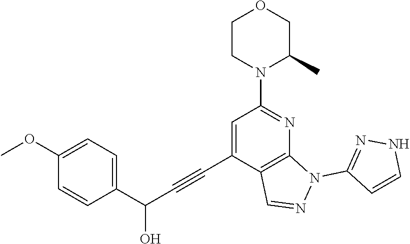 | 445.19 | |
| 104 | 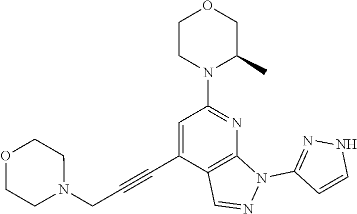 | 408.22 | |
| 105 | 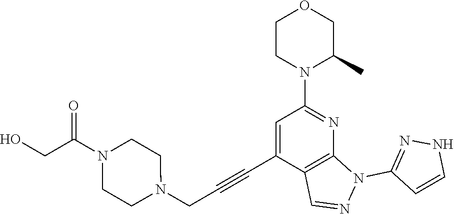 | 465.23 | |
| 106 | 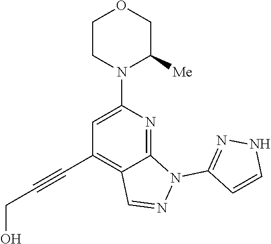 | 339.15 | |
| 107 | 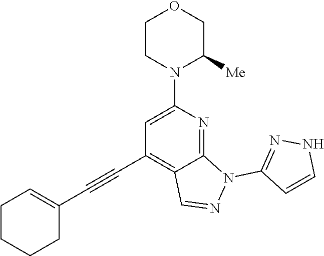 | 389.20 | |
| 108 | 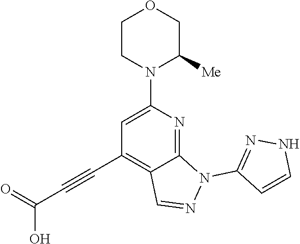 | 353.13 | |
| 109 | 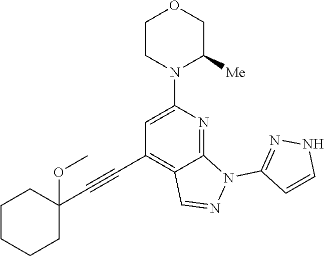 | 421.23 | |
| 110 | 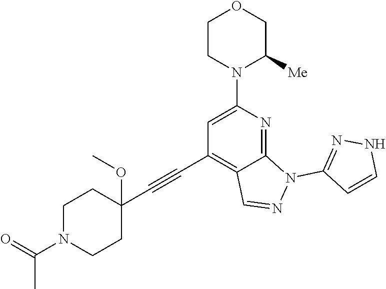 | 464.23 | |
| 111 | 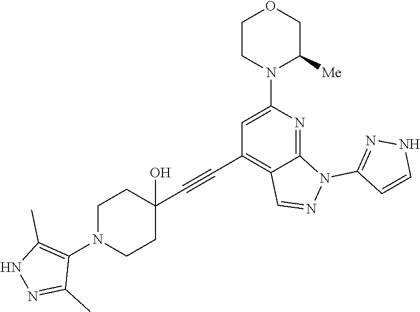 | 502.26 | |
| 112 | 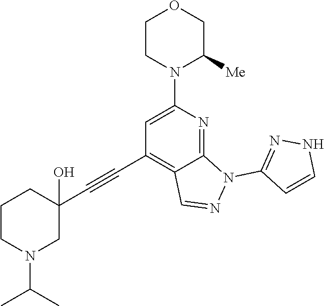 | 450.25 | |
| 113 | 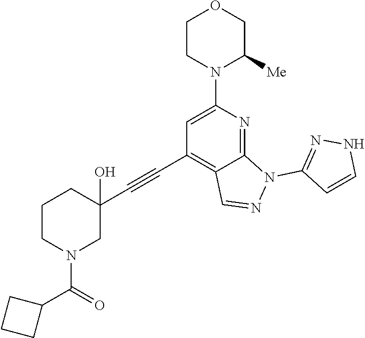 | 490.25 | |
| 114 | 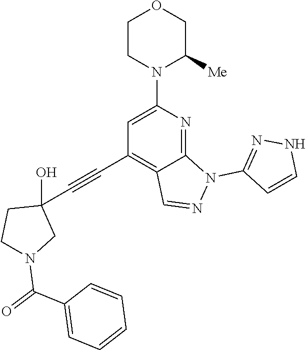 | 498.22 | |
| 115 | 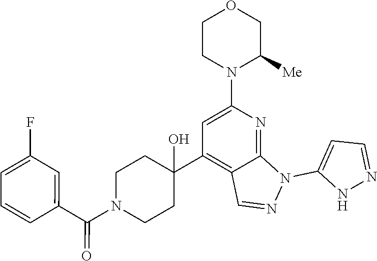 | 506.22 | |
| 116 | 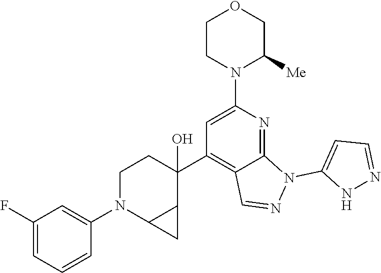 | 490.23 | |
| 117 | 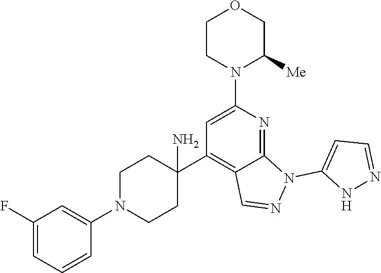 | 477.24 | |
| 118 | 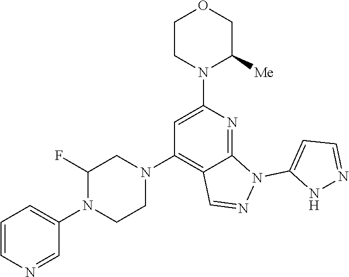 | 464.22 | |
| 119 | 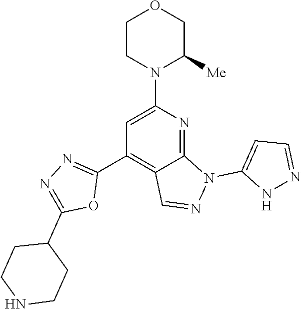 | 436.21 | |
| 120 | 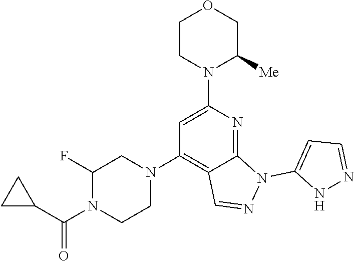 | 455.22 | |
| 121 | 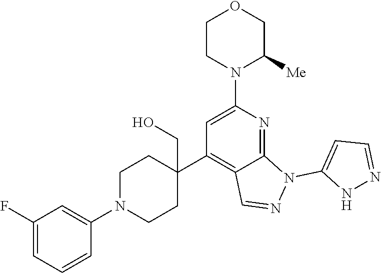 | 492.24 | |
| 122 | 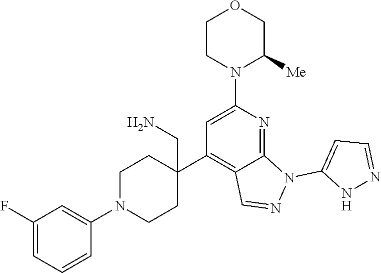 | 491.26 | |
| 123 | 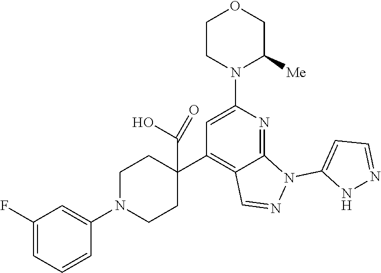 | 463.15 | |
| 124 | 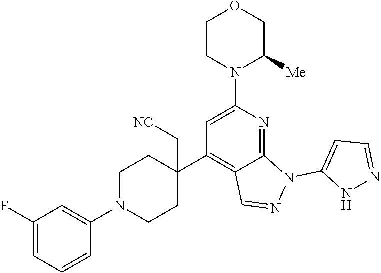 | 501.24 | |
| 125 | 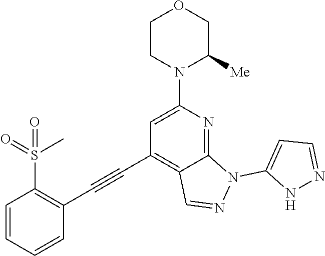 | 463.15 | |
| 126 | 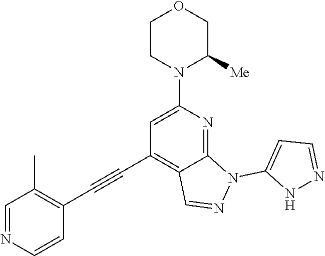 | 400.18 | |
| 127 | 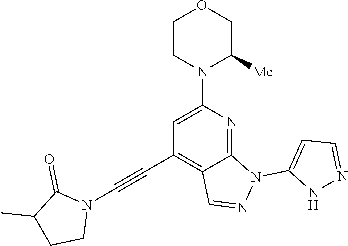 | 406.19 | |
| 128 | 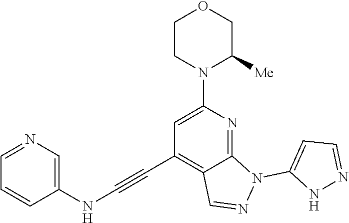 | 401.18 | |
| 129 | 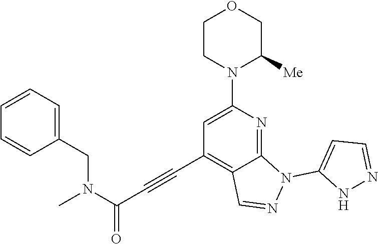 | 456.22 | |
| 130 | 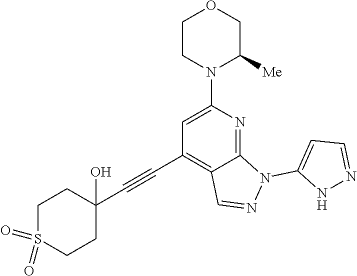 | 457.16 | |
| 131 | 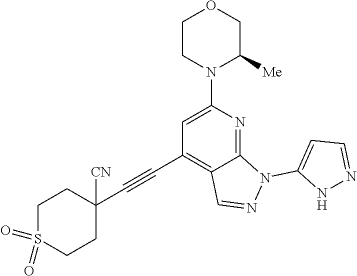 | 466.16 | |
| 132 | 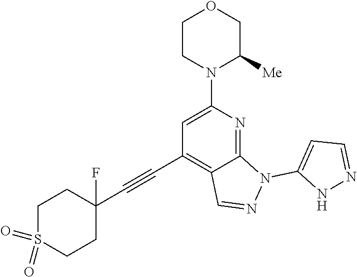 | 459.15 | |
| 133 | 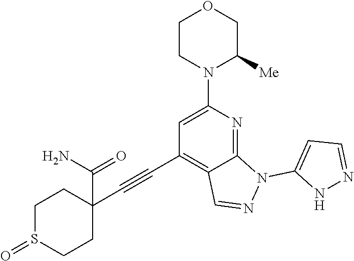 | 468.17 | |
| 134 | 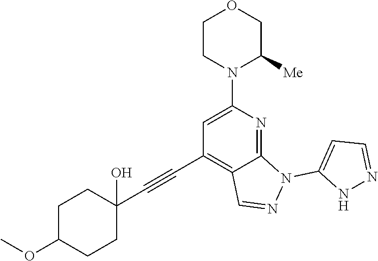 | 437.22 | |
| 135 | 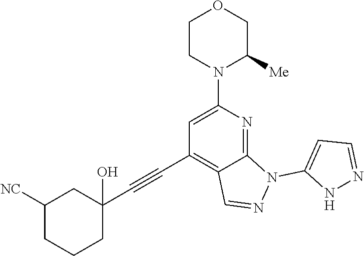 | 432.22 | |
| 136 | 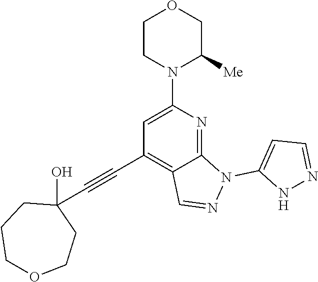 | 423.21 | |
| 137 | 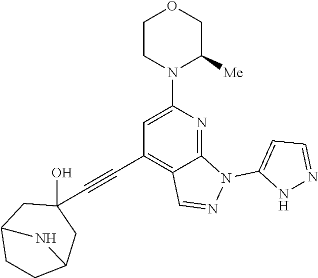 | 434.22 | |
| 138 | 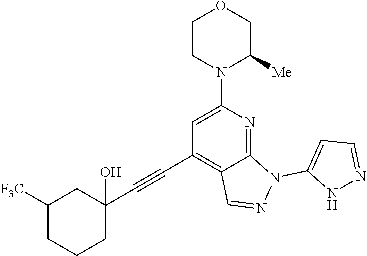 | 475.20 | |
| 139 | 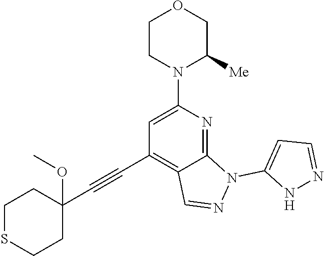 | 439.18 | |
| 140 | 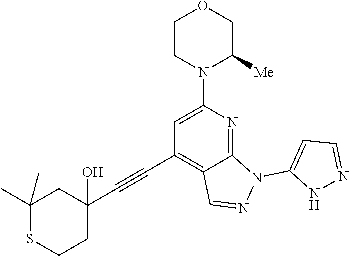 | 453.20 | |
| 141 | 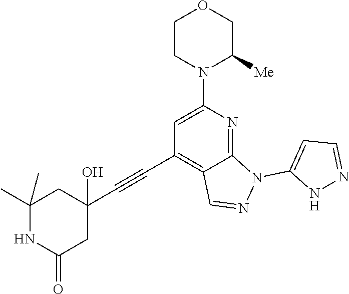 | 464.14 | |
| 142 | 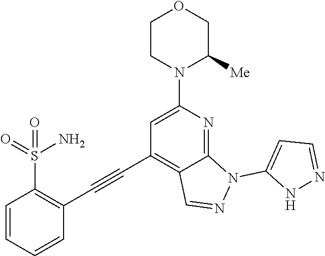 | 464.14 | |
| 143 | 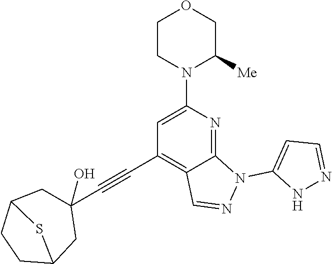 | 451.18 | |
| 144 | 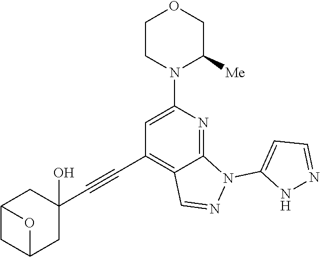 | 421.19 | |
| 145 | 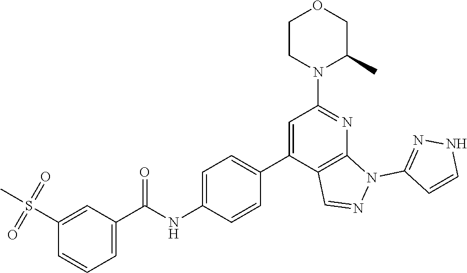 | 558.15 | ¹H NMR (400 MHz, DMSO-d6) δ 12.82 (s, 1H), 10.74 (s, 1H), 8.49 (s, 1H), 8.32 (d, J = 7.4 Hz, 1H), 8.20 (s, 1H), 8.14 (d, J = 7.8 Hz, 1H), 7.99 (d, J = 8.3 Hz, 2H), 7.90 (d, J = 8.4 Hz, 2H), 7.82 (d, J = 9.2 Hz, 2H), 6.92 (s, 1H), 6.81 (s, 1H), 4.57 (s, 1H), 4.13 (d, J = 12.0 Hz, 1H), 3.97 (d, J = 10.5 Hz, 1H), 3.76 (d, J = 11.0 Hz, 1H), 3.66 (d, J = 12.1 Hz, 1H), 3.49 (d, J = 11.9 Hz, 1H), 3.29 (s, 4H), 3.21 (d, J = 11.4 Hz, 1H), 1.20 (d, J = 6.0 Hz, 3H) |
| 146 | 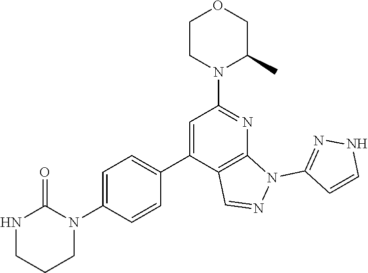 | 459.20 | ¹H NMR (400 MHz, CD₃OD) δ 8.10 (s, 1H), 7.77 (t, J = 10.5 Hz, 3H), 7.49 (d, J = 7.5 Hz, 2H), 6.97 (s, 1H), 6.85 (s, 1H), 4.59 (s, 1H), 4.17 (d, J = 12.9 Hz, 1H), 4.04 (d, J = 9.3 Hz, 1H), 3.80 (dd, J = 11.7, 7.1 Hz, 4H), 3.64 (s, 2H), 3.41 (t, J = 5.8 Hz, 2H), 2.12 (s, 2H), 1.32 (d, J = 6.3 Hz, 3H) |

### Example 147

### Preparation of (R)-4-ethynyl-1-(6-(3-methylmorpholinyl)-1-(1H-pyrazolyl-3-yl)-1H-pyrazolyl[3,4-b]pyridin-4-yl)cyclohexanol

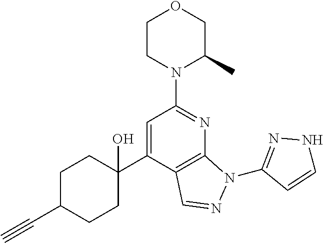 [SMILES: C#CC1CCC(O)(c2cc(N3CCOC[C@H]3C)nc3c2cnn3-c2cc[nH]n2)CC1]

### Step 1: Preparation of 4-((tert-butyldiphenylsilyloxy)methyl)cyclohexanone

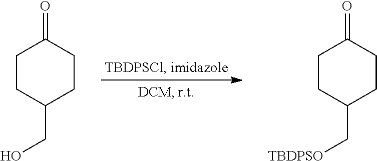

4-(Hydroxymethyl)cyclohexanone (900.0 mg, 7.02 mmol, 1 equiv.) and imidazole (1.4 g, 21.07 mmol, 3 equiv.) were dissolved in dichloromethane (20 mL), tert-butyl diphenylsilyl chloride (2.9 g, 10.53 mmol, 1.5 equiv.) was added under ice bath, and the mixture was heated to room temperature and stirred for 2 hours. After the reaction was completed as monitored by TLC, liquid separation was performed by adding water and dichloromethane, the organic phases were combined, dried, filtered and concentrated, and the residue was purified by column chromatography (petroleum ether:ethyl acetate=50:1), to afford the target compound (1.5 g, yield: 58.2%). ¹H NMR (400 MHz, DMSO-d6) δ 7.65 (d, J=3.6 Hz, 1H), 7.58 (d, J=5.9 Hz, 4H), 7.41 (d, J=6.7 Hz, 4H), 7.35 (s, 1H), 3.53 (d, J=5.8 Hz, 2H), 2.40-2.31 (m, 2H), 2.15 (d, J=14.0 Hz, 2H), 1.98 (s, 3H), 1.40-1.31 (m, 2H), 0.97 (s, 9H).

### Step 2: Preparation of 4-((tert-butyldiphenylsilyl)oxy)methyl)cyclohex-1-en-1-yl trifluoromethanesulfonate

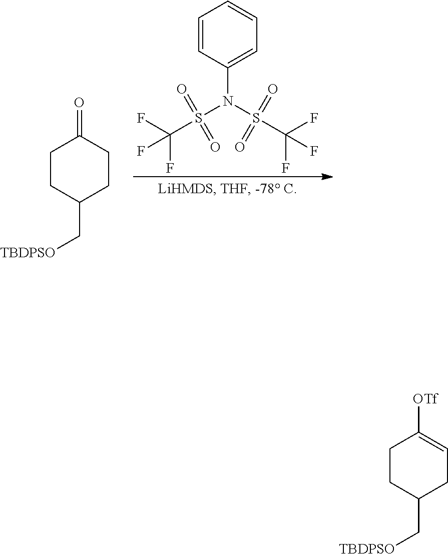

4-((Tert-butyldiphenylsilyloxy)methyl)cyclohexanone (700.0 mg, 1.91 mmol, 1 equiv.) was dissolved in tetrahydrofuran (5 mL), lithium hexamethyldisilazide (2.4 mL, 14.65 mmol, 1.2 equiv.) was added at −78° C., and the mixture was stirred at −78° C. for 45 minutes. 1,1,1-Trifluoro-N-phenyl-N-((trifluoromethyl)sulfonyl)methanesulfonamide (750.4 mg, 2.10 mmol, 1.1 equiv.) dissolved in tetrahydrofuran was added, and then the resulting mixture was stirred at room temperature for 3 hours. After the raw materials were reacted completely as monitored by TLC, liquid separation was performed by adding ethyl acetate and water, the organic phases were combined, dried, filtered and concentrated, and the residue was purified by column chromatography (petroleum ether:ethyl acetate=100:1), to afford the target compound (560.0 mg, yield: 58.8%). ¹H NMR (400 MHz, DMSO-d6) δ 7.57 (d, J=7.0 Hz, 4H), 7.41 (d, J=7.1 Hz, 5H), 5.85 (s, 1H), 3.54 (d, J=6.1 Hz, 2H), 2.24 (d, J=18.9 Hz, 2H), 2.02-1.70 (m, 4H), 1.48 (s, 1H), 0.97 (s, 9H).

### Step 3: Preparation of tert-butyldiphenyl((4-(4,4,5,5-tetramethyl-1,3,2-dioxaborolan-2-yl)cyclohex-3-en-1-yl)methoxy)silane

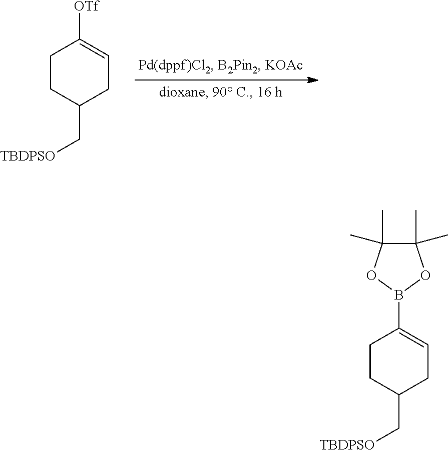

4-((Tert-butyldiphenylsilyl)oxy)methyl)cyclohex-1-en-1-yl trifluoromethanesulfonate (560.0 mg, 1.12 mmol, 1 equiv.), bis(pinacolato)diboron (427.7 mg, 1.68 mmol, 1.5 equiv.), [1,1′-bis(diphenylphosphino)ferrocene]dichloropalladium (II) (82.4 mg, 0.11 mmol, 0.1 equiv.) and potassium acetate (330.6 mg, 3.37 mmol, 3 equiv.) were dissolved in 1, 4-dioxane (10 mL), and the mixture was stirred at 90° C. for 16 hours after nitrogen replacement was performed three times. After the raw materials were reacted completely as monitored by TLC, the reaction liquid was filtered with diatomaceous earth, and the filtrate was subjected to liquid separation by adding ethyl acetate and water. The organic phases were combined, dried, filtered and concentrated, and the residue was purified by column chromatography (petroleum ether:ethyl acetate=100:1), to afford the target compound (340.0 mg, yield: 63.2%). ¹H NMR (400 MHz, DMSO-d6) δ 7.57 (d, J=6.5 Hz, 4H), 7.41 (d, J=6.5 Hz, 6H), 5.29 (s, 1H), 3.50 (d, J=5.9 Hz, 2H), 2.07 (d, J=19.8 Hz, 1H), 1.99-1.92 (m, 4H), 1.70 (s, 4H), 1.42 (s, 1H), 1.15 (s, 12H), 0.96 (s, 9H).

### Step 4: Preparation of (3R)-4-(4-(tert-butyldiphenylsilyloxy)methyl)cyclohex-1-en-1-yl)-1-(1-(2-(trimethylsilyloxy)ethoxy)methyl)-1H-pyrazol-5-yl)-1H-pyrazolyl[3,4-b]pyridin-6-yl)-3-methylmorpholine

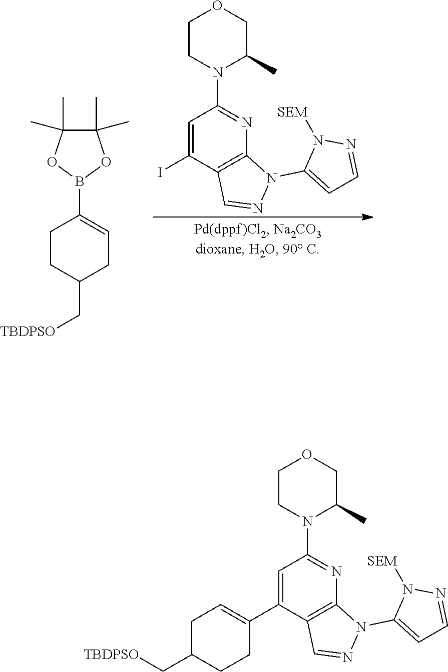

Tert-butyldiphenyl((4-(4,4,5,5-tetramethyl-1,3,2-dioxaborolan-2-yl)cyclohex-3-en-1-yl)methoxy)silane (340.0 mg, 0.36 mmol, 1 equiv.), (R)-4-(4-iodo-1-(1-((2-(trimethylsilyl)ethoxy)methyl)-1H-pyrazol-5-yl)-1H-pyrazole[3,4-b]pyridin-6-yl)-3-methylmorpholine (385.6 mg, 0.36 mmol, 1 equiv.), sodium carbonate (75.6 mg, 0.71 mmol, 2 equiv.) and [1,1′-bis(diphenylphosphino)ferrocene]dichloropalladium (II) (26.1 mg, 0.04 mmol, 0.1 equiv.) were dissolved in 1, 4-dioxane (6 mL) and water (3 mL), and the mixture was stirred at 90° C. for 2 hours. After the reaction was completed as monitored by LCMS, the reaction liquid was filtered with diatomaceous earth, and the filtrate was subjected to liquid separation by adding ethyl acetate and water. The organic phases were combined, dried, filtered and concentrated, and the residue was purified by prepTLC (petroleum ether:ethyl acetate=3:1), to afford the target compound (260.0 mg, yield: 47.7%). ¹H NMR (400 MHz, CDCl₃) δ 8.04 (d, J=5.1 Hz, 1H), 7.71-7.60 (m, 5H), 7.40 (t, J=8.3 Hz, 6H), 6.59 (s, 2H), 6.42 (s, 1H), 6.36 (s, 1H), 5.71 (t, J=12.6 Hz, 3H), 4.31 (d, J=21.8 Hz, 1H), 3.97 (t, J=14.3 Hz, 3H), 3.74 (dd, J=29.1, 14.3 Hz, 3H), 3.63 (d, J=5.8 Hz, 2H), 3.57 (s, 1H), 3.40 (d, J=8.0 Hz, 3H), 3.25 (d, J=12.9 Hz, 1H), 1.26 (dd, J=11.4, 5.9 Hz, 9H), 1.06 (s, 9H), 0.78-0.70 (m, 3H), −0.17 (d, J=4.4 Hz, 14H).

### Step 5: Preparation of (R)-4-((tert-butyldiphenylsilyl)oxy)methyl)-1-(6-(3-methylmorpholinyl)-1-(1-((2-(trimethylsilyl)ethoxy)methyl)-1H-pyrazolyl-5-yl)-1H-pyrazolyl[3,4-b]pyridin-4-yl)cyclohexanol

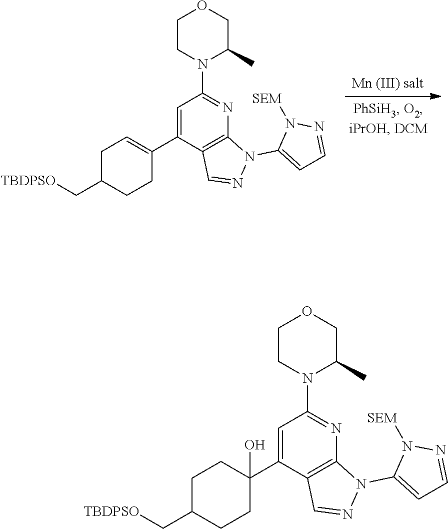

(3R)-4-(4-(Tert-butyldiphenylsilyloxy)methyl)cyclohex-1-en-1-yl)-1-(1-(2-(trimethylsilyloxy)ethoxy)methyl)-1H-pyrazol-5-yl)-1H-pyrazolyl[3,4-b]pyridin-6-yl)-3-methylmorpholine (260.0 mg, 0.34 mmol, 1 equiv.) was dissolved in isopropanol (3.5 mL) and dichloromethane (0.5 mL), tris(2,2,6,6-tetramethyl-3,5-heptanedionato)manganese (41.4 mg, 0.07 mmol, 0.2 equiv.) and phenylsilane (73.7 mg, 0.68 mmol, 2 equiv.) were added under ice bath, and the mixture was stirred at room temperature for 2 hours after oxygen replacement was performed three times. After the raw materials were reacted completely as monitored by LCMS, the reaction liquid was subjected to liquid separation by adding ethyl acetate and water. The organic phases were combined, dried, filtered and concentrated, and the residue was purified by prepTLC (petroleum ether:ethyl acetate=2:1), to afford the target compound (200.0 mg, yield: 75.1%). LCMS (ESI) [M+H]⁺=781.40.

### Step 6: Preparation of (R)-4-(hydroxymethyl)-1-(6-(3-methylmorpholinyl)-1-(1-((2-(trimethylsilyl)ethoxy)methyl)-1H-pyrazol-5-yl)-1H-pyrazole[3,4-b]pyridin-4-yl)cyclohexanol

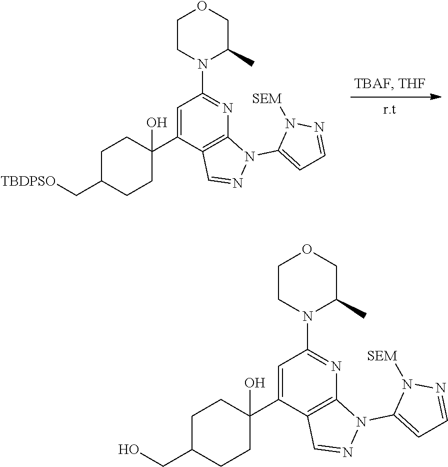

(R)-4-((Tert-butyldiphenylsilyl)oxy)methyl)-1-(6-(3-methylmorpholinyl)-1-(1-((2-(trimethylsilyl)ethoxy)methyl)-1H-pyrazolyl-5-yl)-1H-pyrazolyl[3,4-b]pyridin-4-yl)cyclohexanol (200.0 mg, 0.26 mmol, 1 equiv.) was dissolved in tetrahydrofuran (2 mL), 1 M tetrabutylammonium fluoride tetrahydrofuran solution (2 mL) was added, and the mixture was stirred overnight at room temperature. After the reaction was completed as monitored by LCMS, the reaction was quenched with saturated ammonium chloride aqueous solution, and the reaction liquid was extracted with ethyl acetate. The organic phases were combined, dried, filtered and concentrated, and the residue was purified by prepTLC (petroleum ether:ethyl acetate=1:1), to afford the target compound (130.0 mg, yield: 93.55%). LCMS (ESI) [M+H]⁺=543.55.

### Step 7: Preparation of (R)-4-hydroxy-4-(6-(3-methylmorpholinyl)-1-(1-((2-(trimethylsilyl)ethoxy)methyl)-1H-pyrazol-5-yl)-1H-pyrazole[3,4-b]pyridin-4-yl)cyclohexanecarbaldehyde

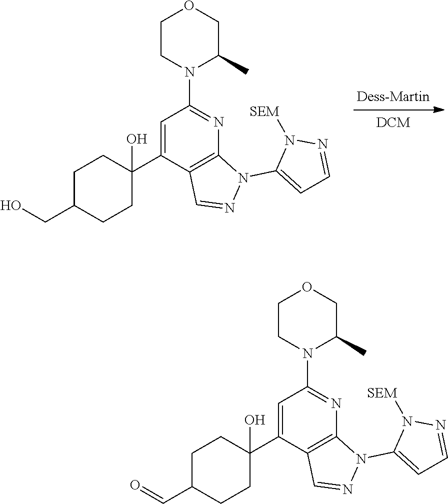

(R)-4-(Hydroxymethyl)-1-(6-(3-methylmorpholinyl)-1-(1-((2-(trimethylsilyl)ethoxy)methyl)-1H-pyrazol-5-yl)-1H-pyrazole[3,4-b]pyridin-4-yl)cyclohexanol (130.0 mg, 0.24 mmol, 1 equiv.) was dissolved in dichloromethane (4 mL), Dess-Martin periodinane (203.1 mg, 0.48 mmol, 2 equiv.) was added under ice bath, and the mixture was heated to room temperature and stirred for 2 hours. After the reaction was completed as monitored by LCMS, the reaction liquid was filtered with diatomaceous earth, and the filtrate was quenched with saturated sodium thiosulfate aqueous solution and extracted with ethyl acetate. The organic phases were combined, dried, filtered and concentrated, and the residue was purified by prepTLC (petroleum ether:ethyl acetate=1:1), to afford the target compound (100.0 mg, yield: 75.1%). LCMS (ESI) [M+H]⁺=541.30.

### Step 8: Preparation of ((R)-4-ethynyl-1-(6-(3-methylmorpholinyl)-1-(1-((2-(trimethylsilyl)ethoxy)methyl)-1H-pyrazolyl-5-yl)-1H-pyrazolyl[3,4-b]pyridin-4-yl)cyclohexanol

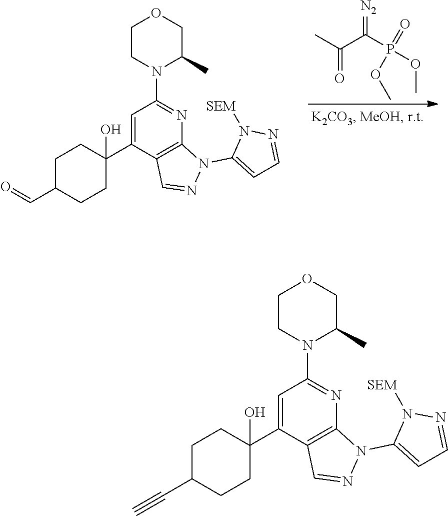

(R)-4-Hydroxy-4-(6-(3-methylmorpholinyl)-1-(1-((2-(trimethylsilyl)ethoxy)methyl)-1H-pyrazol-5-yl)-1H-pyrazole[3,4-b]pyridin-4-yl)cyclohexanecarbaldehyde (100.0 mg, 0.18 mmol, 1 equiv.), dimethyl (1-diazo-2-oxopropyl)phosphonate (42.6 mg, 0.22 mmol, 1.2 equiv.) and potassium carbonate (51.1 mg, 0.37 mmol, 2 equiv.) were dissolved in methanol (3 mL), and the mixture was stirred overnight at room temperature. After the reaction was completed as monitored by LCMS, the reaction liquid was subjected to liquid separation by adding ethyl acetate and water. The organic phases were combined, dried, filtered and concentrated, and the residue was purified by prepTLC (dichloromethane:methanol=15:1), to afford the target compound (87.0 mg, yield: 87.6%). LCMS (ESI) [M+H]⁺=537.35.

### Step 9: Preparation of (R)-4-ethynyl-1-(6-(3-methylmorpholinyl)-1-(1H-pyrazolyl-3-yl)-1H-pyrazolyl[3,4-b]pyridin-4-yl)cyclohexanol

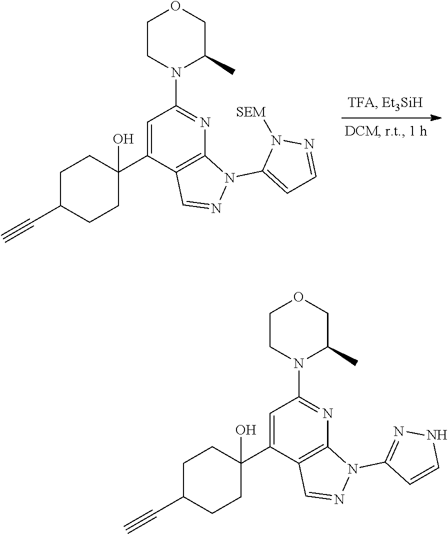

((R)-4-Ethynyl-1-(6-(3-methylmorpholinyl)-1-(1-((2-(trimethylsilyl)ethoxy)methyl)-1H-pyrazolyl-5-yl)-1H-pyrazolyl[3,4-b]pyridin-4-yl)cyclohexanol (90.0 mg, 0.17 mmol, 1 equiv.) was dissolved in trifluoroacetic acid (2 mL) and dichloromethane (2 mL), triethylsilane (0.2 mL) was added, and the mixture was stirred at room temperature for 1 hour. After the reaction was completed as monitored by LCMS, the reaction liquid was adjusted to be alkaline with saturated sodium bicarbonate aqueous solution and extracted with ethyl acetate, the organic phases were combined, dried, filtered and concentrated, and the residue was purified by prepTLC (dichloromethane:methanol=10:1), to afford the target compound (46.2 mg, yield: 67.7%). LCMS (ESI) [M+H]⁺=407.20; ¹H NMR (400 MHz, DMSO-d6) δ 12.76 (s, 1H), 8.24 (s, 1H), 7.80 (s, 1H), 6.77 (d, J=12.5 Hz, 2H), 5.23 (s, 1H), 4.40 (s, 1H), 4.03-3.90 (in, 2H), 3.73 (d, J=10.9 Hz, 1H), 3.61 (d, J=10.7 Hz, 1H), 3.46 (t, J=11.3 Hz, 1H), 3.13 (t, J=11.8 Hz, 1H), 2.86 (s, 1H), 2.54 (s, 1H), 1.96 (d, J=12.5 Hz, 2H), 1.85 (dd, J=24.4, 12.5 Hz, 2H), 1.76 (s, 2H), 1.67 (d, J=12.1 Hz, 2H), 1.15 (d, J=6.1 Hz, 3H).

### Examples 148-200

The following compounds of Examples 148-200 were prepared with reference to the preparation methods of Examples 1-18, 30-33 and 147.

| Example | | LCMS (ESI) | |
|---|---|---|---|
| No. | Structure | [M + H]⁺ | ¹H NMR |
| 148 | 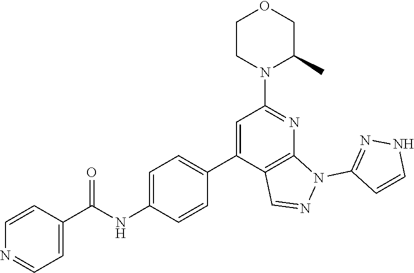 | 481.20 | ¹H NMR (400 MHz, DMSO-d6)δ 12.82 (s, 1H), 10.71 (s, 1H), 8.79 (d, J = 4.4 Hz, 2H), 8.20 (s, 1H), 7.98 (d, J = 8.6 Hz, 2H), 7.91 (s, 1H), 7.88 (d, J = 4.4 Hz, 3H), 7.84 (s, 1H), 6.91 (s, 1H), 6.81 (s, 1H), 4.55 (s, 1H), 4.13 (d, J = 14.1 Hz, 1H), 3.96 (d, J = 10.2 Hz, 1H), 3.75 (d, J = 11.6 Hz, 1H), 3.65 (d, J = 9.7 Hz, 1H), 3.50 (t, J = 11.0 Hz, 1H), 3.19 (s, 1H), 1.20 (d, J = 6.4 Hz, 3H) |
| 149 | 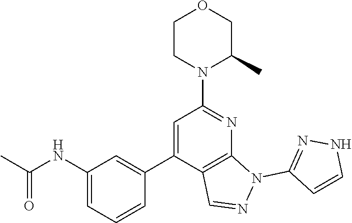 | 433.15 | ¹H NMR (400 MHz, DMSO-d6) δ 12.83 (s, 1H), 10.14 (s, 1H), 8.15 (s, 1H), 8.06 (s, 1H), 7.84 (s, 1H), 7.72 (d, J = 7.0 Hz, 1H), 7.47 (d, J = 10.4 Hz, 2H), 6.88 (s, 1H), 6.81 (s, 1H), 4.52 (s, 1H), 4.11 (d, J = 13.3 Hz, 1H), 3.95 (d, J = 9.7 Hz, 1H), 3.74 (d, J = 11.4 Hz, 1H), 3.64 (d, J = 10.5 Hz, 1H), 3.49 (t, J = 10.8 Hz, 1H), 3.19 (t, J = 11.6 Hz, 1H), 2.06 (s, 3H), 1.19 (d, J = 6.1 Hz, 3H) |
| 150 | 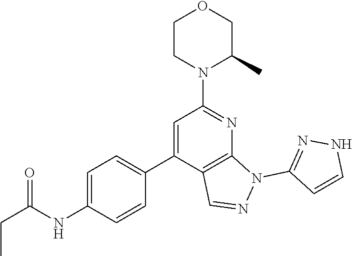 | 432.38 | ¹H NMR (400 MHz, DMSO-d6) δ 12.83 (s, 1H), 10.12 (s, 1H), 8.21 (s, 1H), 7.89-7.76 (m, 5H), 6.90 (s, 1H), 6.83 (d, J = 2.2 Hz, 1H), 4.58-4.57 (t, J = 7.3 Hz, 1H), 4.17-4.14 (d, J = 12.0 Hz, 1H), 4.01-3.97 (dd, J = 11.2, 3.2 Hz, 1H), 3.79- 3.78 (d, J = 11.2 Hz, 1H), 3.69-3.66 (dd, J = 11.3, 2.7 Hz, 1H), 3.56-3.49 (m, 2H), 3.25-3.18 (m, 1H), 2.41-2.35 (q, J = 7.5 Hz, 2H), 1.23- 1.21 (d, J = 6.7 Hz, 3H), 1.13-1.10 (t, J = 7.5 Hz, 3H) |
| 151 | 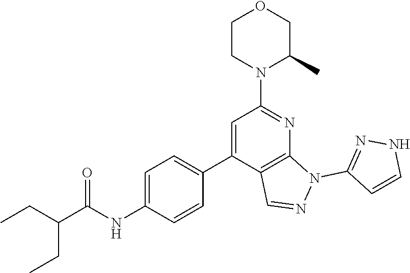 | 474.41 | ¹H NMR (400 MHz, DMSO-d6) δ 12.87 (s, 1H), 10.13 (s, 1H), 8.21 (s, 1H), 7.87-7.82 (m, 5H), 6.89 (s, 1H), 6.83 (d, 1H), 4.60-4.55 (m, 1H), 4.17-4.13 (d, J = 12.6 Hz, 1H), 4.00-3.98 (d, J = 11.3 Hz, 1H), 3.80-3.65 (m, 2H), 3.56-3.50 (d, J = 11.8 Hz, 1H), 3.24-3.19 (d, J = 12.3 Hz, 1H), 2.30-2.25 (m, 1H), 1.63-1.56 (m, 2H), 1.50- 1.45 (m, 2H), 1.23-1.22 (d, J = 6.7 Hz, 3H), 0.90-0.86 (t, J = 7.4 Hz, 6H) |
| 152 | 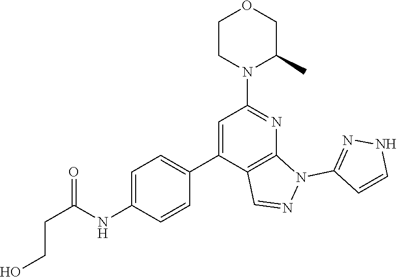 | 448.40 | ¹H NMR (400 MHz, DMSO-d6) δ 12.82 (s, 1H), 10.14 (s, 1H), 8.17 (s, 1H), 7.83 (s, 1H), 7.81 (s, 4H), 6.87 (s, 1H), 6.81 (s, 1H), 4.71 (t, J = 5.2 Hz, 1H), 4.54 (d, J = 4.4 Hz, 1H), 4.12 (d, J = 14.2 Hz, 1H), 3.96 (d, J = 9.8 Hz, 1H), 3.74- 3.70 (m, 2H), 3.66 (s, 1H), 3.48 (d, J = 9.5 Hz, 1H), 3.23-3.12 (m, 2H), 2.00-1.92 (m, 2H), 1.19 (s, 3H) |
| 153 | 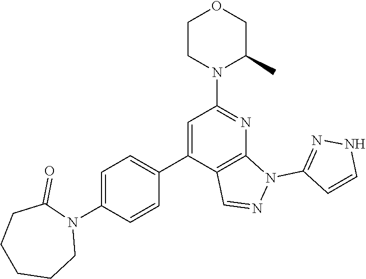 | 472.50 | ¹H NMR (400 MHz, DMSO-d6) δ 8.13 (s, 1H), 7.83 (d, J = 8.3 Hz, 3H), 7.39 (d, J = 8.7 Hz, 2H), 6.89 (s, 1H), 6.80 (s, 1H), 4.53 (s, 1H), 4.13 (d, J = 11.3 Hz, 1H), 3.95 (s, 1H), 3.76 (d, J = 22.3 Hz, 3H), 3.65 (d, J = 11.0 Hz, 1H), 3.49 (s, 1H), 3.18 (s, 1H), 2.62 (d, J = 9.7 Hz, 2H), 1.74 (s, 6H), 1.19 (d, J = 6.5 Hz, 3H) |
| 154 | 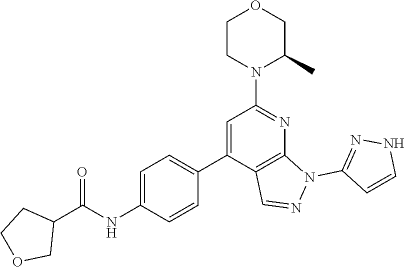 | 474.40 | ¹H NMR (400 MHz, DMSO-d6) δ 12.85 (s, 1H), 10.29 (s, 1H), 8.21 (s, 1H), 7.84 (m, 5H), 6.91 (s, 1H), 6.83 (d, J = 2.1 Hz, 1H), 4.58 (dd, J = 13.0, 7.1 Hz, 1H), 4.15 (d, J = 13.4 Hz, 1H), 3.98 (m, 2H), 3.85-3.65 (m, 5H), 3.52 (m, 1H), 3.21 (m, 2H), 2.16-2.07 (m, 2H), 1.22 (d, J = 6.6 Hz, 3H) |
| 155 | 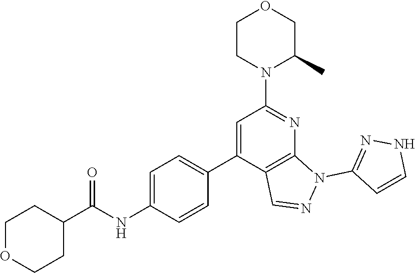 | 488.40 | ¹H NMR (400 MHz, DMSO-d6)δ 12.81 (s, 1H), 10.11 (s, 1H), 8.16 (s, 1H), 7.81 (d, J = 13.0 Hz, 5H), 6.83 (d, J = 25.0 Hz, 2H), 4.53 (s, 1H), 4.11 (d, J = 13.1 Hz, 1H), 3.93 (dd, J = 23.1, 10.9 Hz, 3H), 3.75 (d, J = 11.6 Hz, 1H), 3.64 (d, J = 10.5 Hz, 1H), 3.49 (t, J = 11.3 Hz, 1H), 3.37 (s, 2H), 3.22-3.15 (m, 1H), 2.66-2.56 (m, 1H), 1.68 (q, J = 10.7 Hz, 4H), 1.19 (d, J = 6.4 Hz, 3H) |
| 156 | 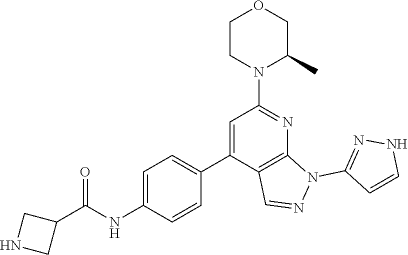 | 459.40 | ¹H NMR (400 MHz, DMSO-d6) δ 10.19 (s, 1H), 8.34 (s, 1H), 8.17 (s, 1H), 7.84-7.77 (m, 5H), 6.87 (s, 1H), 6.79 (s, 1H), 4.53 (s, 1H), 4.04 (dd, J = 64.4, 10.8 Hz, 1H), 3.87 (s, 1H), 3.70 (dt, J = 27.6, 12.5 Hz, 5H), 3.52 (s, 1H), 3.18 (s, 2H), 3.14 (s, 2H), 1.19 (d, J = 6.7 Hz, 3H) |
| 157 | 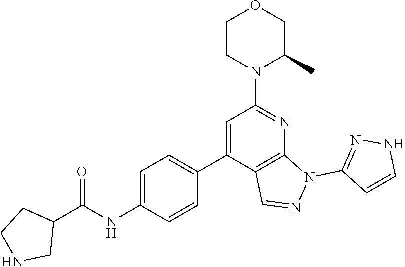 | 473.23 | ¹H NMR (400 MHz, DMSO-d6) δ 12.83 (s, 1H), 10.29 (d, J = 21.4 Hz, 1H), 8.21 (s, 1H), 7.83 (t, J = 6.3 Hz, 5H), 6.90 (s, 1H), 6.83 (d, J = 1.9 Hz, 1H), 4.58 (d, J = 6.0 Hz, 1H), 4.15 (d, J = 12.9 Hz, 1H), 3.99 (d, J = 8.2 Hz, 1H), 3.78 (d, J = 11.3 Hz, 1H), 3.68 (d, J = 9.0 Hz, 1H), 3.59- 3.48 (m, 2H), 3.26-3.16 (m, 2H), 2.96 (dd, J = 27.1, 16.7 Hz, 3H), 2.01 (dd, J = 24.5, 10.3 Hz, 2H), 1.89 (s, 1H), 1.22 (d, J = 6.6 Hz, 3H) |
| 158 | 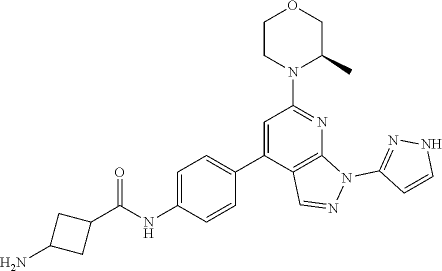 | 473.45 | ¹H NMR (400 MHz, DMSO-d6) δ 10.17 (s, 1H), 8.34 (s, 1H), 8.14 (s, 1H), 7.78 (s, 5H), 6.81 (d, J = 14.4 Hz, 2H), 4.49 (s, 1H), 4.08 (d, J = 13.7 Hz, 1H), 3.93 (s, 1H), 3.76 (s, 2H), 3.46 (d, J = 6.0 Hz, 3H), 3.15 (d, J = 15.9 Hz, 2H), 2.97 (s, 1H), 2.45-2.37 (m, 2H), 2.30-2.17 (m, 2H), 1.17 (d, J = 6.3 Hz, 3H) |
| 159 | 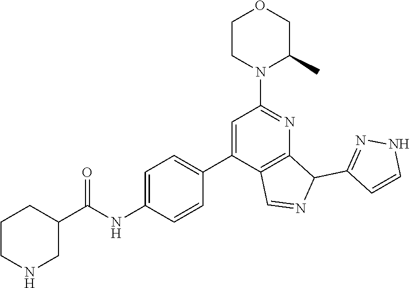 [SMILES: C[C@@H]1COCCN1c1cc(-c2ccc(NC(=O)C3CCCNC3)cc2)c2c(n1)C(c1cc[nH]n1)N=C2] | 487.35 | ¹H NMR (400 MHz, DMSO-d6) δ 12.86 (s, 1H), 10.49 (s, 1H), 8.78 (s, 1H), 8.16 (s, 1H), 7.84- 7.77 (m, 5H), 6.87 (s, 1H), 6.80 (s, 1H), 4.54 (s, 1H), 4.11 (d, J = 12.7 Hz, 1H), 3.96 (d, J = 11.7 Hz, 1H), 3.75 (d, J = 11.8 Hz, 1H), 3.64 (d, J = 10.7 Hz, 1H), 3.49 (s, 2H), 3.19 (d, J = 12.4 Hz, 2H), 3.02 (d, J = 12.3 Hz, 1H), 2.87 (s, 2H), 2.04 (s, 1H), 1.95 (s, 1H), 1.81 (s, 1H), 1.65 (d, J = 9.1 Hz, 2H), 1.19 (s, 3H) |
| 160 | 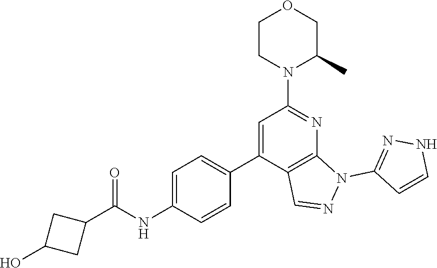 | 474.2 | ¹H NMR (400 MHz, DMSO-d6) δ 12.84 (s, 1H), 10.08 (s, 1H), 8.20 (s, 1H), 7.96-7.74 (m, 5H), 6.90 (s, 1H), 6.83 (s, 1H), 5.20 (d, J = 6.7 Hz, 1H), 4.57 (d, J = 5.2 Hz, 1H), 4.15 (d, J = 12.8 Hz, 1H), 3.99 (d, J = 7.9 Hz, 2H), 3.78 (d, J = 11.3 Hz, 1H), 3.67 (dd, J = 11.2, 2.5 Hz, 1H), 3.52 (dd, J = 11.9, 9.1 Hz, 1H), 3.21 (td, J = 12.8, 3.5 Hz, 1H), 2.73-2.58 (m, 1H), 2.38 (dt, J = 15.0, 5.1 Hz, 2H), 2.06 (td, J = 10.8, 2.6 Hz, 2H), 1.22 (d, J = 6.6 Hz, 3H) |
| 161 | 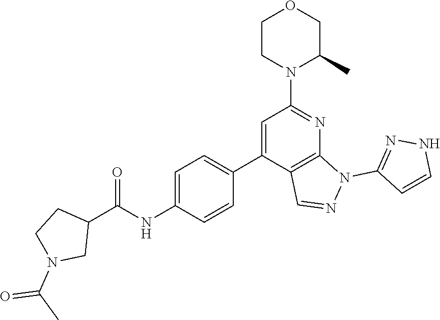 | 515.41 | ¹H NMR (400 MHz, DMSO-d6) δ 12.86 (s, 1H), 10.34 (d, J = 10.5 Hz, 1H), 8.21 (s, 1H), 7.91- 7.79 (m, 5H), 6.91 (s, 1H), 6.83 (d, J = 2.1 Hz, 1H), 4.58 (dd, J = 13.6, 7.3 Hz, 1H), 4.15 (d, J = 13.2 Hz, 1H), 3.99 (m, 1H), 3.81-3.58 (m, 4H), 3.53-3.44 (m, 2H), 3.24 (m, 3H), 2.14 (m, 2H), 1.97 (d, J = 5.1 Hz, 3H), 1.22 (d, J = 6.6 Hz, 3H) |
| 162 | 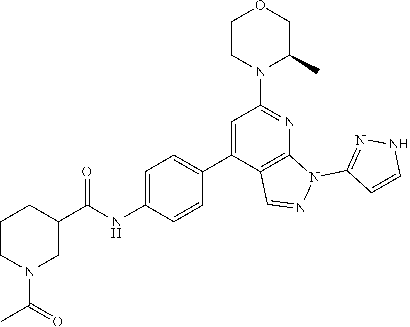 | 529.50 | ¹H NMR (400 MHz, DMSO-d6) δ 10.21 (d, J = 6.1 Hz, 1H), 8.17 (s, 1H), 7.85-7.76 (m, 5H), 6.87 (s, 1H), 6.80 (s, 1H), 4.53 (s, 1H), 4.43 (s, 1H), 4.12 (d, J = 11.9 Hz, 1H), 3.96 (d, J = 11.0 Hz, 1H), 3.88 (d, J = 13.5 Hz, 1H), 3.75 (d, J = 11.3 Hz, 2H), 3.66 (s, 1H), 3.19 (d, J = 12.4 Hz, 2H), 3.01 (t, J = 12.1 Hz, 1H), 2.66 (dd, J = 25.7, 12.6 Hz, 1H), 2.43-2.37 (m, 1H), 2.01 (d, J = 11.7 Hz, 3H), 1.98-1.93 (m, 1H), 1.76- 1.64 (m, 2H), 1.37 (d, J = 45.1 Hz, 1H), 1.19 (d, J = 6.5 Hz, 3H) |
| 163 | 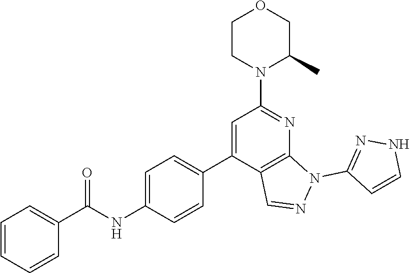 | 480.15 | ¹H NMR (400 MHz, CD₃OD) δ 8.13 (s, 1H), 7.97 (s, 5H), 7.95 (s, 1H), 7.94 (s, 1H), 7.92 (s, 1H), 7.82 (s, 2H), 7.81 (s, 1H), 7.76 (s, 1H), 7.59 (d, J = 6.4 Hz, 1H), 7.54 (d, J = 7.0 Hz, 2H), 6.99 (s, 1H), 6.86 (s, 1H), 4.60 (s, 1H), 4.17 (d, J = 13.5 Hz, 2H), 4.04 (d, J = 11.4 Hz, 1H), 3.82 (d, J = 8.0 Hz, 2H), 3.65 (s, 1H), 1.32 (s, 3H) |
| 164 | 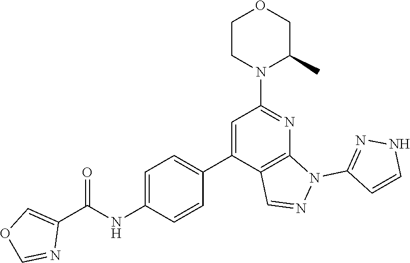 | 471.20 | ¹H NMR (400 MHz, DMSO-d6) δ 12.82 (s, 1H), 10.41 (s, 1H), 8.88-8.82 (m, 1H), 8.66-8.62 (m, 1H), 8.21-8.16 (m, 1H), 8.05-8.01 (m, 2H), 7.87-7.83 (m, 3H), 6.92-6.88 (m, 1H), 6.83-6.78 (m, 1H), 4.56 (s, 1H), 4.13 (d, J = 12.2 Hz, 1H), 3.96 (d, J = 10.8 Hz, 1H), 3.78- 3.72 (m, 1H), 3.68-3.63 (m, 1H), 3.50 (t, J = 11.3 Hz, 1H), 3.20 (d, J = 12.3 Hz, 1H), 1.21- 1.18 (m, 3H) |
| 165 | 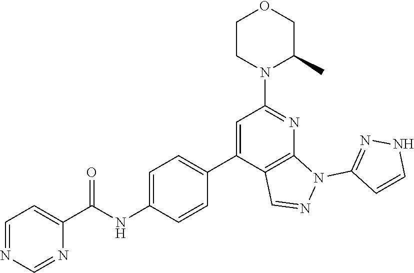 | 482.40 | ¹H NMR (400 MHz, CD₃OD) δ 9.37 (s, 1H), 9.09 (d, J = 5.3 Hz, 1H), 8.22 (d, J = 4.7 Hz, 1H), 8.14 (s, 1H), 8.05 (d, J = 7.6 Hz, 2H), 7.86 (d, J = 7.9 Hz, 2H), 7.76 (s, 1H), 6.99 (s, 1H), 6.88 (s, 1H), 4.61 (d, J = 8.2 Hz, 2H), 4.18 (d, J = 12.6 Hz, 1H), 4.04 (d, J = 12.5 Hz, 1H), 3.88-3.76 (m, 2H), 3.65 (t, J = 11.8 Hz, 1H), 1.33 (d, J = 6.5 Hz, 3H) |
| 166 | 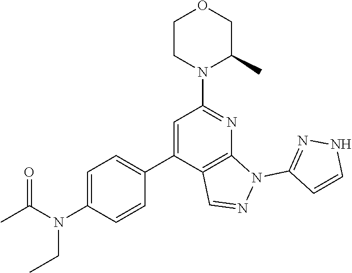 | 446.22 | |
| 167 | 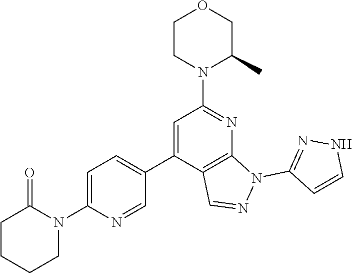 | 459.22 | ¹H NMR (500 MHz, DMSO-d6) δ 12.86 (s, 1H), 8.94 (d, J = 2.2 Hz, 1H), 8.29 (dd, J = 8.6, 2.5 Hz, 1H), 8.24 (s, 1H), 7.96 (d, J = 8.6 Hz, 1H), 7.87 (s, 1H), 7.03 (s, 1H), 6.83 (d, J = 2.1 Hz, 1H), 4.60 (d, J = 5.1 Hz, 1H), 4.18 (d, J = 12.7 Hz, 1H), 4.00 (dd, J = 11.2, 3.2 Hz, 1H), 3.96 (t, J = 5.9 Hz, 2H), 3.79 (d, J = 11.3 Hz, 1H), 3.68 (dd, J = 11.4, 2.7 Hz, 1H), 3.53 (td, J = 11.8, 2.7 Hz, 1H), 3.23 (td, J = 12.8, 3.5 Hz, 1H), 2.54 (t, J = 6.7 Hz, 2H), 1.98-1.79 (m, 4H), 1.23 (d, J = 6.7 Hz, 3H) |
| 168 | 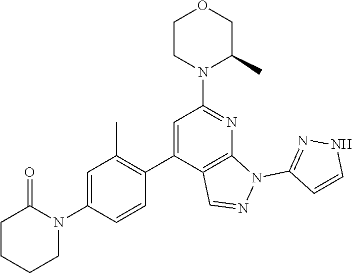 | 472.30 | ¹H NMR (400 MHz, DMSO-d6) δ 12.82 (s, 1H), 7.84 (s, 1H), 7.72 (s, 1H), 7.35 (d, J = 7.9 Hz, 1H), 7.29 (s, 1H), 7.22 (d, J = 7.5 Hz, 1H), 6.80 (s, 1H), 6.69 (s, 1H), 4.46 (s, 1H), 4.10 (d, J = 13.3 Hz, 1H), 3.94 (d, J = 12.4 Hz, 1H), 3.71 (d, J = 11.2 Hz, 1H), 3.64 (s, 3H), 3.48 (s, 1H), 3.15 (s, 1H), 2.39 (s, 2H), 2.22 (s, 3H), 1.85 (s, 4H), 1.18 (d, J = 6.6 Hz, 3H) |
| 169 | 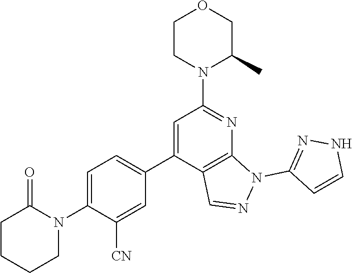 | 482.32 | ¹H NMR (400 MHz, dmso) δ 8.46 (s, 1H), 8.38 (d, J = 1.9 Hz, 1H), 8.27-8.20 (m, 2H), 7.85 (s, 1H), 7.70 (d, J = 8.3 Hz, 1H), 7.04 (s, 1H), 6.80 (s, 1H), 4.58 (d, J = 5.1 Hz, 1H), 4.18 (d, J = 12.7 Hz, 1H), 3.97 (d, J = 10.8 Hz, 1H), 3.76 (d, J = 11.3 Hz, 1H), 3.66 (s, 3H), 3.50 (t, J = 10.8 Hz, 1H), 3.23 - 3.15 (m, 2H), 2.44 (s, 1H), 1.95- 1.86 (m, 4H), 1.21 (d, J = 6.3 Hz, 3H) |
| 170 | 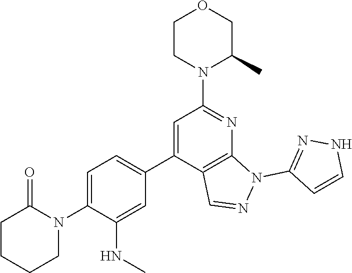 | 487.25 | |
| 171 | 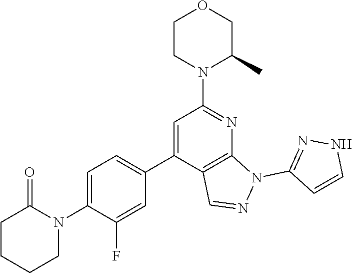 | 476.35 | ¹H NMR (400 MHz, DMSO-d6) δ 12.83 (s, 1H), 8.17 (s, 1H), 7.85-7.71 (m, 3H), 7.54 (t, J = 8.1 Hz, 1H), 6.95 (s, 1H), 6.79 (s, 1H), 4.56 (s, 1H), 4.15 (d, J = 11.7 Hz, 1H), 3.96 (d, J = 10.8 Hz, 1H), 3.74 (d, J = 11.5 Hz, 1H), 3.64 (d, J = 11.6 Hz, 1H), 3.59 (s, 2H), 3.49 (t, J = 11.6 Hz, 1H), 3.17 (d, J = 11.4 Hz, 1H), 2.41 (s, 2H), 1.87 (s, 4H), 1.19 (d, J = 5.9 Hz, 3H) |
| 172 | 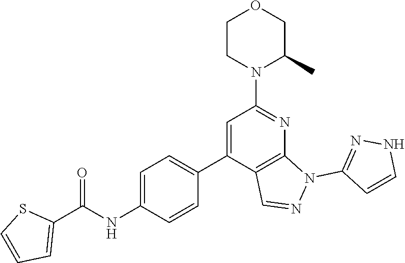 | 486.29 | ¹H NMR (400 MHz, DMSO-d6) δ 10.47 (s, 1H), 8.24 (s, 1H), 8.10 (d, J = 2.9 Hz, 1H), 7.97 (d, J = 8.7 Hz, 2H), 7.91 (dd, J = 6.4, 1.1 Hz, 3H), 7.85 (d, J = 2.1 Hz, 1H), 7.27 (dd, J = 4.9, 3.9 Hz, 1H), 6.94 (s, 1H), 6.84 (d, J = 2.2 Hz, 1H), 6.09 (s, 1H), 4.59 (d, J = 4.5 Hz, 1H), 4.17 (d, J = 12.6 Hz, 1H), 4.00 (dd, J = 11.4, 3.5 Hz, 1H), 3.79 (d, J = 11.2 Hz, 1H), 3.69 (dd, J = 11.3, 2.6 Hz, 1H), 3.60-3.49 (m, 1H), 3.27-3.15 (m, 1H), 1.24 (d, J = 6.6 Hz, 3H) |
| 173 | 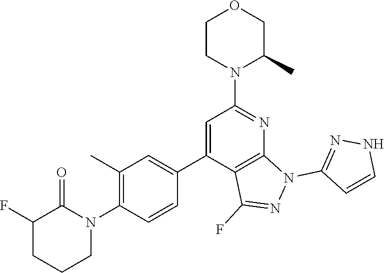 | 508.22 | |
| 174 | 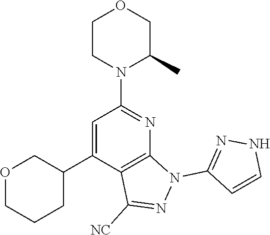 | 394.19 | |
| 175 | 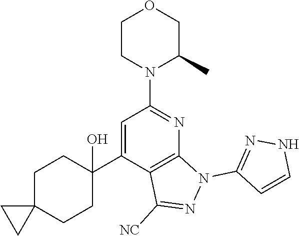 | 434.22 | |
| 176 | 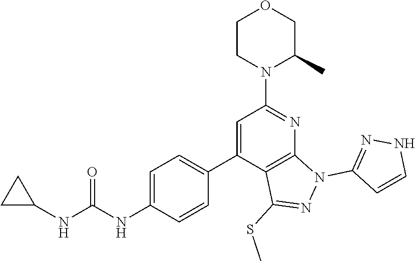 | 505.21 | |
| 177 | 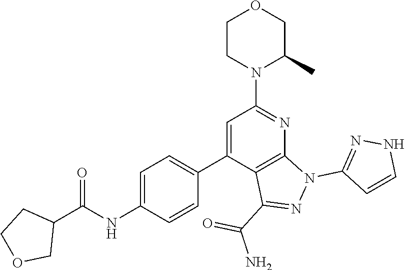 | 517.22 | |
| 178 | 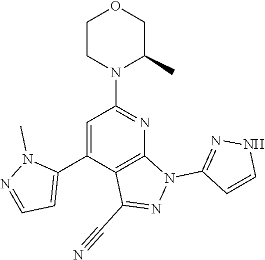 | 390.17 | |
| 179 | 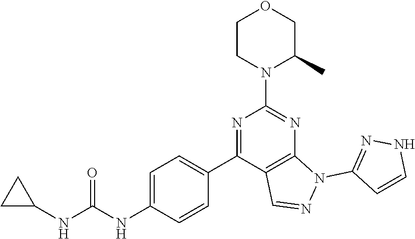 | 460.21 | |
| 180 | 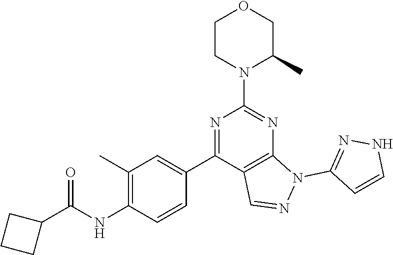 | 473.23 | |
| 181 | 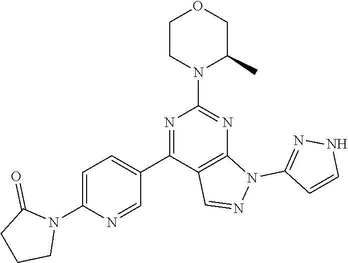 | 446.20 | |
| 182 | 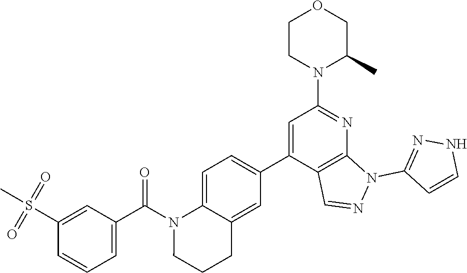 | 598.22 | |
| 183 | 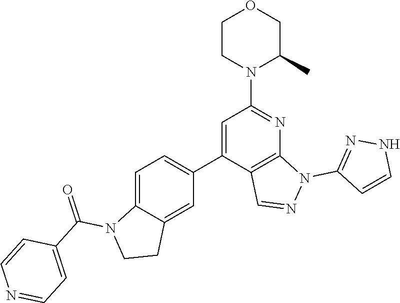 | 507.40 | ¹H NMR (400 MHz, DMSO-d6) δ 8.73 (d, J = 4.2 Hz, 2H), 8.21 (d, J = 22.8 Hz, 2H), 7.82 (d, J = 18.7 Hz, 3H), 7.60 (s, 2H), 6.84 (d, J = 32.7 Hz, 1H), 4.55 (s, 1H), 4.13-3.95 (m, 2H), 3.75 (d, J = 10.8 Hz, 1H), 3.65 (d, J = 9.4 Hz, 0H), 3.49 (s, 1H), 3.20 (s, 2H), 1.20 (s, 2H) |
| 184 | 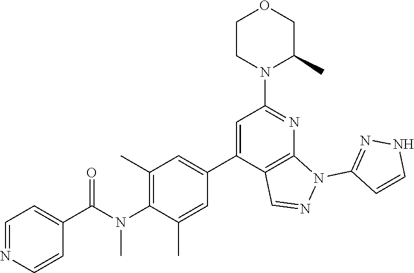 | 523.45 | ¹H NMR (400 MHz, CD₃OD) δ 8.44 (d, J = 6.1 Hz, 2H), 8.01 (s, 1H), 7.75 (s, 1H), 7.49 (s, 2H), 7.32 (d, J = 6.1 Hz, 2H), 6.96 (s, 1H), 6.78 (s, 1H), 4.62 (s, 1H), 4.57 (t, J = 6.5 Hz, 1H), 4.14 (d, J = 14.4 Hz, 1H), 4.02 (d, J = 7.6 Hz, 1H), 3.82 (d, J = 11.4 Hz, 1H), 3.77 (d, J = 11.2 Hz, 1H), 3.62 (t, J = 11.7 Hz, 1H), 3.39 (s, 3H), 2.37 (s, 6H), 1.30 (d, J = 6.7 Hz, 3H) |
| 185 | 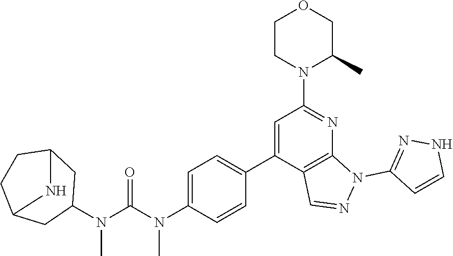 | 556.31 | |
| 186 | 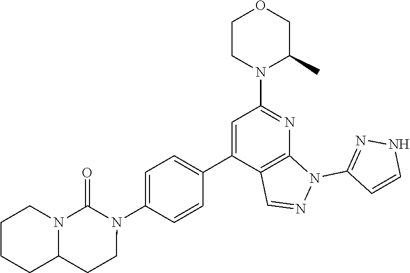 | 513.26 | |
| 187 | 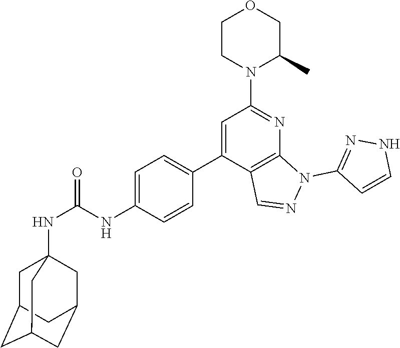 | 553.40 | ¹H NMR (400 MHz, DMSO-d6) δ 8.54 (s, 1H), 8.18 (s, 1H), 7.84 (s, 1H), 7.75 (d, J = 8.6 Hz, 2H), 7.54 (d, J = 8.7 Hz, 2H), 6.89-6.77 (m, 2H), 5.97 (s, 1H), 4.56 (dd, J = 10.2, 4.5 Hz, 1H), 4.14 (d, J = 12.7 Hz, 1H), 3.98 (d, J = 10.9 Hz, 1H), 3.78 (d, J = 11.3 Hz, 1H), 3.67 (d, J = 11.2 Hz, 1H), 3.52 (t, J = 11.1 Hz, 1H), 3.21 (t, J = 12.6 Hz, 1H), 2.05 (s, 3H), 1.96 (s, 6H), 1.65 (s, 6H), 1.22 (d, J = 6.7 Hz, 3H) |
| 188 | 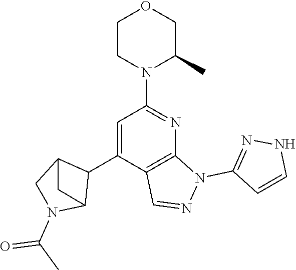 | 408.21 | |
| 189 | 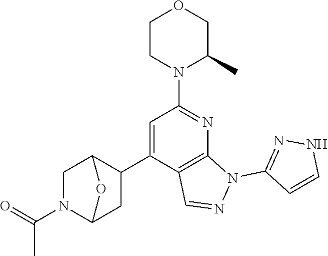 | 424.20 | |
| 190 | 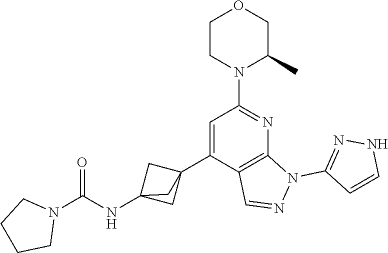 | 463.25 | |
| 191 | 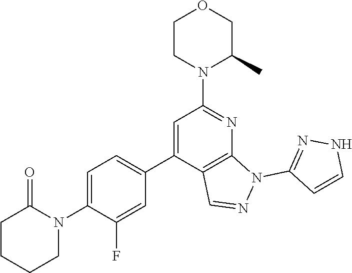 | 476.35 | ¹H NMR (400 MHz, DMSO-d6) δ 12.83 (s, 1H), 8.17 (s, 1H), 7.85-7.71 (m, 3H), 7.54 (t, J = 8.1 Hz, 1H), 6.95 (s, 1H), 6.79 (s, 1H), 4.56 (s, 1H), 4.15 (d, J = 11.7 Hz, 1H), 3.96 (d, J = 10.8 Hz, 1H), 3.74 (d, J = 11.5 Hz, 1H), 3.64 (d, J = 11.6 Hz, 1H), 3.59 (s, 2H), 3.49 (t, J = 11.6 Hz, 1H), 3.17 (d, J = 11.4 Hz, 1H), 2.41 (s, 2H), 1.87 (s, 4H), 1.19 (d, J = 5.9 Hz, 3H) |
| 192 | 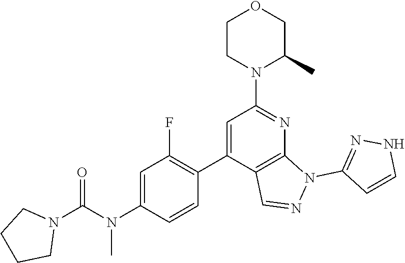 | 505.50 | ¹H NMR (400 MHz, CD₃OD) δ 7.91 (s, 1H), 7.75 (s, 1H), 7.65 (s, 1H), 7.12 (d, J = 9.2 Hz, 2H), 6.96 (s, 1H), 6.83 (s, 1H), 4.53 (s, 1H), 4.14 (d, J = 13.4 Hz, 1H), 4.02 (d, J = 10.0 Hz, 1H), 3.80 (d, J = 8.5 Hz, 2H), 3.63 (s, 1H), 3.33 (s, 1H), 3.27 (s, 3H), 3.21 (s, 4H), 1.80 (s, 4H), 1.31 (d, J = 6.1 Hz, 4H) |
| 193 | 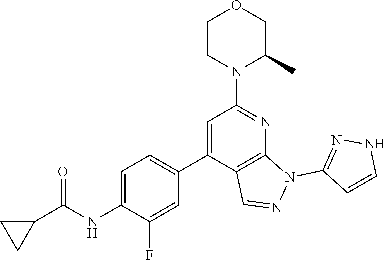 | 462.20 | |
| 194 | 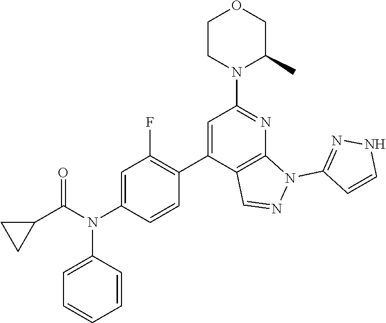 | 538.23 | |
| 195 | 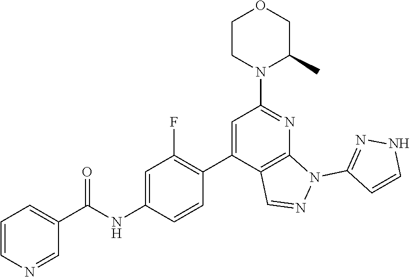 | 499.19 | ¹H NMR (400 MHz, DMSO-d6) δ 12.86 (s, 1H), 10.84 (s, 1H), 9.16 (s, 1H), 8.81 (d, J = 4.3 Hz, 1H), 8.35 (dt, J = 8.0, 1.8 Hz, 1H), 7.99 (d, J = 12.6 Hz, 2H), 7.87 (s, 1H), 7.77 (d, J = 4.6 Hz, 2H), 7.62 (dd, J = 7.9, 4.9 Hz, 1H), 6.90 (s, 1H), 6.83 (s, 1H), 4.53 (d, J = 5.8 Hz, 1H), 4.13 (d, J = 12.7 Hz, 1H), 3.99 (dd, J = 11.0, 2.8 Hz, 1H), 3.78 (d, J = 11.2 Hz, 1H), 3.68 (dd, J = 11.3, 2.5 Hz, 1H), 3.53 (td, J = 11.7, 2.6 Hz, 1H), 3.22 (td, J = 13.0, 3.5 Hz, 1H), 1.24 (d, J = 6.7 Hz, 3H) |
| 196 | 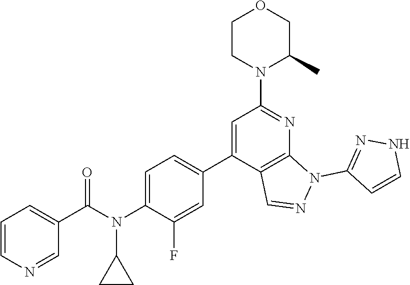 | 539.22 | |
| 197 | 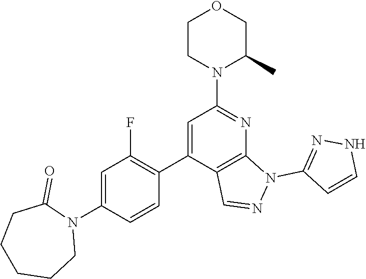 | 490.23 | ¹H NMR (400 MHz, DMSO-d6) δ 12.87 (s, 1H), 7.93 (s, 1H), 7.85 (s, 1H), 7.72 (t, J = 8.4 Hz, 1H), 7.37 (dd, J = 12.0, 2.0 Hz, 1H), 7.28 (dd, J = 8.3, 2.0 Hz, 1H), 6.89 (s, 1H), 6.82 (d, J = 2.1 Hz, 1H), 4.51 (d, J = 6.2 Hz, 1H), 4.13 (d, J = 12.4 Hz, 1H), 4.03-3.94 (m, 1H), 3.85 (s, 2H), 3.77 (d, J = 11.4 Hz, 1H), 3.67 (dd, J = 11.3, 2.7 Hz, 1H), 3.56-3.47 (m, 1H), 3.21 (td, J = 12.9, 3.7 Hz, 1H), 2.71-2.62 (m, 2H), 1.76 (d, J = 8.8 Hz, 6H), 1.23 (d, J = 6.7 Hz, 3H) |
| 198 | 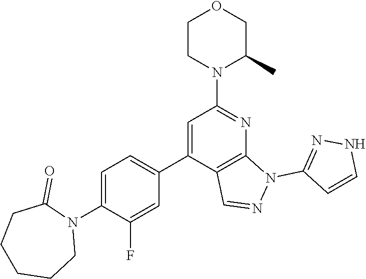 | 490.23 | |
| 199 | 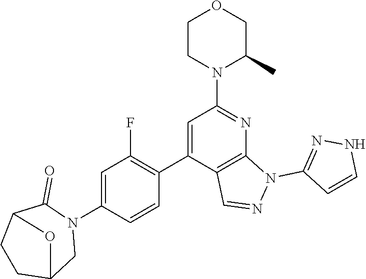 | 504.29 | ¹H NMR (400 MHz, DMSO-d6) δ 12.87 (s, 1H), 7.93 (s, 1H), 7.86 (s, 1H), 7.75 (t, J = 8.5 Hz, 1H), 7.61 (d, J = 12.3 Hz, 1H), 7.47 (dd, J = 8.4, 1.9 Hz, 1H), 6.90 (s, 1H), 6.82 (d, J = 1.8 Hz, 1H), 4.84-4.75 (m, 1H), 4.61-4.47 (m, 2H), 4.06 (ddd, J = 24.6, 21.0, 7.8 Hz, 3H), 3.77 (d, J = 11.3 Hz, 1H), 3.67 (dd, J = 11.4, 2.6 Hz, 1H), 3.56-3.46 (m, 2H), 3.25-3.16 (m, 1H), 2.19- 2.01 (m, 4H), 1.23 (d, J = 6.6 Hz, 3H) |

### Example 200

### Preparation of 3-(2-fluoro-4-(6-((R)-3-methylmorpholino)-1-(1H-pyrazol-3-yl)-1H-pyrazolo[3,4-b]pyridin-4-yl)phenyl)-8-oxa-3-azabicyclo[3.2.1]octan-2-one

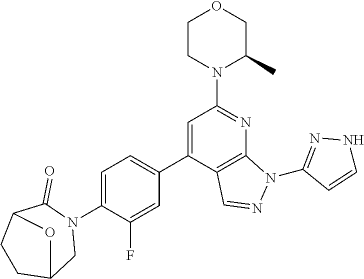 [SMILES: C[C@@H]1COCCN1c1cc(-c2ccc(N3CC4CCC(O4)C3=O)c(F)c2)c2cnn(-c3cc[nH]n3)c2n1]

### Step 1: Preparation of (R)-4-(4-(4-bromo-3-fluorophenyl)-1-(1-((2-(trimethylsilyl)ethoxy)methyl)-1H-pyrazol-3-yl)-1H-pyrazolo[3,4-b]pyridin-6-yl)-3-methylmorpholine

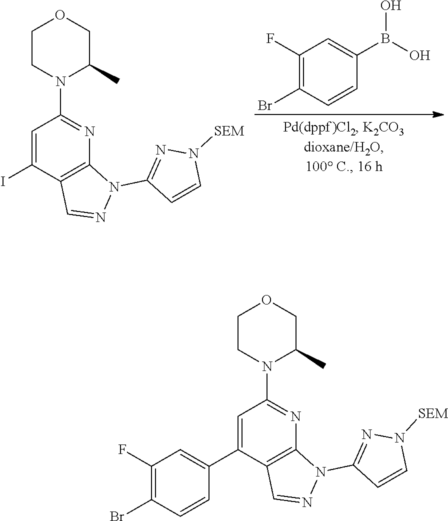

(R)-4-(4-Iodo-1-(1-((2-(trimethylsilyl)ethoxy)methyl)-1H-pyrazol-3-yl)-1H-pyrazolo[3,4-b]pyridin-6-yl)-3-methylmorpholine (150 mg, 0.28 mmol, 1.0 equiv.), (4-bromo-3-fluorophenyl)boronic acid (60.7 mg, 0.28 mmol, 1.0 equiv.), Pd(dppf)Cl₂ (41 mg, 0.55 mmol, 0.2 equiv.) and K₂CO₃ (76 mg. 5.5 mmol, 2 equiv.) were dissolved in dioxane (5 mL) and water (0.5 mL), and the mixture was reacted at 100° C. under nitrogen protection for 16 h. After the reaction was completed as monitored by TLC, the reaction liquid was cooled to room temperature. The solid was filtered, and the filtrate was spun to dryness, to afford the crude product. The crude product was purified by column chromatography (petroleum ether:ethyl acetate=4:1), to afford the target compound (160 mg). LCMS (ESI) [M+H]⁺=587.27.

### Step 2: Preparation of 3-(2-fluoro-4-(6-((R)-3-methylmorpholino)-1-(1-((2-(trimethylsilyl)ethoxy)methyl)-1H-pyrazol-3-yl)-1H-pyrazolo[3,4-b]pyridin-4-yl)phenyl)-8-oxa-3-azabicyclo[3.2.1]octan-2-one

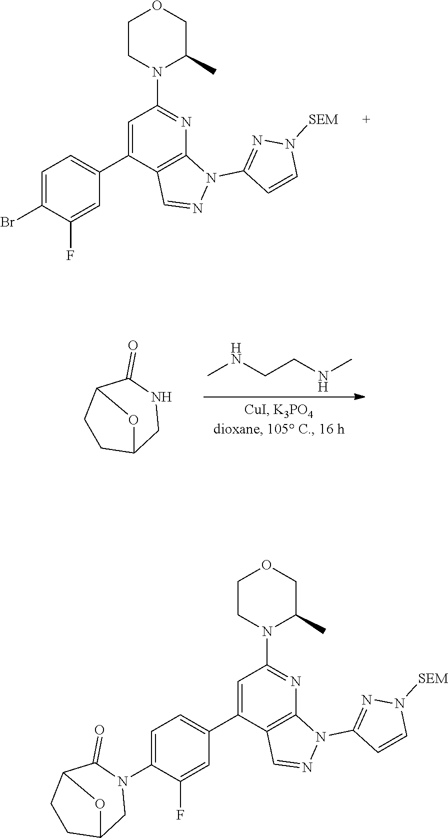

(R)-4-(4-(4-Bromo-3-fluorophenyl)-1-(1-((2-(trimethylsilyl)ethoxy)methyl)-1H-pyrazol-3-yl)-1H-pyrazole[3,4-b]pyridin-6-yl)-3-methylmorpholine (160 mg, 0.27 mmol, 1.0 equiv.), 8-oxa-3-azabicyclo[3.2.1]octan-2-one (35 mg, 0.27 mmol, 1.0 equiv.), N¹,N²-dimethylethane-1,2-diamine (9.5 mg, 0.1 mmol, 0.4 equiv.), cuprous iodide (19 mg, 0.1 mmol, 0.4 equiv.) and potassium phosphate (114 mg, 0.54 mmol, 2.0 equiv.) were dissolved in anhydrous dioxane (5 mL), and the mixture was reacted at 105° C. under nitrogen protection for 16 hours. After the reaction was completed as monitored by LCMS, the reaction system was cooled to room temperature. The solid was filtered, and the filtrate was spun to dryness, to afford the crude product. The crude product was purified by column chromatography (petroleum ether:ethyl acetate=3:1), to afford the target compound (120 mg, yield: 70.21%). LCMS (ESI) [M+H]⁺=634.49.

### Step 3: Preparation of 3-(2-fluoro-4-(6-((R)-3-methylmorpholino)-1-(1H-pyrazol-3-yl)-1H-pyrazolo[3,4-b]pyridin-4-yl)phenyl)-8-oxa-3-azabicyclo[3.2.1]octan-2-one

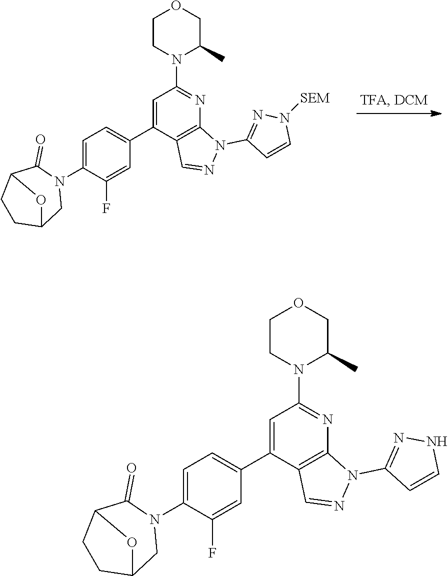

3-(2-Fluoro-4-(6-((R)-3-methylmorpholino)-1-(1-((2-(trimethylsilyl)ethoxy)methyl)-1H-pyrazol-3-yl)-1H-pyrazolo[3,4-b]pyridin-4-yl)phenyl)-8-oxa-3-azabicyclo[3.2.1]octan-2-one (120 mg, 0.19 mmol) was dissolved in dichloromethane (5 mL), trifluoroacetic acid (2 mL) was added, and the mixture was stirred at room temperature for 4 hours. After the reaction was completed as monitored by LCMS, the reaction liquid was spun to dryness to remove the solvent. The reaction was quenched with sodium bicarbonate aqueous solution (10 mL), the reaction liquid was extracted with ethyl acetate (3×30 mL), and the organic phases were combined, washed with saturated sodium chloride aqueous solution (3×30 mL), dried over anhydrous sodium sulfate, filtered and concentrated, to afford the crude product. The crude product was purified by reverse-phase column chromatography (C18, water:acetonitrile=1:1), to afford the target compound (35 mg, yield: 36.81%). LCMS (ESI) [M+H]⁺=504.29; ¹H NMR (400 MHz, DMSO-d6) δ 12.87 (s, 1H), 8.21 (s, 1H), 7.84 (dd, J=11.2, 1.7 Hz, 2H), 7.77 (dd, J=8.2, 1.6 Hz, 1H), 7.64 (t, J=8.0 Hz, 1H), 6.99 (s, 1H), 6.82 (d, J=2.0 Hz, 1H), 4.78 (dd, J=7.1, 4.2 Hz, 1H), 4.60 (d, J=4.6 Hz, 1H), 4.56 (d, J=5.7 Hz, 1H), 4.19 (d, J=12.4 Hz, 1H), 4.03-3.92 (m, 2H), 3.78 (d, J=11.3 Hz, 1H), 3.67 (dd, J=11.3, 2.7 Hz, 1H), 3.52 (td, J=11.8, 2.8 Hz, 1H), 3.39 (d, J=11.1 Hz, 1H), 3.22 (td, J=13.0, 3.7 Hz, 1H), 2.21-2.03 (m, 4H), 1.23 (d, J=6.7 Hz, 3H).

### Example 201

### Preparation of (3R)-4-(4-(4-ethylcyclohex-1-en-1-yl)-1-(1H-pyrazol-5-yl)-1H-pyrazole[3,4-b]pyridin-6-yl)-3-methylmorpholine

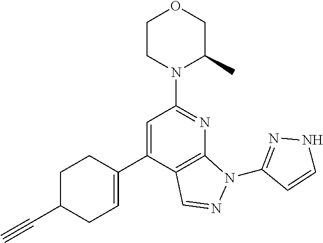 [SMILES: C#CC1CC=C(c2cc(N3CCOC[C@H]3C)nc3c2cnn3-c2cc[nH]n2)CC1]

The target compound (16 mg, yield: 37.48%) was afforded with reference to the preparation method of Example 147. LCMS (ESI) [M+H]⁺=389.30; ¹H NMR (400 MHz, DMSO-d6) δ12.81 (s, 1H), 8.15 (s, 1H), 7.81 (s, 1H), 6.79 (d, J=2.2 Hz, 1H), 6.66 (s, 1H), 6.45 (s, 1H), 4.49 (d, J=5.1 Hz, 1H), 4.06 (d, J=13.0 Hz, 1H), 3.96 (dd, J=11.3, 3.1 Hz, 1H), 3.75 (d, J=11.3 Hz, 1H), 3.65 (dd, J=11.3, 2.6 Hz, 1H), 3.55-3.44 (m, 1H), 3.16 (td, J=12.7, 3.6 Hz, 1H), 2.95 (dd, J=2.2, 1.6 Hz, 1H), 2.73 (s, 1H), 2.59 (s, 3H), 2.34 (s, 1H), 2.30 (d, J=8.4 Hz, 1H), 2.03 (d, J=13.0 Hz, 1H), 1.85-1.72 (m, 1H), 1.18 (d, J=6.6 Hz, 3H).

### Example 202

### Preparation of (R)-4-(4-(4-ethynylcyclohexyl)-1-(1H-pyrazol-5-yl)-1H-pyrazolo[3,4-b]pyridin-6-yl)-3-methylmorpholine

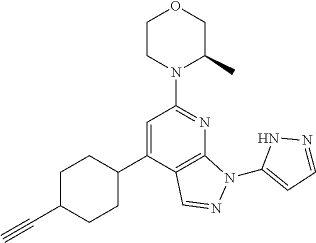 [SMILES: C#CC1CCC(c2cc(N3CCOC[C@H]3C)nc3c2cnn3-c2ccn[nH]2)CC1]

### Step 1: Preparation of (R)-(4-(6-(3-methylmorpholino)-1-(1-((2-(trimethylsilyl)ethoxy)methyl)-1H-pyrazol-5-yl)-1H-pyrazolo[3,4-)b]pyridin-4-yl)cyclohexyl)methanol

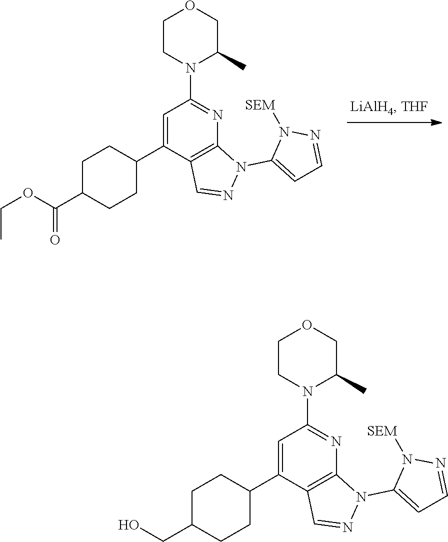

Ethyl (R)-4-(6-(3-methylmorpholinyl)-1-(1-((2-(trimethylsilyl)ethoxy)methyl)-1H-pyrazol-5-yl)-1H-pyrazole[3,4-b]pyridin-4-yl)cyclohexane-1-carboxylate (500 mg, 0.88 mmol, 1.0 equiv.) was dissolved in anhydrous tetrahydrofuran (10 mL), then lithium aluminum hydride (67 mg, 1.76 mmol, 2 equiv.) was added under ice bath, and the mixture was reacted for 1 hour under ice bath. After the reaction was completed, water was added, the solid was filtered, and the filtrate was extracted with ethyl acetate (3×30 mL). The organic phases were combined, washed with saturated sodium chloride aqueous solution (3×30 mL), dried over anhydrous sodium sulfate, filtered and concentrated, to afford the crude product (330 mg). LCMS (ESI) [M+H]⁺=527.34.

### Step 2: Preparation of (R)-4-(6-(3-methylmorpholinyl)-1-(1-((2-(trimethylsilyl)ethoxy)methyl)-1H-pyrazol-5-yl)-1H-pyrazole[3,4-b]pyridin-4-yl)cyclohexane-1-carbaldehyde

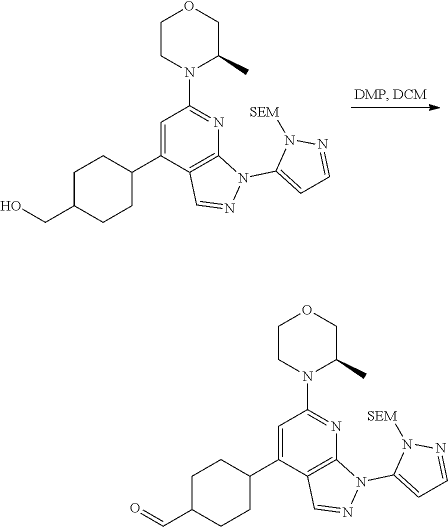

(R)-(4-(6-(3-Methylmorpholino)-1-(1-((2-(trimethylsilyl)ethoxy)methyl)-1H-pyrazol-5-yl)-1H-pyrazolo[3,4-)b]pyridin-4-yl)cyclohexyl)methanol (330 mg, 0.63 mmol, 1.0 equiv.) was dissolved in anhydrous dichloromethane (10 mL), Dess-Martin periodinane (534 mg, 1.26 mmol, 2 equiv.) was added under ice bath, and the mixture was reacted at room temperature for 2 hours. After the reaction was completed, the solid was filtered, and the filtrate was concentrated, to afford the crude product. The crude product was purified by column chromatography (petroleum ether:ethyl acetate=1:1), to afford the target compound (100 mg, yield: 30.29%). LCMS (ESI) [M+H]⁺=525.40.

### Step 3: Preparation of (R)-4-ethynyl-1-(6-(3-methylmorpholinyl)-1-(1-((2-(trimethylsilyl)ethoxy)methyl)-1H-pyrazol-5-yl)-1H-pyrazole[3,4-b]pyridin-4-yl)cyclohexane

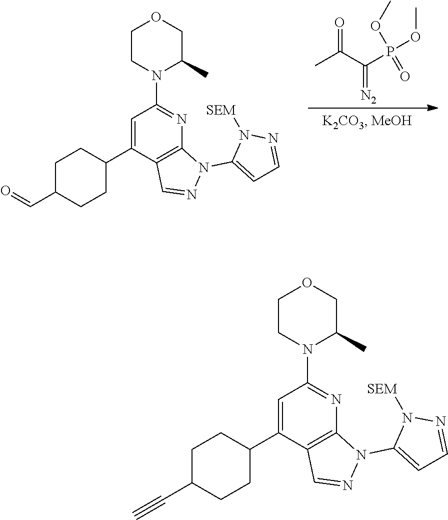

(R)-4-(6-(3-Methylmorpholinyl)-1-(1-((2-(trimethylsilyl)ethoxy)methyl)-1H-pyrazol-5-yl)-1H-pyrazole[3,4-b]pyridin-4-yl)cyclohexane-1-carbaldehyde (100 mg, 0.19 mmol, 1.0 equiv.) and potassium carbonate (79 mg, 0.57 mmol, 3 equiv.) were dissolved in methanol (5 mL), dimethyl (1-diazo-2-oxopropyl)phosphonate (54 mg, 0.28 mmol, 1.5 equiv.) was added under ice bath, and then the mixture was stirred under ice bath for 30 minutes and at room temperature for 4 hours. After the reaction was completed, the reaction liquid was diluted with water and extracted with ethyl acetate (3×30 mL), and the organic phases were combined, washed with saturated sodium chloride aqueous solution (3×30 mL), dried over anhydrous sodium sulfate, filtered and concentrated, to afford the crude product. The crude product was purified by column chromatography (petroleum ether:ethyl acetate=1:1), to afford the target compound (60 mg, yield: 60.49%). LCMS (ESI) [M+H]⁺=521.35.

### Step 4: Preparation of (R)-4-ethynyl-1-(6-(3-methylmorpholinyl)-1-(1H-pyrazolyl-5-yl)-1H-pyrazolyl[3,4-b]pyridin-4-yl)cyclohexane

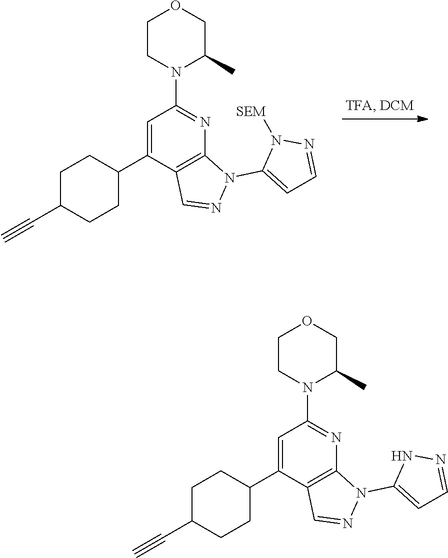

(R)-4-Ethynyl-1-(6-(3-methylmorpholinyl)-1-(1-((2-(trimethylsilyl)ethoxy)methyl)-1H-pyrazol-5-yl)-1H-pyrazole[3,4-b]pyridin-4-yl)cyclohexane (60 mg, 0.11 mmol) was dissolved in dichloromethane (5 mL), then trifluoroacetic acid (2 mL) was added, and the mixture was stirred at room temperature for 4 hours. After the reaction was completed as monitored by LCMS, the reaction liquid was spun to dryness to remove the solvent. Subsequently, the reaction was quenched with sodium bicarbonate aqueous solution (10 mL), the reaction liquid was extracted with ethyl acetate (3×30 mL), and the organic phases were combined, washed with saturated sodium chloride aqueous solution (3×30 mL), dried over anhydrous sodium sulfate, filtered and concentrated, to afford the crude product. The crude product was purified by reverse-phase column chromatography (C18, water:acetonitrile=1:1), to afford the target compound (2.4 mg, yield: 5.6%). LCMS (ESI) [M+H]⁺=391.32; ¹H NMR (400 MHz, DMSO-d6) δ 12.77 (s, 1H), 8.20 (s, 1H), 7.82 (s, 1H), 6.77 (s, 1H), 6.62 (s, 1H), 4.45 (s, 1H), 4.11-3.90 (m, 2H), 3.75 (d, J=11.3 Hz, 1H), 3.64 (d, J=9.1 Hz, 1H), 3.48 (t, J=10.6 Hz, 1H), 3.15 (t, J=11.2 Hz, 1H), 2.89 (t, J=9.5 Hz, 2H), 2.43-2.40 (m, 1H), 2.05 (d, J=11.0 Hz, 2H), 1.87 (d, J=10.8 Hz, 2H), 1.72 (dd, J=25.1, 12.4 Hz, 2H), 1.53 (dd, J=24.7, 12.2 Hz, 2H), 1.18 (d, J=6.6 Hz, 3H).

### Examples 203-269

The following compounds of Examples 203-269 were prepared with reference to the preparation methods of Examples 1-18, 30-33 and 147.

| Example | | LCMS (ESI) | |
|---|---|---|---|
| No. | Structure | [M + H]⁺ | ¹H NMR |
| 203 | 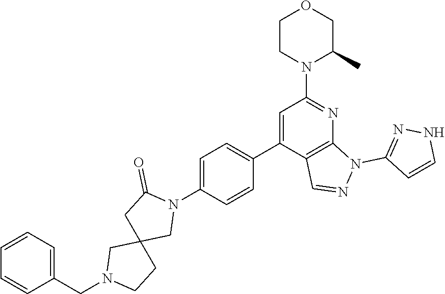 [SMILES: C[C@@H]1COCCN1c1cc(-c2ccc(N3CC4(CCN(Cc5ccccc5)C4)CC3=O)cc2)c2cnn(-c3cc[nH]n3)c2n1] | 589.50 | ¹H NMR (400 MHz, CD3OD) δ 8.10 (s, 1H), 7.84-7.74 (m, 5H), 7.35-7.29 (m, 7.1 Hz, 4H), 7.25 (d, J = 6.0 Hz, 1H), 6.98 (s, 1H), 6.84 (s, 1H), 4.59 (dd, J = 11.2, 4.9 Hz, 1H), 4.16 (d, J = 13.9 Hz, 1H), 4.03 (d, J = 9.6 Hz, 1H), 3.97-3.88 (m, 2H), 3.81 (q, J = 11.8 Hz, 2H), 3.69-3.60 (m, 3H), 3.36 (d, J = 12.0 Hz, 1H), 2.82-2.68 (m, 5H), 2.61 (d, J = 10.6 Hz, 1H), 2.07-1.96 (m, 2H), 1.32 (d, J = 6.7 Hz, 3H) |
| 204 | 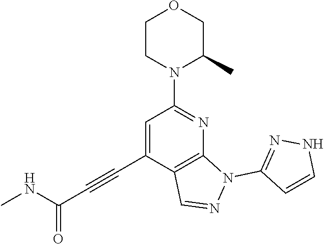 | 366.10 | ¹H NMR (400 MHz, CD3OD) δ 8.09 (s, 1H), 7.75 (s, 1H), 6.99 (s, 1H), 6.90 (s, 1H), 4.48 (s, 1H), 4.05 (dd, J = 29.4, 11.9 Hz, 2H), 3.78 (dd, J = 24.7, 11.5 Hz, 2H), 3.60 (t, J = 10.6 Hz, 1H), 3.30 (s, 1H), 2.85 (s, 3H), 1.29 (d, J = 6.6 Hz, 3H) |
| 205 | 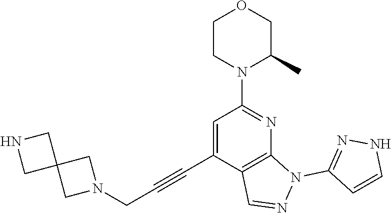 | 419.25 | ¹H NMR (399 MHz, CD3OD) δ 8.49 (s, 1H), 7.99 (s, 1H), 7.74 (s, 1H), 6.91 (d, J = 2.0 Hz, 1H), 6.84 (s, 1H), 4.67-4.41 (m, 2H), 4.19 (s, 4H), 4.04 (dd, J = 26.1, 12.9 Hz, 2H), 3.78 (dd, J = 27.6, 11.1 Hz, 2H), 3.65-3.56 (m, 7H), 1.28 (d, J = 6.5 Hz, 2H) |
| 206 | 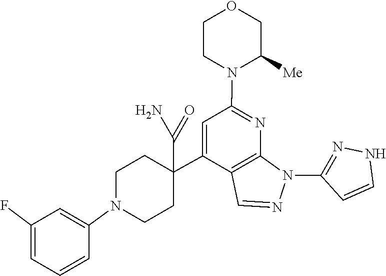 | 487.25 | ¹H NMR (400 MHz, CD3OD) δ 8.22 (s, 1H), 7.75 (s, 1H), 7.24 (s, 1H), 6.93 (s, 1H), 6.86 (d, J = 7.6 Hz, 2H), 6.80 (d, J = 12.3 Hz, 1H), 6.57 (s, 1H), 4.55 (s, 1H), 4.11 (d, J = 13.8 Hz, 1H), 4.02 (d, J = 10.4 Hz, 1H), 3.98-3.91 (m, 2H), 3.79 (dt, J = 13.3, 12.3 Hz, 2H), 3.62 (t, J = 11.0 Hz, 1H), 3.35 (d, J = 12.3 Hz, 1H), 3.26-3.19 (m, 2H), 2.41 (s, 4H), 1.31 (d, J = 6.6 Hz, 3H) |
| 207 | 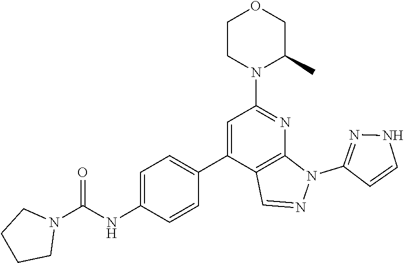 | 393.20 | ¹H NMR (400 MHz, CD3OD) δ 8.09 (s, 1H), 7.75-7.68 (m, 3H), 7.63 (d, J = 8.5 Hz, 2H), 6.97 (s, 1H), 6.80 (s, 1H), 4.59 (s, 1H), 4.14 (d, J = 12.9 Hz, 1H), 4.02 (d, J = 7.9 Hz, 1H), 3.80 (d, J = 7.0 Hz, 2H), 3.62 (s, 1H), 3.48 (s, 4H), 3.32 (s, 1H), 1.97 (s, 4H), 1.31 (d, J = 6.7 Hz, 3H) |
| 208 | 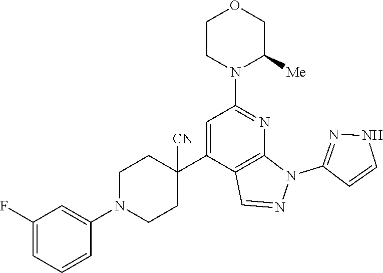 | 487.25 | ¹H NMR (400 MHz, CD3OD) δ 8.22 (s, 1H), 7.75 (s, 1H), 7.24 (s, 1H), 6.93 (s, 1H), 6.86 (d, J = 7.6 Hz, 2H), 6.80 (d, J = 12.3 Hz, 1H), 6.57 (s, 1H), 4.55 (s, 1H), 4.11 (d, J = 13.8 Hz, 1H), 4.02 (d, J = 10.4 Hz, 1H), 3.98-3.91 (m, 2H), 3.79 (dt, J = 13.3, 12.3 Hz, 2H), 3.62 (t, J = 11.0 Hz, 1H), 3.35 (d, J = 12.3 Hz, 1H), 3.26-3.19 (m, 2H), 2.41 (s, 4H), 1.31 (d, J = 6.6 Hz, 3H) |
| 209 | 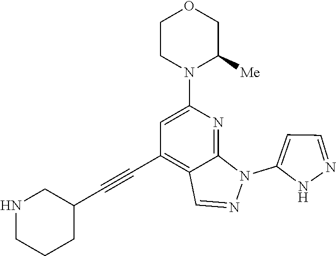 | 392.20 | ¹H NMR (400 MHz, CD3OD) δ 7.95 (s, 1H), 7.72 (s, 1H), 6.90 (s, 1H), 6.78 (s, 1H), 4.45 (d, J = 5.0 Hz, 1H), 4.02 (dd, J = 25.4, 11.2 Hz, 2H), 3.76 (q, J = 11.6 Hz, 2H), 3.59 (t, J = 11.6 Hz, 1H), 3.22 (dd, J = 23.6, 11.4 Hz, 2H), 2.91 (d, J = 12.9 Hz, 1H), 2.85-2.74 (m, 2H), 2.69 (t, J = 10.8 Hz, 1H), 2.11 (d, J = 13.3 Hz, 1H), 1.83- 1.66 (m, 2H), 1.55 (d, J = 10.3 Hz, 1H), 1.27 (d, J = 6.6 Hz, 3H) |
| 210 | 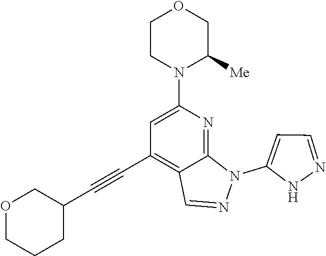 | 393.20 | ¹H NMR (400 MHz, DMSO-d6) δ 7.97 (s, 1H), 7.82 (s, 1H), 6.80 (s, 1H), 6.74 (s, 1H), 4.42 (s, 1H), 4.00 (s, 1H), 3.91 (s, 2H), 3.70 (d, J = 11.2 Hz, 2H), 3.47 (d, J = 9.7 Hz, 2H), 3.12 (s, 3H), 2.88 (s, 1H), 2.05 (s, 2H), 1.70 (s, 2H), 1.20 (s, 3H) |
| 211 | 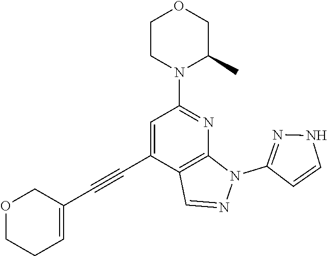 | 391.25 | ¹H NMR (400 MHz, CD3OD) δ 7.98 (s, 1H), 7.74 (s, 1H), 6.90 (s, 1H), 6.82 (s, 1H), 6.50 (s, 1H), 4.59 (s, 1H), 4.48 (s, 1H), 4.23 (d, J = 2.0 Hz, 2H), 4.11-4.05 (m, 2H), 4.00 (d, J = 9.4 Hz, 1H), 3.82-3.78 (m, 3H), 3.74 (d, J = 10.8 Hz, 1H), 3.60 (t, J = 10.0 Hz, 1H), 2.31 (s, 2H), 1.28 (d, J = 6.5 Hz, 7H) |
| 212 | 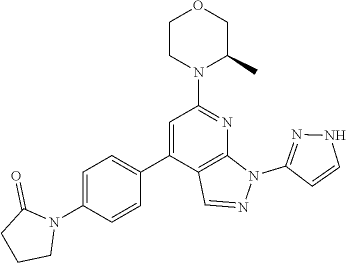 | 444.38 | ¹H NMR (400 MHz, DMSO-d6) δ 12.85 (s, 1H), 8.19 (s, 1H), 7.92-7.86 (q, J = 9.0 Hz, 5H), 6.93 (s, 1H), 6.83 (d, J = 2.2 Hz, 1H), 4.59-4.57 (d, J = 6.6 Hz, 1H), 4.17-4.14 (d, J = 12.3 Hz, 1H), 4.01-3.98 (m, 1H), 3.94-3.91 (t, J = 7.0 Hz, 2H), 3.80-3.77 (d, J = 11.3 Hz, 1H), 3.70-3.66 (dd, J = 11.4, 2.7 Hz, 1H), 3.55-3.50 (dd, J = 11.7, 9.1 Hz, 1H), 3.25-3.19 (m, 1H), 2.58-2.54 (t, J = 8.0 Hz, 2H), 2.15-2.07 (m, 2H), 1.23-1.22 (d, J = 6.6 Hz, 3H) |
| 213 | 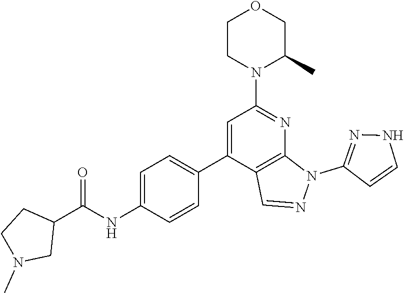 | 487.25 | ¹H NMR (400 MHz, DMSO-d6) δ 12.85 (s, 1H), 10.19 (s, 1H), 8.21 (s, 1H), 7.83 (q, J = 8.8 Hz, 5H), 6.90 (s, 1H), 6.83 (d, J = 2.2 Hz, 1H), 4.58 (d, J = 4.7 Hz, 1H), 4.15 (d, J = 12.3 Hz, 1H), 3.99 (dd, J = 11.1, 2.8 Hz, 1H), 3.78 (d, J = 11.3 Hz, 1H), 3.68 (dd, J = 11.4, 2.6 Hz, 1H), 3.52 (td, J = 11.8, 2.7 Hz, 1H), 3.26-3.10 (m, 3H), 2.94 (t, J = 8.7 Hz, 1H), 2.68 (dt, J = 16.2, 7.3 Hz, 2H), 2.34 (s, 3H), 2.05 (dd, J = 14.3, 7.1 Hz, 2H), 1.22 (d, J = 6.7 Hz, 3H) |
| 214 | 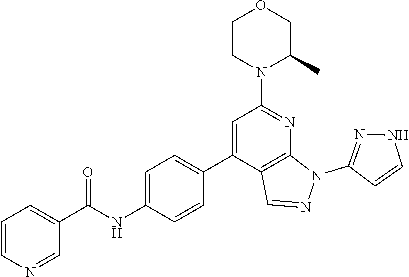 | 481.17 | ¹H NMR (400 MHz, DMSO-d6) δ 12.85 (s, 1H), 10.69 (s, 1H), 9.16 (d, J = 1.6 Hz, 1H), 8.84- 8.76 (m, 1H), 8.39-8.31 (m, 1H), 8.24 (s, 1H), 8.02 (d, J = 8.7 Hz, 2H), 7.93 (d, J = 8.7 Hz, 2H), 7.86 (s, 1H), 7.63-7.59 (dd, J = 7.8, 4.8 Hz, 1H), 6.95 (s, 1H), 6.85 (d, J = 2.2 Hz, 1H), 4.60-4.59 (d, J = 5.4 Hz, 1H), 4.19-4.15 (d, J = 13.2 Hz, 1H), 4.01-3.99 (d, J = 8.4 Hz, 1H), 3.81-3.78 (d, J = 11.3 Hz, 1H), 3.70-3.68 (d, J = 8.7 Hz, 1H), 3.56-3.51 (t, J = 10.4 Hz, 1H), 3.27-3.19 (td, J = 12.8, 3.4 Hz, 1H), 1.24 (d, J = 6.6 Hz, 3H) |
| 215 | 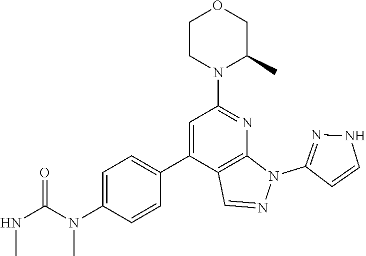 | 447.22 | ¹H NMR (400 MHz, DMSO-d6) δ 12.86 (s, 1H), 8.19 (s, 1H), 7.88-7.86 (m, J = 8.5 Hz, 3H), 7.47-7.44 (d, J = 8.5 Hz, 2H), 6.92 (s, 1H), 6.84- 6.83 (d, J = 2.1 Hz, 1H), 6.29-6.28 (d, J = 4.4 Hz, 1H), 4.57 (d, J = 6.4 Hz, 1H), 4.18-4.15 (d, J = 12.2 Hz, 1H), 4.01-3.98 (d, J = 8.1 Hz, 1H), 3.80-3.77 (d, J = 11.2 Hz, 1H), 3.69-3.67 (d, J = 8.5 Hz, 1H), 3.55-3.50 (t, J = 10.4 Hz, 1H), 3.23 (s, 3H), 3.22-3.18 (m, 1H), 2.61 (d, J = 4.3 Hz, 3H), 1.23 (s, 3H) |
| 216 | 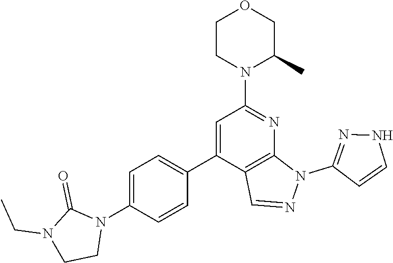 | 473.39 | ¹H NMR (400 MHz, DMSO-d6) δ 12.83 (s, 1H), 8.20 (s, 1H), 7.86 (d, J = 8.8 Hz, 3H), 7.78 (d, J = 8.9 Hz, 2H), 6.90 (s, 1H), 6.83 (d, J = 2.1 Hz, 1H), 4.58 (d, J = 5.5 Hz, 1H), 4.15 (d, J = 12.3 Hz, 1H), 4.05-3.95 (m, 1H), 3.92-3.85 (m, 2H), 3.78 (d, J = 11.2 Hz, 1H), 3.68 (dd, J = 11.3, 2.6 Hz, 1H), 3.54-3.48 (m, 3H), 3.29-3.24 (m, 2H), 3.20 (dd, J = 12.7, 3.6 Hz, 1H), 1.22 (d, J = 6.7 Hz, 3H), 1.11 (t, J = 7.2 Hz, 3H) |
| 217 | 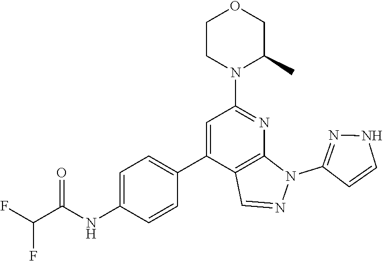 | 454.27 | ¹H NMR (400 MHz, DMSO-d6) δ 12.87 (s, 1H), 11.02 (s, 1H), 8.22 (s, 1H), 7.97-7.80 (m, 5H), 6.94 (s, 1H), 6.84 (d, J = 2.1 Hz, 1H), 6.45 (t, J = 53.7 Hz, 1H), 4.58 (d, J = 6.3 Hz, 1H), 4.16 (d, J = 13.3 Hz, 1H), 4.00 (d, J = 8.7 Hz, 1H), 3.79 (d, J = 11.3 Hz, 1H), 3.68 (d, J = 9.2 Hz, 1H), 3.53 (t, J = 10.6 Hz, 1H), 3.22 (td, J = 13.0, 3.5 Hz, 1H), 1.23 (d, J = 6.6 Hz, 3H) |
| 218 | 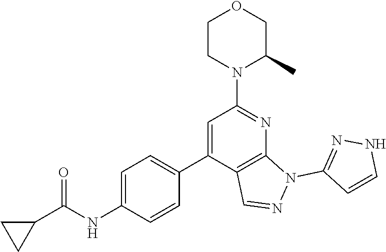 [SMILES: C[C@@H]1COCCN1c1cc(-c2ccc(NC(=O)C3CC3)cc2)c2cnn(-c3cc[nH]n3)c2n1] | 444.20 | ¹H NMR (400 MHz, DMSO-d6) δ 12.82 (s, 1H), 10.42 (s, 1H), 8.17 (s, 1H), 7.79 (q, J = 8.9 Hz, 5H), 6.87 (s, 1H), 6.80 (s, 1H), 4.53 (s, 1H), 4.11 (d, J = 12.8 Hz, 1H), 3.95 (d, J = 10.6 Hz, 1H), 3.74 (d, J = 11.5 Hz, 1H), 3.64 (d, J = 11.0 Hz, 1H), 3.49 (t, J = 10.9 Hz, 1H), 3.18 (t, J = 11.4 Hz, 1H), 1.80 (s, 1H), 1.19 (d, J = 6.4 Hz, 3H), 0.80 (d, J = 6.9 Hz, 4H) |
| 219 | 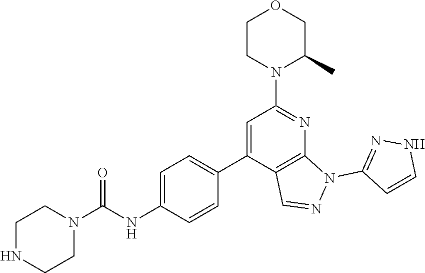 [SMILES: C[C@@H]1COCCN1c1cc(-c2ccc(NC(=O)N3CCNCC3)cc2)c2cnn(-c3cc[nH]n3)c2n1] | 488.25 | ¹H NMR (400 MHz, DMSO-d6) δ 8.70 (s, 1H), 8.22 (s, 1H), 8.17 (s, 1H), 7.81 (s, 1H), 7.75 (d, J = 8.7 Hz, 2H), 7.65 (d, J = 8.7 Hz, 2H), 6.85 (s, 1H), 6.80 (s, 1H), 4.54 (s, 1H), 4.11 (d, J = 13.7 Hz, 1H), 3.94 (s, 1H), 3.73 (s, 1H), 3.66 (s, 1H), 3.49 (s, 2H), 3.32-3.26 (m, 4H), 3.22-3.16 (m, 2H), 2.72 (s, 4H), 1.19 (d, J = 6.6 Hz, 3H) |
| 220 | 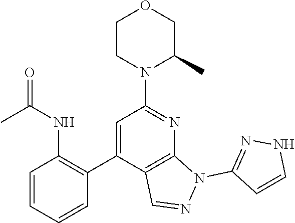 [SMILES: CC(=O)Nc1ccccc1-c1cc(N2CCOC[C@H]2C)nc2c1cnn2-c1cc[nH]n1] | 418.25 | ¹H NMR (400 MHz, DMSO-d6) δ 12.81 (s, 1H), 9.38 (s, 1H), 7.83 (s, 1H), 7.63 (d, J = 7.0 Hz, 2H), 7.48 (d, J = 7.7 Hz, 1H), 7.44 (d, J = 7.2 Hz, 1H), 7.32 (d, J = 8.2 Hz, 1H), 6.80 (s, 1H), 6.72 (s, 1H), 4.43 (s, 1H), 4.09 (d, J = 12.7 Hz, 1H), 3.96 (d, J = 11.1 Hz, 1H), 3.74 (d, J = 11.0 Hz, 1H), 3.62 (d, J = 10.7 Hz, 1H), 3.47 (t, J = 11.5 Hz, 1H), 3.18 (d, J = 11.3 Hz, 1H), 1.80 (s, 3H), 1.21 (d, J = 6.3 Hz, 3H) |
| 221 | 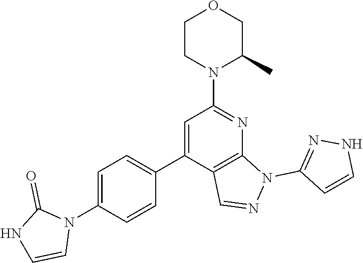 [SMILES: C[C@@H]1COCCN1c1cc(-c2ccc(-n3cc[nH]c3=O)cc2)c2cnn(-c3cc[nH]n3)c2n1] | 471.20 | ¹H NMR (400 MHz, DMSO-d6) δ 9.91 (s, 1H), 6.97 (s, 1H), 6.00 (s, 1H), 4.34 (s, 2H), 4.01 (s, 3H), 3.26 (s, 3H), 3.09 (s, 6H), 2.18-2.09 (m, 1H), 1.99-1.57 (m, 6H), 1.48 (s, 9H) |
| 222 | 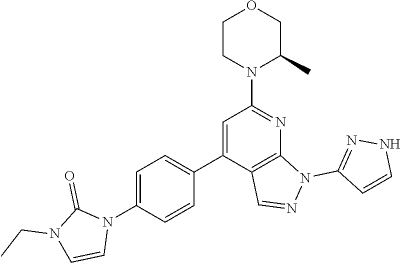 [SMILES: CCn1ccn(-c2ccc(-c3cc(N4CCOC[C@H]4C)nc4c3cnn4-c3cc[nH]n3)cc2)c1=O] | 471.30 | ¹H NMR (400 MHz, DMSO-d6) δ 12.84 (s, 1H), 8.17 (s, 1H), 7.93 (s, 3H), 7.85 (s, 1H), 7.16 (d, J = 3.2 Hz, 1H), 6.93 (s, 1H), 6.85 (d, J = 3.1 Hz, 1H), 6.81 (s, 1H), 4.56 (s, 1H), 4.14 (d, J = 16.6 Hz, 1H), 3.97 (d, J = 10.6 Hz, 1H), 3.76 (d, J = 12.1 Hz, 1H), 3.65 (s, 3H), 3.61 (s, 1H), 3.50 (t, J = 11.2 Hz, 1H), 3.19 (t, J = 10.8 Hz, 2H), 2.04- 1.90 (m, 1H), 1.21 (s, 6H) |
| 223 | 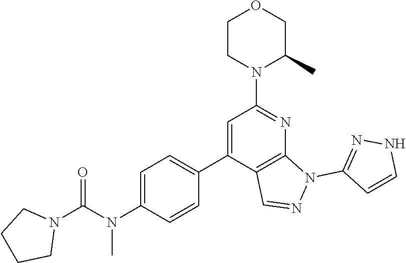 [SMILES: C[C@@H]1COCCN1c1cc(-c2ccc(N(C)C(=O)N3CCCC3)cc2)c2cnn(-c3cc[nH]n3)c2n1] | 487.35 | ¹H NMR (400 MHz, DMSO-d6) δ 12.81 (s, 1H), 8.15 (s, 1H), 7.83 (d, J = 6.5 Hz, 3H), 7.25 (d, J = 7.9 Hz, 2H), 6.88 (s, 1H), 6.80 (s, 1H), 4.54 (s, 1H), 4.12 (d, J = 13.7 Hz, 1H), 3.95 (d, J = 9.2 Hz, 1H), 3.74 (d, J = 10.9 Hz, 1H), 3.64 (d, J = 10.9 Hz, 1H), 3.49 (s, 1H), 3.19 (d, J = 12.1 Hz, 1H), 3.15 (s, 3H), 3.07 (s, 4H), 1.66 (s, 4H), 1.19 (d, J = 5.2 Hz, 3H) |
| 224 | 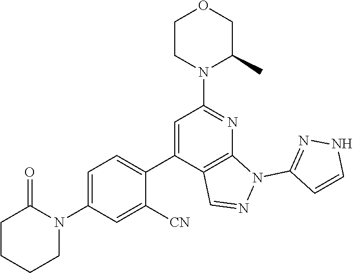 [SMILES: C[C@@H]1COCCN1c1cc(-c2ccc(N3CCCCC3=O)cc2C#N)c2cnn(-c3cc[nH]n3)c2n1] | 483.45 | ¹H NMR (400 MHz, CD3OD) δ 7.93 (d, J = 1.8 Hz, 1H), 7.90 (s, 1H), 7.80 (d, J = 8.3 Hz, 1H), 7.75 (dd, J = 8.3, 2.0 Hz, 2H), 6.97 (d, J = 2.2 Hz, 1H), 6.94 (s, 1H), 4.50 (d, J = 6.8 Hz, 1H), 4.17 (d, J = 11.7 Hz, 1H), 4.02 (dd, J = 11.4, 3.2 Hz, 1H), 3.80 (s, 1H), 3.75 (d, J = 5.7 Hz, 2H), 3.62 (td, J = 11.9, 2.8 Hz, 1H), 3.36 (dd, J = 13.0, 3.9 Hz, 1H), 2.55 (d, J = 6.1 Hz, 2H), 2.01- 1.95 (m, 4H), 1.33 (d, J = 6.7 Hz, 3H) |
| 225 | 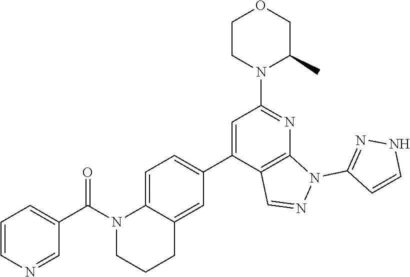 [SMILES: C[C@@H]1COCCN1c1cc(-c2ccc3c(c2)CCCN3C(=O)c2cccnc2)c2cnn(-c3cc[nH]n3)c2n1] | 521.35 | ¹H NMR (400 MHz, DMSO-d6) δ 12.82 (s, 1H), 8.60 (d, J = 3.2 Hz, 1H), 8.56 (s, 1H), 8.11 (s, 1H), 7.84 (s, 2H), 7.74 (s, 1H), 7.45 (d, J = 7.1 Hz, 2H), 7.01 (s, 1H), 6.86 (s, 1H), 6.80 (s, 1H), 4.53 (s, 1H), 4.11 (d, J = 13.3 Hz, 1H), 3.95 (d, J = 9.0 Hz, 1H), 3.81 (t, J = 6.1 Hz, 2H), 3.74 (d, J = 11.3 Hz, 1H), 3.64 (d, J = 11.6 Hz, 1H), 3.48 (t, J = 11.4 Hz, 1H), 3.18 (d, J = 10.2 Hz, 1H), 2.97 (t, J = 5.9 Hz, 3H), 2.01-1.96 (m, 2H), 1.18 (d, J = 6.1 Hz, 3H) |
| 226 | 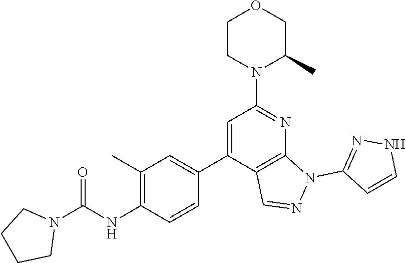 [SMILES: Cc1cc(-c2cc(N3CCOC[C@H]3C)nc3c2cnn3-c2cc[nH]n2)ccc1NC(=O)N1CCCC1] | 487.50 | ¹H NMR (400 MHz, DMSO-d6) δ 8.14 (s, 1H), 7.83 (s, 1H), 7.66-7.57 (m, 4H), 6.83 (d, J = 23.1 Hz, 2H), 4.55 (s, 1H), 4.12 (d, J = 12.7 Hz, 1H), 3.96 (d, J = 8.9 Hz, 1H), 3.75 (d, J = 11.0 Hz, 1H), 3.65 (d, J = 8.9 Hz, 1H), 3.48 (d, J = 9.9 Hz, 1H), 3.38 (s, 4H), 3.20 (d, J = 9.9 Hz, 1H), 2.30 (s, 3H), 1.85 (s, 4H), 1.19 (d, J = 6.5 Hz, 3H) |
| 227 | 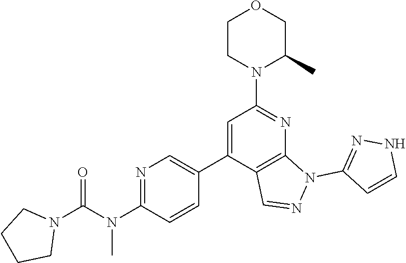 [SMILES: C[C@@H]1COCCN1c1cc(-c2ccc(N(C)C(=O)N3CCCC3)nc2)c2cnn(-c3cc[nH]n3)c2n1] | 488.25 | |
| 228 | 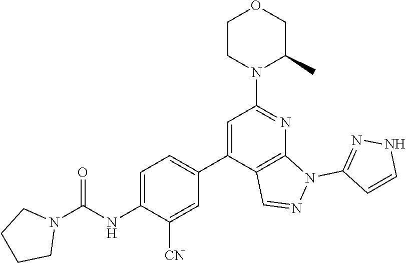 [SMILES: C[C@@H]1COCCN1c1cc(-c2ccc(NC(=O)N3CCCC3)c(C#N)c2)c2cnn(-c3cc[nH]n3)c2n1] | 498.23 | |
| 229 | 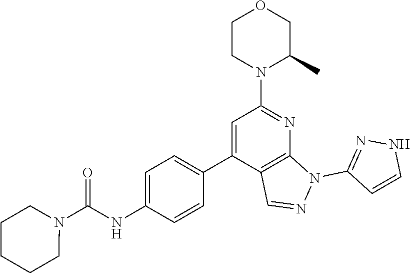 [SMILES: C[C@@H]1COCCN1c1cc(-c2ccc(NC(=O)N3CCCCC3)cc2)c2cnn(-c3cc[nH]n3)c2n1] | 487.50 | ¹H NMR (400 MHz, DMSO-d6) δ 12.80 (s, 1H), 8.67 (s, 1H), 8.16 (s, 1H), 7.83 (s, 1H), 7.74 (d, J = 8.9 Hz, 2H), 7.66 (d, J = 8.2 Hz, 2H), 6.84 (s, 1H), 6.80 (s, 1H), 4.53 (s, 1H), 4.13 (s, 1H), 3.94 (s, 1H), 3.73 (s, 1H), 3.65 (s, 1H), 3.49 (s, 1H), 3.42 (s, 4H), 3.22-3.14 (m, 1H), 1.55 (s, 2H), 1.49 (s, 4H), 1.19 (d, J = 6.1 Hz, 3H) |
| 230 | 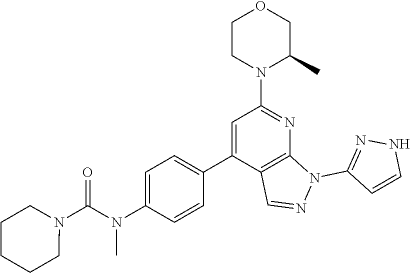 [SMILES: C[C@@H]1COCCN1c1cc(-c2ccc(N(C)C(=O)N3CCCCC3)cc2)c2cnn(-c3cc[nH]n3)c2n1] | 501.35 | ¹H NMR (400 MHz, DMSO-d6) δ 12.82 (s, 1H), 8.14 (s, 1H), 7.82 (d, J = 8.6 Hz, 3H), 7.21 (d, J = 8.6 Hz, 2H), 6.88 (s, 1H), 6.80 (s, 1H), 4.54 (s, 1H), 4.12 (d, J = 12.9 Hz, 1H), 3.95 (d, J = 8.5 Hz, 1H), 3.74 (d, J = 11.5 Hz, 1H), 3.64 (d, J = 9.0 Hz, 1H), 3.49 (t, J = 10.6 Hz, 1H), 3.21 (d, J = 1.8 Hz, 1H), 3.18-3.15 (m, 4H), 3.14 (s, 3H), 1.46 (s, 2H), 1.36 (s, 4H), 1.19 (d, J = 6.5 Hz, 3H) |
| 231 | 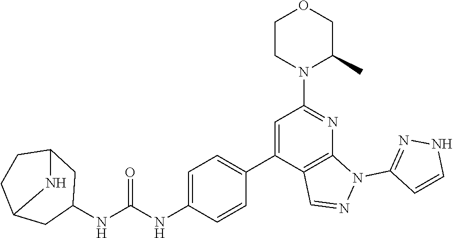 [SMILES: C[C@@H]1COCCN1c1cc(-c2ccc(NC(=O)NC3CC4CCC(C3)N4)cc2)c2cnn(-c3cc[nH]n3)c2n1] | 528.55 | ¹H NMR (400 MHz, CD3OD) δ 8.54 (s, 1H), 8.10 (s, 1H), 7.73 (dd, J = 11.4, 5.3 Hz, 3H), 7.59 (d, J = 8.6 Hz, 2H), 6.98 (d, J = 2.0 Hz, 1H), 6.81 (s, 1H), 4.62-4.57 (m, 1H), 4.15 (d, J = 12.2 Hz, 1H), 4.07-3.98 (m, 4H), 3.86-3.75 (m, 2H), 3.64 (td, J = 11.7, 2.5 Hz, 1H), 3.35 (dd, J = 12.7, 3.6 Hz, 1H), 2.39-2.26 (m, 4H), 2.20-2.06 (m, 4H), 1.32 (d, J = 6.7 Hz, 3H) |
| 232 | 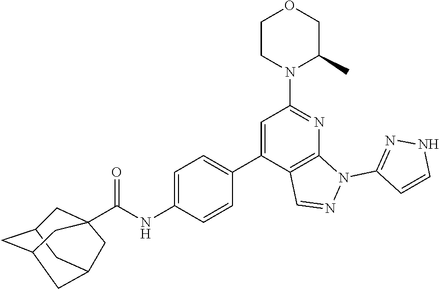 [SMILES: C[C@@H]1COCCN1c1cc(-c2ccc(NC(=O)C34CC5CC(CC(C5)C3)C4)cc2)c2cnn(-c3cc[nH]n3)c2n1] | 537.39 | ¹H NMR (500 MHz, DMSO-d6) δ 12.84 (s, 1H), 9.35 (s, 1H), 8.20 (s, 1H), 7.90 (d, J = 8.6 Hz, 2H), 7.83 (d, J = 8.6 Hz, 3H), 6.90 (s, 1H), 6.83 (s, 1H), 4.58 (d, J = 6.4 Hz, 1H), 4.15 (d, J = 13.1 Hz, 1H), 3.99 (d, J = 8.3 Hz, 1H), 3.78 (d, J = 11.3 Hz, 1H), 3.68 (d, J = 8.8 Hz, 1H), 3.52 (t, J = 10.5 Hz, 1H), 3.22 (td, J = 12.9, 3.5 Hz, 1H), 2.04 (s, 3H), 1.95 (s, 6H), 1.73 (s, 6H), 1.23 (s, 3H) |
| 233 | 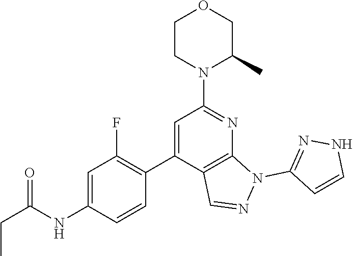 [SMILES: CCC(=O)Nc1ccc(-c2cc(N3CCOC[C@H]3C)nc3c2cnn3-c2cc[nH]n2)c(F)c1] | 450.34 | ¹H NMR (400 MHz, DMSO-d6) δ 12.80 (s, 1H), 10.31 (s, 1H), 7.94 (d, J = 2.2 Hz, 1H), 7.83 (dd, J = 13.4, 1.7 Hz, 2H), 7.68 (t, J = 8.5 Hz, 1H), 7.48 (dd, J = 8.5, 1.8 Hz, 1H), 6.85 (s, 1H), 6.82 (d, J = 2.2 Hz, 1H), 4.51 (d, J = 5.1 Hz, 1H), 4.11 (d, J = 12.4 Hz, 1H), 3.98 (dd, J = 11.2, 3.0 Hz, 1H), 3.77 (d, J = 11.3 Hz, 1H), 3.67 (dd, J = 11.4, 2.7 Hz, 1H), 3.56-3.48 (m, 1H), 3.25-3.18 (m, 1H), 2.39 (q, J = 7.5 Hz, 2H), 1.22 (d, J = 6.7 Hz, 3H), 1.11 (t, J = 7.5 Hz, 3H) |
| 234 | 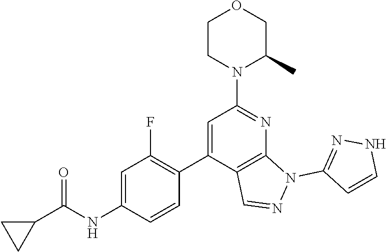 [SMILES: C[C@@H]1COCCN1c1cc(-c2ccc(NC(=O)C3CC3)cc2F)c2cnn(-c3cc[nH]n3)c2n1] | 462.3 | ¹H NMR (400 MHz, DMSO-d6) δ 12.84 (s, 1H), 10.64 (s, 1H), 7.94 (s, 1H), 7.82 (d, J = 13.7 Hz, 2H), 7.68 (t, J = 8.4 Hz, 1H), 7.48 (d, J = 8.4 Hz, 1H), 6.83 (d, J = 11.7 Hz, 2H), 4.51 (s, 1H), 4.11 (d, J = 13.5 Hz, 1H), 3.98 (d, J = 9.9 Hz, 1H), 3.76 (d, J = 11.1 Hz, 1H), 3.66 (d, J = 9.9 Hz, 1H), 3.51 (t, J = 10.9 Hz, 1H), 3.20 (t, J = 11.5 Hz, 1H), 1.89-1.75 (m, 1H), 1.20 (t, J = 11.1 Hz, 3H), 0.86 (d, J = 5.8 Hz, 4H) |
| 235 | 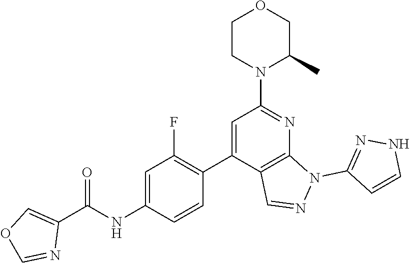 [SMILES: C[C@@H]1COCCN1c1cc(-c2ccc(NC(=O)c3cocn3)cc2F)c2cnn(-c3cc[nH]n3)c2n1] | 489.17 | ¹H NMR (400 MHz, DMSO-d6) δ 12.85 (s, 1H), 10.66 (s, 1H), 8.90 (d, J = 0.8 Hz, 1H), 8.69 (s, 1H), 8.06-7.99 (m, 1H), 7.96 (s, 1H), 7.88 (dd, J = 8.5, 1.8 Hz, 2H), 7.72 (t, J = 8.5 Hz, 1H), 6.88 (s, 1H), 6.82 (d, J = 2.1 Hz, 1H), 4.52 (s, 1H), 4.12 (d, J = 13.4 Hz, 1H), 3.98 (d, J = 8.2 Hz, 1H), 3.77 (d, J = 11.3 Hz, 1H), 3.67 (d, J = 9.1 Hz, 1H), 3.52 (t, J = 10.4 Hz, 1H), 3.21 (t, J = 11.0 Hz, 1H), 1.23 (d, J = 6.6 Hz, 3H) |
| 236 | 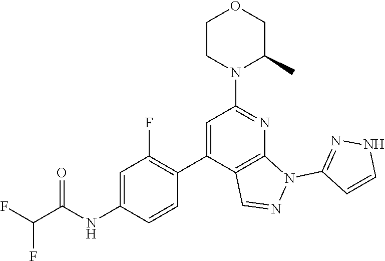 [SMILES: C[C@@H]1COCCN1c1cc(-c2ccc(NC(=O)C(F)F)cc2F)c2cnn(-c3cc[nH]n3)c2n1] | 472.16 | ¹H NMR (400 MHz, DMSO-d6) δ 12.86 (s, 1H), 11.16 (s, 1H), 8.04-7.73 (m, 4H), 7.66 (d, J = 8.4 Hz, 1H), 6.85 (d, J = 23.6 Hz, 2H), 6.46 (t, J = 53.6 Hz, 1H), 4.52 (d, J = 5.8 Hz, 1H), 4.12 (d, J = 13.3 Hz, 1H), 3.99 (dd, J = 11.0, 2.6 Hz, 1H), 3.77 (d, J = 11.3 Hz, 1H), 3.67 (dd, J = 11.3, 2.2 Hz, 1H), 3.52 (td, J = 11.9, 2.3 Hz, 1H), 3.21 (td, J = 13.0, 3.4 Hz, 1H), 1.23 (d, J = 6.6 Hz, 3H) |
| 237 | 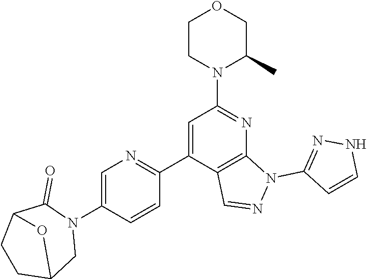 [SMILES: C[C@@H]1COCCN1c1cc(-c2ccc(N3CC4CCC(O4)C3=O)cn2)c2cnn(-c3cc[nH]n3)c2n1] | 487.21 | ¹H NMR (500 MHz, DMSO-d6) δ 12.86 (s, 1H), 8.93 (s, 1H), 8.34 (dd, J = 8.7, 2.4 Hz, 1H), 8.26- 8.19 (m, 2H), 7.87 (s, 1H), 7.02 (s, 1H), 6.83 (s, 1H), 4.88 (t, J = 5.4 Hz, 1H), 4.60 (t, J = 6.5 Hz, 2H), 4.17 (d, J = 12.3 Hz, 1H), 4.07 (dd, J = 12.4, 4.4 Hz, 1H), 3.99 (d, J = 8.2 Hz, 1H), 3.87 (d, J = 12.3 Hz, 1H), 3.78 (d, J = 11.4 Hz, 1H), 3.68 (d, J = 8.6 Hz, 1H), 3.52 (t, J = 10.4 Hz, 1H), 3.22 (t, J = 12.5 Hz, 1H), 2.12 (t, J = 8.1 Hz, 3H), 1.95 (dd, J = 20.1, 7.2 Hz, 1H), 1.23 (d, J = 6.4 Hz, 3H) |
| 238 | 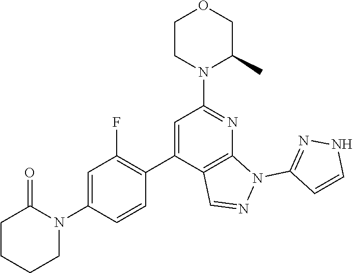 [SMILES: C[C@@H]1COCCN1c1cc(-c2ccc(N3CCCCC3=O)cc2F)c2cnn(-c3cc[nH]n3)c2n1] | 476.21 | ¹H NMR (500 MHz, DMSO-d6) δ 8.18 (s, 2H), 7.85 (s, 1H), 7.85-7.69 (m, 5H), 7.41 (d, J = 7.5 Hz, 2H), 6.93 (s, 2H), 6.83 (d, J = 1.9 Hz, 2H), 4.76 (s, 2H), 4.58 (d, J = 5.1 Hz, 2H), 4.51 (s, 2H), 4.17 (d, J = 12.9 Hz, 2H), 4.10-3.91 (m, 3H), 3.78 (d, J = 11.3 Hz, 3H), 3.68 (dd, J = 11.3, 2.6 Hz, 2H), 3.51 (dd, J = 11.7, 2.7 Hz, 2H), 3.22 (td, J = 12.8, 3.5 Hz, 4H), 2.24 (s, 8H), 1.67 (dd, J = 448.0, 25.4 Hz, 14H), 1.23 (d, J = 6.7 Hz, 7H), 1.23 (d, J = 6.7 Hz, 6H) |
| 239 | 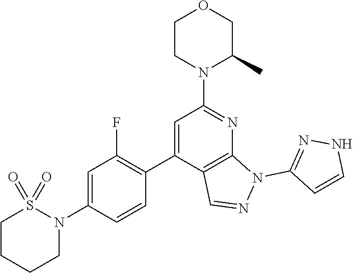 [SMILES: C[C@@H]1COCCN1c1cc(-c2ccc(N3CCCCS3(=O)=O)cc2F)c2cnn(-c3cc[nH]n3)c2n1] | 512.18 | ¹H NMR (400 MHz, DMSO-d6) δ 12.85 (s, 1H), 7.93 (d, J = 2.1 Hz, 1H), 7.86 (s, 1H), 7.75 (t, J = 8.4 Hz, 1H), 7.45-7.33 (m, 2H), 6.90 (s, 1H), 6.82 (d, J = 2.2 Hz, 1H), 4.52 (d, J = 4.8 Hz, 1H), 4.14 (d, J = 12.4 Hz, 1H), 3.98 (dd, J = 11.3, 3.0 Hz, 1H), 3.78 (dd, J = 12.7, 7.5 Hz, 3H), 3.67 (dd, J = 11.4, 2.6 Hz, 1H), 3.58-3.47 (m, 1H), 3.40 (d, J = 2.4 Hz, 1H), 3.37 (s, 1H), 3.21 (td, J = 13.0, 3.7 Hz, 1H), 2.24-2.13 (m, 2H), 1.91- 1.80 (m, 2H), 1.23 (d, J = 6.7 Hz, 3H) |
| 240 | 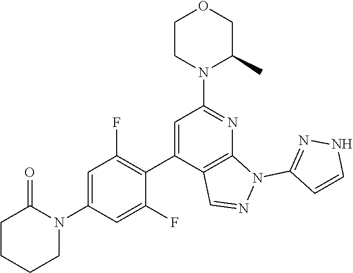 [SMILES: C[C@@H]1COCCN1c1cc(-c2c(F)cc(N3CCCCC3=O)cc2F)c2cnn(-c3cc[nH]n3)c2n1] | 494.20 | ¹H NMR (400 MHz, CD3OD) δ 7.78 (d, J = 15.1 Hz, 2H), 7.24 (d, J = 9.2 Hz, 2H), 6.98 (s, 1H), 6.87 (s, 1H), 4.52 (d, J = 6.1 Hz, 1H), 4.14 (d, J = 13.7 Hz, 1H), 4.04 (d, J = 11.0 Hz, 1H), 3.83- 3.74 (m, 4H), 3.64 (t, J = 12.1 Hz, 1H), 3.35 (s, 1H), 2.56 (t, J = 6.3 Hz, 2H), 2.04-1.95 (m, 4H), 1.32 (s, 3H) |
| 241 | 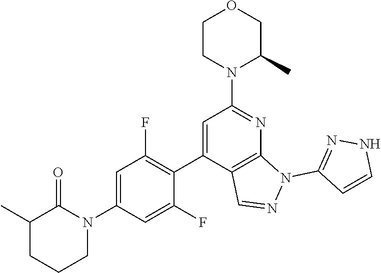 [SMILES: CC1CCCN(c2cc(F)c(-c3cc(N4CCOC[C@H]4C)nc4c3cnn4-c3cc[nH]n3)c(F)c2)C1=O] | 508.40 | ¹H NMR (400 MHz, CD3OD) δ 7.78 (d, J = 11.2 Hz, 2H), 7.21 (d, J = 9.2 Hz, 2H), 6.98 (s, 1H), 6.86 (s, 1H), 4.52 (s, 1H), 4.14 (d, J = 12.9 Hz, 1H), 4.03 (d, J = 10.7 Hz, 1H), 3.85-3.73 (m, 4H), 3.63 (d, J = 9.0 Hz, 1H), 3.34 (s, 1H), 2.61 (s, 1H), 2.11-1.99 (m, 3H), 1.70 (d, J = 9.5 Hz, 1H), 1.34-1.30 (m, 6H) |
| 242 | 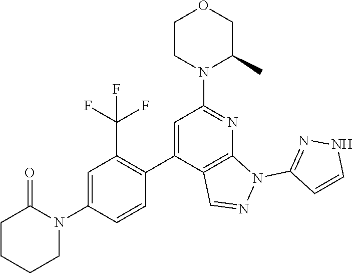 [SMILES: C[C@@H]1COCCN1c1cc(-c2ccc(N3CCCCC3=O)cc2C(F)(F)F)c2cnn(-c3cc[nH]n3)c2n1] | 525.53 | ¹H NMR (399 MHz, DMSO-d6) δ 12.85 (s, 1H), 7.85 (d, J = 8.3 Hz, 2H), 7.69 (d, J = 8.2 Hz, 1H), 7.65 (s, 1H), 7.57 (d, J = 8.1 Hz, 1H), 6.79 (s, 1H), 6.71 (s, 1H), 4.38 (s, 0H), 4.07 (d, J = 11.3 Hz, 0H), 3.93 (d, J = 8.2 Hz, 0H), 3.70 (dd, J = 10.1, 4.0 Hz, 2H), 3.61 (d, J = 8.6 Hz, 0H), 3.47 (t, J = 10.2 Hz, 0H), 3.14 (t, J = 12.2 Hz, 1H), 2.43 (t, J = 6.4 Hz, 1H), 1.87-1.83 (m, 1H), 1.19 (s, 1H), 1.16 (d, J = 6.6 Hz, 2H) |
| 243 | 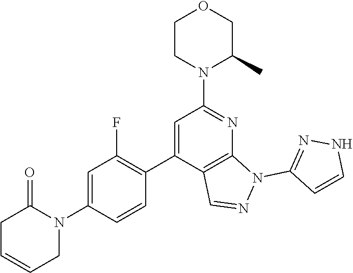 [SMILES: C[C@@H]1COCCN1c1cc(-c2ccc(N3CC=CCC3=O)cc2F)c2cnn(-c3cc[nH]n3)c2n1] | 474.20 | ¹H NMR (400 MHz, CD3OD) δ 7.91 (s, 1H), 7.74 (dd, J = 14.6, 5.7 Hz, 2H), 7.45-7.32 (m, 2H), 6.98 (s, 1H), 6.86 (s, 1H), 5.93 (s, 2H), 4.55 (d, J = 5.7 Hz, 1H), 4.39 (s, 2H), 4.15 (d, J = 14.0 Hz, 1H), 4.04 (dd, J = 12.2, 2.7 Hz, 1H), 3.87-3.76 (m, 2H), 3.64 (t, J = 10.3 Hz, 1H), 3.39-3.32 (m, 1H), 3.15 (s, 2H), 1.33 (d, J = 6.4 Hz, 3H) |
| 244 | 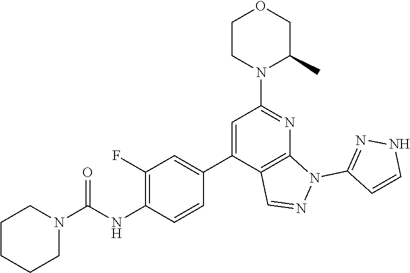 [SMILES: C[C@@H]1COCCN1c1cc(-c2ccc(NC(=O)N3CCCCC3)c(F)c2)c2cnn(-c3cc[nH]n3)c2n1] | 505.35 | ¹H NMR (400 MHz, DMSO-d6) δ 12.83 (s, 1H), 8.41 (s, 1H), 8.18 (s, 1H), 7.84 (s, 1H), 7.71 (d, J = 11.4 Hz, 1H), 7.63 (s, 2H), 6.92 (s, 1H), 6.81 (s, 1H), 4.57 (s, 1H), 4.14 (d, J = 12.8 Hz, 1H), 3.96 (d, J = 10.2 Hz, 1H), 3.75 (d, J = 11.3 Hz, 1H), 3.65 (d, J = 9.3 Hz, 1H), 3.49 (t, J = 10.4 Hz, 1H), 3.42 (s, 4H), 3.19 (s, 1H), 1.56 (s, 2H), 1.49 (s, 4H), 1.20 (d, J = 6.5 Hz, 3H) |
| 245 | 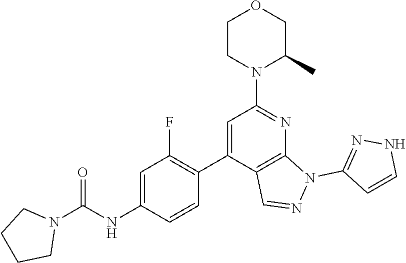 [SMILES: C[C@@H]1COCCN1c1cc(-c2ccc(NC(=O)N3CCCC3)cc2F)c2cnn(-c3cc[nH]n3)c2n1] | 491.35 | ¹H NMR (400 MHz, DMSO-d6) δ 12.83 (s, 1H), 8.56 (s, 1H), 7.91 (d, J = 2.0 Hz, 1H), 7.84 (s, 1H), 7.73 (dd, J = 14.0, 1.7 Hz, 1H), 7.57 (d, J = 8.6 Hz, 1H), 7.49 (dd, J = 8.5, 1.8 Hz, 1H), 6.80 (s, 2H), 4.47 (d, J = 5.4 Hz, 1H), 4.08 (d, J = 12.9 Hz, 1H), 3.95 (d, J = 8.6 Hz, 1H), 3.74 (d, J = 11.3 Hz, 1H), 3.64 (d, J = 9.0 Hz, 1H), 3.49 (t, J = 10.5 Hz, 1H), 3.39 (d, J = 6.3 Hz, 4H), 3.16 (d, J = 12.1 Hz, 1H), 1.85 (s, 4H), 1.19 (d, J = 6.5 Hz, 3H) |
| 246 | 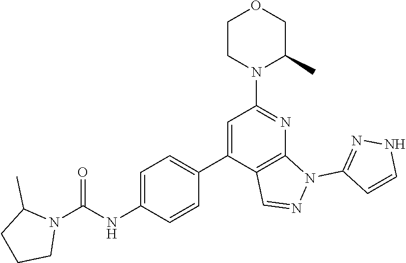 [SMILES: CC1CCCN1C(=O)Nc1ccc(-c2cc(N3CCOC[C@H]3C)nc3c2cnn3-c2cc[nH]n2)cc1] | 487.57 | ¹H NMR (400 MHz, CD3OD) δ 8.09 (s, 1H), 7.77-7.66 (m, 3H), 7.62 (d, J = 8.7 Hz, 2H), 6.97 (s, 1H), 6.78 (s, 1H), 4.56 (d, J = 7.1 Hz, 1H), 4.14 (t, J = 11.7 Hz, 2H), 4.04-3.98 (m, 1H), 3.84-3.74 (m, 2H), 3.66-3.55 (m, 2H), 3.47 (t, J = 7.9 Hz, 1H), 3.36-3.31 (m, 1H), 2.12-1.93 (m, 3H), 1.67 (s, 1H), 1.30 (d, J = 6.7 Hz, 3H), 1.24 (d, J = 6.3 Hz, 3H) |
| 247 | 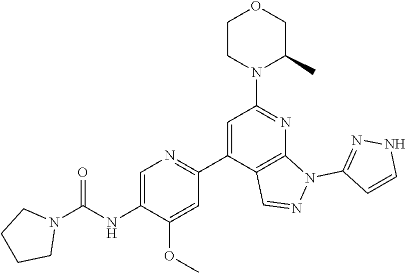 [SMILES: COc1cc(-c2cc(N3CCOC[C@H]3C)nc3c2cnn3-c2cc[nH]n2)ncc1NC(=O)N1CCCC1] | 504.24 | ¹H NMR (400 MHz, DMSO-d6) δ 12.85-12.76 (m, 1H), 9.00 (s, 1H), 8.49 (s, 1H), 7.86-7.80 (m, 1H), 7.73 (s, 1H), 7.42 (s, 1H), 7.27 (s, 1H), 6.81 (s, 1H), 4.61 (s, 1H), 4.12 (s, 1H), 4.05 (s, 3H), 4.03-3.96 (m, 1H), 3.77 (s, 1H), 3.69 (s, 1H), 3.53 (s, 1H), 3.39 (s, 2H), 3.27-3.27 (m, 1H), 3.23 (s, 2H), 1.86 (s, 4H), 1.20 (s, 3H) |
| 248 | 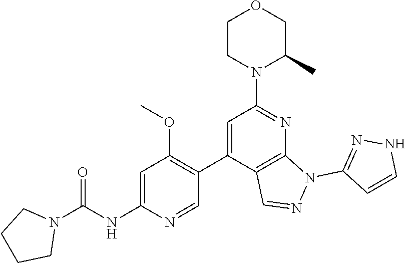 [SMILES: COc1cc(NC(=O)N2CCCC2)ncc1-c1cc(N2CCOC[C@H]2C)nc2c1cnn2-c1cc[nH]n1] | 504.24 | ¹H NMR (400 MHz, DMSO-d6) δ 8.24 (s, 1H), 7.86 (s, 1H), 7.81 (s, 1H), 7.63 (s, 1H), 6.83 (s, 1H), 6.77 (d, J = 2.3 Hz, 1H), 4.46 (d, J = 6.3 Hz, 1H), 4.06 (dd, J = 12.4, 3.3 Hz, 1H), 3.95 (dd, J = 9.3, 4.1 Hz, 1H), 3.88 (s, 3H), 3.75 (s, 1H), 3.72 (s, 1H), 3.64 (s, 1H), 3.61 (d, J = 3.3 Hz, 1H), 3.24-3.15 (m, 3H), 3.13 (s, 1H), 1.86 (s, 4H), 1.18 (d, J = 6.7 Hz, 3H) |
| 249 | 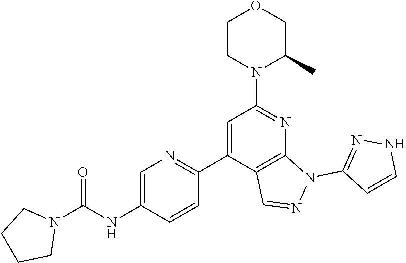 [SMILES: C[C@@H]1COCCN1c1cc(-c2ccc(NC(=O)N3CCCC3)cn2)c2cnn(-c3cc[nH]n3)c2n1] | 474.54 | ¹H NMR (400 MHz, DMSO-d6) δ 12.81 (s, 1H), 8.92 (d, J = 1.8 Hz, 1H), 8.61 (s, 1H), 8.51 (s, 1H), 8.18-8.11 (m, 2H), 7.82 (s, 1H), 7.27 (s, 1H), 6.81 (s, 1H), 4.56 (d, J = 6.7 Hz, 1H), 3.54- 3.45 (m, 1H), 3.97 (dd, J = 11.6, 2.8 Hz, 1H), 3.76 (d, J = 11.2 Hz, 1H), 3.66 (dd, J = 11.2, 2.6 Hz, 1H), 3.50 (dd, J = 13.8, 11.3 Hz, 4H), 3.25- 3.12 (m, 2H), 1.85 (s, 4H), 1.20 (s, 3H) |
| 250 | 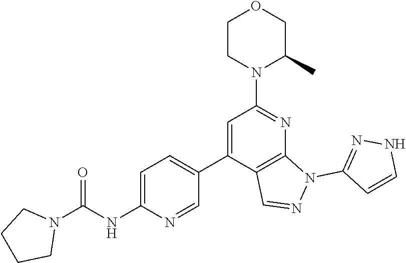 [SMILES: C[C@@H]1COCCN1c1cc(-c2ccc(NC(=O)N3CCCC3)nc2)c2cnn(-c3cc[nH]n3)c2n1] | 474.57 | ¹H NMR (400 MHz, CD3OD) δ 8.66 (d, J = 2.2 Hz, 1H), 8.17-8.08 (m, 3H), 7.75 (s, 1H), 6.97 (s, 1H), 6.86 (s, 1H), 4.61 (s, 1H), 4.16 (d, J = 12.6 Hz, 1H), 4.03 (d, J = 8.1 Hz, 1H), 3.81 (d, J = 8.1 Hz, 2H), 3.64 (s, 1H), 3.51 (t, J = 6.4 Hz, 4H), 3.34 (d, J = 3.6 Hz, 1H), 2.00 (s, 4H), 1.32 (d, J = 6.7 Hz, 3H) |
| 251 | 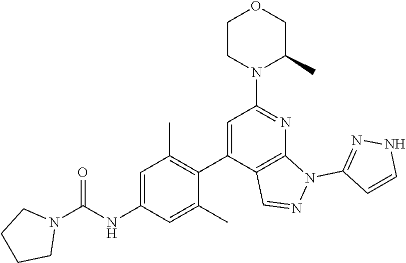 [SMILES: Cc1cc(NC(=O)N2CCCC2)cc(C)c1-c1cc(N2CCOC[C@H]2C)nc2c1cnn2-c1cc[nH]n1] | 501.26 | ¹H NMR (400 MHz, DMSO-d6) δ 12.82 (s, 1H), 8.09 (s, 1H), 7.85 (s, 1H), 7.54 (s, 1H), 7.34 (s, 2H), 6.82 (s, 1H), 6.58 (s, 1H), 4.41 (s, 1H), 4.10 (d, J = 12.5 Hz, 1H), 3.95 (d, J = 9.6 Hz, 1H), 3.71 (d, J = 10.8 Hz, 1H), 3.66 (s, 1H), 3.50 (s, 1H), 3.35 (d, J = 6.8 Hz, 5H), 3.14 (s, 1H), 1.96 (s, 7H), 1.84 (s, 4H), 1.17 (d, J = 6.4 Hz, 3H) |
| 252 | 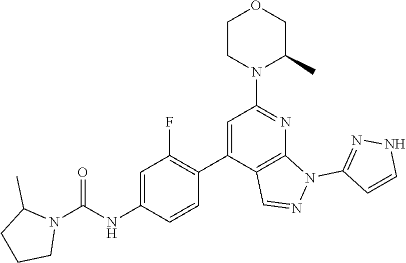 [SMILES: CC1CCCN1C(=O)Nc1ccc(-c2cc(N3CCOC[C@H]3C)nc3c2cnn3-c2cc[nH]n2)c(F)c1] | 504.24 | ¹H NMR (400 MHz, DMSO-d6) δ 12.82 (s, 1H), 8.48 (s, 1H), 7.91 (s, 1H), 7.84 (s, 1H), 7.73 (d, J = 15.9 Hz, 1H), 7.57 (d, J = 8.5 Hz, 1H), 7.49 (d, J = 7.3 Hz, 1H), 6.80 (s, 2H), 4.47 (s, 1H), 4.08 (d, J = 12.5 Hz, 2H), 3.96 (d, J = 10.9 Hz, 1H), 3.74 (d, J = 11.1 Hz, 1H), 3.64 (d, J = 11.1 Hz, 1H), 3.50 (d, J = 9.8 Hz, 2H), 1.93 (t, J = 25.5 Hz, 5H), 1.55 (s, 1H), 1.18 (s, 3H), 1.13 (d, J = 6.2 Hz, 3H) |
| 253 | 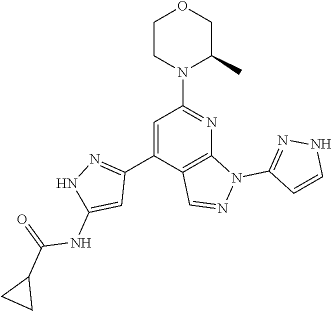 [SMILES: C[C@@H]1COCCN1c1cc(-c2cc(NC(=O)C3CC3)[nH]n2)c2cnn(-c3cc[nH]n3)c2n1] | 434.20 | ¹H NMR (400 MHz, DMSO-d6) δ 12.82 (s, 1H), 8.46 (s, 1H), 7.85 (s, 1H), 7.15 (s, 1H), 6.84- 6.75 (m, 3H), 6.20 (s, 1H), 4.60 (d, J = 5.6 Hz, 1H), 4.16 (d, J = 12.8 Hz, 1H), 4.00 (dd, J = 11.3, 3.2 Hz, 1H), 3.79 (d, J = 11.3 Hz, 1H), 3.69 (dd, J = 11.4, 2.8 Hz, 1H), 3.53 (td, J = 11.8, 2.8 Hz, 1H), 3.26-3.17 (m, 1H), 1.25- 1.20 (m, 6H), 1.13 (dt, J = 7.3, 3.5 Hz, 2H) |
| 254 | 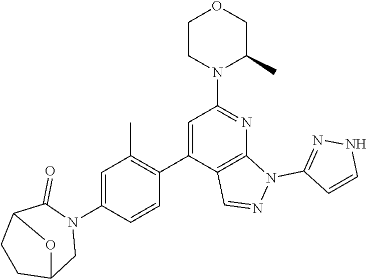 [SMILES: Cc1cc(N2CC3CCC(O3)C2=O)ccc1-c1cc(N2CCOC[C@H]2C)nc2c1cnn2-c1cc[nH]n1] | 500.38 | ¹H NMR (400 MHz, DMSO-d6) δ 12.84 (s, 1H), 7.85 (s, 1H), 7.76 (s, 1H), 7.43-7.38 (m, 2H), 7.34 (dd, J = 8.2, 1.8 Hz, 1H), 6.83 (d, J = 2.1 Hz, 1H), 6.72 (s, 1H), 4.80-4.74 (m, 1H), 4.50 (dd, J = 13.6, 5.8 Hz, 2H), 4.13 (d, J = 12.7 Hz, 1H), 4.04-3.94 (m, 2H), 3.75 (d, J = 11.3 Hz, 1H), 3.66 (dd, J = 11.3, 2.7 Hz, 1H), 3.55-3.44 (m, 2H), 3.19 (td, J = 12.9, 3.6 Hz, 1H), 2.26 (s, 3H), 2.17-2.02 (m, 4H), 1.22 (d, J = 6.7 Hz, 3H) |
| 255 | 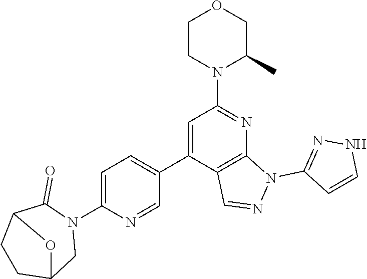 [SMILES: C[C@@H]1COCCN1c1cc(-c2ccc(N3CC4CCC(O4)C3=O)nc2)c2cnn(-c3cc[nH]n3)c2n1] | 487.22 | ¹H NMR (500 MHz, DMSO-d6) δ 12.86 (s, 1H), 8.93 (d, J = 2.4 Hz, 1H), 8.33 (dd, J = 8.7, 2.5 Hz, 1H), 8.22 (d, J = 8.6 Hz, 2H), 7.85 (s, 1H), 7.02 (s, 1H), 6.82 (d, J = 2.2 Hz, 1H), 4.94- 4.82 (m, 1H), 4.60 (d, J = 5.9 Hz, 2H), 4.17 (d, J = 12.6 Hz, 1H), 4.06 (dd, J = 12.4, 4.4 Hz, 1H), 3.99 (dd, J = 11.2, 3.2 Hz, 1H), 3.86 (d, J = 12.3 Hz, 1H), 3.78 (d, J = 11.2 Hz, 1H), 3.67 (dd, J = 11.3, 2.7 Hz, 1H), 3.52 (td, J = 11.9, 2.9 Hz, 1H), 3.22 (td, J = 12.9, 3.8 Hz, 1H), 2.19-2.06 (m, 3H), 1.94 (d, J = 11.7 Hz, 1H), 1.23 (d, J = 6.7 Hz, 3H) |
| 256 | 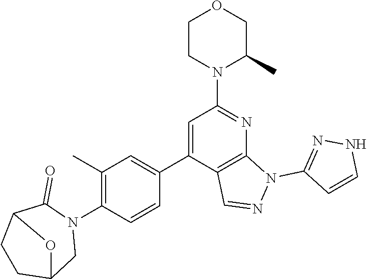 [SMILES: Cc1cc(-c2cc(N3CCOC[C@H]3C)nc3c2cnn3-c2cc[nH]n2)ccc1N1CC2CCC(O2)C1=O] | 500.23 | ¹H NMR (500 MHz, DMSO-d6) δ 8.18 (s, 2H), 7.85 (s, 1H), 7.85-7.69 (m, 5H), 7.41 (d, J = 7.5 Hz, 2H), 6.93 (s, 2H), 6.83 (d, J = 1.9 Hz, 2H), 4.76 (s, 2H), 4.58 (d, J = 5.1 Hz, 2H), 4.51 (s, 2H), 4.17 (d, J = 12.9 Hz, 2H), 4.10-3.91 (m, 3H), 3.78 (d, J = 11.3 Hz, 3H), 3.68 (dd, J = 11.3, 2.6 Hz, 2H), 3.51 (dd, J = 11.7, 2.7 Hz, 2H), 3.22 (td, J = 12.8, 3.5 Hz, 4H), 2.24 (s, 8H), 1.67 (dd, J = 448.0, 25.4 Hz, 14H), 1.23 (d, J = 6.7 Hz, 7H), 1.23 (d, J = 6.7 Hz, 6H) |
| 257 | 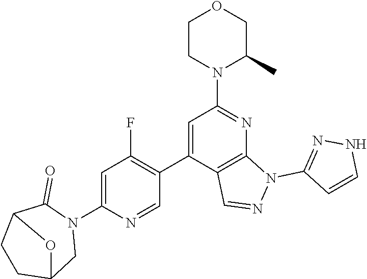 [SMILES: C[C@@H]1COCCN1c1cc(-c2cnc(N3CC4CCC(O4)C3=O)cc2F)c2cnn(-c3cc[nH]n3)c2n1] | 505.20 | ¹H NMR (400 MHz, DMSO-d6) δ 12.88 (s, 1H), 8.80 (d, J = 10.6 Hz, 1H), 8.23 (d, J = 13.0 Hz, 1H), 8.00 (s, 1H), 7.89 (s, 1H), 6.99 (s, 1H), 6.83 (s, 1H), 4.90 (t, J = 5.2 Hz, 1H), 4.65 (d, J = 5.0 Hz, 1H), 4.55 (d, J = 7.3 Hz, 1H), 4.16 (d, J = 12.4 Hz, 1H), 4.08 (dd, J = 12.5, 4.4 Hz, 1H), 4.01 (dd, J = 11.3, 2.9 Hz, 1H), 3.91 (d, J = 12.4 Hz, 1H), 3.79 (d, J = 11.4 Hz, 1H), 3.69 (dd, J = 11.5, 2.7 Hz, 1H), 3.54 (td, J = 11.7, 2.6 Hz, 1H), 3.24 (td, J = 12.6, 3.0 Hz, 1H), 2.20-2.11 (m, 3H), 2.00-1.92 (m, 1H), 1.25 (d, J = 6.6 Hz, 3H) |
| 258 | 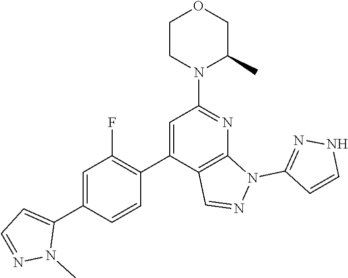 [SMILES: C[C@@H]1COCCN1c1cc(-c2ccc(-c3ccnn3C)cc2F)c2cnn(-c3cc[nH]n3)c2n1] | 458.49 | ¹H NMR (400 MHz, DMSO-d6) δ¹H 12.85 (s, 1H), 7.94 (s, 1H), 7.85 (s, 1H), 7.81 (d, J = 7.8 Hz, 1H), 7.66 (d, J = 11.3 Hz, 1H), 7.56 (d, J = 8.1 Hz, 1H), 7.51 (d, J = 1.8 Hz, 1H), 6.92 (s, 1H), 6.80 (s, 1H), 6.56 (d, J = 1.8 Hz, 1H), 4.50 (s, 1H), 4.12 (d, J = 12.1 Hz, 1H), 3.97 (s, 1H), 3.95 (s, 3H), 3.75 (d, J = 11.5 Hz, 1H), 3.65 (d, J = 8.9 Hz, 1H), 3.50 (t, J = 10.7 Hz, 1H), 3.19 (t, J = 12.9 Hz, 1H), 1.20 (s, 3H) |
| 259 | 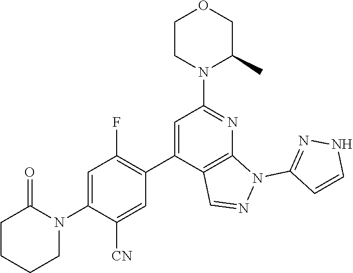 [SMILES: C[C@@H]1COCCN1c1cc(-c2cc(C#N)c(N3CCCCC3=O)cc2F)c2cnn(-c3cc[nH]n3)c2n1] | 500.21 | ¹H NMR (400 MHz, CD3OD) δ 8.17 (d, J = 7.4 Hz, 1H), 7.92 (d, J = 2.2 Hz, 1H), 7.76 (dd, J = 2.8, 1.5 Hz, 1H), 7.60 (d, J = 10.4 Hz, 1H), 6.96 (s, 1H), 6.90 (s, 1H), 4.58 (dd, J = 9.9, 5.7 Hz, 1H), 4.16 (d, J = 13.1 Hz, 1H), 4.04 (dd, J = 12.1, 2.7 Hz, 1H), 3.84-3.75 (m, 4H), 3.64 (td, J = 12.6, 3.8 Hz, 1H), 3.37 (dd, J = 13.3, 3.5 Hz, 1H), 2.61 (t, J = 6.3 Hz, 2H), 2.09-2.01 (m, 4H), 1.33 (d, J = 6.9 Hz, 3H) |
| 260 | 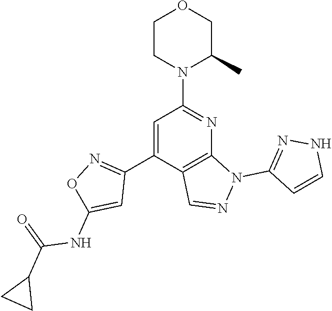 [SMILES: C[C@@H]1COCCN1c1cc(-c2cc(NC(=O)C3CC3)on2)c2cnn(-c3cc[nH]n3)c2n1] | 435.18 | ¹H NMR (400 MHz, CD3OD) δ 8.34 (s, 1H), 7.76 (s, 1H), 7.12 (s, 1H), 6.95 (s, 1H), 6.89 (s, 1H), 4.61 (s, 1H), 4.18 (d, J = 14.3 Hz, 1H), 4.04 (dd, J = 10.9, 4.0 Hz, 1H), 3.87-3.77 (m, 2H), 3.69-3.60 (m, 1H), 3.38 (dd, J = 13.2, 3.5 Hz, 1H), 1.88-1.80 (m, 1H), 1.33 (d, J = 6.8 Hz, 3H), 1.04 (dd, J = 7.3, 3.0 Hz, 2H), 0.97 (dd, J = 7.8, 3.0 Hz, 2H) |
| 261 | 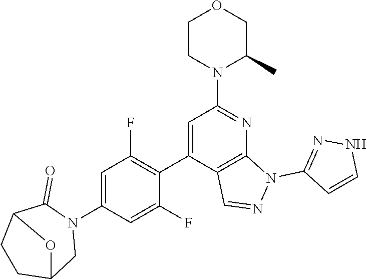 [SMILES: C[C@@H]1COCCN1c1cc(-c2c(F)cc(N3CC4CCC(O4)C3=O)cc2F)c2cnn(-c3cc[nH]n3)c2n1] | 522.52 | ¹H NMR (399 MHz, CD3OD) δ 7.76 (d, J = 7.8 Hz, 2H), 7.34 (d, J = 9.4 Hz, 2H), 6.96 (s, 1H), 6.84 (s, 1H), 4.78 (s, 1H), 4.56 (d, J = 6.1 Hz, 1H), 4.49 (s, 1H), 4.10 (t, J = 12.9 Hz, 1H), 4.01 (d, J = 9.1 Hz, 1H), 3.80 (t, J = 9.9 Hz, 1H), 3.62 (t, J = 11.1 Hz, 1H), 3.49 (d, J = 11.2 Hz, 1H), 3.35 (s, 1H), 2.23 (s, 2H), 2.06 (s, 1H), 1.32 (d, J = 6.6 Hz, 2H) |
| 262 | 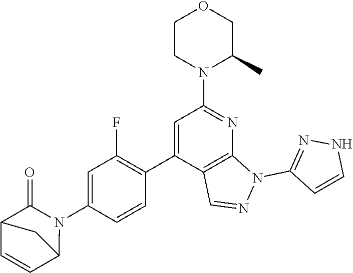 [SMILES: C[C@@H]1COCCN1c1cc(-c2ccc(N3C(=O)C4C=CC3C4)cc2F)c2cnn(-c3cc[nH]n3)c2n1] | 486.20 | ¹H NMR (400 MHz, DMSO-d6) δ 12.85 (s, 1H), 7.91 (d, J = 1.9 Hz, 1H), 7.84 (s, 1H), 7.70 (t, J = 8.6 Hz, 1H), 7.62 (dd, J = 13.1, 1.4 Hz, 1H), 7.53 (dd, J = 8.5, 2.1 Hz, 1H), 7.21 (dd, J = 5.2, 1.8 Hz, 1H), 6.86-6.78 (m, 3H), 5.18 (s, 1H), 4.50 (d, J = 6.0 Hz, 1H), 4.11 (d, J = 13.0 Hz, 1H), 3.98 (dd, J = 11.3, 3.3 Hz, 1H), 3.77 (d, J = 11.3 Hz, 1H), 3.67 (dd, J = 11.3, 2.8 Hz, 1H), 3.56-3.48 (m, 2H), 3.21 (td, J = 12.9, 3.7 Hz, 1H), 2.42 (d, J = 8.3 Hz, 1H), 2.24 (d, J = 8.3 Hz, 1H), 1.22 (d, J = 6.7 Hz, 3H) |
| 263 | 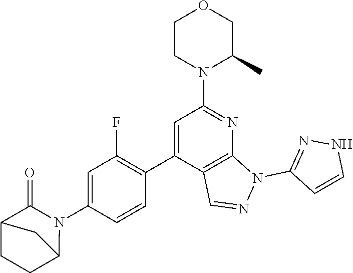 [SMILES: C[C@@H]1COCCN1c1cc(-c2ccc(N3C(=O)C4CCC3C4)cc2F)c2cnn(-c3cc[nH]n3)c2n1] | 488.21 | ¹H NMR (400 MHz, DMSO-d6) δ 12.84 (s, 1H), 7.89 (d, J = 21.3 Hz, 2H), 7.77-7.69 (m, 2H), 7.57 (dd, J = 8.6, 1.9 Hz, 1H), 6.83 (d, J = 8.9 Hz, 2H), 4.78 (s, 1H), 4.50 (d, J = 6.1 Hz, 1H), 4.10 (d, J = 12.6 Hz, 1H), 3.98 (dd, J = 11.3, 2.7 Hz, 1H), 3.77 (d, J = 11.3 Hz, 1H), 3.67 (dd, J = 11.4, 2.5 Hz, 1H), 3.52 (td, J = 11.9, 2.6 Hz, 1H), 3.26-3.16 (m, 1H), 2.89 (s, 1H), 2.04- 1.92 (m, 3H), 1.78-1.69 (m, 1H), 1.63-1.53 (m, 2H), 1.22 (d, J = 6.6 Hz, 3H) |
| 264 | 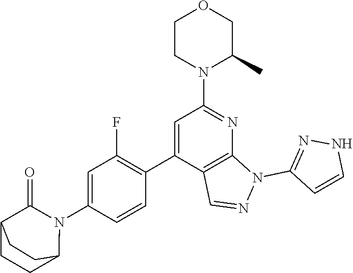 [SMILES: C[C@@H]1COCCN1c1cc(-c2ccc(N3C(=O)C4CCC3CC4)cc2F)c2cnn(-c3cc[nH]n3)c2n1] | 502.23 | ¹H NMR (400 MHz, DMSO-d6) δ 12.84 (s, 1H), 7.93 (d, J = 1.5 Hz, 1H), 7.85 (s, 1H), 7.71 (t, J = 8.5 Hz, 1H), 7.57 (dd, J = 12.7, 1.9 Hz, 1H), 7.46 (dd, J = 8.4, 1.9 Hz, 1H), 6.86 (s, 1H), 6.81 (d, J = 2.2 Hz, 1H), 4.51 (d, J = 7.2 Hz, 1H), 4.35 (s, 1H), 4.12 (d, J = 12.7 Hz, 1H), 3.98 (dd, J = 11.3, 3.1 Hz, 1H), 3.77 (d, J = 11.3 Hz, 1H), 3.67 (dd, J = 11.4, 2.7 Hz, 1H), 3.52 (td, J = 11.7, 2.6 Hz, 1H), 3.21 (td, J = 12.9, 3.6 Hz, 1H), 2.57 (s, 1H), 1.97-1.90 (m, 2H), 1.88- 1.75 (m, 6H), 1.23 (d, J = 6.6 Hz, 3H) |
| 265 | 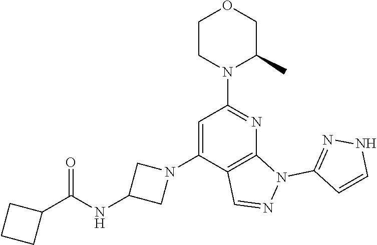 [SMILES: C[C@@H]1COCCN1c1cc(N2CC(NC(=O)C3CCC3)C2)c2cnn(-c3cc[nH]n3)c2n1] | 437.23 | ¹H NMR (400 MHz, DMSO-d6) δ 8.39 (d, J = 7.2 Hz, 1H), 7.93 (s, 1H), 7.74 (s, 1H), 6.75 (s, 1H), 5.37 (s, 1H), 4.65 (s, 1H), 4.39 (d, J = 35.2 Hz, 2H), 3.96 (s, 1H), 3.87 (d, J = 14.1 Hz, 2H), 3.70 (d, J = 10.7 Hz, 1H), 3.58 (d, J = 10.7 Hz, 2H), 3.13 (s, 1H), 3.05 (s, 1H), 3.00-2.95 (m, 1H), 2.18-1.67 (m, 7H), 1.11 (d, J = 6.6 Hz, 3H) |
| 266 | 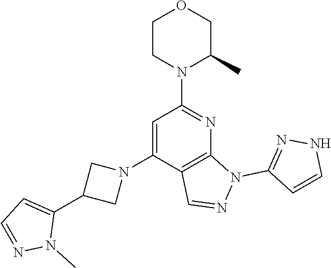 [SMILES: C[C@@H]1COCCN1c1cc(N2CC(c3ccnn3C)C2)c2cnn(-c3cc[nH]n3)c2n1] | 420.22 | ¹H NMR (400 MHz, dmso) δ 12.69 (s, 1H), 7.94 (s, 1H), 7.77 (s, 1H), 7.62 (d, J = 2.1 Hz, 1H), 6.77 (s, 1H), 6.23 (d, J = 2.1 Hz, 1H), 5.39 (s, 1H), 4.53 (s, 2H), 4.36 (s, 1H), 4.18 (s, 2H), 4.02 (t, J = 8.6 Hz, 1H), 3.91 (d, J = 8.7 Hz, 2H), 3.77 (s, 3H), 3.70 (d, J = 11.2 Hz, 1H), 3.60 (t, J = 7.4 Hz, 1H), 3.44 (t, J = 10.6 Hz, 1H), 3.07 (t, J = 12.9 Hz, 1H), 1.12 (d, J = 6.7 Hz, 3H) |
| 267 | 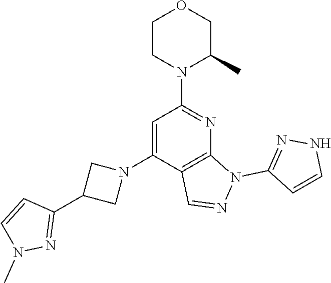 [SMILES: C[C@@H]1COCCN1c1cc(N2CC(c3ccn(C)n3)C2)c2cnn(-c3cc[nH]n3)c2n1] | 420.22 | ¹H NMR (400 MHz, DMSO-d6) δ 12.71-12.68 (m, 1H), 7.95 (s, 1H), 7.78 (s, 1H), 7.35 (s, 1H), 6.77 (s, 1H), 6.35 (s, 1H), 5.42 (s, 1H), 4.64 (s, 2H), 4.36 (s, 1H), 4.20 (s, 2H), 3.91 (s, 2H), 3.73 (s, 3H), 3.69 (s, 1H), 3.61 (s, 1H), 3.43 (t, J = 11.5 Hz, 2H), 3.08 (s, 1H), 1.12 (d, J = 6.6 Hz, 3H) |
| 268 | 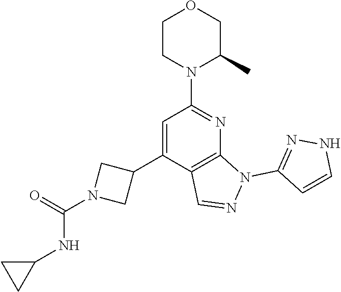 [SMILES: C[C@@H]1COCCN1c1cc(C2CN(C(=O)NC3CC3)C2)c2cnn(-c3cc[nH]n3)c2n1] | 423.22 | ¹H NMR (400 MHz, CD3OD) δ 8.04 (s, 1H), 7.74 (s, 1H), 6.93 (s, 1H), 6.69 (s, 1H), 4.54 (s, 1H), 4.42 (t, J = 7.9 Hz, 2H), 4.10 (dd, J = 5.4, 7.9 Hz, 4H), 4.04-3.99 (m, 1H), 3.85-3.73 (m, 2H), 3.61 (t, J = 10.4 Hz, 1H), 3.33 (d, J = 3.5 Hz, 1H), 2.58-2.50 (m, 1H), 1.29 (d, J = 6.8 Hz, 3H), 0.71- 0.65 (m, 2H), 0.52-0.45 (m, 2H) |
| 269 | 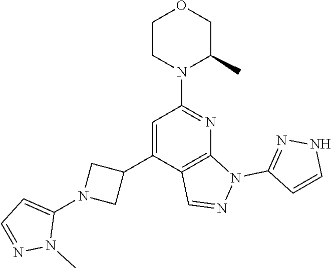 [SMILES: C[C@@H]1COCCN1c1cc(C2CN(c3ccnn3C)C2)c2cnn(-c3cc[nH]n3)c2n1] | 420.22 | ¹H NMR (400 MHz, DMSO-d6) δ 12.80 (s, 1H), 8.12 (s, 1H), 7.81 (s, 1H), 7.16 (d, J = 1.9 Hz, 1H), 6.80-6.71 (m, 2H), 5.63 (d, J = 1.9 Hz, 1H), 4.48 (s, 1H), 4.34 (dd, J = 12.8, 6.9 Hz, 2H), 4.29-4.21 (m, 1H), 4.09-3.91 (m, 4H), 3.73 (d, J = 11.0 Hz, 1H), 3.66-3.54 (m, 4H), 3.46 (t, J = 10.5 Hz, 1H), 3.15 (t, J = 11.1 Hz, 1H), 1.16 (dd, J = 6.9, 3.5 Hz, 3H) |

### Example 270

### Preparation of 3-ethynyl-1-(6-R)-3-methylmorpholinyl)-1-(1H-pyrazol-3-yl)-1H-pyrazolo[3,4-b]pyridin-4-yl)cyclopent-1-ol

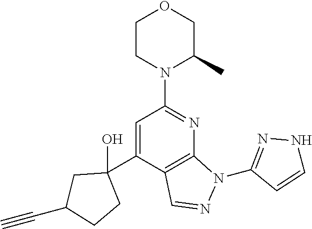 [SMILES: C#CC1CCC(O)(c2cc(N3CCOC[C@H]3C)nc3c2cnn3-c2cc[nH]n2)C1]

### Step 1: Preparation of 3-(trimethylsilylethynyl)cyclopentan-1-one

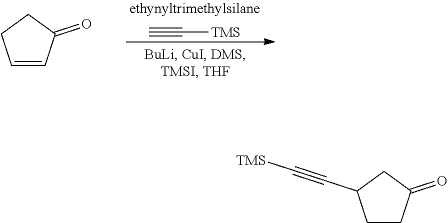

Two three-necked flasks were prepared. In flask A, ethynyltrimethylsilane (1.8 g, 18.3 mmol, 1.5 equiv.) was dissolved in tetrahydrofuran (10 mL), and the solution was cooled to −10° C. BuLi (7.4 mL, 2.5 M, 18.27 mmol, 1.5 equiv.) tetrahydrofuran solution was slowly added dropwise, and the mixture was reacted for 20 minutes for later use. In flask B, CuI (3.5 g, 18.3 mmol, 1.5 equiv.) was added to dimethyl sulfide (10 mL) solution at −10° C., to afford cuprous iodide dimethyl sulfide complex for later use. The reaction liquid in flask B was added to flask A, and the reaction mixture was cooled to −78° C. Trimethyliodosilane (3.7 g, 18.27 mmol, 1.5 equiv.) was slowly added dropwise, and the mixture was reacted for 5 minutes. Compound cyclopent-2-en-1-one (1.0 g, 12.18 mmol, 1 equiv.) in tetrahydrofuran (10 mL) was slowly added dropwise to the reaction mixture, and the resulting mixture was reacted for 30 minutes before returning to room temperature. After the reaction was completed as monitored by TLC, the reaction was quenched with saturated ammonium chloride solution, and the mixture was reacted at room temperature for 30 minutes. The reaction liquid was adjusted to PH 5 with 2 M hydrochloric acid, and the mixture was reacted for 30 minutes. The reaction liquid was extracted with ethyl acetate (50 mL), the organic phase was washed three times with saturated sodium chloride aqueous solution, dried over anhydrous sodium sulfate and concentrated in vacuo, to afford the crude product, and the crude product was purified by column chromatography (PE:EA (20:1 to 10:1) as mobile phase), to afford the target compound (1.56 g, yield: 71%).

### Step 2: Preparation of 1-(6-(R)-3-methylmorpholine-1-(2-(trimethylsilyl)ethoxy)methyl)-1H-pyrazol-3-yl)-1H-pyrazolo[3, 4-b]pyridin-4-yl)-3-(trimethylsilyl)ethynyl)cyclopentan-1-ol

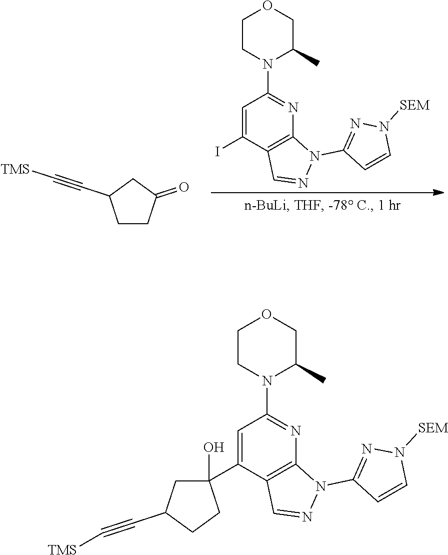

3-(Trimethylsilylethynyl)cyclopentan-1-one (400 mg, 0.74 mmol, 1.0 equiv.) and 4-iodo-1-(2-trimethylsilylethoxy)methyl-1H-pyrazol-3-yl)-1H-pyrazolo[3,4-b]pyridin-6-yl)-3-methylmorpholine (400 mg, 2.22 mmol, 3.0 equiv.) were dissolved in anhydrous tetrahydrofuran (10 mL), and the mixture was cooled to −78° C. after nitrogen replacement was performed three times. n-BuLi (0.3 mL, 2.5 M, 0.66 mmoL, 1.5 equiv.) was added dropwise, and the mixture was kept at −78° C. for 1 hour after the dropwise addition was completed. After the reaction was completed as monitored by LCMS, the reaction was quenched with saturated ammonium chloride aqueous solution, and the reaction liquid was extracted with ethyl acetate. The organic phase was washed with saturated sodium chloride aqueous solution, dried over anhydrous sodium sulfate and concentrated in vacuo. The residue was purified by column chromatography, to afford the target compound (23 mg, yield: 23%). LCMS (ESI) [M+H]⁺=594.90.

### Step 3: 3-Ethynyl-1-(6-R)-3-methylmorpholinyl)-1-(1H-pyrazol-3-yl)-1H-pyrazolo[3,4-b]pyridin-4-yl)cyclopent-1-ol

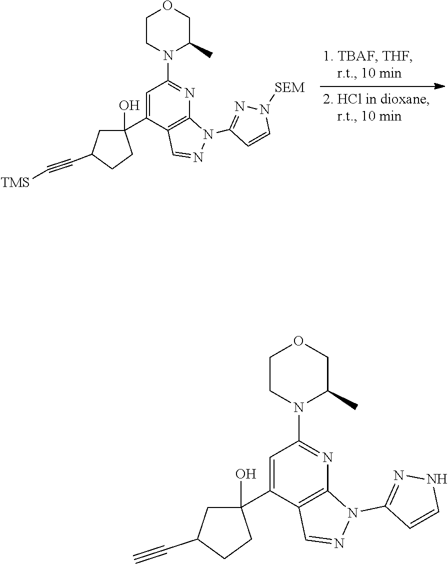

Compound 1-(6-(R)-3-methylmorpholine-1-(2-(trimethylsilyl)ethoxy)methyl)-1H-pyrazol-3-yl)-1H-pyrazolo[3,4-b]pyridin-4-yl)-3-(trimethylsilyl)ethynyl)cyclopentan-1-ol (65 mg, 0.11 mmol, 1.0 equiv.) was dissolved in dichloromethane (3 mL), TBAF (86 mg, 0.28 mmol, 2.5 equiv.) was added, and the mixture was reacted for 10 minutes. Subsequently, 4 M HCl dioxane solution was added, and the resulting mixture was reacted for 10 minutes. After the reaction was completed as monitored by TLC, sodium bicarbonate was added to neutralize the remaining hydrochloric acid, and the mixture was extracted with ethyl acetate. The organics were washed with saturated sodium chloride aqueous solution, dried over anhydrous sodium sulfate and spun to dryness, to afford the crude product, and the crude product was purified by reverse-phase preparative chromatography, to afford the target compound (11.8 mg, yield: 27%). LCMS (ESI) [M+H]⁺=392.45; ¹H NMR (400 MHz, CD3OD) δ 68.11 (s, 1H), 7.73 (s, 1H), 6.93 (s, 1H), 6.82 (s, 1H), 4.69-4.41 (m, 1H), 4.06 (dd, J=32.6, 12.3 Hz, 2H), 3.90-3.72 (m, 2H), 3.62 (s, 1H), 3.34 (d, J=3.2 Hz, 1H), 3.14 (d, J=10.0 Hz, 1H), 2.76-2.62 (m, 1H), 2.39 (d, J=2.5 Hz, 1H), 2.35-2.13 (m, 4H), 2.07 (s, 1H), 1.29 (d, J=6.7 Hz, 3H).

### Examples 271-293

The following compounds of Examples 271-293 were prepared with reference to the preparation methods of Examples 147, 200-202 and 270.

| Example | | | |
|---|---|---|---|
| No. | Structure | LCMS (ESI) [M + H]⁺ | ¹H NMR |
| 271 | 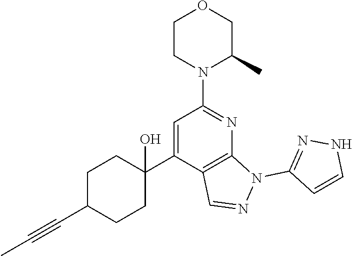 [SMILES: CC#CC1CCC(O)(c2cc(N3CCOC[C@H]3C)nc3c2cnn3-c2cc[nH]n2)CC1] | 421.23 | |
| 272 | 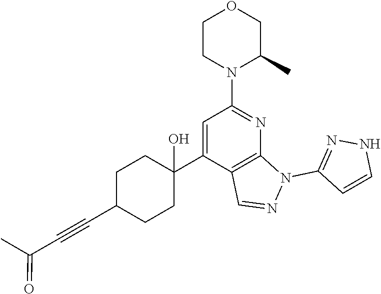 [SMILES: CC(=O)C#CC1CCC(O)(c2cc(N3CCOC[C@H]3C)nc3c2cnn3-c2cc[nH]n2)CC1] | 449.22 | |
| 273 | 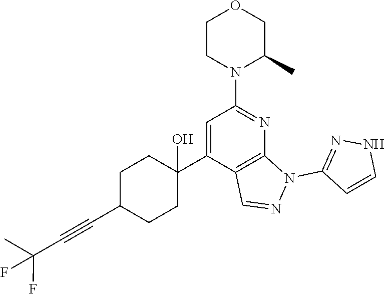 [SMILES: C[C@@H]1COCCN1c1cc(C2(O)CCC(C#CC(C)(F)F)CC2)c2cnn(-c3cc[nH]n3)c2n1] | 471.22 | |
| 274 | 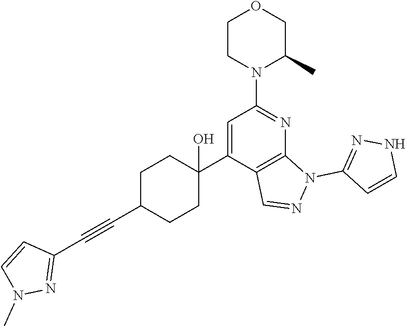 [SMILES: C[C@@H]1COCCN1c1cc(C2(O)CCC(C#Cc3ccn(C)n3)CC2)c2cnn(-c3cc[nH]n3)c2n1] | 487.25 | |
| 275 | 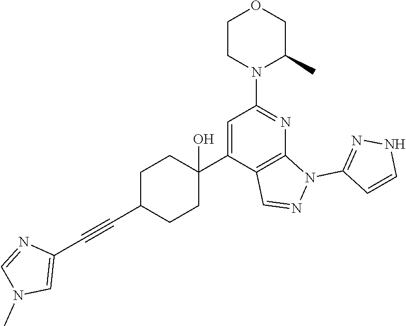 [SMILES: C[C@@H]1COCCN1c1cc(C2(O)CCC(C#Cc3cn(C)cn3)CC2)c2cnn(-c3cc[nH]n3)c2n1] | 487.25 | |
| 276 | 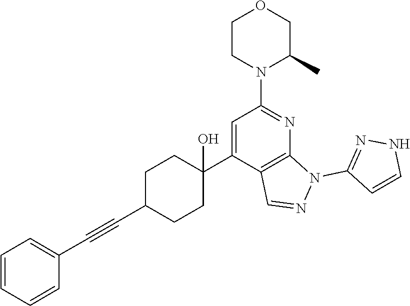 [SMILES: C[C@@H]1COCCN1c1cc(C2(O)CCC(C#Cc3ccccc3)CC2)c2cnn(-c3cc[nH]n3)c2n1] | 483.24 | |
| 277 | 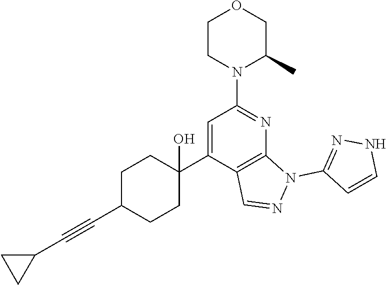 [SMILES: C[C@@H]1COCCN1c1cc(C2(O)CCC(C#CC3CC3)CC2)c2cnn(-c3cc[nH]n3)c2n1] | 447.24 | |
| 278 | 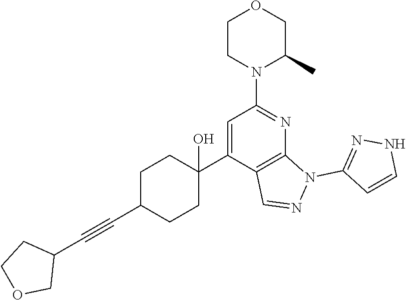 [SMILES: C[C@@H]1COCCN1c1cc(C2(O)CCC(C#CC3CCOC3)CC2)c2cnn(-c3cc[nH]n3)c2n1] | 477.25 | |
| 279 | 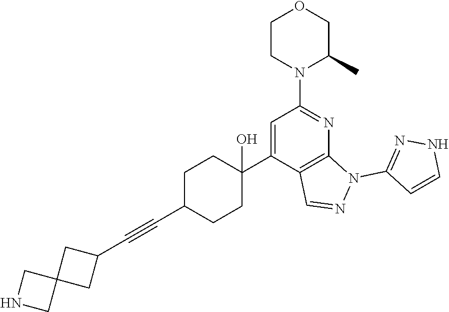 [SMILES: C[C@@H]1COCCN1c1cc(C2(O)CCC(C#CC3CC4(CNC4)C3)CC2)c2cnn(-c3cc[nH]n3)c2n1] | 502.3 | |
| 280 | 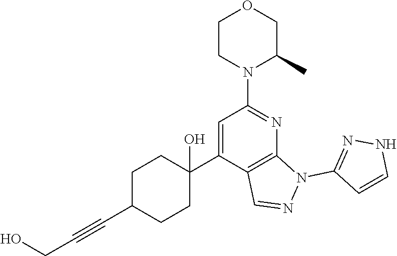 [SMILES: C[C@@H]1COCCN1c1cc(C2(O)CCC(C#CCO)CC2)c2cnn(-c3cc[nH]n3)c2n1] | 437.22 | |
| 281 | 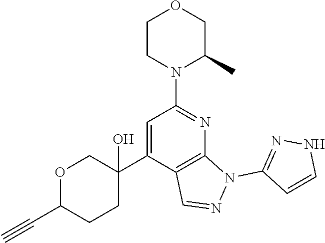 [SMILES: C#CC1CCC(O)(c2cc(N3CCOC[C@H]3C)nc3c2cnn3-c2cc[nH]n2)CO1] | 409.19 | |
| 282 | 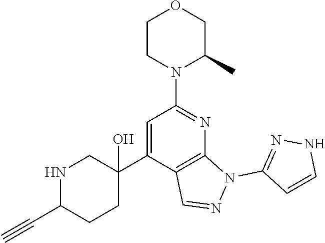 [SMILES: C#CC1CCC(O)(c2cc(N3CCOC[C@H]3C)nc3c2cnn3-c2cc[nH]n2)CN1] | 408.21 | |
| 283 | 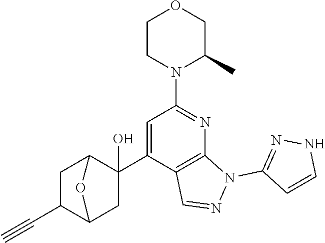 [SMILES: C#CC1CC2OC1CC2(O)c1cc(N2CCOC[C@H]2C)nc2c1cnn2-c1cc[nH]n1] | 421.19 | |
| 284 | 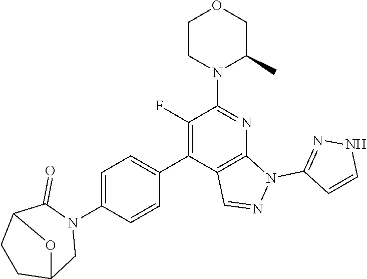 [SMILES: C[C@@H]1COCCN1c1nc2c(cnn2-c2cc[nH]n2)c(-c2ccc(N3CC4CCC(O4)C3=O)cc2)c1F] | 504.21 | |
| 285 | 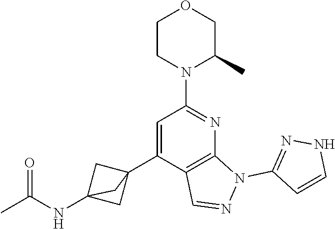 [SMILES: CC(=O)NC12CC(c3cc(N4CCOC[C@H]4C)nc4c3cnn4-c3cc[nH]n3)(C1)C2] | 408.21 | |
| 286 | 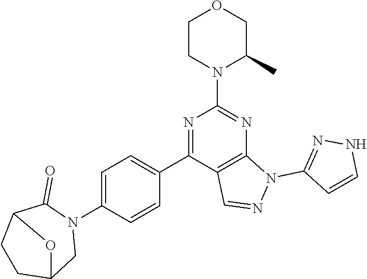 [SMILES: C[C@@H]1COCCN1c1nc(-c2ccc(N3CC4CCC(O4)C3=O)cc2)c2cnn(-c3cc[nH]n3)c2n1] | 487.21 | |
| 287 | 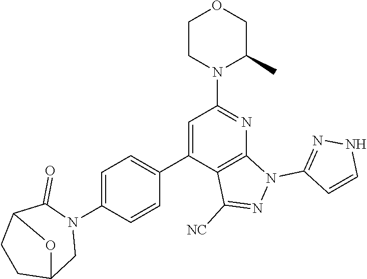 [SMILES: C[C@@H]1COCCN1c1cc(-c2ccc(N3CC4CCC(O4)C3=O)cc2)c2c(C#N)nn(-c3cc[nH]n3)c2n1] | 511.21 | |
| 288 | 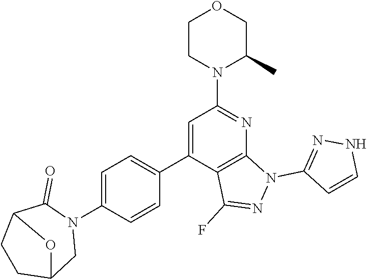 [SMILES: C[C@@H]1COCCN1c1cc(-c2ccc(N3CC4CCC(O4)C3=O)cc2)c2c(F)nn(-c3cc[nH]n3)c2n1] | 504.21 | |
| 289 | 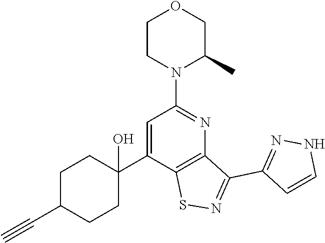 [SMILES: C#CC1CCC(O)(c2cc(N3CCOC[C@H]3C)nc3c(-c4cc[nH]n4)nsc23)CC1] | 424.17 | |
| 290 | 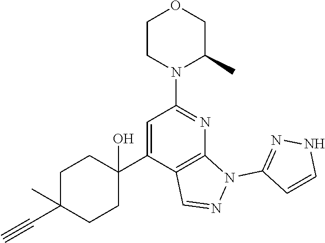 [SMILES: C#CC1(C)CCC(O)(c2cc(N3CCOC[C@H]3C)nc3c2cnn3-c2cc[nH]n2)CC1] | 421.23 | |
| 291 | 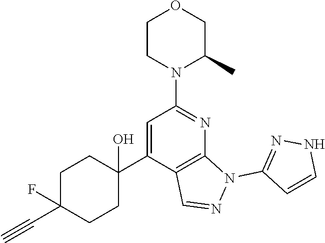 [SMILES: C#CC1(F)CCC(O)(c2cc(N3CCOC[C@H]3C)nc3c2cnn3-c2cc[nH]n2)CC1] | 425.20 | |
| 292 | 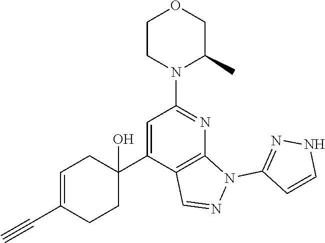 [SMILES: C#CC1=CCC(O)(c2cc(N3CCOC[C@H]3C)nc3c2cnn3-c2cc[nH]n2)CC1] | 405.20 | |
| 293 | 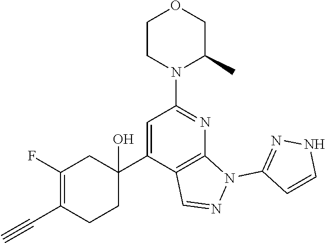 [SMILES: C#CC1=C(F)CC(O)(c2cc(N3CCOC[C@H]3C)nc3c2cnn3-c2cc[nH]n2)CC1] | 423.19 | |

### Comparative Example 1: Compound RP103

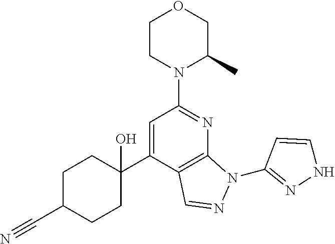 [SMILES: C[C@@H]1COCCN1c1cc(C2(O)CCC(C#N)CC2)c2cnn(-c3cc[nH]n3)c2n1]

The reference compound RP103 is prepared with reference to the preparation method of compound 103 on page 119 of the specification of patent CN 113454080 A.

Comparative example 2: compound RP3500

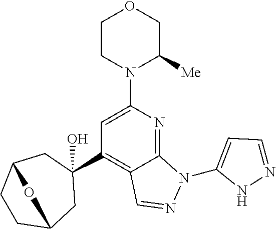 [SMILES: C[C@@H]1COCCN1c1cc([C@]2(O)C[C@H]3CC[C@@H](C2)O3)c2cnn(-c3ccn[nH]3)c2n1]

The reference compound RP3500 is prepared with reference to the preparation method of compound 121 on page 122 of the specification of patent CN 113454080 A.

### Biological Evaluation

### Test Example 1. Inhibitory Effect of Compounds of the Present Invention on ATR Enzyme

The following method is used to determine the inhibitory effect of the compounds of the present invention on the ATR enzyme. The experimental method is briefly described as follows:

### I. Experimental Materials and Instruments

1. ATR enzyme (Eurofins Pharma Discovery Services, 14-953M)

2. GST-tagged P53 protein (Eurofins Pharma Discovery Services, 14-952M)

3. 384-well plate (Geriner bio-one, 784075)

4. U-bottom 96-well plate (Geriner bio-one, 651201)

5. Anti-phospho-P53 protein antibody labeled with europium cryptate (cisbio, 61P08KAZ)

6. Anti-GST antibody linked to d2 (cisbio, 61GSTDLB)

7. ATP solution (Sigma, R⁰⁴⁴¹)

8. DTT (Sigma, D0632-259)

9. HEPES (Sigma, 15630080)

10. Microplate reader (Envision 2104 Multilabel Reader)

### II. Experimental Steps

15 nM ATR enzyme, 80 nM P53 protein, 300 nM ATP (the final concentrations were 40 nM and 150 nM, respectively), and small molecule compounds of various concentrations (the final concentrations (nM) of the ten points were 2985.0, 895.5, 298.5, 110.56, 33.17, 11.06, 4.09, 1.23, 0.41 and 0.15, respectively, and the final dimethyl sulfoxide concentration was 0.498%) were mixed and incubated at room temperature for 90 minutes. 10 μL of 2× cocktail buffer was added to the mixture of ATR, compound and substrate in the assay plate (anti-phospho-p53-Eu and anti-GST-d2 were diluted in the assay buffer). The resulting mixture was centrifuged at 1000 rpm for 30 seconds, and incubated overnight at 4° C. in the dark (a total of 20 μl in each well). The FRET signal (endpoint) was measured in the Envision instrument (HTRF 665/612 ratio was calculated at 665 nm emission and 612 nm emission). Data were processed using GraphPad software.

### III. Experimental Results

The inhibitory activity of the compounds of the present invention on the ATR enzyme can be determined by the test described above, and the measured IC50 values are shown in Table 1.

TABLE 1
IC50 values of compounds of the present
disclosure for ATR enzyme inhibition

| Example No. | IC50/nM | Example No. | IC50/nM |
|---|---|---|---|
| 1 | A | 201 | A |
| 2 | A | 202 | A |
| 3 | A | 203 | A |
| 4 | A | 204 | B |
| 5 | A | 205 | B |
| 6 | A | 206 | A |
| 7 | A | 207 | A |
| 8 | A | 208 | A |
| 9 | A | 209 | A |
| 10 | A | 210 | A |
| 11 | A | 212 | A |
| 12 | B | 213 | A |
| 13 | A | 214 | A |
| 14 | B | 215 | A |
| 15 | A | 216 | A |
| 16 | A | 217 | A |
| 17 | B | 218 | A |
| 18 | B | 219 | A |
| 30 | A | 220 | B |
| 31 | A | 221 | A |
| 32 | A | 222 | A |
| 33 | A | 223 | A |
| 58 | A | 224 | A |
| 59 | A | 225 | A |
| 60 | A | 226 | A |
| 62 | A | 227 | B |
| 65 | A | 228 | A |
| 77 | B | 229 | A |
| 78 | A | 230 | B |
| 80 | B | 231 | A |
| 84 | A | 232 | B |
| 86 | B | 233 | A |
| 145 | A | 234 | A |
| 146 | A | 235 | A |
| 147 | A | 236 | A |
| 148 | A | 237 | A |
| 149 | A | 238 | A |
| 150 | A | 239 | A |
| 151 | A | 240 | A |
| 152 | A | 241 | A |
| 153 | A | 242 | A |
| 154 | A | 243 | A |
| 155 | A | 244 | A |
| 156 | A | 245 | A |
| 157 | A | 246 | A |
| 158 | A | 247 | B |
| 159 | A | 248 | B |
| 160 | A | 249 | A |
| 161 | A | 250 | A |
| 162 | A | 251 | A |
| 163 | A | 252 | A |
| 164 | A | 253 | B |
| 165 | A | 254 | A |
| 167 | A | 255 | A |
| 168 | A | 256 | A |
| 169 | A | 257 | A |
| 171 | A | 258 | A |
| 172 | A | 259 | A |
| 183 | A | 260 | A |
| 184 | A | 261 | A |
| 187 | A | 262 | A |
| 191 | A | 263 | A |
| 192 | A | 264 | A |
| 195 | A | 265 | B |
| 197 | A | 266 | B |
| 199 | A | 267 | B |
| 200 | A | 268 | B |
| RP103 | A | 269 | A |
| RP3500 | A | 270 | A |

IC50 values for ATR enzyme inhibition: A ≤ 10 nM; 10 nM < B ≤ 50 nM; 50 nM < C ≤ 100 nM.

Conclusion: The compounds of the present disclosure have a good inhibitory activity on the ATR enzyme.

### Test Example 2. Cell Proliferation Experiment

The following method is used to evaluate the inhibitory effect of the compounds of the present invention on LoVo cell proliferation according to the IC50 values by means of detecting the intracellular ATP content. The experimental method is briefly described as follows:

### I. Experimental Materials and Instruments

1. LoVo, human colon cancer tumor cells (Co-bioer, CBP60032)

2. Fetal bovine serum (GIBCO, 10091-148)

3. F-12K medium (ATCC, 30-2004)

4. CellTite-Glo reagent (Promega, G7573)

5. 96-well cell culture plate (corning, 3599)

6. Trypsin (invitrogen, 25200-056)

7. Microplate reader (Perkin Elmer)

### II. Experimental Steps

LoVo cells were cultured in F-12K medium containing 10% FBS and passaged 2 to 3 times a week at a split ratio of 1:3 or 1:5. During passage, the cells were trypsinized and transferred to a centrifuge tube. The tube was centrifuged at 1000 rpm for 5 minutes, the supernatant medium was discarded, and fresh medium was added to resuspend the cells. 100 μL of cell suspension at a density of 1.5×104 cells/mL was added to a 96-well cell culture plate, and 100 μL of complete medium only was added to the periphery wells of the 96-well plate. The culture plate was incubated in an incubator for 24 hours (37° C., 5% CO2).

The sample to be tested was diluted to 1 mM with DMSO, diluted 3-fold serially to 8 concentrations, and prepared to 200× dilution with cell culture medium. Blank and control wells were set. 5 μL of the solution containing the compound to be tested prepared in gradient concentrations was added to 95 μL of fresh medium. 100 μL of 1× culture medium containing the compound was added to the culture plate. The culture plate was incubated in an incubator for 4 days (37° C., 5% CO2). 50 μL of CellTiter-Glo reagent was added to each well of the 96-well cell culture plate, and the plate was placed at room temperature in the dark for 5-10 min. The chemiluminescent signal values were read in PHERAstar, and data were processed using GraphPad software.

### III. Experimental Results

The inhibitory effect of the compounds of the present invention on LoVo cell proliferation can be determined by the test described above, and the measured IC50 values are shown in Table 2.

TABLE 2
IC50 values of compounds
of the present disclosure for
inhibition of LoVo cell
proliferation

| Example No. | IC50/nM |
|---|---|
| 2 | B |
| 5 | A |
| 7 | B |
| 13 | B |
| 145 | B |
| 147 | A |
| 148 | B |
| 164 | B |
| 197 | B |
| 228 | B |
| 235 | B |
| 239 | B |
| 261 | B |
| RP3500 | B |
| RP103 | B |

IC50 values for inhibition of LoVo cell proliferation: A ≤ 20 nM; 20 nM ≤ B ≤ 100 nM.

### Test Example 3. Cell Proliferation Experiment

The following method is used to evaluate the inhibitory effect of the compounds of the present disclosure on SNU-601 cell proliferation according to the IC50 values by means of detecting the intracellular ATP content. The experimental method is briefly described as follows:

### I. Experimental Materials and Instruments

1. SNU-601, human gastric cancer tumor cells (Co-bioer, CBP60507)

2. Fetal bovine serum (GIBCO, 10099-141)

3. RPMI 1640 medium (Gibco, A1049101)

4. CellTite-Glo reagent (Promega, G7573)

5. 96-well cell culture plate (corning, 3903)

6. Trypsin (Gibco, 25200056)

7. Microplate reader (TECAN, INFINITE M Nano+)

### II. Experimental Steps

SNU-601 cells were cultured in RPMI 1640 medium containing 10% FBS and passaged 2 to 3 times a week at a split ratio of 1:5 or 1:10. During passage, the cells were trypsinized and transferred to a centrifuge tube. The tube was centrifuged at 1000 rpm for 5 minutes, the supernatant medium was discarded, and fresh medium was added to resuspend the cells. 195 μL of cell suspension at a density of 5.128×10³ cells/mL was added to a 96-well cell culture plate, and 200 μL of complete medium only was added to the periphery wells of the 96-well plate. The culture plate was incubated in an incubator for 24 hours (37° C., 5% CO2).

The sample to be tested was diluted to 2 mM with DMSO, diluted 3-fold serially to 10 concentrations. Blank and control wells were set. 10 μL of the solution containing the compound to be tested prepared in gradient concentrations was added to 50 μL of fresh medium. 5 μL of the above culture medium solution containing the compound was added to the culture plate. The culture plate was incubated in an incubator for 5 days (37° C., 5% CO2). 50 μL of CellTiter-Glo reagent was added to each well of the 96-well cell culture plate after discarding 100 μL/well, and the plate was shaken at room temperature in the dark for 10 min. The chemiluminescent signal values were read in PHERAstar, and data were processed using GraphPad software.

### III. Experimental Results

The inhibitory effect of the compounds of the present invention on SNU-601 cell proliferation can be determined by the test described above, and the measured IC50 values are shown in Table 3.

TABLE 3
IC50 values of compounds of the
present disclosure for inhibition
of SNU-601 cell proliferation

| Example No. | IC50/nM |
|---|---|
| 7 | B |
| 30 | B |
| 31 | B |
| 32 | B |
| 33 | B |
| 58 | B |
| 59 | A |
| 62 | B |
| 65 | B |
| 82 | B |
| 145 | B |
| 146 | B |
| 147 | A |
| 148 | B |
| 149 | B |
| 150 | B |
| 151 | B |
| 153 | B |
| 154 | B |
| 155 | B |
| 158 | B |
| 160 | B |
| 162 | B |
| 163 | B |
| 164 | B |
| 165 | B |
| 167 | B |
| 168 | B |
| 169 | B |
| 172 | B |
| 183 | B |
| 191 | B |
| 195 | B |
| 197 | B |
| 199 | B |
| 200 | B |
| 206 | B |
| 207 | B |
| 208 | B |
| 209 | B |
| 213 | B |
| 214 | B |
| 215 | B |
| 216 | B |
| 217 | B |
| 218 | B |
| 219 | B |
| 221 | B |
| 222 | B |
| 224 | B |
| 226 | B |
| 228 | A |
| 233 | B |
| 234 | B |
| 235 | B |
| 236 | B |
| 237 | B |
| 238 | B |
| 239 | B |
| 240 | B |
| 241 | B |
| 242 | B |
| 243 | B |
| 244 | B |
| 245 | B |
| 246 | B |
| 250 | B |
| 251 | B |
| 254 | B |
| 255 | B |
| 256 | B |
| 257 | B |
| 258 | B |
| 259 | B |
| 260 | B |
| 261 | B |
| 262 | B |
| 263 | B |
| 264 | B |
| 265 | B |
| RP3500 | B |
| RP103 | B |

IC50 values for inhibition of SNU-601 cell proliferation: A ≤ 10 nM; 10 nM ≤ B ≤ 100 nM.

1-69. (canceled)

70. A compound as shown in formula (A), and a stereoisomer, an optical isomer, a pharmaceutical salt, a prodrug and a solvate thereof, 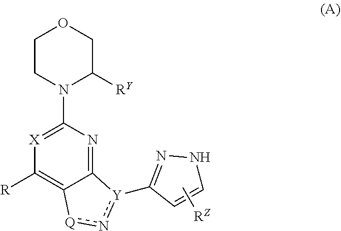
wherein,
one of the bond connecting Q and N and the bond connecting N and Y is a double bond; when the bond connecting Q and N is a double bond, the bond connecting N and Y is a single bond;
when the bond connecting N and Y is a double bond, the bond connecting Q and N is a single bond;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{Y} is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl;
R is selected from one of C₆₋₁₂ aryl, 5- to 12-membered heteroaryl, C₂₋₆ alkynyl, C₃₋₁₂ carbocyclyl and 3- to 12-membered heterocyclyl, wherein the C₆₋₁₂ aryl, 5- to 12-membered heteroaryl, C₂₋₆ alkynyl, C₃₋₁₂ carbocyclyl and 3- to 12-membered heterocyclyl are optionally substituted with one or more of the following substituents: hydroxyl, sulfhydryl, amino, carboxyl, cyano, halogen, oxo, aminoacyl, C₁₋₆ alkyl, C₂₋₆ alkenyl, C₂₋₆ alkynyl, C₃₋₁₂ carbocyclyl, C₆₋₁₂ aryl, C₁₋₆ alkoxy, C₁₋₆ alkylthio, 3- to 12-membered heterocyclyl, 5- to 12-membered heteroaryl, —C₁₋₆ alkyl-NH₂, —NHC₁₋₆ alkyl, —NH-5- to 12-membered heteroaryl, —N(C₁₋₆ alkyl)₂, —NHCOC₁₋₆ alkyl, —NHCOC₃₋₆ carbocyclyl, —NHCOC₃₋₁₂ aryl, —NHCO-3- to 12-membered heteroaryl, —NHCO-3- to 12-membered heterocyclyl, —NHCONHC₁₋₆ alkyl, —NHCONHC₃₋₁₂ carbocyclyl, —NHCONH-3- to 12-membered heterocyclyl, —CONH C₁₋₆ alkyl, —CON(C₁₋₆ alkyl)₂, —C₁₋₆ alkyl-C₃₋₁₂ carbocyclyl, —C₁₋₆ alkyl-5- to 12-membered heteroaryl, —C₁₋₆ alkyl-3- to 12-membered heterocyclyl and —C₁₋₆ alkyl-C₆₋₁₂ aryl, wherein the C₁₋₆ alkyl, C₂₋₆ alkenyl, C₂₋₆ alkynyl, C₃₋₁₂ carbocyclyl, C₆₋₁₂ aryl, C₁₋₆ alkoxy, C₁₋₆ alkylthio, 3- to 12-membered heterocyclyl, 5- to 12-membered heteroaryl, —C₁₋₆ alkyl-NH₂, —NHC₁₋₆ alkyl, —NH-5- to 12-membered heteroaryl, —N(C₁₋₆ alkyl)₂, —NHCOC₁₋₆ alkyl, —NHCOC₃₋₆ carbocyclyl, —NHCOC₃₋₁₂ aryl, —NHCO-3- to 12-membered heteroaryl, —NHCO-3- to 12-membered heterocyclyl, —NHCONHC₁₋₆ alkyl, —NHCONHC₃₋₆ carbocyclyl, —CONHC₁₋₆ alkyl, —CON(C₁₋₆ alkyl)₂, —C₁₋₆ alkyl-C₃₋₁₂ carbocyclyl, —C₁₋₆ alkyl-5- to 12-membered heteroaryl, —C₁₋₆ alkyl-3- to 12-membered heterocyclyl and —C₁₋₆ alkyl-C₆₋₁₂ aryl are optionally substituted with one or more of the following substituents: hydroxyl, sulfhydryl, amino, carboxyl, cyano, halogen, oxo, amido, aminoacyl, —SO₂NH₂, C₁₋₆ alkyl optionally substituted with halogen or hydroxyl, C₂₋₆ alkenyl optionally substituted with halogen or hydroxyl, C₂₋₆ alkynyl optionally substituted with halogen or hydroxyl, C₁₋₆ alkoxy optionally substituted with halogen or hydroxyl, —C₁₋₆ alkyl-OH optionally substituted with halogen or hydroxyl, —C₁₋₆ alkyl-O—C₁₋₆ alkyl optionally substituted with halogen or hydroxyl, C₃₋₆ cycloalkyl optionally substituted with halogen or hydroxyl, C₆₋₁₂ aryl optionally substituted with halogen or hydroxyl, —CH₂—C₆₋₁₂ aryl optionally substituted with halogen or hydroxyl, 3- to 6-membered heterocyclyl optionally substituted with halogen, hydroxyl or C₁₋₃ alkyl, 5- to 10-membered heteroaryl optionally substituted with halogen, hydroxyl or C₁₋₃ alkyl, —SONHC₁₋₆ alkyl optionally substituted with halogen or hydroxyl, —SO₂C₁₋₆ alkyl optionally substituted with halogen or hydroxyl, —COC₁₋₆ alkyl optionally substituted with halogen or hydroxyl, —COC₃₋₆ cycloalkyl optionally substituted with halogen or hydroxyl, —COC₆₋₁₂ aryl optionally substituted with halogen or hydroxyl, —NHSO₂C₁₋₆ alkyl optionally substituted with halogen or hydroxyl, —CONHC₁₋₆ alkyl optionally substituted with halogen or hydroxyl, —NHC₁₋₆ alkyl optionally substituted with halogen or hydroxyl, —N(C₁₋₆ alkyl)₂ optionally substituted with halogen or hydroxyl, and —NHC₃₋₆ cycloalkyl optionally substituted with halogen or hydroxyl;
when the bond connecting Q and N is a double bond, the bond connecting N and Y is a single bond, in which case Q and Y are each selected from CR¹ or N; wherein R¹, at each occurrence, is independently selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
when the bond connecting N and Y is a double bond, the bond connecting Q and N is a single bond, in which case Y is selected from C, and Q is selected from CR²R³ or NR⁴; wherein R², R³ and R⁴, at each occurrence, are independently selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy and 4- to 6-membered heterocyclyl;
the heteroatoms in the “heterocyclyl” and “heteroaryl” are selected from N, O or S, and the number of the heteroatoms is 1, 2, 3 or 4;
wherein the compound as shown in formula (A) is further represented by formula (A-1), (A-2), (A-3), (A-4), (A-5), (A-6) or (A-7): 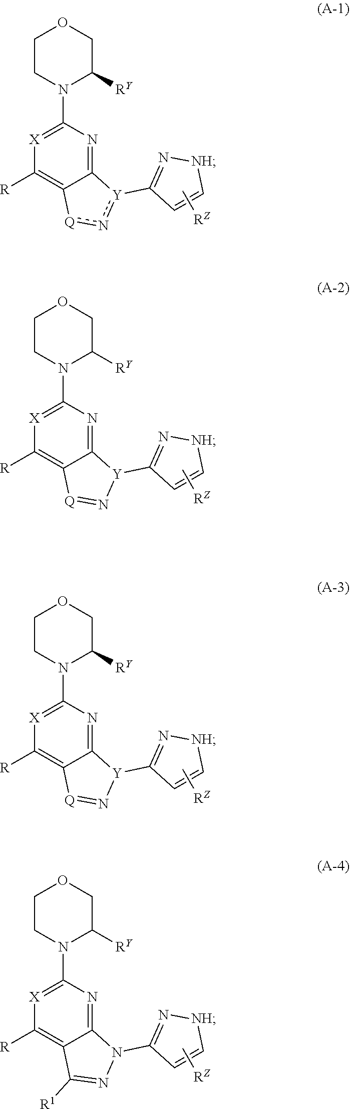 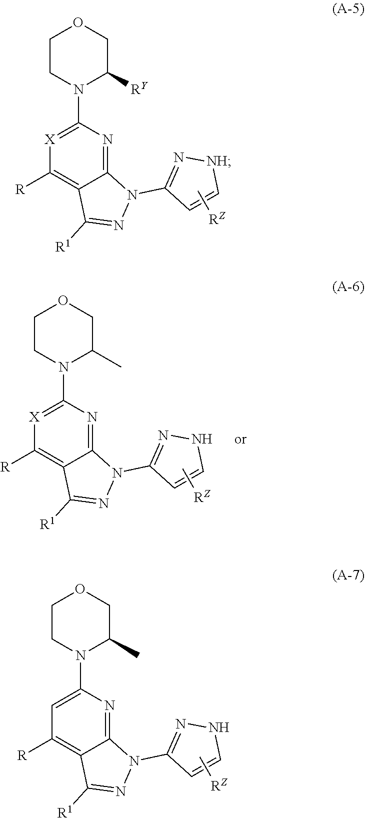
wherein the substituents in formula (A-1), (A-2), (A-3), (A-4), (A-5), (A-6) or (A-7) are as defined in formula (A).

71. The compound of claim 70, which is as shown in formula (B), and a stereoisomer, an optical isomer, a pharmaceutical salt, a prodrug and a solvate thereof, 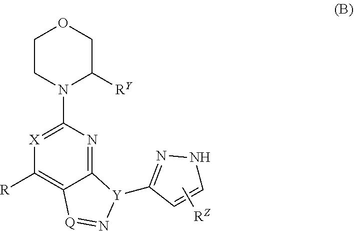
wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{Y} is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl;
R is selected from one of the following substituents: 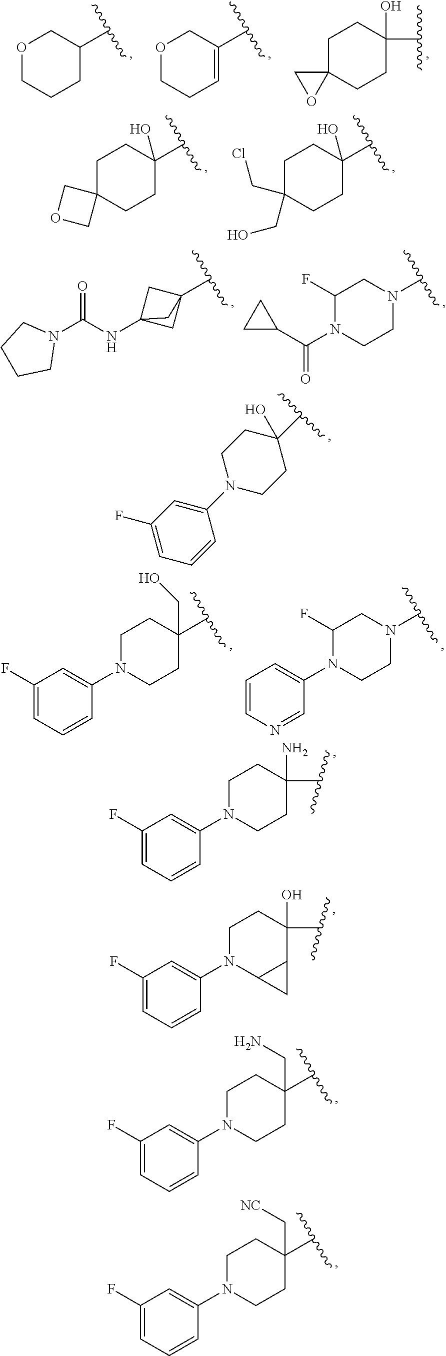 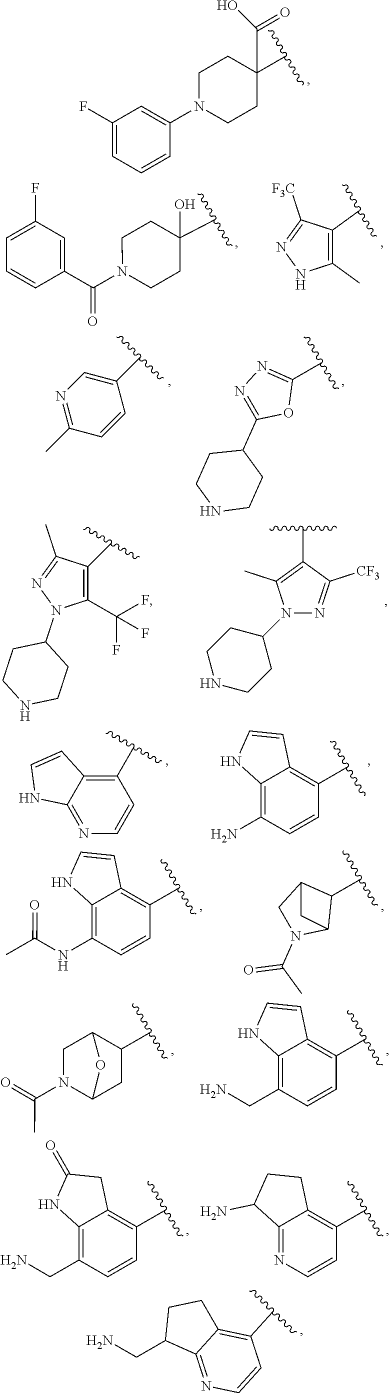 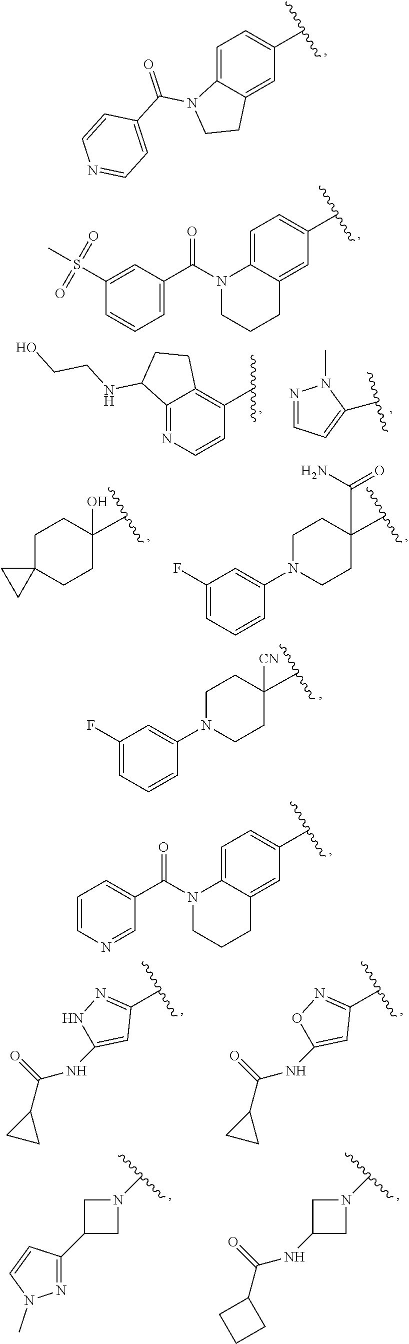 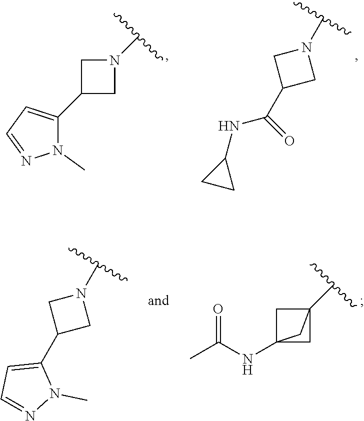
wherein the compound as shown in formula (B) is further represented by formula (B-1), (B-2), (B-3), (B-4) or (B-5): 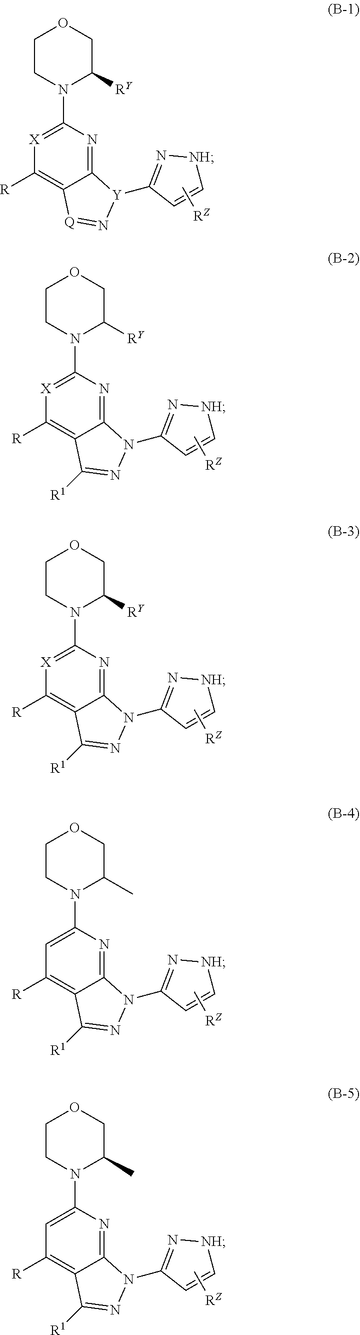
wherein the substituents in formula (B-1), (B-2), (B-3), (B-4) or (B-5) are as defined in formula (B).

72. A compound of claim 70, which is as shown in formula (C), and a stereoisomer, an optical isomer, a pharmaceutical salt, a prodrug and a solvate thereof, 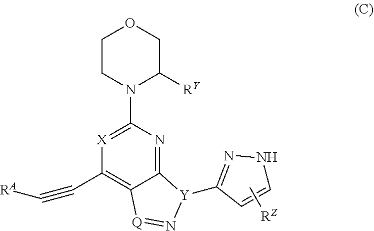
wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{Y} is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl;
R^{A} is selected from hydrogen, carboxyl, —C₁₋₆ alkyl-NH₂, —Z—C₁₋₆ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl or —CONHC₁₋₆ alkyl; wherein —Z— is selected from a bond, —C(R¹⁰)(R¹¹)—, —C(R¹²)(R¹³) C(R¹⁴)(R¹⁵)—, —N(R¹⁶)—, —O— or —S—, wherein R¹⁰, R¹¹, R¹², R¹³, R¹⁴, R¹⁵ and R¹⁶ are each independently selected from hydrogen, methyl, ethyl, hydroxyl, carboxyl, amino, amido, cyano and oxo, and when one substituent of R¹⁰ and R¹¹, R¹² and R¹³, or R¹⁴ and R¹⁵ connected to the same atom is selected from oxo, the other substituent is absent; the —C₁₋₆ alkyl-NH₂, —Z—C₁₋₆ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl and —CONHC₁₋₆ alkyl are optionally substituted with one or more of the following substituents: hydroxyl, cyano, halogen, oxo, amido, —SO₂NH₂, optionally substituted C₁₋₆ alkyl, optionally substituted C₁₋₆ alkoxy, optionally substituted —C₁₋₆ hydroxyalkyl, optionally substituted C₆₋₁₂ aryl, optionally substituted 3- to 6-membered heterocyclyl, optionally substituted 5- to 10-membered heteroaryl, optionally substituted —SONHC₁₋₆ alkyl, optionally substituted —SO₂C₁₋₆ alkyl, optionally substituted —COC₁₋₆ alkyl, optionally substituted —COC₃₋₆ cycloalkyl, optionally substituted —COC₆₋₁₂ aryl, optionally substituted —NHSO₂C₁₋₆ alkyl, and optionally substituted —CONHC₁₋₆ alkyl; the expression “optionally substituted” refers to the case of being unsubstituted or substituted with one or more of the following substituents: C₁₋₆ alkyl, hydroxyl, halogen and oxo;
the heteroatoms in the “heterocyclyl” and “heteroaryl” are selected from N, O or S, and the number of the heteroatoms is 1, 2, 3 or 4;
wherein the compound as shown in formula (C) is further represented by formula (C-1), (C-2), (C-3), (C-4) or (C-5): 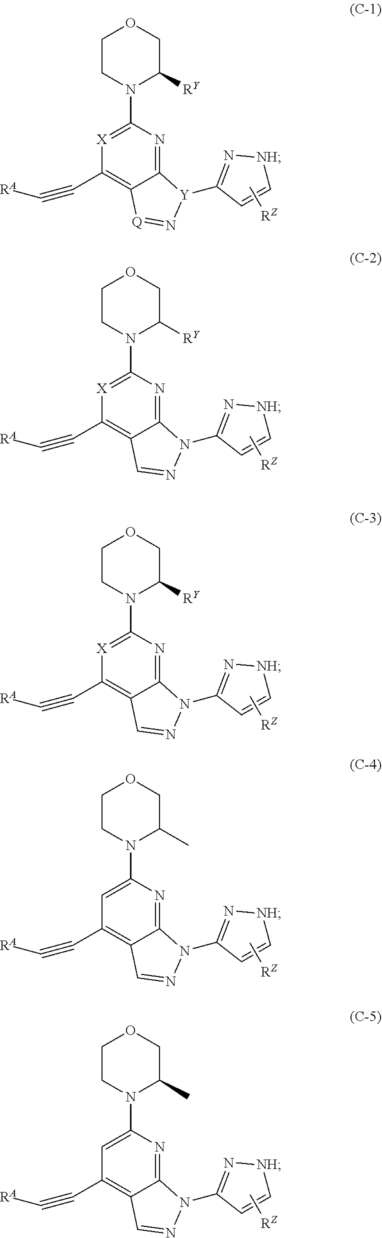
wherein the substituents in formula (C-1), (C-2), (C-3), (C-4) or (C-5) are as defined in formula (C).

73. The compound, and the stereoisomer, the optical isomer, the pharmaceutical salt, the prodrug and the solvate thereof according to claim 72, wherein Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio; or,
Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio; or,
Y is selected from N, and Q is selected from CR¹; R¹ is selected from hydrogen, halogen, cyano, amido, methyl, ethyl, methoxy, ethoxy, methylthio or ethylthio; or,
Y is selected from N, and Q is selected from CR¹; R¹ is selected from hydrogen, F, Cl, Br, cyano, amido, methyl, ethyl, methoxy or methylthio; or,
Y is selected from N, and Q is selected from CR¹; R¹ is selected from hydrogen, F, cyano, amido or methylthio; or,
Y is selected from N, and Q is selected from CR¹; R¹ is selected from hydrogen;
X is selected from CR^{X}; wherein R^{X} is selected from hydrogen, halogen, hydroxyl, sulfhydryl, amino, cyano or C₁₋₆ alkyl; or,
R^{X} is selected from hydrogen, F, Cl, Br, hydroxyl, amino, cyano or methyl; or,
R^{X} is selected from hydrogen;
R^{Y} is F, Cl, Br, methyl, ethyl, n-propyl, isopropyl or hydrogen; or,
R^{Y} is F, Cl, Br, methyl, ethyl or hydrogen; or,
R^{Y} is methyl;
the number of R^{Z} is 0, 1, 2 or 3, and R^{Z}, at each occurrence, is independently selected from F, Cl, Br, hydroxyl, cyano, amino, methyl, ethyl, monofluoromethyl, difluoromethyl or trifluoromethyl; or,
the number of R^{Z} is 0, 1 or 2, and R^{Z}, at each occurrence, is independently selected from F, Cl, Br, hydroxyl, methyl, monofluoromethyl, difluoromethyl or trifluoromethyl; or,
the number of R^{Z} is 0.

74. The compound, and the stereoisomer, the optical isomer, the pharmaceutical salt, the prodrug and the solvate thereof according to claim 72, wherein R^{A} is selected from hydrogen, carboxyl, amido, —C₁₋₄ alkyl-NH₂, —Z—C₁₋₄ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl or —CONHC₁₋₄ alkyl; wherein —Z— is selected from a bond, —C(R¹⁰)(R¹¹)—, —C(R¹²)(R¹³) C(R¹⁴)(R¹⁵)— or —N(R¹⁶)—, wherein R¹⁰, R¹¹, R¹², R¹³, R¹⁴, R¹⁵ and R¹⁶ are each independently selected from hydrogen, methyl, hydroxyl, amino, cyano and oxo, and when one substituent of R¹⁰ and R¹¹, R¹² and R¹³, or R¹⁴ and R¹⁵ connected to the same atom is selected from oxo, the other substituent is absent;
the amido, —C₁₋₄ alkyl-NH₂, —Z—C₁₋₄ alkyl, —Z—C₃₋₁₂ cycloalkyl, —Z—C₆₋₁₂ cycloalkenyl, —Z—C₆₋₁₂ aryl, —Z-3- to 12-membered heterocyclyl, —Z-5- to 12-membered heteroaryl and —CONHC₁₋₄ alkyl are optionally substituted with one or more of the following substituents: hydroxyl, cyano, halogen, oxo, amido, —SO₂NH₂, optionally substituted C₁₋₄ alkyl, optionally substituted C₁₋₄ alkoxy, optionally substituted —C₁₋₄ alkyl-OH, optionally substituted C₆₋₁₂ aryl, optionally substituted 3- to 6-membered heterocyclyl, optionally substituted 5- to 10-membered heteroaryl, optionally substituted —SONHC₁₋₄ alkyl, optionally substituted —SO₂C₁₋₄ alkyl, optionally substituted —COC₁₋₄ alkyl, optionally substituted —COC₃₋₆ cycloalkyl, optionally substituted —COC₆₋₁₂ aryl, optionally substituted —NHSO₂C₁₋₄ alkyl, and optionally substituted —CONHC₁₋₄ alkyl; the expression “optionally substituted” refers to the case of being unsubstituted or substituted with one or more of the following substituents: methyl, ethyl, n-propyl, isopropyl, hydroxyl, halogen and oxo; or,
R^{A} is selected from hydrogen, carboxyl, amido, —C₁₋₄ alkyl-NH₂, —Z—C₁₋₄ alkyl, —Z—C₃₋₆ monocyclic cycloalkyl, —Z-6- to 8-membered spirocycloalkyl, —Z—C₆ cycloalkenyl, —Z-phenyl, —Z-4- to 7-membered monocyclic heterocyclyl, —Z-6- to 8-membered bridged heterocyclyl, —Z-6- to 9-membered fused heterocyclyl, —Z-7- to 11-membered spiro heterocyclyl, —Z-5- to 6-membered monocyclic heteroaryl or —Z-7- to 9-membered fused heteroaryl, wherein —Z— is selected from a bond, —CH₂—, —CH₂CH₂—, —NH—, —CH(OH)—, —CH(CN)—, —CH(CH₃)—, —CO—, —COCH₂—, —CH₂CO—, —COCO—, —CH(OH)CH₂—, —CH₂CH(OH)—, —CH(CN)CH₂—, —CH₂CH(CN)—, —CH(CH₃)CH₂—, —CH₂CH(CH₃)—, —CONH— or —CON(CH₃)—; the amido, C₁₋₄ alkyl-NH₂, —Z—C₁₋₄ alkyl, —Z—C₃₋₆ monocyclic cycloalkyl, —Z-6- to 8-membered spirocycloalkyl, —Z—C₆ cycloalkenyl, —Z-phenyl, —Z-4- to 7-membered monocyclic heterocyclyl, —Z-6- to 8-membered bridged heterocyclyl, —Z-6- to 9-membered fused heterocyclyl, —Z-7- to 11-membered spiro heterocyclyl, —Z-5- to 6-membered monocyclic heteroaryl and —Z-7- to 9-membered fused heteroaryl are optionally substituted with one or more of the following substituents: hydroxyl, cyano, halogen, oxo, amido, —SO₂NH₂, optionally substituted methyl, optionally substituted ethyl, optionally substituted n-propyl, optionally substituted isopropyl, optionally substituted methoxy, optionally substituted ethoxy, optionally substituted hydroxymethyl, optionally substituted hydroxyethyl, optionally substituted phenyl, optionally substituted 5- to 6-membered heterocyclyl, optionally substituted 5- to 6-membered heteroaryl, optionally substituted —SONHCH₃, optionally substituted —SO₂CH₃, optionally substituted —COCH₃, optionally substituted —COCH₂CH₃, optionally substituted —COC₃₋₆ cycloalkyl, optionally substituted —CO-phenyl, optionally substituted —NHSO₂CH₃, and optionally substituted —CONHCH₃; the expression “optionally substituted” refers to the case of being unsubstituted or substituted with one or more of the following substituents: methyl, ethyl, hydroxyl, halogen and oxo; or,
R^{A} is selected from hydrogen, carboxyl, or the following substituents which are optionally substituted: amido, —Z-methyl, —Z-ethyl, —Z-n-propyl, —Z-isopropyl, —Z-cyclopropyl, —Z-cyclobutyl, —Z-cyclopentyl, —Z-cyclohexyl, —Z—C₆ cycloalkenyl, —Z—C₆/C₃ spirocycloalkyl, —Z-5-membered monocyclic heterocyclyl, —Z-6-membered monocyclic heterocyclyl, —Z-7-membered monocyclic heterocyclyl, —Z-5-membered monocyclic heteroaryl, —Z-6-membered monocyclic heteroaryl, —Z— phenyl, —Z-7-membered bridged heterocyclyl, —Z-8-membered bridged heterocyclyl, —Z-6-membered/3-membered fused heterocyclyl, —Z-6-membered/4-membered fused heterocyclyl, —Z-6-membered/5-membered fused heterocyclyl, —Z-3-membered/6-membered fused heterocyclyl, —Z-4-membered/6-membered fused heterocyclyl, —Z-5-membered/6-membered fused heterocyclyl, —Z-3-membered/4-membered fused heterocyclyl, —Z-4-membered/3-membered fused heterocyclyl, —Z-5-membered/3-membered fused heterocyclyl, —Z-3-membered/5-membered fused heterocyclyl, —Z-5-membered/4-membered fused heterocyclyl, —Z-4-membered/5-membered fused heterocyclyl, —Z-5-membered/5-membered fused heterocyclyl, —Z-4-membered/4-membered fused heterocyclyl, —Z-4-membered/4-membered spiro heterocyclyl, —Z-5-membered/4-membered spiro heterocyclyl, —Z-4-membered/5-membered spiro heterocyclyl, —Z-5-membered/5-membered spiro heterocyclyl, —Z-4-membered/6-membered spiro heterocyclyl, —Z-6-membered/4-membered spiro heterocyclyl, —Z-5-membered/6-membered spiro heterocyclyl, —Z-6-membered/5-membered spiro heterocyclyl, —Z-6-membered/6-membered spiro heterocyclyl, —Z-5-membered/5-membered fused heteroaryl, —Z-5-membered/6-membered fused heteroaryl, —Z-6-membered/5-membered fused heteroaryl, -methyl-NH₂, -ethyl-NH₂, —CONHCH₃ and —CONHCH₂CH₃, wherein —Z— is selected from a bond, —CH₂—, —CH₂CH₂—, —NH— or —CONH—; the expression “optionally substituted” refers to the case of being unsubstituted or substituted with one or more of the following substituents: hydroxyl, cyano, halogen, oxo, amido, —SO₂NH₂, methyl, ethyl, n-propyl, isopropyl, halomethyl, haloethyl, halo n-propyl, halo isopropyl, methoxy, ethoxy, hydroxymethyl, hydroxyethyl, phenyl, benzyl, halophenyl, 5- to 6-membered heterocyclyl, 5- to 6-membered heterocyclyl substituted with methyl, halo 5- to 6-membered heterocyclyl, 5- to 6-membered heteroaryl, 5- to 6-membered heteroaryl substituted with methyl, halo 5- to 6-membered heteroaryl, —SONHCH₃, —SO₂CH₃, —COCH₃, —COCH₂CH₃, —COC₃₋₆ cycloalkyl, —CO-phenyl, —NHSO₂CH₃ and —CONHCH₃; or, R^{A} is selected from hydrogen, carboxyl, or the following substituents which are optionally substituted: methyl, ethyl, n-propyl, isopropyl, cyclopropyl, cyclobutyl, cyclopentyl, cyclohexyl, methylamino, ethylamino, phenyl, amido, 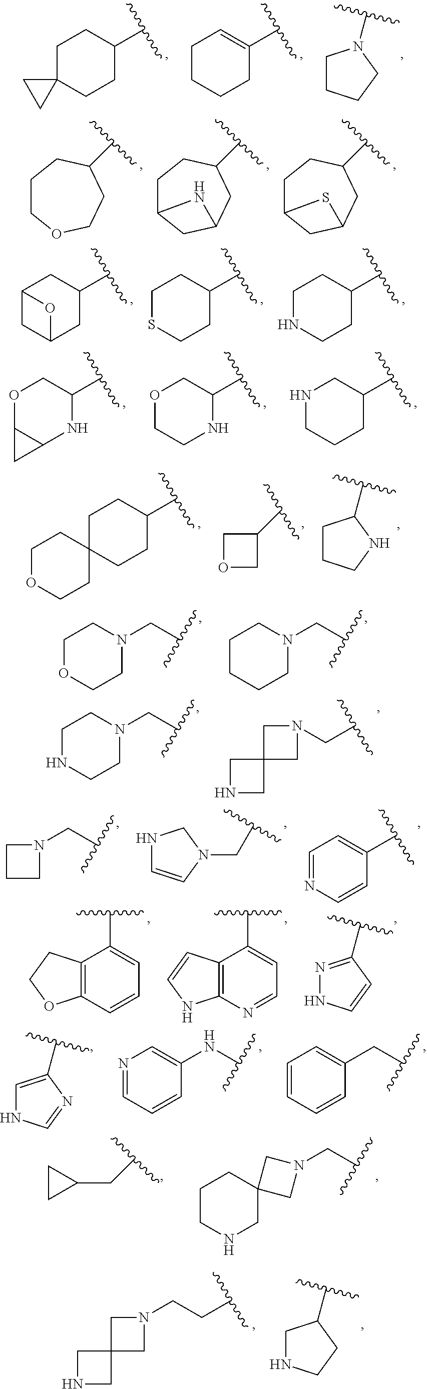 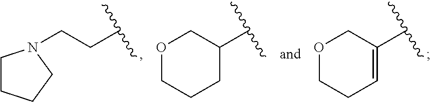
the expression optionally substituted refers to the case of being unsubstituted or substituted with one or more of the substituents selected from: hydroxyl, cyano, F, Cl, Br, oxo, amido, —SO₂NH₂, methyl, ethyl, n-propyl, isopropyl, monofluoromethyl, difluoromethyl, trifluoromethyl, methoxy, hydroxymethyl, benzyl, phenyl optionally substituted with methyl or halogen, pyridyl optionally substituted with methyl or halogen, pyrazolyl optionally substituted with methyl or halogen, —SONHCH₃, —SO₂CH₃, —COCH₃, —COCH₂CH₃, —CO-cyclopropyl, —CO— cyclobutyl, —CO-cyclopentyl, —CO-phenyl, —NHSO₂CH₃ and —CONHCH₃; or,
R^{A} is selected from one of hydrogen, carboxyl,

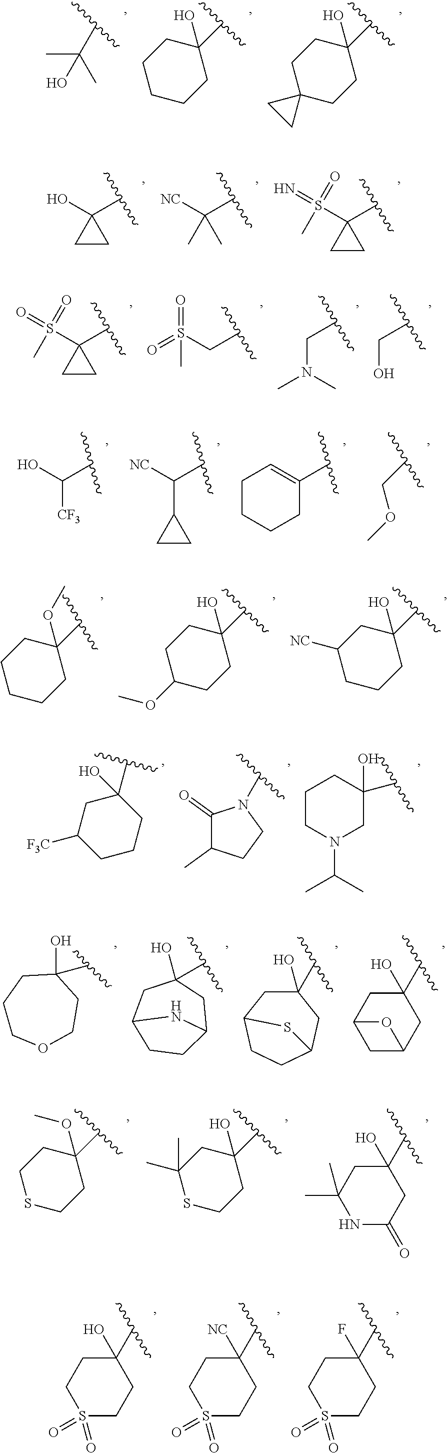

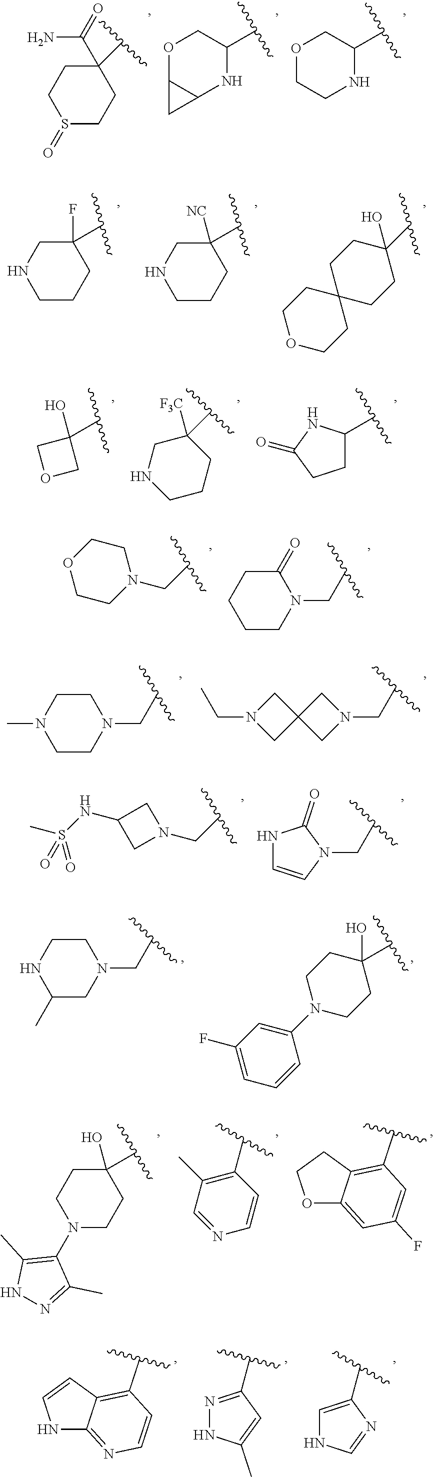

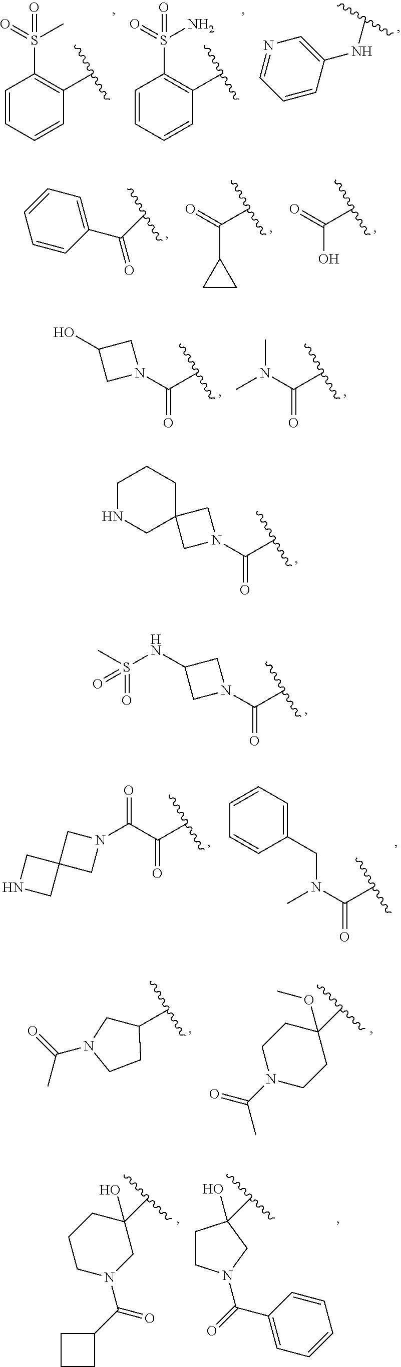

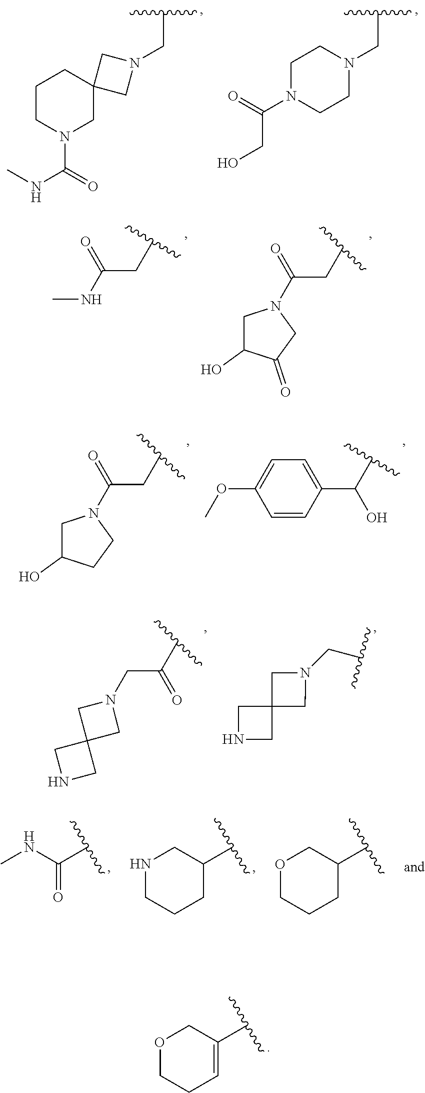

75. The compound of claim 70, which is as shown in formula (D), and a stereoisomer, an optical isomer, a pharmaceutical salt, a prodrug and a solvate thereof, 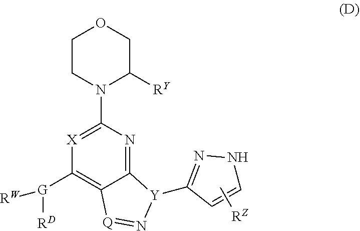
wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{Y} is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl;
G is selected from a benzene ring or a pyridine ring;
the number of R^{W} is 1, 2 or 3, and each R^{W} is independently selected from hydrogen, halogen, cyano, amino, hydroxyl, carboxyl, sulfonyl, sulfonamido, sulfone, C₁₋₆ alkyl, halo C₁₋₆ alkyl, C₁₋₆ alkoxy, C₂₋₆ alkenyl, C₂₋₆ alkynyl, C₃₋₆ cycloalkyl, C₃₋₆ cycloalkenyl, 3- to 10-membered heterocyclyl, C₆₋₁₂ aryl, 5- to 10-membered heteroaryl, —NHC₁₋₆ alkyl or —N(C₁₋₆ alkyl)₂;
R^{D} is selected from —NR⁷C(O)R⁸ or —NR⁷C(O)NR⁷R⁸, wherein each R⁷ is independently selected from hydrogen, cyano, hydroxyl, halogen, C₁₋₃ alkyl, halo C₁₋₃ alkyl, C₃₋₆ carbocyclyl or aryl, and R⁸ is selected from the following substituent which is optionally substituted: C₁₋₆ alkyl, C₃₋₁₂ carbocyclyl, C₆₋₁₂ aryl, 3- to 12-membered heterocyclyl or 5- to 12-membered heteroaryl; the expression “optionally substituted” refers to the case of being unsubstituted or substituted with one or more of the following substituents: hydroxyl, amino, cyano, halogen, oxo, C₁₋₆ alkyl, halo C₁₋₆ alkyl, —S(O)₂C₁₋₆ alkyl and —COC₁₋₆ alkyl;
the heteroatoms in the “heterocyclyl” and “heteroaryl” are selected from N, O or S, and the number of the heteroatoms is 1, 2, 3 or 4;
wherein the compound as shown in formula (D) is further represented by formula (D-1), (D-2), (D-3), (D-4) or (D-5): 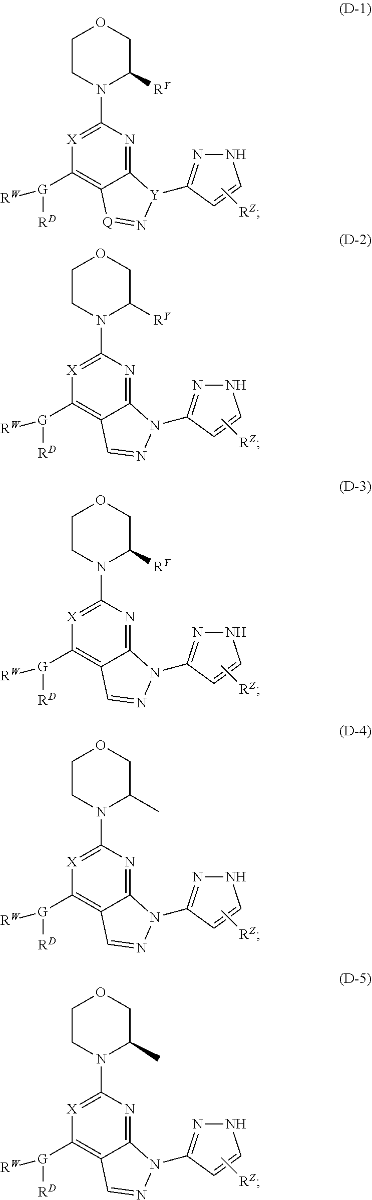
wherein the substituents in formula (D-1), (D-2), (D-3), (D-4) or (D-5) are as defined in formula (D).

76. The compound, and the stereoisomer, the optical isomer, the pharmaceutical salt, the prodrug and the solvate thereof according to claim 75, wherein Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio; or,
Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio; or,
Y is selected from N, and Q is selected from CR¹; R¹ is selected from hydrogen, halogen, cyano, amido, methyl, ethyl, methoxy, ethoxy, methylthio or ethylthio; or,
Y is selected from N, and Q is selected from CR¹; R¹ is selected from hydrogen, F, Cl, Br, cyano, amido, methyl, ethyl, methoxy or methylthio; or,
Y is selected from N, and Q is selected from CR¹; R¹ is selected from hydrogen, F, cyano, amido or methylthio; or,
Y is selected from N, and Q is selected from CR¹; R¹ is selected from hydrogen;
X is selected from CR^{X}; wherein R^{X} is selected from hydrogen, halogen, hydroxyl, sulfhydryl, amino, cyano or C₁₋₆ alkyl; or,
R^{X} is selected from hydrogen, F, Cl, Br, hydroxyl, amino, cyano or methyl; or,
R^{X} is selected from hydrogen;
R^{Y} is F, Cl, Br, methyl, ethyl, n-propyl, isopropyl or hydrogen; or,
R^{Y} is F, Cl, Br, methyl, ethyl or hydrogen; or,
R^{Y} is methyl;
the number of R^{Z} is 0, 1, 2 or 3, and R^{Z}, at each occurrence, is independently selected from F, Cl, Br, hydroxyl, cyano, amino, methyl, ethyl, monofluoromethyl, difluoromethyl or trifluoromethyl; or,
the number of R^{Z} is 0, 1 or 2, and R^{Z}, at each occurrence, is independently selected from F, Cl, Br, hydroxyl, methyl, monofluoromethyl, difluoromethyl or trifluoromethyl; or,
the number of R^{Z} is 0.

77. The compound, and the stereoisomer, the optical isomer, the pharmaceutical salt, the prodrug and the solvate thereof according to claim 75, wherein the number of R^{W} is 1, 2 or 3, and each R^{W} is independently selected from hydrogen, halogen, cyano, amino, hydroxyl, carboxyl, C₁₋₃ alkyl, halo C₁₋₃ alkyl, C₁₋₃ alkoxy, —NHC₁₋₃ alkyl or —N(C₁₋₃ alkyl)₂; or,
the number of R^{W} is 1, 2 or 3, and each R^{W} is independently selected from hydrogen, F, Cl, Br, cyano, amino, hydroxyl, carboxyl, methyl, ethyl, n-propyl, isopropyl, monofluoromethyl, difluoromethyl, trifluoromethyl, methoxy, —NHCH₃ or —N(CH₃)₂; or,
the number of R^{W} is 1, 2 or 3, and each R^{W} is independently selected from hydrogen, F, Cl, Br, cyano, amino, hydroxyl, carboxyl, methyl, monofluoromethyl, difluoromethyl, trifluoromethyl or methoxy; or,
the number of R^{W} is 1 or 2, and R^{W} is selected from hydrogen, methyl, F, cyano or methoxy;
R^{D} is selected from —NR⁷C(O)R⁸ or —NR⁷C(O)NR⁷R⁸, wherein each R⁷ is independently selected from hydrogen, cyano, hydroxyl, F, Cl, Br, methyl, ethyl, cyclopropyl or phenyl, and R⁸ is selected from the following substituent which is optionally substituted: C₁₋₄ alkyl, C₃₋₁₀ cycloalkyl, C₆₋₁₀ aryl, 3- to 8-membered heterocyclyl or 5- to 6-membered heteroaryl; the expression “optionally substituted” refers to the case of being unsubstituted or substituted with one or more of the following substituents: hydroxyl, amino, cyano, halogen, oxo, C₁₋₃ alkyl, halo C₁₋₃ alkyl, —S(O)₂C₁₋₃ alkyl and —COC₁₋₃ alkyl; or,
R^{D} is selected from —NR⁷C(O)R⁸ or —NR⁷C(O)NR⁷R⁸, wherein each R⁷ is independently selected from hydrogen, cyano, methyl, ethyl, cyclopropyl or phenyl, and R⁸ is selected from the following substituent which is optionally substituted: methyl, ethyl, n-propyl, isopropyl, C₃₋₆ monocyclic cycloalkyl, phenyl, 3- to 6-membered monocyclic heterocyclyl, 7- to 9-membered bridged heterocyclyl, C₇₋₁₀ bridged cycloalkyl or 5- to 6-membered monocyclic heteroaryl; the expression “optionally substituted” refers to the case of being unsubstituted or substituted with one or more of the following substituents: hydroxyl, amino, cyano, halogen, oxo, C₁₋₃ alkyl, halo C₁₋₃ alkyl, —S(O)₂C₁₋₃ alkyl and —COC₁₋₃ alkyl; or,
R^{D} is selected from —NR⁷C(O)R⁸ or —NR⁷C(O)NR⁷R⁸, wherein each R⁷ is independently selected from hydrogen, methyl, ethyl, cyclopropyl or phenyl, and R⁸ is selected from the following substituent which is optionally substituted: methyl, ethyl, n-propyl, isopropyl, cyclopropyl, cyclobutyl, cyclopentyl, cyclohexyl, phenyl, pyrrolidyl, tetrahydrofuryl, tetrahydropyranyl, piperidyl, pyridyl, thienyl, oxazolyl, thiazolyl, furyl, pyrazolyl, imidazolyl, pyrrolyl, piperazinyl, C₁₀ bridged cycloalkyl or 8-membered bridged heterocyclyl; the expression “optionally substituted” refers to the case of being unsubstituted or substituted with one or more of the following substituents: hydroxyl, amino, cyano, F, Cl, Br, oxo, methyl, ethyl, —S(O)₂CH₃alkyl and acetyl; or,
G is a pyridine ring, and the pyridine ring connected to R^{D} and R^{W} is selected from: 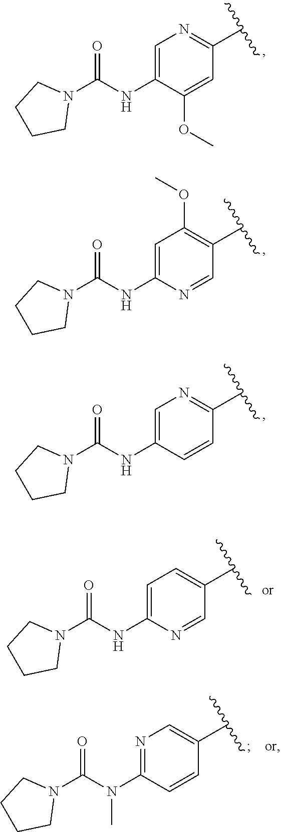 [SMILES: *c1cc(OC)c(NC(=O)N2CCCC2)cn1 ; *c1cnc(NC(=O)N2CCCC2)cc1OC ; *c1ccc(NC(=O)N2CCCC2)cn1 ; *c1ccc(NC(=O)N2CCCC2)nc1 ; *c1ccc(N(C)C(=O)N2CCCC2)nc1]
G is a benzene ring, and the benzene ring connected to R^{D} and R^{W} is selected from the structure:

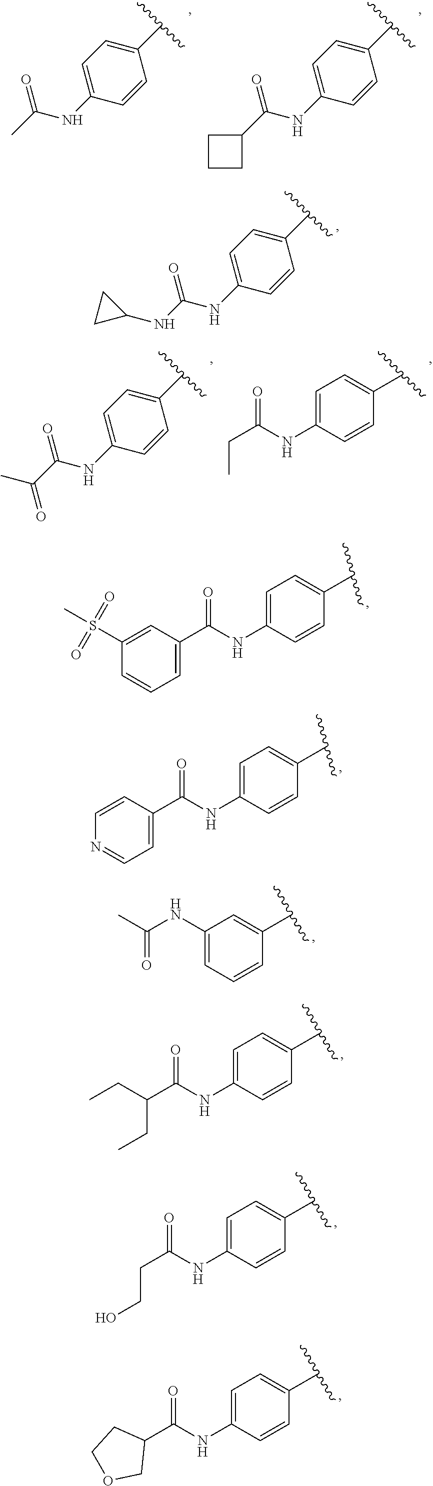

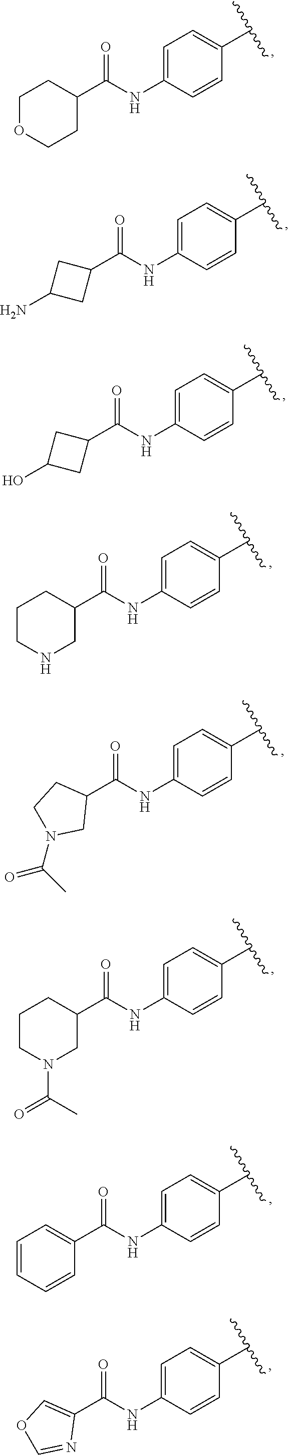

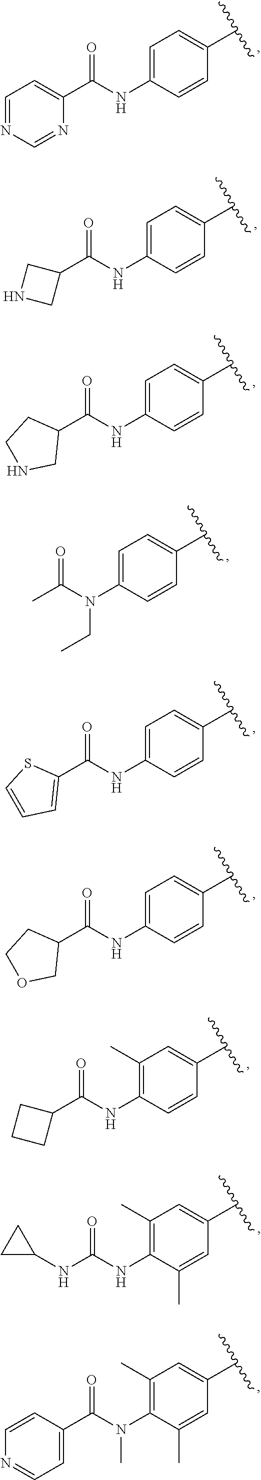

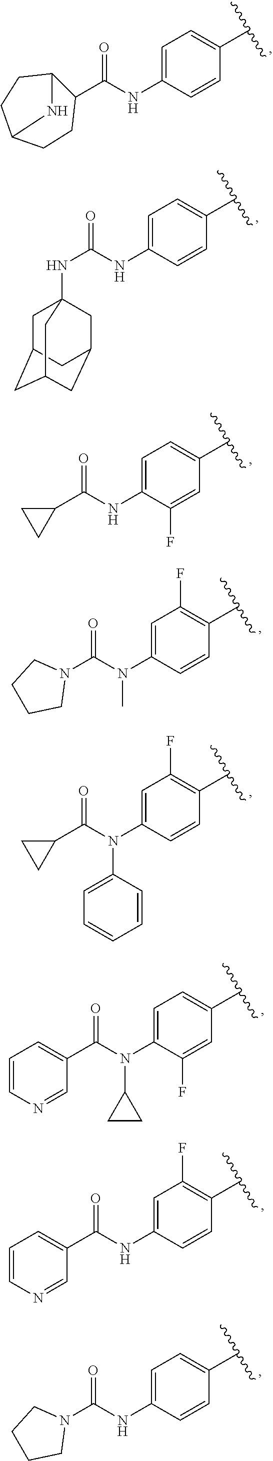

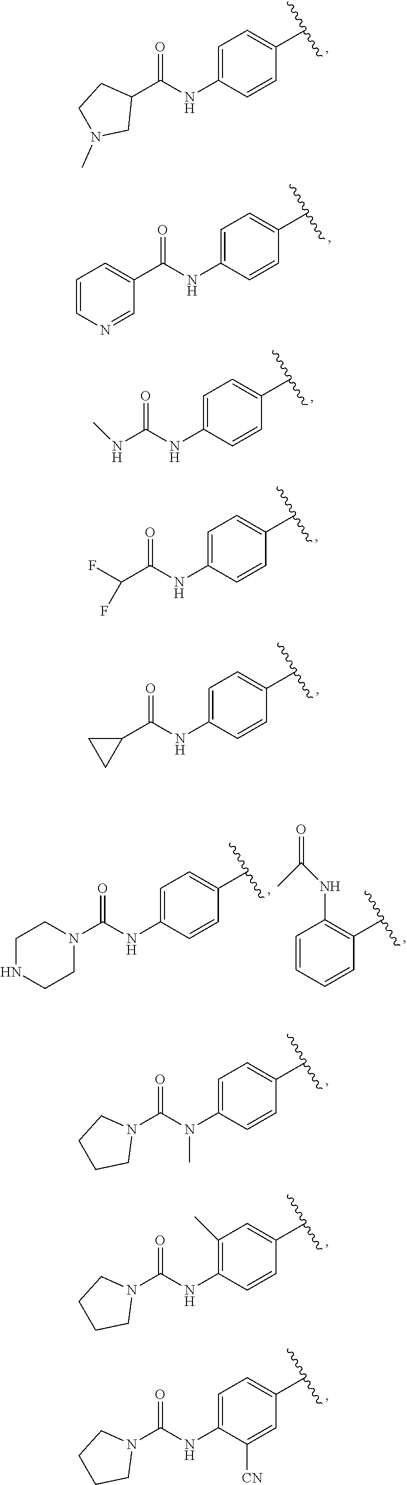

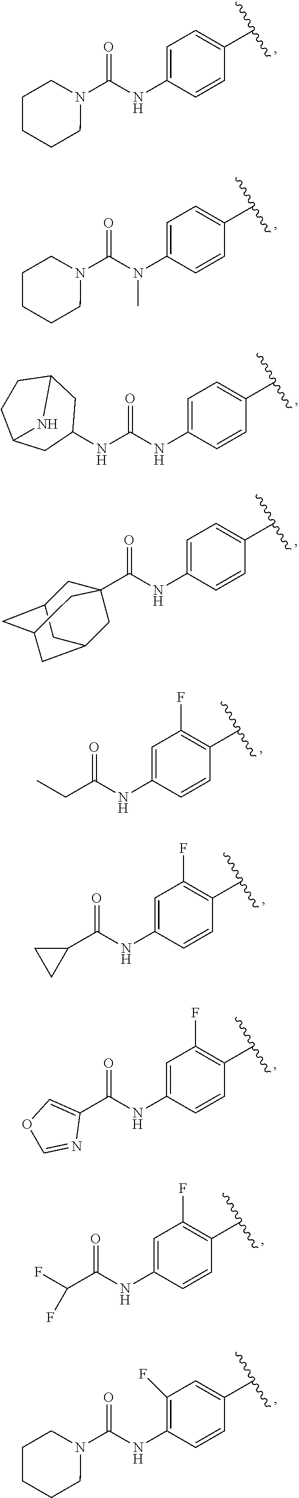

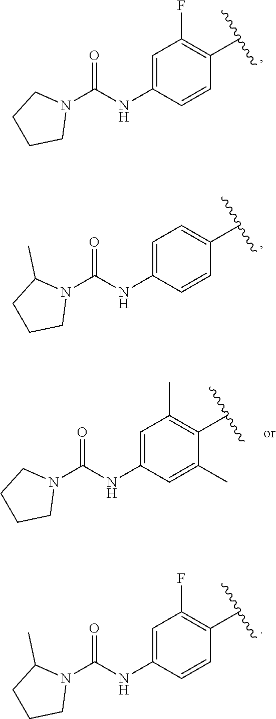 [SMILES: *c1ccc(NC(=O)N2CCCC2)cc1F ; *c1ccc(NC(=O)N2CCCC2C)cc1 ; *c1c(C)cc(NC(=O)N2CCCC2)cc1C ; *c1ccc(NC(=O)N2CCCC2C)cc1F]

78. The compound of claim 70, which is as shown in formula (E), and a stereoisomer, an optical isomer, a pharmaceutical salt, a prodrug and a solvate thereof, 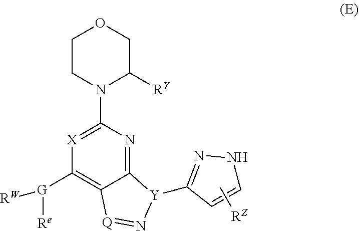
wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R¹ is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl;
G is selected from a benzene ring or a pyridine ring;
the number of R^{W} is 1, 2 or 3, and each R^{W} is independently selected from hydrogen, halogen, cyano, amino, hydroxyl, carboxyl, sulfonyl, sulfonamido, sulfone, C₁₋₆ alkyl, halo C₁₋₆ alkyl, C₁₋₆ alkoxy, C₂₋₆ alkenyl, C₂₋₆ alkynyl, C₃₋₆ cycloalkyl, C₃₋₆ cycloalkenyl, 3- to 10-membered heterocyclyl, C₆₋₁₂ aryl, 5- to 10-membered heteroaryl, —NHC₁₋₆ alkyl or —N(C₁₋₆ alkyl)₂;
R^{e} is selected from 4- to 12-membered heterocyclyl, 5- to 12-membered heteroaryl, C₃₋₁₂ carbocyclyl or C₆₋₁₂ aryl, wherein the 4- to 12-membered heterocyclyl, 5- to 12-membered heteroaryl, C₃₋₁₂ carbocyclyl and C₆₋₁₂ aryl are optionally substituted with one or more of the following substituents: halogen, hydroxyl, amino, cyano, nitro, carboxyl, oxo, C₁₋₆ alkyl, halo C₁₋₆ alkyl, C₁₋₆ alkoxy, C₁₋₆ hydroxyalkyl, C₂₋₆ alkenyl, C₂₋₆ alkynyl, C₃₋₆ cycloalkyl, C₃₋₆ cycloalkenyl, 3- to 10-membered heterocyclyl, —NHC₁₋₆ alkyl, —N(C₁₋₆ alkyl)₂, —C₁₋₆ alkyl-O—C₁₋₆ alkyl and —C₁₋₆ alkyl-C₆₋₁₂ aryl;
the heteroatoms in the “heterocyclyl” and “heteroaryl” are selected from N, O or S, and the number of the heteroatoms is 1, 2, 3 or 4;
wherein the compound as shown in formula (E) is further represented by formula (E-1), (E-2), (E-3), (E-4) or (E-5): 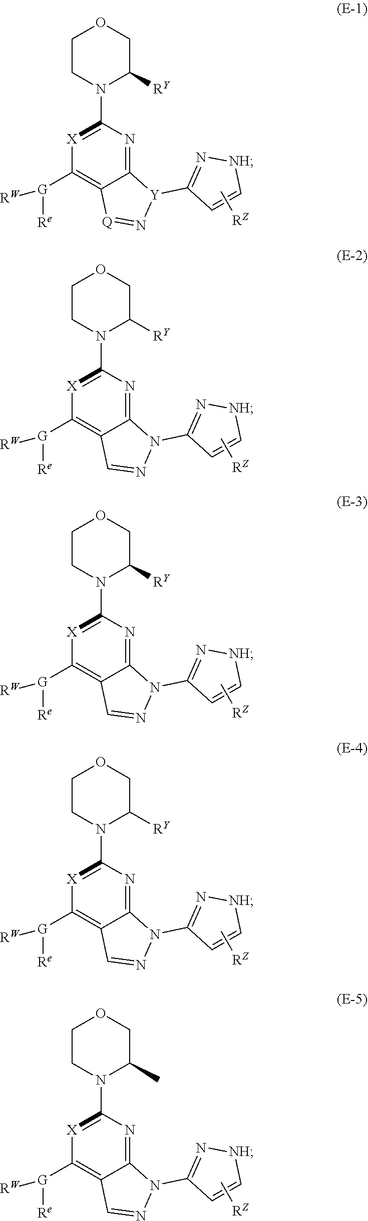
wherein the substituents in formula (E-1), (E-2), (E-3), (E-4) or (E-5) are as defined in formula (E).

79. The compound, and the stereoisomer, the optical isomer, the pharmaceutical salt, the prodrug and the solvate thereof according to claim 78, wherein Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio; or,
Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio; or,
Y is selected from N, and Q is selected from CR¹; R¹ is selected from hydrogen, halogen, cyano, amido, methyl, ethyl, methoxy, ethoxy, methylthio or ethylthio; or,
Y is selected from N, and Q is selected from CR¹; R¹ is selected from hydrogen, F, Cl, Br, cyano, amido, methyl, ethyl, methoxy or methylthio; or,
Y is selected from N, and Q is selected from CR¹; R¹ is selected from hydrogen, F, cyano, amido or methylthio; or,
Y is selected from N, and Q is selected from CR¹; R¹ is selected from hydrogen;
X is selected from CR^{X}; wherein R^{X} is selected from hydrogen, halogen, hydroxyl, sulfhydryl, amino, cyano or C₁₋₆ alkyl; or,
R^{X} is selected from hydrogen, F, Cl, Br, hydroxyl, amino, cyano or methyl; or,
R^{X} is selected from hydrogen;
R^{Y} is F, Cl, Br, methyl, ethyl, n-propyl, isopropyl or hydrogen; or
R^{Y} is F, Cl, Br, methyl, ethyl or hydrogen; or,
R^{Y} is methyl;
the number of R^{Z} is 0, 1, 2 or 3, and R^{Z}, at each occurrence, is independently selected from F, Cl, Br, hydroxyl, cyano, amino, methyl, ethyl, monofluoromethyl, difluoromethyl or trifluoromethyl; or,
the number of R^{Z} is 0, 1 or 2, and R^{Z}, at each occurrence, is independently selected from F, Cl, Br, hydroxyl, methyl, monofluoromethyl, difluoromethyl or trifluoromethyl; or,
the number of R^{Z} is 0.

80. The compound, and the stereoisomer, the optical isomer, the pharmaceutical salt, the prodrug and the solvate thereof according to claim 78, wherein the number of R^{W} is 1, 2 or 3, and each R^{W} is independently selected from hydrogen, halogen, cyano, amino, hydroxyl, carboxyl, C₁₋₃ alkyl, halo C₁₋₃ alkyl, C₁₋₃ alkoxy, —NHC₁₋₃ alkyl or —N(C₁₋₃ alkyl)₂; or,
the number of R^{W} is 1, 2 or 3, and each R^{W} is independently selected from hydrogen, F, Cl, Br, cyano, amino, hydroxyl, carboxyl, methyl, ethyl, n-propyl, isopropyl, monofluoromethyl, difluoromethyl, trifluoromethyl, methoxy, —NHCH₃ or —N(CH₃)₂; or
the number of R^{W} is 1, 2 or 3, and each R^{W} is independently selected from hydrogen, F, Cl, Br, cyano, amino, hydroxyl, carboxyl, methyl, monofluoromethyl, difluoromethyl, trifluoromethyl, methoxy or —NHCH₃; or,
the number of R^{W} is 1 or 2, and each R^{W} is independently selected from hydrogen, methyl, cyano, F, trifluoromethyl or —NHCH₃;
R^{e} is selected from 4- to 7-membered monocyclic heterocyclyl, 6- to 8-membered bridged heterocyclyl, 7- to 11-membered spiro heterocyclyl, 6- to 10-membered fused heterocyclyl, 5- to 6-membered monocyclic heteroaryl, C₅₋₆ monocyclic cycloalkyl, C₆ cycloalkenyl or phenyl, wherein the 4- to 7-membered monocyclic heterocyclyl, 6- to 8-membered bridged heterocyclyl, 7- to 11-membered spiro heterocyclyl, 8- to 10-membered fused heterocyclyl, 5- to 6-membered monocyclic heteroaryl, C₃₋₆ monocyclic cycloalkyl, C₆ cycloalkenyl and phenyl are optionally substituted with one or more of the following substituents: halogen, hydroxyl, amino, cyano, nitro, carboxyl, oxo, C₁₋₃ alkyl, halo C₁₋₃ alkyl, C₁₋₃ alkoxy, C₁₋₃ hydroxyalkyl, C₃₋₆ cycloalkyl, 3- to 6-membered monocyclic heterocyclyl, —NHC₁₋₃ alkyl, —N(C₁₋₃ alkyl)₂, C₁₋₃ alkyl-O—C₁₋₃ alkyl and —C₁₋₃ alkyl-phenyl; or,
R^{e} is selected from the following substituent which is optionally substituted: 4-membered monocyclic heterocyclyl, 5-membered monocyclic heterocyclyl, 6-membered monocyclic heterocyclyl, 7-membered monocyclic heterocyclyl, 7-membered bridged heterocyclyl, 8-membered bridged heterocyclyl, 4-membered/4-membered spiro heterocyclyl, 4-membered/5-membered spiro heterocyclyl, 5-membered/4-membered spiro heterocyclyl, 5-membered/5-membered spiro heterocyclyl, 4-membered/6-membered spiro heterocyclyl, 6-membered/4-membered spiro heterocyclyl, 5-membered/6-membered spiro heterocyclyl, 6-membered/5-membered spiro heterocyclyl, 6-membered/6-membered spiro heterocyclyl, 5-membered/3-membered fused heterocyclyl, 5-membered/5-membered fused heterocyclyl, 5-membered/6-membered fused heterocyclyl, 6-membered/5-membered fused heterocyclyl, 6-membered/6-membered fused heterocyclyl, 5-membered monocyclic heteroaryl, 6-membered monocyclic heteroaryl, cyclopentyl, cyclohexyl, C₆ cycloalkenyl or phenyl; the expression “optionally substituted” refers to the case of being unsubstituted or substituted with one or more of the following substituents: halogen, hydroxyl, amino, cyano, nitro, carboxyl, oxo, methyl, ethyl, n-propyl, isopropyl, monofluoromethyl, difluoromethyl, trifluoromethyl, methoxy, ethoxy, hydroxymethyl, hydroxyethyl, cyclopropyl, cyclobutyl, 3-membered monocyclic heterocyclyl, 4-membered monocyclic heterocyclyl, —CH₂OCH₃, —CH₂CH₂OCH₃ and —CH₂-phenyl; or,
R^{e} is selected from the following substituent which is optionally substituted: 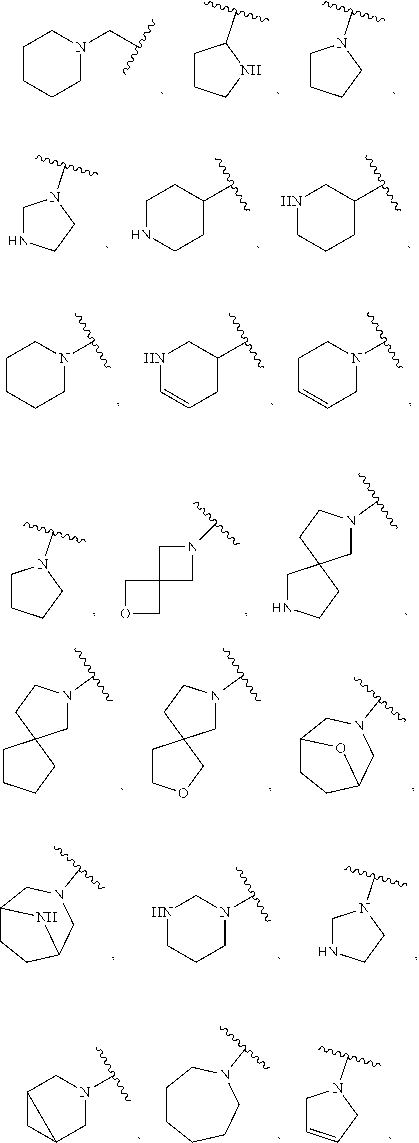 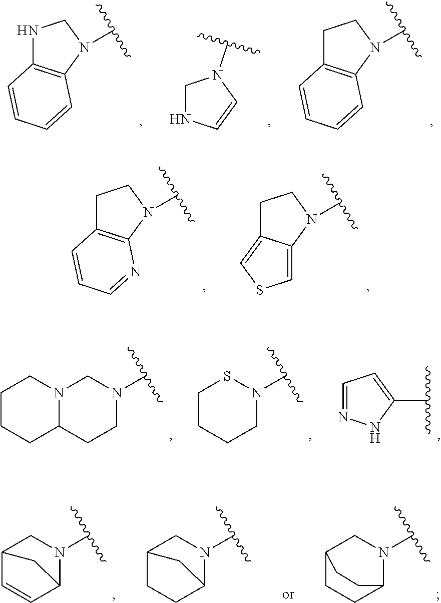
the expression “optionally substituted” refers to the case of being unsubstituted or substituted with one or more of the following substituents: F, Cl, Br, hydroxyl, cyano, oxo, methyl, ethyl, n-propyl, isopropyl, hydroxymethyl, hydroxyethyl, cyclopropyl, cyclobutyl, —CH₂OCH₃, —CH₂CH₂OCH₃ and —CH₂-phenyl.

81. The compound, and the stereoisomer, the optical isomer, the pharmaceutical salt, the prodrug and the solvate thereof according to claim 78, wherein G is a pyridine ring, and the pyridine ring connected to R^{W} and R^{e} is selected from the structure: 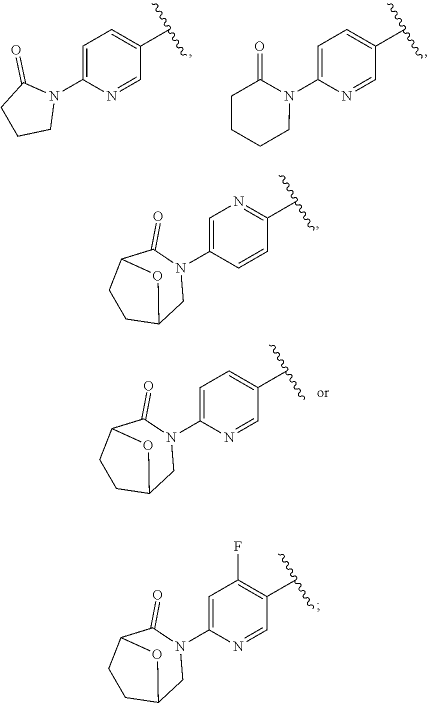 [SMILES: *c1ccc(N2CCCC2=O)nc1 ; *c1ccc(N2CCCCC2=O)nc1 ; *c1ccc(N2CC3CCC(O3)C2=O)cn1 ; *c1ccc(N2CC3CCC(O3)C2=O)nc1 ; *c1cnc(N2CC3CCC(O3)C2=O)cc1F]
or, G is a benzene ring, and the benzene ring connected to R^{W} and R^{e} is selected from the structure:

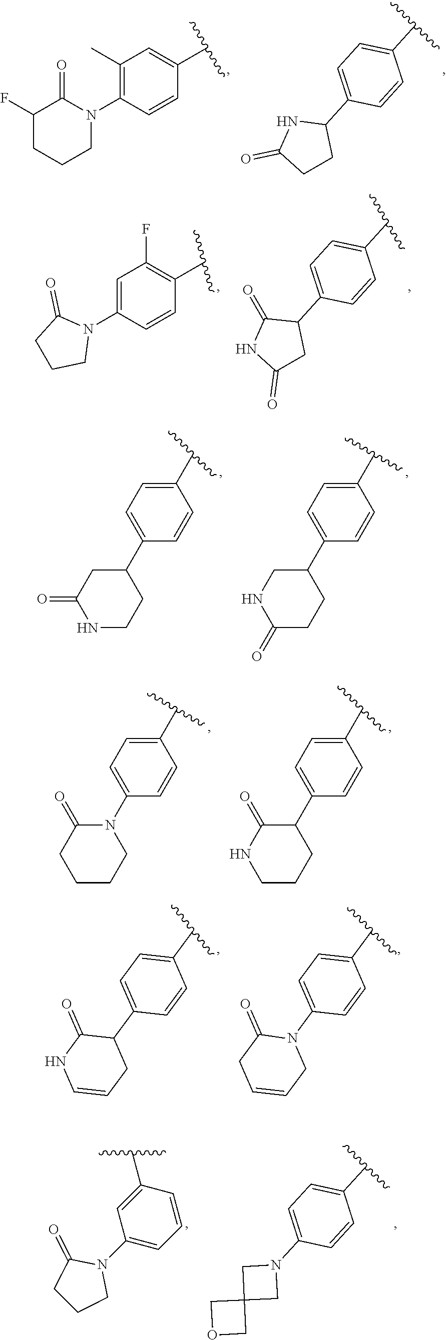

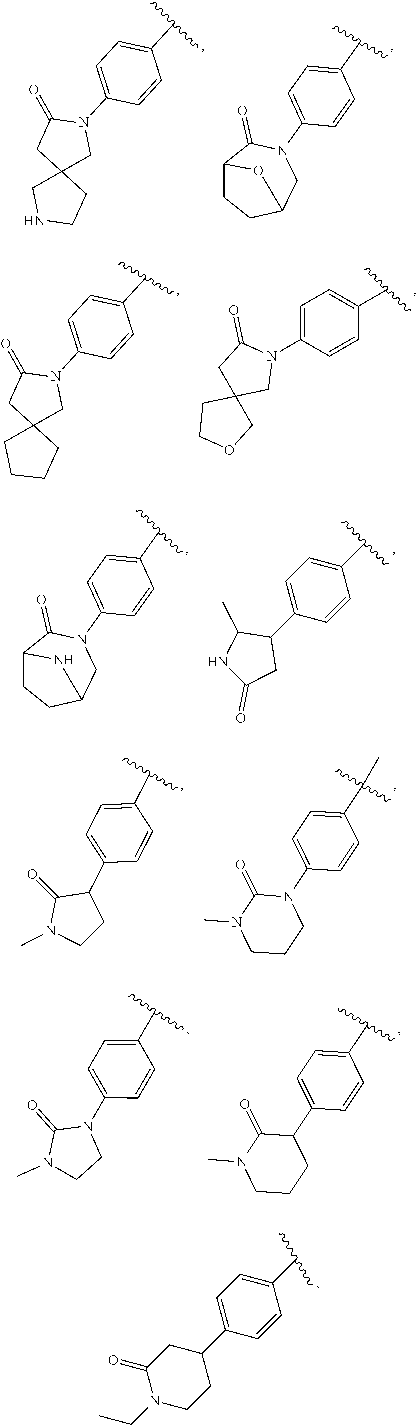

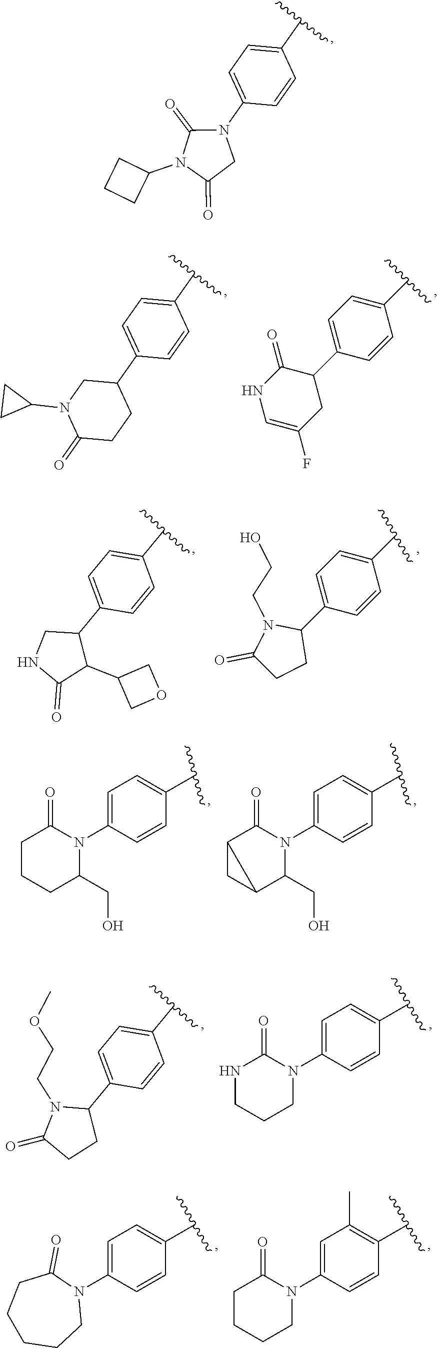

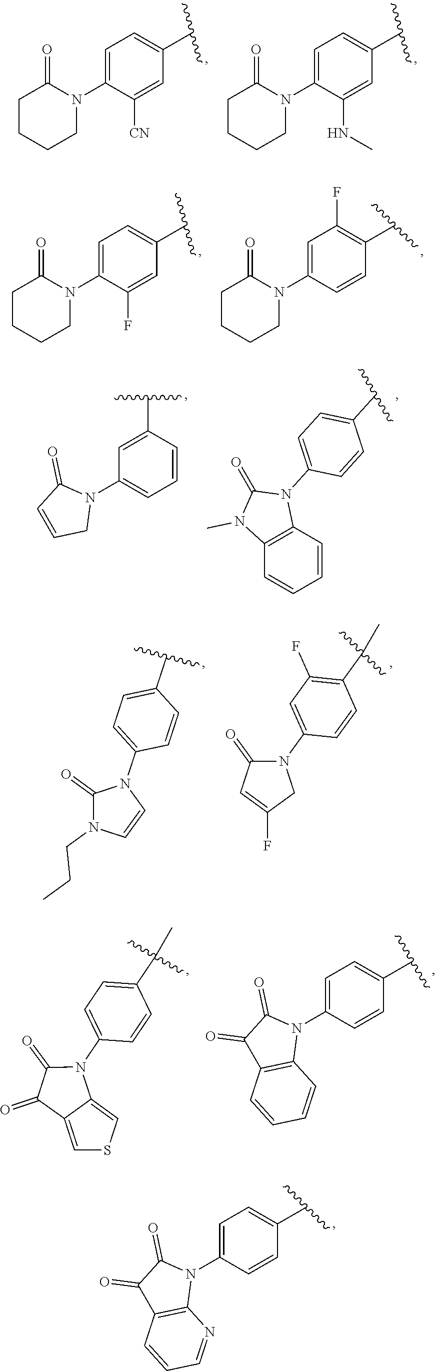

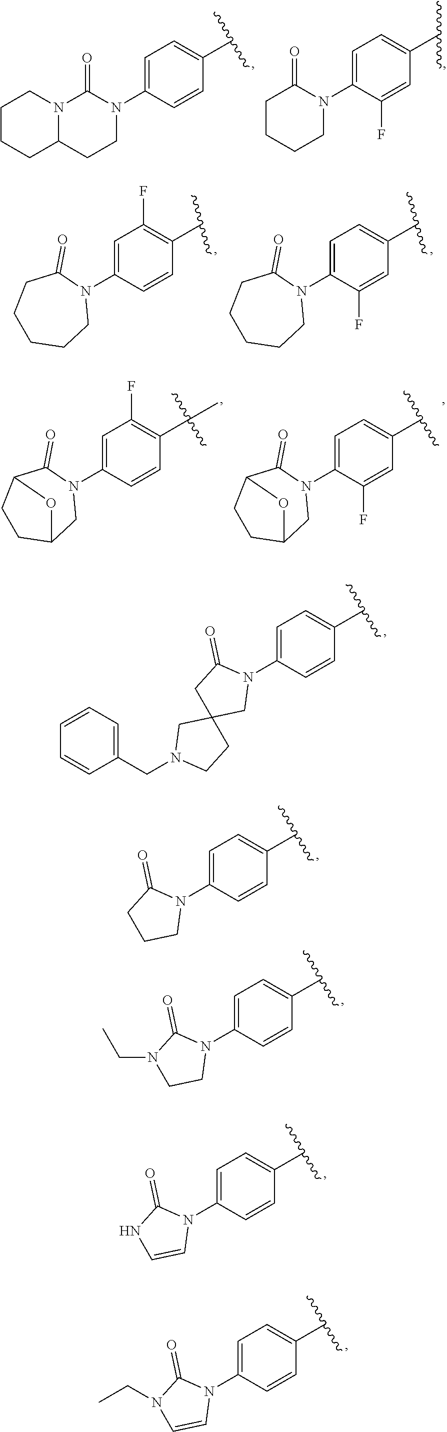

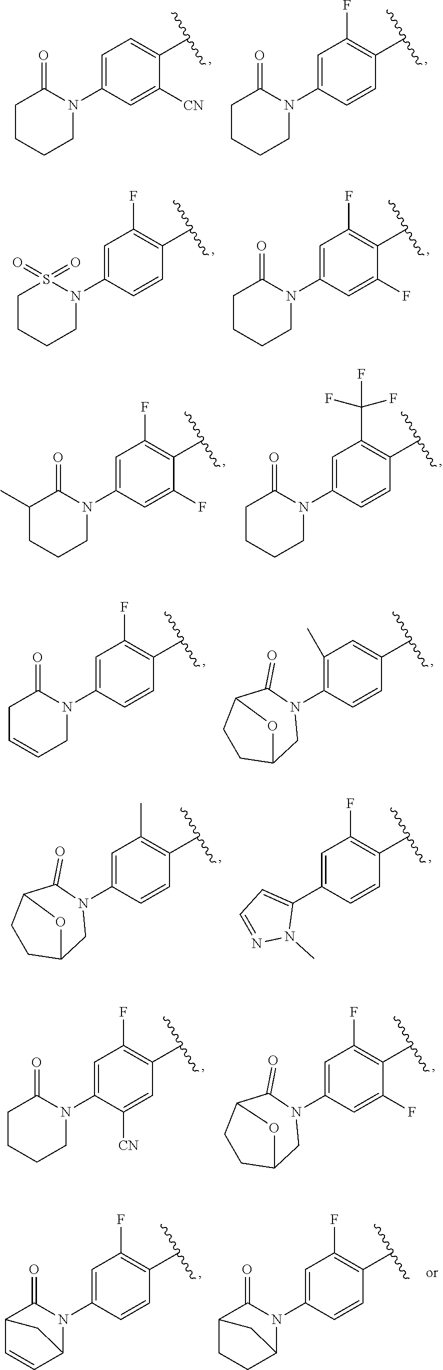

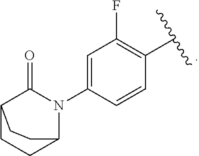 [SMILES: *c1ccc(N2C(=O)C3CCC2CC3)cc1F]

82. The compound of claim 70, which is as shown in formula (F), and a stereoisomer, an optical isomer, a pharmaceutical salt, a prodrug and a solvate thereof, 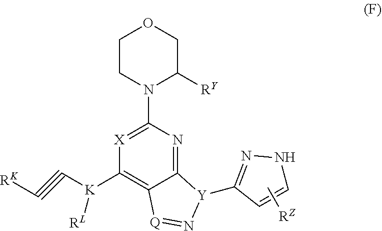
wherein Q and Y are each selected from CR¹ or N; R¹ is selected from hydrogen, halogen, hydroxyl, sulfhydryl, amino, cyano, amido, C₁₋₆ alkyl, C₂₋₆ alkenyl, C₂₋₆ alkynyl, C₃₋₆ cycloalkyl, C₁₋₆ alkoxy, C₁₋₆ alkylthio, halo C₁₋₆ alkyl, halo C₁₋₆ alkoxy or 4- to 6-membered heterocyclyl;
X is selected from CR^{X} or N; wherein R^{X}, at each occurrence, is independently selected from hydrogen, halogen, hydroxyl, sulfhydryl, amino, cyano, C₁₋₆ alkyl, C₂₋₆ alkenyl, C₁₋₆ alkoxy, C₃₋₈ cycloalkyl or halo C₁₋₆ alkyl;
R^{Y} is halogen, C₁₋₆ alkyl or hydrogen;
the number of R^{Z} is 0, 1, 2 or 3, and R^{Z}, at each occurrence, is independently selected from halogen, hydroxyl, cyano, amino, C₁₋₃ alkyl or halo C₁₋₃ alkyl;
K is selected from C₅₋₆ carbocyclyl or 5- to 7-membered heterocyclyl;
the number of R^{L} is 1 or 2, and R^{L}, at each occurrence, is independently selected from hydrogen, halogen, hydroxyl, cyano or C₁₋₃ alkyl;
R^{K} is selected from hydrogen, C₁₋₃ alkyl, —C(O) C₁₋₃ alkyl, halo C₁₋₃ alkyl, C₃₋₆ cycloalkyl, 5- to 6-membered heteroaryl, 5- to 8-membered heterocyclyl or phenyl, wherein the C₁₋₃ alkyl, —C(O) C₁-3 alkyl, halo C₁₋₃ alkyl, C₃₋₆ cycloalkyl, 5- to 6-membered heteroaryl, 5- to 8-membered heterocyclyl and phenyl are optionally substituted with substituents selected from hydroxyl, C₁₋₃ alkyl and halogen;
the heteroatoms in the “heterocyclyl” and “heteroaryl” are selected from N, O or S, and the number of the heteroatoms is 1, 2, 3 or 4;
wherein the compound as shown in formula (F) is further represented by formula (F-1), (F-2), (F-3), (F-4) or (F-5): 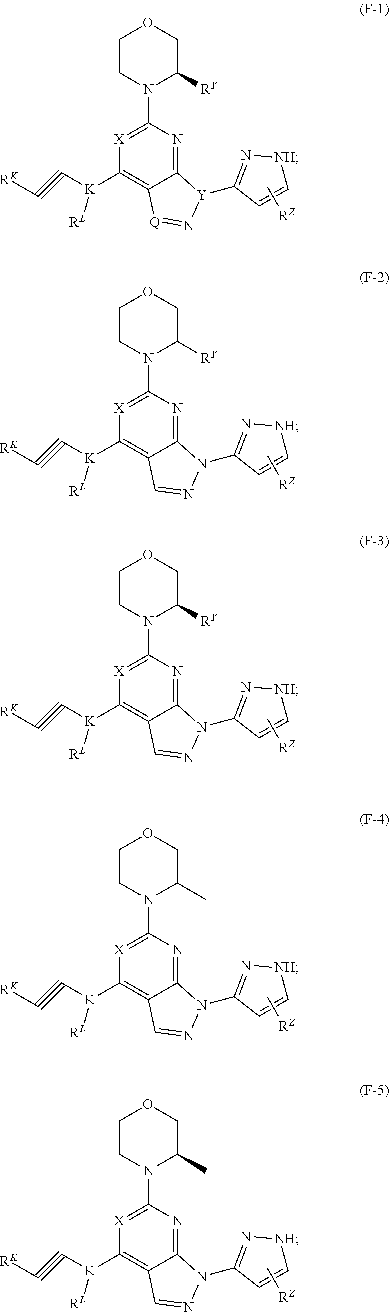
wherein the substituents in formula (F-1), (F-2), (F-3), (F-4) or (F-5) are as defined in formula (F).

83. The compound, and the stereoisomer, the optical isomer, the pharmaceutical salt, the prodrug and the solvate thereof according to claim 82, wherein Y is selected from N, and Q is selected from CR¹; R¹ is selected from hydrogen, halogen, hydroxyl, sulfhydryl, amino, cyano, amido, C₁₋₆ alkyl, C₁₋₆ alkoxy or C₁₋₆ alkylthio; or,
Y is selected from N, and Q is selected from CR¹; R¹ is selected from hydrogen, halogen, hydroxyl, sulfhydryl, amino, cyano, amido, methyl, ethyl, n-propyl, isopropyl, methoxy, ethoxy, methylthio or ethylthio; or,
Y is selected from N, and Q is selected from CR¹; R¹ is selected from hydrogen, halogen, cyano, amido, methyl, ethyl, methoxy, ethoxy, methylthio or ethylthio; or,
Y is selected from N, and Q is selected from CR¹; R¹ is selected from hydrogen, F, Cl, Br, cyano, amido, methyl, ethyl, methoxy or methylthio; or,
Y is selected from N, and Q is selected from CR¹; R¹ is selected from hydrogen, F, cyano, amido or methylthio; or,
Y is selected from N, and Q is selected from CR¹; R¹ is selected from hydrogen;
X is selected from CR^{X}; wherein R^{X} is selected from hydrogen, halogen, hydroxyl, sulfhydryl, amino, cyano or C₁₋₆ alkyl; or,
R^{X} is selected from hydrogen, F, Cl, Br, hydroxyl, amino, cyano or methyl; or,
R^{X} is selected from hydrogen;
R^{Y} is F, Cl, Br, methyl, ethyl, n-propyl, isopropyl or hydrogen; or,
R^{Y} is F, Cl, Br, methyl, ethyl or hydrogen; or,
R^{Y} is methyl;
the number of R^{Z} is 0, 1, 2 or 3, and R^{Z}, at each occurrence, is independently selected from F, Cl, Br, hydroxyl, cyano, amino, methyl, ethyl, monofluoromethyl, difluoromethyl or trifluoromethyl; or,
the number of R^{Z} is 0, 1 or 2, and R^{Z}, at each occurrence, is independently selected from F, Cl, Br, hydroxyl, methyl, monofluoromethyl, difluoromethyl or trifluoromethyl; or,
the number of R^{Z} is 0.

84. The compound, and the stereoisomer, the optical isomer, the pharmaceutical salt, the prodrug and the solvate thereof according to claim 82, wherein K is selected from cyclopentyl, cyclohexyl, 5-membered monocyclic heterocycloalkyl, 6-membered monocyclic heterocycloalkyl, 7-membered bridged heterocyclyl or cyclohexenyl; or,
K is selected from cyclopentyl, cyclohexyl, 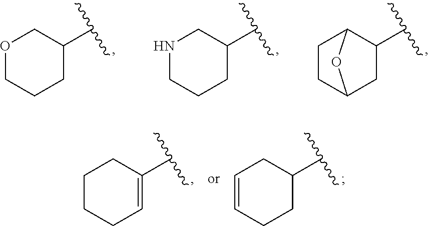 [SMILES: *C1CCCOC1 ; *C1CCCNC1 ; *C1CC2CCC1O2 ; *C1=CCCCC1 ; *C1CC=CCC1]
or,
K is selected from cyclopentyl, cyclohexyl or 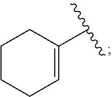 [SMILES: *C1=CCCCC1]
the number of R^{L} is 1 or 2, and R^{L} is selected from hydrogen, halogen, hydroxyl, cyano or methyl; or,
the number of R^{L} is 1, and R^{L} is selected from hydrogen, hydroxyl or methyl; or,
the number of R^{L} is 1, and R^{L} is selected from hydrogen or hydroxyl;
R^{K} is selected from hydrogen, methyl, ethyl, n-propyl, isopropyl, —C(O) CH₃, —C(O) CH₂CH₃, C₃₋₆ monocyclic cycloalkyl, 5- to 6-membered monocyclic heteroaryl, 5- to 6-membered monocyclic heterocyclyl, 6- to 8-membered spiro heterocyclyl or phenyl, wherein the methyl, ethyl, n-propyl, isopropyl, —C(O) CH₃, —C(O) CH₂CH₃, C₃₋₆ monocyclic cycloalkyl, 5- to 6-membered monocyclic heteroaryl, 5- to 6-membered monocyclic heterocyclyl, 6- to 8-membered spiro heterocyclyl and phenyl are optionally substituted with substituents selected from hydroxyl, methyl, ethyl and halogen; or,
R^{K} is selected from hydrogen, methyl, ethyl, —C(O) CH₃, cyclopropyl, cyclobutyl, cyclopentyl, 5- to 6-membered monocyclic heteroaryl, 5- to 6-membered monocyclic heterocyclyl, 7-membered spiro heterocyclyl or phenyl, wherein the methyl, ethyl, —C(O) CH₃, cyclopropyl, cyclobutyl, cyclopentyl, 5- to 6-membered monocyclic heteroaryl, 5- to 6-membered monocyclic heterocyclyl, 7-membered spiro heterocyclyl and phenyl are optionally substituted with substituents selected from hydroxyl, methyl, ethyl and halogen; or,
R^{K} is selected from hydrogen, methyl, ethyl, —C(O) CH₃, cyclopropyl, cyclobutyl, imidazolyl, pyrazolyl, tetrahydrofuryl, 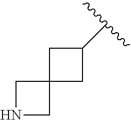 [SMILES: *C1CC2(CNC2)C1]
or phenyl, wherein the methyl, ethyl, —C(O) CH₃, cyclopropyl, imidazolyl, pyrazolyl, tetrahydrofuryl, 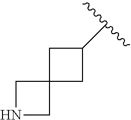 [SMILES: *C1CC2(CNC2)C1]
and phenyl are optionally substituted with substituents selected from hydroxyl, methyl, F, Cl and Br; or,
R^{K} is selected from hydrogen, methyl, hydroxymethyl, —CF₂CH₃, —C(O) CH₃, cyclopropyl, 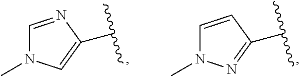
tetrahydrofuryl, 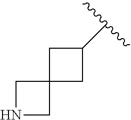 [SMILES: *C1CC2(CNC2)C1]
or phenyl; or,
R^{K} is selected from hydrogen.

85. The compound, and the stereoisomer, the optical isomer, the pharmaceutical salt, the prodrug and the solvate thereof according to claim 82, wherein 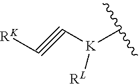
is selected from the following structure:

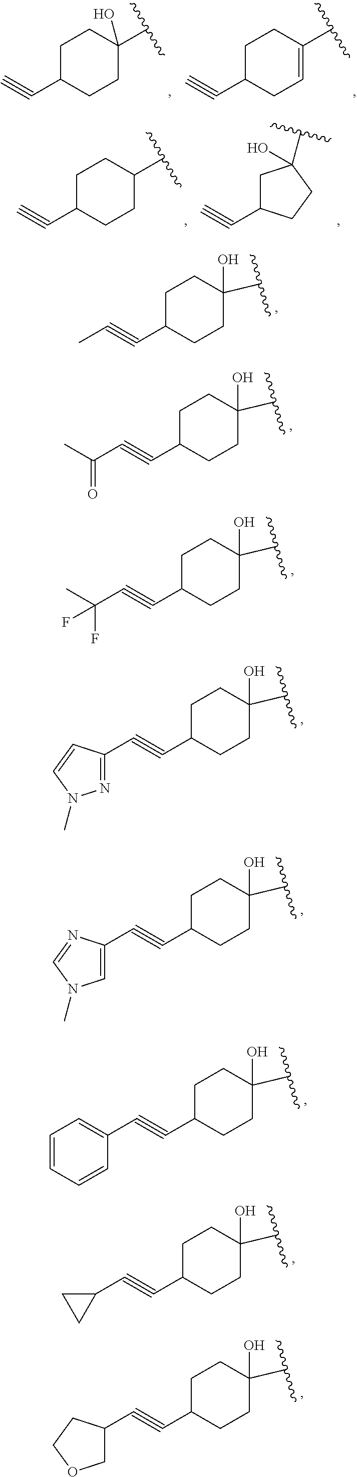

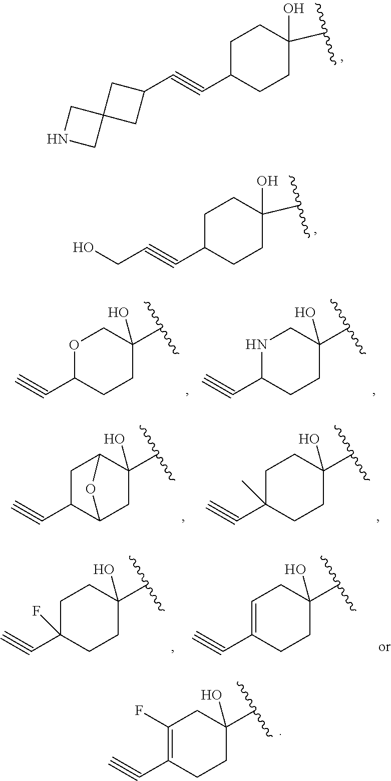

86. The compound of claim 70, and a stereoisomer, an optical isomer, a pharmaceutical salt, a prodrug and a solvate thereof, wherein the compound is selected from:

| No. | Structure | No. | Structure |
|---|---|---|---|
| 1 | 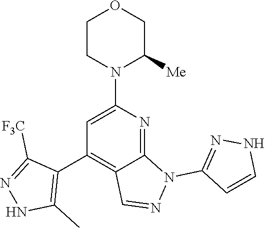 [SMILES: Cc1[nH]nc(C(F)(F)F)c1-c1cc(N2CCOC[C@H]2C)nc2c1cnn2-c1cc[nH]n1] | 2 | 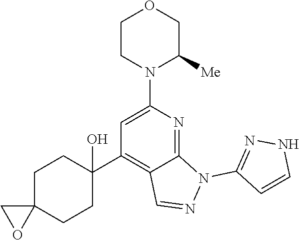 [SMILES: C[C@@H]1COCCN1c1cc(C2(O)CCC3(CC2)CO3)c2cnn(-c3cc[nH]n3)c2n1] |
| 3 | 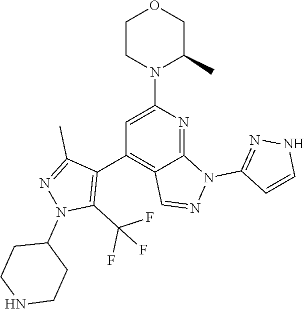 [SMILES: Cc1nn(C2CCNCC2)c(C(F)(F)F)c1-c1cc(N2CCOC[C@H]2C)nc2c1cnn2-c1cc[nH]n1] | 4 | 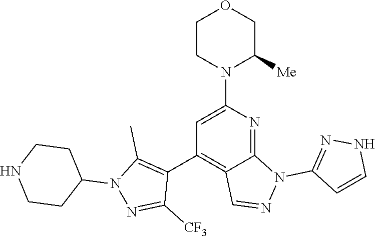 [SMILES: Cc1c(-c2cc(N3CCOC[C@H]3C)nc3c2cnn3-c2cc[nH]n2)c(C(F)(F)F)nn1C1CCNCC1] |
| 5 | 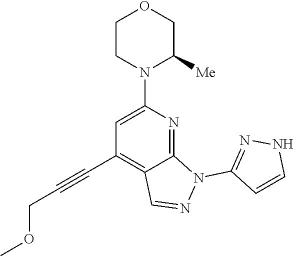 [SMILES: COCC#Cc1cc(N2CCOC[C@H]2C)nc2c1cnn2-c1cc[nH]n1] | 6 | 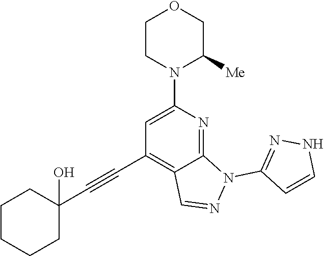 |
| 7 | 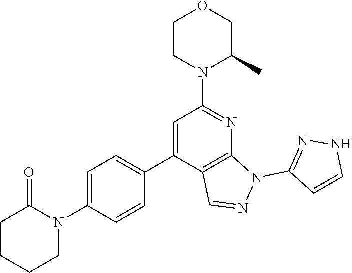 [SMILES: C[C@@H]1COCCN1c1cc(-c2ccc(N3CCCCC3=O)cc2)c2cnn(-c3cc[nH]n3)c2n1] | 8 | 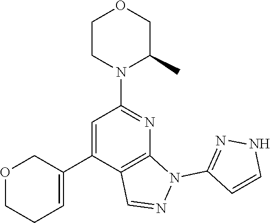 [SMILES: C[C@@H]1COCCN1c1cc(C2=CCCOC2)c2cnn(-c3cc[nH]n3)c2n1] |
| 9 | 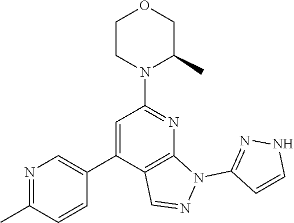 [SMILES: Cc1ccc(-c2cc(N3CCOC[C@H]3C)nc3c2cnn3-c2cc[nH]n2)cn1] | 10 | 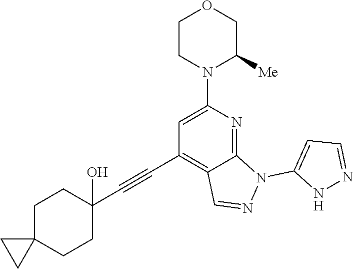 [SMILES: C[C@@H]1COCCN1c1cc(C#CC2(O)CCC3(CC2)CC3)c2cnn(-c3ccn[nH]3)c2n1] |
| 11 | 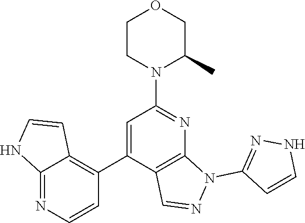 [SMILES: C[C@@H]1COCCN1c1cc(-c2ccnc3[nH]ccc23)c2cnn(-c3cc[nH]n3)c2n1] | 12 | 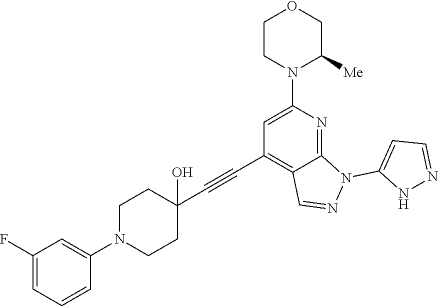 [SMILES: C[C@@H]1COCCN1c1cc(C#CC2(O)CCN(c3cccc(F)c3)CC2)c2cnn(-c3ccn[nH]3)c2n1] |
| 13 | 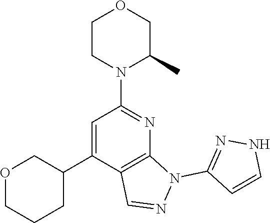 [SMILES: C[C@@H]1COCCN1c1cc(C2CCCOC2)c2cnn(-c3cc[nH]n3)c2n1] | 14 | 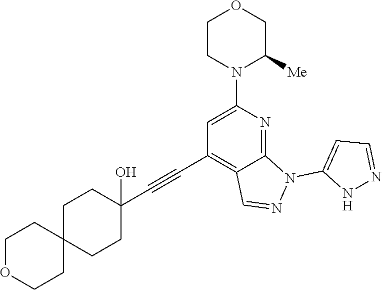 [SMILES: C[C@@H]1COCCN1c1cc(C#CC2(O)CCC3(CCOCC3)CC2)c2cnn(-c3ccn[nH]3)c2n1] |
| 15 | 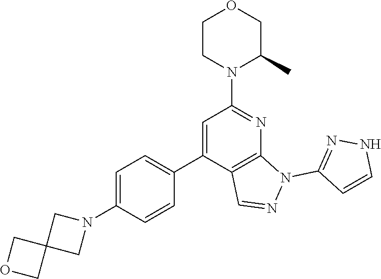 [SMILES: C[C@@H]1COCCN1c1cc(-c2ccc(N3CC4(COC4)C3)cc2)c2cnn(-c3cc[nH]n3)c2n1] | 16 | 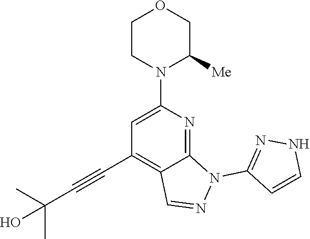 [SMILES: C[C@@H]1COCCN1c1cc(C#CC(C)(C)O)c2cnn(-c3cc[nH]n3)c2n1] |
| 17 | 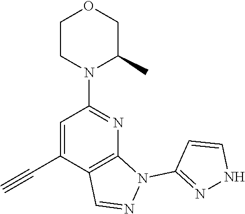 [SMILES: C#Cc1cc(N2CCOC[C@H]2C)nc2c1cnn2-c1cc[nH]n1] | 18 | 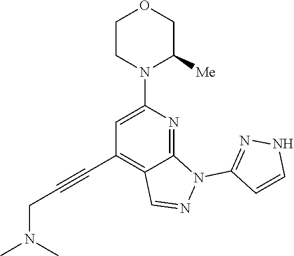 [SMILES: C[C@@H]1COCCN1c1cc(C#CCN(C)C)c2cnn(-c3cc[nH]n3)c2n1] |
| 19 | 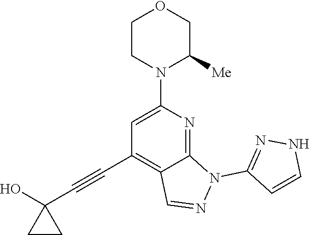 [SMILES: C[C@@H]1COCCN1c1cc(C#CC2(O)CC2)c2cnn(-c3cc[nH]n3)c2n1] | 20 | 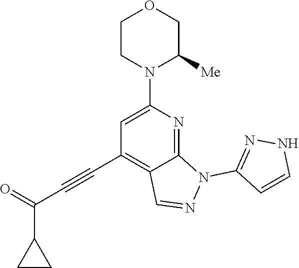 [SMILES: C[C@@H]1COCCN1c1cc(C#CC(=O)C2CC2)c2cnn(-c3cc[nH]n3)c2n1] |
| 21 | 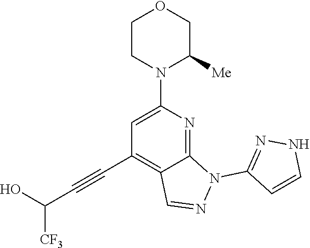 [SMILES: C[C@@H]1COCCN1c1cc(C#CC(O)C(F)(F)F)c2cnn(-c3cc[nH]n3)c2n1] | 22 | 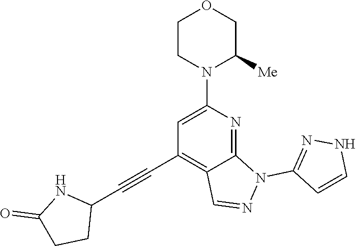 [SMILES: C[C@@H]1COCCN1c1cc(C#CC2CCC(=O)N2)c2cnn(-c3cc[nH]n3)c2n1] |
| 23 | 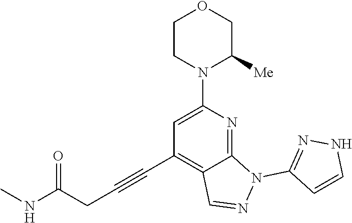 [SMILES: CNC(=O)CC#Cc1cc(N2CCOC[C@H]2C)nc2c1cnn2-c1cc[nH]n1] | 24 | 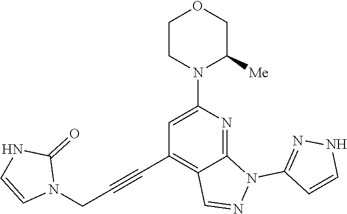 [SMILES: C[C@@H]1COCCN1c1cc(C#CCn2cc[nH]c2=O)c2cnn(-c3cc[nH]n3)c2n1] |
| 25 | 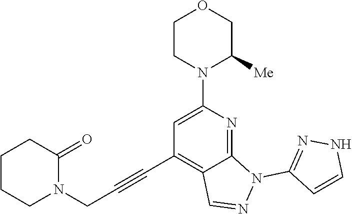 [SMILES: C[C@@H]1COCCN1c1cc(C#CCN2CCCCC2=O)c2cnn(-c3cc[nH]n3)c2n1] | 26 | 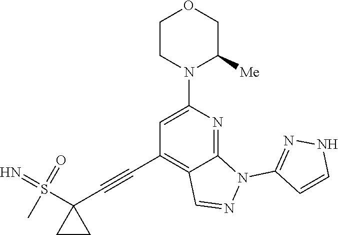 [SMILES: C[C@@H]1COCCN1c1cc(C#CC2(S(C)(=N)=O)CC2)c2cnn(-c3cc[nH]n3)c2n1] |
| 27 | 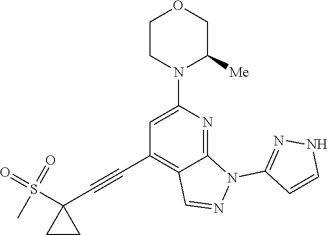 [SMILES: C[C@@H]1COCCN1c1cc(C#CC2(S(C)(=O)=O)CC2)c2cnn(-c3cc[nH]n3)c2n1] | 28 | 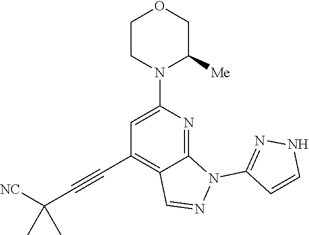 [SMILES: C[C@@H]1COCCN1c1cc(C#CC(C)(C)C#N)c2cnn(-c3cc[nH]n3)c2n1] |
| 29 | 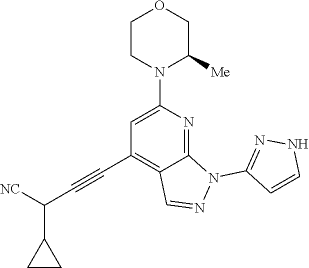 [SMILES: C[C@@H]1COCCN1c1cc(C#CC(C#N)C2CC2)c2cnn(-c3cc[nH]n3)c2n1] | 30 | 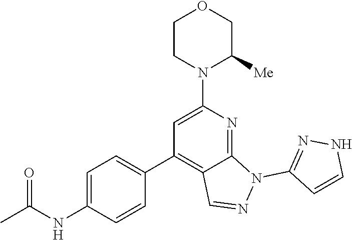 [SMILES: CC(=O)Nc1ccc(-c2cc(N3CCOC[C@H]3C)nc3c2cnn3-c2cc[nH]n2)cc1] |
| 31 | 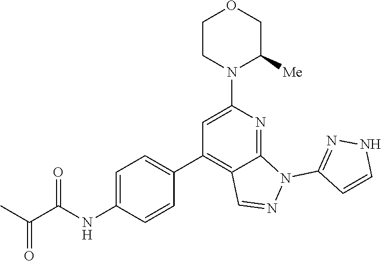 [SMILES: CC(=O)C(=O)Nc1ccc(-c2cc(N3CCOC[C@H]3C)nc3c2cnn3-c2cc[nH]n2)cc1] | 32 | 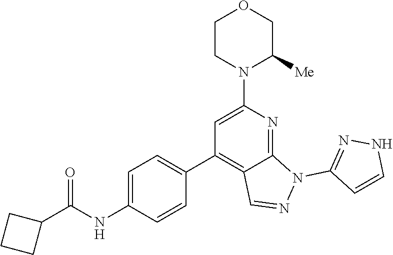 [SMILES: C[C@@H]1COCCN1c1cc(-c2ccc(NC(=O)C3CCC3)cc2)c2cnn(-c3cc[nH]n3)c2n1] |
| 33 | 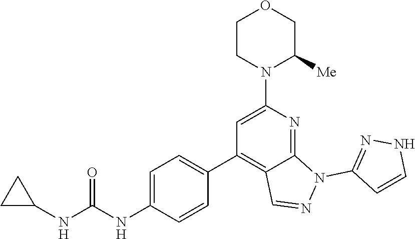 [SMILES: C[C@@H]1COCCN1c1cc(-c2ccc(NC(=O)NC3CC3)cc2)c2cnn(-c3cc[nH]n3)c2n1] | 34 | 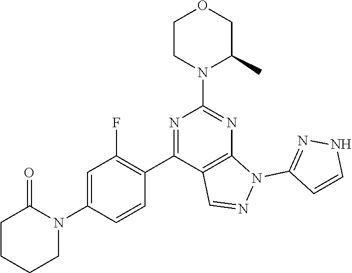 [SMILES: C[C@@H]1COCCN1c1nc(-c2ccc(N3CCCCC3=O)cc2F)c2cnn(-c3cc[nH]n3)c2n1] |
| 35 | 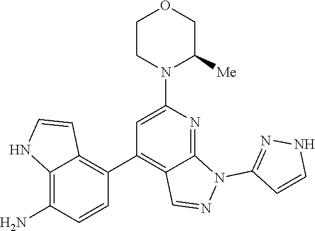 [SMILES: C[C@@H]1COCCN1c1cc(-c2ccc(N)c3[nH]ccc23)c2cnn(-c3cc[nH]n3)c2n1] | 36 | 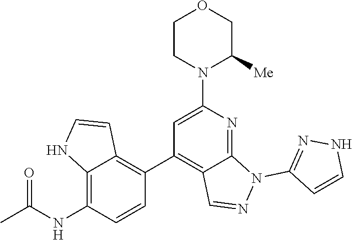 [SMILES: CC(=O)Nc1ccc(-c2cc(N3CCOC[C@H]3C)nc3c2cnn3-c2cc[nH]n2)c2cc[nH]c12] |
| 37 | 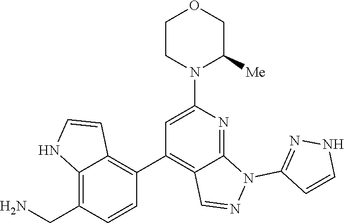 [SMILES: C[C@@H]1COCCN1c1cc(-c2ccc(CN)c3[nH]ccc23)c2cnn(-c3cc[nH]n3)c2n1] | 38 | 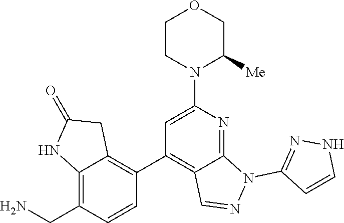 [SMILES: C[C@@H]1COCCN1c1cc(-c2ccc(CN)c3c2CC(=O)N3)c2cnn(-c3cc[nH]n3)c2n1] |
| 39 | 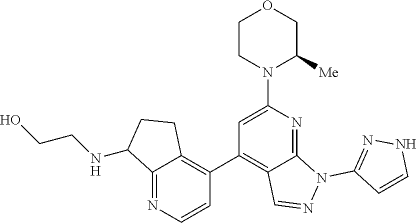 [SMILES: C[C@@H]1COCCN1c1cc(-c2ccnc3c2CCC3NCCO)c2cnn(-c3cc[nH]n3)c2n1] | 40 | 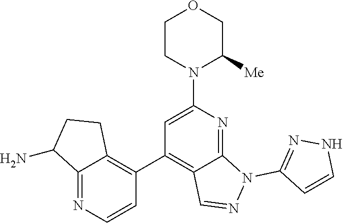 [SMILES: C[C@@H]1COCCN1c1cc(-c2ccnc3c2CCC3N)c2cnn(-c3cc[nH]n3)c2n1] |
| 41 | 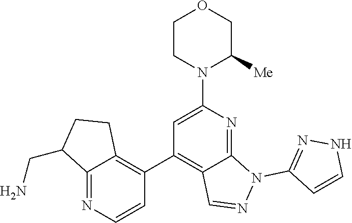 [SMILES: C[C@@H]1COCCN1c1cc(-c2ccnc3c2CCC3CN)c2cnn(-c3cc[nH]n3)c2n1] | 42 | 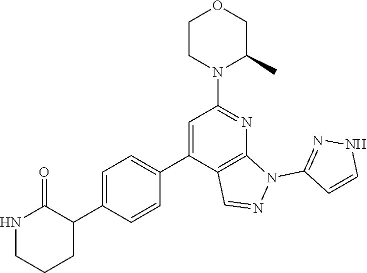 [SMILES: C[C@@H]1COCCN1c1cc(-c2ccc(C3CCCNC3=O)cc2)c2cnn(-c3cc[nH]n3)c2n1] |
| 43 | 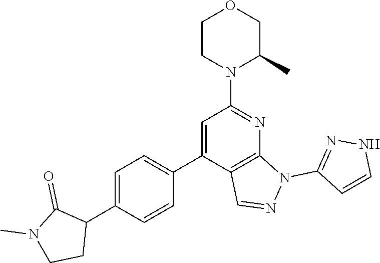 [SMILES: C[C@@H]1COCCN1c1cc(-c2ccc(C3CCN(C)C3=O)cc2)c2cnn(-c3cc[nH]n3)c2n1] | 44 | 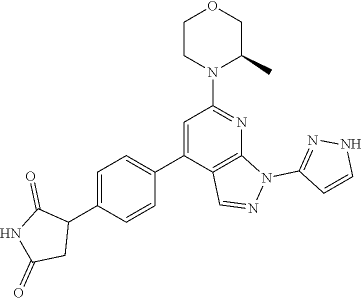 [SMILES: C[C@@H]1COCCN1c1cc(-c2ccc(C3CC(=O)NC3=O)cc2)c2cnn(-c3cc[nH]n3)c2n1] |
| 45 | 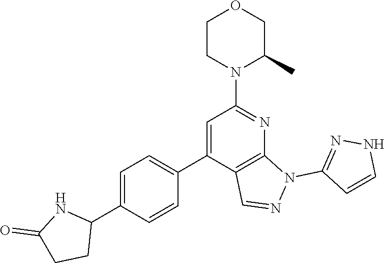 [SMILES: C[C@@H]1COCCN1c1cc(-c2ccc(C3CCC(=O)N3)cc2)c2cnn(-c3cc[nH]n3)c2n1] | 46 | 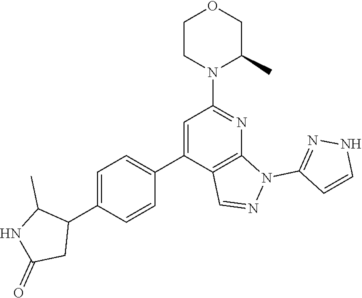 [SMILES: CC1NC(=O)CC1c1ccc(-c2cc(N3CCOC[C@H]3C)nc3c2cnn3-c2cc[nH]n2)cc1] |
| 47 | 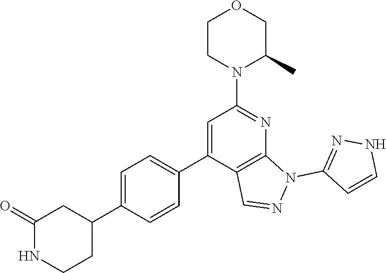 [SMILES: C[C@@H]1COCCN1c1cc(-c2ccc(C3CCNC(=O)C3)cc2)c2cnn(-c3cc[nH]n3)c2n1] | 48 | 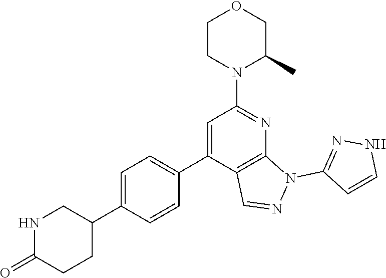 [SMILES: C[C@@H]1COCCN1c1cc(-c2ccc(C3CCC(=O)NC3)cc2)c2cnn(-c3cc[nH]n3)c2n1] |
| 49 | 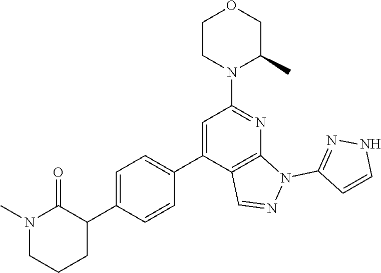 [SMILES: C[C@@H]1COCCN1c1cc(-c2ccc(C3CCCN(C)C3=O)cc2)c2cnn(-c3cc[nH]n3)c2n1] | 50 | 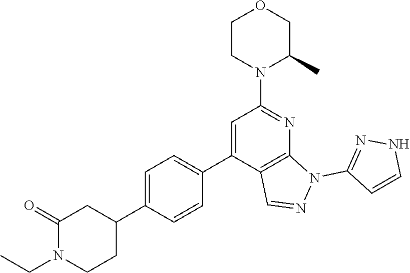 [SMILES: CCN1CCC(c2ccc(-c3cc(N4CCOC[C@H]4C)nc4c3cnn4-c3cc[nH]n3)cc2)CC1=O] |
| 51 | 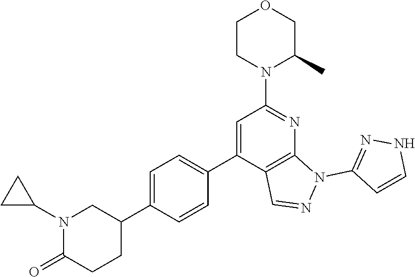 [SMILES: C[C@@H]1COCCN1c1cc(-c2ccc(C3CCC(=O)N(C4CC4)C3)cc2)c2cnn(-c3cc[nH]n3)c2n1] | 52 | 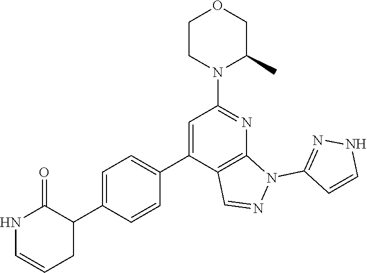 [SMILES: C[C@@H]1COCCN1c1cc(-c2ccc(C3CC=CNC3=O)cc2)c2cnn(-c3cc[nH]n3)c2n1] |
| 53 | 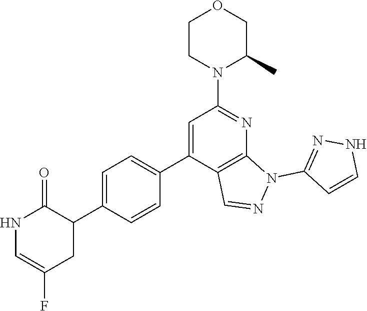 [SMILES: C[C@@H]1COCCN1c1cc(-c2ccc(C3CC(F)=CNC3=O)cc2)c2cnn(-c3cc[nH]n3)c2n1] | 54 | 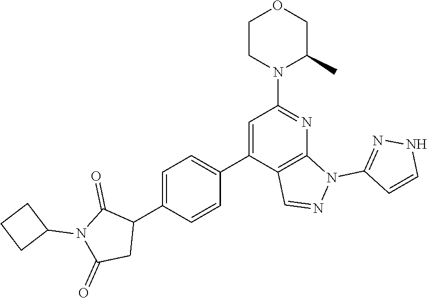 [SMILES: C[C@@H]1COCCN1c1cc(-c2ccc(C3CC(=O)N(C4CCC4)C3=O)cc2)c2cnn(-c3cc[nH]n3)c2n1] |
| 57 | 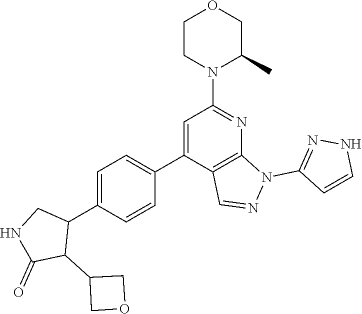 [SMILES: C[C@@H]1COCCN1c1cc(-c2ccc(C3CNC(=O)C3C3COC3)cc2)c2cnn(-c3cc[nH]n3)c2n1] | 58 | 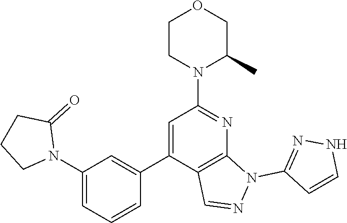 [SMILES: C[C@@H]1COCCN1c1cc(-c2cccc(N3CCCC3=O)c2)c2cnn(-c3cc[nH]n3)c2n1] |
| 59 | 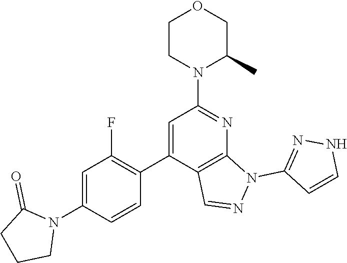 [SMILES: C[C@@H]1COCCN1c1cc(-c2ccc(N3CCCC3=O)cc2F)c2cnn(-c3cc[nH]n3)c2n1] | 60 | 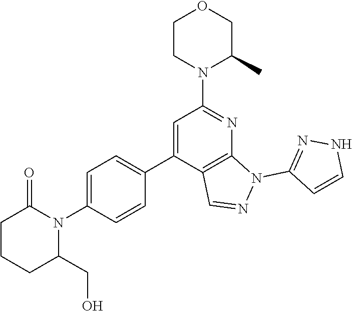 [SMILES: C[C@@H]1COCCN1c1cc(-c2ccc(N3C(=O)CCCC3CO)cc2)c2cnn(-c3cc[nH]n3)c2n1] |
| 61 | 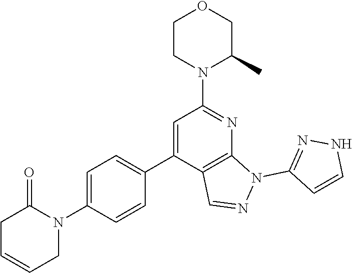 [SMILES: C[C@@H]1COCCN1c1cc(-c2ccc(N3CC=CCC3=O)cc2)c2cnn(-c3cc[nH]n3)c2n1] | 62 | 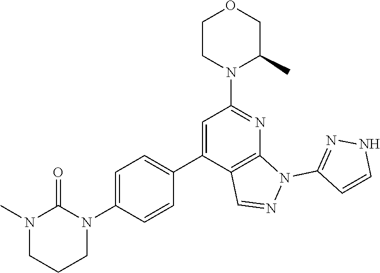 [SMILES: C[C@@H]1COCCN1c1cc(-c2ccc(N3CCCN(C)C3=O)cc2)c2cnn(-c3cc[nH]n3)c2n1] |
| 63 | 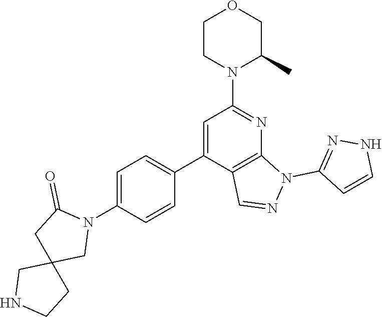 [SMILES: C[C@@H]1COCCN1c1cc(-c2ccc(N3CC4(CCNC4)CC3=O)cc2)c2cnn(-c3cc[nH]n3)c2n1] | 64 | 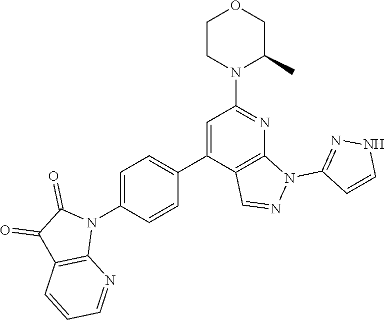 [SMILES: C[C@@H]1COCCN1c1cc(-c2ccc(N3C(=O)C(=O)c4cccnc43)cc2)c2cnn(-c3cc[nH]n3)c2n1] |
| 65 | 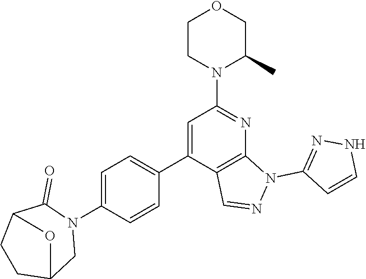 [SMILES: C[C@@H]1COCCN1c1cc(-c2ccc(N3CC4CCC(O4)C3=O)cc2)c2cnn(-c3cc[nH]n3)c2n1] | 66 | 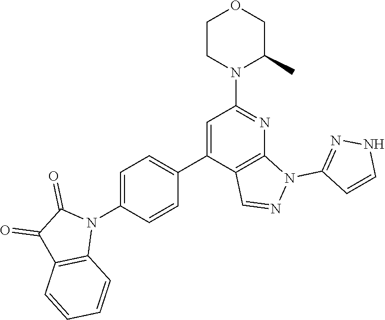 [SMILES: C[C@@H]1COCCN1c1cc(-c2ccc(N3C(=O)C(=O)c4ccccc43)cc2)c2cnn(-c3cc[nH]n3)c2n1] |
| 67 | 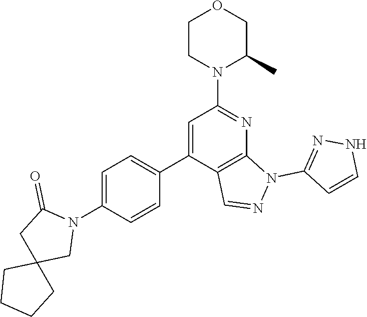 [SMILES: C[C@@H]1COCCN1c1cc(-c2ccc(N3CC4(CCCC4)CC3=O)cc2)c2cnn(-c3cc[nH]n3)c2n1] | 68 | 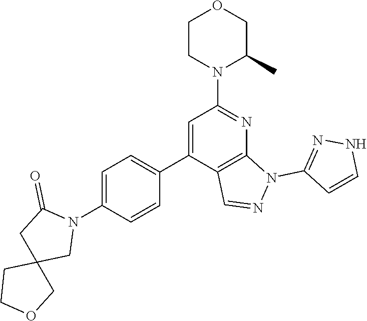 [SMILES: C[C@@H]1COCCN1c1cc(-c2ccc(N3CC4(CCOC4)CC3=O)cc2)c2cnn(-c3cc[nH]n3)c2n1] |
| 69 | 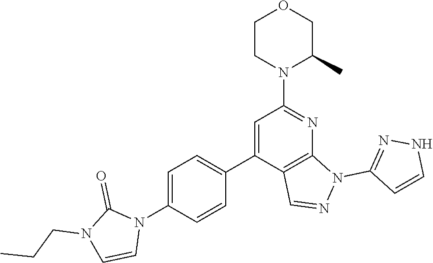 | 70 | 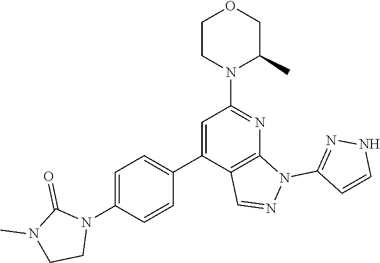 |
| 71 | 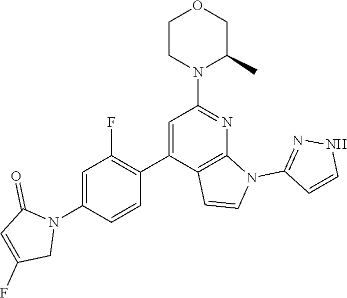 | 72 | 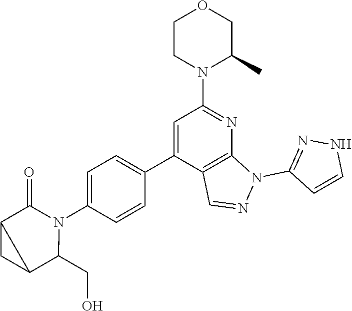 |
| 73 | 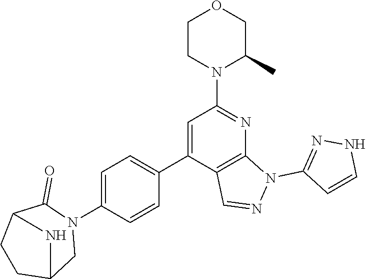 | 74 | 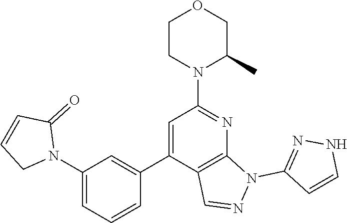 |
| 75 | 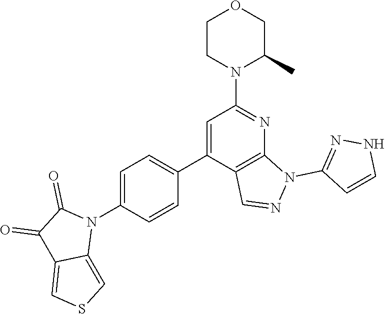 | 76 | 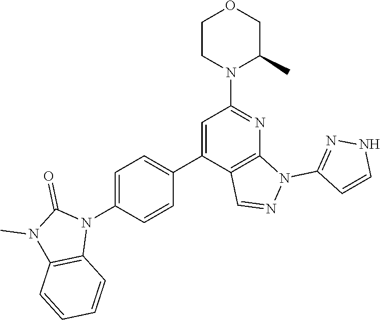 |
| 77 | 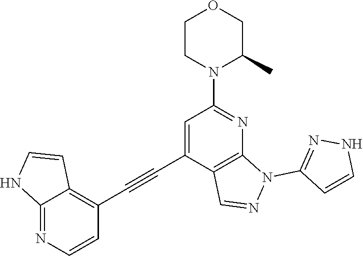 | 78 | 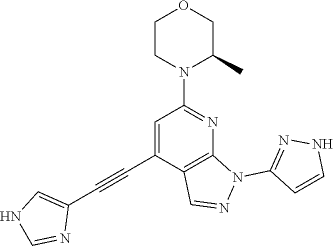 |
| 79 | 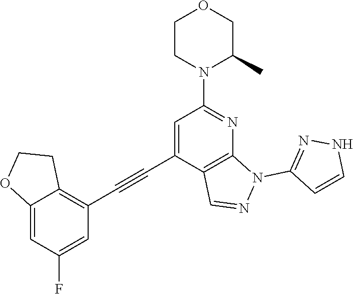 | 80 | 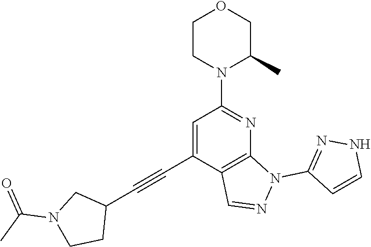 |
| 81 | 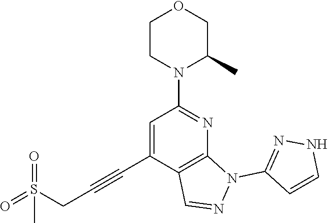 | 82 | 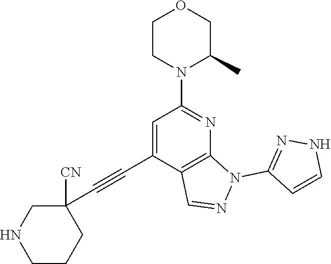 |
| 83 | 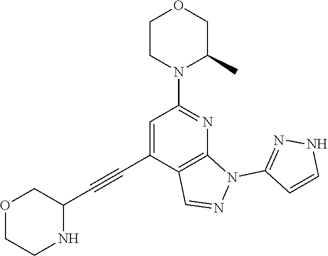 | 84 | 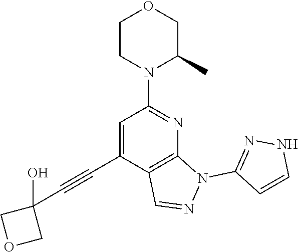 |
| 85 | 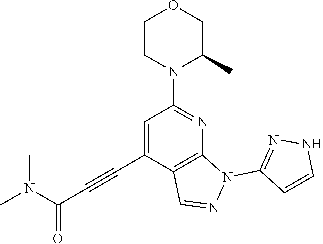 | 86 | 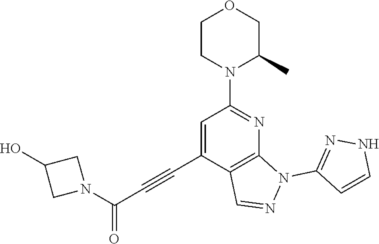 |
| 87 | 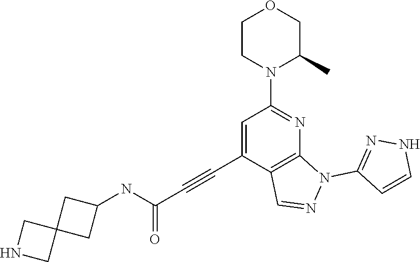 | 88 | 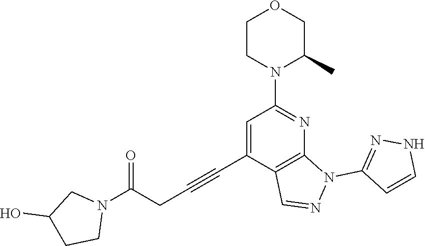 |
| 89 | 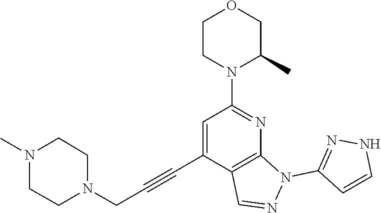 | 90 | 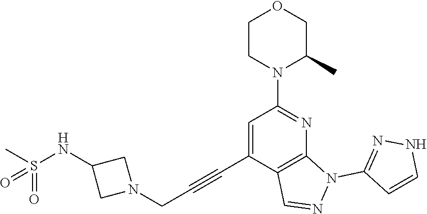 |
| 91 | 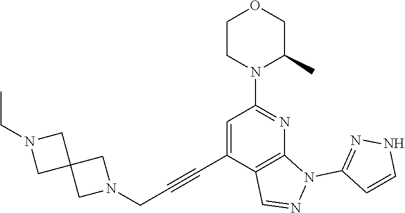 | 92 | 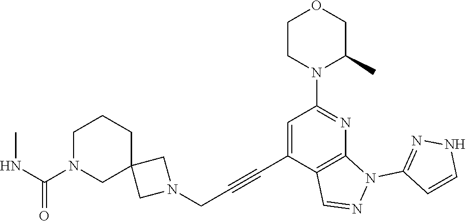 |
| 93 | 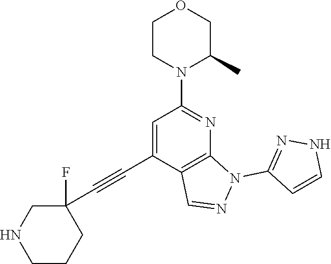 | 94 | 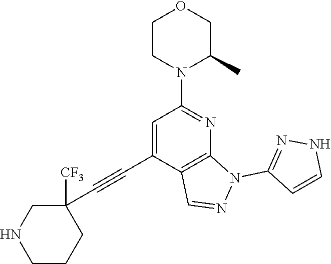 |
| 95 | 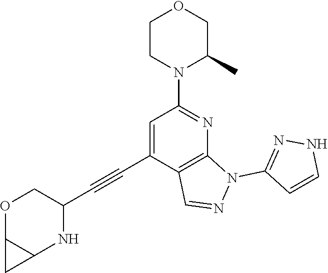 | 96 | 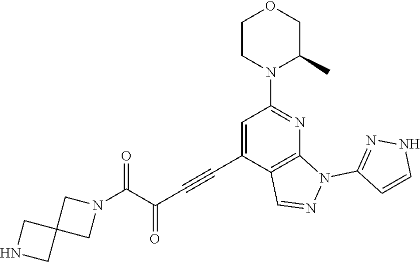 |
| 97 | 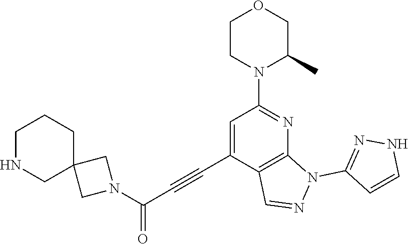 | 98 | 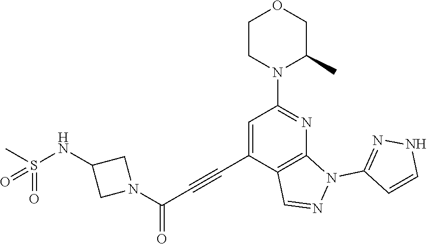 |
| 99 | 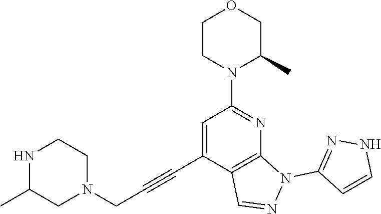 | 100 | 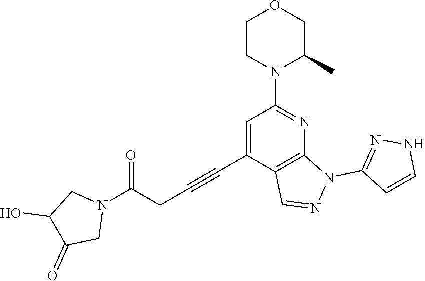 |
| 101 | 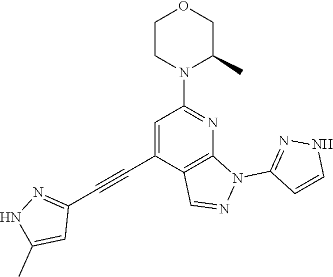 | 102 | 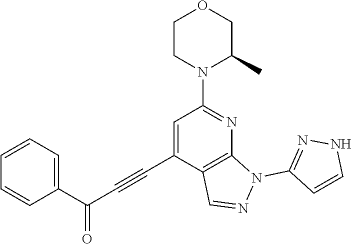 |
| 103 | 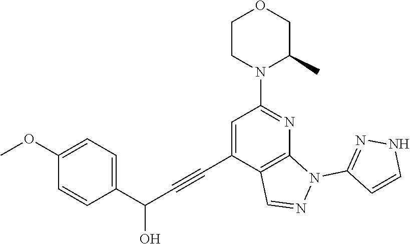 | 104 | 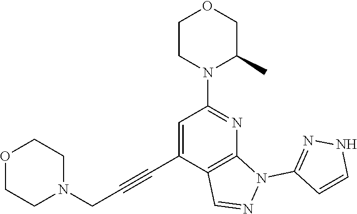 |
| 105 | 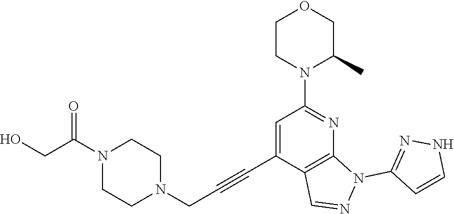 | 106 | 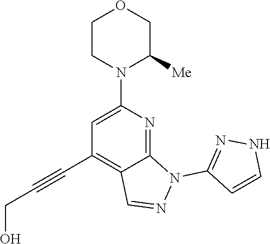 |
| 107 | 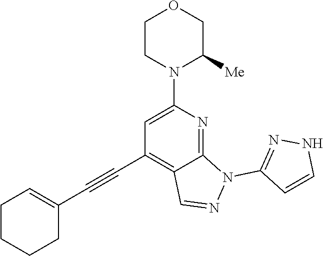 | 108 | 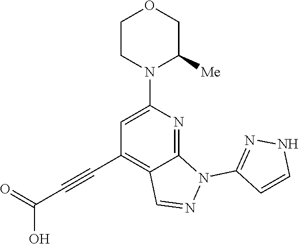 |
| 109 | 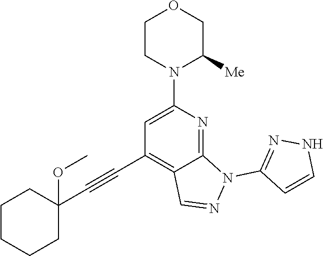 | 110 | 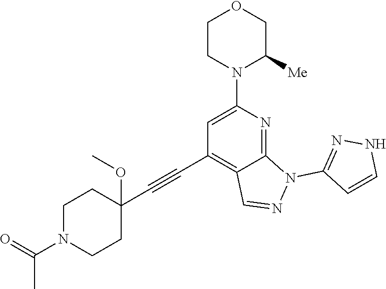 |
| 111 | 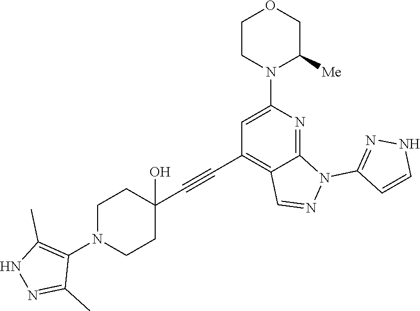 | 112 | 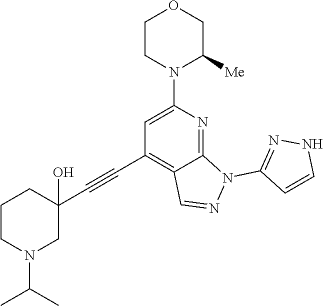 |
| 113 | 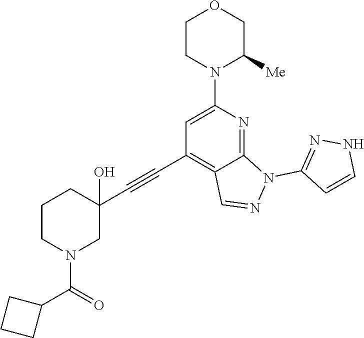 | 114 | 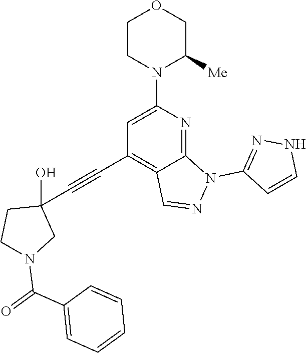 |
| 115 | 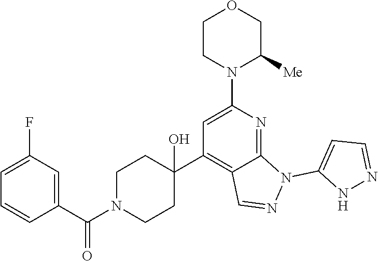 [SMILES: C[C@@H]1COCCN1c1cc(C2(O)CCN(C(=O)c3cccc(F)c3)CC2)c2cnn(-c3ccn[nH]3)c2n1] | 116 | 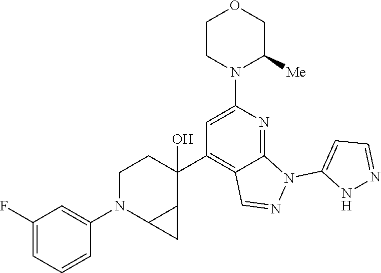 [SMILES: C[C@@H]1COCCN1c1cc(C2(O)CCN(c3cccc(F)c3)C3CC32)c2cnn(-c3ccn[nH]3)c2n1] |
| 117 | 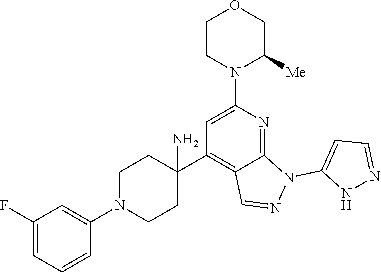 [SMILES: C[C@@H]1COCCN1c1cc(C2(N)CCN(c3cccc(F)c3)CC2)c2cnn(-c3ccn[nH]3)c2n1] | 118 | 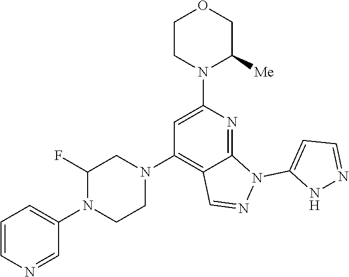 [SMILES: C[C@@H]1COCCN1c1cc(N2CCN(c3cccnc3)C(F)C2)c2cnn(-c3ccn[nH]3)c2n1] |
| 119 | 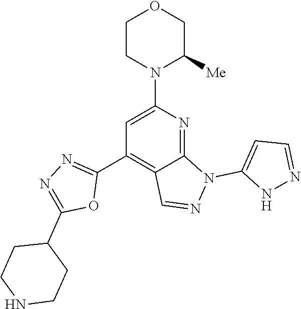 [SMILES: C[C@@H]1COCCN1c1cc(-c2nnc(C3CCNCC3)o2)c2cnn(-c3ccn[nH]3)c2n1] | 120 | 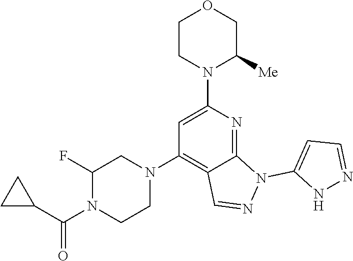 [SMILES: C[C@@H]1COCCN1c1cc(N2CCN(C(=O)C3CC3)C(F)C2)c2cnn(-c3ccn[nH]3)c2n1] |
| 121 | 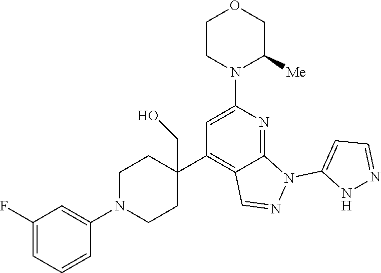 [SMILES: C[C@@H]1COCCN1c1cc(C2(CO)CCN(c3cccc(F)c3)CC2)c2cnn(-c3ccn[nH]3)c2n1] | 122 | 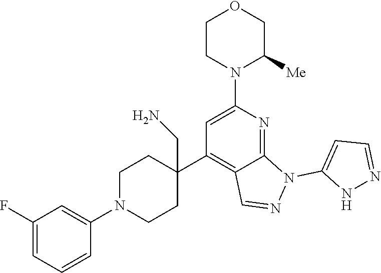 [SMILES: C[C@@H]1COCCN1c1cc(C2(CN)CCN(c3cccc(F)c3)CC2)c2cnn(-c3ccn[nH]3)c2n1] |
| 123 | 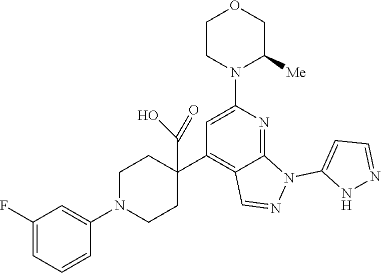 [SMILES: C[C@@H]1COCCN1c1cc(C2(C(=O)O)CCN(c3cccc(F)c3)CC2)c2cnn(-c3ccn[nH]3)c2n1] | 124 | 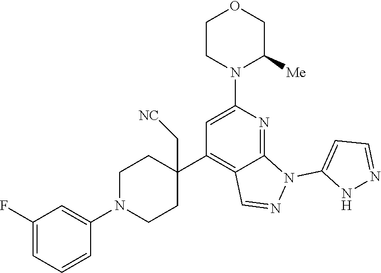 [SMILES: C[C@@H]1COCCN1c1cc(C2(CC#N)CCN(c3cccc(F)c3)CC2)c2cnn(-c3ccn[nH]3)c2n1] |
| 125 | 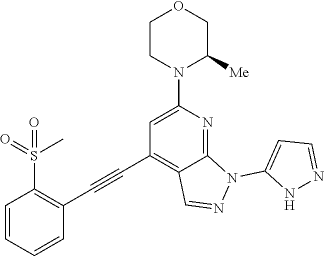 [SMILES: C[C@@H]1COCCN1c1cc(C#Cc2ccccc2S(C)(=O)=O)c2cnn(-c3ccn[nH]3)c2n1] | 126 | 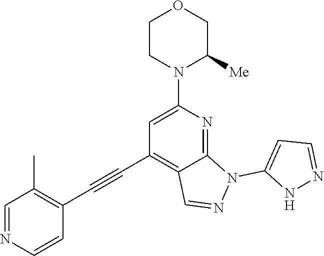 [SMILES: Cc1cnccc1C#Cc1cc(N2CCOC[C@H]2C)nc2c1cnn2-c1ccn[nH]1] |
| 127 | 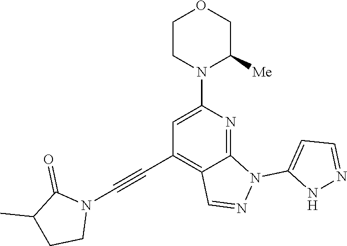 [SMILES: CC1CCN(C#Cc2cc(N3CCOC[C@H]3C)nc3c2cnn3-c2ccn[nH]2)C1=O] | 128 | 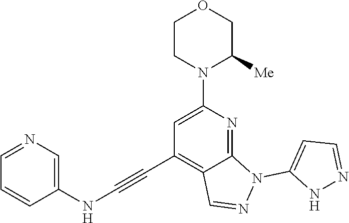 [SMILES: C[C@@H]1COCCN1c1cc(C#CNc2cccnc2)c2cnn(-c3ccn[nH]3)c2n1] |
| 129 | 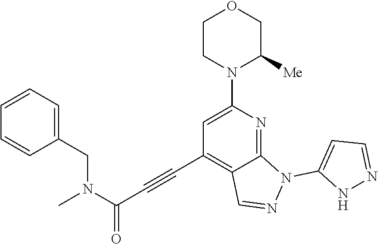 [SMILES: C[C@@H]1COCCN1c1cc(C#CC(=O)N(C)Cc2ccccc2)c2cnn(-c3ccn[nH]3)c2n1] | 130 | 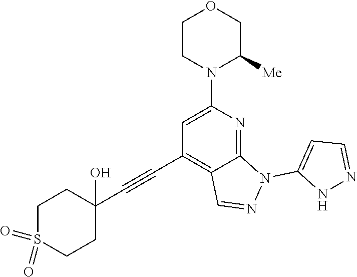 [SMILES: C[C@@H]1COCCN1c1cc(C#CC2(O)CCS(=O)(=O)CC2)c2cnn(-c3ccn[nH]3)c2n1] |
| 131 | 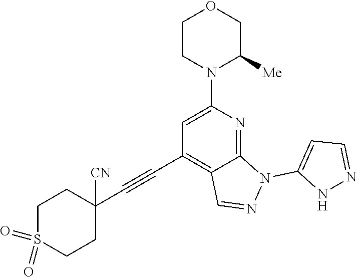 [SMILES: C[C@@H]1COCCN1c1cc(C#CC2(C#N)CCS(=O)(=O)CC2)c2cnn(-c3ccn[nH]3)c2n1] | 132 | 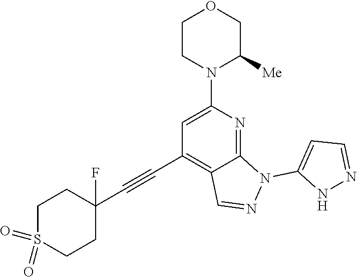 [SMILES: C[C@@H]1COCCN1c1cc(C#CC2(F)CCS(=O)(=O)CC2)c2cnn(-c3ccn[nH]3)c2n1] |
| 133 | 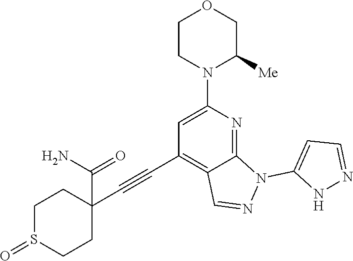 [SMILES: C[C@@H]1COCCN1c1cc(C#CC2(C(N)=O)CCS(=O)CC2)c2cnn(-c3ccn[nH]3)c2n1] | 134 | 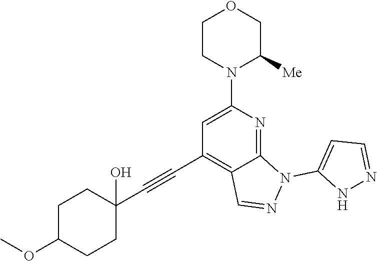 [SMILES: COC1CCC(O)(C#Cc2cc(N3CCOC[C@H]3C)nc3c2cnn3-c2ccn[nH]2)CC1] |
| 135 | 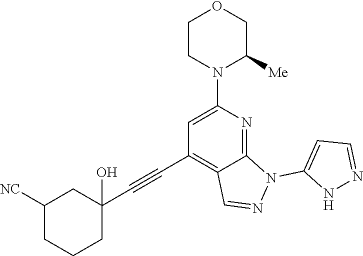 [SMILES: C[C@@H]1COCCN1c1cc(C#CC2(O)CCCC(C#N)C2)c2cnn(-c3ccn[nH]3)c2n1] | 136 | 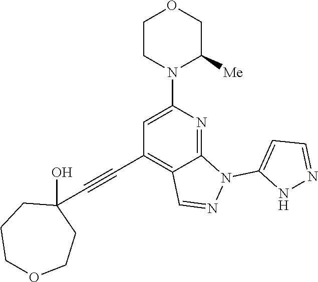 [SMILES: C[C@@H]1COCCN1c1cc(C#CC2(O)CCCOCC2)c2cnn(-c3ccn[nH]3)c2n1] |
| 137 | 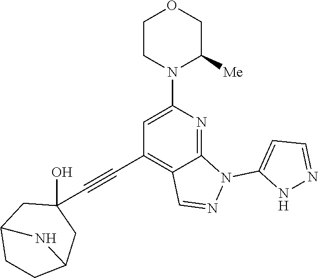 [SMILES: C[C@@H]1COCCN1c1cc(C#CC2(O)CC3CCC(C2)N3)c2cnn(-c3ccn[nH]3)c2n1] | 138 | 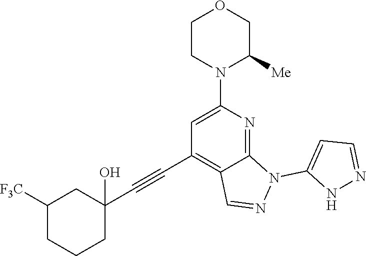 [SMILES: C[C@@H]1COCCN1c1cc(C#CC2(O)CCCC(C(F)(F)F)C2)c2cnn(-c3ccn[nH]3)c2n1] |
| 139 | 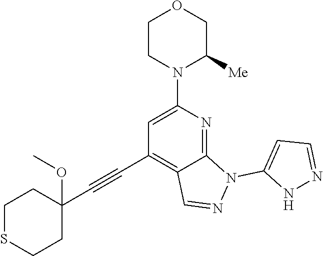 [SMILES: COC1(C#Cc2cc(N3CCOC[C@H]3C)nc3c2cnn3-c2ccn[nH]2)CCSCC1] | 140 | 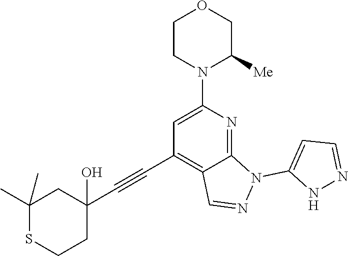 [SMILES: C[C@@H]1COCCN1c1cc(C#CC2(O)CCSC(C)(C)C2)c2cnn(-c3ccn[nH]3)c2n1] |
| 141 | 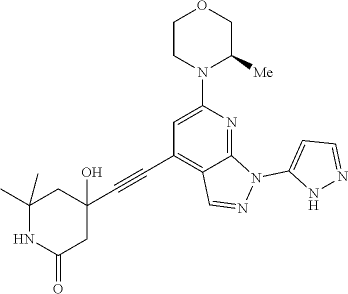 [SMILES: C[C@@H]1COCCN1c1cc(C#CC2(O)CC(=O)NC(C)(C)C2)c2cnn(-c3ccn[nH]3)c2n1] | 142 | 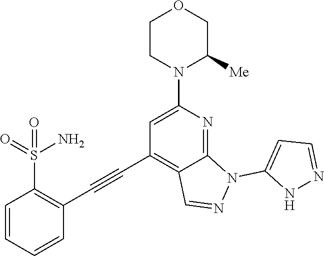 [SMILES: C[C@@H]1COCCN1c1cc(C#Cc2ccccc2S(N)(=O)=O)c2cnn(-c3ccn[nH]3)c2n1] |
| 143 | 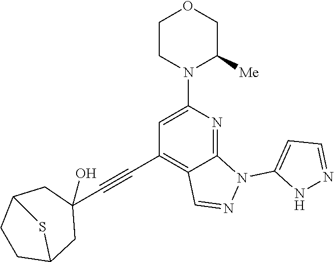 [SMILES: C[C@@H]1COCCN1c1cc(C#CC2(O)CC3CCC(C2)S3)c2cnn(-c3ccn[nH]3)c2n1] | 144 | 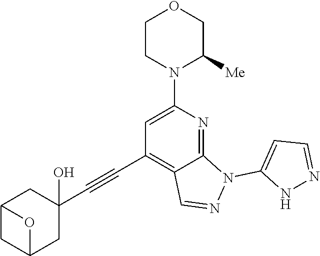 [SMILES: C[C@@H]1COCCN1c1cc(C#CC2(O)CC3CC(C2)O3)c2cnn(-c3ccn[nH]3)c2n1] |
| 145 | 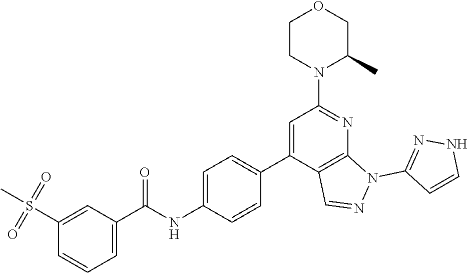 [SMILES: C[C@@H]1COCCN1c1cc(-c2ccc(NC(=O)c3cccc(S(C)(=O)=O)c3)cc2)c2cnn(-c3cc[nH]n3)c2n1] | 146 | 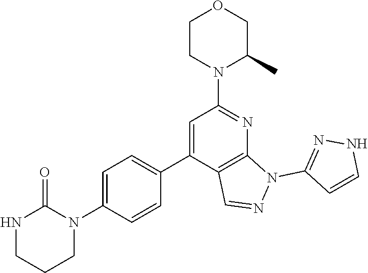 [SMILES: C[C@@H]1COCCN1c1cc(-c2ccc(N3CCCNC3=O)cc2)c2cnn(-c3cc[nH]n3)c2n1] |
| 147 | 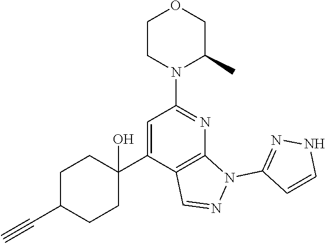 [SMILES: C#CC1CCC(O)(c2cc(N3CCOC[C@H]3C)nc3c2cnn3-c2cc[nH]n2)CC1] | 148 | 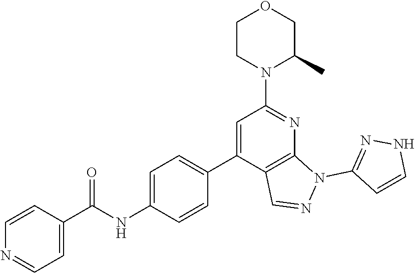 [SMILES: C[C@@H]1COCCN1c1cc(-c2ccc(NC(=O)c3ccncc3)cc2)c2cnn(-c3cc[nH]n3)c2n1] |
| 149 | 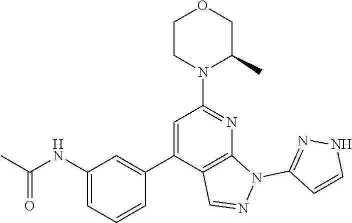 [SMILES: CC(=O)Nc1cccc(-c2cc(N3CCOC[C@H]3C)nc3c2cnn3-c2cc[nH]n2)c1] | 150 | 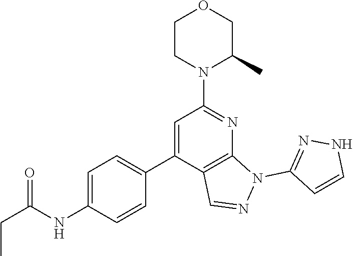 [SMILES: CCC(=O)Nc1ccc(-c2cc(N3CCOC[C@H]3C)nc3c2cnn3-c2cc[nH]n2)cc1] |
| 151 | 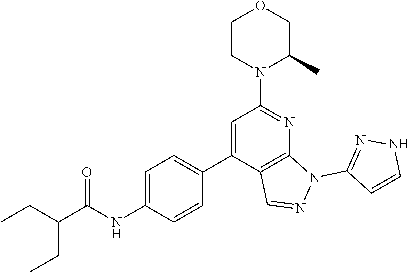 [SMILES: CCC(CC)C(=O)Nc1ccc(-c2cc(N3CCOC[C@H]3C)nc3c2cnn3-c2cc[nH]n2)cc1] | 152 | 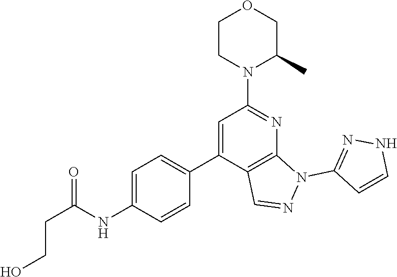 [SMILES: C[C@@H]1COCCN1c1cc(-c2ccc(NC(=O)CCO)cc2)c2cnn(-c3cc[nH]n3)c2n1] |
| 153 | 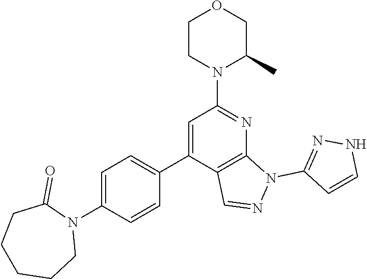 [SMILES: C[C@@H]1COCCN1c1cc(-c2ccc(N3CCCCCC3=O)cc2)c2cnn(-c3cc[nH]n3)c2n1] | 154 | 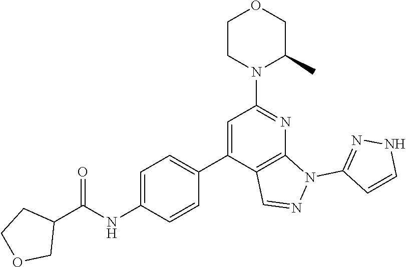 [SMILES: C[C@@H]1COCCN1c1cc(-c2ccc(NC(=O)C3CCOC3)cc2)c2cnn(-c3cc[nH]n3)c2n1] |
| 155 | 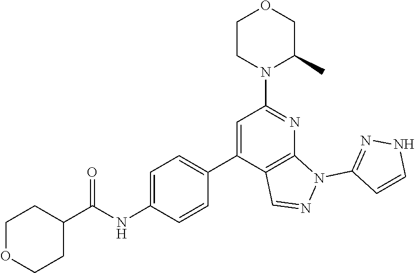 [SMILES: C[C@@H]1COCCN1c1cc(-c2ccc(NC(=O)C3CCOCC3)cc2)c2cnn(-c3cc[nH]n3)c2n1] | 156 | 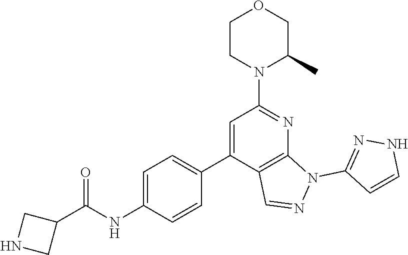 [SMILES: C[C@@H]1COCCN1c1cc(-c2ccc(NC(=O)C3CNC3)cc2)c2cnn(-c3cc[nH]n3)c2n1] |
| 157 | 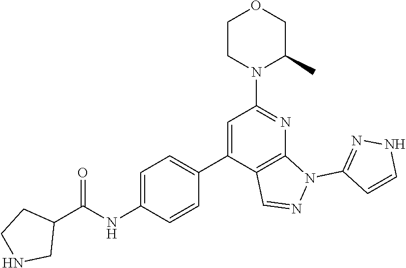 [SMILES: C[C@@H]1COCCN1c1cc(-c2ccc(NC(=O)C3CCNC3)cc2)c2cnn(-c3cc[nH]n3)c2n1] | 158 | 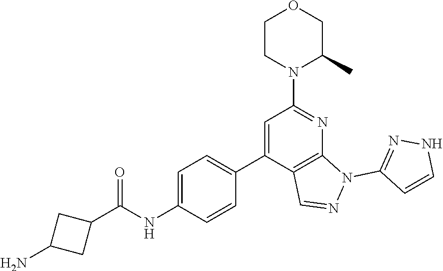 [SMILES: C[C@@H]1COCCN1c1cc(-c2ccc(NC(=O)C3CC(N)C3)cc2)c2cnn(-c3cc[nH]n3)c2n1] |
| 159 | 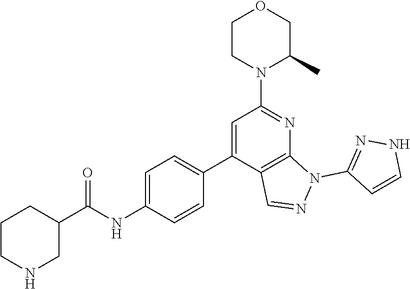 [SMILES: C[C@@H]1COCCN1c1cc(-c2ccc(NC(=O)C3CCCNC3)cc2)c2cnn(-c3cc[nH]n3)c2n1] | 160 | 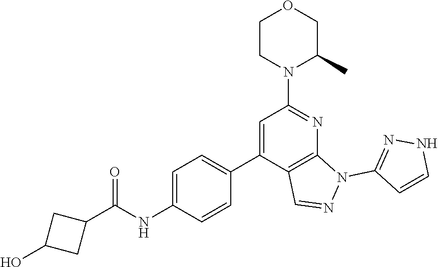 [SMILES: C[C@@H]1COCCN1c1cc(-c2ccc(NC(=O)C3CC(O)C3)cc2)c2cnn(-c3cc[nH]n3)c2n1] |
| 161 | 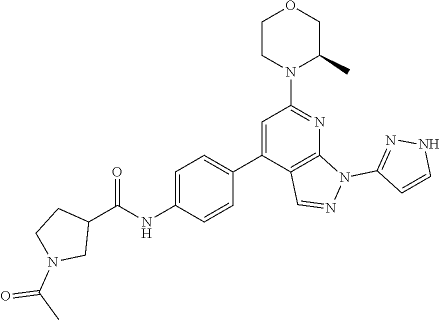 [SMILES: CC(=O)N1CCC(C(=O)Nc2ccc(-c3cc(N4CCOC[C@H]4C)nc4c3cnn4-c3cc[nH]n3)cc2)C1] | 162 | 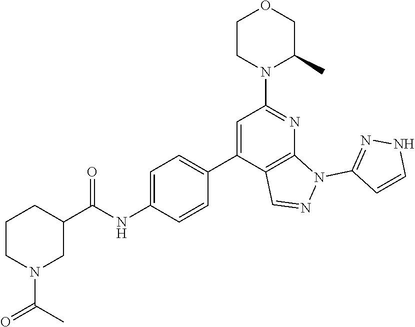 [SMILES: CC(=O)N1CCCC(C(=O)Nc2ccc(-c3cc(N4CCOC[C@H]4C)nc4c3cnn4-c3cc[nH]n3)cc2)C1] |
| 163 | 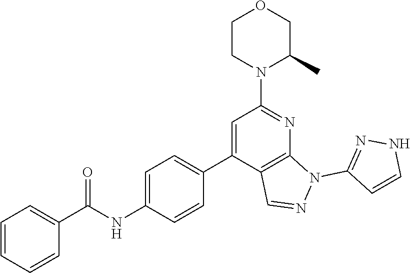 [SMILES: C[C@@H]1COCCN1c1cc(-c2ccc(NC(=O)c3ccccc3)cc2)c2cnn(-c3cc[nH]n3)c2n1] | 164 | 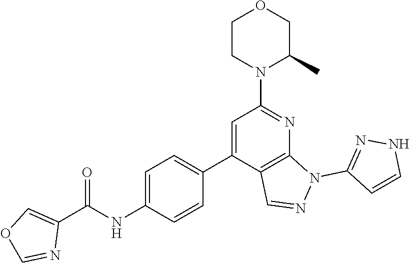 [SMILES: C[C@@H]1COCCN1c1cc(-c2ccc(NC(=O)c3cocn3)cc2)c2cnn(-c3cc[nH]n3)c2n1] |
| 165 | 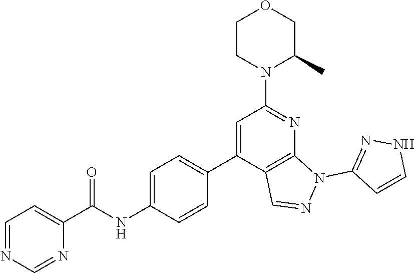 [SMILES: C[C@@H]1COCCN1c1cc(-c2ccc(NC(=O)c3ccncn3)cc2)c2cnn(-c3cc[nH]n3)c2n1] | 166 | 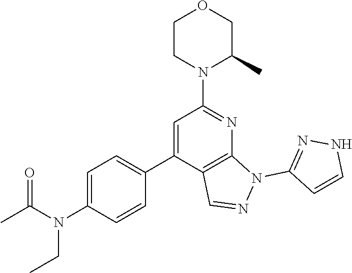 [SMILES: CCN(C(C)=O)c1ccc(-c2cc(N3CCOC[C@H]3C)nc3c2cnn3-c2cc[nH]n2)cc1] |
| 167 | 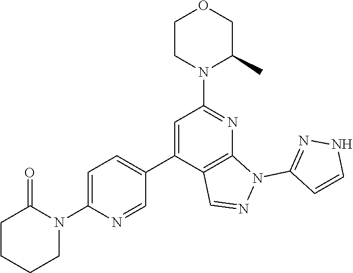 [SMILES: C[C@@H]1COCCN1c1cc(-c2ccc(N3CCCCC3=O)nc2)c2cnn(-c3cc[nH]n3)c2n1] | 168 | 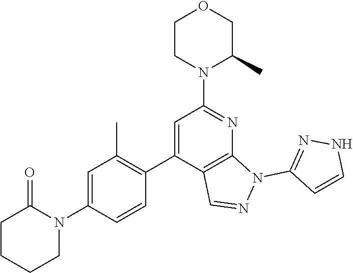 [SMILES: Cc1cc(N2CCCCC2=O)ccc1-c1cc(N2CCOC[C@H]2C)nc2c1cnn2-c1cc[nH]n1] |
| 169 | 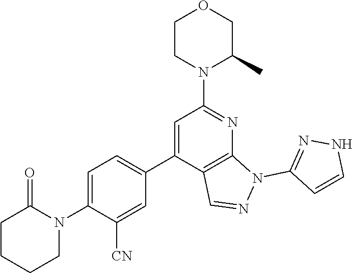 [SMILES: C[C@@H]1COCCN1c1cc(-c2ccc(N3CCCCC3=O)c(C#N)c2)c2cnn(-c3cc[nH]n3)c2n1] | 170 | 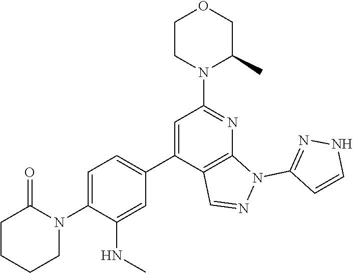 [SMILES: CNc1cc(-c2cc(N3CCOC[C@H]3C)nc3c2cnn3-c2cc[nH]n2)ccc1N1CCCCC1=O] |
| 171 | 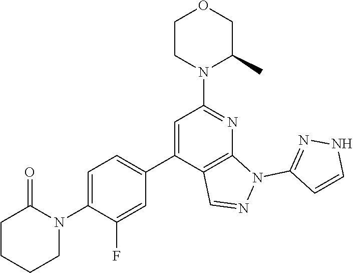 [SMILES: C[C@@H]1COCCN1c1cc(-c2ccc(N3CCCCC3=O)c(F)c2)c2cnn(-c3cc[nH]n3)c2n1] | 172 | 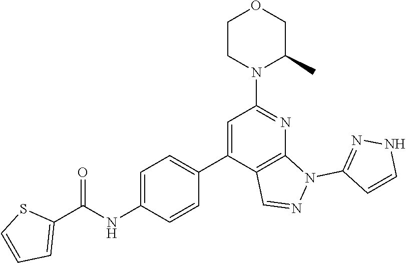 [SMILES: C[C@@H]1COCCN1c1cc(-c2ccc(NC(=O)c3cccs3)cc2)c2cnn(-c3cc[nH]n3)c2n1] |
| 173 | 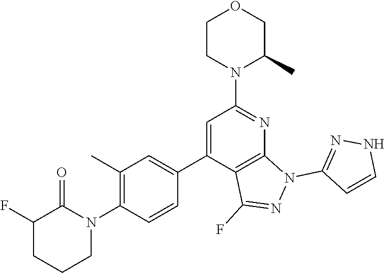 [SMILES: Cc1cc(-c2cc(N3CCOC[C@H]3C)nc3c2c(F)nn3-c2cc[nH]n2)ccc1N1CCCC(F)C1=O] | 174 | 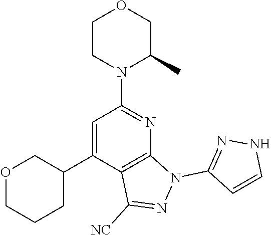 [SMILES: C[C@@H]1COCCN1c1cc(C2CCCOC2)c2c(C#N)nn(-c3cc[nH]n3)c2n1] |
| 175 | 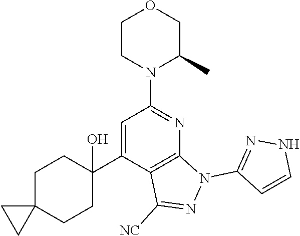 [SMILES: C[C@@H]1COCCN1c1cc(C2(O)CCC3(CC3)CC2)c2c(C#N)nn(-c3cc[nH]n3)c2n1] | 176 | 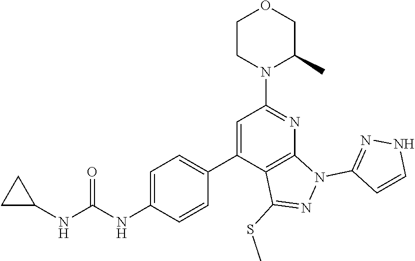 [SMILES: CSc1nn(-c2cc[nH]n2)c2nc(N3CCOC[C@H]3C)cc(-c3ccc(NC(=O)NC4CC4)cc3)c12] |
| 177 | 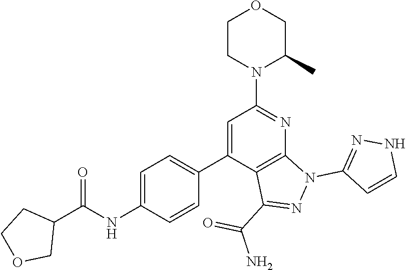 [SMILES: C[C@@H]1COCCN1c1cc(-c2ccc(NC(=O)C3CCOC3)cc2)c2c(C(N)=O)nn(-c3cc[nH]n3)c2n1] | 178 | 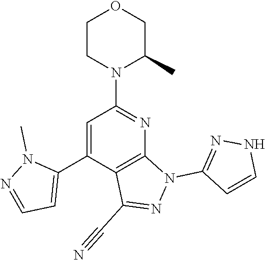 [SMILES: C[C@@H]1COCCN1c1cc(-c2ccnn2C)c2c(C#N)nn(-c3cc[nH]n3)c2n1] |
| 179 | 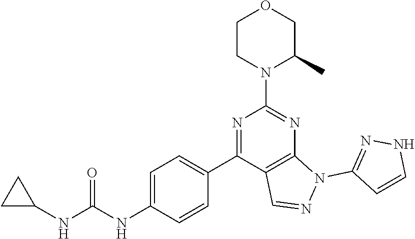 [SMILES: C[C@@H]1COCCN1c1nc(-c2ccc(NC(=O)NC3CC3)cc2)c2cnn(-c3cc[nH]n3)c2n1] | 180 | 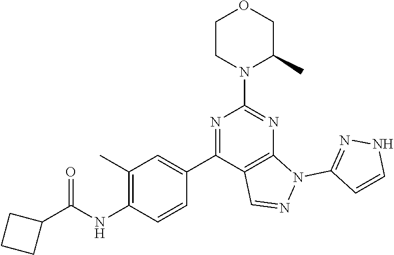 [SMILES: Cc1cc(-c2nc(N3CCOC[C@H]3C)nc3c2cnn3-c2cc[nH]n2)ccc1NC(=O)C1CCC1] |
| 181 | 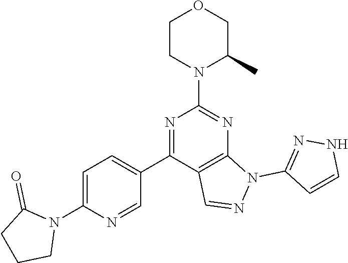 [SMILES: C[C@@H]1COCCN1c1nc(-c2ccc(N3CCCC3=O)nc2)c2cnn(-c3cc[nH]n3)c2n1] | 182 | 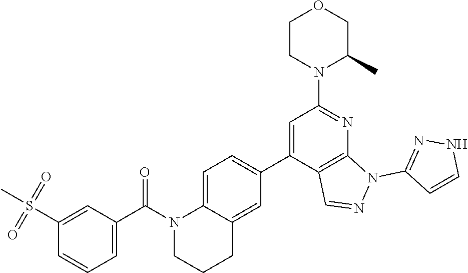 [SMILES: C[C@@H]1COCCN1c1cc(-c2ccc3c(c2)CCCN3C(=O)c2cccc(S(C)(=O)=O)c2)c2cnn(-c3cc[nH]n3)c2n1] |
| 183 | 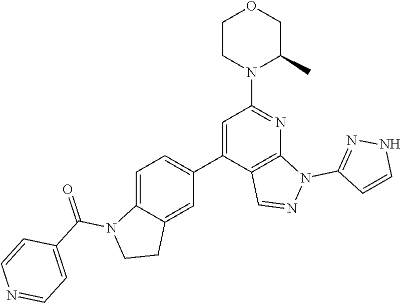 [SMILES: C[C@@H]1COCCN1c1cc(-c2ccc3c(c2)CCN3C(=O)c2ccncc2)c2cnn(-c3cc[nH]n3)c2n1] | 184 | 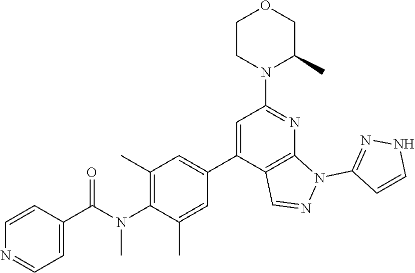 [SMILES: Cc1cc(-c2cc(N3CCOC[C@H]3C)nc3c2cnn3-c2cc[nH]n2)cc(C)c1N(C)C(=O)c1ccncc1] |
| 185 | 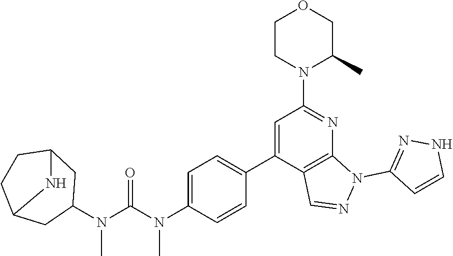 [SMILES: C[C@@H]1COCCN1c1cc(-c2ccc(N(C)C(=O)N(C)C3CC4CCC(C3)N4)cc2)c2cnn(-c3cc[nH]n3)c2n1] | 186 | 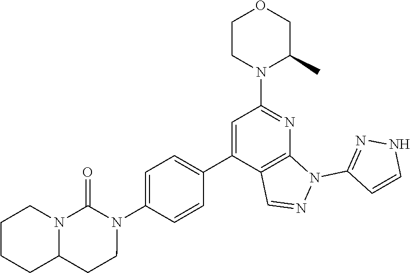 [SMILES: C[C@@H]1COCCN1c1cc(-c2ccc(N3CCC4CCCCN4C3=O)cc2)c2cnn(-c3cc[nH]n3)c2n1] |
| 187 | 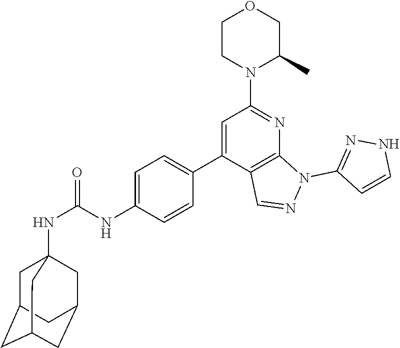 [SMILES: C[C@@H]1COCCN1c1cc(-c2ccc(NC(=O)NC34CC5CC(CC(C5)C3)C4)cc2)c2cnn(-c3cc[nH]n3)c2n1] | 188 | 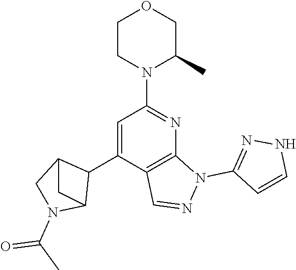 [SMILES: CC(=O)N1CC2CC1C2c1cc(N2CCOC[C@H]2C)nc2c1cnn2-c1cc[nH]n1] |
| 189 | 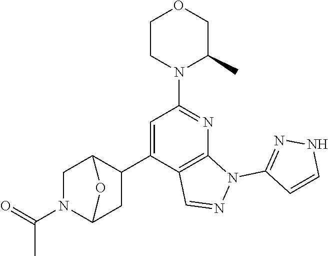 [SMILES: CC(=O)N1CC2OC1CC2c1cc(N2CCOC[C@H]2C)nc2c1cnn2-c1cc[nH]n1] | 190 | 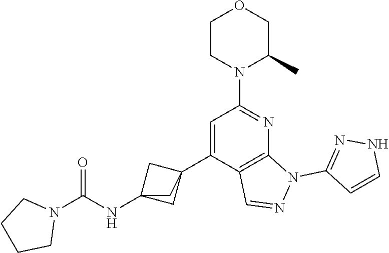 [SMILES: C[C@@H]1COCCN1c1cc(C23CC(NC(=O)N4CCCC4)(C2)C3)c2cnn(-c3cc[nH]n3)c2n1] |
| 191 | 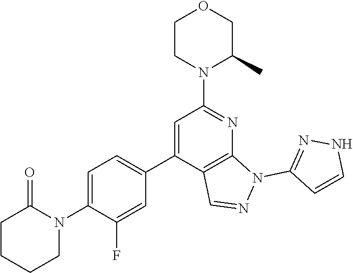 [SMILES: C[C@@H]1COCCN1c1cc(-c2ccc(N3CCCCC3=O)c(F)c2)c2cnn(-c3cc[nH]n3)c2n1] | 192 | 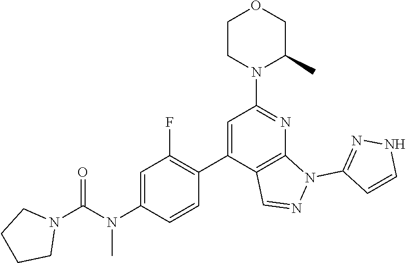 [SMILES: C[C@@H]1COCCN1c1cc(-c2ccc(N(C)C(=O)N3CCCC3)cc2F)c2cnn(-c3cc[nH]n3)c2n1] |
| 193 | 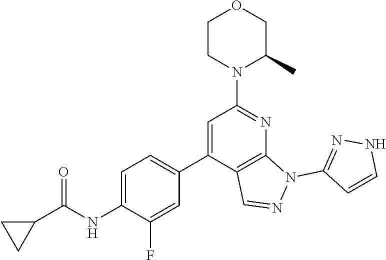 [SMILES: C[C@@H]1COCCN1c1cc(-c2ccc(NC(=O)C3CC3)c(F)c2)c2cnn(-c3cc[nH]n3)c2n1] | 194 | 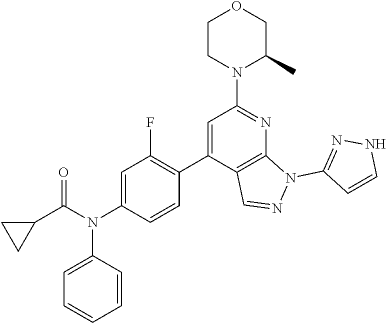 [SMILES: C[C@@H]1COCCN1c1cc(-c2ccc(N(C(=O)C3CC3)c3ccccc3)cc2F)c2cnn(-c3cc[nH]n3)c2n1] |
| 195 | 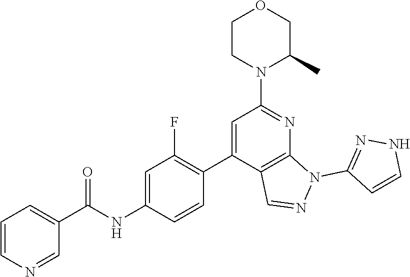 [SMILES: C[C@@H]1COCCN1c1cc(-c2ccc(NC(=O)c3cccnc3)cc2F)c2cnn(-c3cc[nH]n3)c2n1] | 196 | 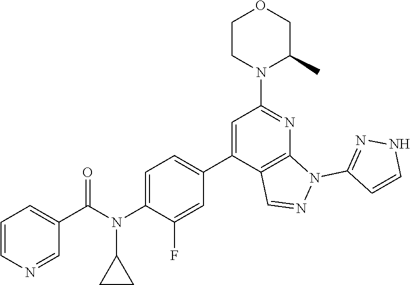 [SMILES: C[C@@H]1COCCN1c1cc(-c2ccc(N(C(=O)c3cccnc3)C3CC3)c(F)c2)c2cnn(-c3cc[nH]n3)c2n1] |
| 197 | 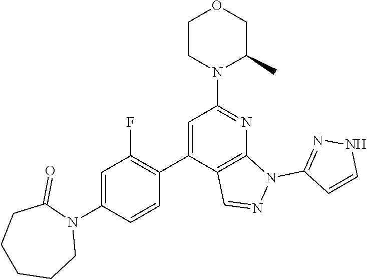 [SMILES: C[C@@H]1COCCN1c1cc(-c2ccc(N3CCCCCC3=O)cc2F)c2cnn(-c3cc[nH]n3)c2n1] | 198 | 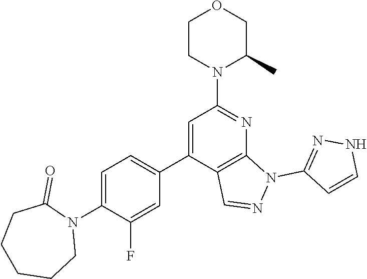 [SMILES: C[C@@H]1COCCN1c1cc(-c2ccc(N3CCCCCC3=O)c(F)c2)c2cnn(-c3cc[nH]n3)c2n1] |
| 199 | 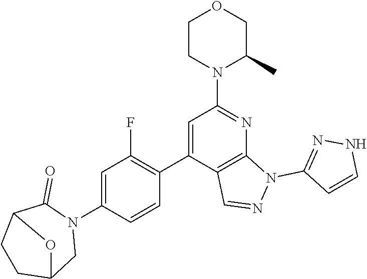 [SMILES: C[C@@H]1COCCN1c1cc(-c2ccc(N3CC4CCC(O4)C3=O)cc2F)c2cnn(-c3cc[nH]n3)c2n1] | 200 | 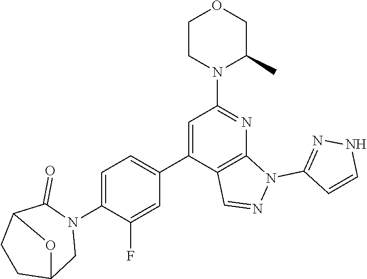 [SMILES: C[C@@H]1COCCN1c1cc(-c2ccc(N3CC4CCC(O4)C3=O)c(F)c2)c2cnn(-c3cc[nH]n3)c2n1] |
| 201 | 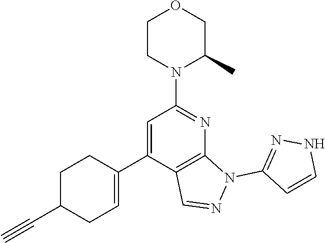 [SMILES: C#CC1CC=C(c2cc(N3CCOC[C@H]3C)nc3c2cnn3-c2cc[nH]n2)CC1] | 202 | 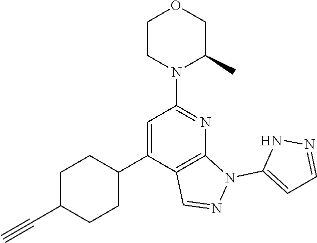 [SMILES: C#CC1CCC(c2cc(N3CCOC[C@H]3C)nc3c2cnn3-c2ccn[nH]2)CC1] |
| 203 | 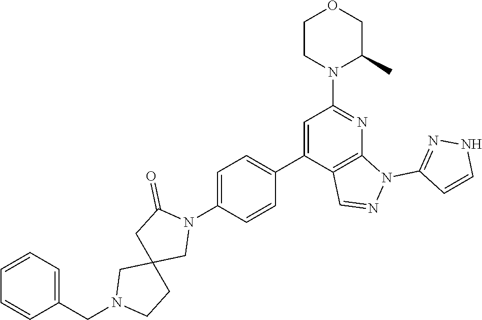 [SMILES: C[C@@H]1COCCN1c1cc(-c2ccc(N3CC4(CCN(Cc5ccccc5)C4)CC3=O)cc2)c2cnn(-c3cc[nH]n3)c2n1] | 204 | 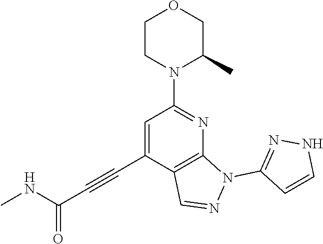 [SMILES: CNC(=O)C#Cc1cc(N2CCOC[C@H]2C)nc2c1cnn2-c1cc[nH]n1] |
| 205 | 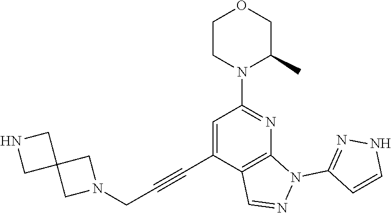 [SMILES: C[C@@H]1COCCN1c1cc(C#CCN2CC3(CNC3)C2)c2cnn(-c3cc[nH]n3)c2n1] | 206 | 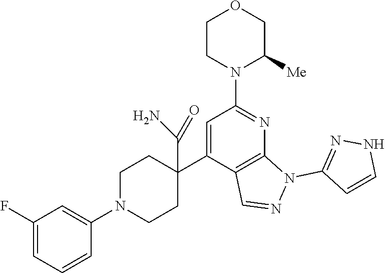 [SMILES: C[C@@H]1COCCN1c1cc(C2(C(N)=O)CCN(c3cccc(F)c3)CC2)c2cnn(-c3cc[nH]n3)c2n1] |
| 207 | 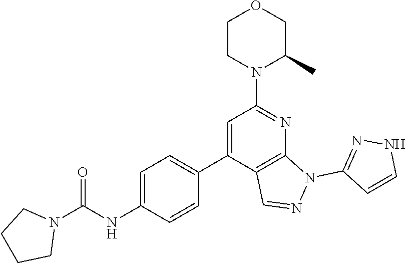 [SMILES: C[C@@H]1COCCN1c1cc(-c2ccc(NC(=O)N3CCCC3)cc2)c2cnn(-c3cc[nH]n3)c2n1] | 208 | 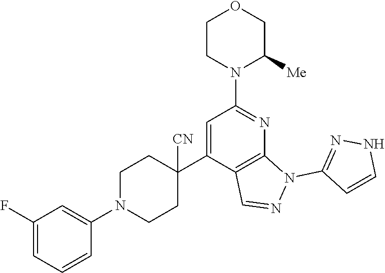 [SMILES: C[C@@H]1COCCN1c1cc(C2(C#N)CCN(c3cccc(F)c3)CC2)c2cnn(-c3cc[nH]n3)c2n1] |
| 209 | 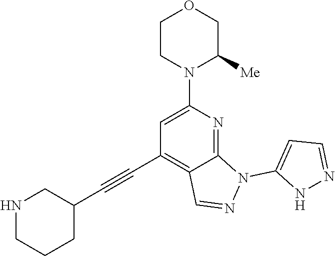 [SMILES: C[C@@H]1COCCN1c1cc(C#CC2CCCNC2)c2cnn(-c3ccn[nH]3)c2n1] | 210 | 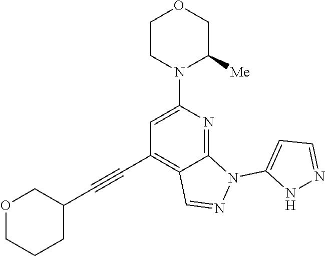 [SMILES: C[C@@H]1COCCN1c1cc(C#CC2CCCOC2)c2cnn(-c3ccn[nH]3)c2n1] |
| 211 | 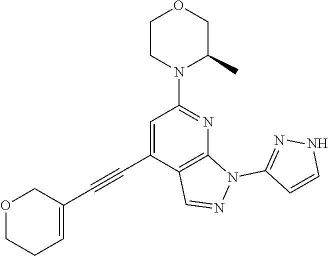 [SMILES: C[C@@H]1COCCN1c1cc(C#CC2=CCCOC2)c2cnn(-c3cc[nH]n3)c2n1] | 212 | 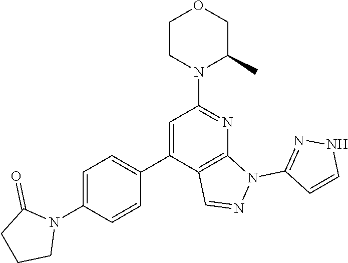 [SMILES: C[C@@H]1COCCN1c1cc(-c2ccc(N3CCCC3=O)cc2)c2cnn(-c3cc[nH]n3)c2n1] |
| 213 | 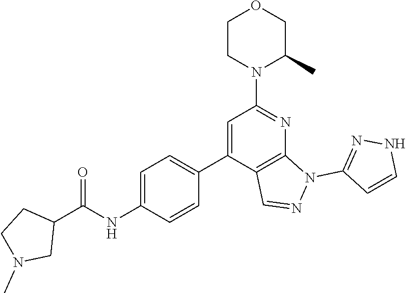 [SMILES: C[C@@H]1COCCN1c1cc(-c2ccc(NC(=O)C3CCN(C)C3)cc2)c2cnn(-c3cc[nH]n3)c2n1] | 214 | 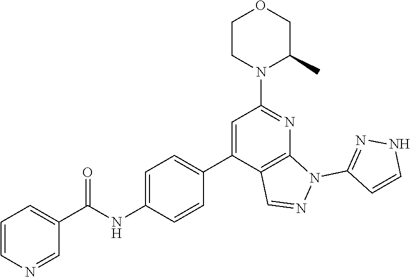 [SMILES: C[C@@H]1COCCN1c1cc(-c2ccc(NC(=O)c3cccnc3)cc2)c2cnn(-c3cc[nH]n3)c2n1] |
| 215 | 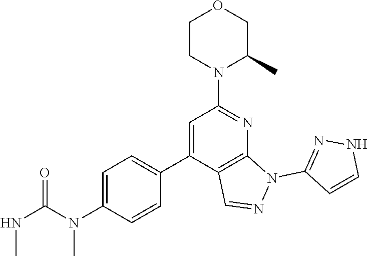 [SMILES: CNC(=O)N(C)c1ccc(-c2cc(N3CCOC[C@H]3C)nc3c2cnn3-c2cc[nH]n2)cc1] | 216 | 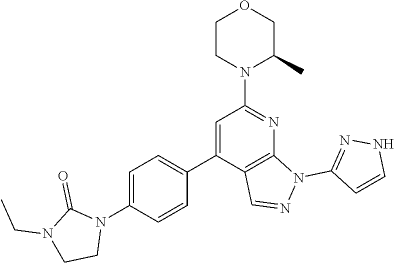 [SMILES: CCN1CCN(c2ccc(-c3cc(N4CCOC[C@H]4C)nc4c3cnn4-c3cc[nH]n3)cc2)C1=O] |
| 217 | 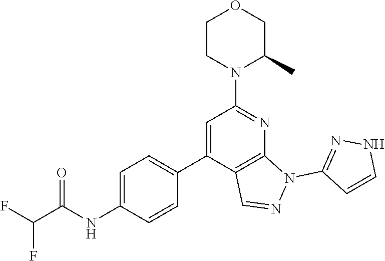 [SMILES: C[C@@H]1COCCN1c1cc(-c2ccc(NC(=O)C(F)F)cc2)c2cnn(-c3cc[nH]n3)c2n1] | 218 | 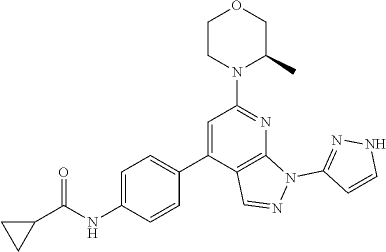 [SMILES: C[C@@H]1COCCN1c1cc(-c2ccc(NC(=O)C3CC3)cc2)c2cnn(-c3cc[nH]n3)c2n1] |
| 219 | 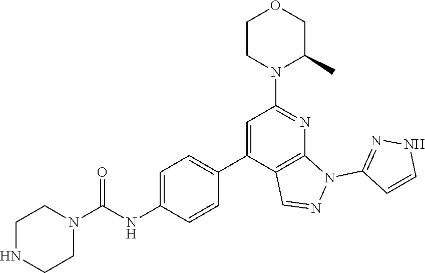 [SMILES: C[C@@H]1COCCN1c1cc(-c2ccc(NC(=O)N3CCNCC3)cc2)c2cnn(-c3cc[nH]n3)c2n1] | 220 | 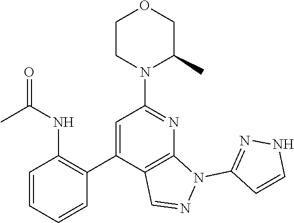 [SMILES: CC(=O)Nc1ccccc1-c1cc(N2CCOC[C@H]2C)nc2c1cnn2-c1cc[nH]n1] |
| 221 | 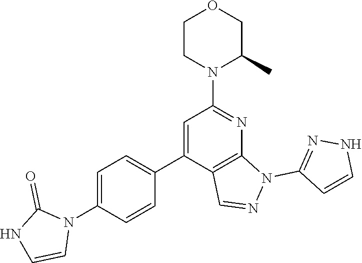 [SMILES: C[C@@H]1COCCN1c1cc(-c2ccc(-n3cc[nH]c3=O)cc2)c2cnn(-c3cc[nH]n3)c2n1] | 222 | 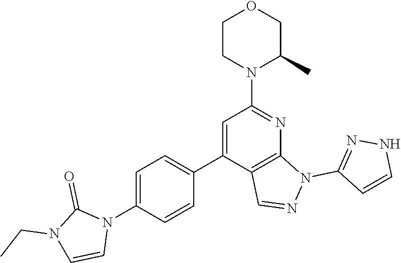 [SMILES: CCn1ccn(-c2ccc(-c3cc(N4CCOC[C@H]4C)nc4c3cnn4-c3cc[nH]n3)cc2)c1=O] |
| 223 | 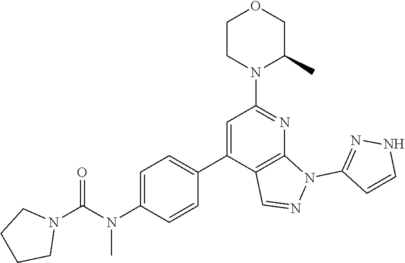 [SMILES: C[C@@H]1COCCN1c1cc(-c2ccc(N(C)C(=O)N3CCCC3)cc2)c2cnn(-c3cc[nH]n3)c2n1] | 224 | 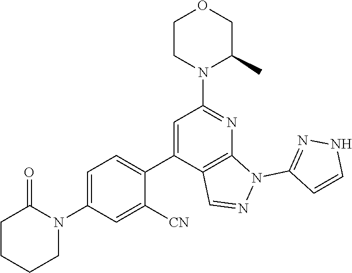 [SMILES: C[C@@H]1COCCN1c1cc(-c2ccc(N3CCCCC3=O)cc2C#N)c2cnn(-c3cc[nH]n3)c2n1] |
| 225 | 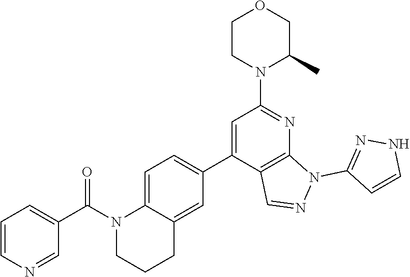 [SMILES: C[C@@H]1COCCN1c1cc(-c2ccc3c(c2)CCCN3C(=O)c2cccnc2)c2cnn(-c3cc[nH]n3)c2n1] | 226 | 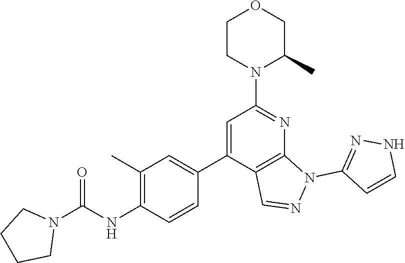 [SMILES: Cc1cc(-c2cc(N3CCOC[C@H]3C)nc3c2cnn3-c2cc[nH]n2)ccc1NC(=O)N1CCCC1] |
| 227 | 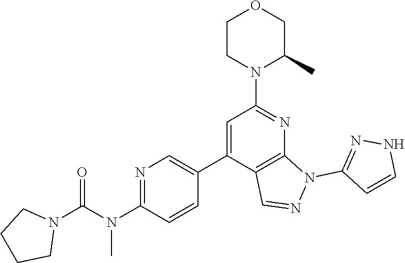 [SMILES: C[C@@H]1COCCN1c1cc(-c2ccc(N(C)C(=O)N3CCCC3)nc2)c2cnn(-c3cc[nH]n3)c2n1] | 228 | 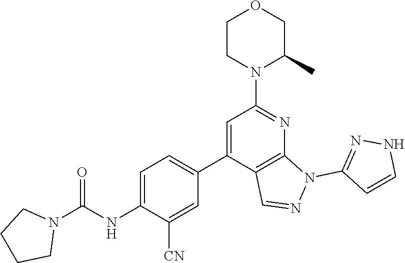 [SMILES: C[C@@H]1COCCN1c1cc(-c2ccc(NC(=O)N3CCCC3)c(C#N)c2)c2cnn(-c3cc[nH]n3)c2n1] |
| 229 | 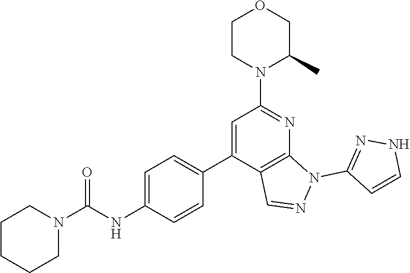 [SMILES: C[C@@H]1COCCN1c1cc(-c2ccc(NC(=O)N3CCCCC3)cc2)c2cnn(-c3cc[nH]n3)c2n1] | 230 | 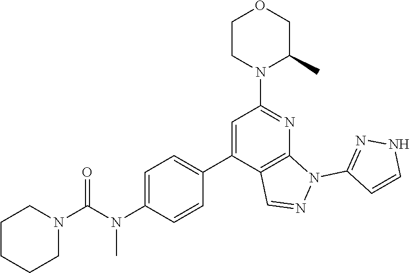 [SMILES: C[C@@H]1COCCN1c1cc(-c2ccc(N(C)C(=O)N3CCCCC3)cc2)c2cnn(-c3cc[nH]n3)c2n1] |
| 231 | 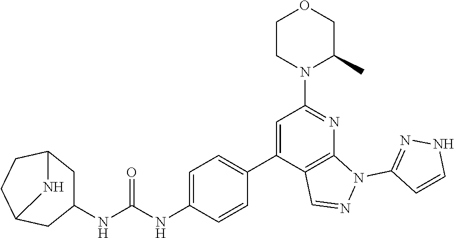 [SMILES: C[C@@H]1COCCN1c1cc(-c2ccc(NC(=O)NC3CC4CCC(C3)N4)cc2)c2cnn(-c3cc[nH]n3)c2n1] | 232 | 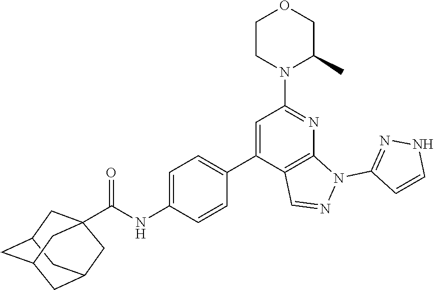 [SMILES: C[C@@H]1COCCN1c1cc(-c2ccc(NC(=O)C34CC5CC(CC(C5)C3)C4)cc2)c2cnn(-c3cc[nH]n3)c2n1] |
| 233 | 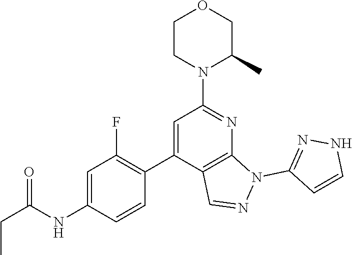 [SMILES: CCC(=O)Nc1ccc(-c2cc(N3CCOC[C@H]3C)nc3c2cnn3-c2cc[nH]n2)c(F)c1] | 234 | 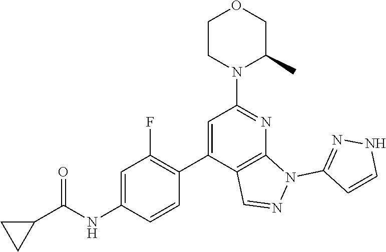 [SMILES: C[C@@H]1COCCN1c1cc(-c2ccc(NC(=O)C3CC3)cc2F)c2cnn(-c3cc[nH]n3)c2n1] |
| 235 | 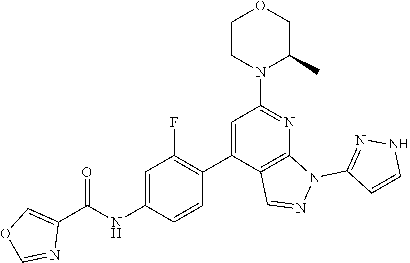 [SMILES: C[C@@H]1COCCN1c1cc(-c2ccc(NC(=O)c3cocn3)cc2F)c2cnn(-c3cc[nH]n3)c2n1] | 236 | 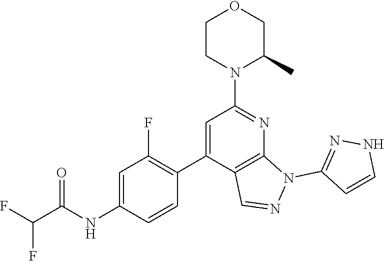 [SMILES: C[C@@H]1COCCN1c1cc(-c2ccc(NC(=O)C(F)F)cc2F)c2cnn(-c3cc[nH]n3)c2n1] |
| 237 | 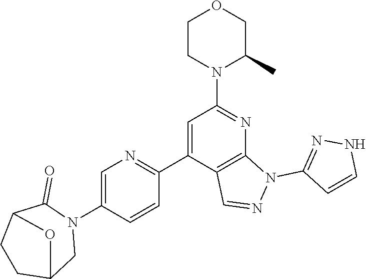 [SMILES: C[C@@H]1COCCN1c1cc(-c2ccc(N3CC4CCC(O4)C3=O)cn2)c2cnn(-c3cc[nH]n3)c2n1] | 238 | 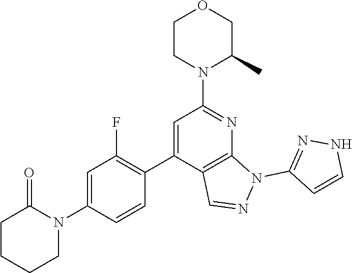 [SMILES: C[C@@H]1COCCN1c1cc(-c2ccc(N3CCCCC3=O)cc2F)c2cnn(-c3cc[nH]n3)c2n1] |
| 239 | 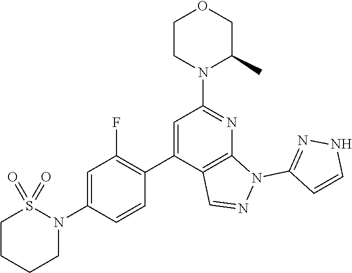 [SMILES: C[C@@H]1COCCN1c1cc(-c2ccc(N3CCCCS3(=O)=O)cc2F)c2cnn(-c3cc[nH]n3)c2n1] | 240 | 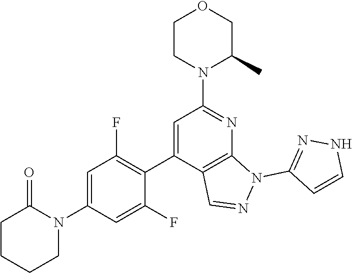 [SMILES: C[C@@H]1COCCN1c1cc(-c2c(F)cc(N3CCCCC3=O)cc2F)c2cnn(-c3cc[nH]n3)c2n1] |
| 241 | 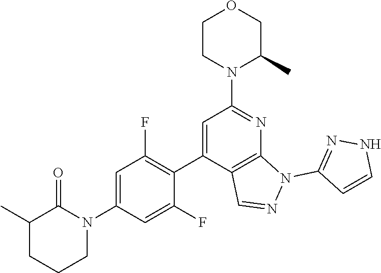 [SMILES: CC1CCCN(c2cc(F)c(-c3cc(N4CCOC[C@H]4C)nc4c3cnn4-c3cc[nH]n3)c(F)c2)C1=O] | 242 | 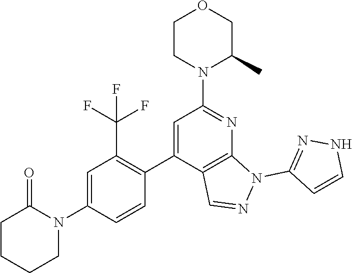 [SMILES: C[C@@H]1COCCN1c1cc(-c2ccc(N3CCCCC3=O)cc2C(F)(F)F)c2cnn(-c3cc[nH]n3)c2n1] |
| 243 | 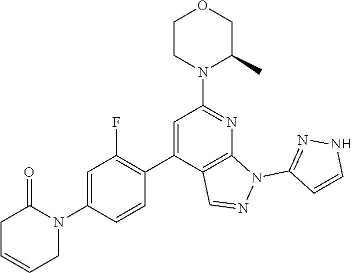 [SMILES: C[C@@H]1COCCN1c1cc(-c2ccc(N3CC=CCC3=O)cc2F)c2cnn(-c3cc[nH]n3)c2n1] | 244 | 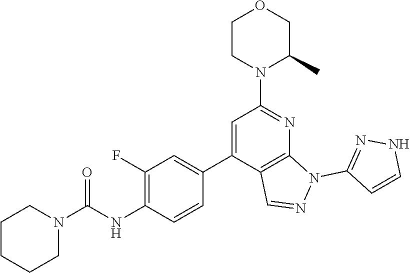 [SMILES: C[C@@H]1COCCN1c1cc(-c2ccc(NC(=O)N3CCCCC3)c(F)c2)c2cnn(-c3cc[nH]n3)c2n1] |
| 245 | 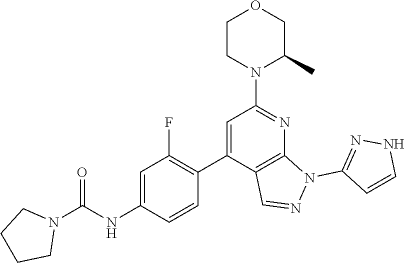 [SMILES: C[C@@H]1COCCN1c1cc(-c2ccc(NC(=O)N3CCCC3)cc2F)c2cnn(-c3cc[nH]n3)c2n1] | 246 | 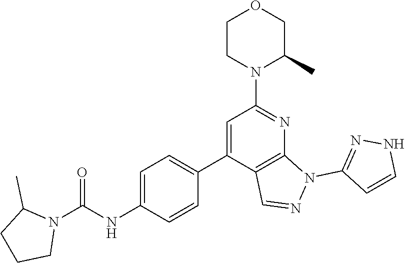 [SMILES: CC1CCCN1C(=O)Nc1ccc(-c2cc(N3CCOC[C@H]3C)nc3c2cnn3-c2cc[nH]n2)cc1] |
| 247 | 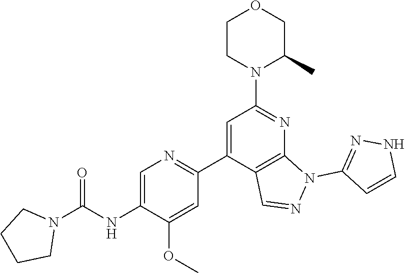 [SMILES: COc1cc(-c2cc(N3CCOC[C@H]3C)nc3c2cnn3-c2cc[nH]n2)ncc1NC(=O)N1CCCC1] | 248 | 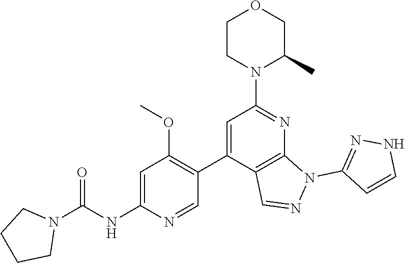 [SMILES: COc1cc(NC(=O)N2CCCC2)ncc1-c1cc(N2CCOC[C@H]2C)nc2c1cnn2-c1cc[nH]n1] |
| 249 | 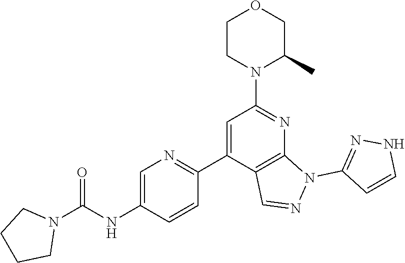 [SMILES: C[C@@H]1COCCN1c1cc(-c2ccc(NC(=O)N3CCCC3)cn2)c2cnn(-c3cc[nH]n3)c2n1] | 250 | 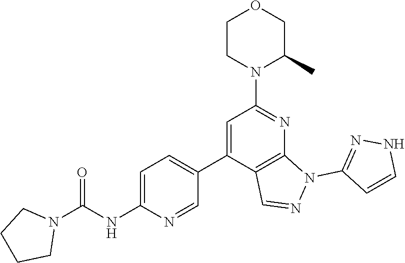 [SMILES: C[C@@H]1COCCN1c1cc(-c2ccc(NC(=O)N3CCCC3)nc2)c2cnn(-c3cc[nH]n3)c2n1] |
| 251 | 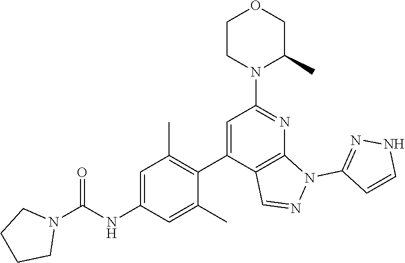 [SMILES: Cc1cc(NC(=O)N2CCCC2)cc(C)c1-c1cc(N2CCOC[C@H]2C)nc2c1cnn2-c1cc[nH]n1] | 252 | 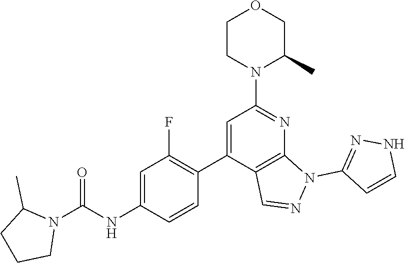 [SMILES: CC1CCCN1C(=O)Nc1ccc(-c2cc(N3CCOC[C@H]3C)nc3c2cnn3-c2cc[nH]n2)c(F)c1] |
| 253 | 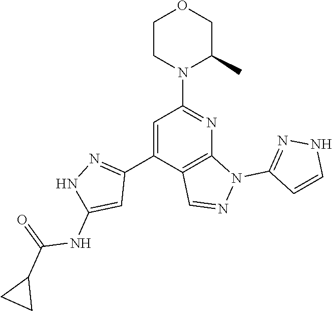 [SMILES: C[C@@H]1COCCN1c1cc(-c2cc(NC(=O)C3CC3)[nH]n2)c2cnn(-c3cc[nH]n3)c2n1] | 254 | 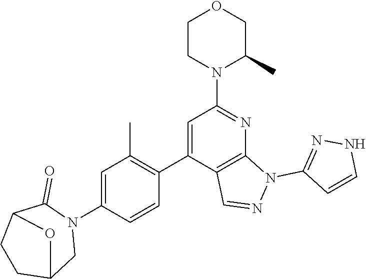 [SMILES: Cc1cc(N2CC3CCC(O3)C2=O)ccc1-c1cc(N2CCOC[C@H]2C)nc2c1cnn2-c1cc[nH]n1] |
| 255 | 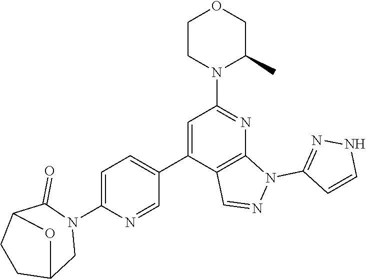 [SMILES: C[C@@H]1COCCN1c1cc(-c2ccc(N3CC4CCC(O4)C3=O)nc2)c2cnn(-c3cc[nH]n3)c2n1] | 256 | 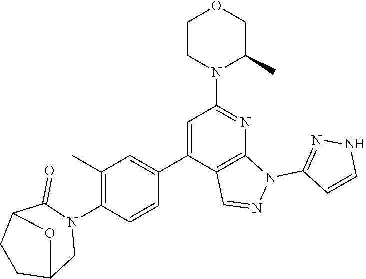 [SMILES: Cc1cc(-c2cc(N3CCOC[C@H]3C)nc3c2cnn3-c2cc[nH]n2)ccc1N1CC2CCC(O2)C1=O] |
| 257 | 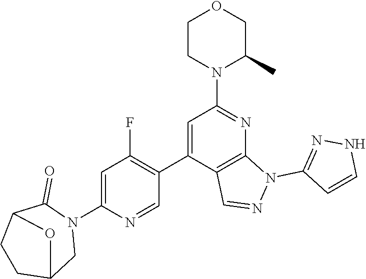 [SMILES: C[C@@H]1COCCN1c1cc(-c2cnc(N3CC4CCC(O4)C3=O)cc2F)c2cnn(-c3cc[nH]n3)c2n1] | 258 | 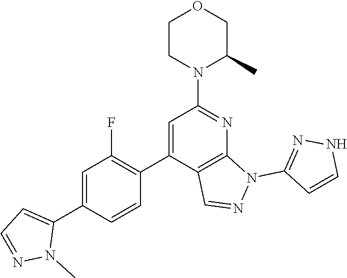 [SMILES: C[C@@H]1COCCN1c1cc(-c2ccc(-c3ccnn3C)cc2F)c2cnn(-c3cc[nH]n3)c2n1] |
| 259 | 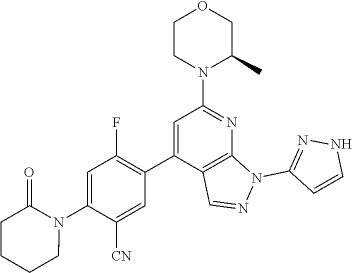 [SMILES: C[C@@H]1COCCN1c1cc(-c2cc(C#N)c(N3CCCCC3=O)cc2F)c2cnn(-c3cc[nH]n3)c2n1] | 260 | 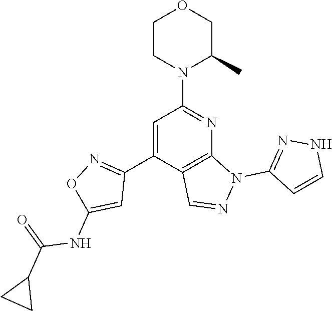 [SMILES: C[C@@H]1COCCN1c1cc(-c2cc(NC(=O)C3CC3)on2)c2cnn(-c3cc[nH]n3)c2n1] |
| 261 | 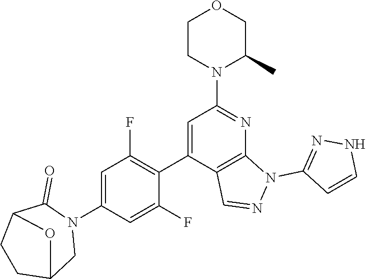 [SMILES: C[C@@H]1COCCN1c1cc(-c2c(F)cc(N3CC4CCC(O4)C3=O)cc2F)c2cnn(-c3cc[nH]n3)c2n1] | 262 | 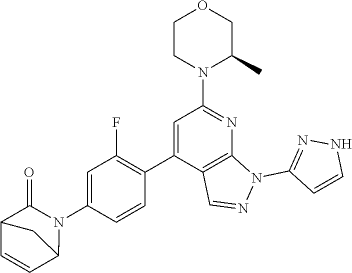 [SMILES: C[C@@H]1COCCN1c1cc(-c2ccc(N3C(=O)C4C=CC3C4)cc2F)c2cnn(-c3cc[nH]n3)c2n1] |
| 263 | 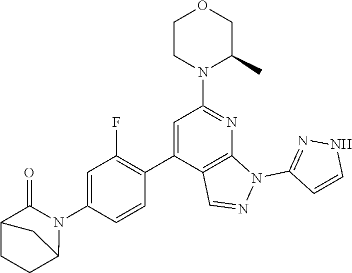 [SMILES: C[C@@H]1COCCN1c1cc(-c2ccc(N3C(=O)C4CCC3C4)cc2F)c2cnn(-c3cc[nH]n3)c2n1] | 264 | 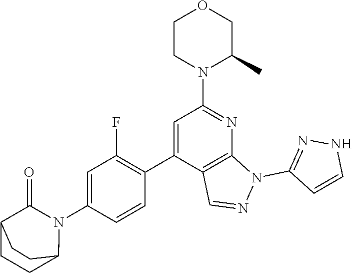 [SMILES: C[C@@H]1COCCN1c1cc(-c2ccc(N3C(=O)C4CCC3CC4)cc2F)c2cnn(-c3cc[nH]n3)c2n1] |
| 265 | 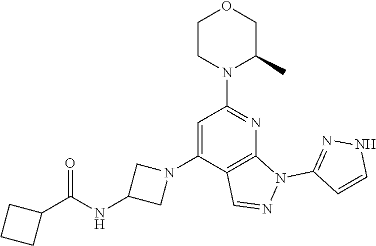 [SMILES: C[C@@H]1COCCN1c1cc(N2CC(NC(=O)C3CCC3)C2)c2cnn(-c3cc[nH]n3)c2n1] | 266 | 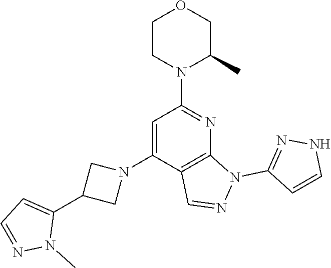 [SMILES: C[C@@H]1COCCN1c1cc(N2CC(c3ccnn3C)C2)c2cnn(-c3cc[nH]n3)c2n1] |
| 267 | 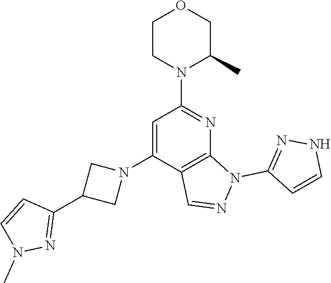 [SMILES: C[C@@H]1COCCN1c1cc(N2CC(c3ccn(C)n3)C2)c2cnn(-c3cc[nH]n3)c2n1] | 268 | 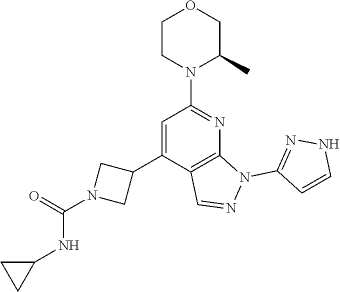 [SMILES: C[C@@H]1COCCN1c1cc(C2CN(C(=O)NC3CC3)C2)c2cnn(-c3cc[nH]n3)c2n1] |
| 269 | 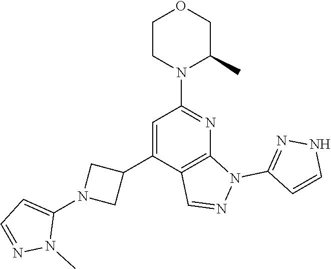 [SMILES: C[C@@H]1COCCN1c1cc(C2CN(c3ccnn3C)C2)c2cnn(-c3cc[nH]n3)c2n1] | 270 | 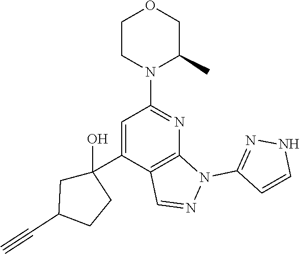 [SMILES: C#CC1CCC(O)(c2cc(N3CCOC[C@H]3C)nc3c2cnn3-c2cc[nH]n2)C1] |
| 271 | 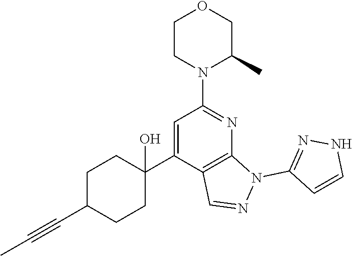 [SMILES: CC#CC1CCC(O)(c2cc(N3CCOC[C@H]3C)nc3c2cnn3-c2cc[nH]n2)CC1] | 272 | 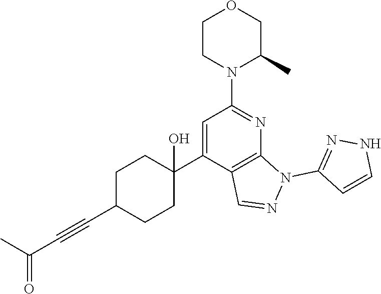 [SMILES: CC(=O)C#CC1CCC(O)(c2cc(N3CCOC[C@H]3C)nc3c2cnn3-c2cc[nH]n2)CC1] |
| 273 | 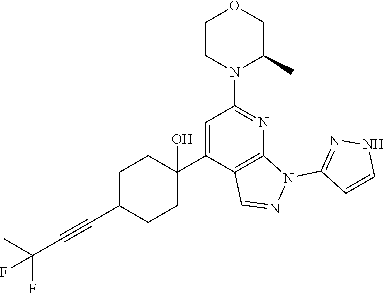 [SMILES: C[C@@H]1COCCN1c1cc(C2(O)CCC(C#CC(C)(F)F)CC2)c2cnn(-c3cc[nH]n3)c2n1] | 274 | 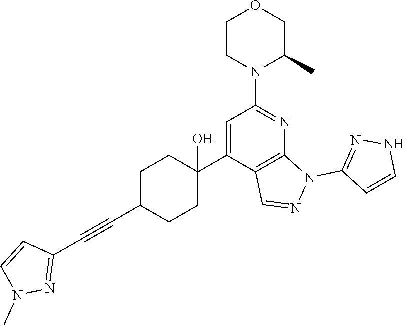 [SMILES: C[C@@H]1COCCN1c1cc(C2(O)CCC(C#Cc3ccn(C)n3)CC2)c2cnn(-c3cc[nH]n3)c2n1] |
| 275 | 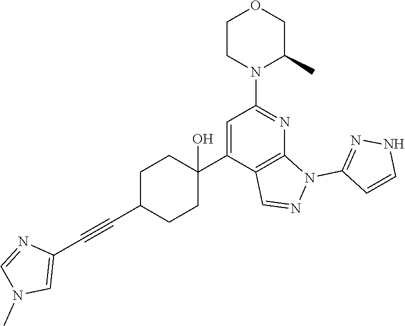 [SMILES: C[C@@H]1COCCN1c1cc(C2(O)CCC(C#Cc3cn(C)cn3)CC2)c2cnn(-c3cc[nH]n3)c2n1] | 276 | 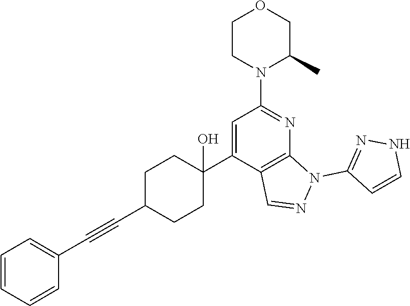 [SMILES: C[C@@H]1COCCN1c1cc(C2(O)CCC(C#Cc3ccccc3)CC2)c2cnn(-c3cc[nH]n3)c2n1] |
| 277 | 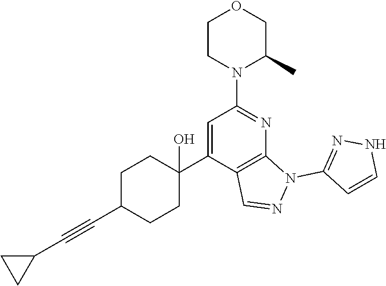 [SMILES: C[C@@H]1COCCN1c1cc(C2(O)CCC(C#CC3CC3)CC2)c2cnn(-c3cc[nH]n3)c2n1] | 278 | 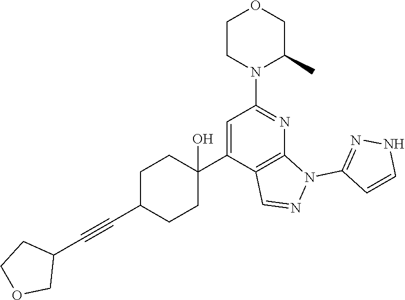 [SMILES: C[C@@H]1COCCN1c1cc(C2(O)CCC(C#CC3CCOC3)CC2)c2cnn(-c3cc[nH]n3)c2n1] |
| 279 | 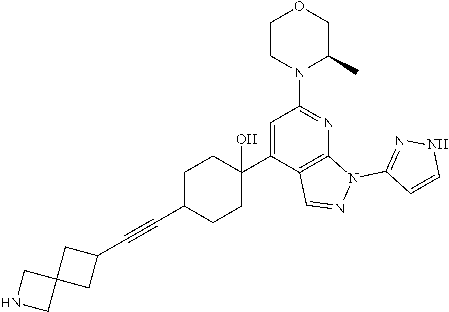 [SMILES: C[C@@H]1COCCN1c1cc(C2(O)CCC(C#CC3CC4(CNC4)C3)CC2)c2cnn(-c3cc[nH]n3)c2n1] | 280 | 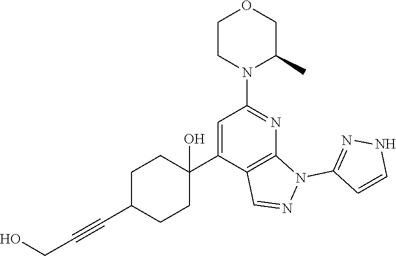 [SMILES: C[C@@H]1COCCN1c1cc(C2(O)CCC(C#CCO)CC2)c2cnn(-c3cc[nH]n3)c2n1] |
| 281 | 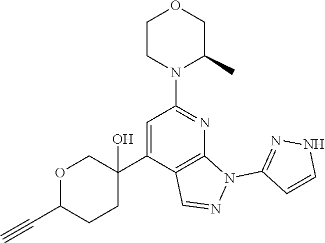 [SMILES: C#CC1CCC(O)(c2cc(N3CCOC[C@H]3C)nc3c2cnn3-c2cc[nH]n2)CO1] | 282 | 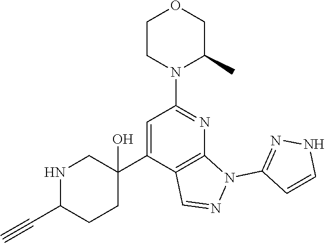 [SMILES: C#CC1CCC(O)(c2cc(N3CCOC[C@H]3C)nc3c2cnn3-c2cc[nH]n2)CN1] |
| 283 | 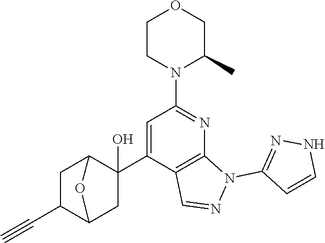 [SMILES: C#CC1CC2OC1CC2(O)c1cc(N2CCOC[C@H]2C)nc2c1cnn2-c1cc[nH]n1] | 284 | 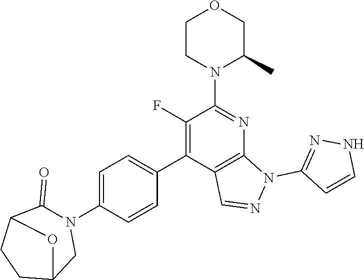 [SMILES: C[C@@H]1COCCN1c1nc2c(cnn2-c2cc[nH]n2)c(-c2ccc(N3CC4CCC(O4)C3=O)cc2)c1F] |
| 285 | 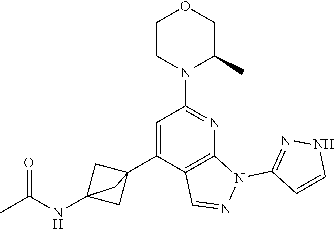 [SMILES: CC(=O)NC12CC(c3cc(N4CCOC[C@H]4C)nc4c3cnn4-c3cc[nH]n3)(C1)C2] | 286 | 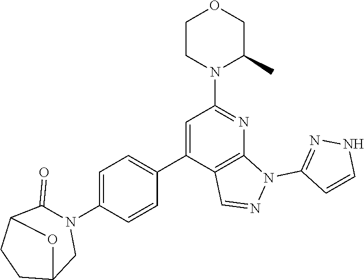 [SMILES: C[C@@H]1COCCN1c1cc(-c2ccc(N3CC4CCC(O4)C3=O)cc2)c2cnn(-c3cc[nH]n3)c2n1] |
| 287 | 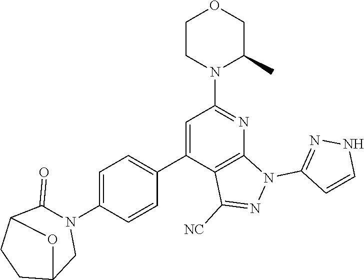 [SMILES: C[C@@H]1COCCN1c1cc(-c2ccc(N3CC4CCC(O4)C3=O)cc2)c2c(C#N)nn(-c3cc[nH]n3)c2n1] | 288 | 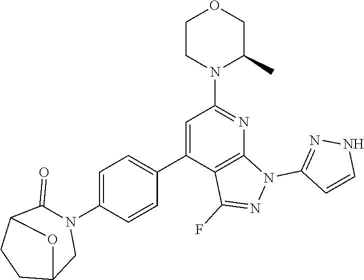 [SMILES: C[C@@H]1COCCN1c1cc(-c2ccc(N3CC4CCC(O4)C3=O)cc2)c2c(F)nn(-c3cc[nH]n3)c2n1] |
| 289 | 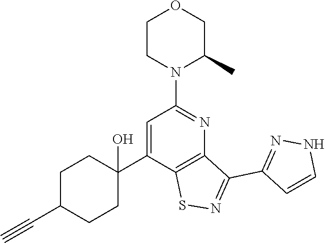 [SMILES: C#CC1CCC(O)(c2cc(N3CCOC[C@H]3C)nc3c(-c4cc[nH]n4)nsc23)CC1] | 290 | 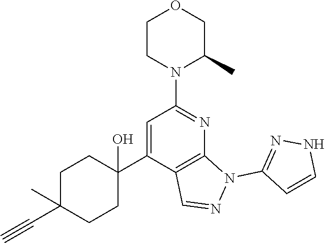 [SMILES: C#CC1(C)CCC(O)(c2cc(N3CCOC[C@H]3C)nc3c2cnn3-c2cc[nH]n2)CC1] |
| 291 | 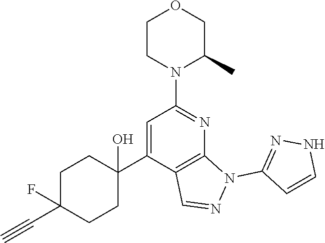 [SMILES: C#CC1(F)CCC(O)(c2cc(N3CCOC[C@H]3C)nc3c2cnn3-c2cc[nH]n2)CC1] | 292 | 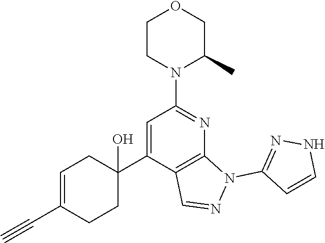 [SMILES: C#CC1=CCC(O)(c2cc(N3CCOC[C@H]3C)nc3c2cnn3-c2cc[nH]n2)CC1] |
| 293 | 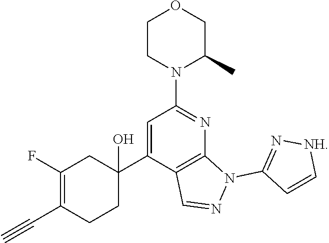 [SMILES: C#CC1=C(F)CC(O)(c2cc(N3CCOC[C@H]3C)nc3c2cnn3-c2cc[nH]n2)CC1] | | |

87. A pharmaceutical composition, comprising the compound, and the stereoisomer, the optical isomer, the pharmaceutical salt, the prodrug and the solvate thereof according to claim 70, and optionally further comprising a pharmaceutically acceptable excipient.

88. A method for treating an ATR-mediated disease, comprising administering to a patient in need thereof a compound, and the stereoisomer, the optical isomer, the pharmaceutical salt, the prodrug and the solvate thereof according to claim 70, or the composition according to claim 87.

89. A method for treating a cancer or tumor-related disease, comprising administering to a patient in need thereof a compound, and the stereoisomer, the optical isomer, the pharmaceutical salt, the prodrug and the solvate thereof according to claim 70, or the composition according to claim 87, wherein the cancer or tumor-related disease is optionally a solid tumor, and optionally, the solid tumor is a digestive tract tumor, wherein the digestive tract tumor is optionally selected from gastric cancer and colorectal cancer.
